(19) United States
(12) Patent Application Publication
(10) Pub. No.: US 2026/0347373 A1
(43) Pub. Date: Nov. 5, 2026

(54) HETEROCYCLIC COMPOUNDS, COMPOSITIONS THEREOF, AND METHODS OF TREATMENT THEREWITH

(71) Applicant: BeOne Medicines I GmbH, Basel (CH)

(72) Inventors: Xirui HU, Beijing (CN); Hanzi SUN, Beijing (CN); Zhikun NI, Beijing (CN); Ce WANG, Beijing (CN); Zhiwei WANG, Beijing (CN)

(73) Assignee: BeOne Medicines I GmbH, Basel (CH)

(21) Appl. No.: 19/682,331

(22) Filed: May 19, 2026

Related U.S. Application Data
(63) Continuation of application No. PCT/CN2024/133892, filed on Nov. 22, 2024.

(30) Foreign Application Priority Data
Nov. 23, 2023 (WO) PCT/CN2023/133671
Jan. 11, 2024 (WO) PCT/CN2024/071755
Feb. 8, 2024 (WO) PCT/CN2024/077032
Apr. 3, 2024 (WO) PCT/CN2024/085955

(51) Int. Cl. C07D 519/00; A61K 31/519; A61K 31/5377; A61K 31/5386; A61K 31/541; A61K 31/553; A61P 35/00; C07B 59/00; C07D 498/22

(52) U.S. Cl. CPC C07D 519/00; A61K 31/519; A61K 31/5377; A61K 31/5386; A61K 31/541; A61K 31/553; A61P 35/00; C07B 59/002; C07D 498/22

(57) ABSTRACT

Provided herein are compounds having the following structure:

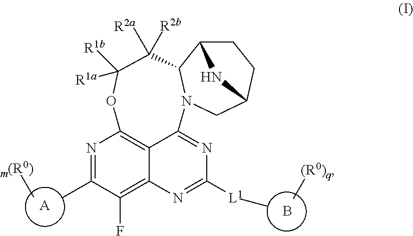

wherein the substituents are as defined herein, compositions comprising a compound, and methods for modulating the activity of KRAS G12D.

### FIELD

Provided herein are heterocyclic compounds that inhibit KRAS activity, as well as their pharmaceutical compositions and methods of use.

### BACKGROUND

Ras is a family of proteins which are associated with cell membrane through their C-terminal membrane targeting region and well known as the molecular switch in intracellular signaling network (Cox A D, Der C J. Ras history: The saga continues. Small GTPases. 2010; 1(1):2-27). Ras proteins bind with either GTP or GDP and switch between “on” and “off” states. By switching to active state, Ras protein can interact with different downstream proteins and activate related signaling pathways (Berndt N, Hamilton A D, Sebti S M. Targeting protein prenylation for cancer therapy. Nat Rev Cancer. 2011; 11(11):775-791). HRas, NRas and KRas are the most well studied proteins in Ras family since these proteins are the most common oncogenes in human cancers (O'Bryan J P. Pharmacological targeting of RAS: Recent success with direct inhibitors. Pharmacol Res. 2019; 139:503-511).

KRas is one of the most frequently mutated genes in human cancers.

Among different cancers, pancreatic cancer is considered as the most KRas-addicted cancer type. KRas mutation is found in 94.1% of pancreatic ductal adenocarcinoma (PDAC). G12D (41%) mutation of KRas is the most predominant mutations in all the KRas mutated PDAC (Waters A M, Der C J. KRAS: The Critical Driver and Therapeutic Target for Pancreatic Cancer. Cold Spring Harb Perspect Med. 2018; 8(9):a031435).

Thus, KRas G12D mutation is a highly attractive target for pancreatic cancer and other cancers with this mutation. As such, small-molecule therapeutic agents that are capable to selectively bind with KRas G12D and inhibit its function would be very useful.

Citation or identification of any reference in this section of this application is not to be construed as an admission that the reference is prior art to the present application.

### SUMMARY

Provided herein are compounds having Formula (I):

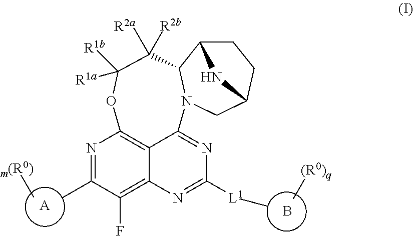

or a pharmaceutically acceptable salt, tautomer, stereoisomer, enantiomer, or atropisomer thereof,
wherein
ring A is unsubstituted or substituted aryl, or unsubstituted or substituted heteroaryl;
moiety B is unsubstituted or substituted cycloalkyl, or unsubstituted or substituted heterocyclyl;
L¹ is a direct bond, or —O—R^{a}—, wherein said R^{a} is, absent or unsubstituted or substituted C₁₋₄ alkylene;
each of R⁰ is, independently, H, halogen, —CN, —OH, unsubstituted or substituted C₁₋₄alkyl, unsubstituted or substituted C₁₋₄alkoxy, unsubstituted or substituted C₁₋₄ alkenyl, unsubstituted or substituted C₃₋₅cycloalkyl, unsubstituted or substituted 3-member to 6-member heterocyclyl, or unsubstituted or substituted amino; or one or more pairs of the R⁰ groups, together with the atom(s) to which they are attached to, form unsubstituted or substituted cycloalkyl, unsubstituted or substituted alkenyl, unsubstituted or substituted heterocyclyl, unsubstituted or substituted aryl, or unsubstituted or substituted heteroaryl;
each of R^{1a}, R^{1b}, R^{2a} and R^{2b}, is, independently, H, halogen, unsubstituted or substituted C₁₋₃alkyl, or R^{1a} and R^{1b}, together form an oxo or a substituted or unsubstituted cyclopropyl;
each of m, and q is, independently, an integer from 0 to the maximum number of the substituent groups allowed on rings A, and B, respectively.

In one embodiment, the compound is selected from Table 2.

In one embodiment, provided herein is a method for inhibiting the activity of KRAS mutant protein in a cell, comprising contacting said cell with a compound provided herein, or a pharmaceutically acceptable salt, tautomer, stereoisomer, enantiomer, or atropisomer thereof, optionally wherein the KRAS mutant protein is KRAS G12D mutant protein.

In one embodiment, provided herein is a method for treatment or prevention of cancer, the method comprising administering to a subject in need thereof a compound provided herein, or a pharmaceutically acceptable salt, tautomer, stereoisomer, enantiomer, or atropisomer thereof, optionally wherein the cancer is mediated by KRAS mutation; preferably KRAS G12D mutation.

### DETAILED DESCRIPTION

### Definitions

As used herein, “KRAS gene” refers to a gene selected from the group consisting of: DIRAS1; DIRAS2; DIRAS3; ERAS; GEM; HRAS; KRAS; MRAS; NKIRAS1; NKIRAS2; NRAS; RALA; RALB; RAP1A; RAP1B; RAP2A; RAP2B; RAP2C; RASD1; RASD2; RASL10A; RASL10B; RASL11A; RASL11B; RASL12; REM1; REM2; RERG; RERGL; RRAD; RRAS; RRAS2, and mutants thereof.

As used herein, “KRAS protein” refers to a protein or an isoform thereof expressed by a KRAS gene (Scolnick E M, Papageoege A G, Shih T Y (1979), “Guanine nucleotide-binding activity for src protein of rat-derived murine sarcoma viruses,” Proc Natl Acad Sci USA. 76 (5): 5355-5559; Kranenburg O (November 2005) “The KRAS oncogene: past, present, and future,” Biochimica et Biophysica Acta (BBA)—Reviews on Cancer, 1756 (2): 81-2).

As used herein, “G12D mutation” refers to the mutation of the 12^{th} amino acid residue located in the G domain of KRAS protein from glycine to aspartic acid.

As used herein, “KRAS G12D” or “G12D” refer to KRAS protein with G12D mutation.

As used herein, and in the specification and the accompanying claims, the indefinite articles “a” and “an” and the definite article “the” include plural as well as single referents, unless the context clearly indicates otherwise.

As used herein, and unless otherwise specified, the terms “about” and “approximately,” when used in connection with doses, amounts, or weight percents of ingredients of a composition or a dosage form, mean a dose, amount, or weight percent that is recognized by one of ordinary skill in the art to provide a pharmacological effect equivalent to that obtained from the specified dose, amount, or weight percent. In certain embodiments, the terms “about” and “approximately,” when used in this context, contemplate a dose, amount, or weight percent within 30%, within 20%, within 15%, within 10%, or within 5%, of the specified dose, amount, or weight percent.

As used herein, and unless otherwise specified, the terms “about” and “approximately,” when used in connection with a numeric value or range of values which is provided to characterize a particular solid form, e.g., a specific temperature or temperature range, such as, for example, that describes a melting, dehydration, desolvation, or glass transition temperature; a mass change, such as, for example, a mass change as a function of temperature or humidity; a solvent or water content, in terms of, for example, mass or a percentage; or a peak position, such as, for example, in analysis by, for example, IR or Raman spectroscopy or XRPD; indicate that the value or range of values may deviate to an extent deemed reasonable to one of ordinary skill in the art while still describing the solid form. Techniques for characterizing crystal forms and amorphous solids include, but are not limited to, thermal gravimetric analysis (TGA), differential scanning calorimetry (DSC), X-ray powder diffractometry (XRPD), single-crystal X-ray diffractometry, vibrational spectroscopy, e.g., infrared (IR) and Raman spectroscopy, solid-state and solution nuclear magnetic resonance (NMR) spectroscopy, optical microscopy, hot stage optical microscopy, scanning electron microscopy (SEM), electron crystallography and quantitative analysis, particle size analysis (PSA), surface area analysis, solubility studies, and dissolution studies. In certain embodiments, the terms “about” and “approximately,” when used in this context, indicate that the numeric value or range of values may vary within 30%, 20%, 15%, 10%, 9%, 8%, 7%, 6%, 5%, 4%, 3%, 2%, 1.5%, 1%, 0.5%, or 0.25% of the recited value or range of values. For example, in some embodiments, the value of an XRPD peak position may vary by up to ±0.2° 2θ (or ±0.2 degree 2θ) while still describing the particular XRPD peak.

As used herein, and unless otherwise specified, the terms “hydrogen” and “H” are interchangeable, and refer to protium, deuterium, or tritium. In one embodiment, the terms “hydrogen” and “H” refer to protium. In one embodiment, the terms “hydrogen” and “H” refer to deuterium. In one embodiment, the terms “hydrogen” and “H” refer to tritium.

An “alkyl” group is a saturated, partially saturated, or unsaturated straight chain or branched non-cyclic hydrocarbon having from 1 to 10 carbon atoms, typically from 1 to 8 carbons or, in some embodiments, from 1 to 6, 1 to 4, or 2 to 6 or carbon atoms. Representative alkyl groups include -methyl, -ethyl, -n-propyl, -n-butyl, -n-pentyl and -n-hexyl; while saturated branched alkyls include -isopropyl, -sec-butyl, -isobutyl, -tert-butyl, -isopentyl, -neopentyl, tert-pentyl, -2-methylpentyl, -3-methylpentyl, -4-methylpentyl, -2,3-dimethylbutyl and the like. Examples of unsaturated alkyl groups include, but are not limited to, vinyl, allyl, —CH═CH(CH₃), —CH═C(CH₃)₂, —C(CH₃)═CH₂, —C(CH₃)═CH(CH₃), —C(CH₂CH₃)═CH₂, —C≡CH, —C≡C(CH₃), —C≡C(CH₂CH₃), —CH₂C≡CH, —CH₂C≡C(CH₃) and —CH₂C≡C(CH₇CH₃), among others. An alkyl group can be substituted or unsubstituted. When the alkyl groups described herein are said to be “substituted,” they may be substituted with any substituent or substituents as those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonato; phosphine; thiocarbonyl; sulfonyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; B(OH)₂, or O(alkyl)aminocarbonyl.

An “alkylene” group is a saturated, partially saturated, or unsaturated straight divalent group having from 1 to 10 carbon atoms, typically from 1 to 8 carbons or, in some embodiments, from 1 to 6, 1 to 4, or 2 to 6 or carbon atoms. Representative alkylene groups include —CH₂—, —CH₂CH₂—, —CH(CH₃)—, —C(CH₃)₂—, CH₂CH₂CH₂—, CH(CH₃)CH₂—, —C═CH₂, or —C═CH₂—CH₂—.

An “alkenyl” group is a straight chain or branched non-cyclic hydrocarbon having from 2 to 10 carbon atoms, typically from 2 to 8 carbon atoms, and including at least one carbon-carbon double bond. Representative straight chain and branched (C₂-C₈)alkenyls include -vinyl, -allyl, -1-butenyl, -2-butenyl, -isobutylenyl, -1-pentenyl, 2pentenyl, -3-methyl-1-butenyl, -2-methyl-2-butenyl, -2,3-dimethyl-2-butenyl, -1-hexenyl, 2-hexenyl, -3-hexenyl, -1-heptenyl, -2-heptenyl, -3-heptenyl, -1-octenyl, -2-octenyl, 3octenyl and the like. The double bond of an alkenyl group can be unconjugated or conjugated to another unsaturated group. An alkenyl group can be unsubstituted or substituted.

An “alkynyl” group refers to a monovalent hydrocarbon moiety containing at least two carbon atoms and one or more carbon-carbon triple bonds. Alkynyl is optionally substituted and can be linear, branched, or cyclic. Alkynyl includes, but is not limited to, those moieties having 2-20 carbon atoms, i.e., C₂₋₂₀ alkynyl; 2-12 carbon atoms, i.e., C₂₋₁₂ alkynyl; 2-8 carbon atoms, i.e., C₂₋₈ alkynyl; 2-6 carbon atoms, i.e., C₂₋₆ alkynyl; and 2-4 carbon atoms, i.e., C₂₋₄ alkynyl. Examples of alkynyl moieties include, but are not limited to ethynyl, propynyl, and butynyl.

A “cycloalkyl” group is a saturated, partially saturated, or unsaturated cyclic alkyl group of from 3 to 10 carbon atoms having a single cyclic ring or multiple condensed or bridged rings which can be optionally substituted with from 1 to 3 alkyl groups. In some embodiments, the cycloalkyl group has 3 to 8 ring members, whereas in other embodiments the number of ring carbon atoms ranges from 3 to 5, 3 to 6, or 3 to 7. A cycloalkyl comprising more than one ring may be fused, spiro, or bridged, or combinations thereof. Such cycloalkyl groups include, by way of example, single ring structures such as cyclopropyl, cyclobutyl, cyclopentyl, cyclohexyl, cycloheptyl, cyclooctyl, 1-methylcyclopropyl, 2-methylcyclopentyl, 2-methylcyclooctyl, and the like, or multiple or bridged ring structures such as 1-bicyclo[1.1.1]pentyl, bicyclo[2.1.1]hexyl, bicyclo[2.2.1]heptyl, bicyclo[2.2.2]octyl, adamantyl and the like. Examples of unsaturated cycloalkyl groups include cyclohexenyl, cyclopentenyl, cyclohexadienyl, butadienyl, pentadienyl, hexadienyl, among others. A cycloalkyl group can be substituted or unsubstituted. Such substituted cycloalkyl groups include, by way of example, cyclohexanol and the like.

A “bridged” bicyclic ring system includes two rings sharing three, four, or five adjacent ring atoms. As used herein, the term “bridge” refers to an atom or chain of atoms that connects two different parts of a molecule. Two atoms connected through a bridge (usually but not always two tertiary carbon atoms) are called “bridgeheads”. In addition to the bridge, the two bridgeheads are connected by at least two individual atoms or atomic chains. Examples of bridged bicyclic ring systems include adamantanyl, norbornanyl, bicyclo [3.2.1] octyl, bicyclo [2.2.2] octyl, bicyclo [3.3.1] nonyl, bicyclo [3.2.3] nonyl, 2-oxa-bicyclo [2.2.2] octyl, 1-aza-bicyclo [2.2.2] octyl, 3-aza-bicyclo [3.2.1] octyl, and 2, Examples include, but are not limited to, 6-dioxa-tricyclo [3.3.1.03,7] nonyl. In one embodiment, the bridge is unsubstituted or substituted —(CH₂)ₙ—, wherein n is 1, 2, 3, 4, or 5. In one embodiment, the bridge is —CH₂—. In one embodiment, the bridge is —(CH₂)₂—. In one embodiment, the bridge is —(CH₂)₃—. In one embodiment, the bridge is —CH₂—O—CH₂—. A “spiro” bicyclic ring system shares a single ring atom (usually a quaternary carbon atom) between two rings.

A “fusion atom” is an atom that is shared by two or more rings in a fused ring system.

An “aryl” group is a fully aromatic carbocyclic group of from 6 to 14 carbon atoms having a single ring (e.g., phenyl) or multiple condensed rings (e.g., naphthyl or anthryl). In some embodiments, aryl groups contain 6-14 carbons, and in others from 6 to 12 or even 6 to 10 carbon atoms in the ring portions of the groups. Particular aryls include phenyl, biphenyl, naphthyl and the like. An aryl group can be substituted or unsubstituted.

A “heterocyclyl” is an aromatic (also referred to as heteroaryl) or non-aromatic cycloalkyl in which one to four of the ring carbon atoms are independently replaced with a heteroatom from the group consisting of O, S and N. In some embodiments, heterocyclyl groups include 3 to 10 ring members, whereas other such groups have 3 to 5, 3 to 6, or 3 to 8 ring members. Heterocyclyls can also be bonded to other groups at any ring atom (i.e., at any carbon atom or heteroatom of the heterocyclic ring). A heterocyclyl group can be substituted or unsubstituted. A heterocyclyl group may include multiple condensed rings including, but are not limited to, bicyclic, tricyclic, and quadracyclic rings, as well as bridged or spirocyclic ring systems. Heterocyclyl groups encompass unsaturated, partially saturated and saturated ring systems, such as, for example, imidazolyl, imidazolinyl and imidazolidinyl (e.g., imidazolidin-4-one or imidazolidin-2,4-dionyl) groups. The phrase heterocyclyl includes fused ring species, including those comprising fused aromatic and non-aromatic groups, such as, for example, 1- and 2-aminotetraline, benzotriazolyl (e.g., 1H-benzo[d][1,2,3]triazolyl), benzimidazolyl (e.g., 1H-benzo[d]imidazolyl), 2,3-dihydrobenzo[1,4]dioxinyl, and benzo[1,3]dioxolyl. The phrase also includes bridged polycyclic ring systems containing a heteroatom such as, but not limited to, quinuclidyl. Representative examples of a heterocyclyl group include, but are not limited to, aziridinyl, azetidinyl, azepanyl, oxetanyl, pyrrolidyl, imidazolidinyl (e.g., imidazolidin-4-onyl or imidazolidin-2,4-dionyl), pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, benzisoxazolyl (e.g., benzo[d]isoxazolyl), thiazolyl, thiazolinyl, isothiazolyl, thiadiazolyl, oxadiazolyl, piperidyl, piperazinyl (e.g., piperazin-2-onyl), morpholinyl, thiomorpholinyl, tetrahydropyranyl (e.g., tetrahydro-2H-pyranyl), tetrahydrothiopyranyl, oxathianyl, dioxyl, dithianyl, pyranyl, pyridyl, pyrimidyl, pyridazinyl, pyrazinyl, triazinyl, dihydropyridyl, dihydrodithiinyl, dihydrodithionyl, 1,4-dioxaspiro[4.5]decanyl, 2-oxo-1-oxa-3,8-diazaspiro[4.5]decane, 1-oxo-2,8-diazaspiro[4.5]decane, 3-oxo-2,8-diazaspiro[4.5]decane, 3-oxo-1-oxa-4,9-diazaspiro[5.5]undecane, 2-oxo-1-oxa-3,9-diazaspiro[5.5]undecane, homopiperazinyl, quinuclidyl, indolyl (e.g., indolyl-2-onyl or isoindolin-1-onyl), indolinyl, isoindolyl, isoindolinyl, azaindolyl (pyrrolopyridyl or 1H-pyrrolo[2,3-b]pyridyl), indazolyl, indolizinyl, benzotriazolyl (e.g., 1H-benzo[d][1,2,3]triazolyl), benzimidazolyl (e.g., 1H-benzo[d]imidazolyl or 1H-benzo[d]imidazol-2(3H)-onyl), benzofuranyl, benzothiophenyl, benzothiazolyl, benzoxadiazolyl, benzoxazinyl, benzodithiinyl, benzoxathiinyl, benzothiazinyl, benzoxazolyl (i.e., benzo[d]oxazolyl), benzothiazolyl, benzothiadiazolyl, benzo[1,3]dioxolyl, pyrazolopyridyl (for example, 1H-pyrazolo[3,4-b]pyridyl, 1H-pyrazolo[4,3-b]pyridyl), imidazopyridyl (e.g., azabenzimidazolyl or 1H-imidazo[4,5-b]pyridyl), triazolopyridyl, isoxazolopyridyl, purinyl, xanthinyl, adeninyl, guaninyl, quinolinyl, isoquinolinyl (e.g., 3,4-dihydroisoquinolin-1(2H)-onyl), quinolizinyl, quinoxalinyl, quinazolinyl, cinnolinyl, phthalazinyl, naphthyridinyl, pteridinyl, thianaphthalenyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, tetrahydropyrimidin-2(1H)-one and tetrahydroquinolinyl groups. Representative non-aromatic heterocyclyl groups do not include fused ring species that comprise a fused aromatic group. Examples of non-aromatic heterocyclyl groups include aziridinyl, azetidinyl, azepanyl, pyrrolidyl, imidazolidinyl (e.g., imidazolidin-4-onyl or imidazolidin-2,4-dionyl), pyrazolidinyl, thiazolidinyl, tetrahydrothiophenyl, tetrahydrofuranyl, piperidyl, piperazinyl (e.g., piperazin-2-onyl), morpholinyl, thiomorpholinyl, tetrahydropyranyl (e.g., tetrahydro-2H-pyranyl), tetrahydrothiopyranyl, oxathianyl, dithianyl, 1,4-dioxaspiro[4.5]decanyl, homopiperazinyl, quinuclidyl, or tetrahydropyrimidin-2(1H)-one. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed below.

A “heteroaryl” group is an aryl ring system having one to four heteroatoms as ring atoms in a heteroaromatic ring system, wherein the remainder of the atoms are carbon atoms. In some embodiments, heteroaryl groups contain 3 to 6 ring atoms, and in others from 6 to 9 or even 6 to 10 atoms in the ring portions of the groups. Suitable heteroatoms include oxygen, sulfur and nitrogen. In certain embodiments, the heteroaryl ring system is monocyclic or bicyclic. Non-limiting examples include but are not limited to, groups such as pyrrolyl, pyrazolyl, imidazolyl, triazolyl, tetrazolyl, oxazolyl, isoxazolyl, benzisoxazolyl (e.g., benzo[d]isoxazolyl), thiazolyl, pyrolyl, pyridazinyl, pyrimidyl, pyrazinyl, thiophenyl, benzothiophenyl, furanyl, benzofuranyl, indolyl (e.g., indolyl-2-onyl or isoindolin-1-onyl), azaindolyl (pyrrolopyridyl or 1H-pyrrolo[2,3-b]pyridyl), indazolyl, benzimidazolyl (e.g., 1H-benzo[d]imidazolyl), imidazopyridyl (e.g., azabenzimidazolyl or 1H-imidazo[4,5-b]pyridyl), pyrazolopyridyl, triazolopyridyl, benzotriazolyl (e.g., 1H-benzo[d][1,2,3]triazolyl), benzoxazolyl (e.g., benzo[d]oxazolyl), benzothiazolyl, benzothiadiazolyl, isoxazolopyridyl, thianaphthalenyl, purinyl, xanthinyl, adeninyl, guaninyl, quinolinyl, isoquinolinyl (e.g., 3,4-dihydroisoquinolin-1(2H)-onyl), tetrahydroquinolinyl, quinoxalinyl, and quinazolinyl groups.

As used herein, “spirocyclic ring” refers to two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings.

A “cycloalkylalkyl” group is a radical of the formula: -alkyl-cycloalkyl, wherein alkyl and cycloalkyl are as defined above. Substituted cycloalkylalkyl groups may be substituted at the alkyl, the cycloalkyl, or both the alkyl and the cycloalkyl portions of the group. Representative cycloalkylalkyl groups include but are not limited to methylcyclopropyl, methylcyclobutyl, methylcyclopentyl, methylcyclohexyl, ethylcyclopropyl, ethylcyclobutyl, ethylcyclopentyl, ethylcyclohexyl, propylcyclopentyl, propylcyclohexyl and the like.

An “aralkyl” group is a radical of the formula: -alkyl-aryl, wherein alkyl and aryl are defined above. Substituted aralkyl groups may be substituted at the alkyl, the aryl, or both the alkyl and the aryl portions of the group. Representative aralkyl groups include but are not limited to benzyl and phenethyl groups and fused (cycloalkylaryl)alkyl groups such as 4-ethyl-indanyl.

An “heterocyclylalkyl” group is a radical of the formula: -alkyl-heterocyclyl, wherein alkyl and heterocyclyl are defined above. Substituted heterocyclylalkyl groups may be substituted at the alkyl, the heterocyclyl, or both the alkyl and the heterocyclyl portions of the group. Representative heterocylylalkyl groups include but are not limited to 4-ethyl-morpholinyl, 4-propylmorpholinyl, furan-2-yl methyl, furan-3-yl methyl, pyridin-3-yl methyl, tetrahydrofuran-2-yl ethyl, and indol-2-yl propyl.

A “halogen” is fluorine, chlorine, bromine or iodine.

A “hydroxyalkyl” group is an alkyl group as described above substituted with one or more hydroxy groups.

An “alkoxy” or “alkoxyl” group is —O-(alkyl), wherein alkyl is defined above.

An “alkoxyalkyl” group is -(alkyl)-O-(alkyl), wherein alkyl is defined above.

An “amino” group is a radical of the formula: —NH₂.

An “alkylamino” group is a radical of the formula: —NH-alkyl or —N(alkyl)₂, wherein each alkyl is independently as defined above.

A “carboxy” group is a radical of the formula: —C(O)OH.

An “aminocarbonyl” group is a radical of the formula: —C(O)N(R^{#})₂, —C(O)NH(R^{#}) or —C(O)NH₂, wherein each R^{#} is independently a substituted or unsubstituted alkyl, cycloalkyl, aryl, aralkyl, heterocyclyl or heterocyclyl group as defined herein.

An “acylamino” group is a radical of the formula: —NHC(O)(R^{#}) or —N(alkyl)C(O)(R^{#}), wherein each alkyl and R^{#} are independently as defined above.

A “sulfonylamino” group is a radical of the formula: —NHSO₂(R^{#}) or —N(alkyl)SO₂(R^{#}), wherein each alkyl and R^{#} are defined above.

A “urea” group is a radical of the formula: —N(alkyl)C(O)N(R^{#})₂, —N(alkyl)C(O)NH(R^{#}), —N(alkyl)C(O)NH₂, —NHC(O)N(R^{#})₂, —NHC(O)NH(R^{#}), or —NH(CO)NHR^{#}, wherein each alkyl and R^{#} are independently as defined above.

When the groups described herein, with the exception of alkyl group, are said to be “substituted,” they may be substituted with any appropriate substituent or substituents. Illustrative examples of substituents are those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonato; phosphine; thiocarbonyl; sulfonyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; oxygen (═O); B(OH)₂, O(alkyl)aminocarbonyl; cycloalkyl, which may be monocyclic or fused or non-fused polycyclic (e.g., cyclopropyl, cyclobutyl, cyclopentyl, or cyclohexyl), or a heterocyclyl, which may be monocyclic or fused or non-fused polycyclic (e.g., pyrrolidyl, piperidyl, piperazinyl, morpholinyl, or thiazinyl); monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridyl, quinolinyl, isoquinolinyl, acridinyl, pyrazinyl, pyridazinyl, pyrimidyl, benzimidazolyl, benzothiophenyl, or benzofuranyl) aryloxy; aralkyloxy; heterocyclyloxy; and heterocyclyl alkoxy.

As used herein, the term “substituent” refers to an atom or a group of atoms replacing a hydrogen atom on the parent molecule. The term “optionally substituted” means that the specified group is unsubstituted or substituted by one or more substituents, independently chosen from the group of possible substituents. When indicating the number of substituents, the term “one or more” refers to a range from one substituent to the highest possible number of substituents, i.e. replacement of from one hydrogen up to replacement of all hydrogen atoms by substituents, for example, 1 to 8, 1 to 7, 1 to 6, 1 to 5, 1 to 4, 1 to 3, 1 or 2, or one substituents. And it will be understood by those skilled in the art with respect to any chemical group containing one or more substituents that such groups are not intended to introduce any substitution or substitution patterns that are sterically impractical and/or physically non-feasible.

As used herein, the term “pharmaceutically acceptable salt(s)” refers to a salt prepared from a pharmaceutically acceptable non-toxic acid or base including an inorganic acid and base and an organic acid and base. Suitable pharmaceutically acceptable base addition salts of the compounds of formula (I) include, but are not limited to those well-known in the art, see for example, Remington's Pharmaceutical Sciences, 18^{th} eds., Mack Publishing, Easton PA (1990) or Remington: The Science and Practice of Pharmacy, 19^{th} eds., Mack Publishing, Easton PA (1995).

As used herein and unless otherwise indicated, the term “stereoisomer” or “stereomerically pure” means one stereoisomer of a compound that is substantially free of other stereoisomers of that compound. For example, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The compounds can have chiral centers and can occur as racemates, individual enantiomers or diastereomers, and mixtures thereof. All such isomeric forms are included within the embodiments disclosed herein, including mixtures thereof.

The use of stereomerically pure forms of such compounds, as well as the use of mixtures of those forms, are encompassed by the embodiments disclosed herein. For example, mixtures comprising equal or unequal amounts of the enantiomers of a particular compound may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN, 1972).

It should also be noted the compounds can include E and Z isomers, or a mixture thereof, and cis and trans isomers or a mixture thereof. In certain embodiments, the compounds are isolated as either the E or Z isomer. In other embodiments, the compounds are a mixture of the E and Z isomers.

As used herein and unless otherwise indicated, “atropisomers” refer to stereoisomers resulting from hindered rotation about a single bond axis where the rotational barrier is high enough to allow for the isolation of the individual rotational isomers.

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in aqueous solution, pyrazoles may exhibit the following isomeric forms, which are referred to as tautomers of each other:

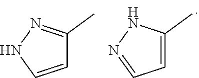

As readily understood by one skilled in the art, a wide variety of functional groups and other structures may exhibit tautomerism and all tautomers of compounds of formula (I) are within the scope of the present invention.

“Treating” as used herein, means an alleviation, in whole or in part, of a disorder, disease or condition, or one or more of the symptoms associated with a disorder, disease, or condition, or slowing or halting of further progression or worsening of those symptoms, or alleviating or eradicating the cause(s) of the disorder, disease, or condition itself. In some embodiments, “treating” means an alleviation, in whole or in part, of a disorder, disease or condition, or a slowing, or halting of further progression or worsening of those symptoms. In another embodiment, “treating” means and alleviation, in whole or in part, of a disorder, disease or condition, or symptoms associated with a condition, wherein the condition is treatable or preventable by inhibition of KRAS; preferably KRAS G12D.

“Preventing” as used herein, means a method of delaying and/or precluding the onset, recurrence or spread, in whole or in part, of a disorder, disease or condition; barring a subject from acquiring a disorder, disease, or condition; or reducing a subject's risk of acquiring a disorder, disease, or condition. In one embodiment, the condition is a condition, treatable or preventable by inhibition of KRAS; preferably KRAS G12D.

The term “effective amount” in connection with a compound means an amount capable of treating or preventing a disorder, disease or condition, or symptoms thereof, disclosed herein.

The term “subject” means a mammal, in another embodiment a human.

### Compounds

Provided herein are compounds having the following formula (Ia):

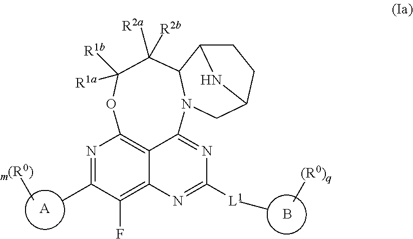

and a pharmaceutically acceptable salt, tautomer, stereoisomer, enantiomer, or atropisomer thereof,
wherein
ring A is unsubstituted or substituted aryl, or unsubstituted or substituted heteroaryl;
moiety B is unsubstituted or substituted cycloalkyl, or unsubstituted or substituted heterocyclyl;
L¹ is a direct bond, or —O—R^{a}—, wherein said R^{a} is, absent or unsubstituted or substituted C₁₋₄alkylene;
each of R⁰ is, independently, H, halogen, —CN, —OH, unsubstituted or substituted C₁₋₄alkyl, unsubstituted or substituted C₁₋₄alkoxy, unsubstituted or substituted C₁₋₄ alkenyl, unsubstituted or substituted C₃₋₅cycloalkyl, unsubstituted or substituted 3-member to 5-member heterocyclyl, or unsubstituted or substituted amino; or one or more pairs of the R⁰ groups, together with the atom(s) to which they are attached to, form unsubstituted or substituted cycloalkyl, unsubstituted or substituted alkenyl, unsubstituted or substituted heterocyclyl, unsubstituted or substituted aryl, or unsubstituted or substituted heteroaryl;
each of R^{1a}, R^{1b}, R^{2a} and R^{2b}, is, independently, H, halogen, unsubstituted or substituted C₁₋₃alkyl, or R^{1a} and R^{1b}, together form an oxo or a substituted or unsubstituted cyclopropyl;
each of m, and q is, independently, an integer from 0 to the maximum number of the substituent groups allowed on rings A, and B, respectively.

Provided herein are compounds having the following formula (I):

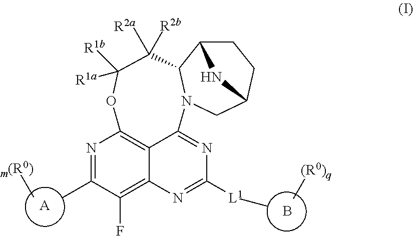

and a pharmaceutically acceptable salt, tautomer, stereoisomer, enantiomer, or atropisomer thereof,
wherein
ring A is unsubstituted or substituted aryl, or unsubstituted or substituted heteroaryl;
moiety B is unsubstituted or substituted cycloalkyl, unsubstituted or substituted heterocyclyl;
L¹ is a direct bond, or —O—R^{a}—, wherein said R^{a} is, absent or unsubstituted or substituted C₁₋₄ alkylene;
each of R⁰ is, independently, H, halogen, —CN, —OH, unsubstituted or substituted C₁₋₄alkyl, unsubstituted or substituted C₁₋₄alkoxy, unsubstituted or substituted C₁₋₄ alkenyl, unsubstituted or substituted C₃₋₅cycloalkyl, unsubstituted or substituted 3-member to 6-member heterocyclyl, or unsubstituted or substituted amino; or one or more pairs of the R⁰ groups, together with the atom(s) to which they are attached to, form unsubstituted or substituted cycloalkyl, unsubstituted or substituted alkenyl, unsubstituted or substituted heterocyclyl, unsubstituted or substituted aryl, or unsubstituted or substituted heteroaryl;
each of R^{1a}, R^{1b}, R^{2a} and R^{2b}, is, independently, H, halogen, unsubstituted or substituted C₁₋₃alkyl, or R^{1a} and R^{1b}, together form an oxo or a substituted or unsubstituted cyclopropyl;
each of m, and q is, independently, an integer from 0 to the maximum number of the substituent groups allowed on rings A, and B, respectively.

In some embodiments, said compound is not the compound selected from Table 1 as described in PCT application PCT/CN2023/112174.

TABLE 1

| Compound No. | Structure |
|---|---|
| 1 | 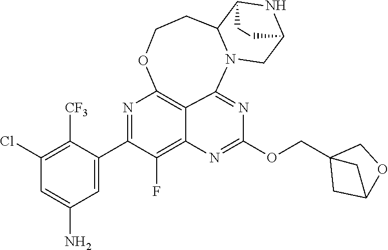 [SMILES: Nc1cc(Cl)c(C(F)(F)F)c(-c2nc3c4c(nc(OCC56COC(C5)C6)nc4c2F)N2C[C@H]4CC[C@H](N4)C2CCO3)c1] |
| 2 | 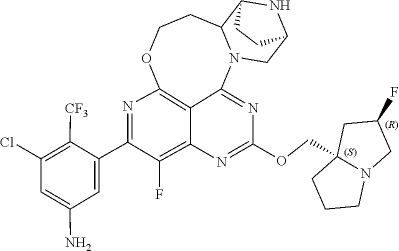 |
| 3 | 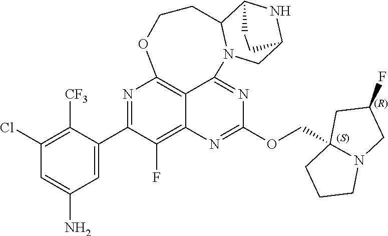 |
| 4 | 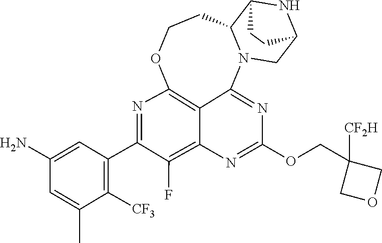 [SMILES: Cc1cc(N)cc(-c2nc3c4c(nc(OCC5(C(F)F)COC5)nc4c2F)N2C[C@H]4CC[C@H](N4)[C@H]2CCO3)c1C(F)(F)F] |
| 5 | 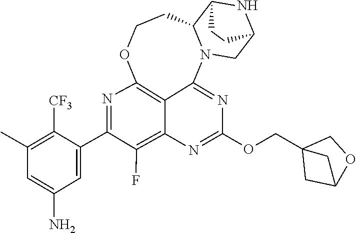 [SMILES: Cc1cc(N)cc(-c2nc3c4c(nc(OCC56COC(C5)C6)nc4c2F)N2C[C@H]4CC[C@H](N4)[C@H]2CCO3)c1C(F)(F)F] |
| 6 | 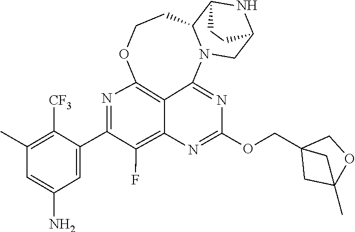 [SMILES: Cc1cc(N)cc(-c2nc3c4c(nc(OCC56COC(C)(C5)C6)nc4c2F)N2C[C@H]4CC[C@H](N4)[C@H]2CCO3)c1C(F)(F)F] |
| 7 | 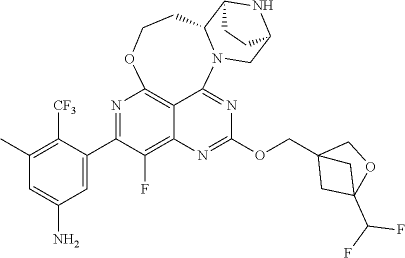 [SMILES: Cc1cc(N)cc(-c2nc3c4c(nc(OCC56COC(C(F)F)(C5)C6)nc4c2F)N2C[C@H]4CC[C@H](N4)[C@H]2CCO3)c1C(F)(F)F] |
| 8 | 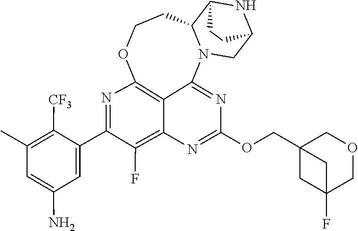 [SMILES: Cc1cc(N)cc(-c2nc3c4c(nc(OCC56COCC(F)(C5)C6)nc4c2F)N2C[C@H]4CC[C@H](N4)[C@H]2CCO3)c1C(F)(F)F] |
| 9 | 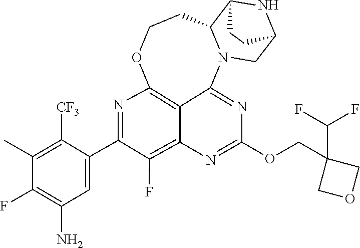 [SMILES: Cc1c(F)c(N)cc(-c2nc3c4c(nc(OCC5(C(F)F)COC5)nc4c2F)N2C[C@H]4CC[C@H](N4)[C@H]2CCO3)c1C(F)(F)F] |
| 10 | 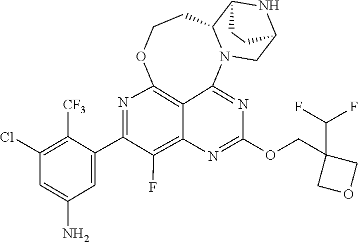 [SMILES: Nc1cc(Cl)c(C(F)(F)F)c(-c2nc3c4c(nc(OCC5(C(F)F)COC5)nc4c2F)N2C[C@H]4CC[C@H](N4)[C@H]2CCO3)c1] |
| 11 | 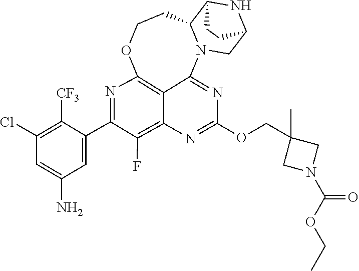 [SMILES: CCOC(=O)N1CC(C)(COc2nc3c4c(nc(-c5cc(N)cc(Cl)c5C(F)(F)F)c(F)c4n2)OCC[C@@H]2[C@@H]4CC[C@H](CN32)N4)C1] |
| 12 | 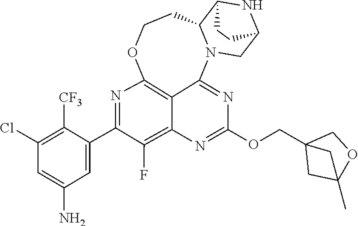 [SMILES: CC12CC(COc3nc4c5c(nc(-c6cc(N)cc(Cl)c6C(F)(F)F)c(F)c5n3)OCC[C@@H]3[C@@H]5CC[C@H](CN43)N5)(CO1)C2] |
| 13 | 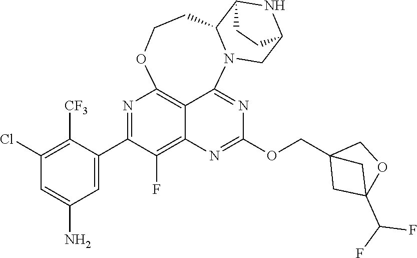 [SMILES: Nc1cc(Cl)c(C(F)(F)F)c(-c2nc3c4c(nc(OCC56COC(C(F)F)(C5)C6)nc4c2F)N2C[C@H]4CC[C@H](N4)[C@H]2CCO3)c1] |
| 14 | 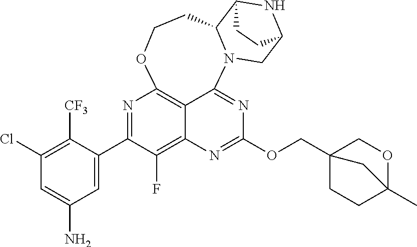 [SMILES: CC12CCC(COc3nc4c5c(nc(-c6cc(N)cc(Cl)c6C(F)(F)F)c(F)c5n3)OCC[C@@H]3[C@@H]5CC[C@H](CN43)N5)(CO1)C2] |
| 15 | 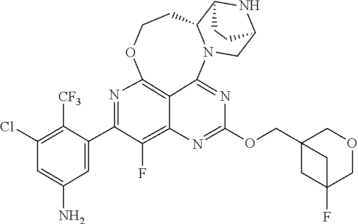 [SMILES: Nc1cc(Cl)c(C(F)(F)F)c(-c2nc3c4c(nc(OCC56COCC(F)(C5)C6)nc4c2F)N2C[C@H]4CC[C@H](N4)[C@H]2CCO3)c1] |
| 16 | 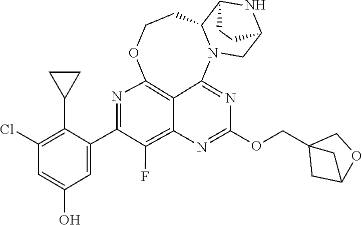 [SMILES: Oc1cc(Cl)c(C2CC2)c(-c2nc3c4c(nc(OCC56COC(C5)C6)nc4c2F)N2C[C@H]4CC[C@H](N4)[C@H]2CCO3)c1] |
| 17 | 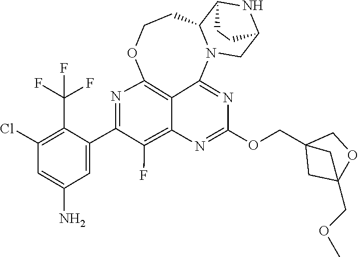 [SMILES: COCC12CC(COc3nc4c5c(nc(-c6cc(N)cc(Cl)c6C(F)(F)F)c(F)c5n3)OCC[C@@H]3[C@@H]5CC[C@H](CN43)N5)(CO1)C2] |
| 18 | 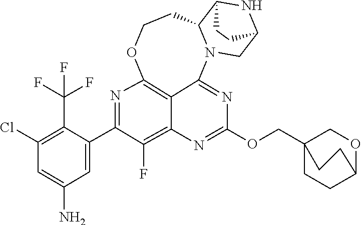 [SMILES: Nc1cc(Cl)c(C(F)(F)F)c(-c2nc3c4c(nc(OCC56CCC(CC5)OC6)nc4c2F)N2C[C@H]4CC[C@H](N4)[C@H]2CCO3)c1] |
| 19 | 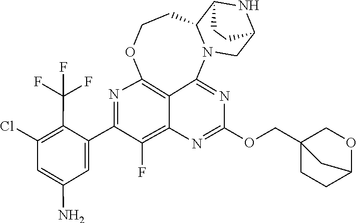 [SMILES: Nc1cc(Cl)c(C(F)(F)F)c(-c2nc3c4c(nc(OCC56CCC(C5)OC6)nc4c2F)N2C[C@H]4CC[C@H](N4)[C@H]2CCO3)c1] |
| 20 | 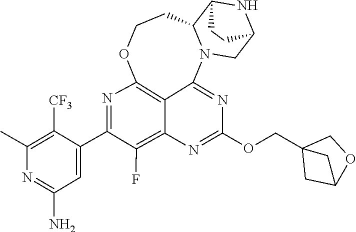 [SMILES: Cc1nc(N)cc(-c2nc3c4c(nc(OCC56COC(C5)C6)nc4c2F)N2C[C@H]4CC[C@H](N4)[C@H]2CCO3)c1C(F)(F)F] |
| 21 | 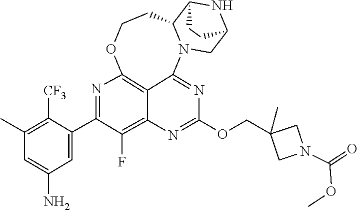 [SMILES: COC(=O)N1CC(C)(COc2nc3c4c(nc(-c5cc(N)cc(C)c5C(F)(F)F)c(F)c4n2)OCC[C@@H]2[C@@H]4CC[C@H](CN32)N4)C1] |
| 22 | 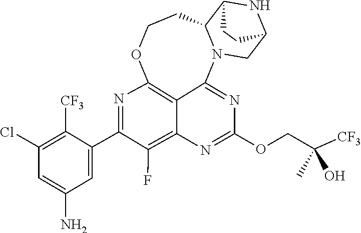 [SMILES: C[C@](O)(COc1nc2c3c(nc(-c4cc(N)cc(Cl)c4C(F)(F)F)c(F)c3n1)OCC[C@@H]1[C@@H]3CC[C@H](CN21)N3)C(F)(F)F] |
| 23 | 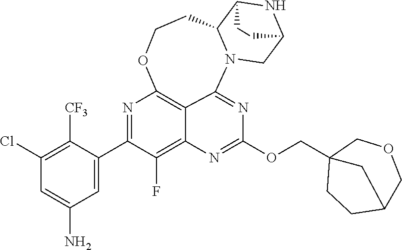 [SMILES: Nc1cc(Cl)c(C(F)(F)F)c(-c2nc3c4c(nc(OCC56CCC(COC5)C6)nc4c2F)N2C[C@H]4CC[C@H](N4)[C@H]2CCO3)c1] |
| 24 | 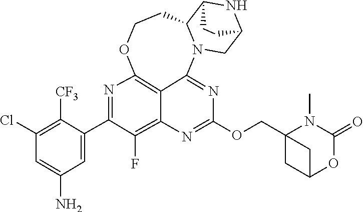 [SMILES: CN1C(=O)OC2CC1(COc1nc3c4c(nc(-c5cc(N)cc(Cl)c5C(F)(F)F)c(F)c4n1)OCC[C@@H]1[C@@H]4CC[C@H](CN31)N4)C2] |
| 25 | 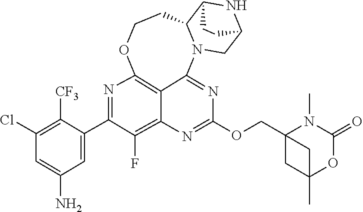 [SMILES: CN1C(=O)OC2(C)CC1(COc1nc3c4c(nc(-c5cc(N)cc(Cl)c5C(F)(F)F)c(F)c4n1)OCC[C@@H]1[C@@H]4CC[C@H](CN31)N4)C2] |
| 26 | 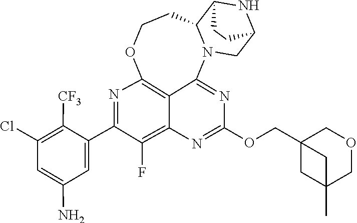 [SMILES: CC12COCC(COc3nc4c5c(nc(-c6cc(N)cc(Cl)c6C(F)(F)F)c(F)c5n3)OCC[C@@H]3[C@@H]5CC[C@H](CN43)N5)(C1)C2] |
| 27 | 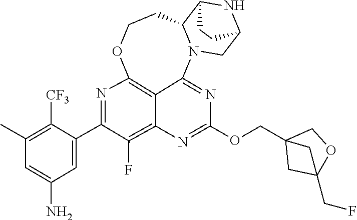 [SMILES: Cc1cc(N)cc(-c2nc3c4c(nc(OCC56COC(CF)(C5)C6)nc4c2F)N2C[C@H]4CC[C@H](N4)[C@H]2CCO3)c1C(F)(F)F] |
| 28 | 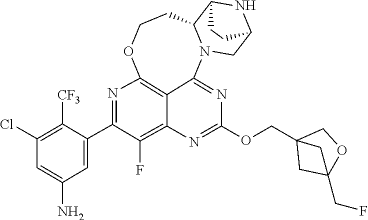 [SMILES: Nc1cc(Cl)c(C(F)(F)F)c(-c2nc3c4c(nc(OCC56COC(CF)(C5)C6)nc4c2F)N2C[C@H]4CC[C@H](N4)[C@H]2CCO3)c1] |
| 29 | 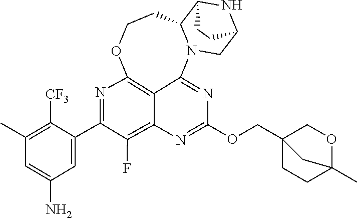 |
| 30 | 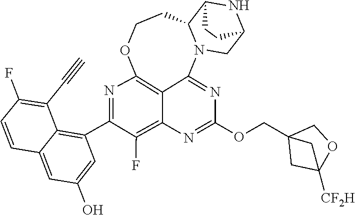 [SMILES: C#Cc1c(F)ccc2cc(O)cc(-c3nc4c5c(nc(OCC67COC(C(F)F)(C6)C7)nc5c3F)N3C[C@H]5CC[C@H](N5)[C@H]3CCO4)c12] |
| 31 | 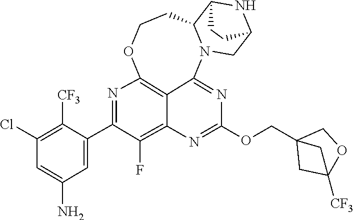 [SMILES: Nc1cc(Cl)c(C(F)(F)F)c(-c2nc3c4c(nc(OCC56COC(C(F)(F)F)(C5)C6)nc4c2F)N2C[C@H]4CC[C@H](N4)[C@H]2CCO3)c1] |
| 32 | 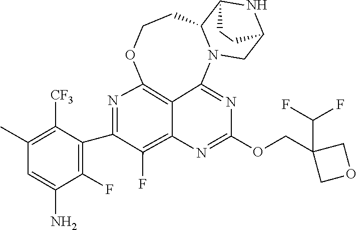 [SMILES: Cc1cc(N)c(F)c(-c2nc3c4c(nc(OCC5(C(F)F)COC5)nc4c2F)N2C[C@H]4CC[C@H](N4)[C@H]2CCO3)c1C(F)(F)F] |
| 33 | 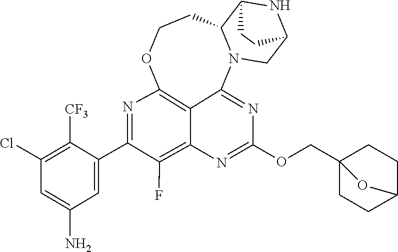 [SMILES: Nc1cc(Cl)c(C(F)(F)F)c(-c2nc3c4c(nc(OCC56CCC(CC5)O6)nc4c2F)N2C[C@H]4CC[C@H](N4)[C@H]2CCO3)c1] |
| 31 | 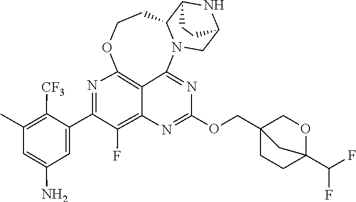 [SMILES: Cc1cc(N)cc(-c2nc3c4c(nc(OCC56CCC(C(F)F)(C5)OC6)nc4c2F)N2C[C@H]4CC[C@H](N4)[C@H]2CCO3)c1C(F)(F)F] |
| 32 | 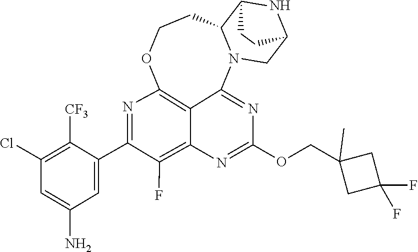 [SMILES: CC1(COc2nc3c4c(nc(-c5cc(N)cc(Cl)c5C(F)(F)F)c(F)c4n2)OCC[C@@H]2[C@@H]4CC[C@H](CN32)N4)CC(F)(F)C1] |
| 33 | 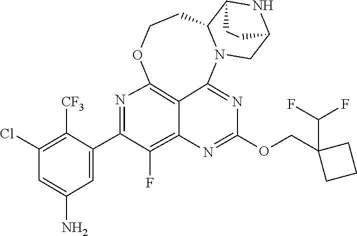 [SMILES: Nc1cc(Cl)c(C(F)(F)F)c(-c2nc3c4c(nc(OCC5(C(F)F)CCC5)nc4c2F)N2C[C@H]4CC[C@H](N4)[C@H]2CCO3)c1] |
| 34 | 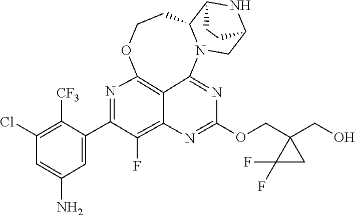 [SMILES: Nc1cc(Cl)c(C(F)(F)F)c(-c2nc3c4c(nc(OCC5(CO)CC5(F)F)nc4c2F)N2C[C@H]4CC[C@H](N4)[C@H]2CCO3)c1] |
| 35 | 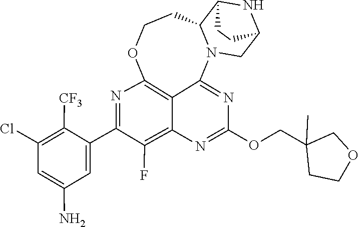 [SMILES: CC1(COc2nc3c4c(nc(-c5cc(N)cc(Cl)c5C(F)(F)F)c(F)c4n2)OCC[C@@H]2[C@@H]4CC[C@H](CN32)N4)CCOC1] |
| 36 | 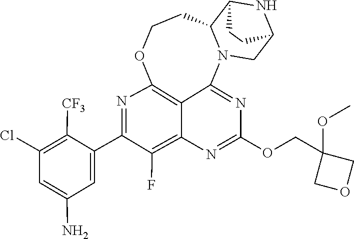 [SMILES: COC1(COc2nc3c4c(nc(-c5cc(N)cc(Cl)c5C(F)(F)F)c(F)c4n2)OCC[C@@H]2[C@@H]4CC[C@H](CN32)N4)COC1] |
| 37 | 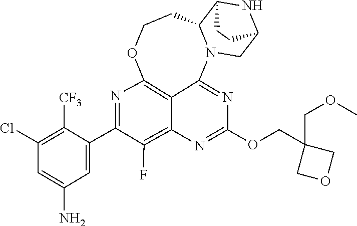 [SMILES: COCC1(COc2nc3c4c(nc(-c5cc(N)cc(Cl)c5C(F)(F)F)c(F)c4n2)OCC[C@@H]2[C@@H]4CC[C@H](CN32)N4)COC1] |
| 38 | 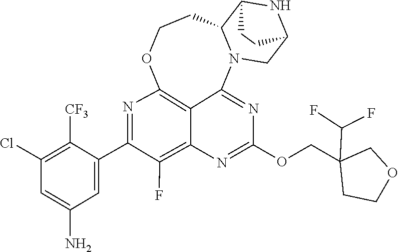 [SMILES: Nc1cc(Cl)c(C(F)(F)F)c(-c2nc3c4c(nc(OCC5(C(F)F)CCOC5)nc4c2F)N2C[C@H]4CC[C@H](N4)[C@H]2CCO3)c1] |
| 39 | 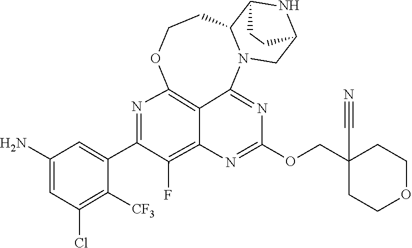 [SMILES: N#CC1(COc2nc3c4c(nc(-c5cc(N)cc(Cl)c5C(F)(F)F)c(F)c4n2)OCC[C@@H]2[C@@H]4CC[C@H](CN32)N4)CCOCC1] |
| 40 | 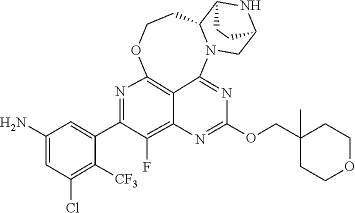 [SMILES: CC1(COc2nc3c4c(nc(-c5cc(N)cc(Cl)c5C(F)(F)F)c(F)c4n2)OCC[C@@H]2[C@@H]4CC[C@H](CN32)N4)CCOCC1] |
| 41 | 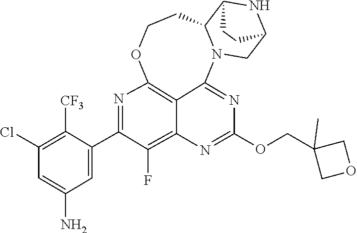 [SMILES: CC1(COc2nc3c4c(nc(-c5cc(N)cc(Cl)c5C(F)(F)F)c(F)c4n2)OCC[C@@H]2[C@@H]4CC[C@H](CN32)N4)COC1] |
| 42 | 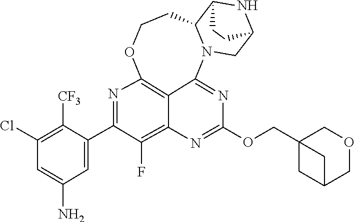 [SMILES: Nc1cc(Cl)c(C(F)(F)F)c(-c2nc3c4c(nc(OCC56COCC(C5)C6)nc4c2F)N2C[C@H]4CC[C@H](N4)[C@H]2CCO3)c1] |
| 43 | 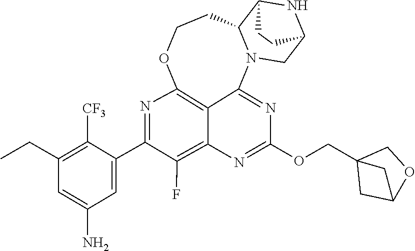 [SMILES: CCc1cc(N)cc(-c2nc3c4c(nc(OCC56COC(C5)C6)nc4c2F)N2C[C@H]4CC[C@H](N4)[C@H]2CCO3)c1C(F)(F)F] |

In some embodiments, L¹ is —O—.

In some embodiments, the compound of formula (I) is a compound of formula (IIa):

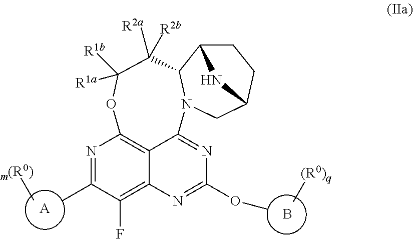

wherein the variables are defined above.

In some embodiments, moiety B is substituted or unsubstituted cycloalkyl, or substituted or unsubstituted heterocyclyl containing 1-3 heteroatoms independently selected from N or O.

In some embodiments, moiety B is substituted or unsubstituted 4- to 6-membered cycloalkyl, or substituted or unsubstituted heterocyclyl containing one oxygen atom.

In some embodiments, moiety B is substituted or unsubstituted tetrahydro-2H-pyran-yl or substituted or unsubstituted furanyl; preferably tetrahydro-2H-pyran-yl or furanyl, wherein said each of tetrahydro-2H-pyran-yl or furanyl is optionally substituted with C₁₋₄alkylamino or heterocyclyl containing one or more nitrogen atoms; more preferably tetrahydro-2H-pyran-yl substituted with dimethylamino, or furanyl substituted with dimethylamino.

In some embodiments, moiety B is

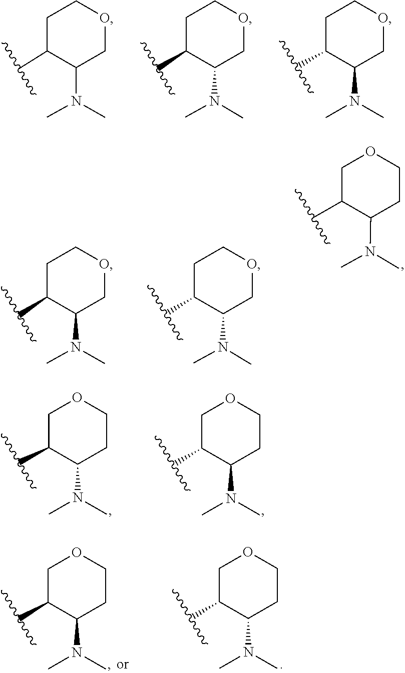

In some embodiments, moiety B is

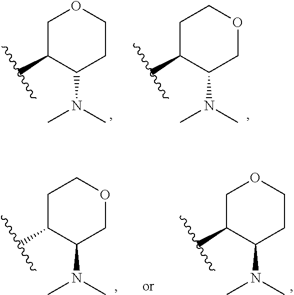

In some embodiments, moiety B is

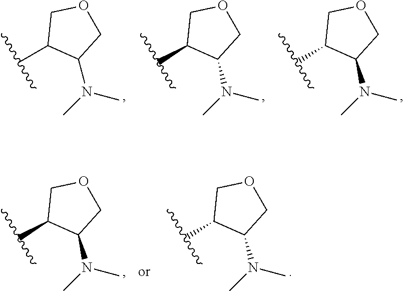

In some embodiments, moiety B is,

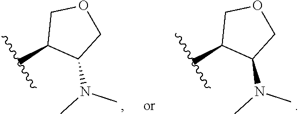

In some embodiments, moiety B is substituted or unsubstituted cyclobutyl, substituted or unsubstituted cyclopentyl, or substituted or unsubstituted cycloheptyl; preferably substituted or unsubstituted 2-dimethylamino-cyclobutyl, substituted or unsubstituted 2-heterocyclyl-cyclobutyl, substituted or unsubstituted 2-dimethylamino-cyclopentyl, substituted or unsubstituted 2-heterocyclyl-cyclopentyl, substituted or unsubstituted 2-dimethylamino-cycloheptyl, substituted or unsubstituted 2-heterocyclyl-cycloheptyl, wherein said heterocyclyl is the heterocyclyl containing one or more nitrogen atoms; more preferably 2-dimethylamino-cyclobutyl, 2-dimethylamino-cyclopentyl, or 2-dimethylamino-cycloheptyl, wherein said each of 2-dimethylamino-cyclobutyl, 2-dimethylamino-cyclopentyl and 2-dimethylamino-cycloheptyl is optionally substituted with H, F, methoxy, methyl, methyl-d3, methoxy-d3, CF₂H, or CF₃.

In some embodiments, moiety B is

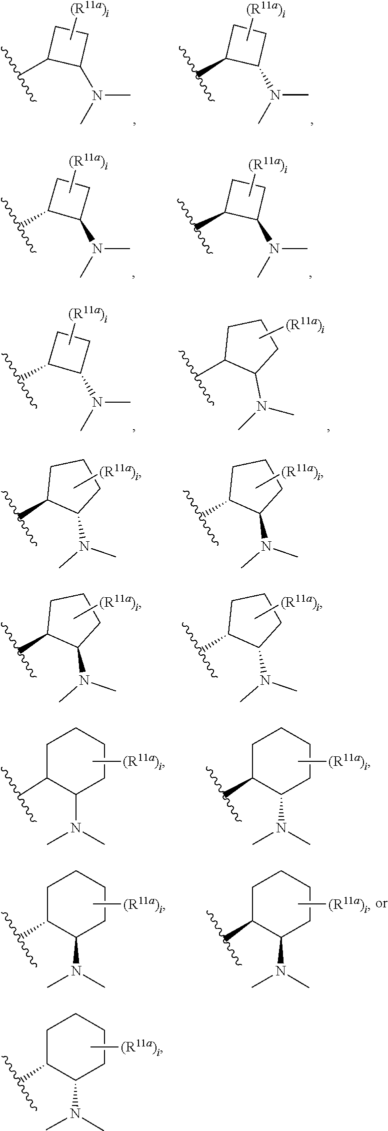

wherein R^{11a} is H, F, methoxy, methyl, methyl-d3, methoxy-d3, CF₂H, or CF₃; and
i is 0, 1, 2, 3, or 4. In one embodiment, i is 0. In one embodiment, i is 1. In one embodiment, i is 2. In one embodiment, i is 3. In one embodiment, i is 4.

In some embodiments, moiety B is

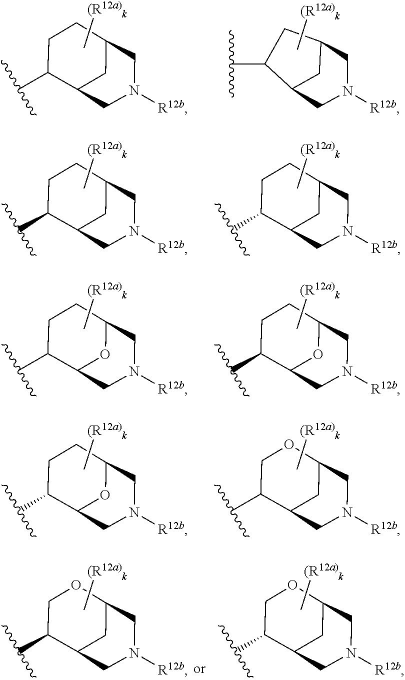

wherein
R^{12a} is H, F, methoxy, methyl, methyl-d3, methoxy-d3, difluoromethyl, or trifluoromethyl;
R^{12b} is C₁₋₃alkyl optionally substituted with one or more substituents selected from methyl, methyl-d3, F, methoxy, or alkenyl; and
k is 0, 1, or 2.

In some embodiments, moiety B is

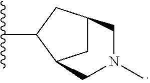 [SMILES: *C1C[C@@H]2C[C@H]1CN(C)C2]

In one embodiment, k is 0. In one embodiment, k is 1. In one embodiment k is 2.

In some embodiments,

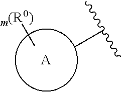

is

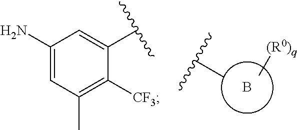

is

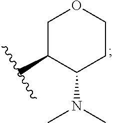 [SMILES: *[C@H]1COCC[C@@H]1N(C)C]

and all of R^{1a}, R^{1b}, R^{2a} and R^{2b} are H.

In some embodiments, L¹ is a —O—C(R^{3a})(R^{3b})—, wherein said each of R^{3a} and R^{3b} is, independently, H, F, unsubstituted or substituted C₁₋₂alkyl.

In some embodiments, the compound of formula (I) is a compound of formula (IIb):

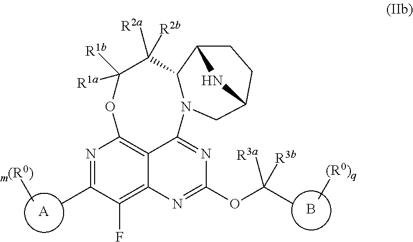

wherein
each of R^{3a} and R^{3b}, is, independently, H, F, unsubstituted or substituted C₁₋₂alkyl; and
the other variables are defined above.

In some embodiments, the compound of formula (IIb) is a compound of formula (IIIb):

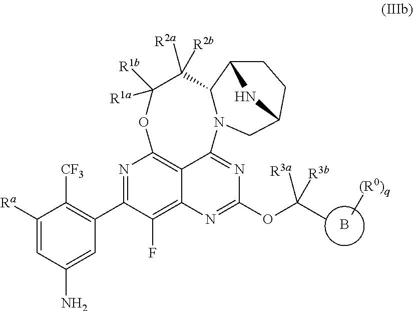

wherein
R^{a} is methyl, methyl-d3, or Cl; and
the other variables are defined above.

In some embodiments, R^{3a} is H, methyl, methyl-d3, difluoromethyl, or trifluoromethyl; and R^{3b} is H.

In some embodiments, both R^{3a} and R^{3b} are H.

In some embodiments, both R^{3a} and R^{3b} are deuterium.

In some embodiments, R^{3a} is methyl; R^{3b} is H.

In some embodiments, the carbon atom which R^{3a} and R^{3b} connect to is R-configuration, provided R^{3a} and R^{3b} are different substituents.

In some embodiments, the carbon atom which R^{3a} and R^{3b} connect to is S-configuration, provided R^{3a} and R^{3b} are different substituents.

In some embodiments, the compound of formula (IIb) is a compound of formula (IIIa):

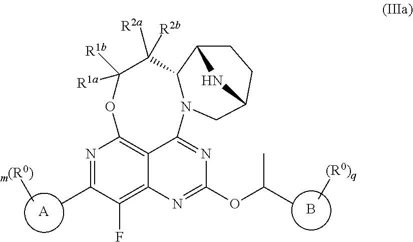

the variables are defined above.

In some embodiments, the compound of formula (IIIa) is a compound of formula (IVa):

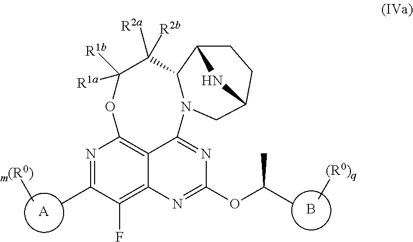

the variables are defined above.

In some embodiments, moiety B is substituted or unsubstituted C₃₋₆cycloalkyl, or substituted or unsubstituted 4- to 6-membered heterocyclyl containing one or more hetero atoms; preferably cyclopropyl or substituted or unsubstituted heterocyclyl containing one or more heteroatoms selected from N or O, wherein said cyclopropyl is optionally substituted with one or more substituents selected from halogen, or substituted or unsubstituted alkyl.

In some embodiments, moiety B is cyclopropyl optionally substituted with one or more substituents selected from halogen, or substituted or unsubstituted alkyl.

In some embodiments, moiety B is cyclopropyl optionally substituted with one or more substituents selected from halogen, or alkyl, wherein said alkyl is optionally substituted with substituted or unsubstituted 4- to 10-membered heterocyclyl.

In some embodiments, moiety B is

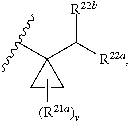

wherein
each of R^{21a} is, independently, H or halogen;
each of R^{22a} and R^{22b} is, independently, H, substituted or unsubstituted alkyl, substituted or unsubstituted amino or substituted or unsubstituted heterocyclyl, or R^{22a} and R^{22b}, together with the atom which R^{22a} and R^{22b} connect with, form a substituted or unsubstituted heterocyclyl; and
v is 0, 1, 2, 3, or 4.

In one embodiment, v is 0. In one embodiment, v is 1. In one embodiment, v is 2. In one embodiment, v is 3. In one embodiment, v is 4.

In some embodiments, moiety B is

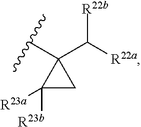

wherein
each of R^{23a} and R^{23b} is, independently, H or halogen;
each of R^{22a} and R^{22b} is, independently, H, substituted or unsubstituted alkyl, substituted or unsubstituted amino or substituted or unsubstituted heterocyclyl, or R^{22a} and R^{22b}, together with the atom which R^{22a} and R^{22b} connect with, form a substituted or unsubstituted heterocyclyl.

In some embodiments, the carbon atom of cyclopropyl attached to the branch containing R^{22a} and R^{22b} is R-configuration.

In some embodiments the carbon atom of cyclopropyl attached to the branch containing R^{22a} and R^{22b} is S-configuration.

In some embodiments, moiety B is

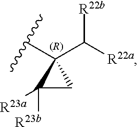

wherein
each of R^{23a} and R^{23b} is, independently, H or halogen;
each of R^{22a} and R^{22b} is, independently, H, substituted or unsubstituted alkyl, substituted or unsubstituted amino or substituted or unsubstituted heterocyclyl, or R^{22a} and R^{22b}, together with the atom which R^{22a} and R^{22b} connect with, form a substituted or unsubstituted heterocyclyl.

In some embodiments, moiety B is

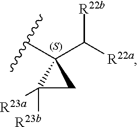

wherein
each of R^{23a} and R^{23b} is, independently, H or halogen;
each of R^{22a} and R^{22b} is, independently, H, substituted or unsubstituted alkyl, substituted or unsubstituted amino or substituted or unsubstituted heterocyclyl, or R^{22a} and R^{22b}, together with the atom which R^{22a} and R^{22b} connect with, form a substituted or unsubstituted heterocyclyl.

In some embodiments, each of R^{22a} and R^{22b} is, independently, H, substituted or unsubstituted amino, or substituted or unsubstituted C₁₋₃alkyl; preferably each of R^{22a} and R^{22b} is, independently, H, methyl, or dimethylamino.

In some embodiments, moiety B is

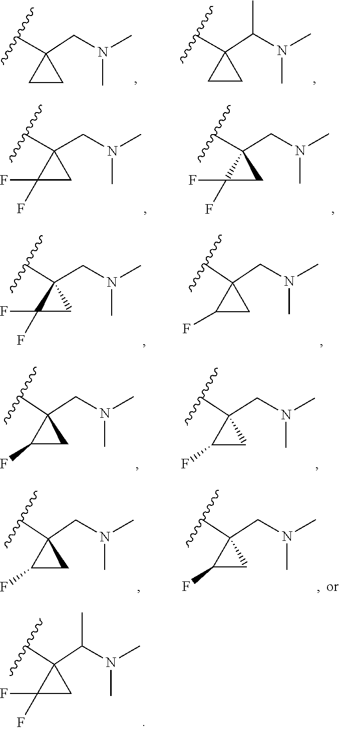

In some embodiments, moiety B is

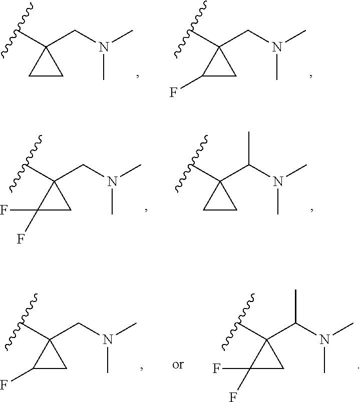

In some embodiments, moiety B is

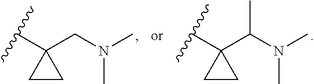

In some embodiments, moiety B is

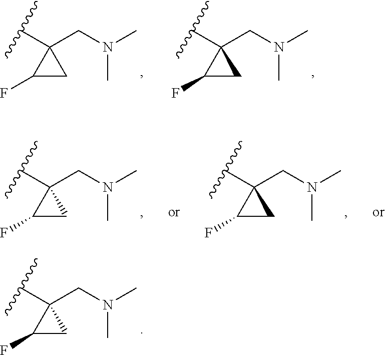

In some embodiments, moiety B is

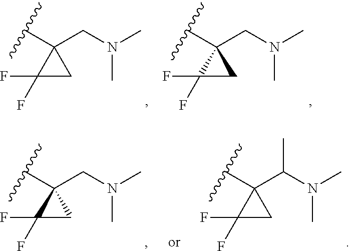

In some embodiments, R^{22b} is H or methyl and R^{22a} is substituted or unsubstituted heterocyclyl; preferably R^{22b} is H or methyl and R^{22a} is substituted or unsubstituted azetidinyl, substituted or unsubstituted pyrrolidinyl, substituted or unsubstituted piperidinyl, substituted or unsubstituted 3-azabicyclo[3.1.0]hexanyl, substituted or unsubstituted 2,5-dihydro-1H-pyrrolyl, substituted or unsubstituted 6-azaspiro[2.5]octanyl, substituted or unsubstituted 1,2,3,6-tetrahydropyridinyl, substituted or unsubstituted morpholinyl, substituted or unsubstituted 2-oxa-6-azaspiro[3.3]heptanyl, substituted or unsubstituted (1R,4R)-2-oxa-5-azabicyclo[2.2.1]heptanyl, substituted or unsubstituted (1S,4S)-2-oxa-5-azabicyclo[2.2.1]heptanyl; more preferably R^{22b} is H or methyl and R^{22a} is substituted or unsubstituted azetidine-1-yl, substituted or unsubstituted pyrrolidine-1-yl, substituted or unsubstituted piperidin-1-yl, substituted or unsubstituted 2,5-dihydro-1H-pyrrol-1-yl, substituted or unsubstituted 6-azaspiro[2.5]octan-6-yl, substituted or unsubstituted 1,2,3,6-tetrahydropyridin-1-yl, substituted or unsubstituted morpholin-4-yl, substituted or unsubstituted 2-oxa-6-azaspiro[3.3]heptan-6-yl, substituted or unsubstituted (1R,4R)-2-oxa-5-azabicyclo[2.2.1]heptan-5-yl, substituted or unsubstituted (1S,4S)-2-oxa-5-azabicyclo[2.2.1]heptan-5-yl, 2-azaspiro[3.3]heptane-2-yl, substituted or unsubstituted 2-azabicyclo[2.2.1]heptan-2-yl, substituted or unsubstituted 3-azabicyclo[3.1.1]heptan-3-yl, substituted or unsubstituted 2-oxa-6-azaspiro[3.4]octan-6-yl, substituted or unsubstituted 2-oxa-7-azaspiro[4.4]nonan-7-yl, substituted or unsubstituted 7-oxa-1-azaspiro[4.4]nonan-1-yl, or substituted or unsubstituted 7-azaspiro[3.5]nonan-7-yl.

In some embodiments, moiety B is

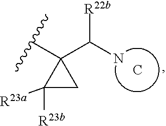

wherein ring C is substituted or unsubstituted 4- to 10-membered heterocyclyl optionally containing one or more additional heteroatom selected from N, O, or S. In some embodiments, ring C is substituted or unsubstituted 4- to 7-membered heterocycly optionally containing one or more additional heteroatom selected from N, O, or S. In some embodiments, said heterocycly is monocycle system. In some embodiments, said heterocycly is bridged ring systems. In some embodiments, said heterocycly is fused ring systems In some embodiments, said heterocycly is spirocyclic ring systems. In some embodiments, R^{22b} is H or methyl. In some embodiments, R^{22b} is H. In some embodiments, R^{22b} is methyl.

In some embodiments, ring C is optionally substituted with one or more substituents, wherein the substituent is halogen, hydroxy, substituted or unsubstituted C₁₋₃alkoxy, substituted or unsubstituted C₁₋₃alkyl, or substituted or unsubstituted C₃₋₅cycloalkyl, or one pair of the substituents, together with the atom(s) they are connected with, form a substituted or unsubstituted alkenyl, substituted or unsubstituted cycloalkyl, substituted or unsubstituted heterocyclyl.

In some embodiments, ring C is optionally substituted with one or more substituents, wherein the substituent is H, F, hydroxy, cyano, methoxy, methyl, methyl-d3, ethyl, fluoromethyl, difluoromethyl, trifluoromethyl, hydroxymethyl, difluoromethoxy, cyclopropyl, or oxetanyl.

In some embodiments, ring C is optionally substituted with one or more substituents, wherein one pair of the substituents, together with the atoms they are connected with, form a substituted or unsubstituted alkenyl, wherein said alkenyl is in ring C (e.g. 2,5-dihydro-1H-pyrrol-1-yl).

In some embodiments, moiety B is

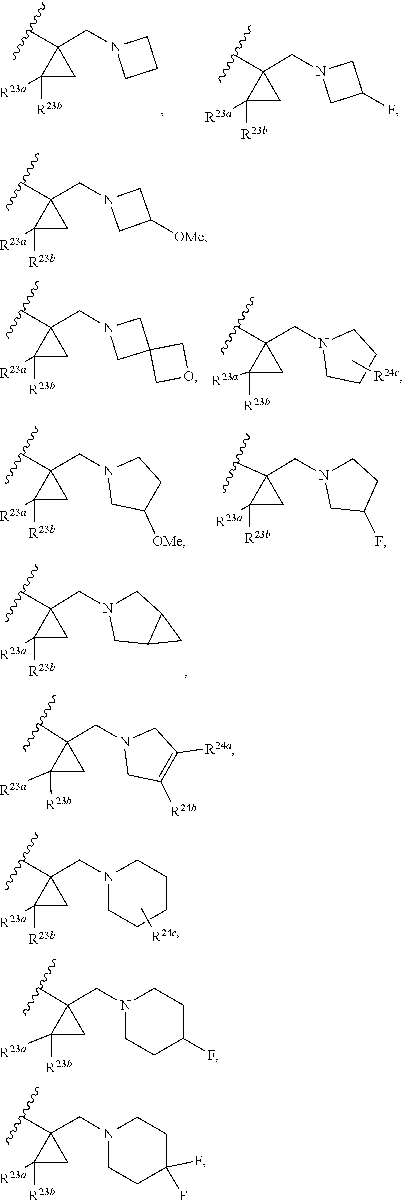

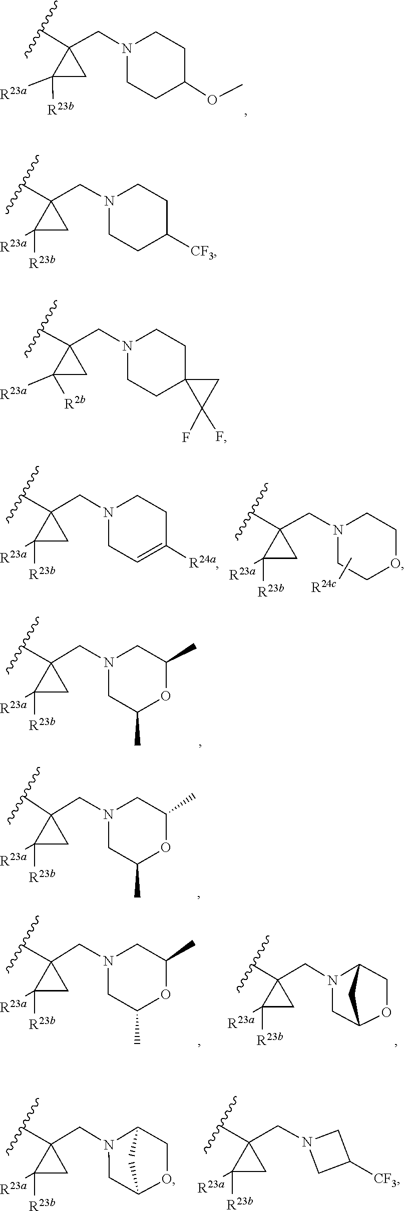

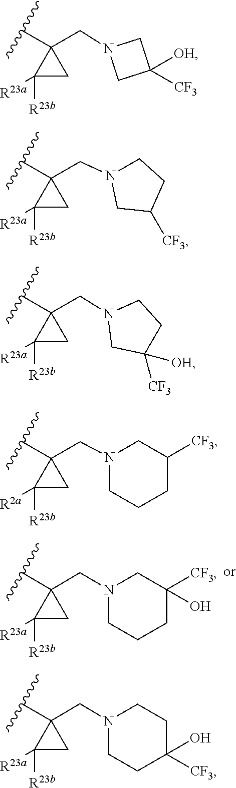

wherein each of R^{23a} and R^{23b} is, independently, H or F; each of R^{24a} and R^{24b} is, independently, H, methyl, ethyl, methyl-d3, F, or Cl; and R^{24c} is H, F, methoxy, methyl, methyl-d3, ethyl, difluoromethyl, or trifluoromethyl.

In some embodiments, moiety B is

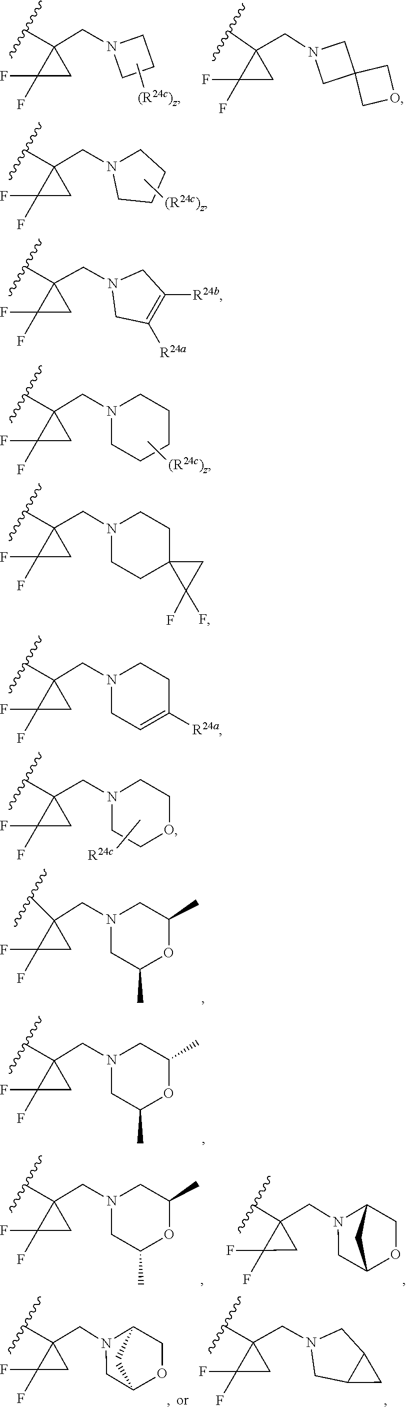

wherein each of R^{24a} and R^{24b} is, independently, H, methyl, ethyl, methyl-d3, F, or Cl; R^{24c} is H, F, methoxy, methyl, methyl-d3, ethyl, difluoromethyl, or trifluoromethyl; and z is 0, 1, or 2.

In some embodiments, moiety B is

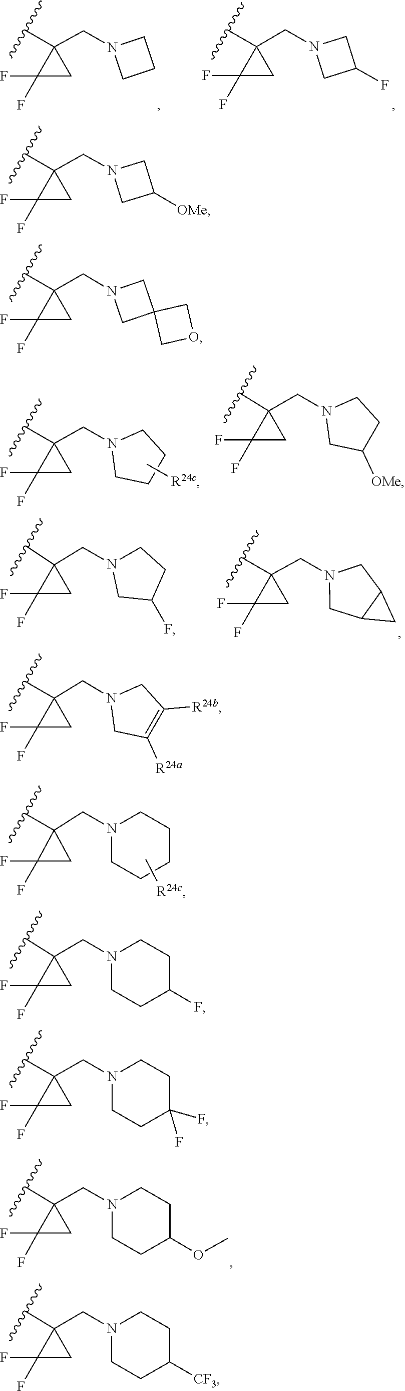

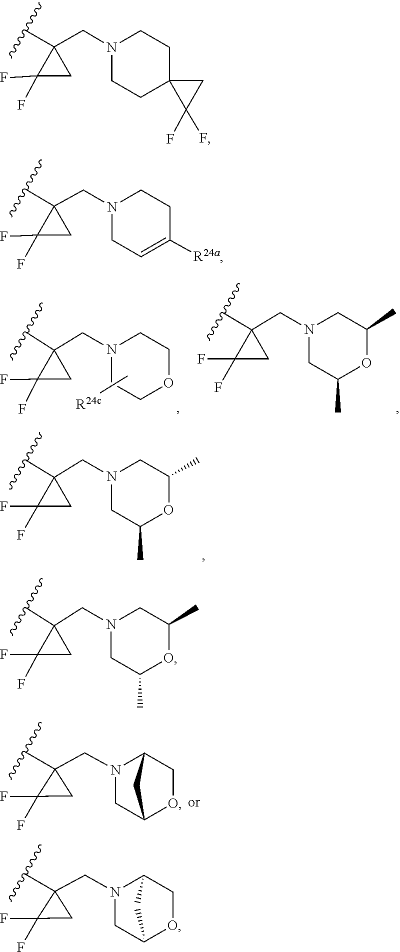

wherein each of R^{24a} and R^{24b} is, independently, H, methyl, ethyl, methyl-d3, F, or Cl; and R^{24c} is H, F, methoxy, methyl, methyl-d3, ethyl, difluoromethyl, or trifluoromethyl. In some embodiments, moiety B is

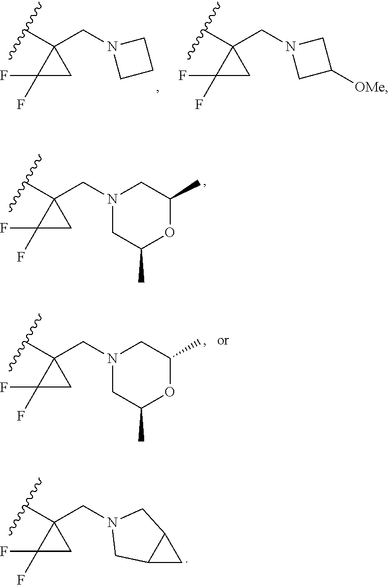

In some embodiments, moiety B is

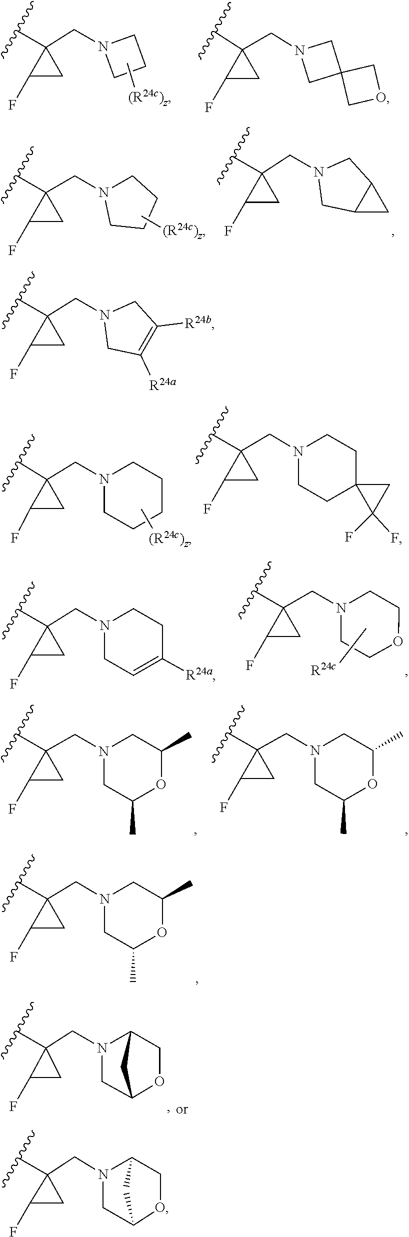

wherein each of R^{24a} and R^{24b} is, independently, H, methyl, ethyl, methyl-d3, F, or Cl; R^{24c} is H, F, methoxy, methyl, methyl-d3, ethyl, difluoromethyl, or trifluoromethyl; and z is 0, 1, or 2.

In some embodiments, moiety B is

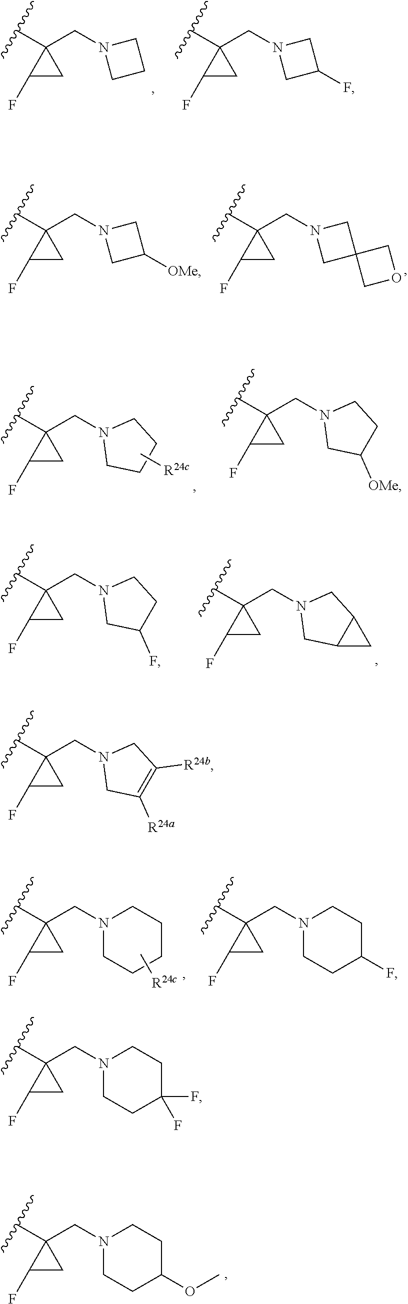

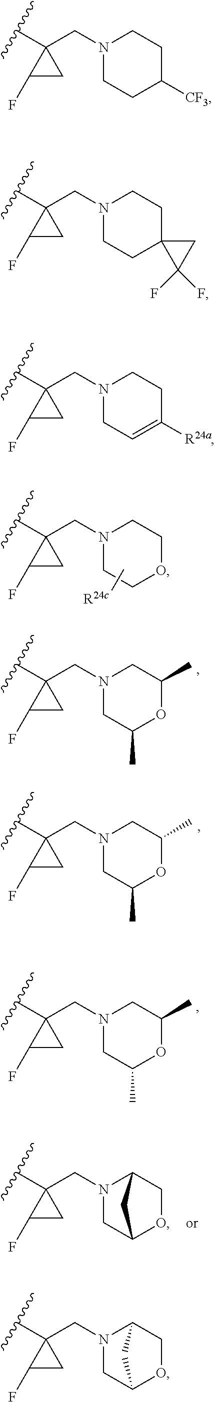

wherein each of R^{24a} and R^{24b} is, independently, H, methyl, ethyl, methyl-d3, F, or Cl; and R^{24c} is H, F, methoxy, methyl, methyl-d3, ethyl, difluoromethyl, or trifluoromethyl.

In some embodiments, moiety B is

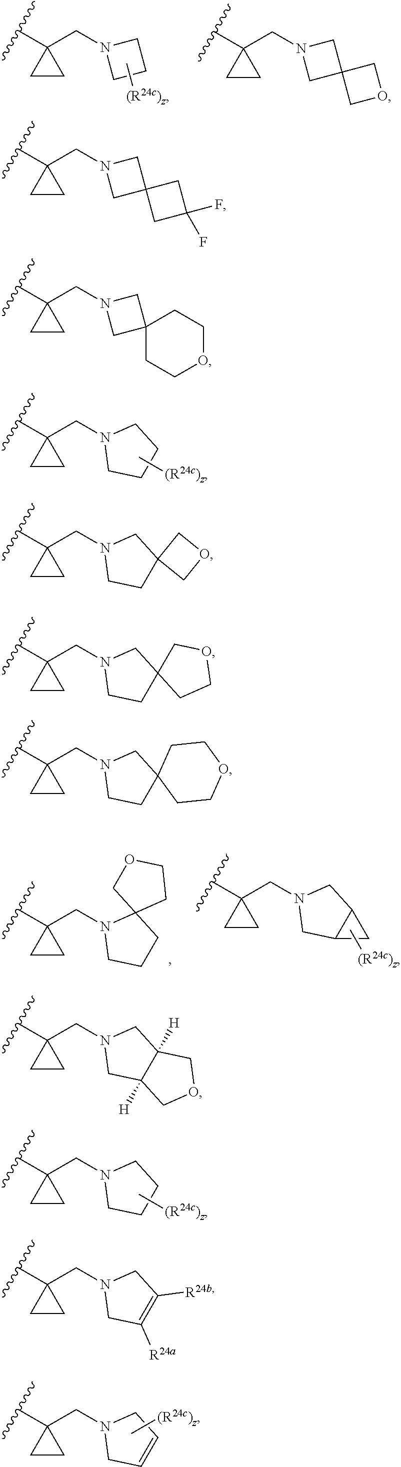

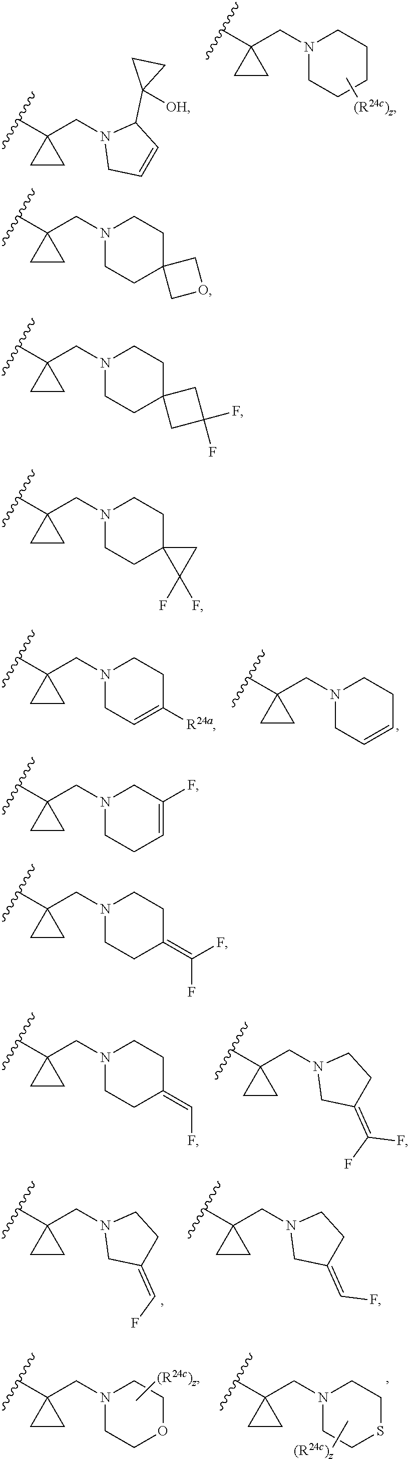

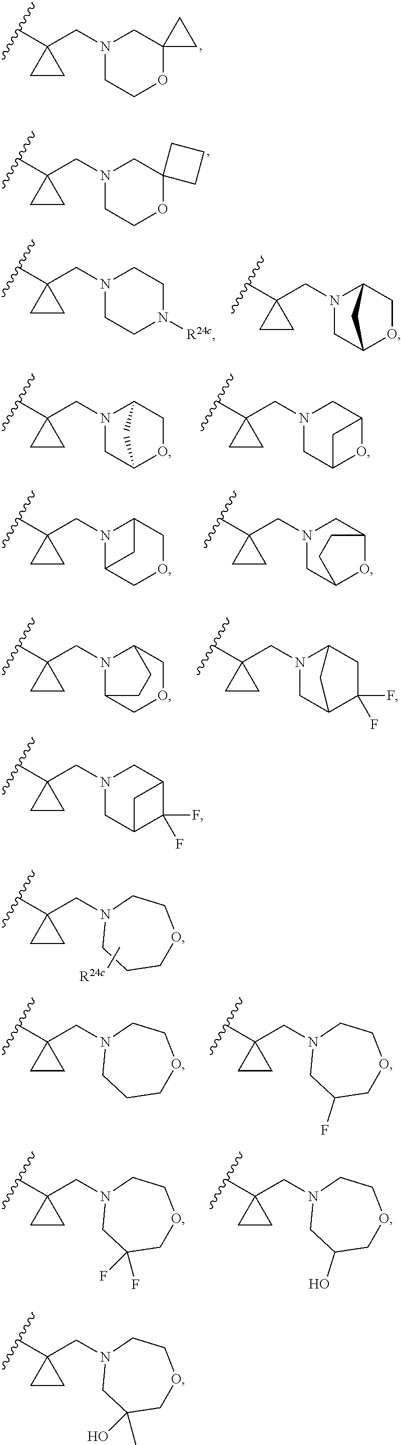

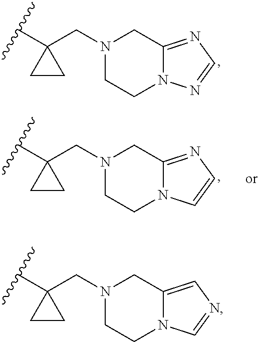 [SMILES: *C1(CN2CCn3ncnc3C2)CC1 ; *C1(CN2CCn3ccnc3C2)CC1 ; *C1(CN2CCn3cncc3C2)CC1]

wherein each of R^{24a} and R^{24b} is, independently, H, methyl, ethyl, methyl-d3, F, or Cl; and R^{24c} is H, F, hydroxy, cyano, methoxy, methyl, methyl-d3, ethyl, fluoromethyl, difluoromethyl, trifluoromethyl, hydroxymethyl, difluoromethoxy, cyclopropyl, or oxetanyl.

In some embodiments, moiety B is

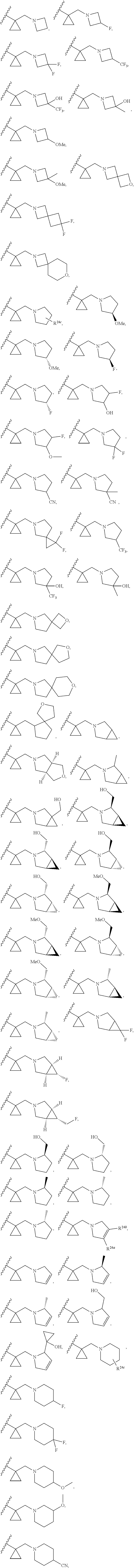

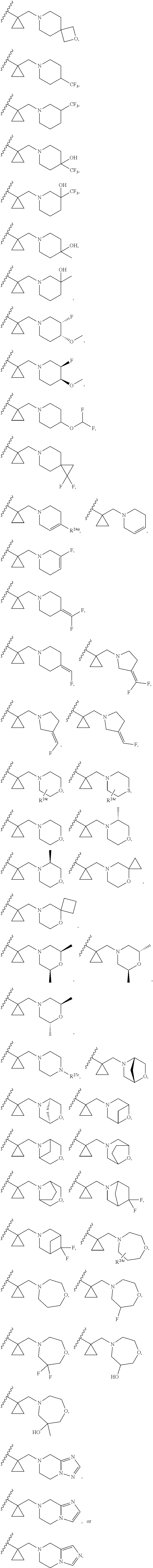

wherein each of R^{24a} and R^{24b} is, independently, H, methyl, ethyl, methyl-d3, F, or Cl; and R^{24c} is H, F, methoxy, methyl, methyl-d3, ethyl, difluoromethyl, or trifluoromethyl.

In some embodiments, moiety B is

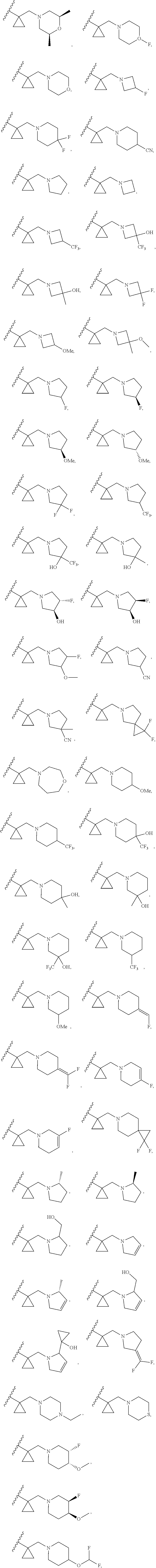

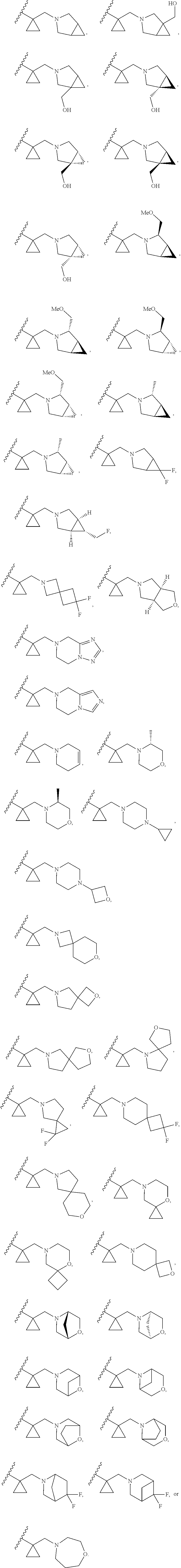

In some embodiments, moiety B is

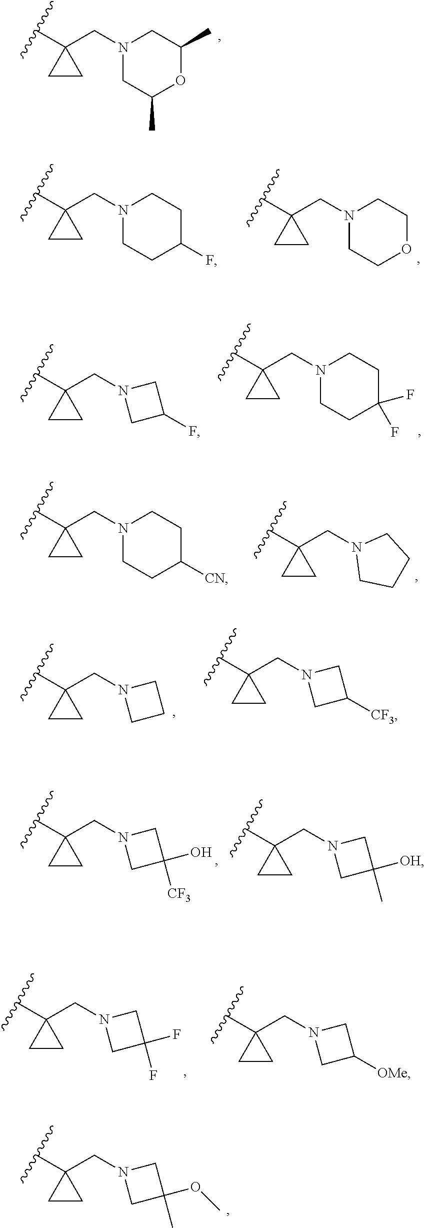

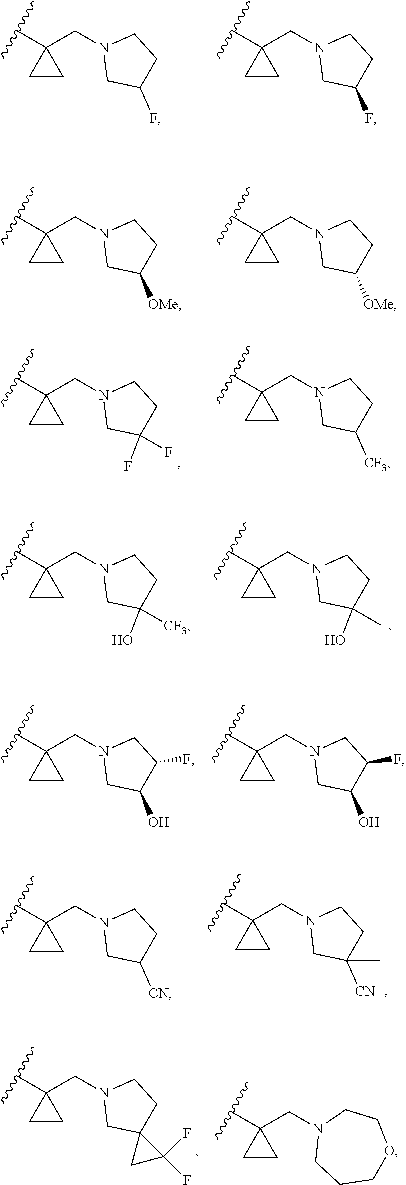

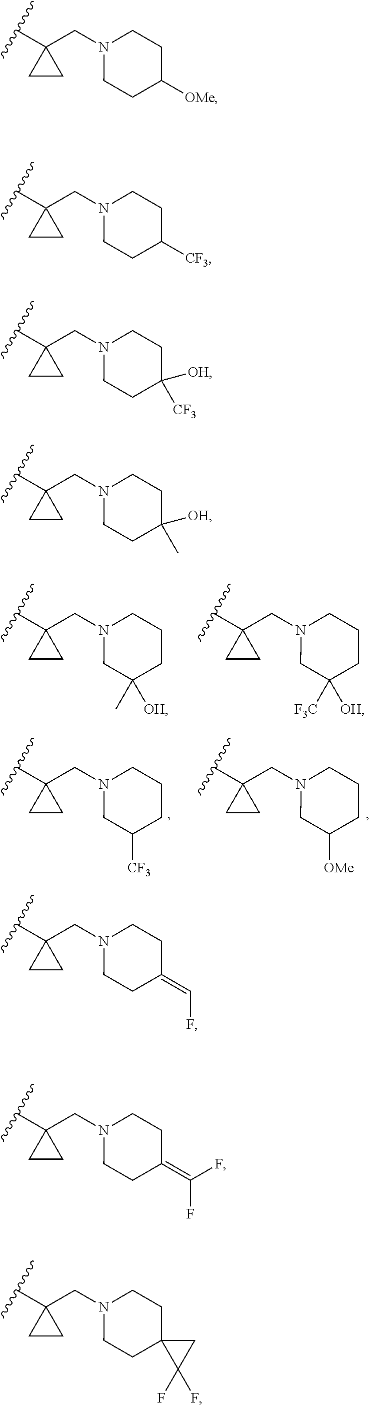

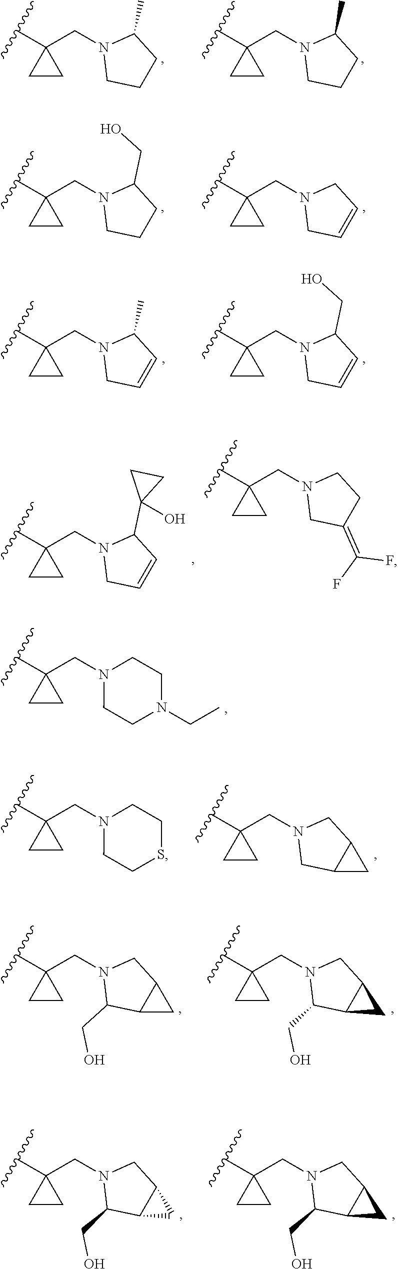

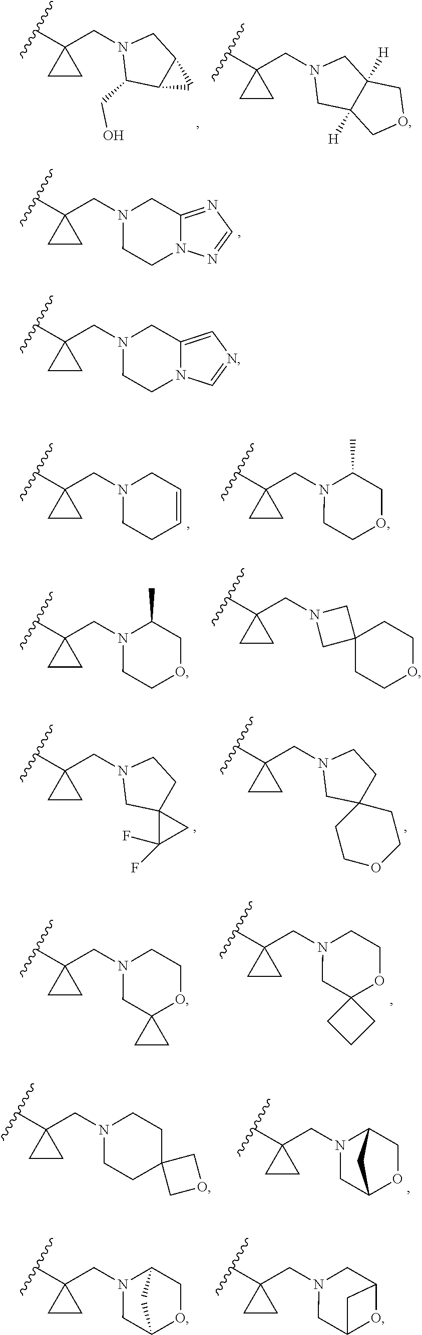

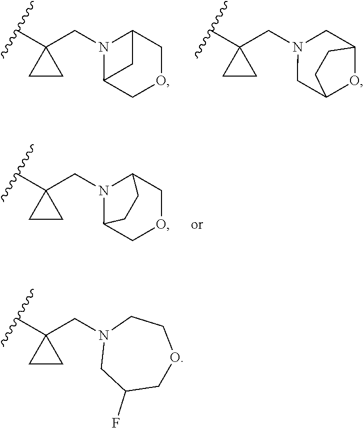 [SMILES: *C1(CN2C3COCC2C3)CC1 ; *C1(CN2CC3CCC(C2)O3)CC1 ; *C1(CN2C3CCC2COC3)CC1 ; *C1(CN2CCOCC(F)C2)CC1]

In some embodiments, moiety B is

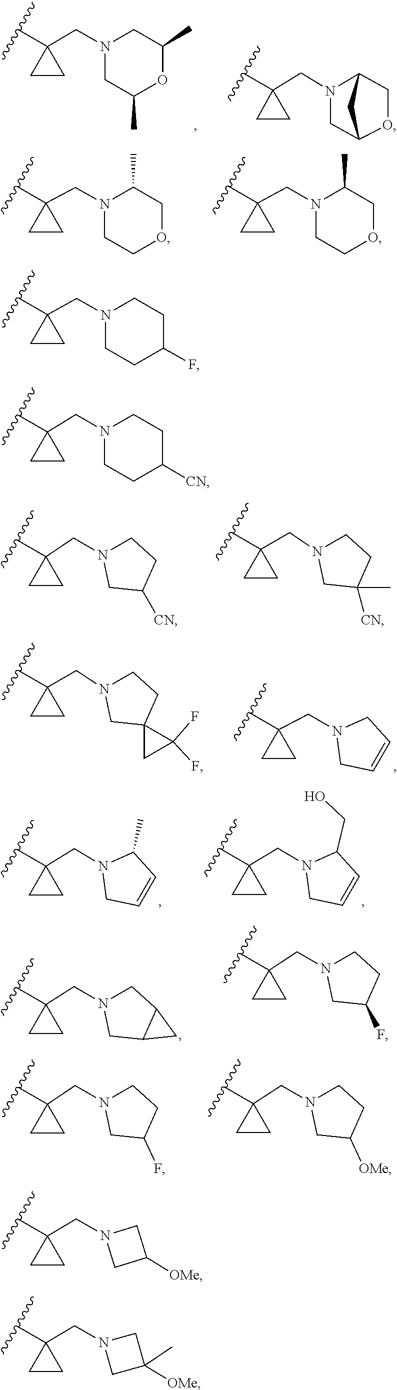

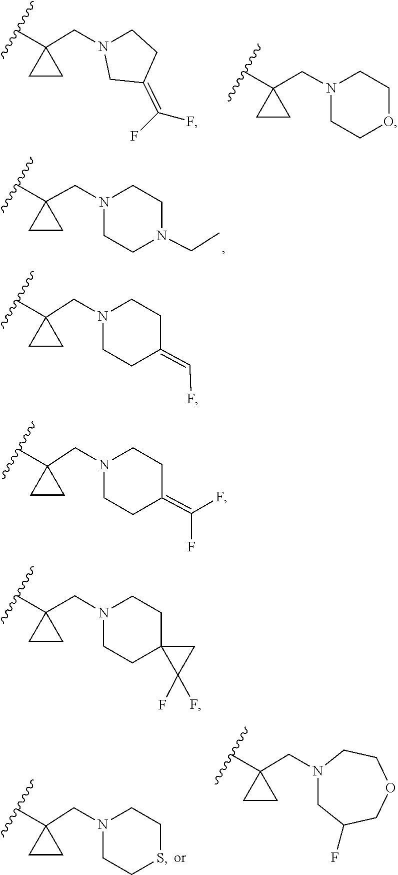

In some embodiments, R^{22a} and R^{22b}, together with the atom which R^{22a} and R^{22b} connect with, form a substituted or unsubstituted 5- to 6-membered heterocyclyl; In some embodiments, R^{22a} and R^{22b}, together with the atom which R^{22a} and R^{22b} connect with, form a substituted or unsubstituted pyrrolidinyl, substituted or unsubstituted piperidinyl, or substituted or unsubstituted morpholinyl; In some embodiments, R^{22a} and R^{22b}, together with the atom which R^{22a} and R^{22b} connect with, form a pyrrolidinyl, piperidinyl, or morpholinyl, wherein said each of pyrrolidinyl, piperidinyl, and morpholinyl is optionally substituted with H, halogen, substituted or unsubstituted alkoxy, or substituted or unsubstituted alkyl.

In some embodiments, moiety B is

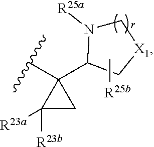

wherein R^{25a} is C₁₋₃alkyl optionally substituted with one or more substituents selected from methyl, methyl-d3, F or alkenyl;
R^{25b} is H, F, methoxy, methyl, difluoromethyl, or trifluoromethyl;
X¹ is O or C; and
r is 0, 1 or 2.

In some embodiments, moiety B is

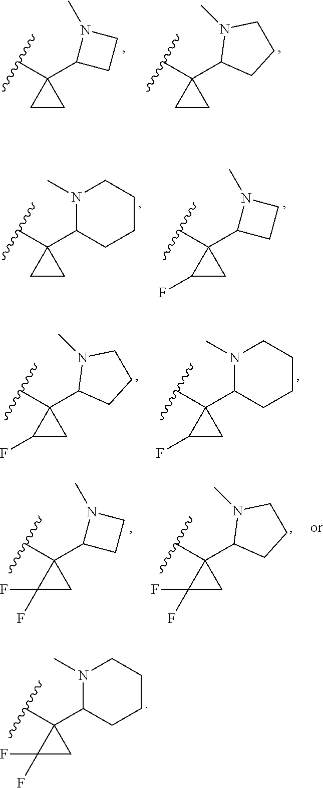

In some embodiments, moiety B is

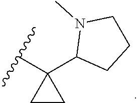 [SMILES: *C1(C2CCCN2C)CC1]

In some embodiments, moiety B is substituted or unsubstituted 4- to 6-membered heterocyclyl containing one or two heteroatoms selected from N or O.

In some embodiments, moiety B is substituted or unsubstituted 4- to 6-membered heterocyclyl containing one oxygen atom; preferably substituted or unsubstituted oxetanyl, substituted or unsubstituted tetrahydrofuranyl, or substituted or unsubstituted tetrahydro-2H-pyranyl; more preferably substituted or unsubstituted oxetan-2-yl, substituted or unsubstituted tetrahydrofuran-3-yl, substituted or unsubstituted tetrahydrofuran-2-yl, substituted or unsubstituted tetrahydro-2H-pyran-4-yl, or substituted or unsubstituted tetrahydro-2H-pyran-3-yl.

In some embodiments, moiety B is 3-dimethylamino-oxetan-2-yl, 4-dimethylamino-tetrahydrofuran-3-yl, 3-dimethylamino-tetrahydrofuran-2-yl, 3-dimethylamino-tetrahydro-2H-pyran-4-yl, or 4-dimethylamino-tetrahydro-2H-pyran-3-yl, wherein said each of 3-dimethylamino-oxetan-2-yl, 4-dimethylamino-tetrahydrofuran-3-yl, 3-dimethylamino-tetrahydrofuran-2-yl, 3-dimethylamino-tetrahydro-2H-pyran-4-yl, and 4-dimethylamino-tetrahydro-2H-pyran-3-yl is optionally substituted with methyl, F, hydroxy, methoxy, or difluoromethyl.

In some embodiments, moiety B is

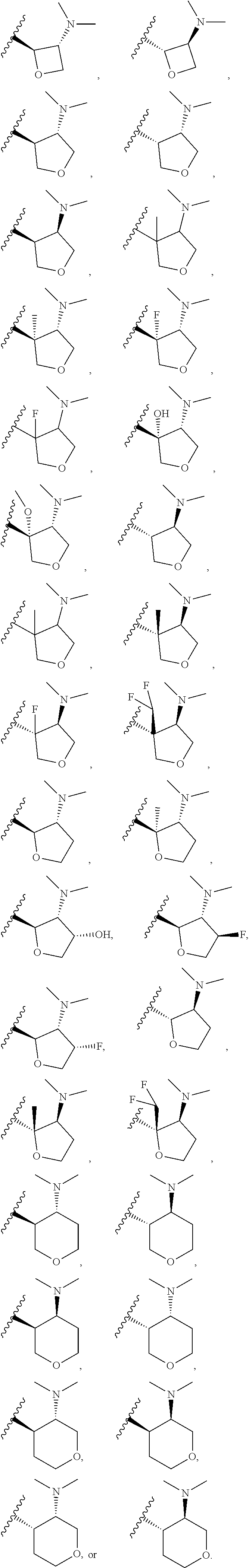

In some embodiments, moiety B is

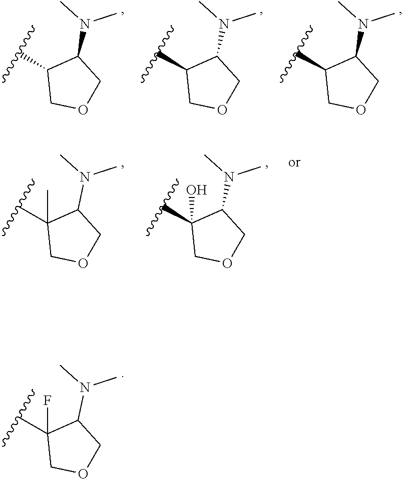

In some embodiments, moiety B is

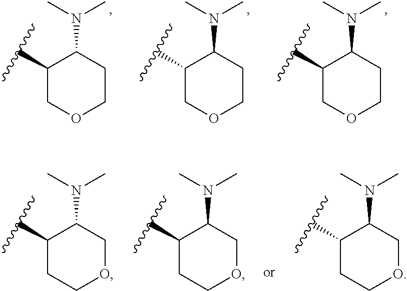

In some embodiments, moiety B is substituted or unsubstituted pyrrolidinyl; preferably substituted or unsubstituted pyrrolidine-2-yl optionally substituted with one or more substituents selected from H, F, methyl, methyl-d3, difluoromethyl, ethyl, 2-fluoroethyl, 2-(methoxy-d3)ethyl, 1-allyl-2-methyl, methoxy, cyclopropylmethyl, oxetanyl, tetrahydrofuranyl, or tetrahydro-2H-pyranyl.

In some embodiments, moiety B is

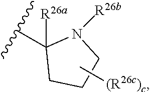

wherein
R^{26a} is H, methyl, or difluoromethyl;
R^{26b} is methyl, methyl-d3, ethyl, 2-fluoroethyl, 2-(methoxy-d3)ethyl, 2-methoxyethyl, 1-allyl-2-methyl, cyclopropylmethyl, oxetanyl, tetrahydrofuranyl, or tetrahydro-2H-pyranyl;
each R^{26c} is, independently, H, F or methoxy, or one pair of R^{26c}, together with the atom(s) to which they are attached form a substituted or unsubstituted cyclopropyl; and
c is 0, 1, or 2

In some embodiments, the carbon atom which R^{26a} connects to is S-configuration.

In some embodiments, the carbon atom which R^{26a} connects to is R-configuration.

In some embodiments, moiety B is

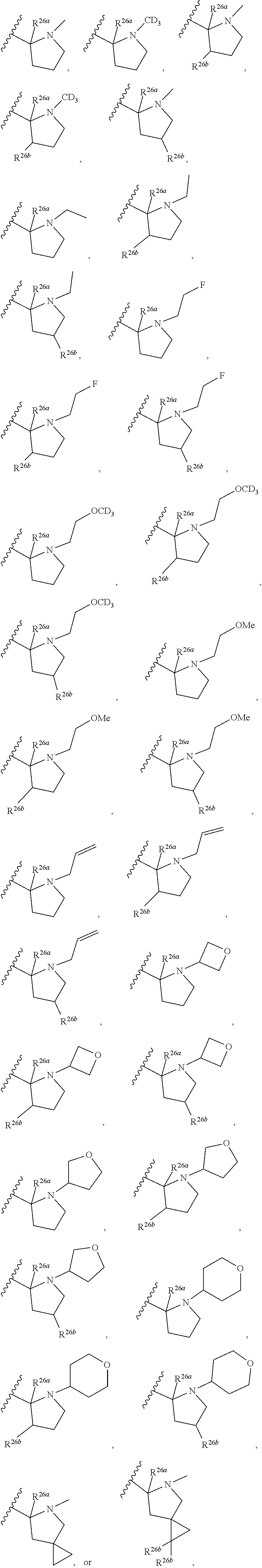

wherein R^{26a} is H, methyl, or difluoromethyl; each R^{26b} is, independently, methoxy, methoxy-d3, fluoro, methyl, fluoromethyl, or difluoromethyl.

In some embodiments, moiety B is

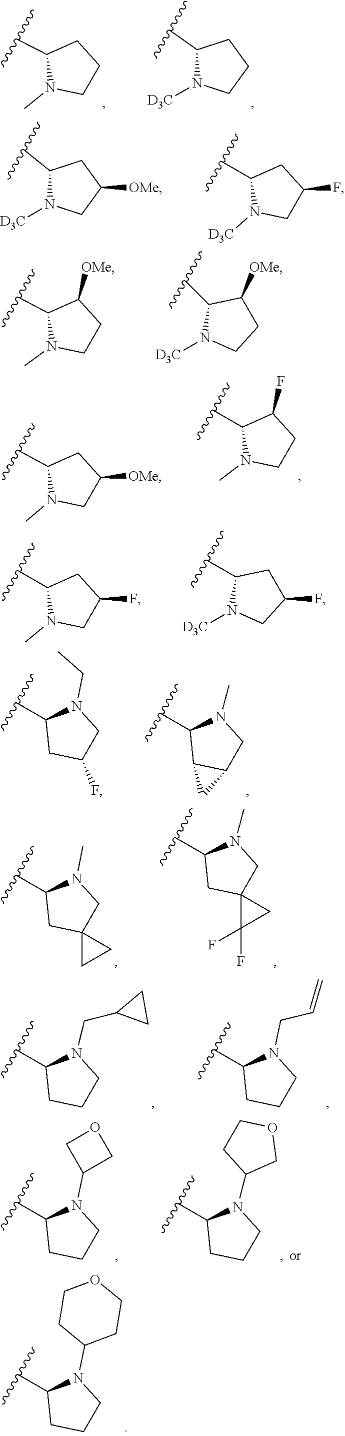

In some embodiments, moiety B is

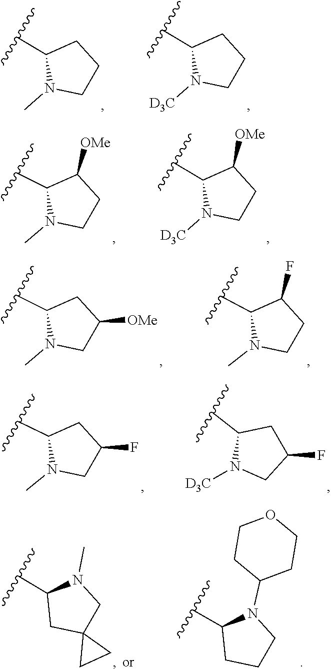

In some embodiments, moiety B is

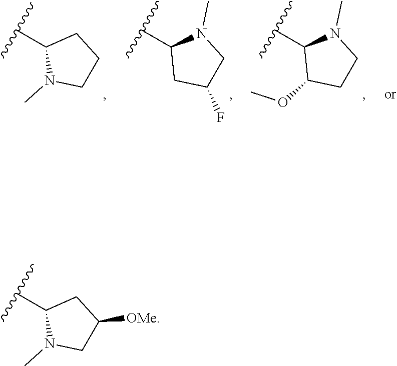

In some embodiments, moiety B is substituted or unsubstituted 6-membered heterocyclyl containing one or two heteroatoms selected from O or N, provided at least one heteroatom is N; preferably moiety B is substituted or unsubstituted piperidinyl, substituted or unsubstituted morpholinyl, substituted or unsubstituted piperazinyl.

In some embodiments, moiety B is

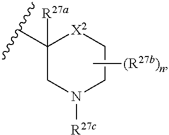

wherein
   X² is —CHF—, —CF₂—, —CH(CH₃)—, N(CH₃)—, —CH(OCH₃)—, —CH(CHF₂)—, —C(═CHF)—, or —O—;
   R^{27a} is methyl, ethyl, difluoromethyl, or trifluoromethyl;
   R^{27b} is H, F, methyl, or methoxy;
   R^{27c} is C₁₋₃alkyl, oxetanyl, tetrahydrofuranyl, or tetrahydropyranyl, wherein said C₁₋₃alkyl is optionally substituted with one or more substituents selected from methyl, methyl-d3, F or alkenyl; and
   n is 0, 1, 2, 3, or 4.

In one embodiment, n is 0. In one embodiment, n is 1. In one embodiment, n is 2. In one embodiment, n is 3. In one embodiment, n is 4.

In some embodiments, the carbon atom of cyclopropyl attached to R^{27a} is R-configuration.

In some embodiments, the carbon atom of cyclopropyl attached to R^{27a} is S-configuration.

In some embodiments, moiety B is

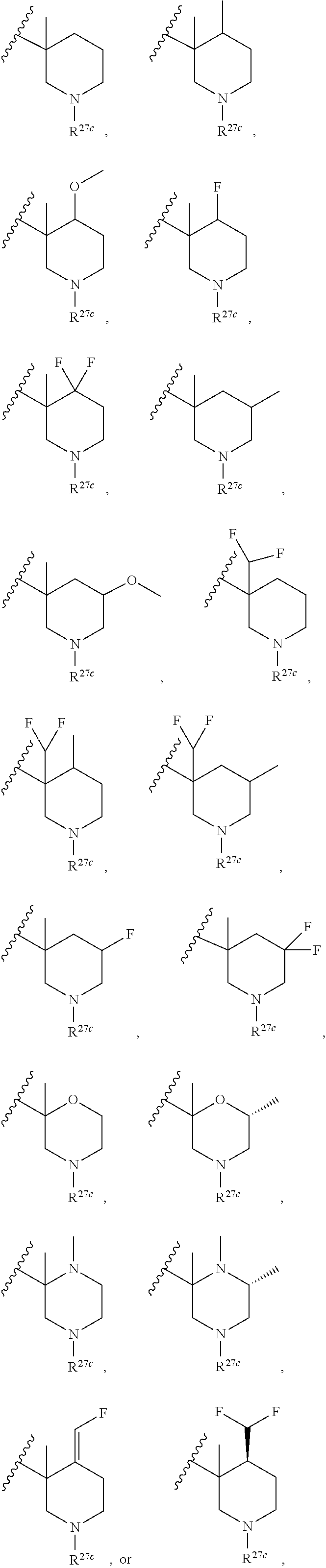

wherein R^{27c} is C₁₋₃alkyl, oxetanyl, tetrahydrofuranyl, or tetrahydropyranyl, wherein said C₁₋₃alkyl is optionally substituted with one or more substituents selected from deuterium, methyl, methyl-d3, F, cyclopropylmethyl, or alkenyl. In some embodiments, moiety B is

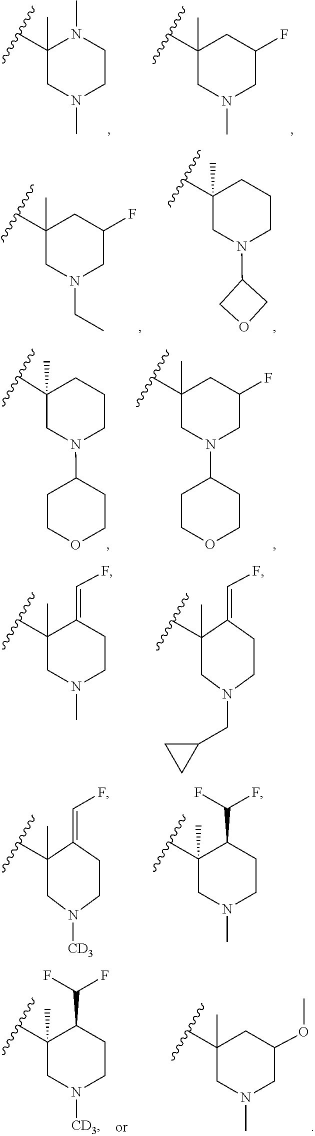

In some embodiments, moiety B is

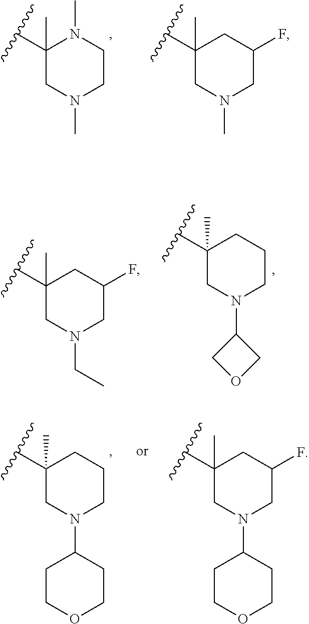

In some embodiments, moiety B is

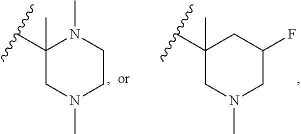

In some embodiments, moiety B is substituted or unsubstituted bicyclic heterocyclyl.

In some embodiments, moiety B is substituted or unsubstituted bicyclic heterocyclyl containing one or two heteroatoms selected from N or O, provided at least one of fusion atoms is N; preferably substituted or unsubstituted hexahydro-1H-pyrrolizinyl, substituted or unsubstituted octahydroindolizinyl, substituted or unsubstituted hexahydro-1H-pyrrolo[2,1-c][1,4]oxazinyl, or substituted or unsubstituted octahydropyrido[2,1-c][1,4]oxazinyl.

In some embodiments, moiety B is

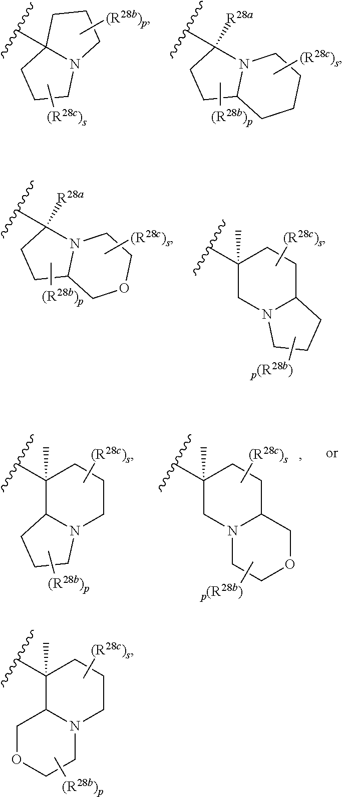

wherein
R^{28a} is H, methyl, methyl-d3, fluoromethyl, difluoromethyl, or trifluoromethyl;
each of R^{28b} and R^{28c} is H, halogen, substituted or unsubstituted alkyl, substituted or unsubstituted alkoxy, or one pair of R^{28b} or R^{28c} together with the atom(s) to which they are attached to, form unsubstituted or substituted alkenyl, or unsubstituted or substituted cycloalkyl; and
each of s and p is, independently, 0, 1, 2, 3, or 4.

In one embodiment, s is 0. In one embodiment, s is 1. In one embodiment, s is 2. In one embodiment, s is 3. In one embodiment, s is 4. In one embodiment, p is 0. In one embodiment, p is 1. In one embodiment, p is 2. In one embodiment, p is 3. In one embodiment, p is 4.

In some embodiments, moiety B is

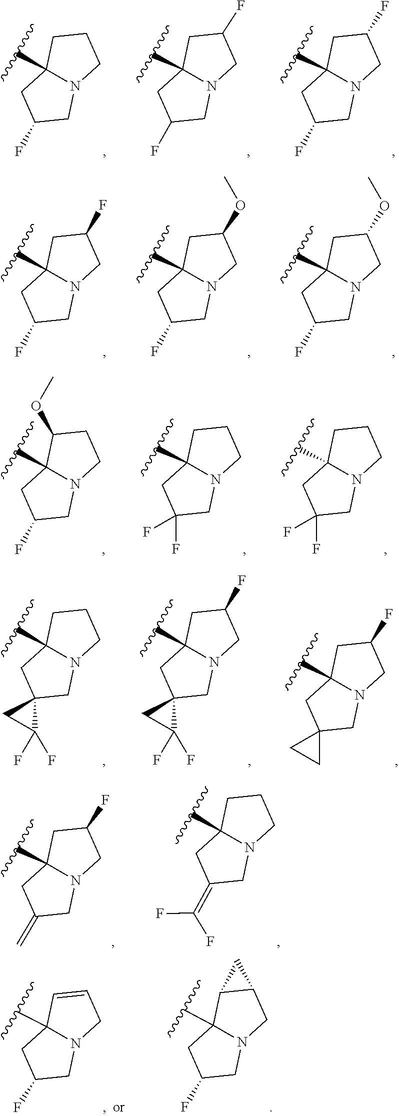

In some embodiments, moiety B is

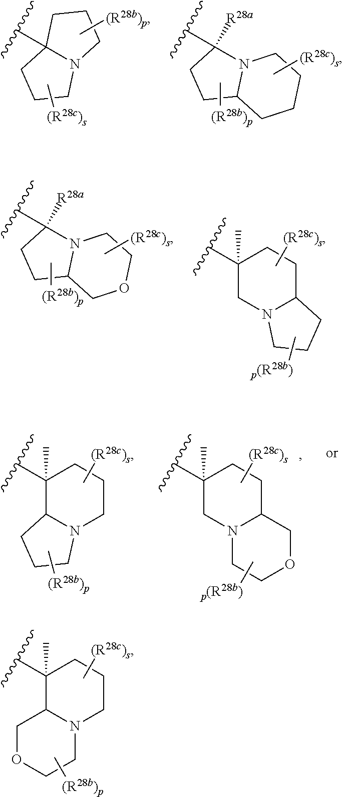

wherein
R^{28a} is H, methyl, methyl-d3, fluoromethyl, difluoromethyl, or trifluoromethyl;
each of R^{28b} and R^{28c} is H, halogen, substituted or unsubstituted alkyl, substituted or unsubstituted alkoxy, or one pair of R^{28b} or R^{28c} together with the atom(s) to which they are attached to, form unsubstituted or substituted alkenyl, or unsubstituted or substituted cycloalkyl; and
each of s and p is, independently, 0, 1, 2, 3, or 4;
provided

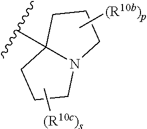

is not

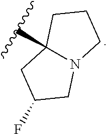 [SMILES: *[C@@]12CCCN1C[C@H](F)C2]

In some embodiments, moiety B is

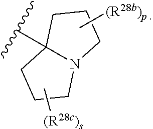

In one embodiment, s is 0. In one embodiment, s is 1. In one embodiment, s is 2. In one embodiment, s is 3. In one embodiment, s is 4. In one embodiment, p is 0. In one embodiment, p is 1. In one embodiment, p is 2. In one embodiment, p is 3. In one embodiment, p is 4.

In some embodiments, one pair of R^{28b} together with the atom(s) to which they are attached to, form ethenyl. In some embodiments, one pair of R^{28c} together with the atom(s) to which they are attached to, form ethenyl. In some embodiments, one pair of R^{28c} together with the atom to which they are attached to, form 2,2-difluoroethenyl. In some embodiments, one pair of R^{28b} together with the atoms to which they are attached to, form cyclopropyl. In some embodiments, one pair of R^{28c} together with the atom to which they are attached to, form cyclopropyl that is optionally substituted with one or more fluoro.

In one embodiment, one pair of R^{28b} together with the atoms to which they are attached to, form unsubstituted or substituted alkenyl, wherein the alkenyl is in the ring to which R^{28b} is attached.

In one embodiment, one pair of R^{28b} or one pair of R^{28c} together with the atom to which they are attached to, form ethenyl which is optionally substituted with one or more F.

In one embodiment, one pair of R^{28b} together with the atoms to which they are attached to, form cyclopropyl.

In one embodiment, one pair of R^{28c} together with the atom to which they are attached to, form cyclopropyl.

In one embodiment, each of R^{28b} and R^{28c} is F, or methoxy.

In some embodiments, moiety B is

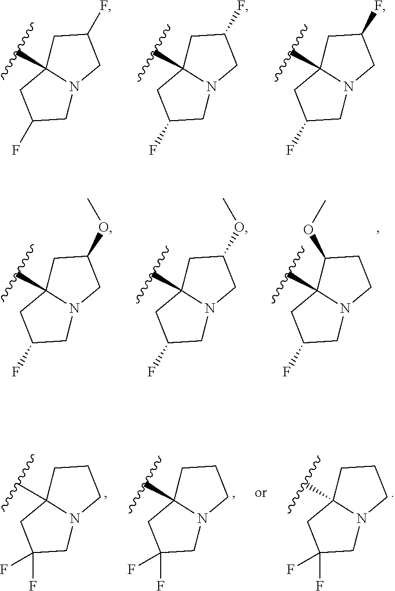

In some embodiments, moiety B is

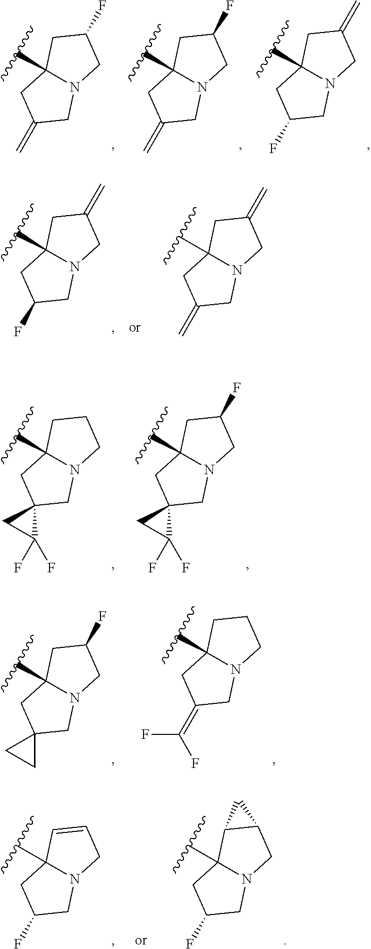

In some embodiments, moiety B is

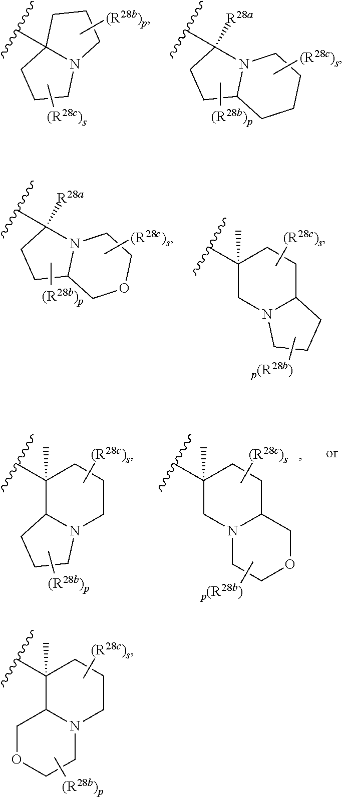

wherein
R^{28a} is H, methyl, methyl-d3, fluoromethyl, difluoromethyl, or trifluoromethyl;
each of R^{28b} and R^{28c} is H, halogen, substituted or unsubstituted alkyl, substituted or unsubstituted alkoxy, or one pair of R^{28b} or R^{28c} together with the atom(s) to which they are attached to, form unsubstituted or substituted alkenyl, or unsubstituted or substituted cycloalkyl; and
each of s and p is, independently, 0, 1, 2, 3, or 4;
provided

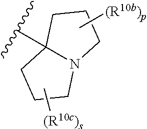

is not

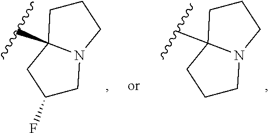 [SMILES: *[C@@]12CCCN1C[C@H](F)C2 ; *C12CCCN1CCC2]

In one embodiment, s is 0. In one embodiment, s is 1. In one embodiment, s is 2. In one embodiment, s is 3. In one embodiment, s is 4. In one embodiment, p is 0. In one embodiment, p is 1. In one embodiment, p is 2. In one embodiment, p is 3. In one embodiment, p is 4.

In some embodiments, moiety B is substituted or unsubstituted bicyclic heterocyclyl containing one or two heteroatoms selected from N or O, provided both fusion atoms are C; preferably substituted or unsubstituted octahydro-1H-cyclopenta[b]pyridinyl, substituted or unsubstituted octahydrocyclopenta[b][1,4]oxazinyl, or substituted or unsubstituted octahydrofuro[3,2-b]pyridinyl.

In some embodiments, moiety B is

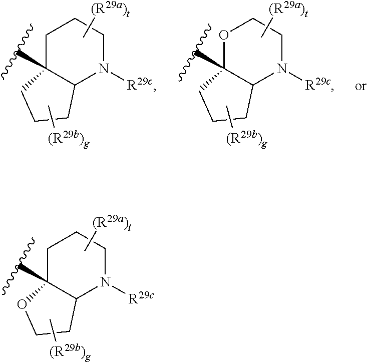

wherein
R^{29c} is methyl, methyl-d3, difluoromethyl, or trifluoromethyl;
each of R^{29a} and R^{29b} is H, halogen, substituted or unsubstituted alkyl; and
each of t and g is, independently, 0, 1, 2, 3, or 4.

In one embodiment, t is 0. In one embodiment, t is 1. In one embodiment, t is 2. In one embodiment, t is 3. In one embodiment, t is 4. In one embodiment, g is 0. In one embodiment, g is 1. In one embodiment, g is 2. In one embodiment, g is 3. In one embodiment, g is 4.

In some embodiments, moiety B is substituted or unsubstituted cyclobutyl, substituted or unsubstituted cyclopentyl, substituted or unsubstituted cycloheptyl; preferably substituted or unsubstituted 2-dimethylamino-cyclobutyl, substituted or unsubstituted 2-dimethylamino-cyclopentyl, substituted or unsubstituted 2-dimethylamino-cycloheptyl; more preferably 2-dimethylamino-cyclobutyl, 2-dimethylamino-cyclopentyl, or 2-dimethylamino-cycloheptyl, wherein said each of 2-dimethylamino-cyclobutyl, 2-dimethylamino-cyclopentyl and 2-dimethylamino-cycloheptyl is optionally substituted with H, halogen, methoxy, or methoxy-d3.

In some embodiments, moiety B is

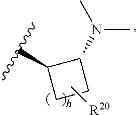

wherein
R²⁰ is H, halogen, methyl, methyl-d3, methoxy, or methoxy-d3; and
h is 1, 2, or 3.

In some embodiments, the compound of formula (IIb) is a compound of formula (IVb):

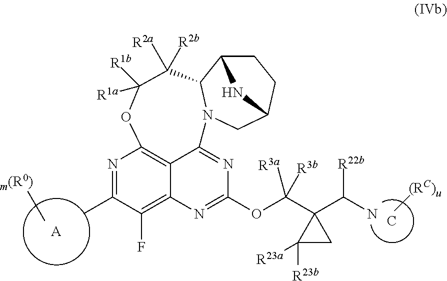

wherein
ring C is is substituted or unsubstituted 4- to 7-membered heterocyclyl optionally containing one or more additional heteroatom selected from N or O;
each of R^{c} is, independently, H, halogen, amino, —OH, —CN, unsubstituted or substituted C₁₋₄alkyl, or unsubstituted or substituted C₁₋₄alkoxy, or one pair of the R^{c} groups, together with the atom(s) to which they are attached to, form unsubstituted or substituted bridge, unsubstituted or substituted cycloalkyl, or unsubstituted or substituted heterocyclyl.
u is an integer from 0 to the maximum number of the substituent groups allowed on rings C; and
the other variables are defined above.

In some embodiments, the compound of formula (IVb) is a compound of formula (Vb):

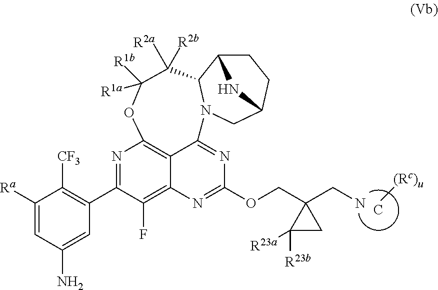

wherein
R^{a} is methyl, methyl-d3, or Cl.
ring C is is substituted or unsubstituted 4- to 7-membered heterocyclyl optionally containing one or more additional heteroatom selected from N, O, or S;
each of R^{c} is, independently, H, halogen, amino, —OH, —CN, unsubstituted or substituted C₁₋₄alkyl, or unsubstituted or substituted C₁₋₄alkoxy, or one pair of the R^{c} groups, together with the atom(s) to which they are attached to, form unsubstituted or substituted bridge, unsubstituted or substituted cycloalkyl, or unsubstituted or substituted heterocyclyl.
u is an integer from 0 to the maximum number of the substituent groups allowed on rings C; and
the other variables are defined above.

In some embodiments, ring C is unsubstituted or substituted azetidinyl, unsubstituted or substituted pyrrolidinyl, unsubstituted or substituted piperidinyl, unsubstituted or substituted morpholinyl, unsubstituted or substituted thiomorpholine, or unsubstituted or substituted 1,4-oxazepan-4-yl.

In some embodiments, each of R^{c} is, independently, H, methyl, methoxy, F, trifluoromethyl, hydroxy, or hydroxymethyl.

In some embodiments, one pair of the R^{c} groups, together with the atoms to which they are attached to, form unsubstituted or substituted bridge, and the bridge is —CH₂—, or —CH₂—CH₂—.

In some embodiments, one pair of the R^{c} groups, together with the atoms to which they are attached to, form unsubstituted or substituted cyclopropyl, unsubstituted or substituted tetrahydrofuranyl, unsubstituted or substituted imidazol-yl, or unsubstituted or substituted 1,2,4-triazol-yl, wherein said tetrahydrofuranyl, imidazol-yl and 1,2,4-triazol-yl are fused to ring C.

In some embodiments, one pair of the R^{c} groups, together with the atom to which they are attached to, form unsubstituted or substituted cyclopropyl, unsubstituted or substituted cyclobutyl, unsubstituted or substituted oxetanyl, unsubstituted or substituted tetrahydrofuranyl, unsubstituted or substituted tetrahydro-2H-pyran-yl.

In some embodiments, each of R^{c} is, independently, F, methyl, trifluoromethyl, hydroxy, methoxy, or hydroxymethyl.

In some embodiments, u is 0. In some embodiments, u is 1. In some embodiments, u is 2. In some embodiments, u is 3.

In some embodiments,

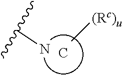

is

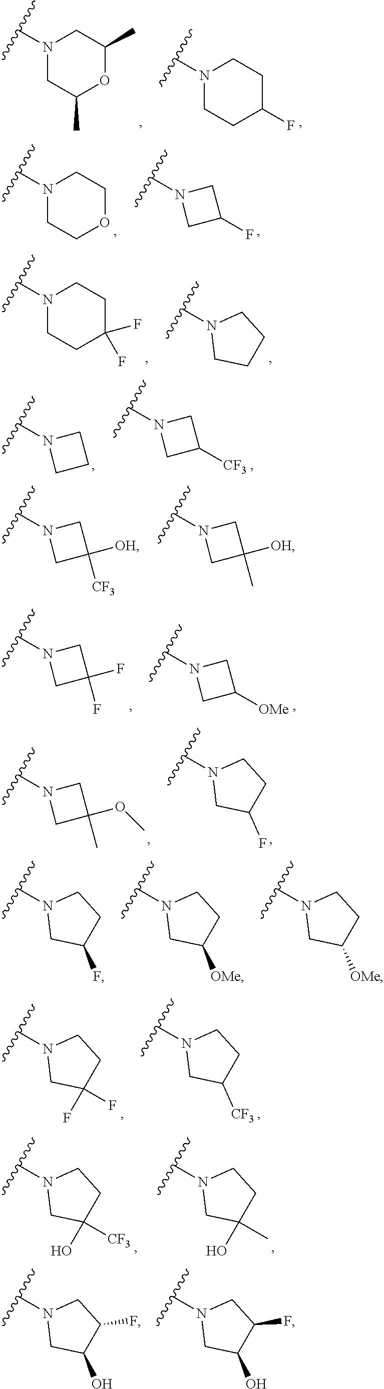

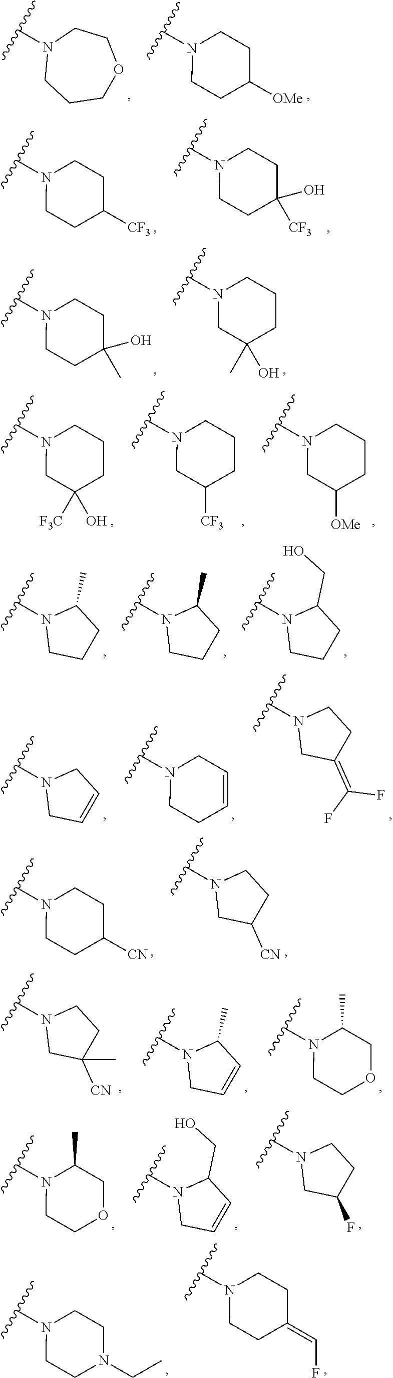

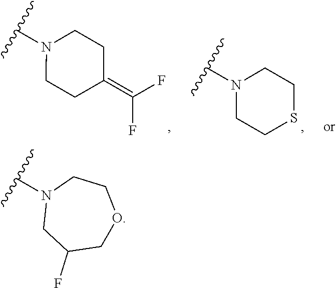 [SMILES: *N1CCC(=C(F)F)CC1 ; *N1CCSCC1 ; *N1CCOCC(F)C1]

In some embodiments,

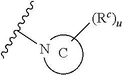

is

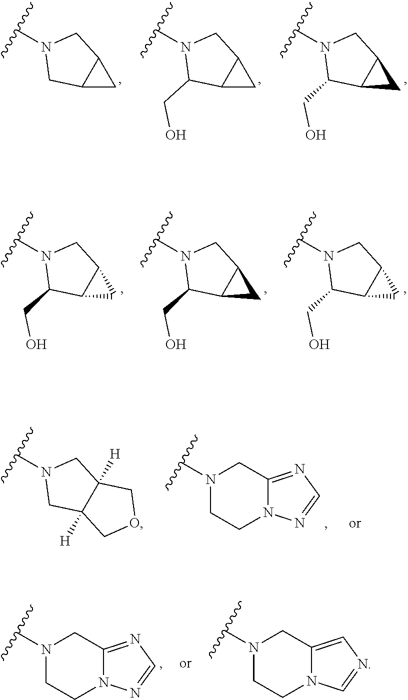

In some embodiments,

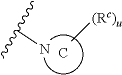

is

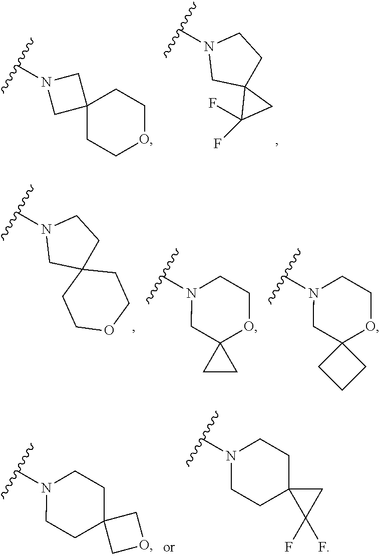

In some embodiments,

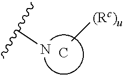

is

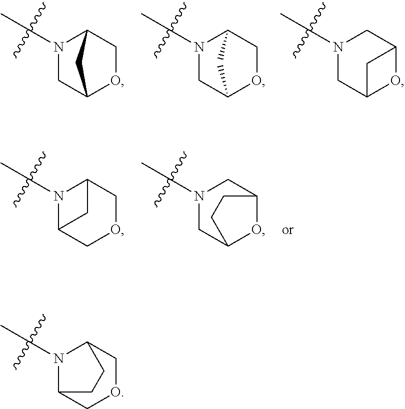

In some embodiments,

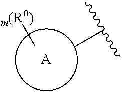

is

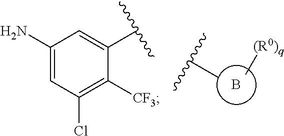

is

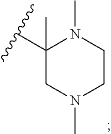 [SMILES: *C1(C)CN(C)CCN1C]

and all of R^{1a}, R^{1b}, R^{2a} and R^{2b} are H.

In some embodiments,

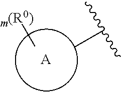

is

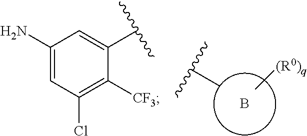

is

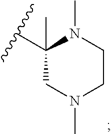 [SMILES: *[C@]1(C)CN(C)CCN1C]

and all of R^{1a}, R^{1b}, R^{2a} and R^{2b} are H.

In some embodiments,

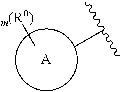

is

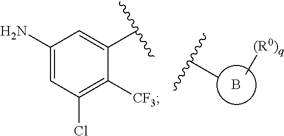

is

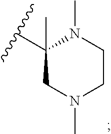 [SMILES: *[C@@]1(C)CN(C)CCN1C]

and all of R^{1a}, R^{1b}, R^{2a} and R^{2b} are H.

In some embodiments,

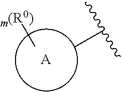

is

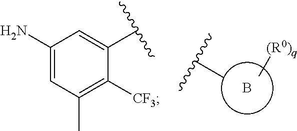

is

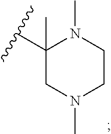 [SMILES: *C1(C)CN(C)CCN1C]

and all of R^{1a}, R^{1b}, R^{2a} and R^{2b} are H.

In some embodiments,

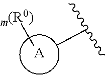

is

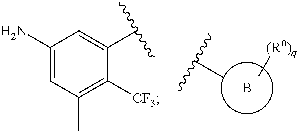

is

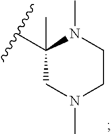 [SMILES: *[C@]1(C)CN(C)CCN1C]

and all of R^{1a}, R^{1b}, R^{2a} and R^{2b} are H.

In some embodiments,

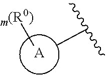

is

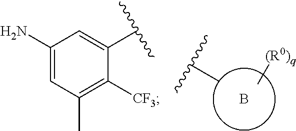

is

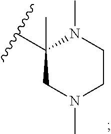 [SMILES: *[C@@]1(C)CN(C)CCN1C]

and all of R^{1a}, R^{1b}, R^{2a} and R^{2b} are H.

In some embodiments,

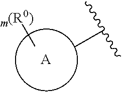

is

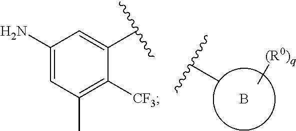

is

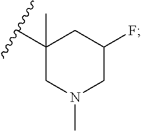 [SMILES: *C1(C)CC(F)CN(C)C1]

and all of R^{1a}, R^{1b}, R^{2a} and R^{2b} are H.

In some embodiments,

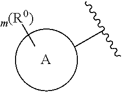

is

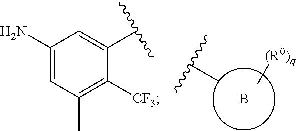

is

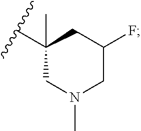

and all of R^{1a}, R^{1b}, R^{2a} and R^{2b} are H.

In some embodiments,

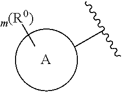

is

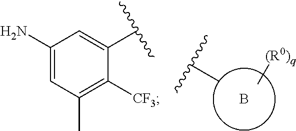

is

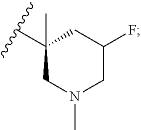

and all of R^{1a}, R^{1b}, R^{2a} and R^{2b} are H.

In some embodiments,

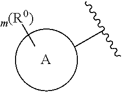

is

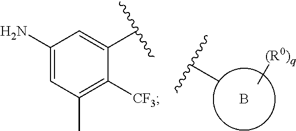

is

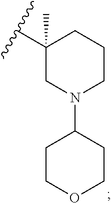 [SMILES: *[C@@]1(C)CCCN(C2CCOCC2)C1]

and all of R^{1a}, R^{1b}, R^{2a} and R^{2b} are H.

In some embodiments,

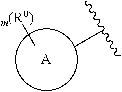

is

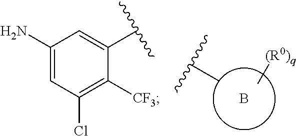

is

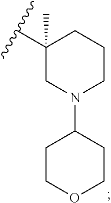 [SMILES: *[C@@]1(C)CCCN(C2CCOCC2)C1]

and all of R^{1a}, R^{1b}, R^{2a} and R^{2b} are H.

In some embodiments,

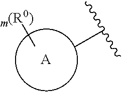

is

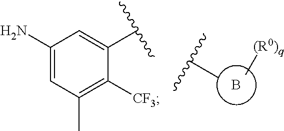

is

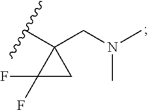 [SMILES: *C1(CN(C)C)CC1(F)F]

and all of R^{1a}, R^{1b}, R^{2a} and R^{2b} are H.

In some embodiments,

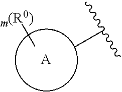

is

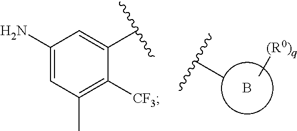

is

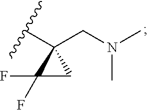 [SMILES: *[C@]1(CN(C)C)CC1(F)F]

and all of R^{1a}, R^{1b}, R^{2a} and R^{2b} are H.

In some embodiments,

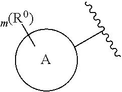

is

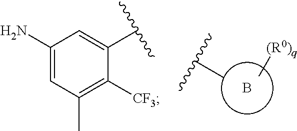

is

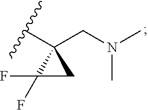 [SMILES: *[C@@]1(CN(C)C)CC1(F)F]

and all of R^{1a}, R^{1b}, R^{2a} and R^{2b} are H.

In some embodiments,

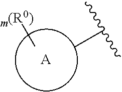

is

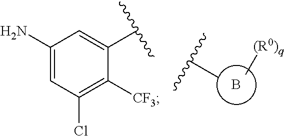

is

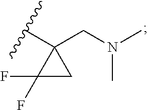 [SMILES: *C1(CN(C)C)CC1(F)F]

and all of R^{1a}, R^{1b}, R^{2a} and R^{2b} are H.

In some embodiments,

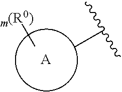

is

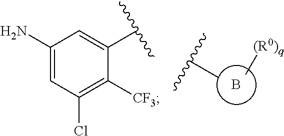

is

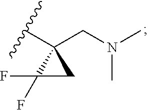 [SMILES: *[C@@]1(CN(C)C)CC1(F)F]

and all of R^{1a}, R^{1b}, R^{2a} and R^{2b} are H.

In some embodiments,

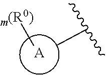

is

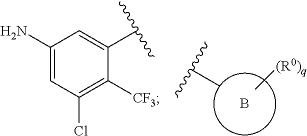

is

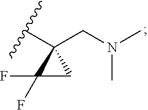 [SMILES: *[C@]1(CN(C)C)CC1(F)F]

and all of R^{1a}, R^{1b}, R^{2a} and R^{2b} are H.

In some embodiments,

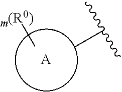

is

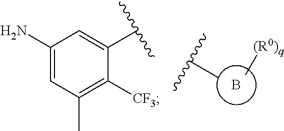

is

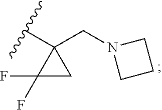 [SMILES: *C1(CN2CCC2)CC1(F)F]

and all of R^{1a}, R^{1b}, R^{2a} and R^{2b} are H.

In some embodiments,

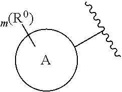

is

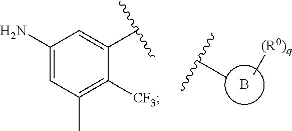

is

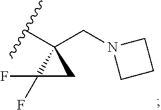 [SMILES: *[C@@]1(CN2CCC2)CC1(F)F]

and all of R^{1a}, R^{1b}, R^{2a} and R^{2b} are H.

In some embodiments,

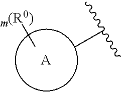

is

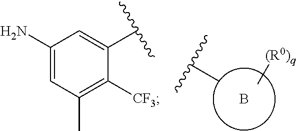

is

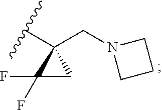 [SMILES: *[C@]1(CN2CCC2)CC1(F)F]

and all of R^{1a}, R^{1b}, R^{2a} and R^{2b} are H.

In some embodiments,

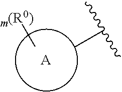

is

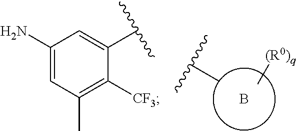

is

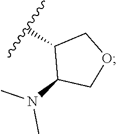 [SMILES: *[C@@H]1COC[C@H]1N(C)C]

and all of R^{1a}, R^{1b}, R^{2a} and R^{2b} are H.

In some embodiments,

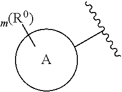

is

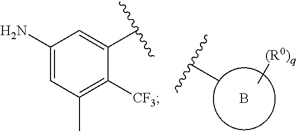

is

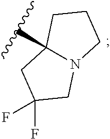 [SMILES: *[C@@]12CCCN1CC(F)(F)C2]

and all of R^{1a}, R^{1b}, R^{2a} and R^{2b} are H.

In some embodiments,

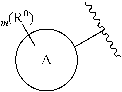

is

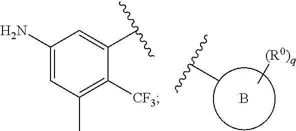

is

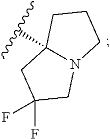 [SMILES: *[C@]12CCCN1CC(F)(F)C2]

and all of R^{1a}, R^{1b}, R^{2a} and R^{2b} are H.

In some embodiments,

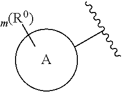

is

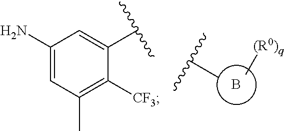

is

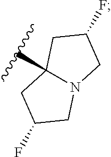 [SMILES: *[C@@]12C[C@H](F)CN1C[C@H](F)C2]

and all of R^{1a}, R^{1b}, R^{2a} and R^{2b} are H.

In some embodiments,

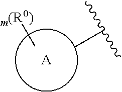

is

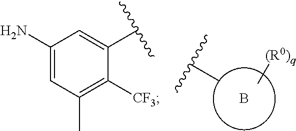

is

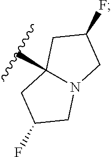

and all of R^{1a}, R^{1b}, R^{2a} and R^{2b} are H.

In some embodiments,

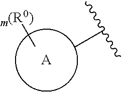

is

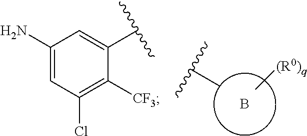

is

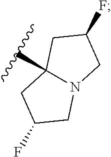

and all of R^{1a}, R^{1b}, R^{2a} and R^{2b} are H.

In some embodiments,

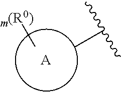

is

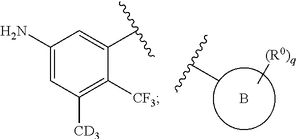

is

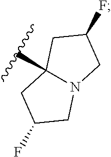

and all of R^{1a}, R^{1b}, R^{2a} and R^{2b} are H.

In some embodiments,

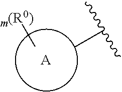

is

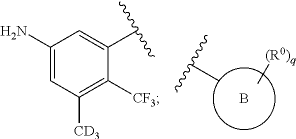

is

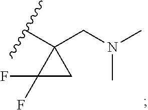 [SMILES: *C1(CN(C)C)CC1(F)F]

and all of R^{1a}, R^{1b}, R^{2a} and R^{2b} are H.

In some embodiments,

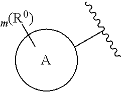

is

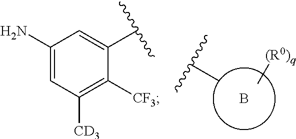

is

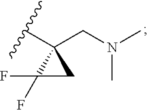 [SMILES: *[C@@]1(CN(C)C)CC1(F)F]

and all of R^{1a}, R^{1b}, R^{2a} and R^{2b} are H.

In some embodiments,

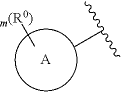

is

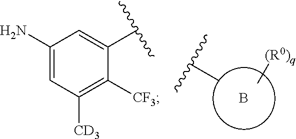

is

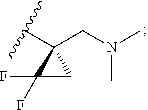 [SMILES: *[C@]1(CN(C)C)CC1(F)F]

and all of R^{1a}, R^{1b}, R^{2a} and R^{2b} are H.

In some embodiments,

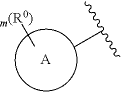

is

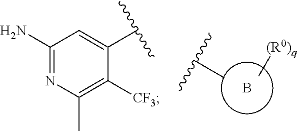

is

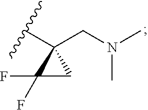 [SMILES: *[C@]1(CN(C)C)CC1(F)F]

and all of R^{1a}, R^{1b}, R^{2a} and R^{2b} are H.

In some embodiments,

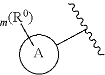

is

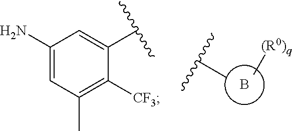

is

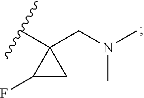 [SMILES: *C1(CN(C)C)CC1F]

and all of R^{1a}, R^{1b}, R^{2a} and R^{2b} are H.

In some embodiments,

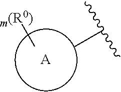

is

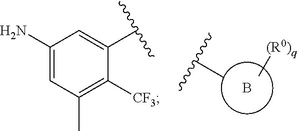

is

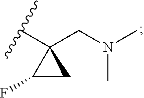 [SMILES: *[C@@]1(CN(C)C)C[C@@H]1F]

and all of R^{1a}, R^{1b}, R^{2a} and R^{2b} are H.

In some embodiments,

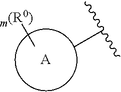

is

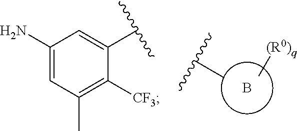

is

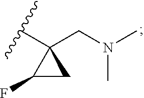 [SMILES: *[C@@]1(CN(C)C)C[C@H]1F]

and all of R^{1a}, R^{1b}, R^{2a} and R^{2b} are H.

In some embodiments,

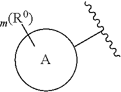

is

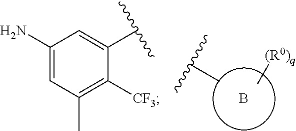

is

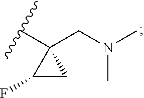 [SMILES: *[C@]1(CN(C)C)C[C@@H]1F]

and all of R^{1a}, R^{1b}, R^{2a} and R^{2b} are H.

In some embodiments,

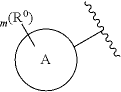

is

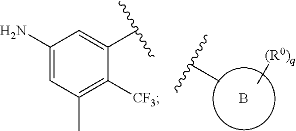

is

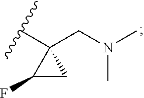 [SMILES: *[C@]1(CN(C)C)C[C@H]1F]

and all of R^{1a}, R^{1b}, R^{2a} and R^{2b} are H.

In some embodiments,

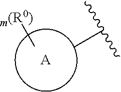

### is C

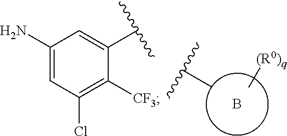

is

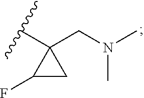 [SMILES: *C1(CN(C)C)CC1F]

and all of R^{1a}, R^{1b}, R^{2a} and R^{2b} are H.

In some embodiments,

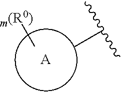

is

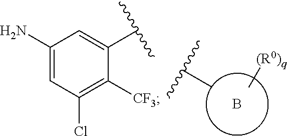

is

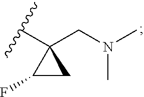 [SMILES: *[C@@]1(CN(C)C)C[C@@H]1F]

and all of R^{1a}, R^{1b}, R^{2a} and R^{2b} are H.

In some embodiments,

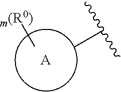

is

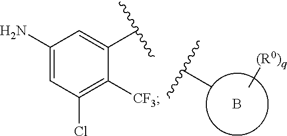

is

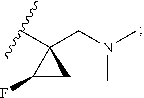 [SMILES: *[C@@]1(CN(C)C)C[C@H]1F]

and all of R^{1a}, R^{1b}, R^{2a} and R^{2b} are H.

In some embodiments,

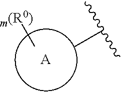

is

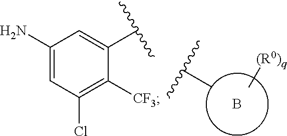

is

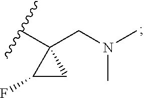 [SMILES: *[C@]1(CN(C)C)C[C@@H]1F]

and all of R^{1a}, R^{1b}, R^{2a} and R^{2b} are H.

In some embodiments,

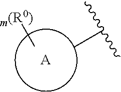

is

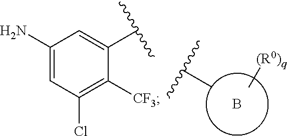

is

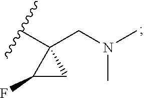 [SMILES: *[C@]1(CN(C)C)C[C@H]1F]

and all of R^{1a}, R^{1b}, R^{2a} and R^{2b} are H.

In some embodiments,

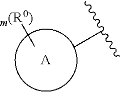

is

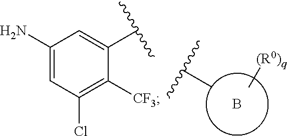

is

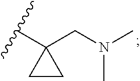 [SMILES: *C1(CN(C)C)CC1]

and all of R^{1a}, R^{1b}, R^{2a} and R^{2b} are H.

In some embodiments,

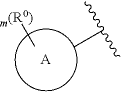

is

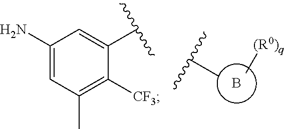

is

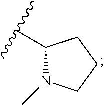 [SMILES: *[C@@H]1CCCN1C]

and all of R^{1a}, R^{1b}, R^{2a} and R^{2b} are H.

In some embodiments,

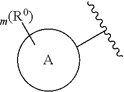

is

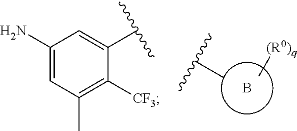

is

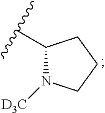 [SMILES: *[C@@H]1CCCN1C([2H])([2H])[2H]]

and all of R^{1a}, R^{1b}, R^{2a} and R^{2b} are H.

In some embodiments,

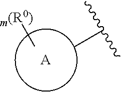

is

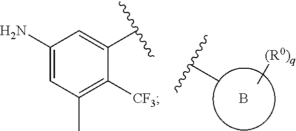

is

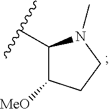 [SMILES: *[C@@H]1[C@@H](OC)CCN1C]

and all of R^{1a}, R^{1b}, R^{2a} and R^{2b} are H.

In some embodiments,

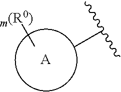

is

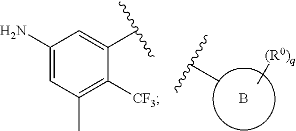

is

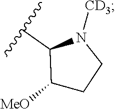 [SMILES: *[C@@H]1[C@@H](OC)CCN1C([2H])([2H])[2H]]

and all of R^{1a}, R^{1b}, R^{2a} and R^{2b} are H.

In some embodiments,

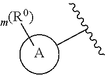

is

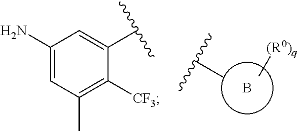

is

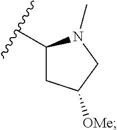 [SMILES: *[C@@H]1C[C@@H](OC)CN1C]

and all of R^{1a}, R^{1b}, R^{2a} and R^{2b} are H.

In some embodiments,

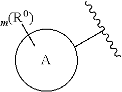

is

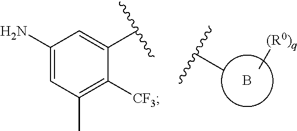

is

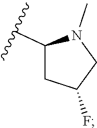 [SMILES: *[C@@H]1C[C@@H](F)CN1C]

and all of R^{1a}, R^{1b}, R^{2a} and R^{2b} are H.

In some embodiments,

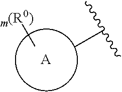

is

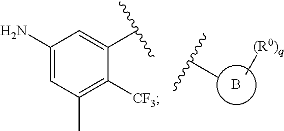

is

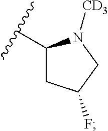 [SMILES: *[C@@H]1C[C@@H](F)CN1C([2H])([2H])[2H]]

and all of R^{1a}, R^{1b}, R^{2a} and R^{2b} are H.

In some embodiments,

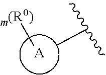

is

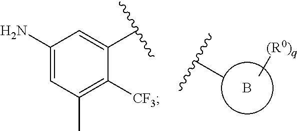

is

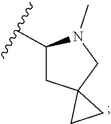 [SMILES: *[C@@H]1CC2(CC2)CN1C]

and all of R^{1a}, R^{1b}, R^{2a} and R^{2b} are H.

In some embodiments,

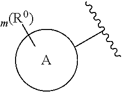

is

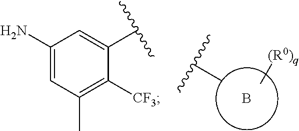

is

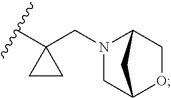 [SMILES: *C1(CN2C[C@@H]3C[C@H]2CO3)CC1]

and all of R^{1a}, R^{1b}, R^{2a} and R^{2b} are H.

In some embodiments,

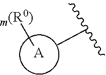

is

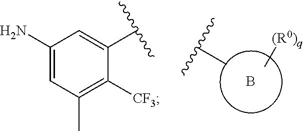

is

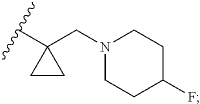 [SMILES: *C1(CN2CCC(F)CC2)CC1]

and all of R^{1a}, R^{1b}, R^{2a} and R^{2b} are H.

In some embodiments,

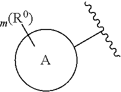

is

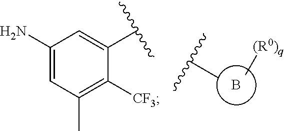

is

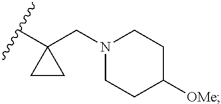 [SMILES: *C1(CN2CCC(OC)CC2)CC1]

and all of R^{1a}, R^{1b}, R^{2a} and R^{2b} are H.

In some embodiments,

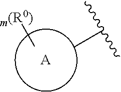

is

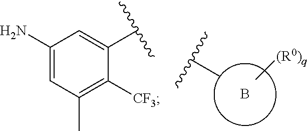

is

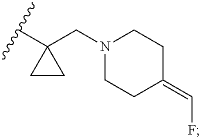 [SMILES: *C1(CN2CCC(=CF)CC2)CC1]

and all of R^{1a}, R^{1b}, R^{2a} and R^{2b} are H.

In some embodiments,

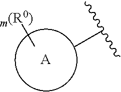

is

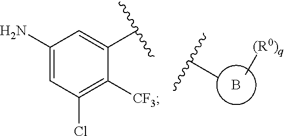

is

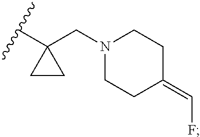 [SMILES: *C1(CN2CCC(=CF)CC2)CC1]

and all of R^{1a}, R^{1b}, R^{2a} and R^{2b} are H.

In some embodiments,

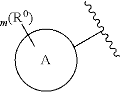

is

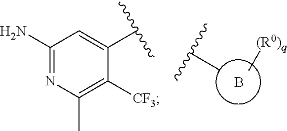

is

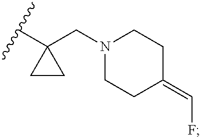 [SMILES: *C1(CN2CCC(=CF)CC2)CC1]

and all of R^{1a}, R^{1b}, R^{2a} and R^{2b} are H.

In some embodiments,

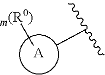

is

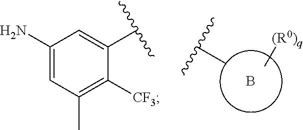

is

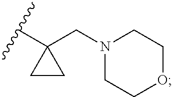 [SMILES: *C1(CN2CCOCC2)CC1]

and all of R^{1a}, R^{1b}, R^{2a} and R^{2b} are H.

In some embodiments,

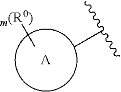

is

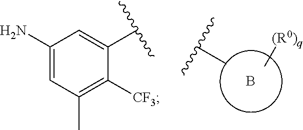

is

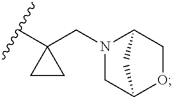 [SMILES: *C1(CN2C[C@H]3C[C@@H]2CO3)CC1]

and all of R^{1a}, R^{1b}, R^{2a} and R^{2b} are H.

In some embodiments,

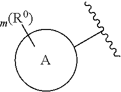

is

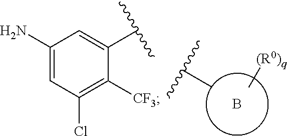

is

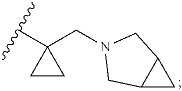 [SMILES: *C1(CN2CC3CC3C2)CC1]

and all of R^{1a}, R^{1b}, R^{2a} and R^{2b} are H.

In some embodiments,

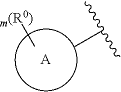

is

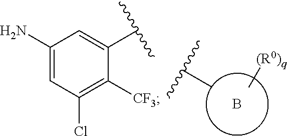

is

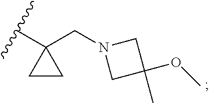 [SMILES: *C1(CN2CC(C)(OC)C2)CC1]

and all of R^{1a}, R^{1b}, R^{2a} and R^{2b} are H.

In some embodiments,

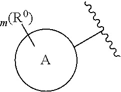

is

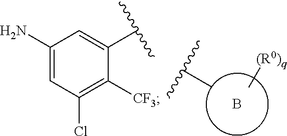

is

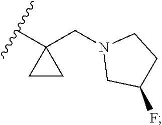 [SMILES: *C1(CN2CC[C@@H](F)C2)CC1]

and all of R^{1a}, R^{1b}, R^{2a} and R^{2b} are H.

In some embodiments,

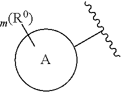

is

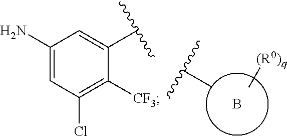

is

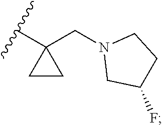 [SMILES: *C1(CN2CC[C@H](F)C2)CC1]

and all of R^{1a}, R^{1b}, R^{2a} and R^{2b} are H.

In some embodiments,

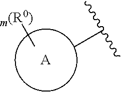

is

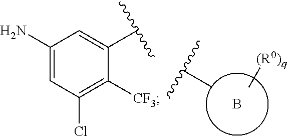

is

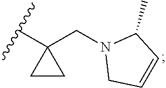 [SMILES: *C1(CN2CC=C[C@H]2C)CC1]

and all of R^{1a}, R^{1b}, R^{2a} and R^{2b} are H.

In some embodiments,

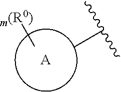

is

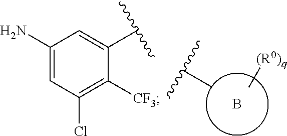

is

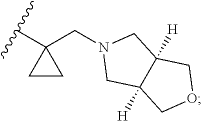 [SMILES: *C1(CN2C[C@H]3COC[C@H]3C2)CC1]

and all of R^{1a}, R^{1b}, R^{2a} and R^{2b} are H.

In some embodiments,

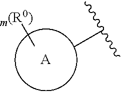

is

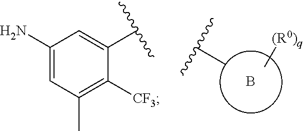

is

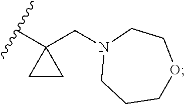 [SMILES: *C1(CN2CCCOCC2)CC1]

and all of R^{1a}, R^{1b}, R^{2a} and R^{2b} are H.

In some embodiments,

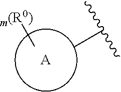

is

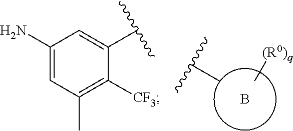

is

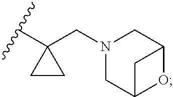 [SMILES: *C1(CN2CC3CC(C2)O3)CC1]

and all of R^{1a}, R^{1b}, R^{2a} and R^{2b} are H.

In some embodiments,

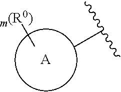

is

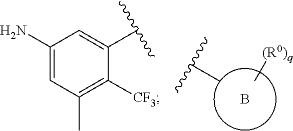

is

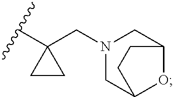 [SMILES: *C1(CN2CC3CCC(C2)O3)CC1]

and all of R^{1a}, R^{1b}, R^{2a} and R^{2b} are H.

In some embodiments,

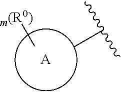

is

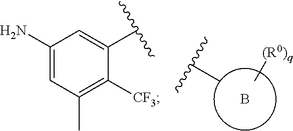

is

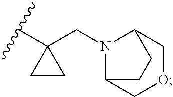 [SMILES: *C1(CN2C3CCC2COC3)CC1]

and all of R^{1a}, R^{1b}, R^{2a} and R^{2b} are H.

In some embodiments,

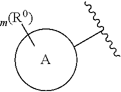

is

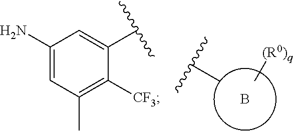

is

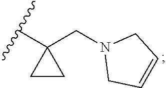 [SMILES: *C1(CN2CC=CC2)CC1]

and all of R^{1a}, R^{1b}, R^{2a} and R^{2b} are H.

In some embodiments,

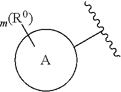

is

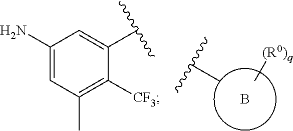

is

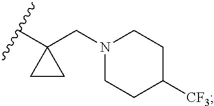 [SMILES: *C1(CN2CCC(C(F)(F)F)CC2)CC1]

and all of R^{1a}, R^{1b}, R^{2a} and R^{2b} are H.

In some embodiments,

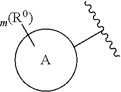

is

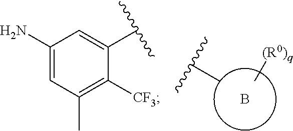

is

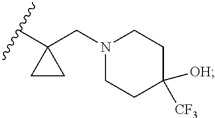 [SMILES: *C1(CN2CCC(O)(C(F)(F)F)CC2)CC1]

and all of R^{1a}, R^{1b}, R^{2a} and R^{2b} are H.

In some embodiments,

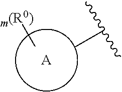

is

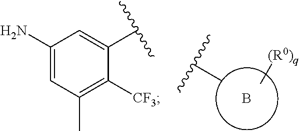

is

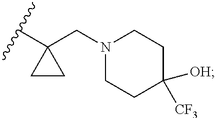 [SMILES: *C1(CN2CCC(O)(C(F)(F)F)CC2)CC1]

and all of R^{1a}, R^{1b}, R^{2a} and R^{2b} are H.

In some embodiments,

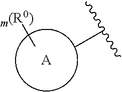

is

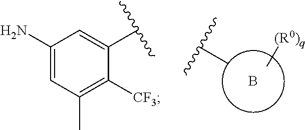

is

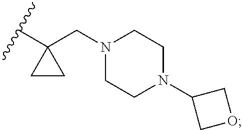 [SMILES: *C1(CN2CCN(C3COC3)CC2)CC1]

and all of R^{1a}, R^{1b}, R^{2a} and R^{2b} are H.

In some embodiments,

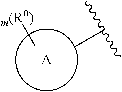

is

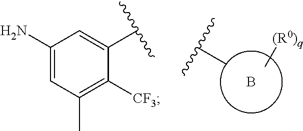

is

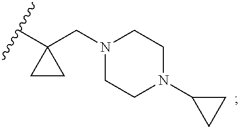 [SMILES: *C1(CN2CCN(C3CC3)CC2)CC1]

and all of R^{1a}, R^{1b}, R^{2a} and R^{2b} are H.

In some embodiments,

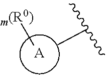

is

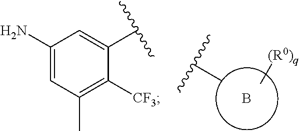

is

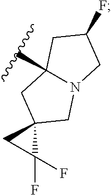 [SMILES: *[C@@]12C[C@@H](F)CN1C[C@@]1(CC1(F)F)C2]

and all of R^{1a}, R^{1b}, R^{2a} and R^{2b} are H.

In some embodiments,

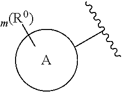

is

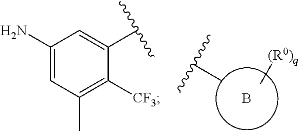

is

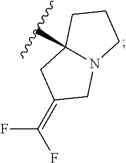 [SMILES: *[C@@]12CCCN1CC(=C(F)F)C2]

and all of R^{1a}, R^{1b}, R^{2a} and R^{2b} are H.

In some embodiments,

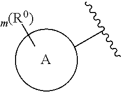

is

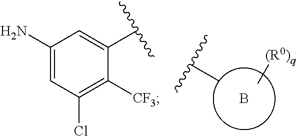

is

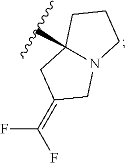 [SMILES: *[C@@]12CCCN1CC(=C(F)F)C2]

and all of R^{1a}, R^{1b}, R^{2a} and R^{2b} are H.

In some embodiments,

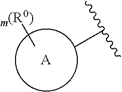

is

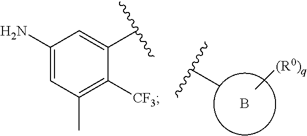

is

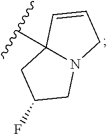 [SMILES: *C12C=CCN1C[C@H](F)C2]

and all of R^{1a}, R^{1b}, R^{2a} and R^{2b} are H.

In some embodiments,

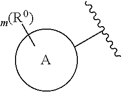

is

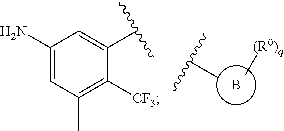

is

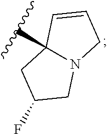 [SMILES: *[C@@]12C=CCN1C[C@H](F)C2]

and all of R^{1a}, R^{1b}, R^{2a} and R^{2b} are H.

In some embodiments,

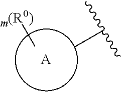

is

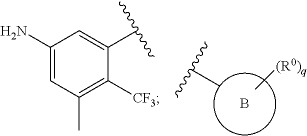

is

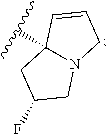 [SMILES: *[C@]12C=CCN1C[C@H](F)C2]

and all of R^{1a}, R^{1b}, R^{2a} and R^{2b} are H.

In some embodiments,

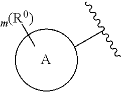

is

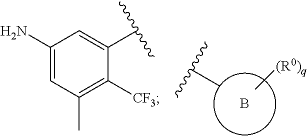

is

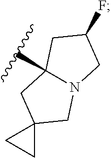 [SMILES: *[C@@]12C[C@@H](F)CN1CC1(CC1)C2]

and all of R^{1a}, R^{1b}, R^{2a} and R^{2b} are H.

In some embodiments,

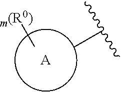

is

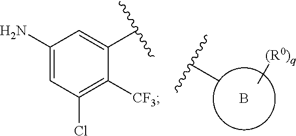

is

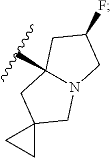 [SMILES: *[C@@]12C[C@@H](F)CN1CC1(CC1)C2]

and all of R^{1a}, R^{1b}, R^{2a} and R^{2b} are H.

In some embodiments,

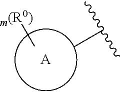

is

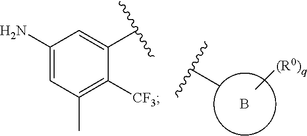

is

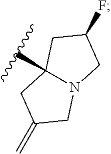 [SMILES: *[C@]12CC(=C)CN1C[C@H](F)C2]

and all of R^{1a}, R^{1b}, R^{2a} and R^{2b} are H.

In some embodiments,

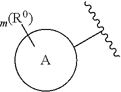

is

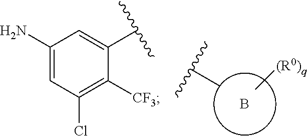

is

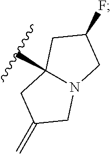 [SMILES: *[C@]12CC(=C)CN1C[C@H](F)C2]

and all of R^{1a}, R^{1b}, R^{2a} and R^{2b} are H.

In some embodiments,

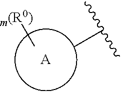

is

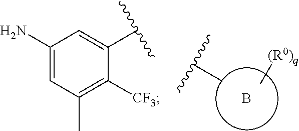

is

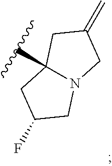 [SMILES: *[C@@]12CC(=C)CN1C[C@H](F)C2]

and all of R^{1a}, R^{1b}, R^{2a} and R^{2b} are H.

In some embodiments,

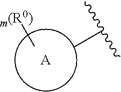

is

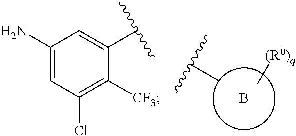

is

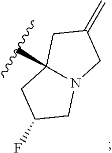 [SMILES: *[C@@]12CC(=C)CN1C[C@H](F)C2]

and all of R^{1a}, R^{1b}, R^{2a} and R^{2b} are H.

In some embodiments,

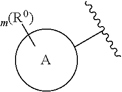

is

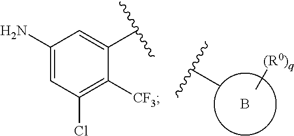

is

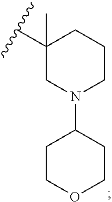 [SMILES: *C1(C)CCCN(C2CCOCC2)C1]

and all of R^{1a}, R^{1b}, R^{2a} and R^{2b} are H.

In some embodiments,

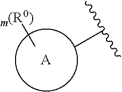

is

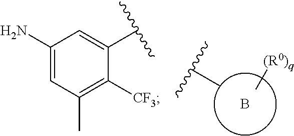

is

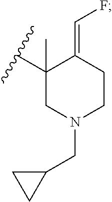 [SMILES: *C1(C)CN(CC2CC2)CC/C1=C\F]

and all of R^{1a}, R^{1b}, R^{2a} and R^{2b} are H.

In some embodiments,

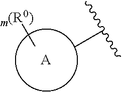

is

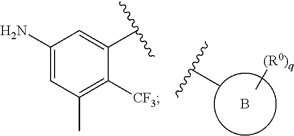

is

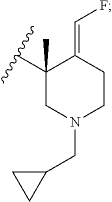 [SMILES: *[C@@]1(C)CN(CC2CC2)CC/C1=C\F]

and all of R^{1a}, R^{1b}, R^{2a} and R^{2b} are H.

In some embodiments,

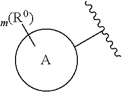

is

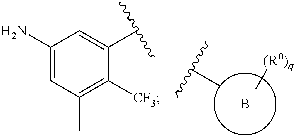

is

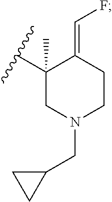 [SMILES: *[C@]1(C)CN(CC2CC2)CC/C1=C\F]

and all of R^{1a}, R^{1b}, R^{2a} and R^{2b} are H.

In some embodiments,

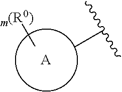

is

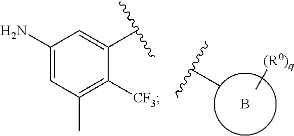

is

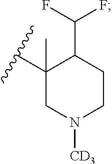 [SMILES: *C1(C)CN(C([2H])([2H])[2H])CCC1C(F)F]

all of R^{1a}, R^{1b}, R^{2a} and R^{2b} are H; and both R^{3a} and R^{3b} are H, wherein the H refers to deuterium.

In some embodiments,

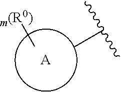

is

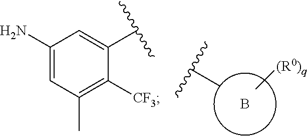

is

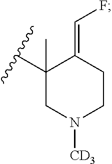 [SMILES: *C1(C)CN(C([2H])([2H])[2H])CC/C1=C\F]

all of R^{1a}, R^{1b}, R^{2a} and R^{2b} are H; and both R^{3a} and R^{3b} are H, wherein the H refers to deuterium.

In some embodiments,

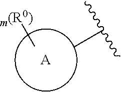

is

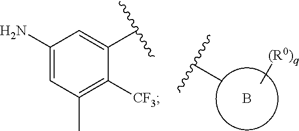

is

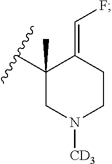 [SMILES: *[C@@]1(C)CN(C([2H])([2H])[2H])CC/C1=C\F]

all of R^{1a}, R^{1b}, R^{2a} and R^{2b} are H; and both R^{3a} and R^{3b} are H, wherein the H refers to deuterium.

In some embodiments,

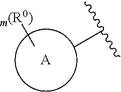

is

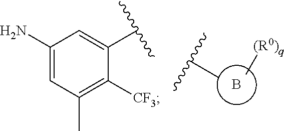

is

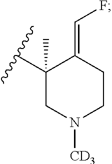 [SMILES: *[C@]1(C)CN(C([2H])([2H])[2H])CC/C1=C\F]

all of R^{1a}, R^{1b}, R^{2a} and R^{2b} are H; and both R^{3a} and R^{3b} are H, wherein the H refers to deuterium.

In some embodiments,

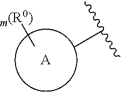

is

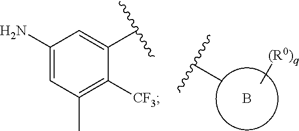

is

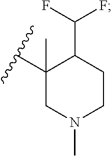 [SMILES: *C1(C)CN(C)CCC1C(F)F]

and all of R^{1a}, R^{1b}, R^{2a} and R^{2b} are H.

In some embodiments,

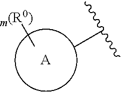

is

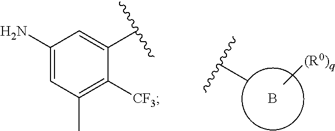

is

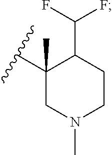 [SMILES: *[C@@]1(C)CN(C)CCC1C(F)F]

and all of R^{1a}, R^{1b}, R^{2a} and R^{2b} are H.

In some embodiments,

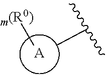

is

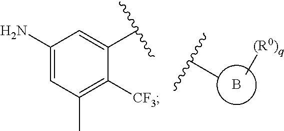

is

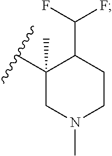 [SMILES: *[C@]1(C)CN(C)CCC1C(F)F]

and all of R^{1a}, R^{1b}, R^{2a} and R^{2b} are H.

In some embodiments,

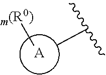

is

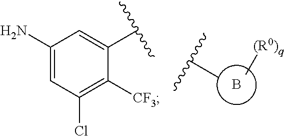

is

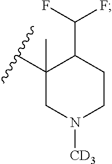 [SMILES: *C1(C)CN(C([2H])([2H])[2H])CCC1C(F)F]

all of R^{1a}, R^{1b}, R^{2a} and R^{2b} are H; and both R^{3a} and R^{3b} are H, wherein the H refers to deuterium.

In some embodiments,

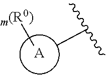

is

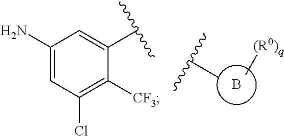

is

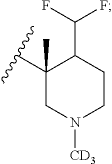 [SMILES: *[C@@]1(C)CN(C([2H])([2H])[2H])CCC1C(F)F]

all of R^{1a}, R^{1b}, R^{2a} and R^{2b} are H; and both R^{3a} and R^{3b} are H, wherein the H refers to deuterium.

In some embodiments,

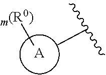

is

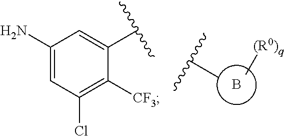

is

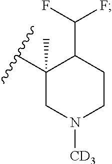 [SMILES: *[C@]1(C)CN(C([2H])([2H])[2H])CCC1C(F)F]

all of R^{1a}, R^{1b}, R^{2a} and R^{2b} are H; and both R^{3a} and R^{3b} are H, wherein the H refers to deuterium.

In some embodiments,

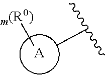

is

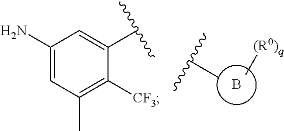

is

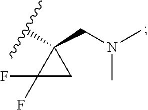 [SMILES: *[C@]1(CN(C)C)CC1(F)F]

R^{1a} is methyl; and all of R^{1b}, R^{2a} and R^{2b} are H.

In some embodiments, L¹ is a direct bond.

In some embodiments, moiety B is substituted or unsubstituted azetidinyl, substituted or unsubstituted diazaspiro[3.3]heptanyl, substituted or unsubstituted diazaspiro[3.4]octanyl, or substituted or unsubstituted diazaspiro[3.5]nonanyl; preferably substituted or unsubstituted 1,6-diazaspiro[3.3]heptan-6-yl, substituted or unsubstituted 2,5-diazaspiro[3.4]octan-2-yl, or substituted or unsubstituted 2,5-diazaspiro[3.5]nonan-2-yl.

In some embodiments, moiety B is

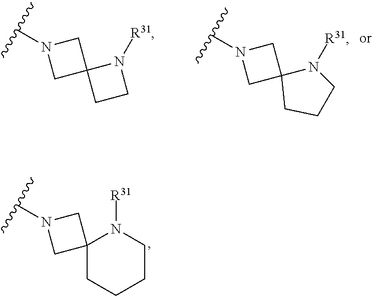

wherein R³¹ is H or substituted or unsubstituted C₁₋₄ alkyl.

In some embodiments, R³¹ is H, methyl, methyl-d3, or ethyl.

In some embodiments, the compound of formula (I) is a compound of formula (IIc):

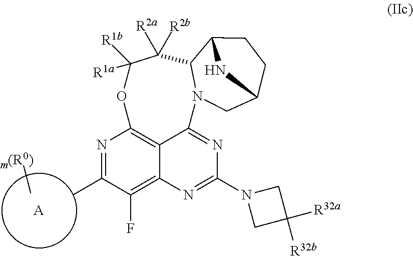

wherein
each of R^{32a} and R^{32b}, is, independently, H, unsubstituted or substituted C₁₋₄alkyl, unsubstituted or substituted amino, or unsubstituted or substituted heterocyclyl; and
the other variables are defined above.

In some embodiments, the compound of formula (I) is a compound of formula (IIc):

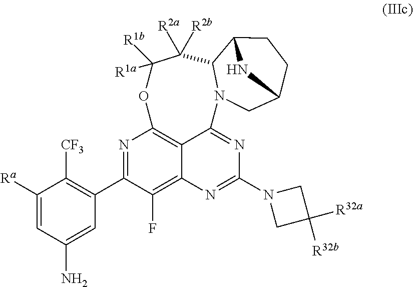

wherein
R^{a} is methyl, methyl-d3, or Cl; and
the other variables are defined above.

In some embodiments, R^{32a} is dimethylamino, ethyl(methyl)amino, diethylamino, azetidine-1-yl, 3-fluoroazetidin-1-yl, 3-methoxyazetidin-1-yl, pyrrolidin-1-yl, 2-fluorrpyrrolidin-1-yl, 2-methoxypyrrolidin-1-yl, piperidin-1-yl, 2-fluoropiperidin-1-yl, 3-fluoropiperidin-1-yl, 2-methoxypiperidin-1-yl, 3-methoxypiperidin-1-yl, morpholino, 3-oxa-6-azabicyclo[3.1.1]heptan-6-yl, 6-oxa-3-azabicyclo[3.1.1]heptan-3-yl, (1S,4S)-2-oxa-5-azabicyclo[2.2.1]heptan-5-yl, (1R,4R)-2-oxa-5-azabicyclo[2.2.1]heptan-5-yl, 3-oxa-8-azabicyclo[3.2.1]octan-8-yl, 8-azabicyclo[3.2.1]octan-8-yl, 8-oxa-3-azabicyclo[3.2.1]octan-3-yl, 3-azabicyclo[3.1.1]heptan-3-yl, 2-oxa-6-azaspiro[3.3]heptan-6-yl, or 6-azabicyclo[3.1.1]heptan-6-yl.

In some embodiments, R^{32b} is H, methyl, methyl-d3, fluoromethyl, methoxymethyl, or ethyl; preferably H, methyl, or methyl-d3.

In some embodiments, the

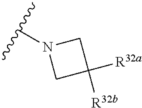

is

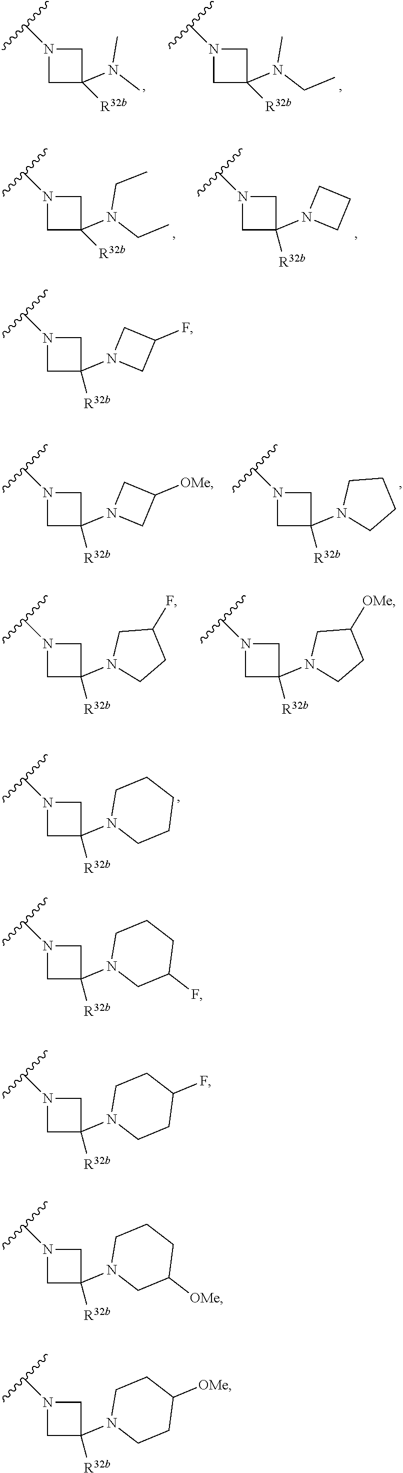

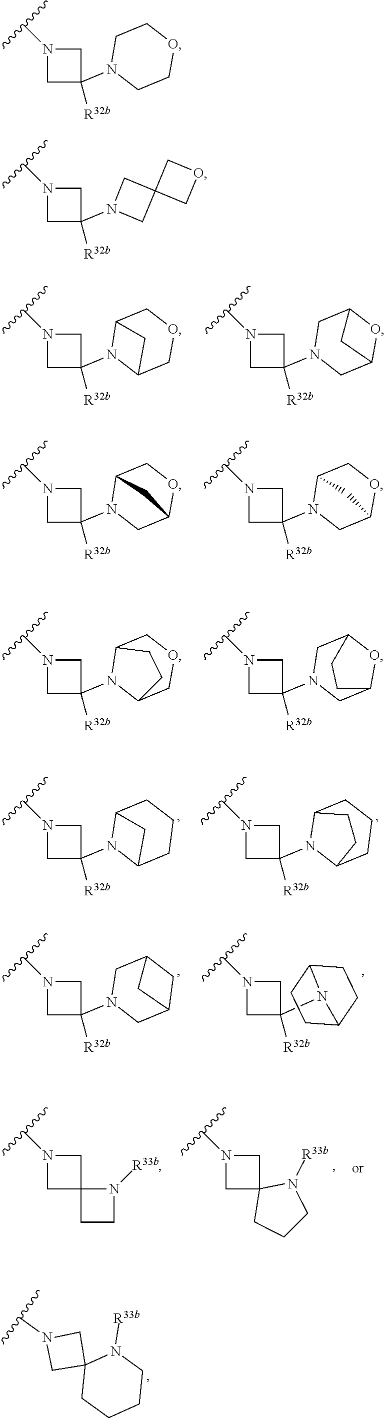

wherein R^{32b} is H, methyl, methyl-d3, fluoromethyl, methoxymethyl, or ethyl; and R^{33b} is H, methyl, methyl-d3 or ethyl. In some embodiments, the

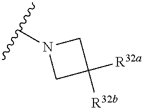

is

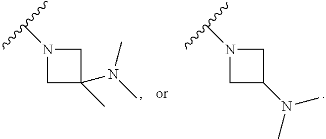

In some embodiments, the

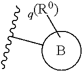

is

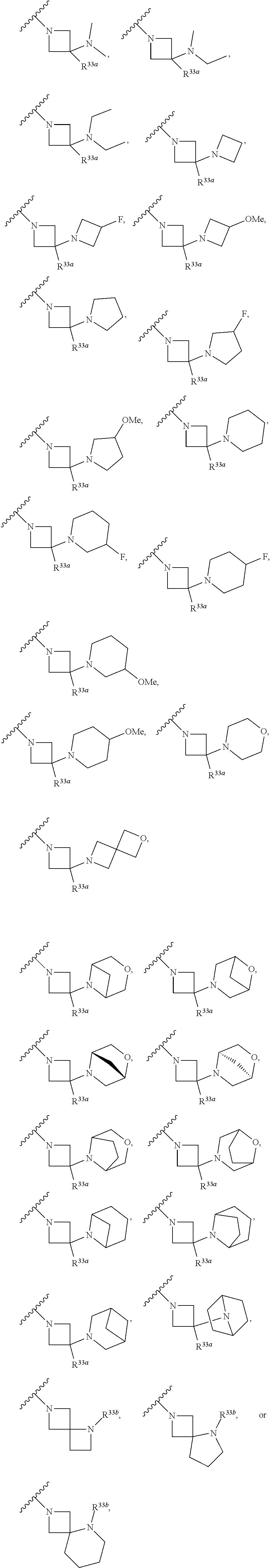

wherein R^{33a} is H, methyl, methyl-d3, fluoromethyl, methoxymethyl, or ethyl; and
R^{33b} is H, methyl, methyl-d3 or ethyl.

In some embodiments, each of R^{1a}, R^{1b}, R^{2a} and R^{2b}, is, independently, H, or methyl, or R^{1a} and R^{1b}, together form an oxo.

In some embodiments, all of R^{1a}, R^{1b}, R^{2a} and R^{2b} are H.

In some embodiments, ring A is substituted or unsubstituted phenyl, or substituted or unsubstituted naphthyl; preferably ring A is phenyl or naphthyl, wherein said phenyl and naphthyl are optionally substituted with 1, 2, 3 or 4 substituents selected from amino, —Cl, —F, cyano, ethyl, methyl, trifluoromethyl, cyclopropyl, hydroxy, or 1,1-difluoroethyl.

In some embodiments, ring A is phenyl optionally substituted with 1, 2, 3 or 4 substituents independently selected from amino, hydroxyl, —Cl, —F, ethyl, methyl, methyl-d3, trifluoromethyl, or cyclopropyl; preferably aminophenyl optionally substituted with 1, 2, 3 or 4 substituents independently selected from —Cl, —F, ethyl, methyl, methyl-d3, trifluoromethyl, or cyclopropyl.

In some embodiments,

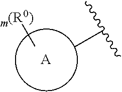

is

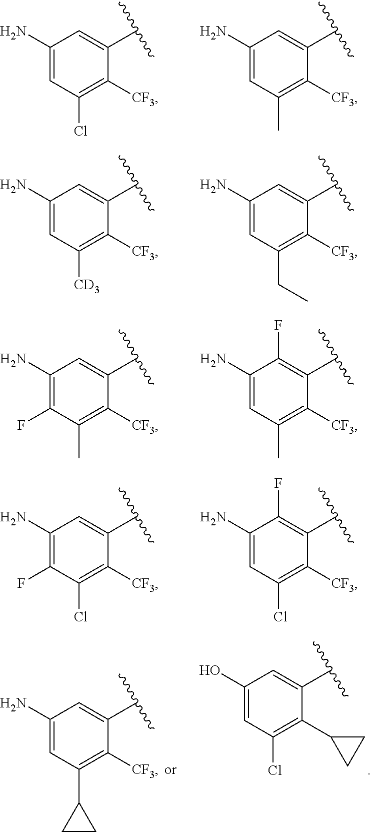

In some embodiments, ring A is naphthyl optionally substituted with 1, 2, 3 or 4 substituents independently selected from —Cl, —F, ethenyl, ethyl, cyclopropyl, or hydroxy; preferably hydroxylnaphthyl optionally substituted with 1, 2, 3 or 4 substituents independently selected from —Cl, —F, ethenyl, ethyl, or cyclopropyl.

In some embodiments,

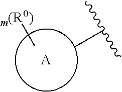

is

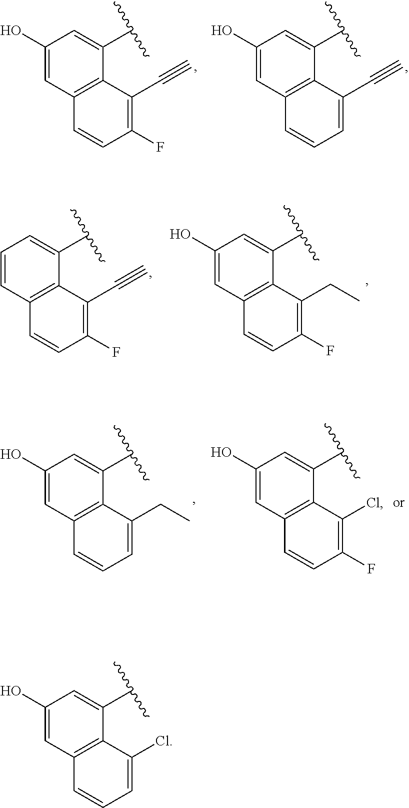

In some embodiments,

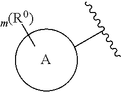

is

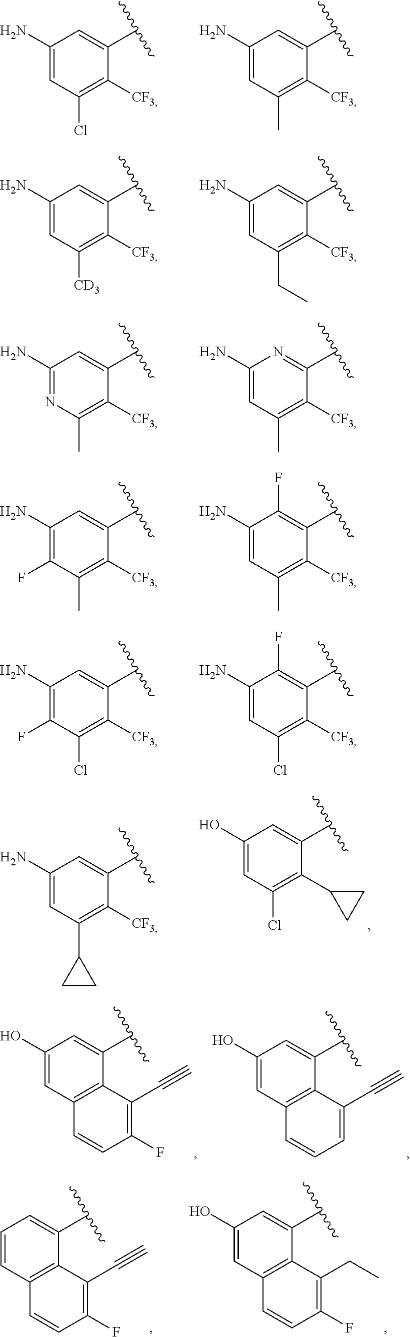

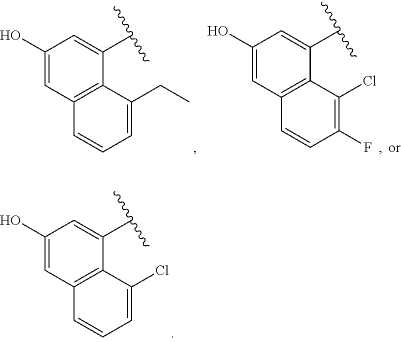 [SMILES: *c1cc(O)cc2cccc(CC)c12 ; *c1cc(O)cc2ccc(F)c(Cl)c12 ; *c1cc(O)cc2cccc(Cl)c12]

In some embodiments, the compound is one of the compounds in Table 2.

Provided here is a pharmaceutical composition comprising a compound provided herein, or a pharmaceutically acceptable salt, tautomer, stereoisomer, enantiomer, or atropisomer thereof, and a pharmaceutically acceptable carrier, excipient or vehicle.

Provided here is a method for inhibiting the activity of KRAS mutant protein in a cell, comprising contacting said cell with a compound provided herein, or a pharmaceutically acceptable salt, tautomer, stereoisomer, enantiomer, or atropisomer thereof, optionally wherein the KRAS mutant protein is KRAS G12D mutant protein.

Provided here is a method for treatment or prevention of cancer, the method comprising administering to a subject in need thereof a compound provided herein, or a pharmaceutically acceptable salt, tautomer, stereoisomer, enantiomer, or atropisomer thereof, optionally wherein the cancer is mediated by KRAS mutation; preferably KRAS G12D mutation. Provided here is a method for the treatment or prevention of a cancer, the methods comprising administering to a subject in need thereof a compound provided herein.

Provided here is a method of modulating activity of KRAS G12D, comprising contacting said cell with a compound provided herein, or a pharmaceutically acceptable salt, tautomer, stereoisomer, enantiomer, or atropisomer thereof.

Provided herein is a kit for treating cancer, the kit comprising (a) a pharmaceutical composition comprising a compound provided herein; and (b) instructions for administration of the pharmaceutical composition comprising the KRAS G12D inhibitor provided herein to treat cancer in an individual.

### NUMBERED EMBODIMENTS

1. A compound having Formula (I):

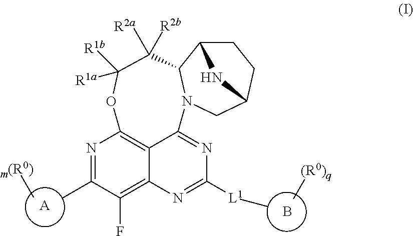

or a pharmaceutically acceptable salt, tautomer, stereoisomer, enantiomer, or atropisomer thereof,
wherein
ring A is unsubstituted or substituted aryl, or unsubstituted or substituted heteroaryl;
moiety B is unsubstituted or substituted cycloalkyl, or unsubstituted or substituted heterocyclyl;
L¹ is a direct bond, or —O—R^{a}—, wherein said R^{a} is, absent or unsubstituted or substituted C₁₋₄ alkylene;
each of R⁰ is, independently, H, halogen, —CN, —OH, unsubstituted or substituted C₁₋₄alkyl, unsubstituted or substituted C₁₋₄alkoxy, unsubstituted or substituted C₁₋₄ alkenyl, unsubstituted or substituted C₃₋₅cycloalkyl, unsubstituted or substituted 3-member to 6-member heterocyclyl, or unsubstituted or substituted amino; or one or more pairs of the R⁰ groups, together with the atom(s) to which they are attached to, form unsubstituted or substituted cycloalkyl, unsubstituted or substituted alkenyl, unsubstituted or substituted heterocyclyl, unsubstituted or substituted aryl, or unsubstituted or substituted heteroaryl;
each of R^{1a}, R^{1b}, R^{2a} and R^{2b}, is, independently, H, halogen, unsubstituted or substituted C₁₋₃alkyl, or R^{1a} and R^{1b}, together form an oxo or a substituted or unsubstituted cyclopropyl;
each of m, and q is, independently, an integer from 0 to the maximum number of the substituent groups allowed on rings A, and B, respectively;
provided said compound is not the compound of Table 1.
2. The compound of embodiment 1, wherein L¹ is —O—.
3. The compound of embodiment 1, wherein the compound of formula (I) is a compound of formula (IIa):

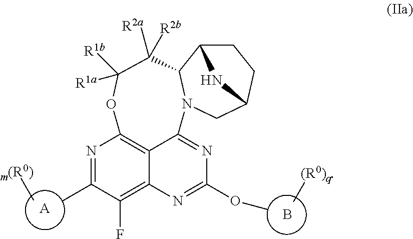

4. The compound of any one of embodiments 1-3, wherein moiety B is substituted or unsubstituted cycloalkyl, or substituted or unsubstituted heterocyclyl containing 1-3 heteroatoms independently selected from N or O.
5. The compound of any one of embodiments 1-3, wherein moiety B is substituted or unsubstituted 4- to 6-membered cycloalkyl, or substituted or unsubstituted heterocyclyl containing one oxygen atom.
6. The compound of any one of embodiments 1-4, wherein moiety B is substituted or unsubstituted tetrahydro-2H-pyran-yl or substituted or unsubstituted furanyl; preferably tetrahydro-2H-pyran-yl or furanyl, wherein said each of tetrahydro-2H-pyran-yl or furanyl is optionally substituted with C₁₋₄ alkylamino or heterocyclyl containing one or more nitrogen atoms; more preferably tetrahydro-2H-pyran-yl substituted with dimethylamino, or furanyl substituted with dimethylamino.
7. The compound of any one of embodiments 1-6, wherein moiety B is

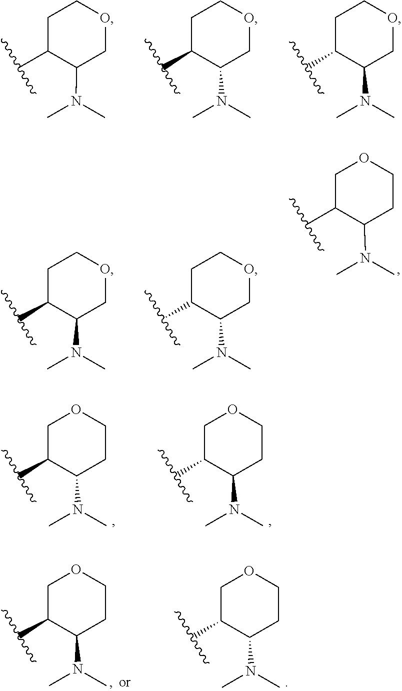

8. The compound of any one of embodiments 1-6, wherein moiety B is

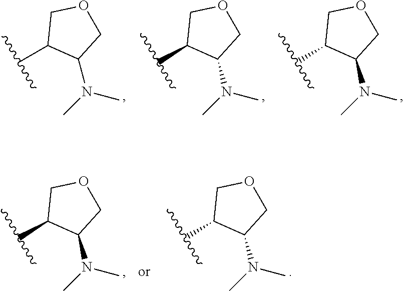

9. The compound of any one of embodiments 1-5, wherein moiety B is substituted or unsubstituted cyclobutyl, substituted or unsubstituted cyclopentyl, substituted or unsubstituted cycloheptyl; preferably substituted or unsubstituted 2-dimethylamino-cyclobutyl, substituted or unsubstituted 2-heterocyclyl-cyclobutyl, substituted or unsubstituted 2-dimethylamino-cyclopentyl, substituted or unsubstituted 2-heterocyclyl-cyclopentyl, substituted or unsubstituted 2-dimethylamino-cycloheptyl, substituted or unsubstituted 2-heterocyclyl-cycloheptyl, wherein said heterocyclyl is the heterocyclyl containing one or more nitrogen atoms; more preferably 2-dimethylamino-cyclobutyl, 2-dimethylamino-cyclopentyl, or 2-dimethylamino-cycloheptyl, wherein said each of 2-dimethylamino-cyclobutyl, 2-dimethylamino-cyclopentyl and 2-dimethylamino-cycloheptyl is optionally substituted with H, F, methoxy, methyl, methyl-d3, methoxy-d3, CF₂H, or CF₃.
10. The compound of any one of embodiments 1-5 and 9, wherein moiety B is

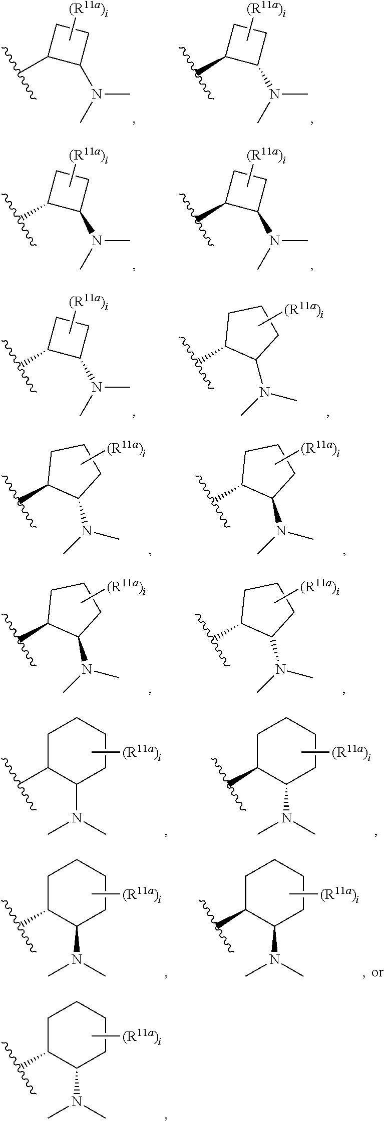

wherein R^{11a} is H, F, methoxy, methyl, methyl-d3, methoxy-d3, CF₂H, or CF₃; and i is 0, 1, 2, 3, or 4.
11. The compound of any one of embodiments 1-5 and 9, wherein moiety B is

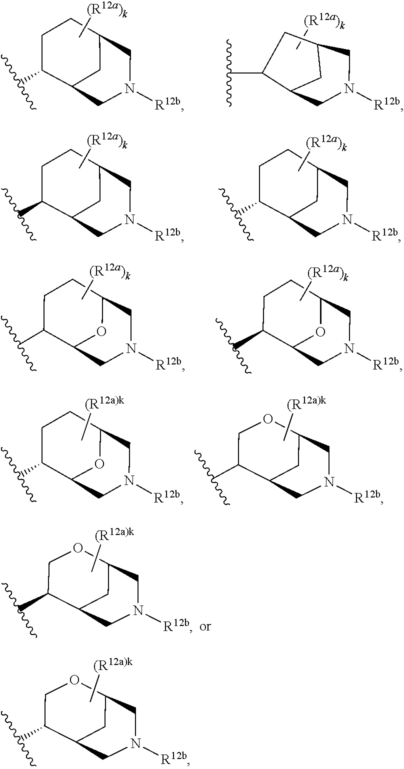

wherein R^{12a} is H, F, methoxy, methyl, methyl-d3, methoxy-d3, difluoromethyl, or trifluoromethyl;
R^{12b} is C₁₋₃alkyl optionally substituted with one or more substituents selected from methyl, methyl-d3, F, methoxy, or alkenyl; and
k is 0, 1, or 2.
12. The compound of embodiment 11 wherein moiety B is

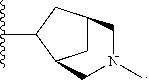 [SMILES: *C1C[C@@H]2C[C@H]1CN(C)C2]

13. The compound of any one of embodiments 1-5, wherein

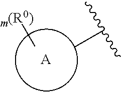

is

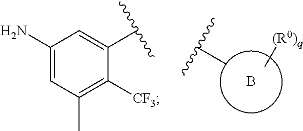

is

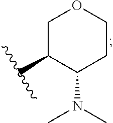 [SMILES: *[C@H]1COCC[C@@H]1N(C)C]

and all of R^{1a}, R^{1b}, R^{2a} and R^{2b} are H.
14. The compound of embodiment 1, wherein L¹ is a —O—C(R^{3a})(R^{3b})—, wherein said each of R^{3a} and R^{3b} is, independently, H, F, unsubstituted or substituted C₁₋₂alkyl.
15. The compound of embodiment 1, wherein the compound of formula (I) is a compound of formula (IIb):

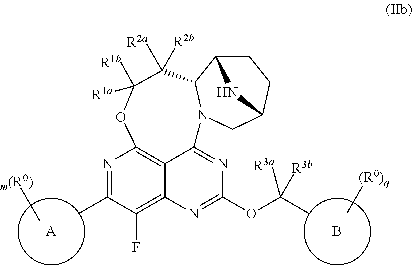

wherein
each of R^{3a} and R^{3b}, is, independently, H, F, unsubstituted or substituted C₁₋₂alkyl.
16. The compound of embodiment 15 wherein R^{3a} is H, methyl, methyl-d3, fluoromethyl, difluoromethyl, or trifluoromethyl; and R^{3b} is H.
17. The compound of any one of embodiments 15-16, wherein both R^{3a} and R^{3b} are H.
18. The compound of any one of embodiments 15-16, wherein R^{3a} is methyl; R^{3b} is H.
19. The compound of any one of embodiments 15, 16 and 18, wherein the carbon atom which R^{3a} and R^{3b} connect to is R-configuration.
20. The compound of any one of embodiments 15, 16 and 18, wherein the carbon atom which R^{3a} and R^{3b} connect to is S-configuration.
21. The compound of embodiment 15, wherein the compound of formula (IIb) is a compound of formula (IIIa):

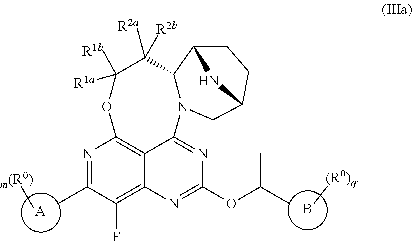

22. The compound of embodiment 15, wherein the compound of formula (IIIa) is a compound of formula (IVa):

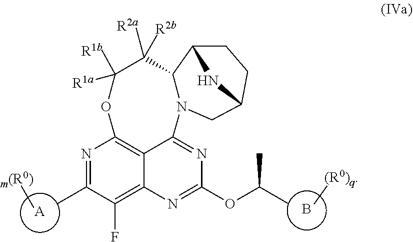

23. The compound of any one of embodiments 14-22, wherein moiety B is substituted or unsubstituted C₃₋₆cycloalkyl, or substituted or unsubstituted 4- to 6-membered heterocyclyl containing one or more hetero atoms; preferably cyclopropyl or substituted or unsubstituted heterocyclyl containing one or more heteroatoms selected from N or O, wherein said cyclopropyl is optionally substituted with one or more substituents selected from halogen, or substituted or unsubstituted alkyl.
24. The compound of any one of embodiments 14-23, wherein moiety B is

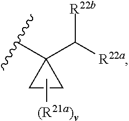

wherein
each of R^{21a} is, independently, H or halogen;
each of R^{22a} and R^{22b} is, independently, H, substituted or unsubstituted alkyl, substituted or unsubstituted amino or substituted or unsubstituted heterocyclyl, or R^{22a} and R^{22b}, together with the atom which R^{22a} and R^{22b} connect with, form a substituted or unsubstituted heterocyclyl; and
v is 0, 1, 2, 3, or 4.
25. The compound of any one of embodiments 14-23, wherein moiety B is

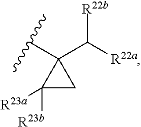

wherein
each of R^{23a} and R^{23b} is, independently, H or halogen;
each of R^{22a} and R^{22b} is, independently, H, substituted or unsubstituted alkyl, substituted or unsubstituted amino or substituted or unsubstituted heterocyclyl, or R^{22a} and R^{22b}, together with the atom which R^{22a} and R^{22b} connect with, form a substituted or unsubstituted heterocyclyl.
26. The compound of embodiment 25, wherein the carbon atom of cyclopropyl attached to the branch containing R^{22a} and R^{22b} is R-configuration.
27. The compound of embodiment 25, wherein the carbon atom of cyclopropyl attached to the branch containing R^{22a} and R^{22b} is S-configuration.
28. The compound of any one of embodiments 25-27, wherein each of R^{22a} and R^{22b} is, independently, H, substituted or unsubstituted amino, or substituted or unsubstituted C₁₋₃alkyl; preferably each of R^{22a} and R^{22b} is, independently, H, methyl, or dimethylamino.
29. The compound of any one of embodiments 25-28, wherein moiety B is

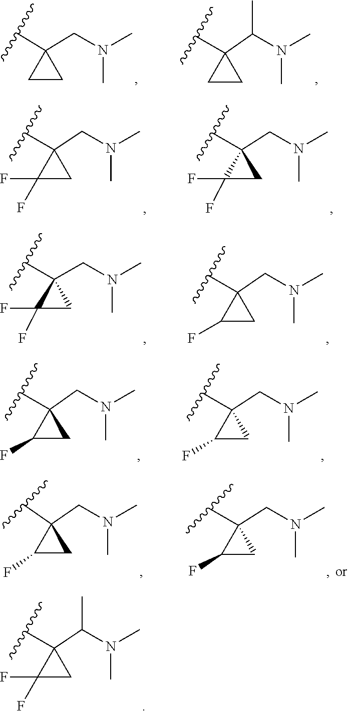

30. The compound of any one of embodiments 24-27, wherein R^{22b} is H or methyl and R^{22a} is substituted or unsubstituted heterocyclyl; preferably R^{22b} is H or methyl and R^{22a} is substituted or unsubstituted azetidinyl, substituted or unsubstituted pyrrolidinyl, substituted or unsubstituted piperidinyl, substituted or unsubstituted 3-azabicyclo[3.1.0]hexanyl, substituted or unsubstituted 2,5-dihydro-1H-pyrrolyl, substituted or unsubstituted 6-azaspiro[2.5]octanyl, substituted or unsubstituted 1,2,3,6-tetrahydropyridinyl, substituted or unsubstituted morpholinyl, substituted or unsubstituted 2-oxa-6-azaspiro[3.3]heptanyl, substituted or unsubstituted (1R,4R)-2-oxa-5-azabicyclo[2.2.1]heptanyl, substituted or unsubstituted (1S,4S)-2-oxa-5-azabicyclo[2.2.1]heptanyl; more preferably R^{22b} is H or methyl and R^{22a} is substituted or unsubstituted azetidine-1-yl, substituted or unsubstituted pyrrolidine-1-yl, substituted or unsubstituted piperidin-1-yl, substituted or unsubstituted 2,5-dihydro-1H-pyrrol-1-yl, substituted or unsubstituted 6-azaspiro[2.5]octan-6-yl, substituted or unsubstituted 1,2,3,6-tetrahydropyridin-1-yl, substituted or unsubstituted morpholin-4-yl, substituted or unsubstituted 2-oxa-6-azaspiro[3.3]heptan-6-yl, substituted or unsubstituted (1R,4R)-2-oxa-5-azabicyclo[2.2.1]heptan-5-yl, substituted or unsubstituted (1S,4S)-2-oxa-5-azabicyclo[2.2.1]heptan-5-yl, substituted or unsubstituted 2-azaspiro[3.3]heptane-2-yl, substituted or unsubstituted 2-azabicyclo[2.2.1]heptan-2-yl, substituted or unsubstituted 3-azabicyclo[3.1.1]heptan-3-yl, substituted or unsubstituted 2-oxa-6-azaspiro[3.4]octan-6-yl, substituted or unsubstituted 2-oxa-7-azaspiro[4.4]nonan-7-yl, substituted or unsubstituted 7-oxa-1-azaspiro[4.4]nonan-1-yl, or substituted or unsubstituted 7-azaspiro[3.5]nonan-7-yl.
31. The compound of any one of embodiments 24-27 or 30, wherein moiety B is

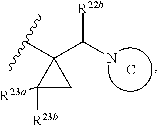

wherein ring C is substituted or unsubstituted 4- to 10-membered heterocyclyl optionally containing one or more additional heteroatom selected from N, O, or S; preferably substituted or unsubstituted 4- to 7-membered heterocyclyl optionally containing one or more additional heteroatom selected from N, O, or S.
32. The compound of embodiment 31, wherein moiety B is

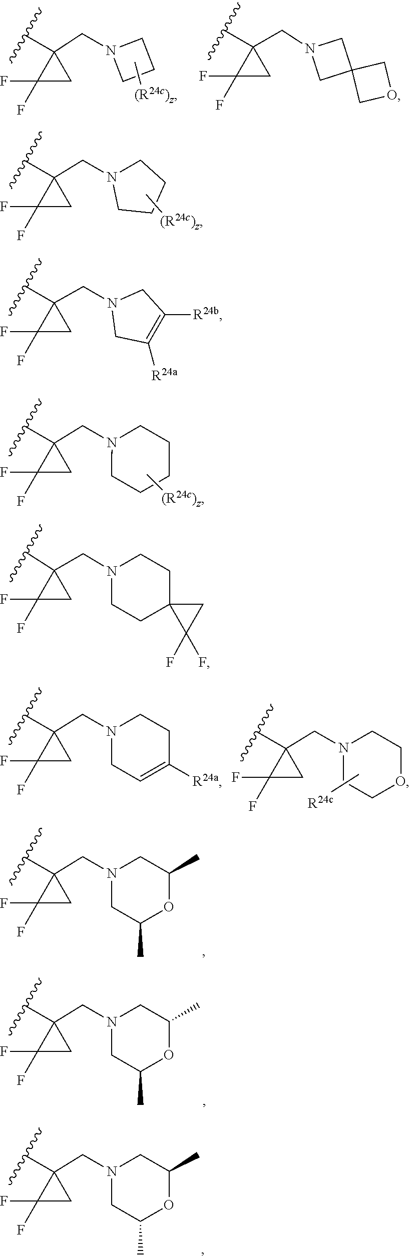

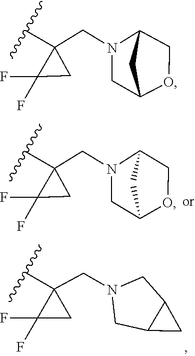

wherein each of R^{24a} and R^{24b} is, independently, H, methyl, ethyl, methyl-d3, F, or Cl; R^{24c} is H, F, methoxy, methyl, methyl-d3, ethyl, difluoromethyl, or trifluoromethyl; and z is 0, 1, or 2.
33. The compound of embodiment 31, wherein moiety B is

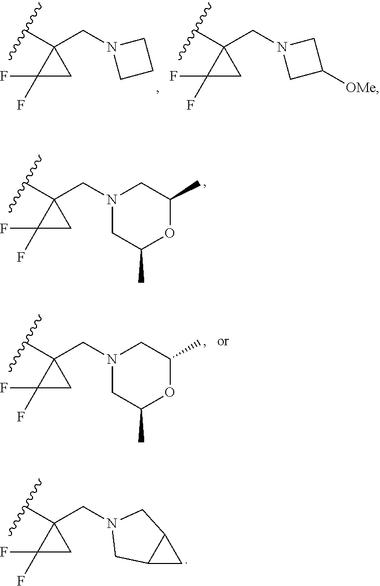

34. The compound of embodiment 31, wherein moiety B is

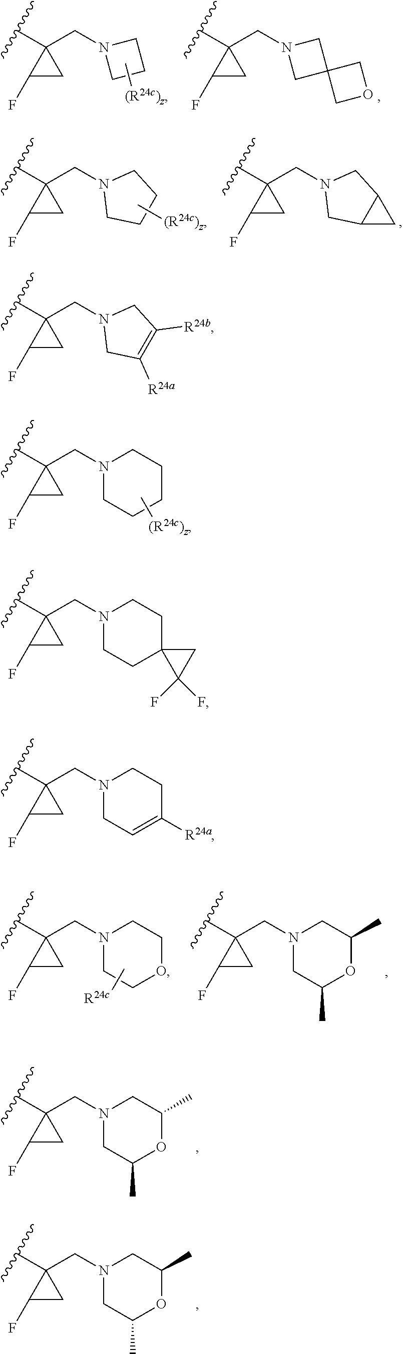

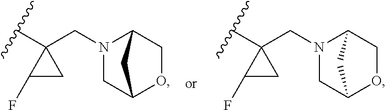 [SMILES: *C1(CN2C[C@@H]3C[C@H]2CO3)CC1F ; *C1(CN2C[C@H]3C[C@@H]2CO3)CC1F]

wherein each of R^{24a} and R^{24b} is, independently, H, methyl, ethyl, methyl-d3, F, or Cl; R^{24c} is H, F, methoxy, methyl, methyl-d3, ethyl, difluoromethyl, or trifluoromethyl; and z is 0, 1, or 2.
35. The compound of embodiment 31, wherein moiety B is

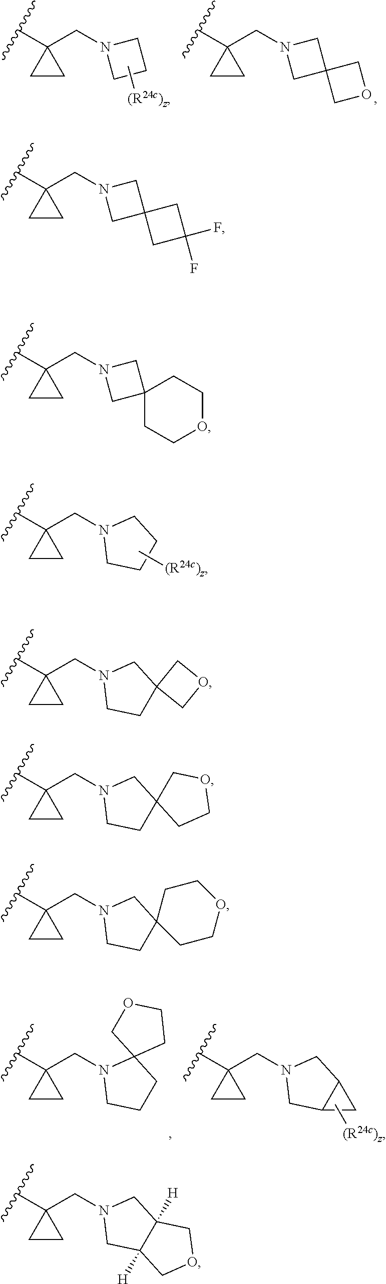

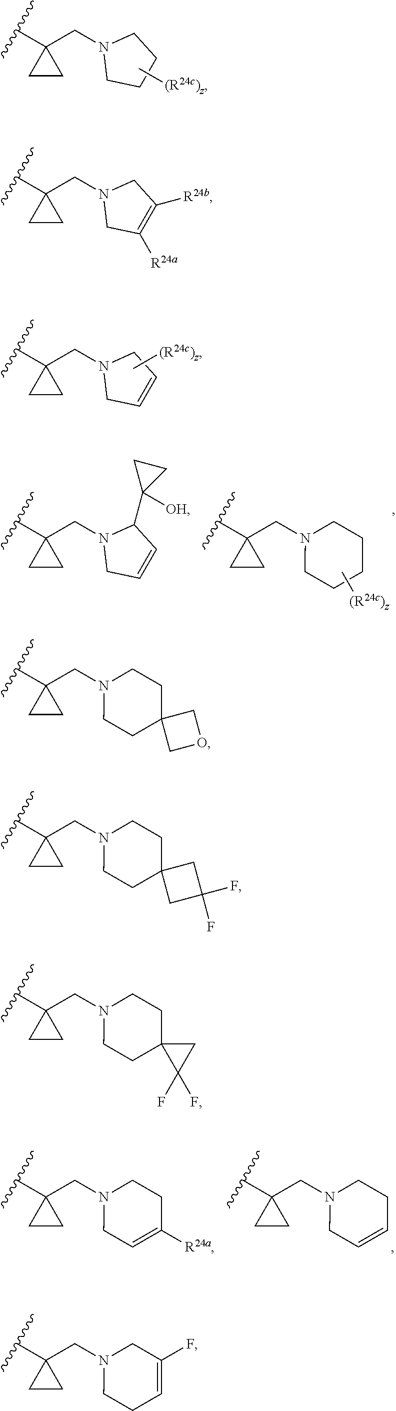

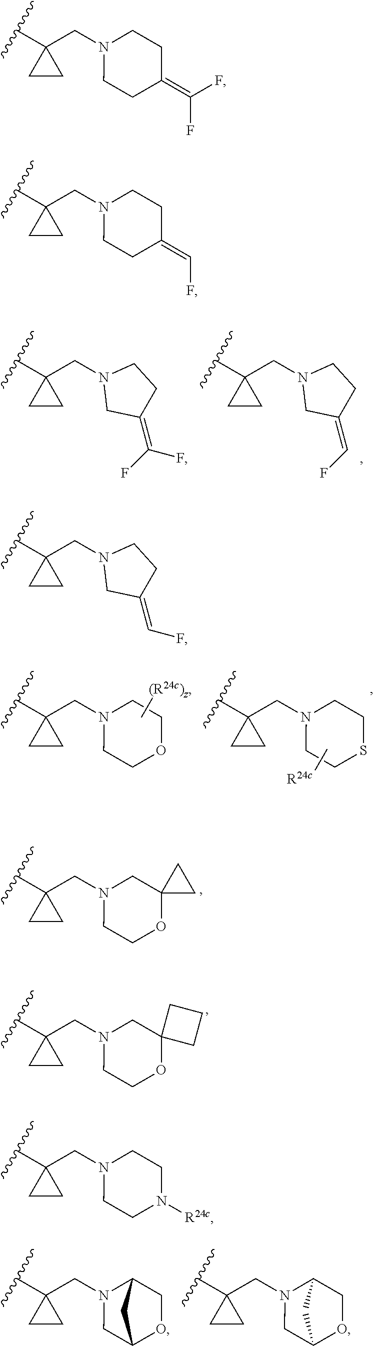

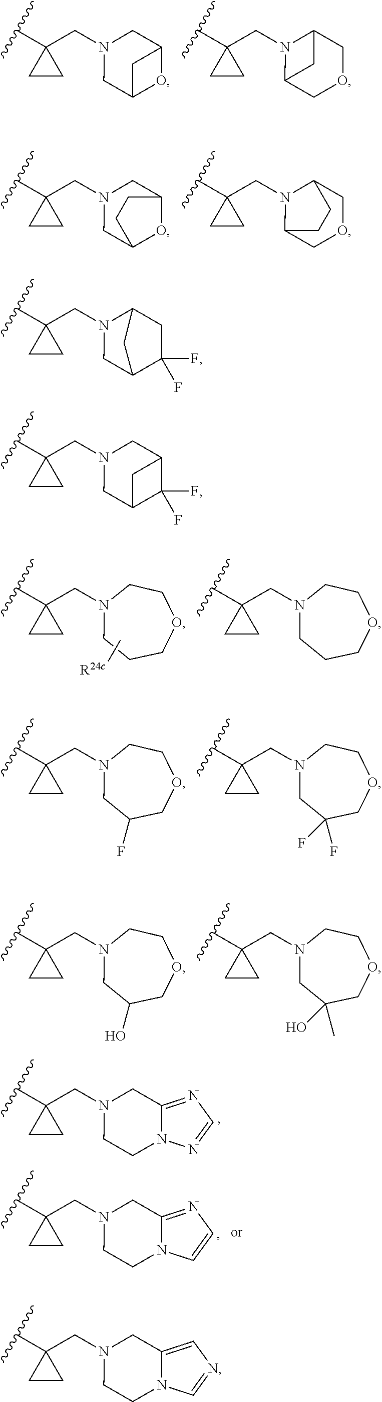

wherein each of R^{24a} and R^{24b} is, independently, H, methyl, ethyl, methyl-d3, F, or Cl; and R^{24c} is H, F, hydroxy, cyano, methoxy, methyl, methyl-d3, ethyl, fluoromethyl, difluoromethyl, trifluoromethyl, hydroxymethyl, difluoromethoxy, cyclopropyl, or oxetanyl.
36. The compound of embodiment 31, wherein moiety B is

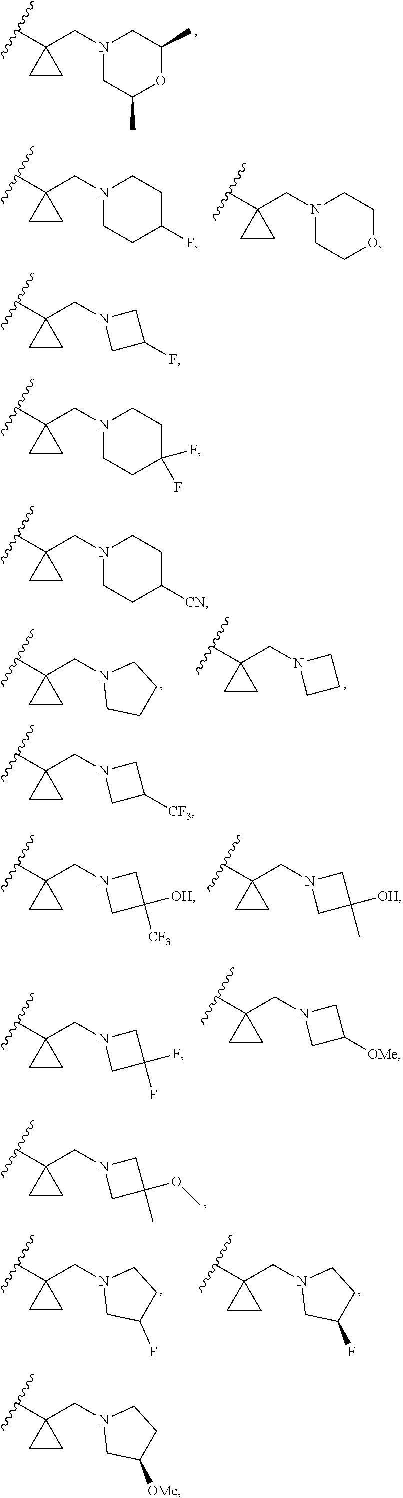

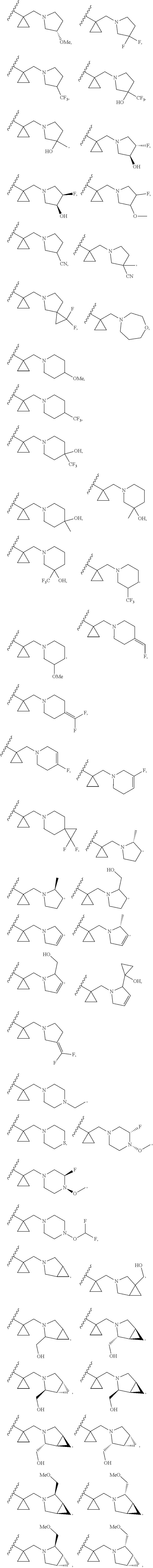

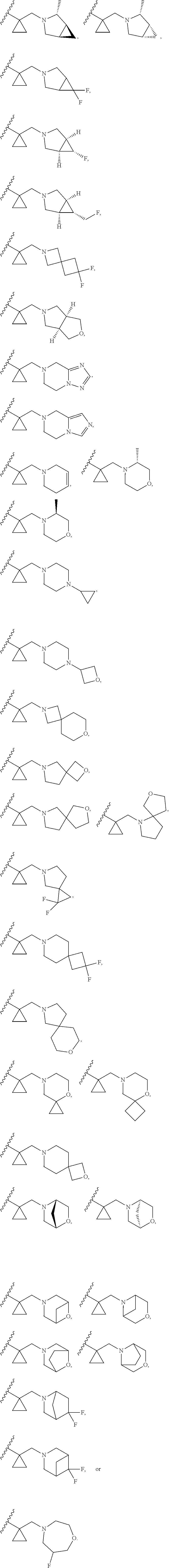

37. The compound of any one of embodiments 24-27, wherein R^{22a} and R^{22b}, together with the atom which R^{22a} and R^{22b} connect with, form a substituted or unsubstituted 5- to 6-membered heterocyclyl; preferably substituted or unsubstituted pyrrolidinyl, substituted or unsubstituted piperidinyl, or substituted or unsubstituted morpholinyl; more preferably pyrrolidinyl, piperidinyl, or morpholinyl, wherein said each of pyrrolidinyl, piperidinyl, and morpholinyl is optionally substituted with H, halogen, substituted or unsubstituted alkoxy, or substituted or unsubstituted alkyl.
38. The compound of any one of embodiments 24-27 and 37, wherein moiety B is

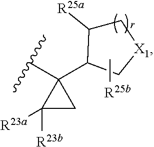

wherein R^{25a} is C₁₋₃alkyl optionally substituted with one or more substituents selected from methyl, methyl-d3, F or alkenyl;
R^{25b} is H, F, methoxy, methyl, difluoromethyl, or trifluoromethyl;
X¹ is O or C; and
r is, 1 or 2.
39. The compound of embodiment 38, wherein moiety B is

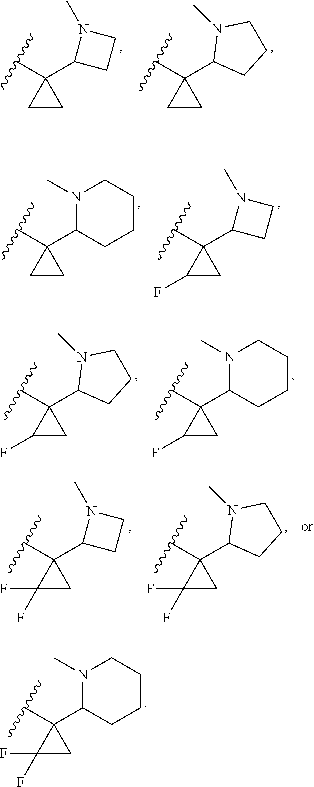

40. The compound of any one of embodiments 14-23, wherein moiety B is substituted or unsubstituted 4- to 6-membered heterocyclyl containing one or two heteroatoms selected from N or O.
41. The compound of any one of embodiments 14-23 and 40, wherein moiety B is substituted or unsubstituted 4- to 6-membered heterocyclyl containing one oxygen atom; preferably substituted or unsubstituted oxetanyl, substituted or unsubstituted tetrahydrofuranyl, or substituted or unsubstituted tetrahydro-2H-pyranyl; more preferably substituted or unsubstituted oxetan-2-yl, substituted or unsubstituted tetrahydrofuran-3-yl, substituted or unsubstituted tetrahydrofuran-2-yl, substituted or unsubstituted tetrahydro-2H-pyran-4-yl, or substituted or unsubstituted tetrahydro-2H-pyran-3-yl.
42. The compound of any one of embodiments 14-23 and 40-41, wherein moiety B is 3-dimethylamino-oxetan-2-yl, 4-dimethylamino-tetrahydrofuran-3-yl, 3-dimethylamino-tetrahydrofuran-2-yl, 3-dimethylamino-tetrahydro-2H-pyran-4-yl, or 4-dimethylamino-tetrahydro-2H-pyran-3-yl, wherein said each of 3-dimethylamino-oxetan-2-yl, 4-dimethylamino-tetrahydrofuran-3-yl, 3-dimethylamino-tetrahydrofuran-2-yl, 3-dimethylamino-tetrahydro-2H-pyran-4-yl, and 4-dimethylamino-tetrahydro-2H-pyran-3-yl is optionally substituted with methyl, F, hydroxy, methoxy, or difluoromethyl.
43. The compound of any one of embodiments 14-23 and 40-42, wherein moiety B is

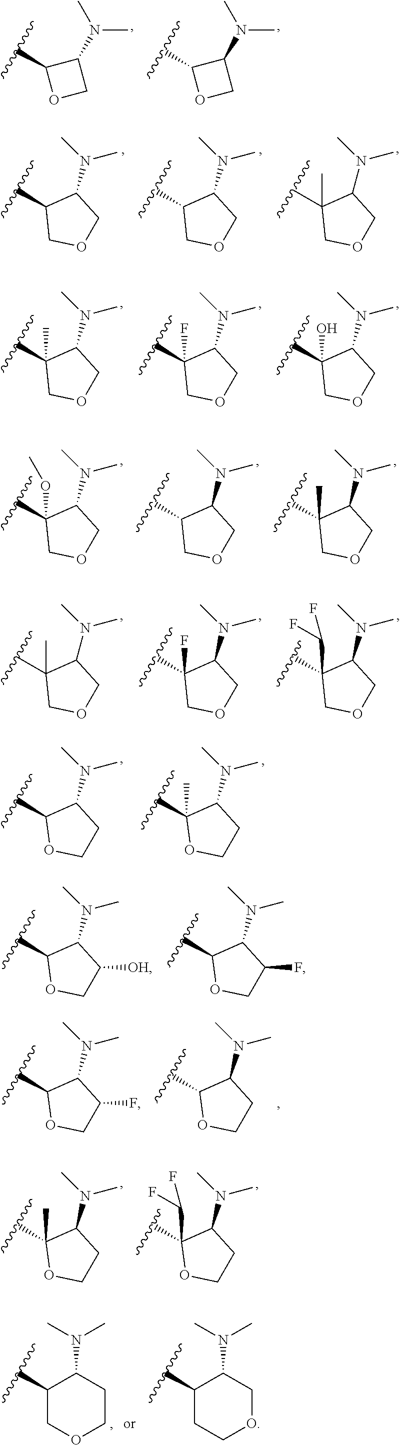

44. The compound of any one of embodiments 14-23, wherein moiety B is substituted or unsubstituted pyrrolidinyl; preferably substituted or unsubstituted pyrrolidine-2-yl optionally substituted with one or more substituents selected from H, F, methyl, difluoromethyl, ethyl, 2-fluoroethyl, 2-(methoxy-d3)ethyl, 1-allyl-2-methyl, or methoxy.
45. The compound of any one of embodiments 14-23 and 44, wherein moiety B is

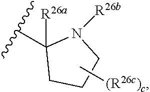

wherein
R^{26a} is H, methyl, or difluoromethyl;
R^{26b} is methyl, methyl-d3, ethyl, 2-fluoroethyl, 2-(methoxy-d3)ethyl, 1-allyl-2-methyl cyclopropylmethyl, oxetanyl, tetrahydrofuranyl, or tetrahydro-2H-pyranyl;
each R^{26c} is, independently, H, F or methoxy, or one pair of R^{26c}, together with the atom(s) to which they are attached form a substituted or unsubstituted cyclopropyl; and
c is 0, 1, or 2.
46. The compound of any one of embodiments 14-23 and 45, wherein the carbon atom which R^{26a} connects to is S-configuration.
47. The compound of any one of embodiments 14-23 and 45, wherein the carbon atom which R^{26a} connects to is R-configuration.
48. The compound of any one of embodiments 14-23 and 45-47, wherein moiety B is

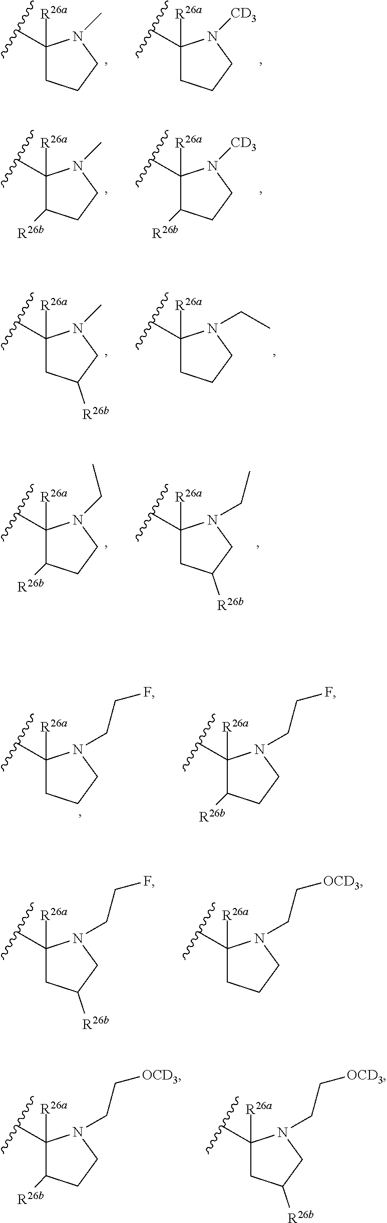

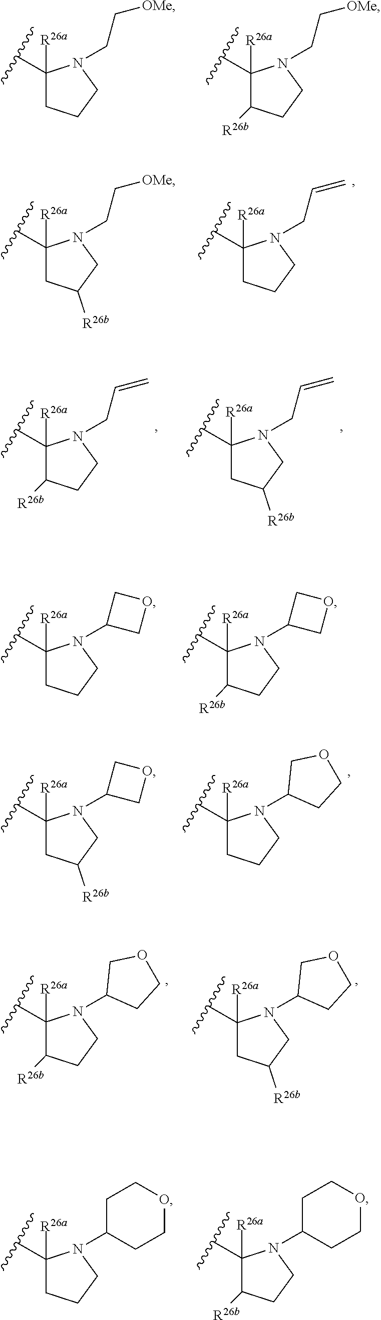

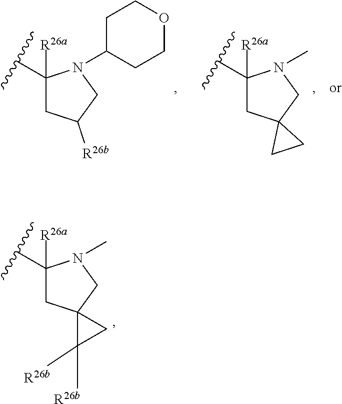

wherein R^{26a} is H, methyl, or difluoromethyl; each R^{26b} is, independently, methoxy, methoxy-d3, fluoro, methyl, fluoromethyl, or difluoromethyl.
49. The compound of any one of embodiments 14-23 and 45-48, wherein moiety B is

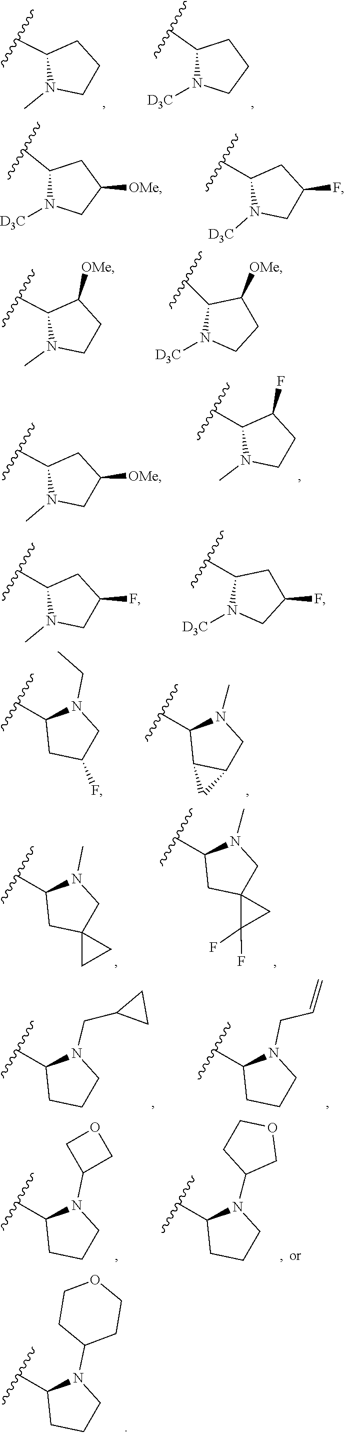

50. The compound of any one of embodiments 14-23, wherein moiety B is substituted or unsubstituted 6-membered heterocyclyl containing one or two heteroatoms selected from O or N, provided at least one heteroatom is N; preferably moiety B is substituted or unsubstituted piperidinyl, substituted or unsubstituted morpholinyl, substituted or unsubstituted piperazinyl.
51. The compound of any one of embodiments 14-23 and 50, wherein moiety B is

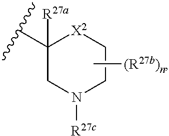

wherein
X² is —CHF—, —CF₂—, —CH(CH₃)—, N(CH₃)—, —CH(OCH₃)—, —CH(CHF₂)—, —C(═CHF)—, or —O—;
R^{27a} is methyl, ethyl, difluoromethyl, or trifluoromethyl;
R^{27b} is H, F, methyl, or methoxy;
R^{21c} is C₁₋₃alkyl, oxetanyl, tetrahydrofuranyl, or tetrahydropyranyl, wherein said C₁₋₃alkyl is optionally substituted with one or more substituents selected from methyl, methyl-d3, F or alkenyl; and
n is an integer from 0 to 4.
52. The compound of any one of embodiments 14-23 and 50-51, wherein moiety B is

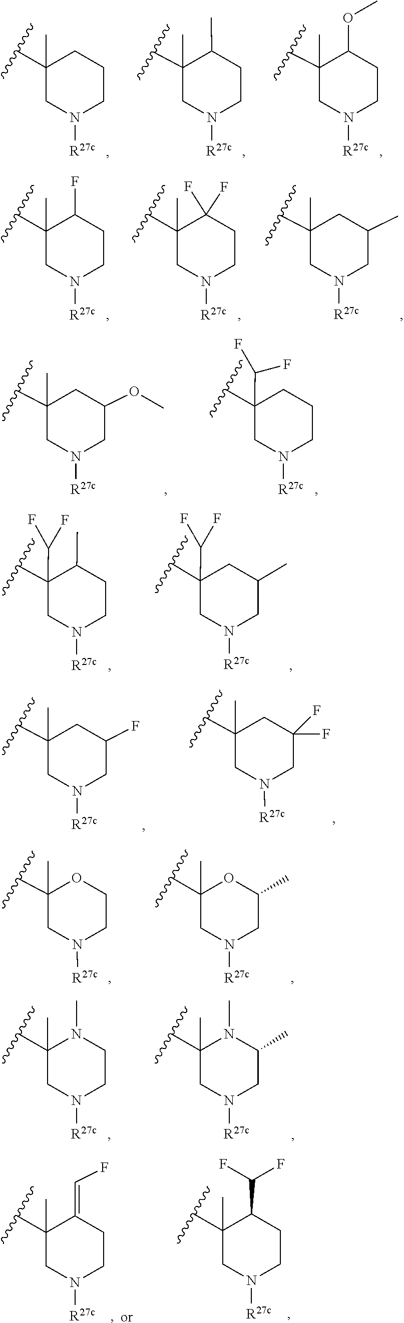

wherein R^{27c} is C₁₋₃alkyl, oxetanyl, tetrahydrofuranyl, or tetrahydropyranyl, wherein said C₁₋₃alkyl is optionally substituted with one or more substituents selected from deuterium, methyl, methyl-d3, F, cyclopropylmethyl, or alkenyl.
53. The compound of any one of embodiments 14-23 and 50-52, wherein moiety B is

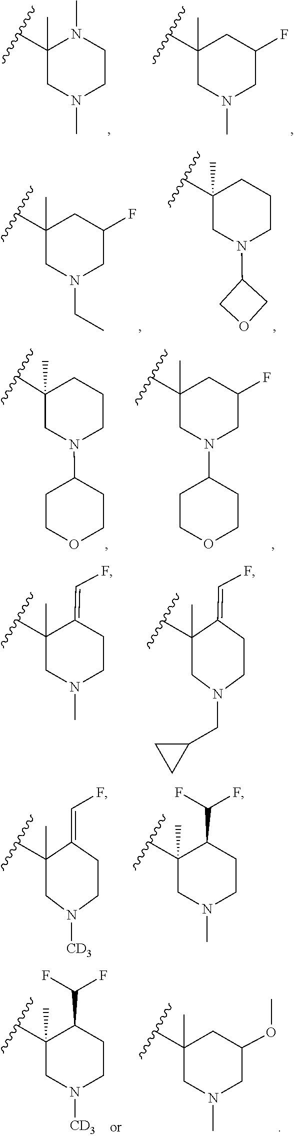

54. The compound of any one of embodiments 14-23, wherein moiety B is substituted or unsubstituted bicyclic heterocyclyl.
55. The compound of any one of embodiments 14-23, wherein moiety B is substituted or unsubstituted bicyclic heterocyclyl containing one or two heteroatoms selected from N or O, provided at least one of fusion atoms is N; preferably substituted or unsubstituted hexahydro-1H-pyrrolizinyl, substituted or unsubstituted octahydroindolizinyl, substituted or unsubstituted hexahydro-1H-pyrrolo[2,1-c][1,4]oxazinyl, or substituted or unsubstituted octahydropyrido[2,1-c][1,4]oxazinyl.
56. The compound of any one of embodiments 14-23 and 54-55, wherein moiety B is

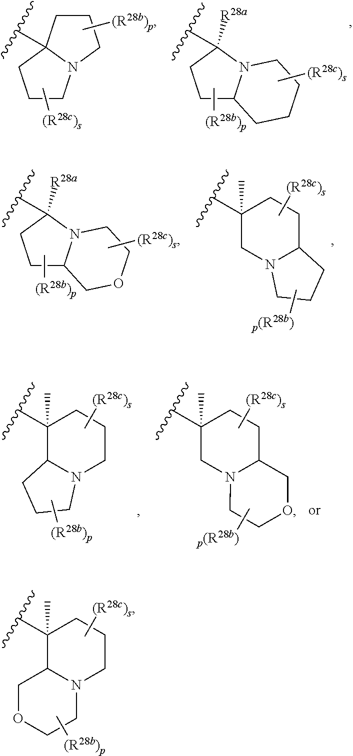

wherein
R^{28a} is H, methyl, methyl-d3, fluoromethyl, difluoromethyl, or trifluoromethyl;
each of R^{28b} and R^{28c} is H, halogen, substituted or unsubstituted alkyl, substituted or unsubstituted alkoxy, or one pair of R^{28b} or R^{28c} together with the atom(s) to which they are attached to, form unsubstituted or substituted alkenyl, or unsubstituted or substituted cycloalkyl; and
each of s and p is, independently, 0, 1, 2, 3, or 4;
provided

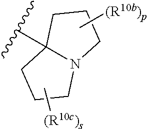

is not

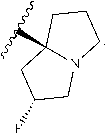 [SMILES: *[C@@]12CCCN1C[C@H](F)C2]

57. The compound of any one of embodiments 14-23 and 54-56, wherein moiety B is

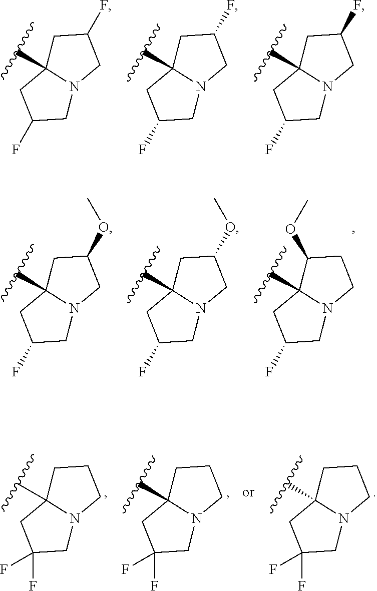

58. The compound of any one of embodiments 14-23 and 54-56, wherein moiety B is

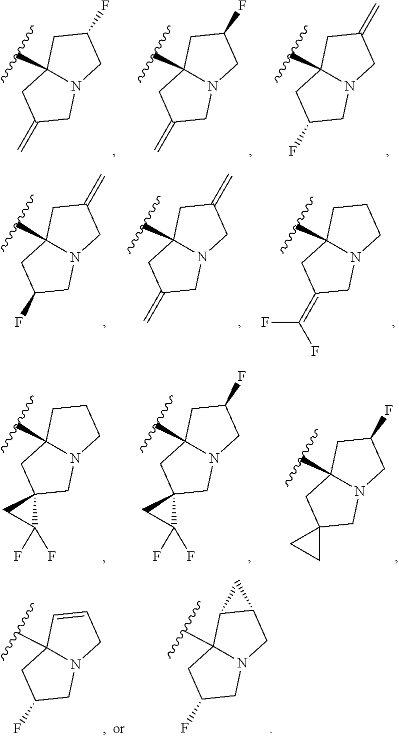

59. The compound of any one of embodiments 14-23, wherein moiety B is substituted or unsubstituted bicyclic heterocyclyl containing one or two heteroatoms selected from N or O, provided both fusion atoms are C; preferably substituted or unsubstituted octahydro-1H-cyclopenta[b]pyridinyl, substituted or unsubstituted octahydrocyclopenta[b][1,4]oxazinyl, or substituted or unsubstituted octahydrofuro[3,2-b]pyridinyl.
60. The compound of any one of embodiments 14-23 and 59, wherein moiety B is

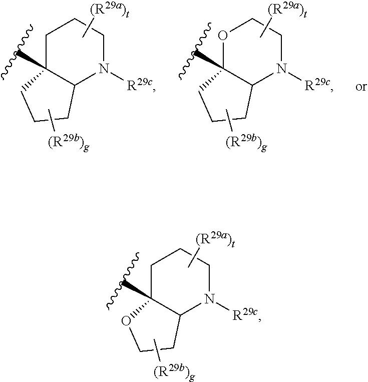

wherein
R^{29c} is methyl, methyl-d3, difluoromethyl, or trifluoromethyl;
each of R^{29a} and R^{29b} is H, halogen, substituted or unsubstituted alkyl; and
each of t and g is, independently, 0, 1, 2, 3, or 4.
61. The compound of any one of embodiments 14-23, wherein moiety B is substituted or unsubstituted cyclobutyl, substituted or unsubstituted cyclopentyl, substituted or unsubstituted cycloheptyl; preferably substituted or unsubstituted 2-dimethylamino-cyclobutyl, substituted or unsubstituted 2-dimethylamino-cyclopentyl, substituted or unsubstituted 2-dimethylamino-cycloheptyl; more preferably 2-dimethylamino-cyclobutyl, 2-dimethylamino-cyclopentyl, or 2-dimethylamino-cycloheptyl, wherein said each of 2-dimethylamino-cyclobutyl, 2-dimethylamino-cyclopentyl and 2-dimethylamino-cycloheptyl is optionally substituted with H, halogen, methoxy, or methoxy-d3.
62. The compound of any one of embodiments 14-23 and 61, wherein moiety B is

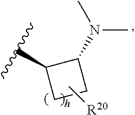

wherein
R²⁰ is H, halogen, methyl, methyl-d3, methoxy, or methoxy-d3; and
h is 1, 2, or 3.
63. The compound of any one of embodiments 14-23, wherein

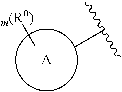

is

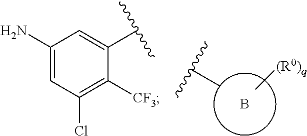

is

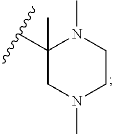 [SMILES: *C1(C)CN(C)CCN1C]

and all of R^{1a}, R^{1b}, R^{2a} and R^{2b} are H.
64. The compound of any one of embodiments 14-23, wherein

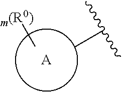

is

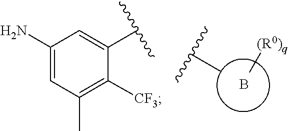

is

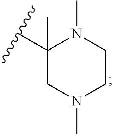 [SMILES: *C1(C)CN(C)CCN1C]

and all of R^{1a}, R^{1b}, R^{2a} and R^{2b} are H.
65. The compound of any one of embodiments 14-23, wherein

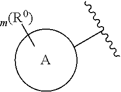

is

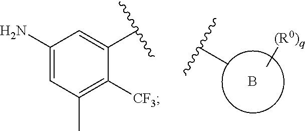

is

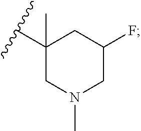 [SMILES: *C1(C)CC(F)CN(C)C1]

and all of R^{1a}, R^{1b}, R^{2a} and R^{2b} are H.
66. The compound of any one of embodiments 14-23, wherein

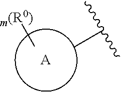

is

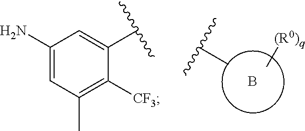

is

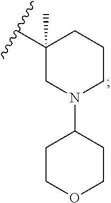 [SMILES: *[C@@]1(C)CCCN(C2CCOCC2)C1]

and all of R^{1a}, R^{1b}, R^{2a} and R^{2b} are H.
67. The compound of any one of embodiments 14-23, wherein

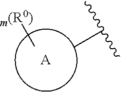

is

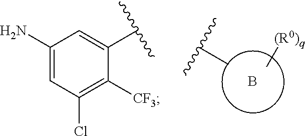

is

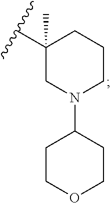 [SMILES: *[C@@]1(C)CCCN(C2CCOCC2)C1]

and all of R^{1a}, R^{1b}, R^{2a} and R^{2b} are H.

68. The compound of any one of embodiments 14-23, wherein

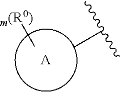

is

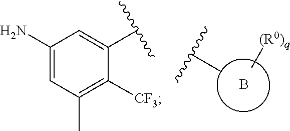

is

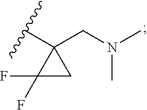 [SMILES: *C1(CN(C)C)CC1(F)F]

and all of R^{1a}, R^{1b}, R^{2a} and R^{2b} are H.

69. The compound of any one of embodiments 14-23, wherein

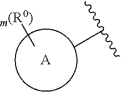

is

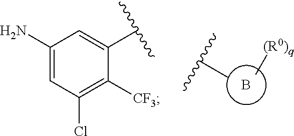

is

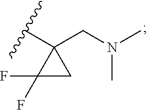 [SMILES: *C1(CN(C)C)CC1(F)F]

and all of R^{1a}, R^{1b}, R^{2a} and R^{2b} are H.
70. The compound of any one of embodiments 14-23, wherein

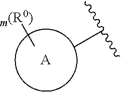

is

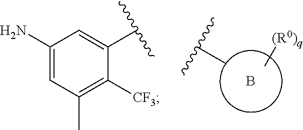

is

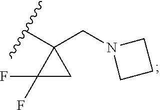 [SMILES: *C1(CN2CCC2)CC1(F)F]

and all of R^{1a}, R^{1b}, R^{2a} and R^{2b} are H.
71. The compound of any one of embodiments 14-23, wherein

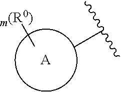

is

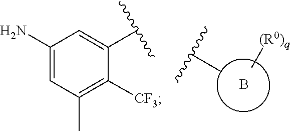

is

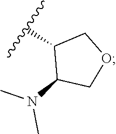 [SMILES: *[C@@H]1COC[C@H]1N(C)C]

and all of R^{1a}, R^{1b}, R^{2a} and R^{2b} are H.
72. The compound of any one of embodiments 14-23, wherein

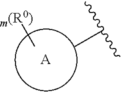

is

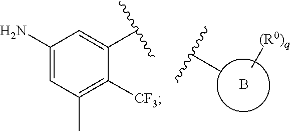

is

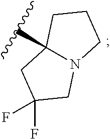 [SMILES: *[C@@]12CCCN1CC(F)(F)C2]

and all of R^{1a}, R^{1b}, R^{2a} and R^{2b} are H.
73. The compound of any one of embodiments 14-23, wherein

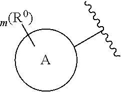

is

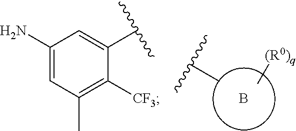

is

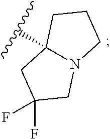 [SMILES: *[C@]12CCCN1CC(F)(F)C2]

and all of R^{1a}, R^{1b}, R^{2a} and R^{2b} are H.
74. The compound of any one of embodiments 14-23, wherein

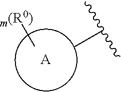

is

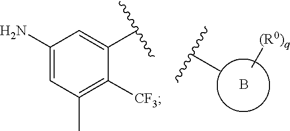

is

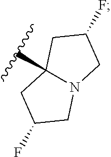 [SMILES: *[C@@]12C[C@H](F)CN1C[C@H](F)C2]

and all of R^{1a}, R^{1b}, R^{2a} and R^{2b} are H.
75. The compound of any one of embodiments 14-23, wherein

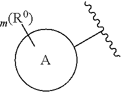

is

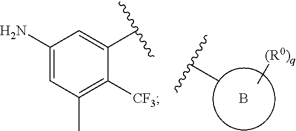

is

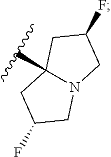

is F and all of R^{1a}, R^{1b}, R^{2a} and R^{2b} are H.
76. The compound of any one of embodiments 14-23, wherein

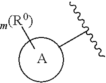

is

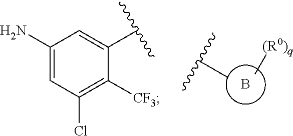

is

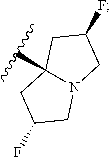

and all of R^{1a}, R^{1b}, R^{2a} and R^{2b} are H.
77. The compound of any one of embodiments 14-23, wherein

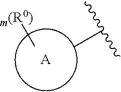

is

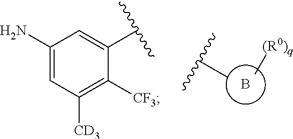

is

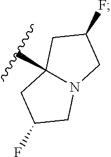

and all of R^{1a}, R^{1b}, R^{2a} and R^{2b} are H.
78. The compound of any one of embodiments 14-23, wherein

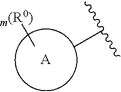

is

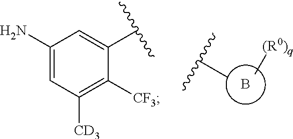

is

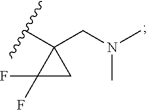 [SMILES: *C1(CN(C)C)CC1(F)F]

and all of R^{1a}, R^{1b}, R^{2a} and R^{2b} are H.
79. The compound of any one of embodiments 14-23, wherein

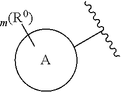

is

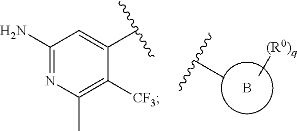

is

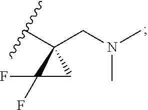 [SMILES: *[C@]1(CN(C)C)CC1(F)F]

and all of R^{1a}, R^{1b}, R^{2a} and R^{2b} are H.
80. The compound of any one of embodiments 14-23, wherein

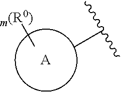

is

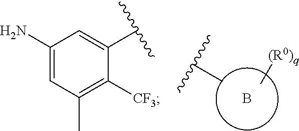

is

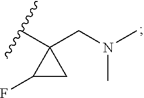 [SMILES: *C1(CN(C)C)CC1F]

and all of R^{1a}, R^{1b}, R^{2a} and R^{2b} are H.
81. The compound of any one of embodiments 14-23, wherein

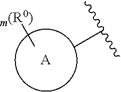

is

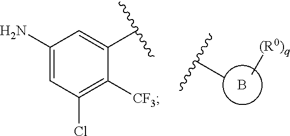

is

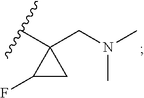 [SMILES: *C1(CN(C)C)CC1F]

and all of R^{1a}, R^{1b}, R^{2a} and R^{2b} are H.
82. The compound of any one of embodiments 14-23, wherein

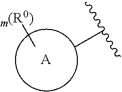

is

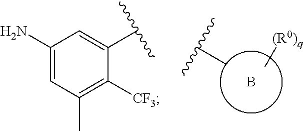

is

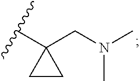 [SMILES: *C1(CN(C)C)CC1]

and all of R^{1a}, R^{1b}, R^{2a} and R^{2b} are H.
83. The compound of any one of embodiments 14-23, wherein

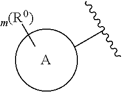

is

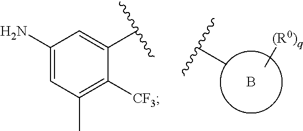

is

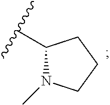 [SMILES: *[C@@H]1CCCN1C]

and all of R^{1a}, R^{1b}, R^{2a} and R^{2b} are H.
84. The compound of any one of embodiments 14-23, wherein

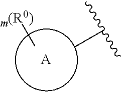

is

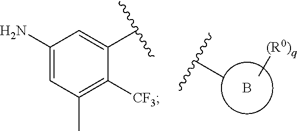

is

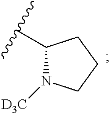 [SMILES: *[C@@H]1CCCN1C([2H])([2H])[2H]]

and all of R^{1a}, R^{1b}, R^{2a} and R^{2b} are H.
85. The compound of any one of embodiments 14-23, wherein

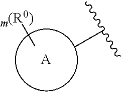

is

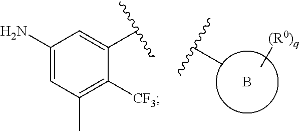

is

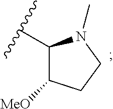 [SMILES: *[C@@H]1[C@@H](OC)CCN1C]

and all of R^{1a}, R^{1b}, R^{2a} and R^{2b} are H.
86. The compound of any one of embodiments 14-23, wherein

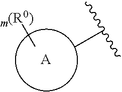

is

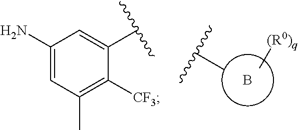

is

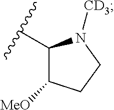 [SMILES: *[C@@H]1[C@@H](OC)CCN1C([2H])([2H])[2H]]

and all of R^{1a}, R^{1b}, R^{2a} and R^{2b} are H.
87. The compound of any one of embodiments 14-23, wherein

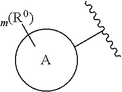

is

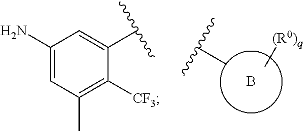

is

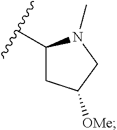 [SMILES: *[C@@H]1C[C@@H](OC)CN1C]

and all of R^{1a}, R^{1b}, R^{2a} and R^{2b} are H.
88. The compound of any one of embodiments 14-23, wherein

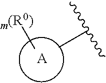

is

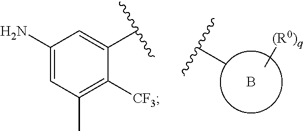

is

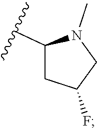 [SMILES: *[C@@H]1C[C@@H](F)CN1C]

and all of R^{1a}, R^{1b}, R^{2a} and R^{2b} are H.
89. The compound of any one of embodiments 14-23, wherein

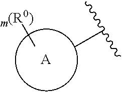

is

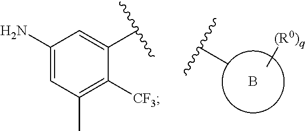

is

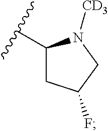 [SMILES: *[C@@H]1C[C@@H](F)CN1C([2H])([2H])[2H]]

and all of R^{1a}, R^{1b}, R^{2a} and R^{2b} are H.
90. The compound of any one of embodiments 14-23, wherein

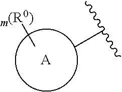

is

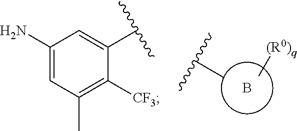

is

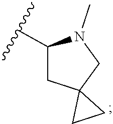 [SMILES: *[C@@H]1CC2(CC2)CN1C]

and all of R^{1a}, R^{1b}, R^{2a} and R^{2b} are H.
91. The compound of any one of embodiments 14-23, wherein

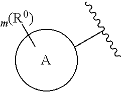

is

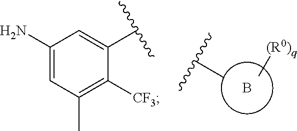

is

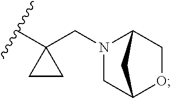 [SMILES: *C1(CN2C[C@@H]3C[C@H]2CO3)CC1]

and all of R^{1a}, R^{1b}, R^{2a} and R^{2b} are H.
92. The compound of any one of embodiments 14-23, wherein

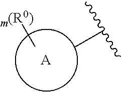

is

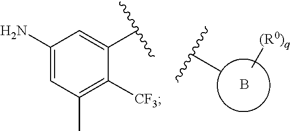

is

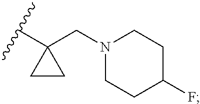 [SMILES: *C1(CN2CCC(F)CC2)CC1]

and all of R^{1a}, R^{1b}, R^{2a} and R^{2b} are H.
93. The compound of any one of embodiments 14-23, wherein

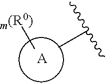

is

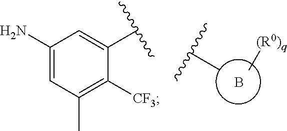

is

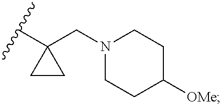 [SMILES: *C1(CN2CCC(OC)CC2)CC1]

and all of R^{1a}, R^{1b}, R^{2a} and R^{2b} are H.
94. The compound of any one of embodiments 14-23, wherein

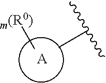

is

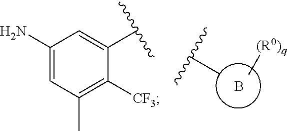

is

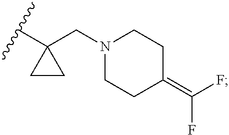 [SMILES: *C1(CN2CCC(=C(F)F)CC2)CC1]

and all of R^{1a}, R^{1b}, R^{2a} and R^{2b} are H.
95. The compound of any one of embodiments 14-23, wherein

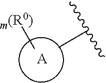

is

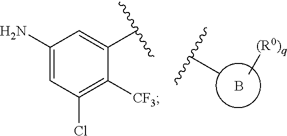

is

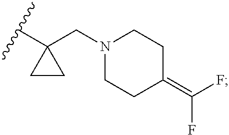 [SMILES: *C1(CN2CCC(=C(F)F)CC2)CC1]

and all of R^{1a}, R^{1b}, R^{2a} and R^{2b} are H.
96. The compound of any one of embodiments 14-23, wherein

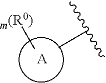

is

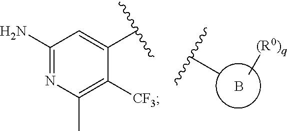

is

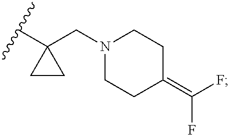 [SMILES: *C1(CN2CCC(=C(F)F)CC2)CC1]

and all of R^{1a}, R^{1b}, R^{2a} and R^{2b} are H.
97. The compound of any one of embodiments 14-23, wherein

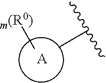

is

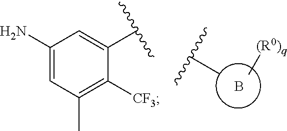

is

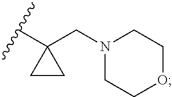 [SMILES: *C1(CN2CCOCC2)CC1]

and all of R^{1a}, R^{1b}, R^{2a} and R^{2b} are H.
98. The compound of any one of embodiments 14-23, wherein

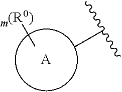

is

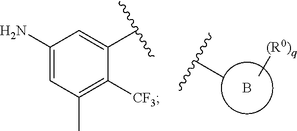

is

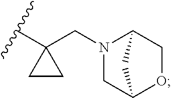 [SMILES: *C1(CN2C[C@H]3C[C@@H]2CO3)CC1]

and all of R^{1a}, R^{1b}, R^{2a} and R^{2b} are H.
99. The compound of any one of embodiments 14-23, wherein

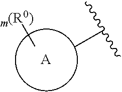

is

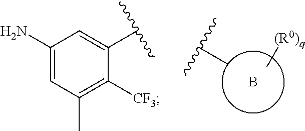

is

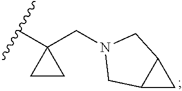 [SMILES: *C1(CN2CC3CC3C2)CC1]

and all of R^{1a}, R^{1b}, R^{2a} and R^{2b} are H.
100. The compound of any one of embodiments 14-23, wherein

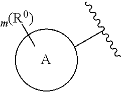

is

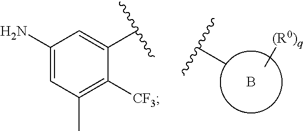

is

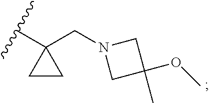 [SMILES: *C1(CN2CC(C)(OC)C2)CC1]

and all of R^{1a}, R^{1b}, R^{2a} and R^{2b} are H.
101. The compound of any one of embodiments 14-23, wherein

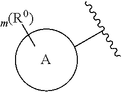

is

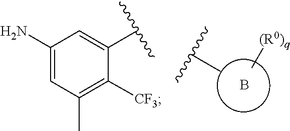

is

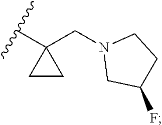 [SMILES: *C1(CN2CC[C@@H](F)C2)CC1]

and all of R^{1a}, R^{1b}, R^{2a} and R² are H.
102. The compound of any one of embodiments 14-23, wherein

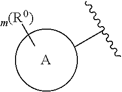

is

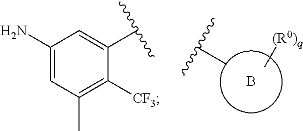

is

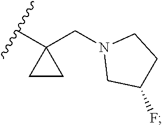 [SMILES: *C1(CN2CC[C@H](F)C2)CC1]

and all of R^{1a}, R^{1b}, R^{2a} and R^{2b} are H.
103. The compound of any one of embodiments 14-23, wherein

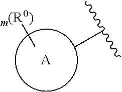

is

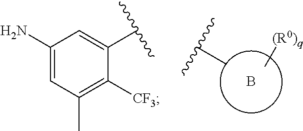

is

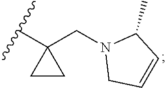 [SMILES: *C1(CN2CC=C[C@H]2C)CC1]

and all of R^{1a}, R^{1b}, R^{2a} and R^{2b} are H.
104. The compound of any one of embodiments 14-23, wherein

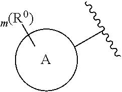

is

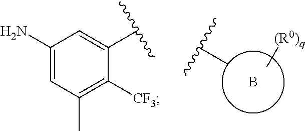

is

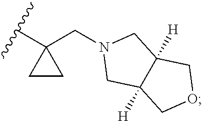 [SMILES: *C1(CN2C[C@H]3COC[C@H]3C2)CC1]

and all of R^{1a}, R^{1b}, R^{2a} and R^{2b} are H.
105. The compound of any one of embodiments 14-23, wherein

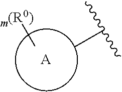

is

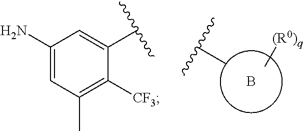

is

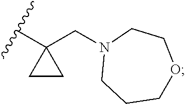 [SMILES: *C1(CN2CCCOCC2)CC1]

and all of R^{1a}, R^{1b}, R^{2a} and R^{2b} are H.
106. The compound of any one of embodiments 14-23, wherein

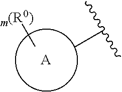

is

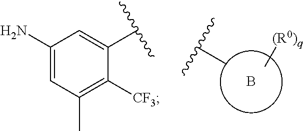

is

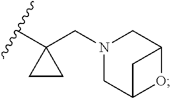 [SMILES: *C1(CN2CC3CC(C2)O3)CC1]

and all of R^{1a}, R^{1b}, R^{2a} and R^{2b} are H.
107. The compound of any one of embodiments 14-23, wherein

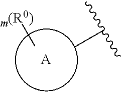

is

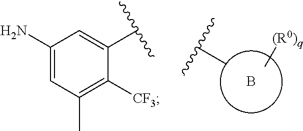

is

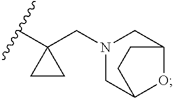 [SMILES: *C1(CN2CC3CCC(C2)O3)CC1]

and all of R^{1a}, R^{1b}, R^{2a} and R^{2b} are H.
108. The compound of any one of embodiments 14-23, wherein

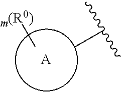

is

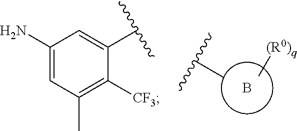

is

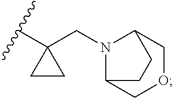 [SMILES: *C1(CN2C3CCC2COC3)CC1]

and all of R^{1a}, R^{1b}, R^{2a} and R^{2b} are H.
109. The compound of any one of embodiments 14-23, wherein

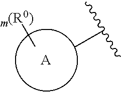

is

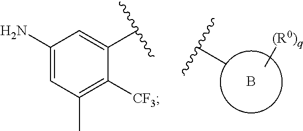

is

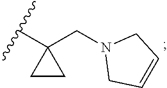 [SMILES: *C1(CN2CC=CC2)CC1]

and all of R^{1a}, R^{1b}, R^{2a} and R^{2b} are H.
110. The compound of any one of embodiments 14-23, wherein

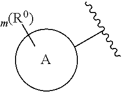

is

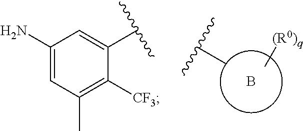

is

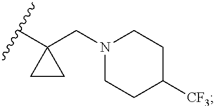 [SMILES: *C1(CN2CCC(C(F)(F)F)CC2)CC1]

and all ofR^{1a}, R^{1b}, R^{2a} and R² are H.
111. The compound of any one of embodiments 14-23, wherein

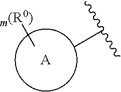

is

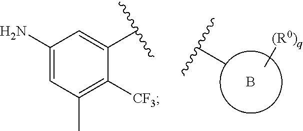

is

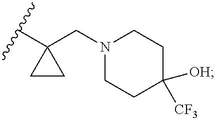 [SMILES: *C1(CN2CCC(O)(C(F)(F)F)CC2)CC1]

and all of R^{1a}, R^{1b}, R^{2a} and R^{2b} are H.
112. The compound of any one of embodiments 14-23, wherein

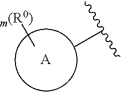

is

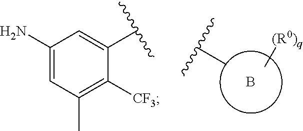

is

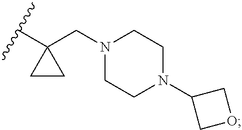 [SMILES: *C1(CN2CCN(C3COC3)CC2)CC1]

and all of R^{1a}, R^{1b}, R^{2a} and R^{2b} are H.
113. The compound of any one of embodiments 14-23, wherein

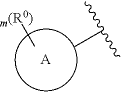

is

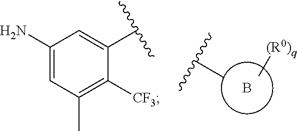

is

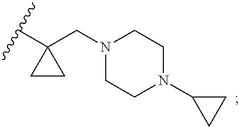 [SMILES: *C1(CN2CCN(C3CC3)CC2)CC1]

and all of R^{1a}, R^{1b}, R^{2a} and R^{2b} are H.
114. The compound of any one of embodiments 14-23, wherein

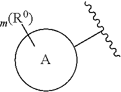

is

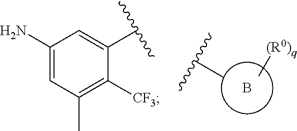

is

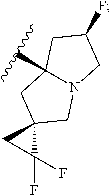 [SMILES: *[C@@]12C[C@@H](F)CN1C[C@@]1(CC1(F)F)C2]

and all of R^{1a}, R^{1b}, R^{2a} and R^{2b} are H.
115. The compound of any one of embodiments 14-23, wherein

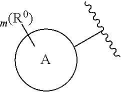

is

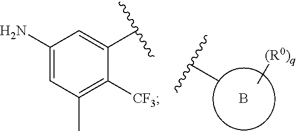

is

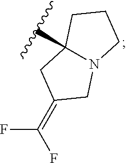 [SMILES: *[C@@]12CCCN1CC(=C(F)F)C2]

and all of R^{1a}, R^{1b}, R^{2a} and R^{2b} are H.
116. The compound of any one of embodiments 14-23, wherein

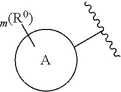

is

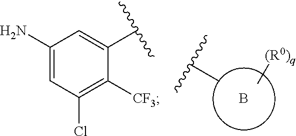

is

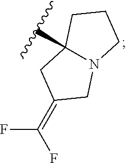 [SMILES: *[C@@]12CCCN1CC(=C(F)F)C2]

and all of R^{1a}, R^{1b}, R^{2a} and R^{2b} are H.
117. The compound of any one of embodiments 14-23, wherein

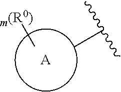

is

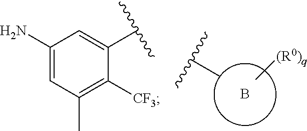

is

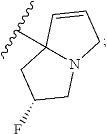 [SMILES: *C12C=CCN1C[C@H](F)C2]

and all of R^{1a}, R^{1b}, R^{2a} and R^{2b} are H.
118. The compound of any one of embodiments 14-23, wherein

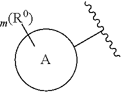

is

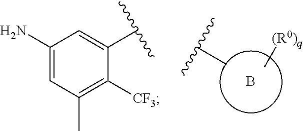

is

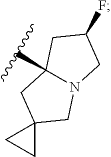 [SMILES: *[C@@]12C[C@@H](F)CN1CC1(CC1)C2]

and all of R^{1a}, R^{1b}, R^{2a} and R^{2b} are H.
119. The compound of any one of embodiments 14-23, wherein

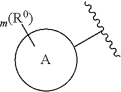

is

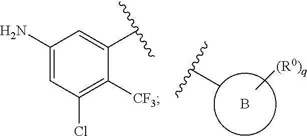

is

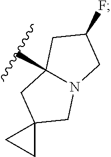 [SMILES: *[C@@]12C[C@@H](F)CN1CC1(CC1)C2]

and all of R^{1a}, R^{1b}, R^{2a} and R^{2b} are H.
120. The compound of any one of embodiments 14-23, wherein

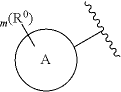

is

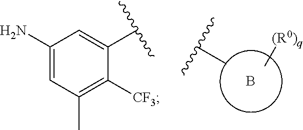

is

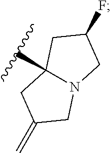 [SMILES: *[C@]12CC(=C)CN1C[C@H](F)C2]

and all of R^{1a}, R^{1b}, R^{2a} and R^{2b} are H.
121. The compound of any one of embodiments 14-23, wherein

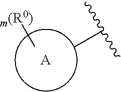

is

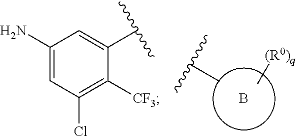

is

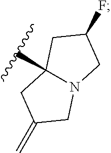 [SMILES: *[C@]12CC(=C)CN1C[C@H](F)C2]

and all of R^{1a}, R^{1b}, R^{2a} and R^{2b} are H.
122. The compound of any one of embodiments 14-23, wherein

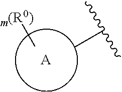

is

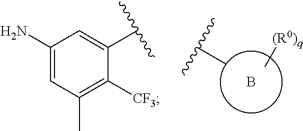

is

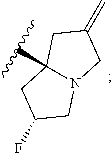 [SMILES: *[C@@]12CC(=C)CN1C[C@H](F)C2]

and all of R^{1a}, R^{1b}, R^{2a} and R^{2b} are H.
123. The compound of any one of embodiments 14-23, wherein

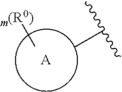

is

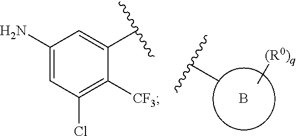

is

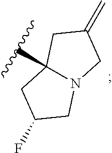 [SMILES: *[C@@]12CC(=C)CN1C[C@H](F)C2]

and all of R^{1a}, R^{1b}, R^{2a} and R^{2b} are H.
124. The compound of any one of embodiments 14-23, wherein

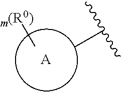

is

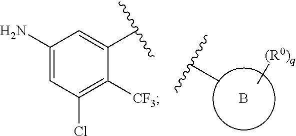

is

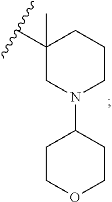 [SMILES: *C1(C)CCCN(C2CCOCC2)C1]

and all of R^{1a}, R^{1b}, R^{2a} and R^{2b} are H.
125. The compound of any one of embodiments 14-23, wherein

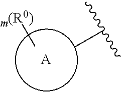

is

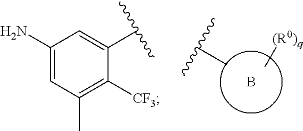

is

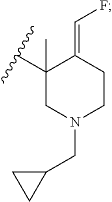 [SMILES: *C1(C)CN(CC2CC2)CC/C1=C\F]

and all of R^{1a}, R^{1b}, R^{2a} and R^{2b} are H.
126. The compound of any one of embodiments 14-23, wherein

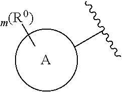

is

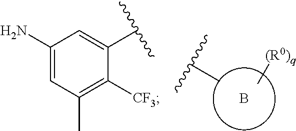

is

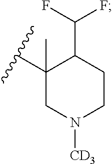 [SMILES: *C1(C)CN(C([2H])([2H])[2H])CCC1C(F)F]

all of R^{1a}, R^{1b}, R^{2a} and R^{2b} are H; and both R^{3a} and R^{3b} are H, wherein the H refers to deuterium.
127. The compound of any one of embodiments 14-23, wherein

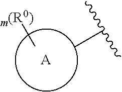

is

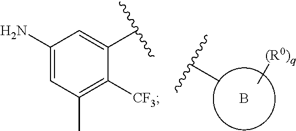

is

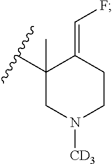 [SMILES: *C1(C)CN(C([2H])([2H])[2H])CC/C1=C\F]

all of R^{1a}, R^{1b}, R^{2a} and R^{2b} are H; and both R^{3a} and R^{3b} are H, wherein the H refers to deuterium.
128. The compound of any one of embodiments 14-23, wherein

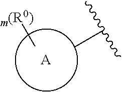

is

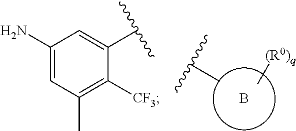

is

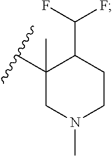 [SMILES: *C1(C)CN(C)CCC1C(F)F]

and all of R^{1a}, R^{1b}, R^{2a} and R^{2b} are H.
129. The compound of any one of embodiments 14-23, wherein

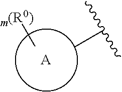

is

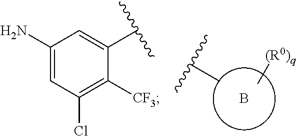

is

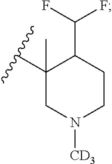 [SMILES: *C1(C)CN(C([2H])([2H])[2H])CCC1C(F)F]

all of R^{1a}, R^{1b}, R^{2a} and R^{2b} are H; and both R^{3a} and R^{3b} are H, wherein the H refers to deuterium.
130. The compound of any one of embodiments 14-23, wherein

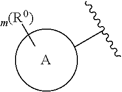

is

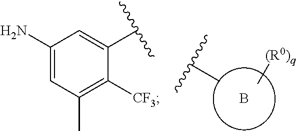

is

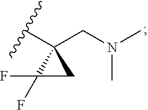 [SMILES: *[C@@]1(CN(C)C)CC1(F)F]

R^{1a} is methyl; and all of R^{1b}, R^{2a} and R^{2b} are H.
131. The compound of embodiment 1, wherein L¹ is a direct bond.
132. The compound of any one of embodiments 1 or 131, wherein moiety B is substituted or unsubstituted azetidinyl, substituted or unsubstituted diazaspiro[3.3]heptanyl, substituted or unsubstituted diazaspiro[3.4]octanyl, or substituted or unsubstituted diazaspiro[3.5]nonanyl; preferably substituted or unsubstituted 1,6-diazaspiro[3.3]heptan-6-yl, substituted or unsubstituted 2,5-diazaspiro[3.4]octan-2-yl, or substituted or unsubstituted 2,5-diazaspiro[3.5]nonan-2-yl.
133. The compound of any one of embodiments 1 and 131-132 wherein moiety B is

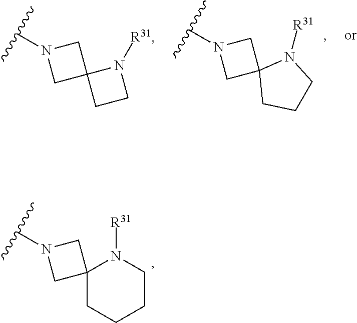

wherein R³¹ is H, substituted or unsubstituted C₁₋₄ alkyl.
134. The compound of embodiment 133, wherein R³¹ is H, methyl, methyl-d3, or ethyl.
135. The compound of any one of embodiments 1 or 131, wherein the compound of formula (I) is a compound of formula (IIc):

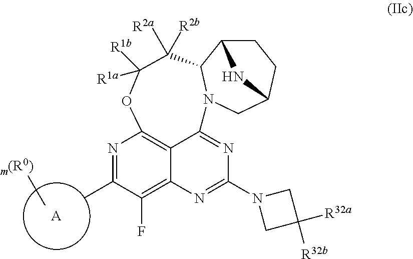

wherein
each of R^{32a} and R^{32b}, is, independently, H, unsubstituted or substituted C₁₋₄alkyl, unsubstituted or substituted amino, or unsubstituted or substituted heterocyclyl.
136. The compound of embodiment 135, wherein R^{32a} is dimethylamino, ethyl(methyl)amino, diethylamino, azetidine-1-yl, 3-fluoroazetidin-1-yl, 3-methoxyazetidin-1-yl, pyrrolidin-1-yl, 2-fluorrpyrrolidin-1-yl, 2-methoxypyrrolidin-1-yl, piperidin-1-yl, 2-fluoropiperidin-1-yl, 3-fluoropiperidin-1-yl, 2-methoxypiperidin-1-yl, 3-methoxypiperidin-1-yl, morpholino, 3-oxa-6-azabicyclo[3.1.1]heptan-6-yl, 6-oxa-3-azabicyclo[3.1.1]heptan-3-yl, (1S,4S)-2-oxa-5-azabicyclo[2.2.1]heptan-5-yl, (1R,4R)-2-oxa-5-azabicyclo[2.2.1]heptan-5-yl, 3-oxa-8-azabicyclo[3.2.1]octan-8-yl, 8-azabicyclo[3.2.1]octan-8-yl, 8-oxa-3-azabicyclo[3.2.1]octan-3-yl, 3-azabicyclo[3.1.1]heptan-3-yl, 2-oxa-6-azaspiro[3.3]heptan-6-yl, or 6-azabicyclo[3.1.1]heptan-6-yl.
137. The compound of any one of embodiments 135-136, wherein R^{32b} is H, methyl, methyl-d3, fluoromethyl, methoxymethyl, or ethyl; preferably H, methyl, or methyl-d3.
138. The compound of any one of embodiments 135-137, wherein the

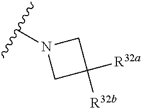

is

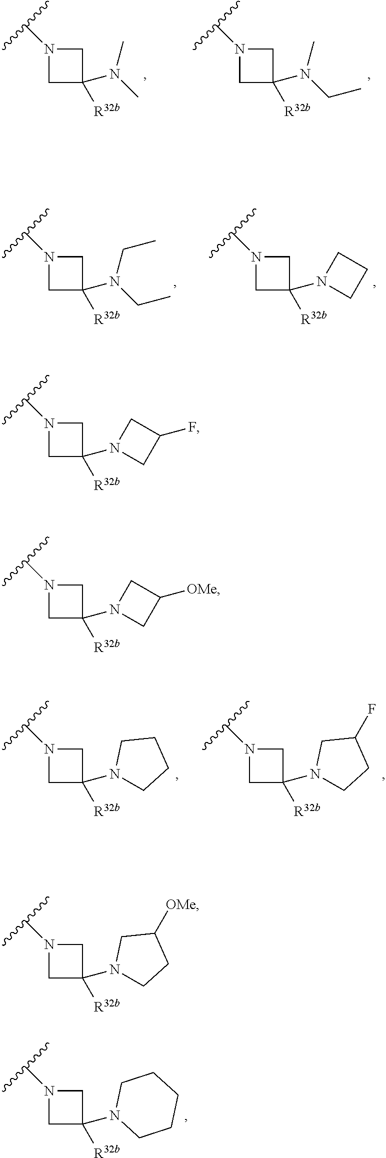

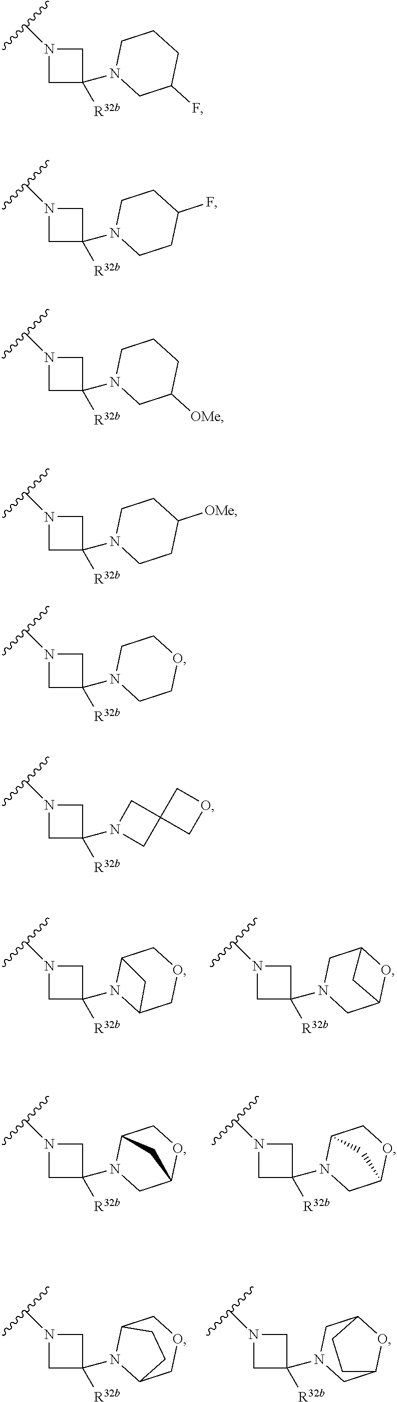

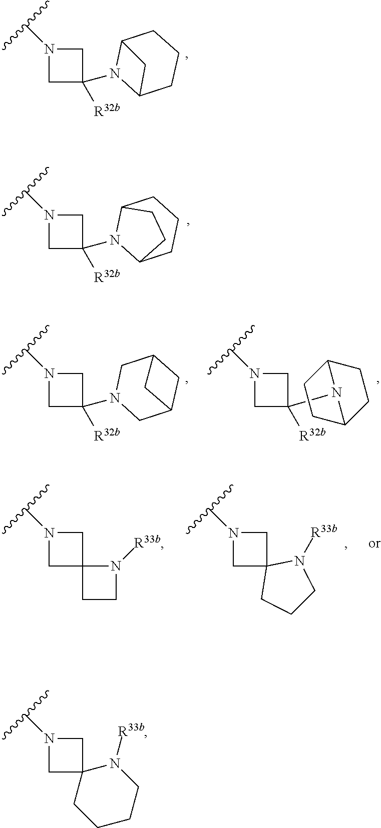

wherein R^{32b} is H, methyl, methyl-d3, fluoromethyl, methoxymethyl, or ethyl; and
R^{33b} is H, methyl, methyl-d3 or ethyl.
139. The compound of any one of embodiments 1-12, 14-58 and 131-138, wherein each of R^{1a}, R^{1b}, R^{2a} and R^{2b}, is, independently, H, or methyl, or R^{1a} and R^{1b}, together form an oxo or a substituted or unsubstituted cyclopropyl.
140. The compound of any one of embodiments 1-12, 14-58 and 131-139, wherein all of R^{1a}, R^{1b}, R^{2a} and R^{2b} are H.
141. The compound of any one of embodiments 1-12, 14-58 and 131-140, wherein ring A is substituted or unsubstituted phenyl, or substituted or unsubstituted naphthyl; preferably ring A is phenyl or naphthyl, wherein said phenyl and naphthyl are optionally substituted with 1, 2, 3 or 4 substituents selected from amino, —Cl, —F, ethenyl, ethyl, methyl, trifluoromethyl, cyclopropyl, hydroxy, or 1,1-difluoroethyl.
142. The compound of any one of embodiments 1-12, 14-58 and 131-141, wherein ring A is phenyl optionally substituted with 1, 2, 3 or 4 substituents independently selected from amino, hydroxyl, —Cl, —F, ethyl, methyl, methyl-d3, trifluoromethyl, or cyclopropyl; preferably aminophenyl optionally substituted with 1, 2, 3 or 4 substituents independently selected from —Cl, —F, ethyl, methyl, methyl-d3, trifluoromethyl, or cyclopropyl.
143. The compound of any one of embodiments 1-12, 14-58 and 131-142, wherein

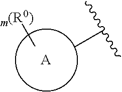

is

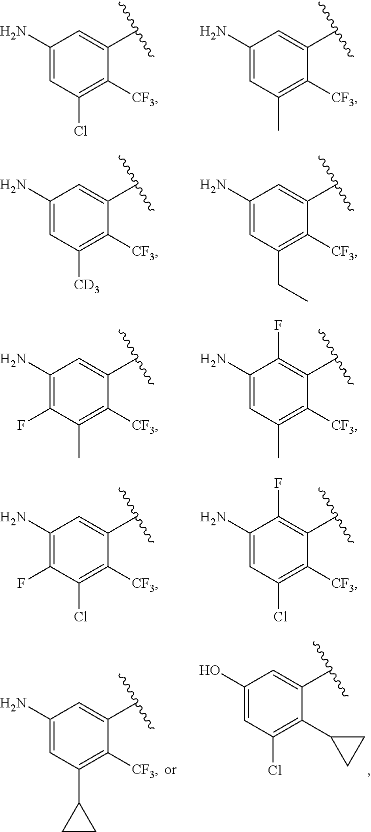

144. The compound of any one of embodiments 1-12, 14-58 and 131-141, wherein ring A is naphthyl optionally substituted with 1, 2, 3 or 4 substituents independently selected from —Cl, —F, ethenyl, ethyl, cyclopropyl, or hydroxy; preferably hydroxylnaphthyl optionally substituted with 1, 2, 3 or 4 substituents independently selected from —Cl, —F, ethenyl, ethyl, or cyclopropyl.
145. The compound of any one of embodiments 1-12, 14-58, 131-141 and 144, wherein

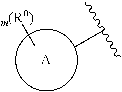

is

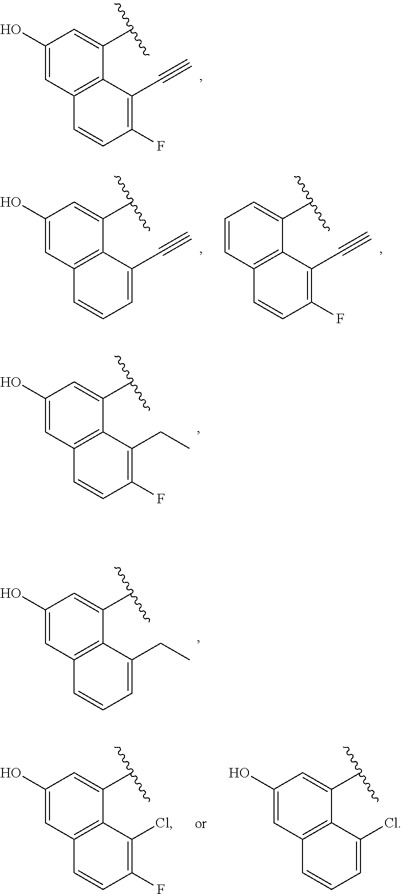

146. The compound of any one of embodiments 1-12, 14-58 and 131-141, wherein

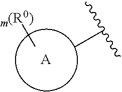

is

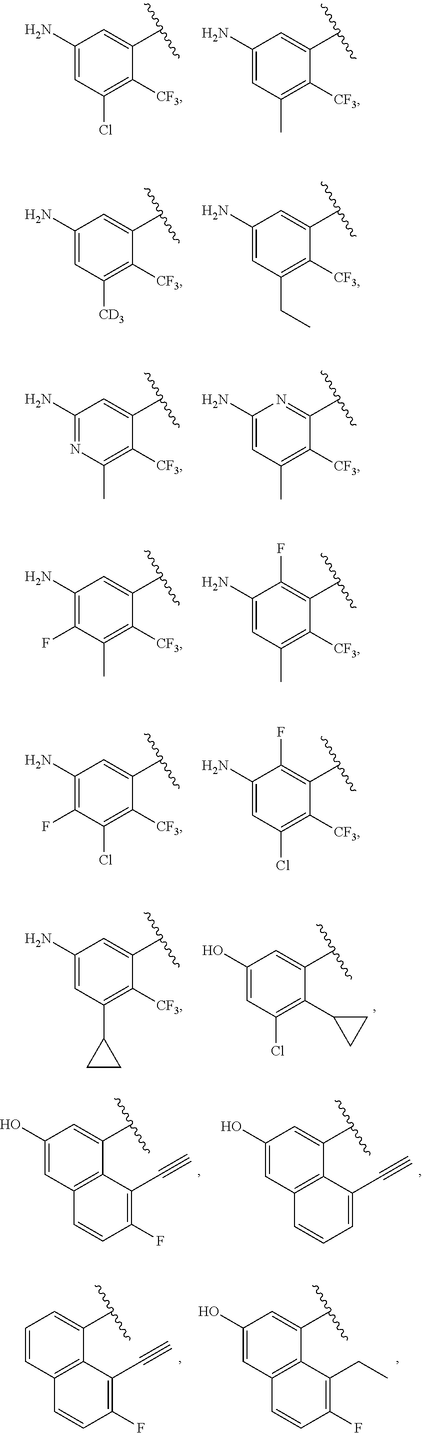

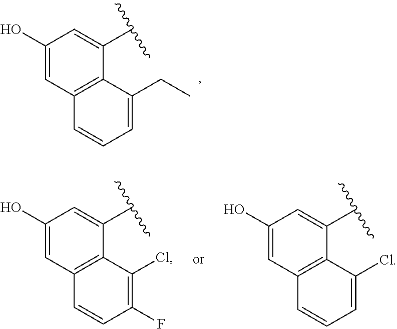 [SMILES: *c1cc(O)cc2cccc(CC)c12 ; *c1cc(O)cc2ccc(F)c(Cl)c12 ; *c1cc(O)cc2cccc(Cl)c12]

147. The compound of embodiment 1, the compound is a compound of formula (IVb):

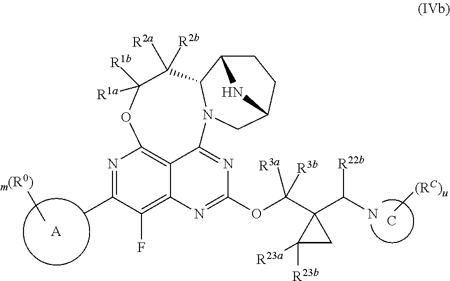

wherein
ring C is is substituted or unsubstituted 4- to 7-membered heterocyclyl optionally containing one or more additional heteroatom selected from N or O;
each of R^{c} is, independently, H, halogen, amino, —OH, —CN, unsubstituted or substituted C₁₋₄alkyl, or unsubstituted or substituted C₁₋₄alkoxy, or one pair of the R^{c} groups, together with the atom(s) to which they are attached to, form unsubstituted or substituted bridge, unsubstituted or substituted cycloalkyl, or unsubstituted or substituted heterocyclyl.
u is an integer from 0 to the maximum number of the substituent groups allowed on rings C; and
the other variables are defined above.
148. The compound of embodiment 1, the compound is a compound of formula (Vb):

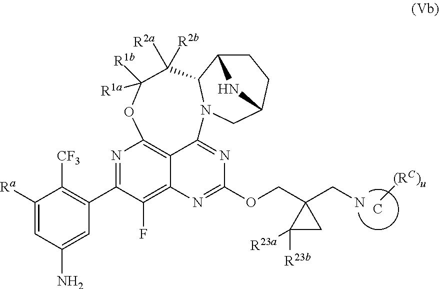

wherein
R^{a} is methyl, methyl-d3, or Cl;
ring C is is substituted or unsubstituted 4- to 7-membered heterocyclyl optionally containing one or more additional heteroatom selected from N or 0;
each of R^{c} is, independently, H, halogen, amino, —OH, —CN, unsubstituted or substituted C₁₋₄alkyl, or unsubstituted or substituted C₁₋₄alkoxy, or one pair of the R^{c} groups, together with the atom(s) to which they are attached to, form unsubstituted or substituted bridge, unsubstituted or substituted cycloalkyl, or unsubstituted or substituted heterocyclyl.
u is an integer from 0 to the maximum number of the substituent groups allowed on rings C; and
the other variables are defined above.
149. The compound of embodiment 1, the compound is a compound of formula (IVc):

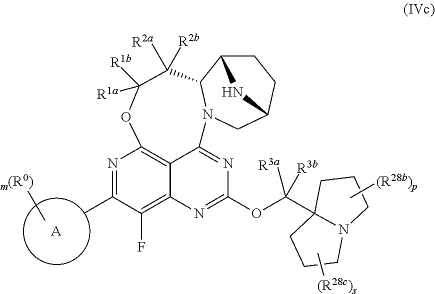

wherein
each of R^{28b} and R^{28c} is H, halogen, substituted or unsubstituted alkyl, substituted or unsubstituted alkoxy, or one pair of R^{28b} or R^{28c} together with the atom(s) to which they are attached to, form unsubstituted or substituted alkenyl, or unsubstituted or substituted cycloalkyl; and
each of s and p is, independently, 0, 1, 2, 3, or 4;
provided

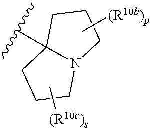

is not

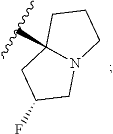 [SMILES: *[C@@]12CCCN1C[C@H](F)C2]

and
the other variables are defined above.
150. The compound of embodiment 1, the compound is a compound of formula (Vc):

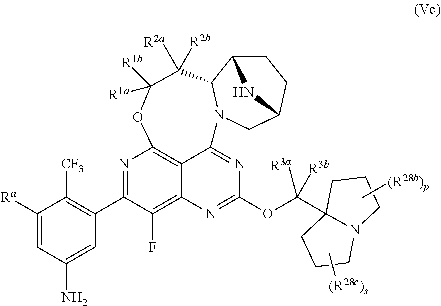

wherein
R^{a} is methyl, methyl-d3, or Cl;
each of R^{28b} and R^{28c} is H, halogen, substituted or unsubstituted alkyl, substituted or unsubstituted alkoxy, or one pair of R^{28b} or R^{28c} together with the atom(s) to which they are attached to, form unsubstituted or substituted alkenyl, or unsubstituted or substituted cycloalkyl; and
each of s and p is, independently, 0, 1, 2, 3, or 4;
provided

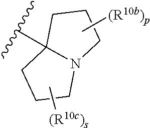

is not

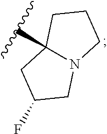 [SMILES: *[C@@]12CCCN1C[C@H](F)C2]

and
the other variables are defined above.
151. The compound of any one of embodiments 1-150, wherein the compound is one of the compounds in Table 2.
152. A pharmaceutical composition comprising a compound of any one of embodiments 1-151, or a pharmaceutically acceptable salt, tautomer, stereoisomer, enantiomer, or atropisomer thereof, and a pharmaceutically acceptable carrier, excipient or vehicle.
153. A method for inhibiting the activity of KRAS mutant protein in a cell, comprising contacting said cell with a compound of any one of embodiments 1-151, or a pharmaceutically acceptable salt, tautomer, stereoisomer, enantiomer, or atropisomer thereof, optionally wherein the KRAS mutant protein is KRAS G12D mutant protein.
154. A method for treatment or prevention of cancer, the method comprising administering to a subject in need thereof a compound of any one of embodiments 1-151, or a pharmaceutically acceptable salt, tautomer, stereoisomer, enantiomer, or atropisomer thereof, optionally wherein the cancer is mediated by KRAS mutation; preferably KRAS G12D mutation.

The present embodiments can be understood more fully by reference to the detailed description and examples, which are intended to exemplify non-limiting embodiments.

### Methods for Making Compounds

The Compounds can be made using conventional organic syntheses and commercially available starting materials. By way of example and not limitation, Compounds of formula (I) can be prepared as outlined in Schemes 1-3 shown below as well as in the examples set forth herein. It should be noted that one skilled in the art would know how to modify the procedures set forth in the illustrative schemes and examples to arrive at the desired products. Common protecting groups may be used to prevent certain functional groups from undergoing undesired reaction. Exemplary protecting groups are described in “Protective Groups in Organic Synthesis”, 4^{th} Edition, P. G. M. Wuts; T. W. Greene, John Wiley, 2007, and references cited therein.

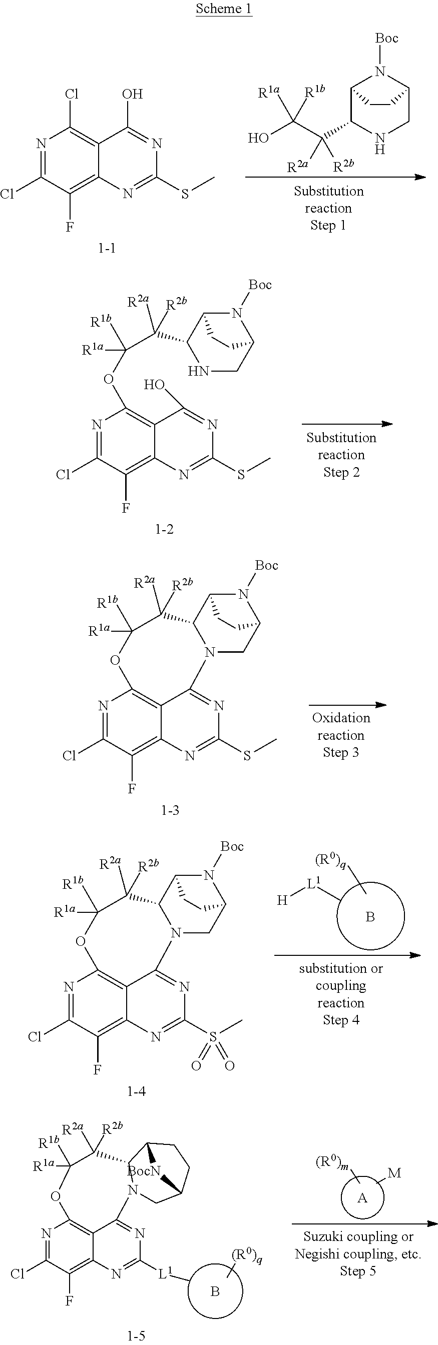

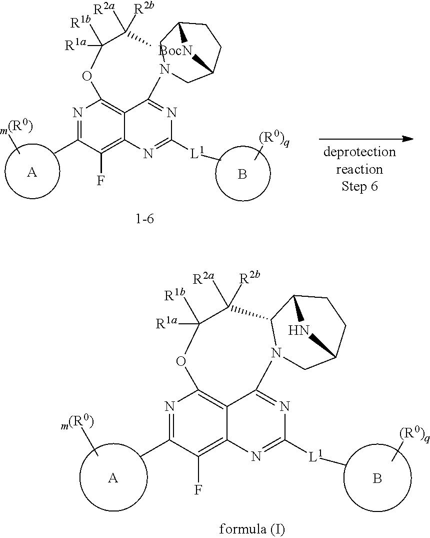

As shown in Scheme 1, in some embodiments, provided herein are methods for preparing the compounds defined as formula (I). Compound 1-1 is converted into compound 1-2 under substitution conditions (e.g., NaH, THF); then compound 1-2 is converted to compound 1-3 under substitution conditions (e.g., BOPCI, DIEA); then compound 1-3 is converted to compound 1-4 under oxidation conditions (e.g., NaIO₄, RuCl₃); then compound 1-4 is converted to compound 1-5 followed by substitution or coupling reactions (e.g., LIHMDS); compound 1-5 further undergoes metal catalyzed cross-coupling reaction such as Suzuki, Negishi, or Stille coupling (e.g. Pd(dtbpf)Cl₂, K₃PO₄, 1,4-dioxane, water for Suzuki coupling) to obtain compound 1-6, wherein M can be boronic acid, boronic ester, a metal (such as Zn), tributyltin, etc.; finally, compound 1-6 containing protection group is then deprotected (e.g., HCl/dioxane to deprotect Boc group) to yield the compound defined as formula (I).

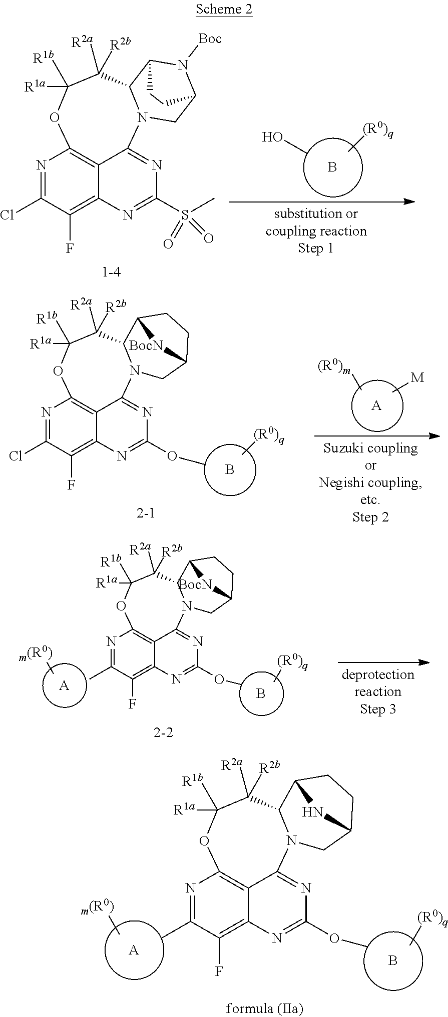

As shown in Scheme 2, in some embodiments, provided herein are methods for preparing the compounds defined as formula (IIa). Compound 1-4 is converted to compound 2-1 followed by substitution or coupling reactions (e.g., LIHMDS); compound 2-1 further undergoes metal catalyzed cross-coupling reaction such as Suzuki, Negishi, or Stille coupling (e.g. Pd(dtbpf)Cl₂, K₃PO₄, 1,4-dioxane, water for Suzuki coupling) to obtain compound 2-2, wherein M can be boronic acid, boronic ester, a metal (such as Zn), tributyltin, etc.; finally, compound 2-2 containing protection group is then deprotected (e.g., HCl/dioxane to deprotect Boc group) to yield the compound defined as formula (IIa).

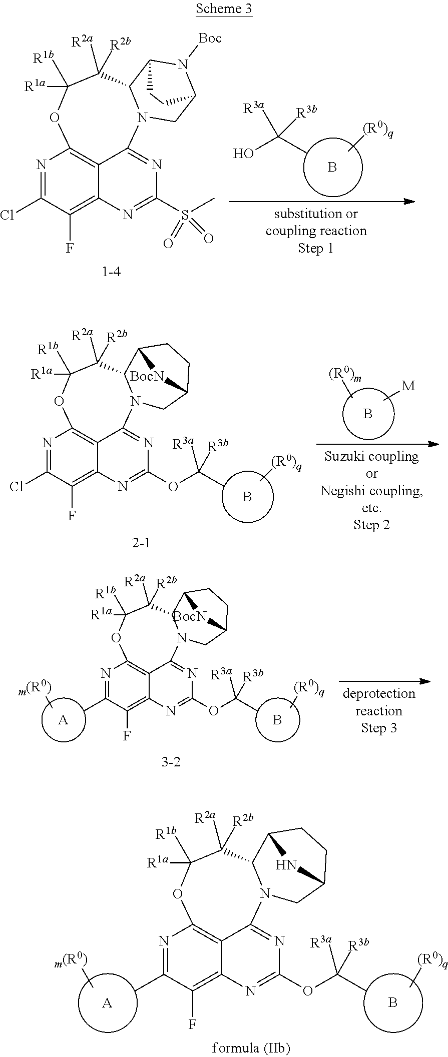

As shown in Scheme 3, in some embodiments, provided herein are methods for preparing the compounds defined as formula (IIb). Compound 1-4 is converted to compound 3-1 followed by substitution or coupling reactions (e.g., LIHMDS); compound 3-1 further undergoes metal catalyzed cross-coupling reaction such as Suzuki, Negishi, or Stille coupling (e.g. Pd(dtbpf)Cl₂, K₃PO₄, 1,4-dioxane, water for Suzuki coupling) to obtain compound 3-2, wherein M can be boronic acid, boronic ester, a metal (such as Zn), tributyltin, etc.; finally, compound 3-2 containing protection group is then deprotected (e.g., HCl/dioxane to deprotect Boc group) to yield the compound defined as formula (IIb).

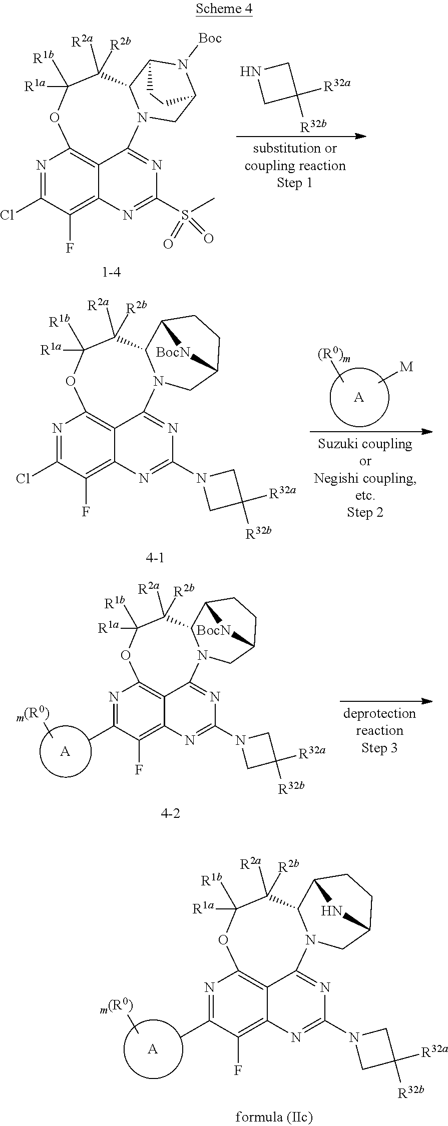

As shown in Scheme 4, in some embodiments, provided herein are methods for preparing the compounds defined as formula (IIc). Compound 1-4 is converted to compound 4-1 followed by substitution or coupling reactions (e.g., LIHMDS); compound 4-1 further undergoes metal catalyzed cross-coupling reaction such as Suzuki, Negishi, or Stille coupling (e.g. Pd(dtbpf)Cl₂, K₃PO₄, 1,4-dioxane, water for Suzuki coupling) to obtain compound 4-2, wherein M can be boronic acid, boronic ester, a metal (such as Zn), tributyltin, etc.; finally, compound 4-2 containing protection group is then deprotected (e.g., HCl/dioxane to deprotect Boc group) to yield the compound defined as formula (IIc).

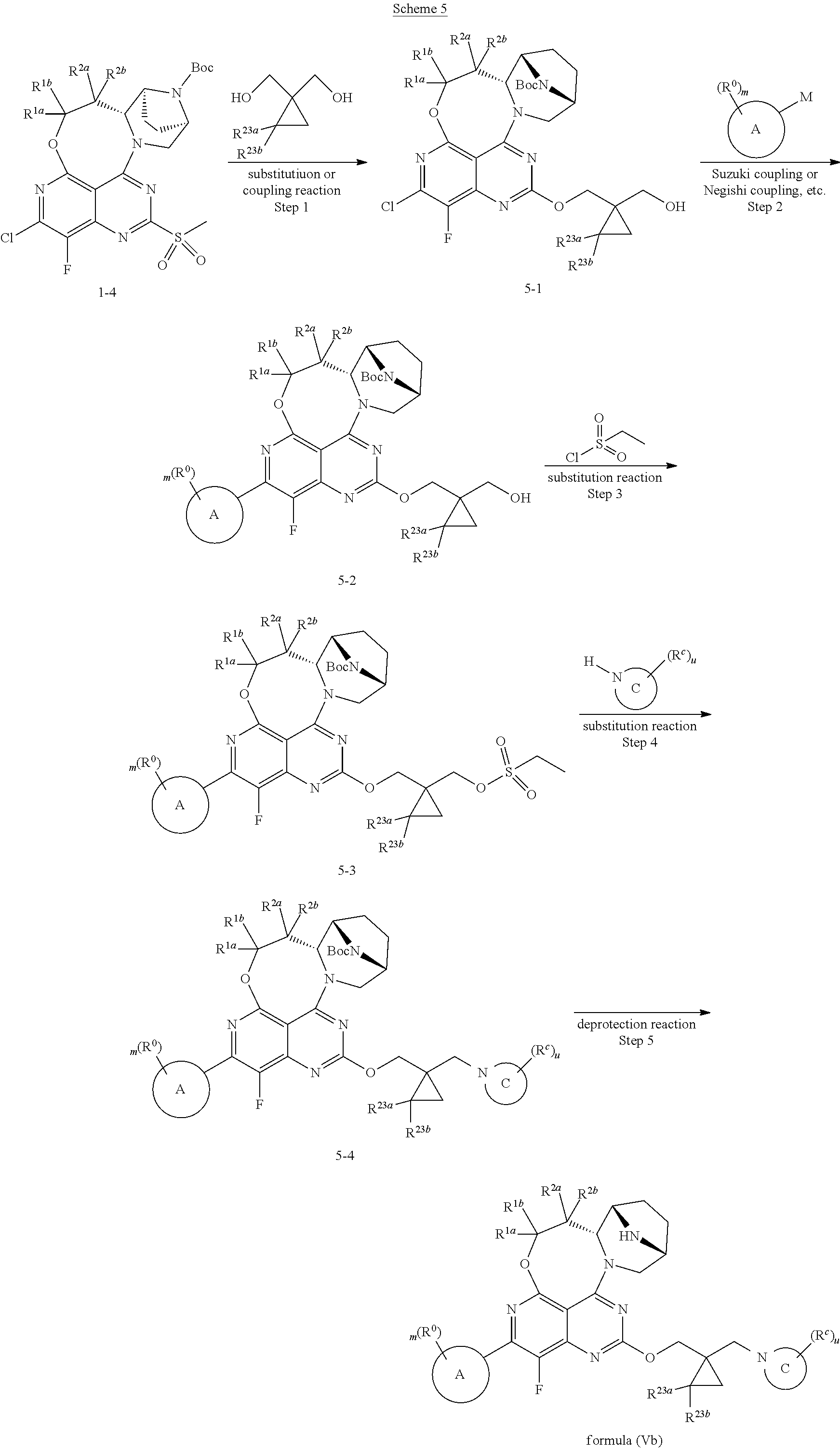

As shown in Scheme 5, in some embodiments, provided herein are methods for preparing the compounds defined as formula (Vb). Compound 1-4 is converted to compound 5-1 followed by substitution or coupling reactions (e.g., LIHMDS); compound 5-1 further undergoes metal catalyzed cross-coupling reaction such as Suzuki, Negishi, or Stille coupling (e.g. Pd(dtbpf)Cl₂, K₃PO₄, 1,4-dioxane, water for Suzuki coupling) to obtain compound 5-2, wherein M can be boronic acid, boronic ester, a metal (such as Zn), tributyltin, etc.; then compound 5-2 is converted to compound 5-3 followed by substitution reactions (e.g., DIEA); then compound 5-3 is converted to compound 5-4 followed by substitution or coupling reactions (e.g., ACN, TEA); finally, compound 5-4 containing protection group is then deprotected (e.g., HCU/dioxane to deprotect Boc group) to yield the compound defined as formula (Vb).

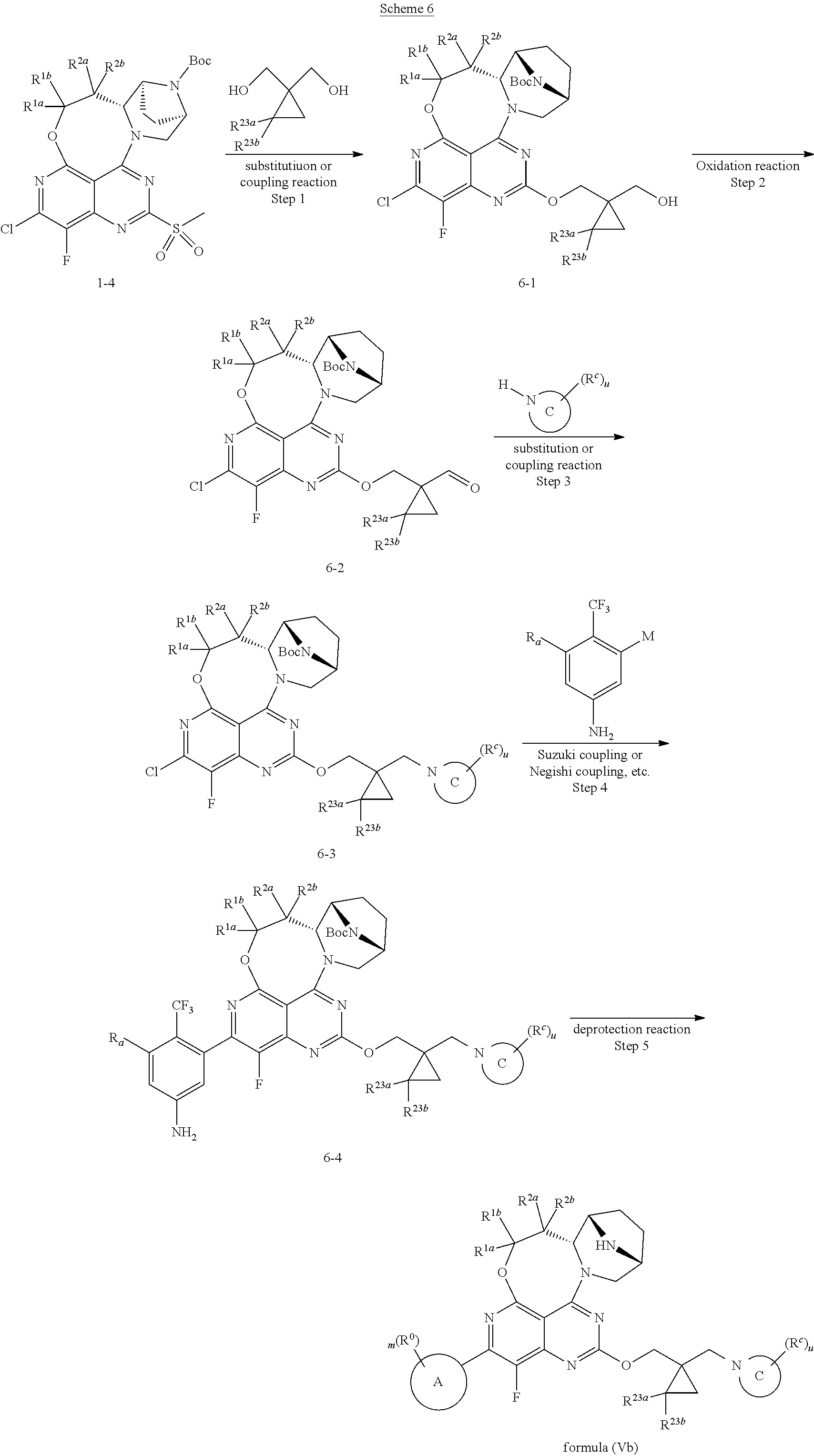

As shown in Scheme 6, in some embodiments, provided herein is another method for preparing the compounds defined as formula (Vb). Compound 1-4 is converted to compound 6-1 followed by substitution or coupling reactions (e.g., LHMDS); then compound 6-1 is converted to compound 6-2 followed by oxidation reactions (e.g., DMP); then compound 6-2 is converted to compound 6-3 followed by substitution or coupling reactions (e.g., STAB, DCE); compound 6-3 further undergoes metal catalyzed cross-coupling reaction such as Suzuki, Negishi, or Stille coupling (e.g. Pd(dtbpf)Cl₂, K₃PO₄, 1,4-dioxane, water for Suzuki coupling) to obtain compound 6-4, wherein M can be boronic acid, boronic ester, a metal (such as Zn), tributyltin, etc.; finally, compound 6-4 containing protection group is then deprotected (e.g., HCl/dioxane to deprotect Boc group) to yield the compound defined as formula (Vb).

The present embodiments can be understood more fully by reference to the detailed description and examples, which are intended to exemplify non-limiting embodiments.

### EXAMPLES

The examples below are intended to be purely exemplary and should not be considered to be limiting in any way. Unless otherwise specified, the experimental methods in the Examples described below are conventional methods. Unless otherwise specified, the reagents and materials are all commercially available. All solvents and chemicals employed are of analytical grade or chemical purity. Solvents are all redistilled before use. Anhydrous solvents are all prepared according to standard methods or reference methods. Silica gel (100-200 meshes) for column chromatography and silica gel (GF254) for thin-layer chromatography (TLC) are commercially available from Tsingdao Haiyang Chemical Co., Ltd. or Yantai Chemical Co., Ltd. of China; all were eluted with petroleum ether (60-90° C.)/ethyl acetate (v/v), and visualized by iodine or the solution of molybdphosphoric acid in ethanol unless otherwise specified. All extraction solvents, unless otherwise specified, were dried over anhydrous Na₂SO₄.

Unless otherwise indicated, the reactions set forth below were performed under a positive pressure of nitrogen or argon or with a drying tube in anhydrous solvents; the reaction flasks were fitted with rubber septa for the introduction of substrates and reagents via syringe; and glassware was oven dried and/or heat dried.

Unless otherwise indicated, column chromatography purification was conducted on a Biotage system (Manufacturer: Dyax Corporation) having a silica gel column or on a silica SepPak cartridge (Waters), or was conducted on a Teledyne Isco Combiflash purification system using prepacked silica gel cartridges.

¹H NMR spectra were recorded on a Varian instrument operating at 400 MHz or 500 MHz with TMS (tetramethylsilane) as the internal standard. ¹H-NMR spectra were obtained using CDCl₃, CD₂Cl₂, CD₃OD, D₂O, d₆-DMSO, d₆-acetone or (CD₃)₂CO as solvent and tetramethylsilane (0.00 ppm) or residual solvent (CDCl₃: 7.25 ppm; CD₃OD: 3.31 ppm; D₂O: 4.79 ppm; d₆-DMSO: 2.50 ppm; d₆-acetone: 2.05; (CD₃)₂CO: 2.05) as the reference standard. When peak multiplicities are reported, the following abbreviations are used: s (singlet), d (doublet), t (triplet), q (quartet), qn (quintuplet), sx (sextuplet), m (multiplet), br (broadened), dd (doublet of doublets), dt (doublet of triplets). Coupling constants, when given, are reported in Hertz (Hz).

LC/MS data was recorded by using Agilent1100,1200 High Performance Liquid Chromatography-Ion Trap Mass Spectrometer (LC-MSD Trap) equipped with a diode array detector (DAD) detected at 214 nm and 254 nm, and an ion trap (ESI source). All compound names except the reagents were generated by ChemDraw® 19.1.

In the following examples, the following abbreviations are used:
AcOH Acetic acid
Aq. Aqueous
9-BBN 9-Borabicyclo[3.3.1]nonane
BINAP 2,2′-bis(diphenylphosphino)-1,1′-binaphthalene
Brine Saturated aqueous sodium chloride solution
Bn Benzyl
BnBr Benzyl Bromide
Boc Tert-butoxycarbonyl
BOP Benzotriazol-1-yl-oxy-tris-(dimethylamino)phosphonium hexa-fluorophosphate
CH₂Cl₂ or DCM Dichloromethane
Cs₂CO₃ Cesium carbonate
DAST Diethylaminosulfur trifluoride
DCE 1,2-Dichloroethane
DCM Dichloromethane
DMF N,N-Dimethylformamide
Dppf 1,1′-bis(diphenylphosphino)ferrocene
DBU 1,8-diazabicyclo[5.4.0]undec-7-ene
DHP 3,4-Dihydro-2H-pyran
DIEA or DIPEA N,N-diisopropylethylamine
DMAP 4-N,N-dimethylaminopyridine
DMB (2,4-dimethoxyphenyl)methanamine
Dess-Martin/DMP Dess-Martin Periodinane
DMF N,N-dimethylformamide
DMF-DMA N,N-Dimethylformamide dimethyl acetal purum
DMSO Dimethyl sulfoxide
DMEDA Dimethyl Ethylene Diamine
EDCI 1-Ethyl-3-(3-dimethylaminopropyl)carbodiimide hydrochloride
EtOAc or EA Ethyl acetate
EtOH Ethanol
Et₃SiH Triethyl silhydride
Et₂O or ether Diethyl ether
g Grams
h or hr Hour
HATU O-(7-Azabenzotriazol-1-yl)-N,N,N′,N′-tetramethyluronium hexafluorophosphate
Hex Hexane
HCl Hydrochloric acid
HMDS Hexamethyldisilazane
HOBT 1-Hydroxybenzotriazole
HPLC High-performance liquid chromatography
IBX 2-Iodylbenzoic acid
i-PrOH Isopropyl alcohol
LCMS Liquid chromatography-mass spectrometry
LDA Lithium diisopropylamide
LiHMDS Lithium Bis(trimethylsilyl)amide
KHMDS Potassium bis(trimethylsilyl)amide
K₃PO₄ Tripotassium phosphate
mg Milligrams
mL Milliliters
mmol Millimole
MeCN Acetonitrile
MeOH Methanol
Min Minutes
ms or MS Mass spectrum
MsCl Methanesulfonyl chloride
m-CPBA 2-chloranylbenzenecarboperoxoic acid
MOM Methoxymethyl
MPLC Medium Pressure Liquid Chromatography
Na₂SO₄ Sodium sulfate
NaBH(OAc)₃/STAB Sodium triacetyl borohydride
NaH Sodium hydride
NaHMDS Sodium bis(trimethylsilyl)amide
NBS N-Bromosuccinimide
NCS N-Chlorosuccinimide
NMO 4-Methylmorpholine N-oxide
NMP N-Methyl Pyrrolidone
PD Pharmacodynamic(s)
PE petroleum ether
PK Pharmacokinetic(s)
PMB (4-methoxyphenyl)methanamine
POCl₃ phosphorous oxychloride
PyBOP Benzotriazol-1-yl-oxytripyrrolidinophosphonium hexafluorophosphate
Pd(dppf)Cl₂ [1,1′-Bis(diphenylphosphino)ferrocene]dichloropalladium(II)
Pd(dtbpf)Cl₂ [1,1′-Bis(di-tert-butylphosphino)ferrocene]dichloropalladium(II)
Pd₂(dba)₃ Tris(dibenzylideneacetone)dipalladium
Prep Preparative
PTSA 4-Methylbenzenesulfonic acid
Rt or rt Room temperature
sat. Saturated
SEMCl (2-(Chloromethoxy)ethyl)trimethylsilane
TBSCl tert-Butyldimethylsilyl chloride
TBDPSCl tert-Butyl(chloro)diphenylsilane
TEA/Et3N triethylamine
t-BuOK Potassium tert-butoxide
t-BuONa Sodium tert-butoxide
T₃P n-Propylphosphonic cyclic anhydride
TIPS Triisopropylsilyl
TFA Trifluoroacetic acid
TFAA Trifluoroacetic anhydride
THF Tetrahydrofuran
TLC thin layer chromatography
μL Microliters

### Compound Synthesis

### Preparation of Intermediate 1: tert-butyl (1S,2R,5R)-2-(2-hydroxyethyl)-3,8-diazabicyclo[3.2.1]octane-8-carboxylate

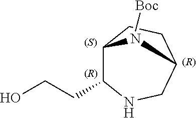

### Synthetic Route:

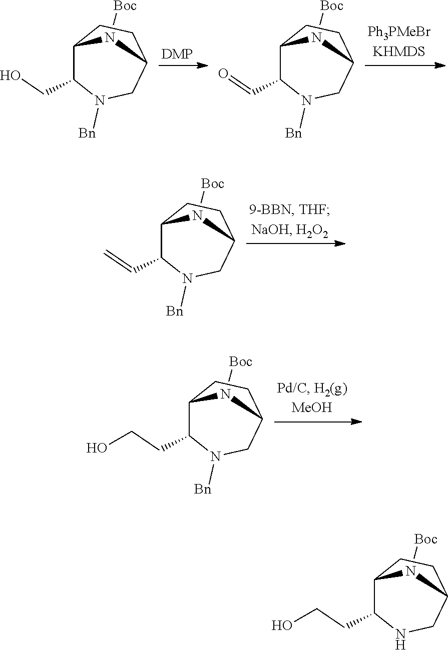 [SMILES: CC(C)(C)OC(=O)N1[C@@H]2CC[C@H]1[C@@H](CO)N(Cc1ccccc1)C2 ; CC(C)(C)OC(=O)N1[C@@H]2CC[C@H]1[C@@H](C=O)N(Cc1ccccc1)C2 ; C=C[C@@H]1[C@@H]2CC[C@H](CN1Cc1ccccc1)N2C(=O)OC(C)(C)C ; CC(C)(C)OC(=O)N1[C@@H]2CC[C@H]1[C@@H](CCO)N(Cc1ccccc1)C2 ; CC(C)(C)OC(=O)N1[C@@H]2CC[C@H]1[C@@H](CCO)NC2]

### Step 1: tert-butyl (1S,2S,5R)-3-benzyl-2-formyl-3,8-diazabicyclo[3.2.1]octane-8-carboxylate

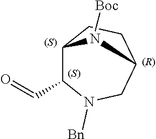

To a 250-mL round-bottom flask was added tert-butyl (1S,2S,5R)-3-benzyl-2-(hydroxymethyl)-3,8-diazabicyclo[3.2.1]octane-8-carboxylate (1.55 g, 4.65 mmol), DMP (2.37 g, 5.58 mmol), and DCM (50 mL). The reaction mixture was stirred at rt for 2 h, which was then concentrated in vacuo. The crude was purified by column chromatography (PE/EA=0-12%), affording the title product (1.02 g, 68% yield) as a colorless oil.

### Step 2: tert-butyl (1S,2R,5R)-3-benzyl-2-vinyl-3,8-diazabicyclo[3.2.1]octane-8-carboxylate

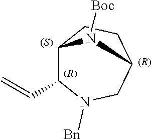

To a 50-mL round-bottom flask was added PPh₃MeBr (748 mg, 2.11 mmol), THF (6 mL), and KHMDS (1 M, 1.76 mL, 1.76 mmol). The reaction mixture was stirred at rt for 30 min, followed by addition of tert-butyl (1S,2S,5R)-3-benzyl-2-formyl-3,8-diazabicyclo[3.2.1]octane-8-carboxylate (580 mg, 1.76 mmol) in THF (6 mL) dropwise at rt. The resulting mixture was stirred at rt for 2 h. The reaction was then quenched by addition of sat. NH₄Cl (50 mL). The mixture was extracted by EtOAc (20 mL) for 3 times. The organic phase was combined, dried with Na₂SO₄, and filtered through Celite. The mixture was concentrated in vacuo. The resulting crude was purified by column chromatography (PE/EA=0-12%), affording the title compound (400 mg, 69% yield) as a white solid.

### Step 3: tert-butyl (1S,2R,5R)-3-benzyl-2-(2-hydroxyethyl)-3,8-diazabicyclo[3.2.1]octane-8-carboxylate

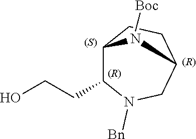

To a 100-mL round-bottom flask was added tert-butyl (1S,2R,5R)-3-benzyl-2-vinyl-3,8-diazabicyclo[3.2.1]octane-8-carboxylate (1 g, 0.0030 mol), 9-BBN (0.5 M, 36.5 mL, 0.018 mol), and THF (10 mL). The reaction mixture was stirred at 50° C. for 2 h under N₂ atmosphere. After the reaction is complete as indicated by LCMS, aq. NaOH (3 M, 7 ml) and H₂O₂ (7 mL) was added dropwise at 0° C. The resulting mixture was stirred at rt for 2 h. The mixture was diluted with H₂O (50 mL) and extracted by EtOAc (30 mL) for 3 times. The organic phase was combined, dried over Na₂SO₄, and concentrated in vacuo. The crude was purified by column chromatography (PE/EA=0-23%), affording the title compound (900 mg, 85% yield) as a colorless oil.

### Step 4: tert-butyl (1S,2R,5R)-2-(2-hydroxyethyl)-3,8-diazabicyclo[3.2.1]octane-8-carboxylate

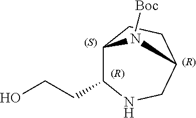

To a 100-mL round bottom flask was added tert-butyl (1S,2R,5R)-3-benzyl-2-(2-hydroxyethyl)-3,8-diazabicyclo[3.2.1]octane-8-carboxylate (1.7 g, 4.91 mmol), Pd/C (10 wt %, 1.7 g), and MeOH (15 mL). The reaction was stirred at rt for 3 h under H₂ atmosphere. The reaction mixture was then filtered through Celite, and concentrated in vacuo, affording the title compound (1.1 g, 91% yield) as a colorless oil. MS (ESI, m/e) [M+H]⁺=257.10. ¹H NMR (300 MHz, DMSO-d6) δ 4.45 (d, J=4.8 Hz, 1H), 4.06-3.80 (m, 1H), 3.73 (d, J=6.6 Hz, 1H), 3.42 (q, J=11.1, 8.8 Hz, 2H), 3.06-2.63 (m, 2H), 2.09 (t, J=33.2 Hz, 1H), 1.87-1.48 (m, 4H), 1.39 (s, 9H).

### Preparation of Intermediate 2: tert-butyl (6aR,7S,10R)-2-chloro-1-fluoro-13-(methylsulfonyl)-5,6,6a,7,8,9,10,11-octahydro-4-oxa-3,11a,12,14,15-pentaaza-7,10-methanocyclohepta[4,5]cycloocta[1,2,3-de]naphthalene-15-carboxylate

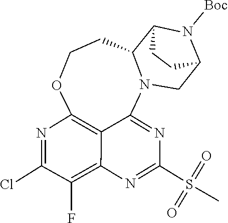 [SMILES: CC(C)(C)OC(=O)N1[C@@H]2CC[C@H]1[C@H]1CCOc3nc(Cl)c(F)c4nc(S(C)(=O)=O)nc(c34)N1C2]

### Synthetic Route

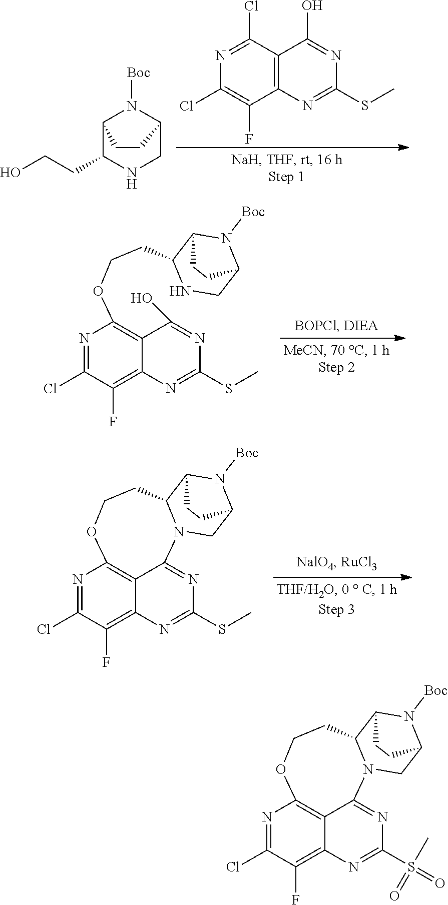

### Step 1: tert-butyl (1S,2R,5R)-2-(2-((7-chloro-8-fluoro-4-hydroxy-2-(methylthio)pyrido[4,3-d]pyrimidin-5-yl)oxy)ethyl)-3,8-diazabicyclo[3.2.1]octane-8-carboxylate

To a mixture of tert-butyl (1S,2R,5R)-2-(2-hydroxyethyl)-3,8-diazabicyclo[3.2.1]octane-8-carboxylate (1.53 g, 6 mmol) and 5,7-dichloro-8-fluoro-2-(methylthio)pyrido[4,3-d]pyrimidin-4-ol (1.68 g, 6 mmol) in THF (150 mL) was added NaH (1.2 g, 30 mmol). The mixture was stirred at room temperature for 20 hours. The reaction was then quenched by addition of saturated ammonium chloride aqueous solution (50 mL) and water (50 mL). The resulting mixture was extracted by EtOAc (50 mL) for 3 times. The organic phase was combined, dried over Na₂SO₄, and concentrated in vacuo to give the crude title product (4 g). MS (ESI, m/e) [M+H]⁺ 499.8.

### Step 2: tert-butyl (6aR,7S,10R)-2-chloro-1-fluoro-13-(methylthio)-5,6,6a,7,8,9,10,11-octahydro-4-oxa-3,11a,12,14,15-pentaaza-7,10-methanocyclohepta[4,5]cycloocta[1,2,3-de]naphthalene-15-carboxylate

To a solution of tert-butyl (1S,2R,5R)-2-(2-((7-chloro-8-fluoro-4-hydroxy-2-(methylthio)pyrido[4,3-d]pyrimidin-5-yl)oxy)ethyl)-3,8-diazabicyclo[3.2.1]octane-8-carboxylate (4 g, 8 mmol) in MeCN (220 mL) was added BOP—Cl (4.1 g, 5.1 mmol) and DIPEA (980 mg, 7.6 mmol). The resulting mixture was stirred at 70° C. for 1 hour. The mixture was cooled to room temperature and concentrated in vacuo. The crude was purified by column chromatography (DCM/EtOAc=5/1) to give the title product (1.6 g). MS (ESI, m/e) [M+H]⁺ 481.9.

### Step 3: tert-butyl (6aR,7S,10R)-2-chloro-1-fluoro-13-(methylsulfonyl)-5,6,6a,7,8,9,10,11-octahydro-4-oxa-3,11a,12,14,15-pentaaza-7,10-methanocyclohepta[4,5]cycloocta[1,2,3-de]naphthalene-15-carboxylate

A solution of tert-butyl (6aR,7S,10R)-2-chloro-1-fluoro-13-(methylthio)-5,6,6a,7,8,9,10,11-octahydro-4-oxa-3,11a,12,14,15-pentaaza-7,10-methanocyclohepta[4,5]cycloocta[1,2,3-de]naphthalene-15-carboxylate (378 mg, 0.8 mmol) in THF (28 mL) and water (7 mL) was added NaIO₄ (505 mg, 2.4 mmol) and RuCl₃ (16 mg, 0.08 mmol). The resulting mixture was stirred at 0° C. for 1 hour. The mixture was diluted by addition of water (20 mL), extracted by EtOAc (20 mL) for 3 times. The organic phase was combined and concentrated in vacuo to give the crude title compound (335 mg), which was used as is in next step. MS (ESI, m/e) [M+H]⁺ 514.2.

### Example 01: 3-((6aR,7S,10R)-13-((2,2-difluorotetrahydro-1H-pyrrolizin-7a(5H)-yl)methoxy)-1-fluoro-5,6,6a,7,8,9,10,11-octahydro-4-oxa-3,11a,12,14,15-pentaaza-7,10-methanocyclohepta[4,5]cycloocta[1,2,3-de]naphthalen-2-yl)-5-methyl-4-(trifluoromethyl)aniline

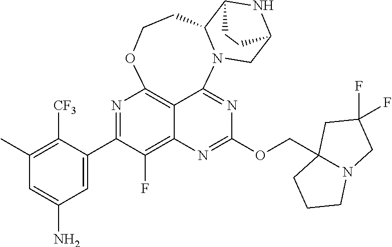 [SMILES: Cc1cc(N)cc(-c2nc3c4c(nc(OCC56CCCN5CC(F)(F)C6)nc4c2F)N2C[C@H]4CC[C@H](N4)[C@H]2CCO3)c1C(F)(F)F]

### Synthetic Route

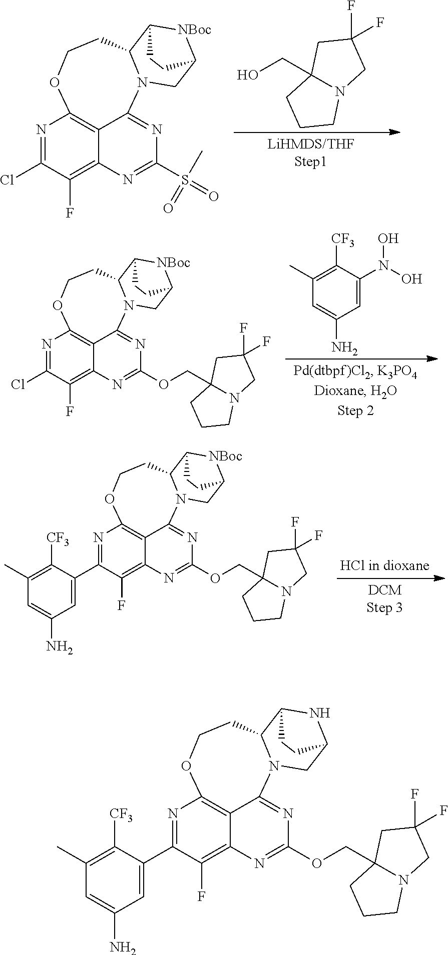

### Step 1: tert-butyl (6aR,7S,10R)-2-chloro-13-((2,2-difluorotetrahydro-1H-pyrrolizin-7a(5H)-yl)methoxy)-1-fluoro-5,6,6a,7,8,9,10,11-octahydro-4-oxa-3,11a,12,14,15-pentaaza-7,10-methanocyclohepta[4,5]cycloocta[1,2,3-de]naphthalene-15-carboxylate

To a solution of (1-((dimethylamino)methyl)-2,2-difluorocyclopropyl)methanol (17.7 mg, 0.1 mmol) in THF (5 mL) was added LiHMDS (1N, 0.1 mL, 0.1 mmol)) at room temperature. The resulting mixture was stirred for 1 h at room temperature. Then tert-butyl (6aR,7S,10R)-2-chloro-1-fluoro-13-(methylsulfonyl)-5,6,6a,7,8,9,10,11-octahydro-4-oxa-3,11a,12,14,15-pentaaza-7,10-methanocyclohepta[4,5]cycloocta[1,2,3-de]naphthalene-15-carboxylate (25.7 mg, 0.05 mmol) was added to the reaction. The resulting mixture was stirred for 1 hour at room temperature. After the reaction was complete as indicated by TLC, the reaction mixture was diluted with EtOAc (15 mL) and washed with brine (5 mL×3). The organic layer was dried over anhydrous Na₂SO₄ and filtered through Celite. The filtrate was concentrated to give the crude residue. The residue was purified by silica gel column chromatography, and eluted with 0 to 100% EtOAc in petroleum ether to give the title product (20 mg, 0.03 mmol). MS (ESI, m/e) [M+1]⁺ 611.2.

### Step 2: tert-butyl (6aR,7S,10R)-2-(5-amino-3-methyl-2-(trifluoromethyl)phenyl)-13-((2,2-difluorotetrahydro-1H-pyrrolizin-7a(5H)-yl)methoxy)-1-fluoro-5,6,6a,7,8,9,10,11-octahydro-4-oxa-3,11a,12,14,15-pentaaza-7,10-methanocyclohepta[4,5]cycloocta[1,2,3-de]naphthalene-15-carboxylate

To the mixture of tert-butyl (6aR,7S,10R)-2-chloro-13-((2,2-difluorotetrahydro-1H-pyrrolizin-7a(5H)-yl)methoxy)-1-fluoro-5,6,6a,7,8,9,10,11-octahydro-4-oxa-3,11a,12,14,15-pentaaza-7,10-methanocyclohepta[4,5]cycloocta[1,2,3-de]naphthalene-15-carboxylate (20 mg, 0.03 mmol), Pd(dtbpf)Cl₂ (6.51 mg, 0.01 mmol), and K₃PO₄ (21.2 mg, 0.1 mmol) in dioxane (5 mL) and water (1 mL) was added (5-amino-3-methyl-2-(trifluoromethyl)phenyl)boronic acid (21.9 mg, 0.01 mmol) at room temperature. The resulting mixture was stirred at 100° C. for 4 hours under N₂ atmosphere. After the reaction was complete as indicated by TLC, the reaction mixture was diluted with DCM (20 mL), washed with brine (10 mL×3). The organic layer was dried over anhydrous Na₂SO₄ and filtered through Celite. The filtrate was concentrated to give the crude residue. The residue was purified by silica gel column chromatography, eluted with 0 to 10% MeOH in DCM to give the title product (22.5 mg, 0.03 mmol). MS (ESI, m/e) [M+1]⁺ 750.3.

### Step 3: 3-((6aR,7S,10R)-13-((2,2-difluorotetrahydro-1H-pyrrolizin-7a(5H)-yl)methoxy)-1-fluoro-5,6,6a,7,8,9,10,11-octahydro-4-oxa-3,11a,12,14,15-pentaaza-7,10-methanocyclohepta[4,5]cycloocta[1,2,3-de]naphthalen-2-yl)-5-methyl-4-(trifluoromethyl)aniline

To a solution of tert-butyl (6aR,7S,10R)-2-(5-amino-3-methyl-2-(trifluoromethyl)phenyl)-13-((2,2-difluorotetrahydro-1H-pyrrolizin-7a(5H)-yl)methoxy)-1-fluoro-5,6,6a,7,8,9,10,11-octahydro-4-oxa-3,11a,12,14,15-pentaaza-7,10-methanocyclohepta[4,5]cycloocta[1,2,3-de]naphthalene-15-carboxylate (22.5 mg, 0.03 mmol) in DCM (3 mL) was added 4 N HCl in dioxane (1 mL) at room temperature. The resulting mixture was stirred at room temperature for 1 hour. After the reaction was complete as indicated by TLC, the reaction mixture was concentrated to give the crude residue. The residue was purified by prep-HPLC (Column: Waters SunFire C18, 19*150 mm, 5 μm; Mobile Phase A: H₂O (0.1% FA), Mobile Phase B: MeCN (0.1% FA); Flow Rate: 17 mL/min; Temperature: 25° C.; Detector: UV 254 & 280 nm; Gradient: 0 to 10% B in 1 min, 10 to 25% B within 1 to 11 min, 25 to 100% B within 11 to 13 min) to give the title product (10.0 mg). ¹H NMR (500 MHz, CD₃OD) δ 6.69 (s, 1H), 6.44 (s, 1H), 4.30-3.76 (m, 9H), 3.44-3.42 (m, 1H), 3.15-3.10 (m, 2H), 2.84-2.54 (m, 2H), 2.42 (s, 3H), 2.38-1.88 (m, 11H). MS (ESI, m/e) [M+1]⁺ 650.5.

The following Examples 02-06 were prepared in similar fashion as Example 01 by replacing (2,2-difluorotetrahydro-1H-pyrrolizin-7a(5H)-yl)methanol with the corresponding alkyl alcohols.

| | | | | MS |
|---|---|---|---|---|
| | | | | (ESI, |
| Ex- | | | | m/e) |
| am- | | | ¹H NMR (500 MHz, | [M + |
| ple | Structure | Compound Name | CD₃OD) δ | H]⁺ |
| 02 | 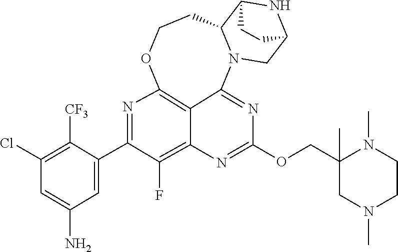 [SMILES: CN1CCN(C)C(C)(COc2nc3c4c(nc(-c5cc(N)cc(Cl)c5C(F)(F)F)c(F)c4n2)OCC[C@@H]2[C@@H]4CC[C@H](CN32)N4)C1] (isomer 1) | 3-chloro-5-((6aR,7S,10R)-1- fluoro-13-((1,2,4- trimethylpiperazin-2-yl)methoxy)- 5,6,6a,7,8,9,10,11-octahydro-4- oxa-3,11a,12,14,15-pentaaza-7,10- methanocyclohepta[4,5]cycloocta [1,2,3-de]naphthalen-2-yl)-4- (trifluoromethyl)aniline | 6.89 (s, 1H), 6.55-6.49 (m, 1H), 4.58-4.57 (m, 3H), 4.38-3.74 (m, 7H), 3.01-2.62 (m, 5H), 2.46 (s, 3H), 2.34 (s, 3H), 2.17-1.91 (m, 6H), 1.29 (s, 3H). | 651.5 |
| 03 | 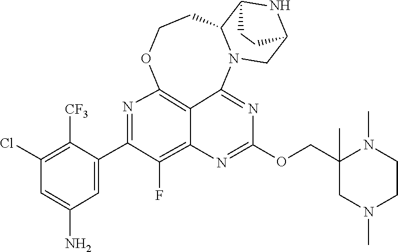 [SMILES: CN1CCN(C)C(C)(COc2nc3c4c(nc(-c5cc(N)cc(Cl)c5C(F)(F)F)c(F)c4n2)OCC[C@@H]2[C@@H]4CC[C@H](CN32)N4)C1] (isomer 2) | 3-chloro-5-((6aR,7S,10R)-1- fluoro-13-((1,2,4- trimethylpiperazin-2-yl)methoxy)- 5,6,6a,7,8,9,10,11-octahydro-4- oxa-3,11a,12,14,15-pentaaza-7,10- methanocyclohepta[4,5]cycloocta [1,2,3-de]naphthalen-2-yl)-4- (trifluoromethyl)aniline | 6.91 (s, 1H), 6.54-6.53 (m, 1H), 4.58-4.57 (m, 3H), 4.38-3.74 (m, 7H), 3.01-2.62 (m, 5H), 2.46 (s, 3H), 2.34 (s, 3H), 2.17-1.91 (m, 6H), 1.30 (s, 3H). | 651.5 |
| 04 | 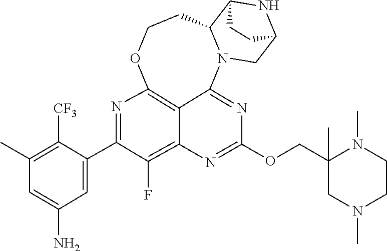 [SMILES: Cc1cc(N)cc(-c2nc3c4c(nc(OCC5(C)CN(C)CCN5C)nc4c2F)N2C[C@H]4CC[C@H](N4)[C@H]2CCO3)c1C(F)(F)F] (isomer 1) | 3-((6aR,7S,10R)-1-fluoro-13- ((1,2,4-trimethylpiperazin-2- yl)methoxy)-5,6,6a,7,8,9,10,11- octahydro-4-oxa-3,11a,12,14,15- pentaaza-7,10- methanocyclohepta[4,5]cycloocta [1,2,3-de]naphthalen-2-yl)-5- methyl-4-(trifluoromethyl)aniline | 6.70 (s, 1H), 6.45-6.44 (m, 1H), 4.58-3.78 (m, 10H), 2.90-2.64 (m, 5H), 2.56 (s, 3H), 2.42 (s, 3H), 2.36 (s, 3H), 2.21-1.91 (m, 6H), 1.30 (s, 3H). | 631.5 |
| 05 | 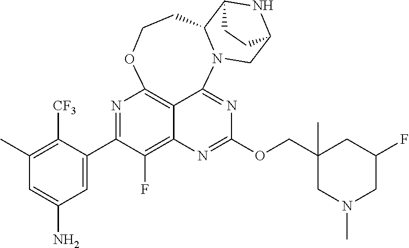 [SMILES: Cc1cc(N)cc(-c2nc3c4c(nc(OCC5(C)CC(F)CN(C)C5)nc4c2F)N2C[C@H]4CC[C@H](N4)[C@H]2CCO3)c1C(F)(F)F] | 3-((6aR,7S,10R)-1-fluoro-13-((5- fluoro-1,3-dimethylpiperidin-3- yl)methoxy)-5,6,6a,7,8,9,10,11- octahydro-4-oxa-3,11a,12,14,15- pentaaza-7,10- methanocyclohepta[4,5]cycloocta [1,2,3-de]naphthalen-2-yl)-5- methyl-4-(trifluoromethyl)aniline | 6.69(s, 1H), 6.46-6.45 (m, 1H), 4.85-3.88 (m, 11H), 2.78-2.65(m, 2H), 2.31-1.56 (m, 15H), 1.12 (s, 3H). | 634.5 |
| 06 | 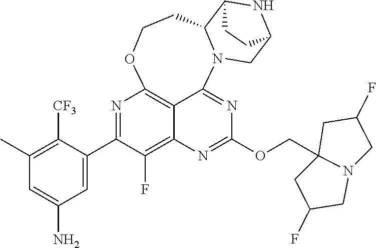 [SMILES: Cc1cc(N)cc(-c2nc3c4c(nc(OCC56CC(F)CN5CC(F)C6)nc4c2F)N2C[C@H]4CC[C@H](N4)[C@H]2CCO3)c1C(F)(F)F] | 3-((6aR,7S,10R)-13-((2,6- difluorotetrahydro-1H-pyrrolizin- 7a(5H)-yl)methoxy)-1-fluoro- 5,6,6a,7,8,9,10,11-octahydro-4- oxa-3,11a,12,14,15-pentaaza-7,10- methanocyclohepta[4,5]cycloocta [1,2,3-de]naphthalen-2-yl)-5- methyl-4-(trifluoromethyl)aniline | 6.69 (s, 1H), 6.43 (s, 1H), 5.40-5.26 (m, 2H), 4.41-3.80 (m, 10H), 3.31-3.16 (m, 3H), 2.46-1.88 (m, 13H). | 650.5 |

### Example 07: (3R,4R)-4-(((6aR,7S′,10R)-2-(5-amino-3-methyl-2-(trifluoromethyl)phenyl)-1-fluoro-5,6,6a,7,8,9,10,11-octahydro-4-oxa-3,11a,12,14,15-pentaaza-7,10-methanocyclohepta[4,5]cycloocta[1,2,3-de]naphthalen-13-yl)oxy)-N,N-dimethyltetrahydro-2H-pyran-3-amine

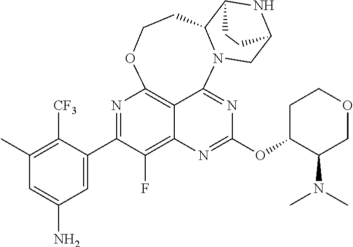

### Synthetic Route

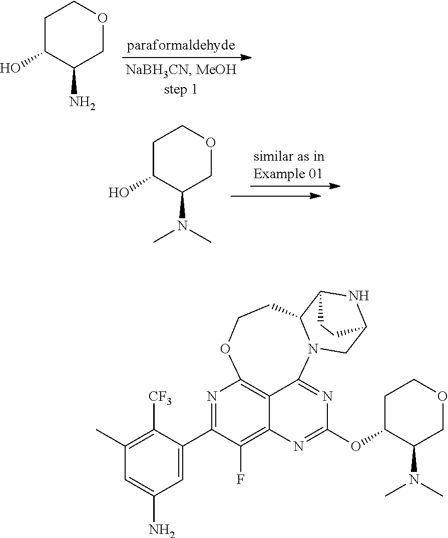

### Step 1: (3R,4R)-3-(dimethylamino)tetrahydro-2H-pyran-4-ol

To a mixture of (3R,4R)-3-aminotetrahydro-2H-pyran-4-ol (351.3 g, 3.0 mmol) in MeOH (30 mL) were added paraformaldehyde (909 mg, 30.0 mmol) and NaBH₃CN (366.0 mg, 6.0 mmol) at room temperature. The resulting mixture was stirred at room temperature for 16 hours. After the reaction was complete as indicated by LCMS, the reaction mixture was concentrated in vacuo to give the crude residue. The residue was purified by silica gel column chromatography, eluted with 0 to 10% MeOH in DCM to give the title product (200 mg, 1.38 mmol). MS (ESI, m/e) [M+1]⁺ 146.1.

Steps 2-4: Example 07 was prepared in similar fashion as Example 01 by replacing (2,2-difluorotetrahydro-1H-pyrrolizin-7a(5H)-yl)methanol with (3R,4R)-3-(dimethylamino)tetrahydro-2H-pyran-4-ol. ¹H NMR (500 MHz, CD₃OD) δ 6.69 (s, 1H), 6.46-6.44 (m, 1H), 5.44-5.40 (m, 1H), 4.30-3.72 (m, 10H), 3.55-3.51 (m, 1H), 3.17-3.16 (m, 1H), 2.49 (s, 6H), 2.42 (s, 3H), 2.18-1.80 (m, 8H). MS (ESI, m/e) [M+1]⁺ 618.5.

Examples 08 to 12: The following examples were prepared in a manner similar to that in Example 07 by replacing (3R,4R)-3-aminotetrahydro-2H-pyran-4-ol with the corresponding amino alcohols.

| | | | | MS |
|---|---|---|---|---|
| | | | | (ESI, |
| Ex- | | | | m/e) |
| am- | | | ¹H NMR (500 MHz, | [M + |
| ple | Structure | Compound Name | CD₃OD) δ | H]⁺ |
| 08 | 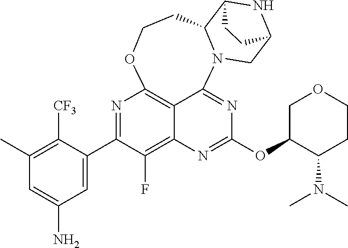 | (3R,4S)-3-(((6aR,7S,10R)-2-(5- amino-3-methyl-2- (trifluoromethyl)phenyl)-1-fluoro- 5,6,6a,7,8,9,10,11-octahydro-4- oxa-3,11a,12,14,15-pentaaza-7,10- methanocyclohepta[4,5]cycloocta [1,2,3-de]naphthalen-13-yl)oxy)- N,N-dimethyltetrahydro-2H- pyran-4-amine | 6.70 (s, 1H), 6.47- 6.46 (m, 1H), 5.43- 5.42 (m, 1H), 4.30- 3.35 (m, 12H), 2.42 (s, 6H), 2.36 (s, 3H), 2.25-1.84 (m, 8H). | 618.5 |
| 09 | 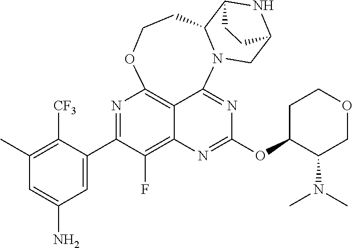 | (3S,4S)-4-(((6aR,7S,10R)-2-(5- amino-3-methyl-2- (trifluoromethyl)phenyl)-1-fluoro- 5,6,6a,7,8,9,10,11-octahydro-4- oxa-3,11a,12,14,15-pentaaza-7,10- methanocyclohepta[4,5]cycloocta [1,2,3-de]naphthalen-13-yl)oxy)- N,N-dimethyltetrahydro-2H- pyran-3-amine | 6.69 (s, 1H), 6.43 (s, 1H), 5.60-5.56 (m, 1H), 4.33-3.59 (m, 11H), 2.91-2.87 (m, 1H), 2.49 (s, 6H), 2.32 (s, 3H), 2.35-1.76 (m, 8H). | 618.5 |
| 10 | 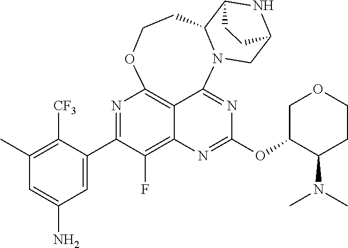 | (3S,4R)-3-(((6aR,7S,10R)-2-(5- amino-3-methyl-2- (trifluoromethyl)phenyl)-1-fluoro- 5,6,6a,7,8,9,10,11-octahydro-4- oxa-3,11a,12,14,15-pentaaza-7,10- methanocyclohepta[4,5]cycloocta [1,2,3-de]naphthalen-13-yl)oxy)- N,N-dimethyltetrahydro-2H- pyran-4-amine | 6.69 (s, 1H), 6.47- 6.46(m, 1H), 5.45- 5.40 (m, 1H), 4.58- 3.44 (m, 11H), 3.17- 3.16 (m, 1H), 2.49 (s, 6H), 2.42 (s, 3H), 2.18-1.80 (m, 8H). | 618.5 |
| 11 | 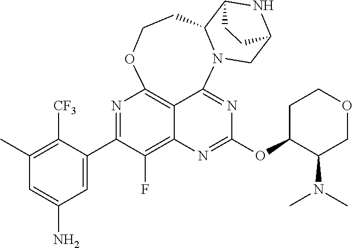 | (3R,4S)-4-(((6aR,7S,10R)-2-(5- amino-3-methyl-2- (trifluoromethyl)phenyl)-1-fluoro- 5,6,6a,7,8,9,10,11-octahydro-4- oxa-3,11a,12,14,15-pentaaza-7,10- methanocyclohepta[4,5]cycloocta [1,2,3-de]naphthalen-13-yl)oxy)- N,N-dimethyltetrahydro-2H- pyran-3-amine | 6.69 (s, 1H), 6.46- 6.44(m, 1H), 5.90- 5.86 (m, 1H), 4.31- 3.75 (m, 11H), 2.78- 2.77 (m, 1H), 2.47- 2.42 (m,9H),2.18- 1.91 (m, 8H). | 618.5 |
| 12 | 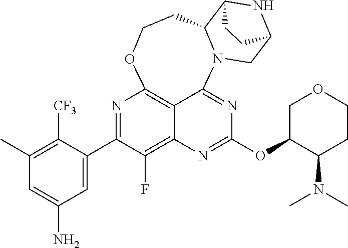 | (3R,4R)-3-(((6aR,7S,10R)-2-(5- amino-3-methyl-2- (trifluoromethyl)phenyl)-1-fluoro- 5,6,6a,7,8,9,10,11-octahydro-4- oxa-3,11a,12,14,15-pentaaza-7,10- methanocyclohepta[4,5]cycloocta [1,2,3-de]naphthalen-13-yl)oxy)- N,N-dimethyltetrahydro-2H- pyran-4-amine | 6.69 (s, 1H), 6.46- 6.41(m, 1H), 5.61- 5.60 (m, 1H), 4.30- 3.52 (m, 11H), 2.90- 2.87 (m, 1H), 2.53 (s, 6H), 2.42 (s, 3H), 2.22-1.84 (m, 8H). | 618.5 |
| 13 | 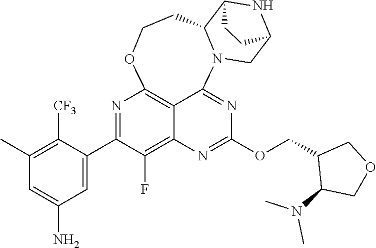 | (3S,4S)-4-((((6aR,7S,10R)-2-(5- amino-3-methyl-2- (trifluoromethyl)phenyl)-1-fluoro- 5,6,6a,7,8,9,10,11-octahydro-4- oxa-3,11a,12,14,15-pentaaza-7,10- methanocyclohepta[4,5]cycloocta [1,2,3-de]naphthalen-13- yl)oxy)methyl)-N,N- dimethyltetrahydrofuran-3-amine | (500 MHz, DMSO) δ 6.59 (s, 1H), 6.36- 6.35 (m, 1H), 5.87 (s, 2H), 4.46-3.79 (m, 9H), 3.63-3.45 (m, 4H), 2.72-2.64 (m, 2H), 2.36 (s, 3H), 2.17 (s, 6H), 1.99-1.56 (m, 6H). | 618.6 |
| 14 | 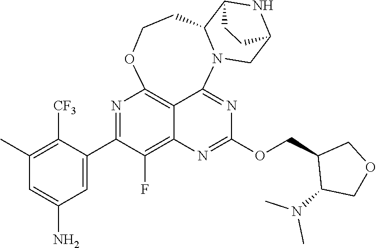 [SMILES: Cc1cc(N)cc(-c2nc3c4c(nc(OC[C@H]5COC[C@@H]5N(C)C)nc4c2F)N2C[C@H]4CC[C@H](N4)[C@H]2CCO3)c1C(F)(F)F] | (3R,4R)-4-((((6aR,7S,10R)-2-(5- amino-3-methyl-2- (trifluoromethyl)phenyl)-1-fluoro- 5,6,6a,7,8,9,10,11-octahydro-4- oxa-3,11a,12,14,15-pentaaza-7,10- methanocyclohepta[4,5]cycloocta [1,2,3-de]naphthalen-13- yl)oxy)methyl)-N,N- dimethyltetrahydrofuran-3-amine | (500 MHz, DMSO) δ 6.60 (s, 1H), 6.36- 6.35 (m, 1H), 5.88 (s, 2H), 4.46-3.79 (m, 9H), 3.63-3.45 (m, 4H), 2.72-2.64 (m, 2H), 2.36 (s, 3H), 2.18 (s, 6H), 1.99-1.54 (m, 6H). | 618.6 |
| 15 | 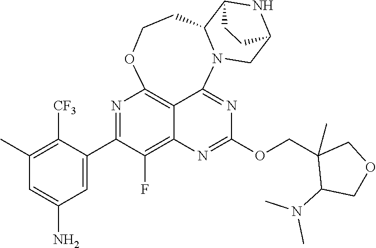 [SMILES: Cc1cc(N)cc(-c2nc3c4c(nc(OCC5(C)COCC5N(C)C)nc4c2F)N2C[C@H]4CC[C@H](N4)[C@H]2CCO3)c1C(F)(F)F] (mixture of two isomers) | 4-((((6aR,7S,10R)-2-(5-amino-3- methyl-2-(trifluoromethyl)phenyl)- 1-fluoro-5,6,6a,7,8,9,10,11- octahydro-4-oxa-3,11a,12,14,15- pentaaza-7,10- methanocyclohepta[4,5]cycloocta [1,2,3-de]naphthalen-13- yl)oxy)methyl)-N,N,4- trimethyltetrahydrofuran-3-amine | 6.70 (s, 1H), 6.45- 6.44 (m, 1H), 4.51- 3.63 (m, 13H), 2.98- 2.97 (m, 1H), 2.42 (s, 3H), 2.37 (s, 6H), 2.25-1.91 (m, 6H), 1.29 (s, 3H). | 632.6 |

### Example 16 and 17: 3-((6aR,7S,10R)-13-((1-((dimethylamino)methyl)-2,2-difluorocyclopropyl)methoxy)-1-fluoro-5,6,6a,7,8,9,10,11-octahydro-4-oxa-3,11a,12,14,15-pentaaza-7,10-methanocyclohepta[4,5]cycloocta[1,2,3-de]naphthalen-2-yl)-5-methyl-4-(trifluoromethyl)aniline

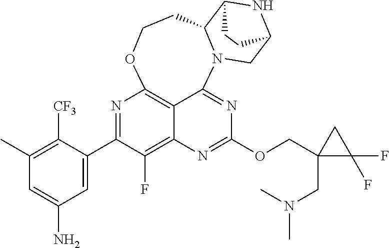 [SMILES: Cc1cc(N)cc(-c2nc3c4c(nc(OCC5(CN(C)C)CC5(F)F)nc4c2F)N2C[C@H]4CC[C@H](N4)[C@H]2CCO3)c1C(F)(F)F]

### Synthetic Route

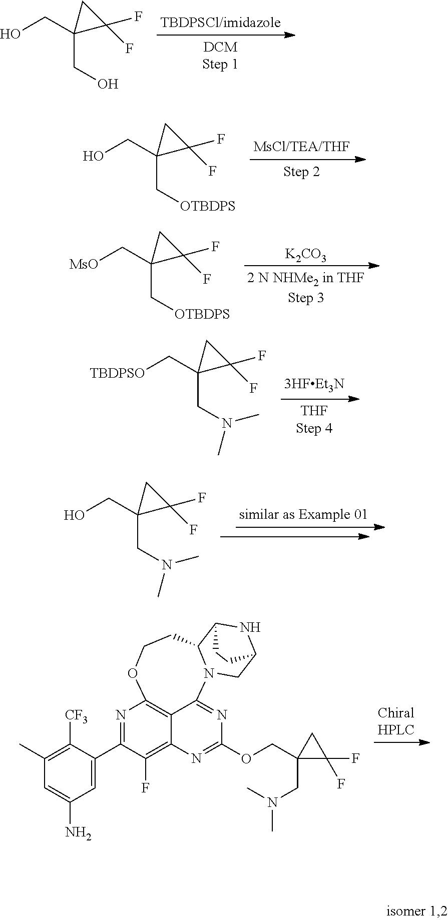

### Step 1: (1-(((tert-butyldiphenylsilyl)oxy)methyl)-2,2-difluorocyclopropyl)methanol

To a solution of (2,2-difluorocyclopropane-1,1-diyl)dimethanol (4.14 g, 30.0 mmol) in DCM (300 mL) were added TBDPSCl (8.22 g, 30.0 mmol) and imidazole (4.08 g, 60.0 mmol) at room temperature. The resulting mixture was stirred at room temperature for 16 hours. After completion of reaction as indicated by TLC, the reaction mixture was diluted with saturated NaHCO₃ (200 mL) and extracted with DCM (150 mL×3). The organic layer was dried over anhydrous Na₂SO₄ and filtered. The filtrate was concentrated in vacuo to give the crude residue. The residue was purified by silica gel column chromatography, eluted with 0 to 30% EtOAc in petroleum ether to give the title product (3.0 g, 7.97 mmol). MS (ESI, m/e) [M+23]⁺ 399.2.

### Step 2: (1-(((tert-butyldiphenylsilyl)oxy)methyl)-2,2-difluorocyclopropyl)methyl methanesulfonate

To the mixture of (1-(((tert-butyldiphenylsilyl)oxy)methyl)-2,2-difluorocyclopropyl)methanol (3.0 g, 7.97 mmol) in THF were added triethylamine (1.61 g, 15.9 mmol) and methanesulfonyl chloride (1.36 g, 12.0 mmol) at room temperature. The resulting mixture was stirred at 25° C. for 1 hour. After completion of reaction as indicated by TLC, the reaction mixture was concentrated in vacuo to give the crude product, which was directly used in the next step without further purification. MS (ESI, m/e) [M+23]⁺ 477.1.

### Step 3: 1-(1-(((tert-butyldiphenylsilyl)oxy)methyl)-2,2-difluorocyclopropyl)-N,N-dimehylmethanamine

To a mixture of (1-(((tert-butyldiphenylsilyl)oxy)methyl)-2,2-difluorocyclopropyl)methyl methanesulfonate (crude) in THF (50 mL) were added K₂CO₃ (2.76 g, 20 mmol) and dimethylamine (2N in THF, 100 mL) at room temperature. The resulting mixture was stirred at 65° C. for 16 hours in a sealed tube. The reaction mixture was then filtered through Celite. The filtrate was concentrated in vacuo to give the crude residue. The residue was purified by silica gel column chromatography, eluted with 0 to 70% EtOAc in petroleum ether to give the title product (2.4 g, 5.95 mmol). MS (ESI, m/e) [M+1]⁺ 404.2.

### Step 4: (1-((dimethylamino)methyl)-2,2-difluorocyclopropyl)methanol

To a solution of 1-(1-(((tert-butyldiphenylsilyl)oxy)methyl)-2,2-difluorocyclopropyl)-N,N-dimethylmethanamine (2.4 g, 5.95 mmol) in THF (50 mL) was added 3HF·Et₃N (1.61 g, 10 mmol) at room temperature. The resulting mixture was stirred at room temperature for 1 hour. After completion of reaction as indicated by TLC, the reaction mixture was concentrated in vacuo to give the crude residue. The residue was purified by silica gel column chromatography, eluted with 0 to 10% MeOH in DCM to give the title product (800 mg, 4.8 mmol). MS (ESI, m/e) [M+1]⁺ 166.1.

Steps 5-7: 3-((6aR,7S,10R)-13-((1-((dimethylamino)methyl)-2,2-difluorocyclopropyl) methoxy)-1-fluoro-5,6,6a,7,8,9,10,11-octahydro-4-oxa-3,11a,12,14,15-pentaaza-7,10-methanocyclohepta[4,5]cycloocta[1,2,3-de]naphthalen-2-yl)-5-methyl-4-(trifluoromethyl)aniline as a mixture of two isomers were prepared in similar fashion as Example 01 by replacing (2,2-difluorotetrahydro-1H-pyrrolizin-7a(5H)-yl)methanol with (1-((dimethylamino)methyl)-2,2-difluorocyclopropyl)methanol.

Step 8: The aforementioned two isomers (120 mg) was separated by chiral Prep-HPLC (Column: i-Cellulose-5, 21.2 mm×250 mm, 5 μm; Mobile Phase A: n-Hexane, Mobile Phase B: EtOH (with 0.2% 2M NH₃ in MeOH); Flow Rate: 18 mL/min; Temperature: 25° C.; Gradient: 50% B in 18 min; Retention time—isomer 1: 9.0 min; Retention time—isomer 2: 12.0 min) to give first eluting isomer (35 mg) as Example 16 and second eluting isomer (42 mg) as Example 17.

Example 16: ¹H NMR (500 MHz, CD₃OD) δ 6.68 (s, 1H), 6.46-6.41 (m, 1H), 4.62-4.54 (m, 2H), 4.36-4.00 (m, 5H), 3.69-3.44 (m, 2H), 2.82-2.81 (m, 1H), 2.45-2.42 (m, 4H), 2.28 (s, 6H), 2.09-1.80 (m, 6H), 1.68-1.65 (m, 1H), 1.42-1.37 (m, 1H). MS (ESI, m/e) [M+1]+638.5.

Example 17: ¹H NMR (500 MHz, CD₃OD) δ 6.68 (s, 1H), 6.45-6.44 (m, 1H), 4.58 (s, 2H), 4.36-4.00 (m, 5H), 3.38-3.44 (m, 2H), 2.86-2.83 (m, 1H), 2.45-2.42 (m, 4H), 2.27 (s, 6H), 2.16-1.80 (m, 6H), 1.68-1.65 (m, 1H), 1.42-1.37 (m, 1H). MS (ESI, m/e) [M+1]+638.5.

Examples 18: The following example was prepared in a manner similar to that in 3-((6aR,7S,10R)-13-((1-((dimethylamino)methyl)-2,2-difluorocyclopropyl)methoxy)-1-fluoro-5,6,6a,7,8,9,10,11-octahydro-4-oxa-3,11a,12,14,15-pentaaza-7,10-methanocyclohepta[4,5]cycloocta[1,2,3-de]naphthalen-2-yl)-5-methyl-4-(trifluoromethyl)aniline by replacing (5-amino-3-methyl-2-(trifluoromethyl) phenyl)boronic acid with the corresponding chloro-phenyl analogue.

| | | | | MS |
|---|---|---|---|---|
| | | | | (ESI, |
| Ex- | | | | m/e) |
| am- | | | 1H NMR (500 MHz, | [M + |
| ple | Structure | Compound Name | CD₃OD) δ | H]⁺ |
| 18 | 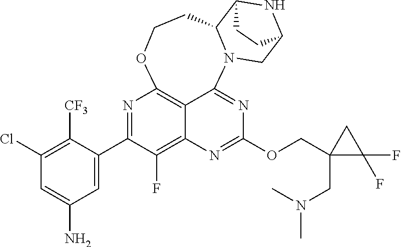 [SMILES: CN(C)CC1(COc2nc3c4c(nc(-c5cc(N)cc(Cl)c5C(F)(F)F)c(F)c4n2)OCC[C@@H]2[C@@H]4CC[C@H](CN32)N4)CC1(F)F] (mixture of two isomers) | 3-chloro-5-((6aR,7S,10R)-13-((1- ((dimethylamino)methyl)-2,2- difluorocyclopropyl)methoxy)-1- fluoro-5,6,6a,7,8,9,10,11- octahydro-4-oxa-3,11a,12,14,15- pentaaza-7,10- methanocyclohepta[4,5]cycloocta [1,2,3-de]naphthalen-2-yl)-4- (trifluoromethyl)aniline | 6.89 (s, 1H), 6.55- 6.49 (m, 1H), 4.60 (s, 2H), 4.37-3.79 (m, 7H), 2.94-2.52 (m, 2H), 2.37 (s, 6H), 2.12-1.29 (m, 8H). | 658.4 |

### Example 19: 3-((6aR,7S,10R)-13-((1-(azetidin-1-ylmethyl)-2,2-difluorocyclopropyl)methoxy)-1-fluoro-5,6,6a,7,8,9,10,11-octahydro-4-oxa-3,11a,12,14,15-pentaaza-7,10-methanocyclohepta[4,5]cycloocta[1,2,3-de]naphthalen-2-yl)-5-methyl-4-(trifluoromethyl)aniline

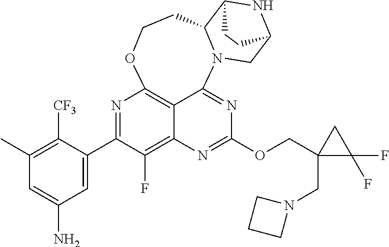 [SMILES: Cc1cc(N)cc(-c2nc3c4c(nc(OCC5(CN6CCC6)CC5(F)F)nc4c2F)N2C[C@H]4CC[C@H](N4)[C@H]2CCO3)c1C(F)(F)F]

### Synthetic Route

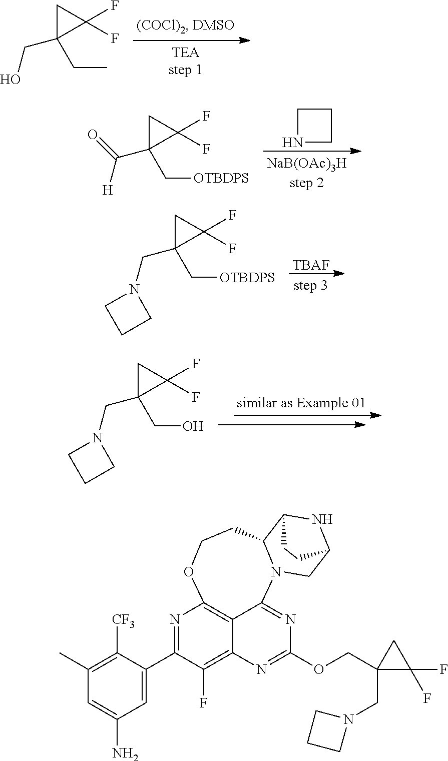

### Step 1: 1-(((tert-butyldiphenylsilyl)oxy)methyl)-2,2-difluorocyclopropane-1-carbaldehyde

To a solution of DMSO (1.86 g, 23.8 mmol) in DCM (20 mL) was added (COCl)₂ (7.3 mL, 14.28 mmol, 2M in DCM) at −78° C. The resulting mixture was stirred at −78° C. for 0.5 hour, then (1-(((tert-butyldiphenylsilyl)oxy)methyl)-2,2-difluorocyclopropyl)methanol (3.8 g, 9.52 mmol) was added. The result mixture was stirred for additional 0.5 hours at −78° C., followed by addition of TEA (6.6 mL, 47.6 mmol). After the reaction was complete as indicated by TLC, the reaction was quenched by addition of aq. NH₄Cl (10 mL). The mixture was extracted with DCM (50 mL×2). The organic layer was combined and washed with H₂O and brine, and then concentrated in vacuo. The crude was used in the next step without further purification. MS (ESI, m/e) no MS.

### Step 2: 1-((1-(((tert-butyldiphenylsilyl)oxy)methyl)-2,2-difluorocyclopropyl)methyl)azetidine

To a mixture of 1-(((tert-butyldiphenylsilyl)oxy)methyl)-2,2-difluorocyclopropane-1-carbaldehyde (500 mg, 1.82 mmol, crude) and azetidine (104 mg, 1.82 mmol) in DCM (20 mL) was added NaBH(OAc)₃ (462 mg, 31.82 mmol) at room temperature. The resulting mixture was stirred for 1 hour. The reaction was then quenched by addition of H₂O (50 mL). The mixture was extracted with DCM (30 mL×2). The organic layer was combined and washed with H₂O and brine, and then concentrated in vacuo. The crude was purified by column chromatography (eluting with DCM/MeOH=20/1) to afford the title compound as a colorless oil (210 mg, 79% yield over 2 steps). MS (ESI, m/e) [M+1]⁺ 416.5.

### Step 3: (1-(azetidin-1-ylmethyl)-2,2-difluorocyclopropyl)methanol

To a solution of 1-((1-(((tert-butyldiphenylsilyl)oxy)methyl)-2,2-difluorocyclopropyl)methyl)azetidine (200 mg, 0.48 mmol) in THF (5 mL) was added TBAF (0.58 mL, 0.58 mmol, 1 M in THF) at room temperature. The resulting mixture was stirred for 1 hour, and then concentrated in vacuo. The crude was purified by column chromatography (eluting with DCM/MeOH=15/1) to afford the title compound as a colorless oil (75 mg, 88% yield). MS (ESI, m/e) [M+1]⁺ 178.5.

Steps 4-6: Example 19 as a mixture of two isomers was prepared in similar fashion as Example 01 by replacing (2,2-difluorotetrahydro-1H-pyrrolizin-7a(5H)-yl)methanol with (1-(azetidin-1-ylmethyl)-2,2-difluorocyclopropyl)methanol. 1HNMR (500 MHz, CD₃OD) δ 6.70 (s, 1H), 6.45-6.43 (m, 1H), 4.69-4.61 (m, 1H), 4.56-4.46 (m, 1H), 4.42-4.25 (m, 2H, 4.20-4.06 (m, 2H), 3.96-3.76 (m, 6H, 3.30-3.18 (m, 3H), 2.44 (d, J=2.5 Hz, 3H), 2.36-2.28 (m, 2H, 2.24-1.80 (m, 6H, 1.76-1.61 (m, 2H). MS (ESI, m/e) [M+1]⁺ 650.5.

Examples 20-27: The following examples were prepared in a manner similar to that in Example 19 by replacing azetidine with the corresponding amines, followed by further separation of isomers via chiral Prep-HPLC (Column: i-Cellulose-5, 21.2 mm×250 mm, 5 μm; Mobile Phase A: n-Hexane, Mobile Phase B: EtOH (with 0.2% 2M NH₃ in MeOH); Flow Rate: 18 mL/min; Temperature: 25° C.; Gradient: 50% B in 18 min).

| | | | | MS |
|---|---|---|---|---|
| | | | | (ESI, |
| Ex- | | | | m/e) |
| am- | | Compound | 1H NMR (500 MHz, | [M + |
| ple | Structure | Name | CD₃OD) δ | H]⁺ |
| 20 | 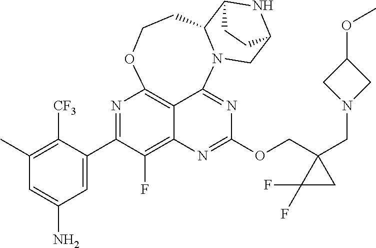 (isomer 1) | 3-((6aR,7S,10R)- 13-((2,2-difluoro- 1-((3-methoxyazetidin-1- yl)methyl)cyclopropyl) methoxy)- 1-fluoro-5,6,6a, 7,8,9,10,11- octahydro-4-oxa-3, 11a,12,14,15- pentaaza-7,10- methanocyclohepta [4,5]cycloocta [1,2,3-de]naphthalen- 2-yl)-5- methyl-4- (trifluoromethyl) aniline | 6.68 (s, 1H), 6.46- 6.41 (m, 1H), 4.57- 4.50 (m, 2H), 4.36- 4.27 (m, 3H), 4.09- 3.98 (m, 3H), 3.71- 3.61 (m, 4H), 3.22 (s, 3H), 3.08-3.03 (m,2H), 2.88-2.76 (m, 2H), 2.43-2.41 (m, 3H), 2.10-1.81 (m, 6H), 1.60-1.29 (m, 2H). | 680.5 |
| 21 | 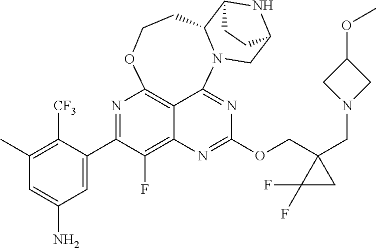 (isomer 2) | 3-((6aR,7S,10R)- 13-((2,2-difluoro- 1-((3-methoxyazetidin-1- yl)methyl)cyclopropyl) methoxy)- 1-fluoro-5,6,6a, 7,8,9,10,11- octahydro-4-oxa- 3,11a,12,14,15- pentaaza-7,10- methanocyclohepta [4,5]cycloocta[ 1,2,3-de] naphthalen-2-yl)-5- methyl-4- (trifluoromethyl) aniline | 6.69 (s, 1H), 6.47- 6.41 (m, 1H), 4.63- 4.60 (m, 1H), 4. 46- 4.09 (m, 5H), 3.63- 3.30 (m, 6H), 2.84- 2.77 (m,1H), 2.11-1.80 (m, 8H). 1.59-1.45 (m, 2H). | 680.5 |
| 22 | 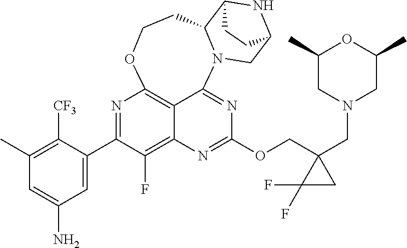 (isomer 1) | 3-((6aR,7S,10R)- 13-((1-(((2S,6R)- 2,6-dimethylmorpholino) methyl)- 2,2-difluorocyclopropyl) methoxy)- 1-fluoro-5,6,6a, 7,8,9,10,11- octahydro-4-oxa- 3,11a,12,14,15- pentaaza-7,10- methanocyclohepta [4,5]cycloocta [1,2,3-de] naphthalen-2-yl)-5- methyl-4- (trifluoromethyl)aniline | 6.69 (s, 1H), 6.46- 6.41 (m, 1H), 4.67- 4.61 (m, 2H), 4.37- 4.08 (m, 4H), 3.72- 3.46 (m, 4H), 2.84- 2.70 (m, 3H), 2.48- 2.38 (m, 4H), 2.10- 1.64 (m, 9H), 1.39- 1.34 (m, 1H), 1.11- 1.05 (m, 6H). | 708.5 |
| 23 | 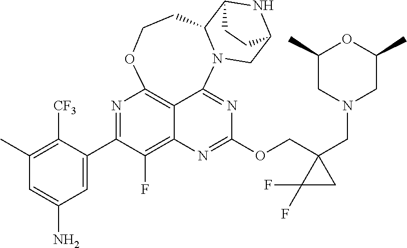 (isomer 2) | 3-((6aR,7S,10R)- 13-((1-(((2S,6R)- 2,6-dimethyl- morpholino)methyl)- 2,2-difluorocyclo- propyl)methoxy)- 1-fluoro-5,6,6a, 7,8,9,10,11- octahydro-4-oxa- 3,11a,12,14,15- pentaaza-7,10- methanocyclohepta [4,5]cycloocta [1,2,3-de] naphthalen-2-yl)-5- methyl-4- (trifluoromethyl)aniline | 6.69 (s, 1H), 6.46- 6.41 (m, 1H), 4.68- 4.61 (m, 2H), 4.41- 4.08 (m, 4H), 3.72- 3.44 (m, 4H), 2.85- 2.72 (m, 3H), 2.42- 2.38 (m, 4H), 2.10- 1.64 (m, 9H), 1.39- 1.34 (m, 1H), 1.10- 1.06 (m, 6H). | 708.5 |
| 24 | 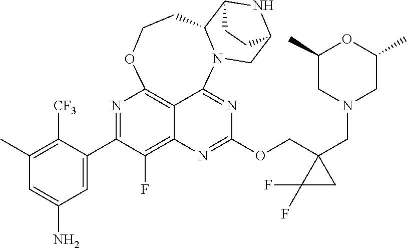 (isomer 1) | 3-((6aR,7S,10R)- 13-((1-(((2S,6S)- 2,6-dimethyl- morpholino)methyl)- 2,2-difluorocyclo- propyl)methoxy)- 1-fluoro-5,6,6a, 7,8,9,10,11- octahydro-4-oxa- 3,11a,12,14,15- pentaaza-7,10- methanocyclohepta [4,5]cycloocta [1,2,3-de] naphthalen-2-yl)-5- methyl-4-(trifluoro- methyl)aniline | 6.69 (s, 1H), 6.46- 6.41 (m, 1H), 4.77- 4.75 (m, 1H), 4.55- 4.53 (m, 1H), 4.38- 4.26 (m, 3H), 4.15- 4.05 (m, 2H), 3.98- 3.92 (m, 2H), 3.68- 3.59 (m, 2H), 2.81- 2.79 (m, 1H), 2.48- 2.34 (m, 6H), 2.22- 1.69 (m, 9H), 1.39- 1.35 (m, 1H), 1.16- 1.15 (m, 6H) | 708.5 |
| 25 | 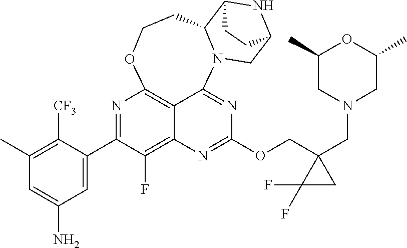 (isomer 2) | 3-((6aR,7S,10R)- 13-((1-(((2R,6R)- 2,6-dimethyl- morpholino)methyl)- 2,2-difluorocyclo- propyl)methoxy)- 1-fluoro-5,6,6a, 7,8,9,10,11- octahydro-4-oxa- 3,11a,12,14,15- pentaaza-7,10- methanocyclohepta [4,5]cycloocta [1,2,3-de] naphthalen-2-yl)-5- methyl-4-(trifluoro- methyl)aniline | 6.69 (s, 1H), 6.46- 6.42 (m, 1H), 4.66- 4.63 (m, 2H), 4.37- 4.09 (m, 4H), 3.93- 3.89 (m, 2H), 3.68- 3.55 (m, 2H), 2.76- 2.73 (m, 1H), 2.53- 2.50 (m, 2H), 2.46- 2.40 (m, 4H), 2.17- 1.64 (m, 9H), 1.39- 1.34 (m, 1H), 1.14- 1.12 (m, 6H). | 708.5 |
| 26 | 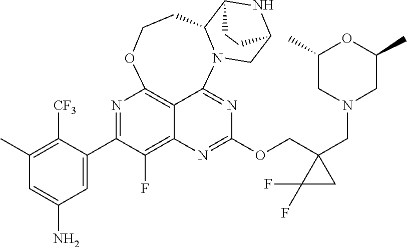 (isomer 1) | 3-((6aR,7S,10R)- 13-((1-(((2S,6S)- 2,6-dimethyl- morpholino)methyl)- 2,2-difluorocyclo- propyl)methoxy)- 1-fluoro-5,6,6a, 7,8,9,10,11- octahydro-4-oxa- 3,11a,12,14,15- pentaaza-7,10- methanocyclohepta [4,5]cycloocta [1,2,3-de] naphthalen-2-yl)-5- methyl-4-(trifluoro- methyl)aniline | 6.69 (s, 1H), 6.46- 6.42 (m, 1H), 4.70- 4.64 (m, 2H), 4.36- 4.26 (m, 2H), 4.15- 4.03 (m, 2H), 3.96- 3.90 (m, 2H), 3.72- 3.66 (m, 1H), 3.62- 3.51 (m, 1H), 2.73- 2.71 (m, 1H), 2.54- 2.52 (m, 2H), 2.45- 2.42 (m, 4H), 2.14- 1.76 (m, 8H), 1.68- 1.63 (m, 1H), 1.40- 1.33 (m, 1H), 1.14- 1.12 (m, 6H). | 708.5 |
| 27 | 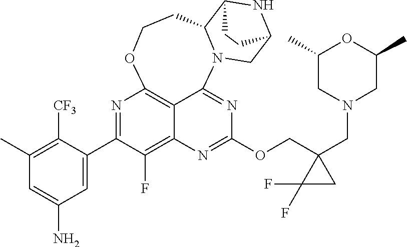 (isomer 2) | 3-((6aR,7S,10R)- 13-((1-(((2S,6S)- 2,6-dimethyl- morpholino)methyl)- 2,2-difluorocyclo- propyl)methoxy)- 1-fluoro-5,6,6a, 7,8,9,10,11- octahydro-4-oxa- 3,11a,12,14,15- pentaaza-7,10- methanocyclohepta [4,5]cycloocta [1,2,3-de] naphthalen-2-yl)-5- methyl-4-(trifluoro- methyl)aniline | 6.69 (s, 1H), 6.46- 6.42 (m, 1H), 4.75- 4.73 (m, 1H), 4.57- 4.55 (m, 1H), 4.37- 4.08 (m, 4H), 3.96- 3.94 (m, 2H), 3.68- 3.44 (m, 2H), 2.85- 2.82 (m, 1H), 2.48- 2.42 (m, 5H), 2.34- 2.32 (m, 1H), 2.22- 1.66 (m, 9H), 1.43- 1.34 (m, 1H), 1.16- 1.15 (m, 6H). | 708.5 |
| 28 | 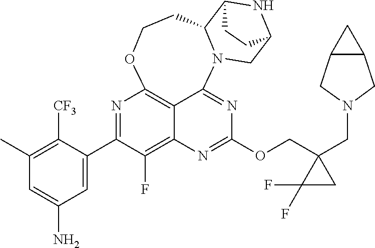 [SMILES: Cc1cc(N)cc(-c2nc3c4c(nc(OCC5(CN6CC7CC7C6)CC5(F)F)nc4c2F)N2C[C@H]4CC[C@H](N4)[C@H]2CCO3)c1C(F)(F)F] (isomer 1) | 3-((6aR,7S,10R)- 13-((1-((3- azabicyclo[3.1.0] hexan-3- yl)methyl)-2,2- difluorocyclopropyl) methoxy)-1- fluoro-5,6,6a, 7,8,9,10,11- octahydro-4-oxa- 3,11a,12,14,15- pentaaza-7,10- methanocyclohepta [4,5]cycloocta [1,2,3-de] naphthalen-2-yl)-5- methyl-4-(trifluoro- methyl)aniline | 6.69 (s, 1H), 6.45- 6.43 (m, 1H), 4.60- 4.54 (m, 2H), 4.36- 3.87 (m, 5H), 3.72- 3.57 (m, 2H), 2.97- 2.95 (m, 2H), 2.83- 2.81 (m, 1H), 2.63- 2.61 (m, 2H), 2.42- 2.35 (m, 5H), 2.10- 1.87 (m, 6H), 1.61- 1.54 (m, 1H), 1.36- 1.31 (m, 3H), 0.58- 0.56 (m, 1H), 0.30- 0.26 (m, 1H). | 676.5 |
| 29 | 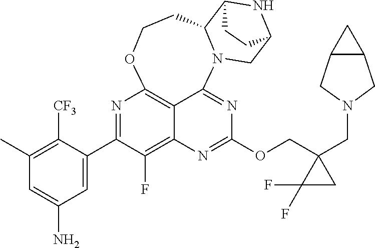 [SMILES: Cc1cc(N)cc(-c2nc3c4c(nc(OCC5(CN6CC7CC7C6)CC5(F)F)nc4c2F)N2C[C@H]4CC[C@H](N4)[C@H]2CCO3)c1C(F)(F)F] (isomer 2) | 3-((6aR,7S,10R)- 13-((1-((3- azabicyclo[3.1.0] hexan-3- yl)methyl)-2,2- difluorocyclopropyl) methoxy)-1- fluoro-5,6,6a, 7,8,9,10,11- octahydro-4-oxa- 3,11a,12,14,15- pentaaza-7,10- methanocyclohepta [4,5]cycloocta [1,2,3-de] naphthalen-2-yl)-5- methyl-4-(trifluoro- methyl)aniline | 6.69 (s, 1H), 6.45- 6.43 (m, 1H), 4.60- 4.55 (m, 2H), 4.36- 4.09 (m, 5H), 3.70- 3.57 (m, 2H), 2.97- 2.94 (m, 2H), 2.87- 2.85 (m, 1H), 2.59- 2.56 (m, 2H), 2.42- 2.34 (m, 5H), 2.10- 1.80 (m, 6H), 1.62- 1.54 (m, 1H), 1.34- 1.32 (m, 3H), 0.58- 0.56 (m, 1H), 0.29- 0.25 (m, 1H). | 676.5 |

### Example 30: 1-((6aR,7S,10R)-2-(5-amino-3-methyl-2-(trifluoromethyl)phenyl)-1-fluoro-S,6,6a,7,8,9,10,11-octahydro-4-oxa-3,11a,12,14,15-pentaaza-7,10-methanocyclohepta[4,5]cycloocta[1,2,3-de]naphthalen-13-yl)-N,N,3-trimethylazetidin-3-amine

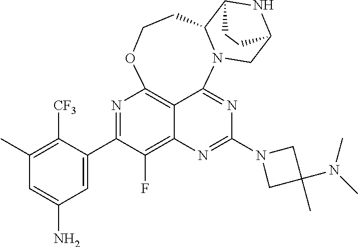

### Synthetic Route

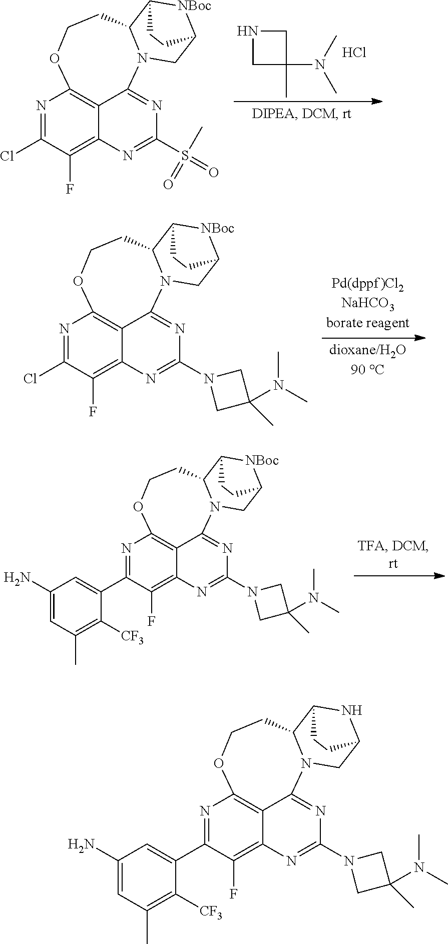

### Step 1: tert-butyl (6aR,7S,10R)-2-chloro-13-(3-(dimethylamino)-3-methylazetidin-1-yl)-1-fluoro-5,6,6a,7,8,9,10,11-octahydro-4-oxa-3,11a,12,14,15-pentaaza-7,10-methanocyclohepta [4,5]cycloocta[1,2,3-de]naphthalene-15-carboxylate

To a solution of tert-butyl (6aR,7S,10R)-2-chloro-1-fluoro-13-(methylsulfonyl)-5,6,6a,7,8,9,10,11-octahydro-4-oxa-3,11a,12,14,15-pentaaza-7,10-methanocyclohepta[4,5]cycloocta[1,2,3-de]naphthalene-15-carboxylate (120 mg, 0.233 mmol) in DCM (10 mL) was added N,N,3-trimethylazetidin-3-amine hydrogen chloride (140 mg, 0.934 mmol) and DIPEA (163 μL, 0.934 mmol) at room temperature. The resulting mixture was stirred at room temperature for 6 hours. After the reaction was complete as indicated by LCMS, silica gel was added to the reaction mixture. The resulting mixture was concentrated in vacuo. The residue was purified by flash chromatography (dry load, eluted with 0 to 100% EtOAc in petroleum ether) to give the title product (117 mg).

### Step 2: tert-butyl (6aR,7S,10R)-2-(5-amino-3-methyl-2-(trifluoromethyl)phenyl)-13-(3-(dimethylamino)-3-methylazetidin-1-yl)-1-fluoro-5,6,6a,7,8,9,10,11-octahydro-4-oxa-3,11a,12,14,15-pentaaza-7,10-methanocyclohepta[4,5]cycloocta[1,2,3-de]naphthalene-15-carboxylate

To a solution of tert-butyl (6aR,7S,10R)-2-chloro-13-(3-(dimethylamino)-3-methylazetidin-1-yl)-1-fluoro-5,6,6a,7,8,9,10,11-octahydro-4-oxa-3,11a,12,14,15-pentaaza-7,10-methanocyclohepta [4,5]cycloocta[1,2,3-de]naphthalene-15-carboxylate (62 mg, 0.113 mmol) in dioxane/H₂O (15 mL/3 mL) was added (5-amino-3-methyl-2-(trifluoromethyl) phenyl)boronic acid (76 mg, 0.35 mmol), Pd(dppf)Cl₂ (43 mg, 0.059 mmol) and NaHCO₃ (61 mg, 0.73 mmol) at room temperature. The mixture was degassed by bubbling nitrogen through for 5 minutes and was then stirred at 90° C. for 4 hours. After the reaction was complete as indicated by LCMS, silica gel was added to the reaction mixture. The resulting mixture was concentrated in vacuo. The residue was purified by silica gel column chromatography (dry load, eluted with 0 to 10% MeOH in DCM) to give the titled product (100 mg). MS (ESI, m/e) [M+H]⁺ 687.5.

### Step 3: 1-((6aR,7S,10R)-2-(5-amino-3-methyl-2-(trifluoromethyl)phenyl)-1-fluoro-5,6,6a,7,8,9,10,11-octahydro-4-oxa-3,11a,12,14,15-pentaaza-7,10-methanocyclohepta [4,5]cycloocta[1,2,3-de]naphthalen-13-yl)-N,N,3-trimethylazetidin-3-amine

To a solution of tert-butyl (6aR,7S,10R)-2-(5-amino-3-methyl-2-(trifluoromethyl)phenyl)-13-(3-(dimethylamino)-3-methylazetidin-1-yl)-1-fluoro-5,6,6a,7,8,9,10,11-octahydro-4-oxa-3,11a,12,14,15-pentaaza-7,10-methanocyclohepta[4,5]cycloocta[1,2,3-de]naphthalene-15-carboxylate (100 mg, 0.15 mmol) in DCM (20 mL) was added trifluoroacetic acid (4 mL) at room temperature. The mixture was stirred at room temperature for 4 hours. After the reaction was complete as indicated by LCMS, the mixture was concentrated in vacuo. The residue was redissolved in MeCN/DCM, followed by addition of 7 N ammonia methanol solution dropwise to adjust the pH to greater than 7. The mixture was concentrated in vacuo again and the residue was purified by pre-HPLC to give the titled product (28 mg). ¹H NMR (500 MHz, CD₃OD) δ 6.67 (s, 1H), 6.44-6.42 (m, 1H), 4.59 (s, 1H), 4.24 (s, 2H), 4.07-4.06 (m, 2H), 3.90-3.88 (m, 3H), 3.75-3.45 (m, 3H), 2.42-2.41 (m, 3H), 2.24 (s, 7H), 2.18-1.80 (m, 5H), 1.38 (s, 3H). MS (ESI, m/e) [M+H]⁺ 587.55.

Examples 31: The following examples were prepared in a manner similar to that in Example 30.

| | | | | MS |
|---|---|---|---|---|
| | | | | (ESI, |
| Ex- | | | | m/e) |
| am- | | | 1H NMR (500 MHz, | [M + |
| ple | Structure | Compound Name | CD₃OD) δ | H]⁺ |
| 31 | 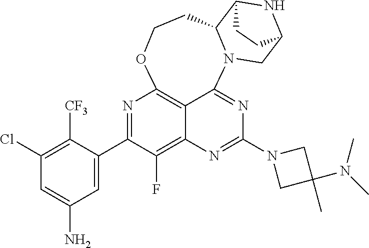 [SMILES: CN(C)C1(C)CN(c2nc3c4c(nc(-c5cc(N)cc(Cl)c5C(F)(F)F)c(F)c4n2)OCC[C@@H]2[C@@H]4CC[C@H](CN32)N4)C1] | 1-((6aR,7S,10R)-2-(5-amino-3- chloro-2-(trifluoromethyl)phenyl)- 1-fluoro-5,6,6a,7,8,9,10,11- octahydro-4-oxa-3,11a,12,14,15- pentaaza-7,10- methanocyclohepta[4,5]cycloocta [1,2,3-de]naphthalen-13-yl)-N,N,3- trimethylazetidin-3-amine | 6.87 (s, 1H), 6.52- 6.49 (m, 1H), 4.58- 3.67 (m, 11H), 2.25 (s, 6H), 2.15- 1.82 (m, 6H), 1.39 (s, 3H). | 607.4 |
| 32 | 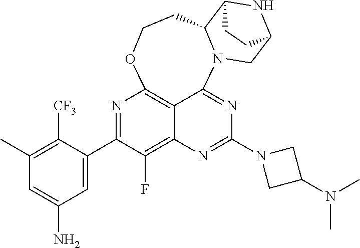 | 1-((6aR,7S,10R)-2-(5-amino-3- methyl-2-(trifluoromethyl)phenyl)- 1-fluoro-5,6,6a,7,8,9,10,11- octahydro-4-oxa-3,11a,12,14,15- pentaaza-7,10- methanocyclohepta[4,5]cycloocta [1,2,3-de]naphthalen-13-yl)-N,N- dimethylazetidin-3-amine | 6.68 (s, 1H), 6.44- 6.43(m, 1H), 4.38- 3.57 (m, 12H), 2.42 (s, 3H), 2.27 (s, 6H), 2.13-1.95 (m, 6H). | 573.5 |

Example 33-36: The following examples were prepared in a manner similar to that in Example 01 and Example 06 by replacing the corresponding alkyl alcohols as mixture of isomers with the alkyl alcohols as separated single enantiomer.

| | | | | MS |
|---|---|---|---|---|
| | | | | (ESI, |
| Ex- | | | | m/e) |
| am- | | | ¹H NMR (500 MHz, | [M + |
| ple | Structure | Compound Name | CD₃OD) δ | H]⁺ |
| 33 | 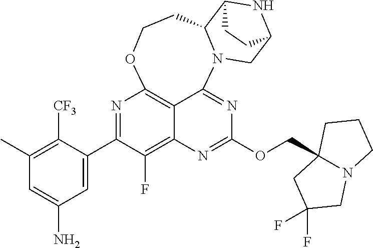 [SMILES: Cc1cc(N)cc(-c2nc3c4c(nc(OC[C@@]56CCCN5CC(F)(F)C6)nc4c2F)N2C[C@H]4CC[C@H](N4)[C@H]2CCO3)c1C(F)(F)F] | 3-((6aR,7S,10R)-13-(((S)-2,2- difluorotetrahydro-1H-pyrrolizin- 7a(5H)-yl)methoxy)-1-fluoro- 5,6,6a,7,8,9,10,11-octahydro-4- oxa-3,11a,12,14,15-pentaaza-7,10- methanocyclohepta[4,5]cycloocta [1,2,3-de]naphthalen-2-yl)-5- methyl-4-(trifluoromethyl)aniline | 6.69 (s, 1H), 6.48-6.41 (m, 1H), 4.67-3.56 (m, 13H), 2.88-2.63(m, 3H), 2.43-1.98 (m, 12H). | 650.5 |
| 34 | 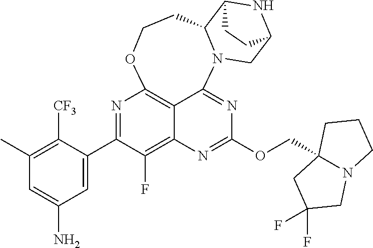 [SMILES: Cc1cc(N)cc(-c2nc3c4c(nc(OC[C@]56CCCN5CC(F)(F)C6)nc4c2F)N2C[C@H]4CC[C@H](N4)[C@H]2CCO3)c1C(F)(F)F] | 3-((6aR,7S,10R)-13-(((R)-2,2- difluorotetrahydro-1H-pyrrolizin- 7a(5H)-yl)methoxy)-1-fluoro- 5,6,6a,7,8,9,10,11-octahydro-4- oxa-3,11a,12,14,15-pentaaza-7,10- methanocyclohepta[4,5]cycloocta [1,2,3-de]naphthalen-2-yl)-5- methyl-4-(trifluoromethyl)aniline | 6.69 (s, 1H), 6.49-6.40 (m, 1H), 4.35-4.26 (m, 4H), 3.97-3.78 (m, 3H), 3.48-3.40 (m, 3H), 3.21-3.11 (m, 2H), 2.89-2.83 (m, 1H), 2.65-2.53 (m, 1H), 2.42-2.28 (m, 5H), 2.18-2.11 (m, 3H), 2.02-1.88 (m, 6H). | 650.5 |
| 35 | 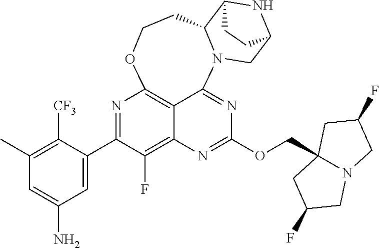 [SMILES: Cc1cc(N)cc(-c2nc3c4c(nc(OC[C@]56C[C@H](F)CN5C[C@H](F)C6)nc4c2F)N2C[C@H]4CC[C@H](N4)[C@H]2CCO3)c1C(F)(F)F] | 3-((6aR,7S,10R)-13-(((2R,6S,7ar)- 2,6-difluorotetrahydro-1H- pyrrolizin-7a(5H)-yl)methoxy)-1- fluoro-5,6,6a,7,8,9,10,11- octahydro-4-oxa-3,11a,12,14,15- pentaaza-7,10- methanocyclohepta[4,5]cycloocta [1,2,3-de]naphthalen-2-yl)-5- methyl-4-(trifluoromethyl)aniline | 6.68 (s, 1H), 6.46-6.43 (m, 1H), 5.44-5.34 (m, 2H), 4.46-4.26 (m, 5H), 4.10-3.97 (m, 2H), 3.65 (s, 1H), 3.57-3.53 (m, 1H), 3.48-3.42 (m, 2H), 2.98-2.89 (m, 2H), 2.65-2.55 (m, 2H), 2.43-2.42 (m, 3H), 2.10-1.95 (m, 5H), 1.83-1.78 (m, 3H). | 650.5 |
| 36 | 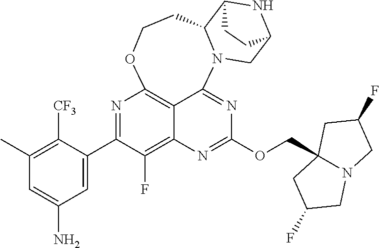 | 3-((6aR,7S,10R)-13-(((2R,6R)- 2,6-difluorotetrahydro-1H- pyrrolizin-7a(5H)-yl)methoxy)-1- fluoro-5,6,6a,7,8,9,10,11- octahydro-4-oxa-3,11a,12,14,15- pentaaza-7,10- methanocyclohepta[4,5]cycloocta [1,2,3-de]naphthalen-2-yl)-5- methyl-4-(trifluoromethyl)aniline | 6.69 (s, 1H), 6.44 (s, 1H), 5.40-5.27 (m, 2H), 4.48-4.31 (m, 4H), 4.13-3.78 (m, 4H), 3.56-3.50 (m, 1H), 3.33-3.32 (m, 1H), 3.26-3.16 (m, 3H), 2.52-2.11 (m, 10H), 2.03-1.90 (m, 3H). | 650.5 |

The following Examples 37-38 were prepared in similar fashion as Example 01 by replacing (2,2-difluorotetrahydro-1H-pyrrolizin-7a(5H)-ylmethanol with the corresponding alkyl alcohols.

| | | | | MS |
|---|---|---|---|---|
| | | | | (ESI, |
| Ex- | | | | m/e) |
| am- | | | ¹H NMR (500 MHz, | [M + |
| ple | Structure | Compound Name | CD₃OD) δ | H]⁺ |
| 37 | 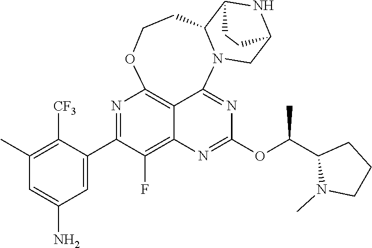 [SMILES: Cc1cc(N)cc(-c2nc3c4c(nc(O[C@@H](C)[C@@H]5CCCN5C)nc4c2F)N2C[C@H]4CC[C@H](N4)[C@H]2CCO3)c1C(F)(F)F] | 3-((6aR,7S,10R)-1-fluoro-13-((S)- 1-((S)-1-methylpyrrolidin-2- yl)ethoxy)-5,6,6a,7,8,9,10,11- octahydro-4-oxa-3,11a,12,14,15- pentaaza-7,10- methanocyclohepta[4,5]cycloocta [1,2,3-de]naphthalen-2-yl)-5- methyl-4-(trifluoromethyl)aniline | 6.69 (s, 1H), 6.46- 6.40(m, 1H), 5.30-5.28 (m, 1H), 4.37-3.96(m, 5H), 3.75-3.60 (m, 2H), 3.33-3.25 (m, 3H), 2.90-2.71 (m, 3H), 2.42 (s, 3H), 2.24-1.86 (m, 10H), 1.48-1.47 (m, 3H) | 602.5 |
| 38 | 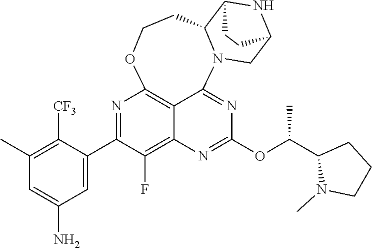 [SMILES: Cc1cc(N)cc(-c2nc3c4c(nc(O[C@H](C)[C@@H]5CCCN5C)nc4c2F)N2C[C@H]4CC[C@H](N4)[C@H]2CCO3)c1C(F)(F)F] | 3-((6aR,7S,10R)-1-fluoro-13-((R)- 1-((S)-1-methylpyrrolidin-2- yl)ethoxy)-5,6,6a,7,8,9,10,11- octahydro-4-oxa-3,11a,12,14,15- pentaaza-7,10- methanocyclohepta[4,5]cycloocta [1,2,3-de]naphthalen-2-yl)-5- methyl-4-(trifluoromethyl)aniline | 6.69 (s, 1H), 6.47-6.41 (m, 1H), 5.71-5.65 (m, 1H), 4.40-3.99 (m, 2H), 3.49-3.41 (m, 1H), 2.94-2.85 (m, 4H), 2.42 (s, 3H), 2.24-1.87 (m, 10H), 1.47-1.46 (m, 3H) | 602.5 |

Examples 39-41: The following examples were prepared in a manner similar to that in Example 07 by replacing (3R,4R)-3-aminotetrahydro-2H-pyran-4-ol with the corresponding amino alcohols.

| | | | | MS |
|---|---|---|---|---|
| | | | | (ESI, |
| Ex- | | | | m/e) |
| am- | | | 1H NMR (500 MHz, | [M + |
| ple | Structure | Compound Name | CD₃OD) δ | H]⁺ |
| 39 | 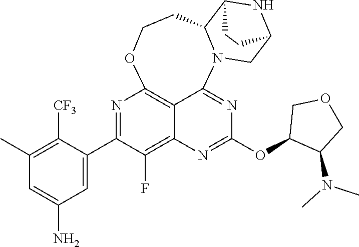 | (3R,4R)-4-(((6aR,7S,10R)-2-(5- amino-3-methyl-2- (trifluoromethyl)phenyl)-1-fluoro- 5,6,6a,7,8,9,10,11-octahydro-4- oxa-3,11a,12,14,15-pentaaza-7,10- methanocyclohepta[4,5]cycloocta [1,2,3-de]naphthalen-13-yl)oxy)- N,N-dimethyltetrahydrofuran-3- amine | 6.70 (s, 1H), 6.44 (s, 1H), 5.81-5.80 (m, 1H), 4.31-3.92 (m,11H), 3.17-3.16 (m, 1H), 2.42-1.91 (m, 15H). | 604.5 |
| 40 | 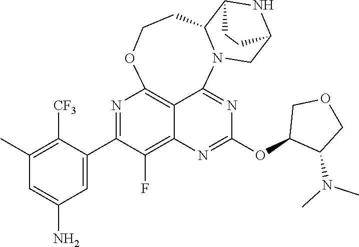 | (3S,4R)-4-(((6aR,7S,10R)-2-(5- amino-3-methyl-2- (trifluoromethyl)phenyl)-1-fluoro- 5,6,6a,7,8,9,10,11-octahydro-4- oxa-3,11a,12,14,15-pentaaza-7,10- methanocyclohepta[4,5]cycloocta [1,2,3-de]naphthalen-13-yl)oxy)- N,N-dimethyltetrahydrofuran-3- amine | 6.70 (s, 1H), 6.44 (s, 1H), 5.56-5.55 (m, 1H), 4.30-3.72 (m,11H), 3.23-3.22 (m, 1H), 2.41-2.39 (m, 9H), 2.25-1.93 (m, 6H). | 604.5 |
| 41 | 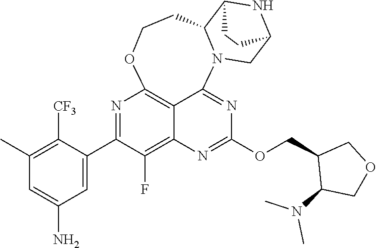 [SMILES: Cc1cc(N)cc(-c2nc3c4c(nc(OC[C@H]5COC[C@H]5N(C)C)nc4c2F)N2C[C@H]4CC[C@H](N4)[C@H]2CCO3)c1C(F)(F)F] | (3S,4R)-4-((((6aR,7S,10R)-2-(5- amino-3-methyl-2- (trifluoromethyl)phenyl)-1-fluoro- 5,6,6a,7,8,9,10,11-octahydro-4- oxa-3,11a,12,14,15-pentaaza-7,10- methanocyclohepta[4,5]cycloocta [1,2,3-de]naphthalen-13- yl)oxy)methyl)-N,N- dimethyltetrahydrofuran-3-amine | (500 MHz, DMSO) δ 6.60 (s, 1H), 6.35 (s, 1H), 5.87 (s, 2H), 4.67- 3.53 (m, 13H), 2.78- 2.76 (m, 2H), 2.36 (s, 3H), 2.20 (m, 6H), 1.99- 1.55 (m, 6H). | 618.5 |

Examples 42-43: The following examples were prepared by further separation of Example 15 as a mixture of two isomers via chiral Prep-HPLC (Column: i-Cellulose-5, 21.2 mm×250 mm, 5 μm; Mobile Phase A: n-Hexane, Mobile Phase B: EtOH (with 0.2% 2M NH₃ in MeOH); Flow Rate: 18 mL/min; Temperature: 25° C.; Gradient: 50% B in 18 min).

| | | | | MS |
|---|---|---|---|---|
| | | | | (ESI, |
| Ex- | | | | m/e) |
| am- | | | 1H NMR (500 MHz, | [M + |
| ple | Structure | Compound Name | CD₃OD) δ | H]⁺ |
| 42 | 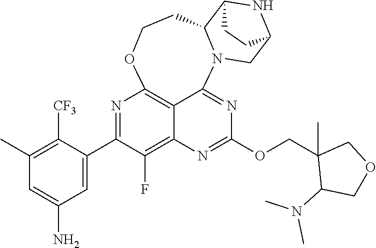 [SMILES: Cc1cc(N)cc(-c2nc3c4c(nc(OCC5(C)COCC5N(C)C)nc4c2F)N2C[C@H]4CC[C@H](N4)[C@H]2CCO3)c1C(F)(F)F] (isomer 1) | 4-((((6aR,7S,10R)-2-(5-amino-3- methyl-2-(trifluoromethyl)phenyl)- 1-fluoro-5,6,6a,7,8,9,10,11- octahydro-4-oxa-3,11a,12,14,15- pentaaza-7,10- methanocyclohepta[4,5]cycloocta [1,2,3-de]naphthalen-13- yl)oxy)methyl)-N,N,4- trimethyltetrahydrofuran-3-amine | 6.70 (s, 1H), 6.45-6.44 (m, 1H), 4.50-3.63 (m, 13H), 2.98-2.97 (m, 1H), 2.42 (s, 3H), 2.37 (s, 6H), 2.25-1.91 (m, 6H), 1.29 (s, 3H). | 632.5 |
| 43 | 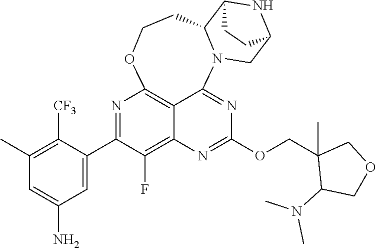 [SMILES: Cc1cc(N)cc(-c2nc3c4c(nc(OCC5(C)COCC5N(C)C)nc4c2F)N2C[C@H]4CC[C@H](N4)[C@H]2CCO3)c1C(F)(F)F] (isomer 2) | 4-((((6aR,7S,10R)-2-(5-amino-3- methyl-2-(trifluoromethyl)phenyl)- 1 -fluoro-5,6,6a,7,8,9,10,11- octahydro-4-oxa-3,11a,12,14,15- pentaaza-7,10- methanocyclohepta[4,5]cycloocta [1,2,3-de]naphthalen-13- yl)oxy)methyl)-N,N,4- trimethyltetrahydrofuran-3-amine | 6.70 (s, 1H), 6.45-6.44 (m, 1H), 4.523.63 (m, 13H), 2.99-2.97 (m, 1H), 2.42 (s, 3H), 2.37 (s, 6H), 2.25-1.91 (m, 6H), 1.28 (s, 3H). | 632.5 |

Examples 44-45: The following examples were prepared in a manner similar to that in Example 18 by replacing the corresponding alkyl alcohol as mixture of isomers with the alkyl alcohols as separated single enantiomer.

| | | | | MS |
|---|---|---|---|---|
| | | | | (ESI, |
| | | | 1H NMR (500 MHz, | m/e) |
| Example | Structure | Compound Name | CD₃OD) δ | [M + H]⁺ |
| 44 | 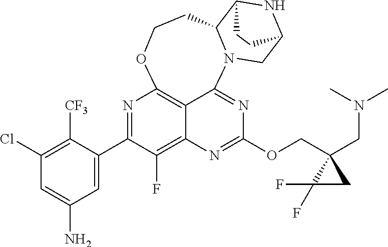 [SMILES: CN(C)C[C@]1(COc2nc3c4c(nc(-c5cc(N)cc(Cl)c5C(F)(F)F)c(F)c4n2)OCC[C@@H]2[C@@H]4CC[C@H](CN32)N4)CC1(F)F] | 3-chloro-5-((6aR,7S,10R)-13- (((S)-1- ((dimethylamino)methyl)-2,2- difluorocyclopropyl)methoxy)-1- fluoro-5,6,6a,7,8,9,10,11- octahydro-4-oxa-3,11a,12,14,15- pentaaza-7,10- methanocyclohepta[4,5]cycloocta [1,2,3-de]naphthalen-2-yl)-4- (trifluoromethyl)aniline | 6.88 (s, 1H), 6.55-6.48 (m, 1H), 4.53-3.44 (m, 9H), 2.81-2.46 (m, 2H), 2.27 (s, 6H), 2.22-1.41 (m, 8H). | 658.4 |
| 45 | 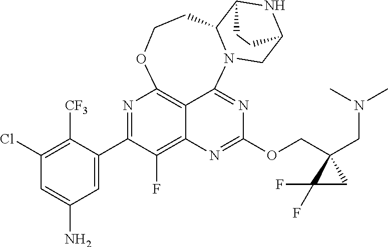 [SMILES: CN(C)C[C@@]1(COc2nc3c4c(nc(-c5cc(N)cc(Cl)c5C(F)(F)F)c(F)c4n2)OCC[C@@H]2[C@@H]4CC[C@H](CN32)N4)CC1(F)F] | 3-chloro-5-((6aR,7S,10R)-13- (((R)-1- ((dimethylamino)methyl)-2,2- difluorocyclopropyl)methoxy)-1- fluoro-5,6,6a,7,8,9,10,11- octahydro-4-oxa-3,11a,12,14,15- pentaaza-7,10- methanocyclohepta[4,5] cycloocta[1,2,3-de] naphthalen-2-yl)-4- (trifluoromethyl)aniline | 6.88 (s, 1H), 6.55-6.48 (m,1H), 4.58-3.54 (m, 9H), 2.84-2.42 (m, 2H), 2.27 (s, 6H), 2.08-1.36 (m, 8H). | 658.4 |

Example 46-56: The following examples were prepared in a manner similar to that in Example 16, 17 by replacing the corresponding substitution or coupling partners.

| | | | | MS |
|---|---|---|---|---|
| | | | | (ESI, |
| | | | | m/e) |
| | | | 1H NMR (500 MHz, | [M + |
| Example | Structure | Compound Name | CD₃OD) δ | H]⁺ |
| 46 | 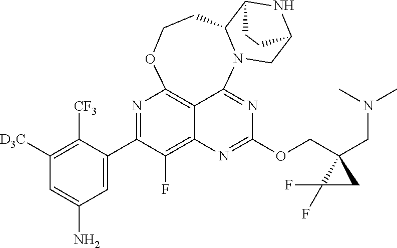 [SMILES: [2H]C([2H])([2H])c1cc(N)cc(-c2nc3c4c(nc(OC[C@@]5(CN(C)C)CC5(F)F)nc4c2F)N2C[C@H]4CC[C@H](N4)[C@H]2CCO3)c1C(F)(F)F] | 3-((6aR,7S,10R)-13-(((S)-1- ((dimethylamino)methyl)-2,2- difluorocyclopropyl)methoxy)-1- fluoro-5,6,6a,7,8,9,10,11- octahydro-4-oxa-3,11a,12,14,15- pentaaza-7,10- methanocyclohepta[4,5]cycloocta [1,2,3-de]naphthalen-2-yl)-5- (methyl-d3)-4- (trifluoromethyl)aniline | 6.68 (s, 1H), 6.46-6.41 (m, 1H), 4.62-4.54 (m, 2H), 4.38-4.37 (m, 3H), 4.09-4.01 (m, 2H), 3.72- 3.69 (m, 1H), 3.59-3.55 (m, 1H), 2.83-2.80 (m, 1H), 2.48-2.45 (m, 1H), 2.27 (s, 6H), 2.09-1.79 (m, 6H), 1.70-1.65 (m, 1H), 1.42-1.37 (m, 1H). | 641.5 |
| 47 | 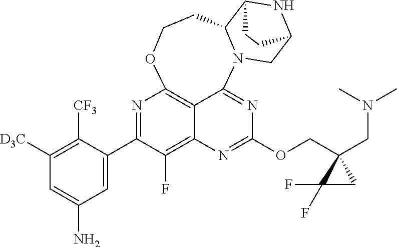 [SMILES: [2H]C([2H])([2H])c1cc(N)cc(-c2nc3c4c(nc(OC[C@]5(CN(C)C)CC5(F)F)nc4c2F)N2C[C@H]4CC[C@H](N4)[C@H]2CCO3)c1C(F)(F)F] | 3-((6aR,7S,10R)-13-(((R)-1- ((dimethylamino)methyl)-2,2- difluorocyclopropyl)methoxy)-1- fluoro-5,6,6a,7,8,9,10,11- octahydro-4-oxa-3,11a,12,14,15- pentaaza-7,10- methanocyclohepta[4,5]cycloocta [1,2,3-de]naphthalen-2-yl)-5- (methyl-d3)-4- (trifluoromethyl)aniline | 6.68 (s, 1H), 6.47-6.42 (m, 1H), 4.58 (s, 2H), 4.37-4.27 (m, 3H), 4.09- 3.99 (m, 2H), 3.68 (s, 1H), 3.58-3.54 (m, 1H), 2.86-2.83 (m, 1H), 2.45- 2.43 (m, 1H), 2.27 (s, 6H), 2.10-1.79 (m, 6H), 1.70-1.65 (m, 1H), 1.42- 1.37 (m, 1H). | 641.5 |
| 48 | 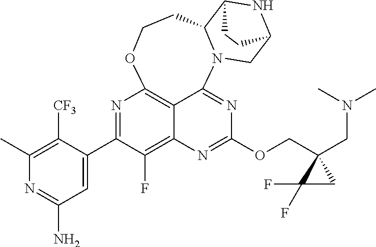 | 4-((6aR,7S,10R)-13-(((R)-1- ((dimethylamino)methyl)-2,2- difluorocyclopropyl)methoxy)-1- fluoro-5,6,6a,7,8,9,10,11- octahydro-4-oxa-3,11a,12,14,15- pentaaza-7,10- methanocyclohepta[4,5]cycloocta [1,2,3-de]naphthalen-2-yl)-6- methyl-5- (trifluoromethyl)pyridin-2-amine | 6.38 (s, 1H), 4.59-3.69 (m, 9H), 2.91-2.89 (m, 1H), 2.54-2.53 (m, 4H), 2.32 (s, 6H), 2.10- 1.40 (m, 8H). | 639.5 |
| 49 | 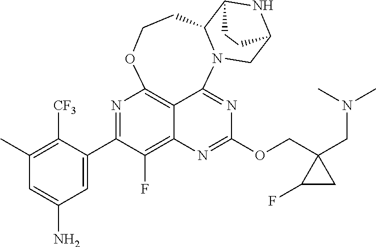 | 3-((6aR,7S,10R)-13-((1- ((dimethylamino)methyl)-2- fluorocyclopropyl)methoxy)-1- fluoro-5,6,6a,7,8,9,10,11- octahydro-4-oxa-3,11a,12,14,15- pentaaza-7,10- methanocyclohepta[4,5]cycloocta [1,2,3-de]naphthalen-2-yl)-5- methyl-4-(trifluoromethyl)aniline | 6.69 (s, 1H), 6.48-6.47 (m,1H), 4.82-3.77 (m, 10H), 3.16-2.68 (m, 8H), 2.42 (s, 3H), 2.18- 1.93 (m, 6H), 1.29-1.11 (m, 2H). | 620.5 |
| | (isomer 1 and 2, as a | | | |
| | mixture of two | | | |
| | isomers) | | | |
| 50 | 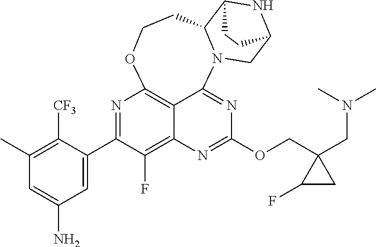 | 3-((6aR,7S,10R)-13-((1- ((dimethylamino)methyl)-2- fluorocyclopropyl)methoxy)-1- fluoro-5,6,6a,7,8,9,10,11- octahydro-4-oxa-3,11a,12,14,15- pentaaza-7,10- methanocyclohepta[4,5]cycloocta [1,2,3-de]naphthalen-2-yl)-5- methyl-4-(trifluoromethyl)aniline | 6.70 (s, 1H), 6.48-6.47 (m,1H), 4.82-3.79 (m, 10H), 3.16-2.68 (m, 8H), 2.42 (s, 3H), 2.18- 1.93 (m, 6H), 1.29-1.09 (m, 2H). | 620.5 |
| | (isomer 3) | | | |
| 51 | 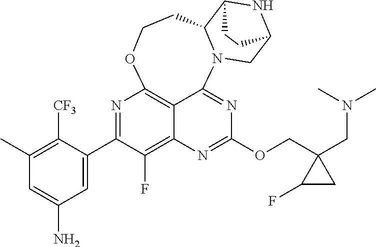 | 3-((6aR,7S,10R)-13-((1- ((dimethylamino)methyl)-2- fluorocyclopropyl)methoxy)-1- fluoro-5,6,6a,7,8,9,10,11- octahydro-4-oxa-3,11a,12,14,15- pentaaza-7,10- methanocyclohepta[4,5]cycloocta [1,2,3-de]naphthalen-2-yl)-5- methyl-4-(trifluoromethyl)aniline | 6.69 (s, 1H), 6.48-6.47 (m,1H), 4.82-3.72 (m, 10H), 3.16-2.68 (m, 8H), 2.42 (s, 3H), 2.18- 1.93 (m, 6H), 1.29-1.09 (m, 2H). | 620.5 |
| | (isomer 4) | | | |
| 52 | 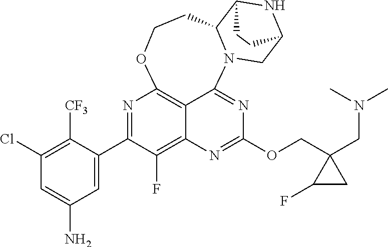 [SMILES: CN(C)CC1(COc2nc3c4c(nc(-c5cc(N)cc(Cl)c5C(F)(F)F)c(F)c4n2)OCC[C@@H]2[C@@H]4CC[C@H](CN32)N4)CC1F] | 3-chloro-5-((6aR,7S,10R)-13-((1- ((dimethylamino)methyl)-2- fluorocyclopropyl)methoxy)-1- fluoro-5,6,6a,7,8,9,10,11- octahydro-4-oxa-3,11a,12,14,15- pentaaza-7,10- methanocyclohepta[4,5]cycloocta [1,2,3-de]naphthalen-2-yl)-4- (trifluoromethyl)aniline | 6.88 (s, 1H), 6.54-6.47 (m, 1H), 4.69-3.44 (m, 10H), 2.80-2.32 (m, 8H), 2.03-1.85 (m, 6H), 1.29-1.18 (m, 2H). | 640.5 |
| | (isomer 1) | | | |
| 53 | 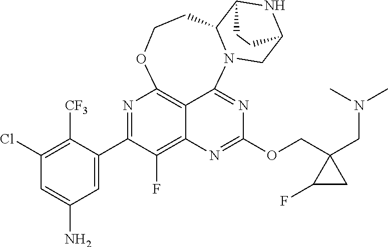 [SMILES: CN(C)CC1(COc2nc3c4c(nc(-c5cc(N)cc(Cl)c5C(F)(F)F)c(F)c4n2)OCC[C@@H]2[C@@H]4CC[C@H](CN32)N4)CC1F] | 3-chloro-5-((6aR,7S,10R)-13-((1- ((dimethylamino)methyl)-2- fluorocyclopropyl)methoxy)-1- fluoro-5,6,6a,7,8,9,10,11- octahydro-4-oxa-3,11a,12,14,15- pentaaza-7,10- methanocyclohepta[4,5]cycloocta [1,2,3-de]naphthalen-2-yl)-4- (trifluoromethyl)aniline | 6.88 (s, 1H), 6.55-6.47 (m, 1H), 4.71-3.44 (m, 10H), 2.92-2.32 (m, 8H), 2.09-1.85 (m, 6H), 1.17-1.11 (m, 2H). | 640.5 |
| | (isomer 2) | | | |
| 54 | 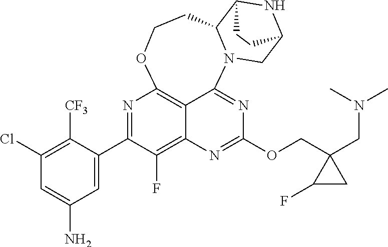 [SMILES: CN(C)CC1(COc2nc3c4c(nc(-c5cc(N)cc(Cl)c5C(F)(F)F)c(F)c4n2)OCC[C@@H]2[C@@H]4CC[C@H](CN32)N4)CC1F] | 3-chloro-5-((6aR,7S,10R)-13-((1- ((dimethylamino)methyl)-2- fluorocyclopropyl)methoxy)-1- fluoro-5,6,6a,7,8,9,10,11- octahydro-4-oxa-3,11a,12,14,15- pentaaza-7,10- methanocyclohepta[4,5]cycloocta [1,2,3-de]naphthalen-2-yl)-4- (trifluoromethyl)aniline | 6.88 (s, 1H), 6.55-6.48 (m, 1H), 4.62-3.44 (m, 10H), 2.40-2.30 (m, 8H), 2.09-1.61 (m, 6H), 1.29-1.18 (m, 2H). | 640.5 |
| | (isomer 3) | | | |
| 55 | 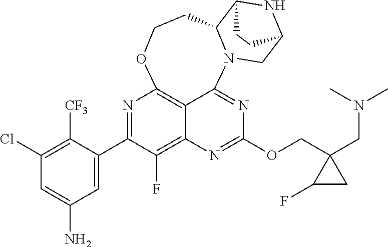 [SMILES: CN(C)CC1(COc2nc3c4c(nc(-c5cc(N)cc(Cl)c5C(F)(F)F)c(F)c4n2)OCC[C@@H]2[C@@H]4CC[C@H](CN32)N4)CC1F] | 3-chloro-5-((6aR,7S,10R)-13-((1- ((dimethylamino)methyl)-2- fluorocyclopropyl)methoxy)-1- fluoro-5,6,6a,7,8,9,10,11- octahydro-4-oxa-3,11a,12,14,15- pentaaza-7,10- methanocyclohepta[4,5]cycloocta [1,2,3-de]naphthalen-2-yl)-4- (trifluoromethyl)aniline | 6.88 (s, 1H), 6.55-6.48 (m, 1H), 4.66-3.44 (m, 10H), 2.42-2.31 (m, 8H), 2.10-1.86 (m, 6H), 1.26-1.11 (m, 2H). | 640.5 |
| | (isomer 4) | | | |
| 56 | 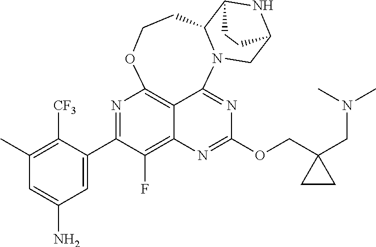 | 3-((6aR,7S,10R)-13-((1- ((dimethylamino)methyl) cyclopropyl)methoxy)-1-fluoro- 5,6,6a,7,8,9,10,11-octahydro-4- oxa-3,11a,12,14,15-pentaaza- 7,10- methanocyclohepta[4,5]cycloocta [1,2,3-de]naphthalen-2-yl)-5- methyl-4-(trifluoromethyl)aniline | 6.68 (s, 1H), 6.44-6.43 (m,1H), 4.35-3.59 (m, 9H), 2.46-2.42 (m, 5H), 2.30 (s, 3H), 2.08- 1.79 (m, 6H), 0.72- 0.53 (m, 4H). | 602.5 |

Examples 57-58: The following examples were prepared by further separation of Example 19 via chiral Prep-HPLC (Column: i-Cellulose-5, 21.2 mm×250 mm, 5 μm; Mobile Phase A: n-Hexane, Mobile Phase B: EtOH (with 0.2% 2M NH₃ in MeOH); Flow Rate: 18 mL/min; Temperature: 25° C.; Gradient: 50% B in 18 min).

| | | | | MS |
|---|---|---|---|---|
| | | | | (ESI, |
| | | | | m/e) |
| | | | 1H NMR (500 MHz, | [M + |
| Example | Structure | Compound Name | CD₃OD) δ | H]⁺ |
| 57 | 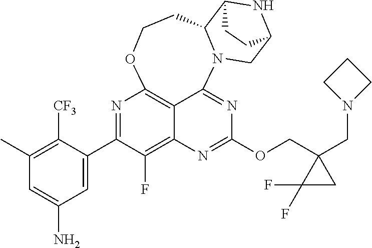 [SMILES: Cc1cc(N)cc(-c2nc3c4c(nc(OCC5(CN6CCC6)CC5(F)F)nc4c2F)N2C[C@H]4CC[C@H](N4)[C@H]2CCO3)c1C(F)(F)F] | 3-((6aR,7S,10R)-13-((1- (azetidin-1-ylmethyl)-2,2- difluorocyclopropyl)methoxy)- 1-fluoro-5,6,6a,7,8,9,10,11- octahydro-4-oxa-3,11a,12,14,15- pentaaza-7,10- methanocyclohepta [4,5]cycloocta [1,2,3-de]naphthalen-2-yl)-5- methyl-4- (trifluoromethyl)aniline | 6.70 (s, 1H), 6.46-6.40 (m, 1H), 4.50-4.40 (m, 12H), 3.68-3.56 (m, 3H), 2.42 (s, 3H), 2.05-1.02 (m, 12H). | 650.5 |
| | (isomer 1) | | | |
| 58 | 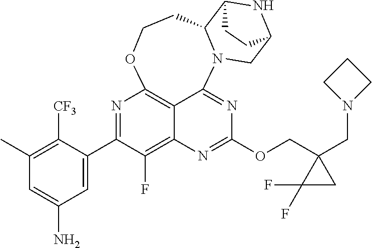 [SMILES: Cc1cc(N)cc(-c2nc3c4c(nc(OCC5(CN6CCC6)CC5(F)F)nc4c2F)N2C[C@H]4CC[C@H](N4)[C@H]2CCO3)c1C(F)(F)F] | 3-((6aR,7S,10R)-13-((1- (azetidin-1-ylmethyl)-2,2- difluorocyclopropyl)methoxy)- 1-fluoro-5,6,6a,7,8,9,10,11- octahydro-4-oxa-3,11a,12, 14,15-pentaaza-7,10- methanocyclohepta [4,5]cycloocta [1,2,3-de]naphthalen-2-yl)-5- methyl-4- (trifluoromethyl)aniline | 6.71 (s, 1H), 6.46-6.40 (m, 1H), 4.74-4.20 (m, 11H), 3.68-3.59 (m, 3H), 3.22-3.20 (m, 2H), 2.42 (s, 3H), 2.19-1.02 (m, 12H). | 650.5 |
| | (isomer 2) | | | |

### Example 59-62: 3-((6aR,7S,10R)-13-((1-(1-(dimethylamino)ethyl)-2,2-difluorocyclopropyl)methoxy)-1-fluoro-5,6,6a,7,8,9,10,11-octahydro-4-oxa-3,11a,12,14,15-pentaaza-7,10-methanocyclohepta[4,5]cycloocta[1,2,3-de]naphthalen-2-yl)-5-methyl-4-(trifluoromethyl)aniline

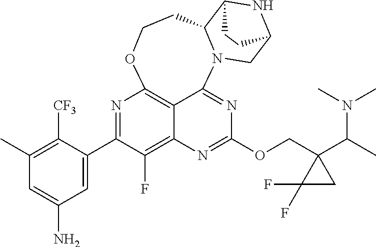

### Synthetic Route

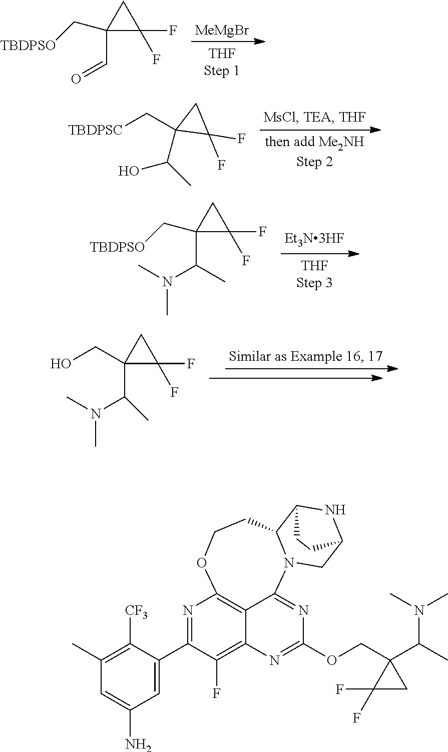

### Step 1: 1-(1-(((tert-butyldiphenylsilyl)oxy)methyl)-2,2-difluorocyclopropyl)ethan-1-ol

To a 100-mL round-bottom flask was added 1-(((tert-butyldiphenylsilyl)oxy)methyl)-2,2-difluorocyclopropane-1-carbaldehyde (374 mg, 1.0 mmol), MeMgBr (3M, 0.5 mL, 1.5 mmol) and THF (10 mL). The reaction mixture was stirred at rt for 2 h, which was then concentrated in vacuo. The crude was purified by column chromatography (EA/PE=0-20%), affording the title product (350 mg, 90% yield) as a colorless oil.

### Step 2: 1-(1-(((tert-butyldiphenylsilyl)oxy)methyl)-2,2-difluorocyclopropyl)-N,N-dimethylethan-1-amine

To a 50-mL round-bottom flask were added 1-(1-(((tert-butyldiphenylsilyl)oxy)methyl)-2,2-difluorocyclopropyl)ethan-1-ol (350 mg, 0.90 mmol), THF (6 mL), Et₃N (181.8 mg, 1.8 mmol) and MsCl (205 mg, 1.8 mmol). The reaction mixture was stirred at rt for 30 min, followed by addition of dimethylamine (2M in THF, 20 mL, 40 mmol) in THF (6 mL) at rt. The resulting mixture was stirred at 65° C. for 32 h in the sealed tube. After completion. The reaction was then quenched by addition of sat. NH4Cl (20 mL). The mixture was extracted by EtOAc (20 mL) for 3 times. The organic phase was combined, dried with Na2SO4, and filtered through Celite. The mixture was concentrated in vacuo. The resulting crude was purified by column chromatography (EA/PE=0-30%), affording the title compound (300 mg, 80% yield) as a colorless oil.

### Step 3: (1-(1-(dimethylamino)ethyl)-2,2-difluorocyclopropyl)methanol

To a 100 mL round-bottom flask were added 1-(1-(((tert-butyldiphenylsilyl)oxy)methyl)-2,2-difluorocyclopropyl)-N,N-dimethylethan-1-amine (300 mg, 0.72 mmol), Et₃N·3HF (116 mg, 0.72 mol), and THF (10 mL). The reaction mixture was stirred at rt for 2 h. After the reaction is complete as indicated by LCMS. The resulting mixture was concentrated in vacuo. The crude was purified by column chromatography (MeOH/DCM=0-10%), affording the title compound (50 mg, 39% yield) as a colorless oil.

Steps 4-6: 3-((6aR,7S,10R)-13-((1-(1-(dimethylamino)ethyl)-2,2-difluorocyclopropyl)methoxy)-1-fluoro-5,6,6a,7,8,9,10,11-octahydro-4-oxa-3,11a,12,14,15-pentaaza-7,10-methanocyclohepta[4,5]cycloocta[1,2,3-de]naphthalen-2-yl)-5-methyl-4-(trifluoromethyl)aniline as a mixture of 4 isomers were prepared in similar fashion as Example 01 by replacing (2,2-difluorotetrahydro-1H-pyrrolizin-7a(5H)-yl)methanol with (1-(1-(dimethylamino)ethyl)-2,2-difluorocyclopropyl)methanol.

Step 7: The aforementioned mixture of 4 isomers (30 mg) was separated by chiral Prep-HPLC (Column: i-Cellulose-5, 21.2 mm×250 mm, 5 μm; Mobile Phase A: n-Hexane, Mobile Phase B: EtOH (with 0.2% 2M NH₃ in MeOH); Flow Rate: 18 mL/min; Temperature: 25° C.; Gradient: 50% B in 18 min; Retention time—isomer 1: 9.0 min; Retention time—isomer 2: 12.0 min) to give 4 eluting products as Example 59-62:

Example 59 (isomer 1): 1.2 mg isolated. ¹H NMR (500 MHz, DMSO-d₈) δ 6.59 (s, 1H), 6.35 (s, 1H), 5.87 (s, 2H), 4.71-3.43 (m, 9H), 2.36 (s, 3H), 2.17 (s 6H), 1.99-1.40 (m, 9H), 1.20-1.19 (m, 3H). MS (ESI, m/e) [M+H]⁺ 652.5.

Example 60 (isomer 2): 2.0 mg isolated. ¹H NMR (500 MHz, DMSO-d₈) δ 6.59 (s, 1H), 6.36 (s, 1H), 5.87 (s, 2H), 4.72-3.43 (m, 9H), 2.36 (s, 3H), 2.17 (s 6H), 1.99-1.50 (m, 9H), 1.21-1.19 (m, 3H). MS (ESI, m/e) [M+H]⁺ 652.5.

Example 61 (isomer 3): 6.1 mg isolated. ¹H NMR (500 MHz, DMSO-d₈) δ 6.59 (s, 1H), 6.36 (s, 1H), 5.87 (s, 2H), 4.72-3.45 (m, 9H), 2.36 (s, 3H), 2.17 (s 6H), 1.99-1.45 (m, 9H), 1.21-1.19 (m, 3H). MS (ESI, m/e) [M+H]⁺ 652.5.

Example 62 (isomer 4): 1.2 mg isolated. ¹H NMR (500 MHz, DMSO-ds) δ 6.59 (s, 1H), 6.36 (s, 1H), 5.87 (s, 2H), 4.72-3.43 (m, 9H), 2.36 (m, 3H), 2.17 (s, 6H), 1.99-1.5 (m, 9H), 1.21-1.19 (m, 3H). MS (ESI, m/e) [M+H]⁺ 652.5.

### Example 63: 3-((6aR,7S,10R)-1-fluoro-13-((1-(1-methylpyrrolidin-2-yl)cyclopropyl)methoxy)-5,6,6a,7,8,9,10,11-octahydro-4-oxa-3,11a,12,14,15-pentaaza-7,10-methanocyclohepta[4,5]cycloocta[1,2,3-de]naphthalen-2-yl)-5-methyl-4-(trifluoromethyl)aniline

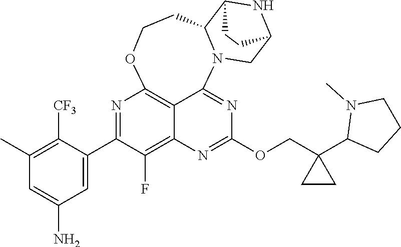 [SMILES: Cc1cc(N)cc(-c2nc3c4c(nc(OCC5(C6CCCN6C)CC5)nc4c2F)N2C[C@H]4CC[C@H](N4)[C@H]2CCO3)c1C(F)(F)F]

### Synthetic Route

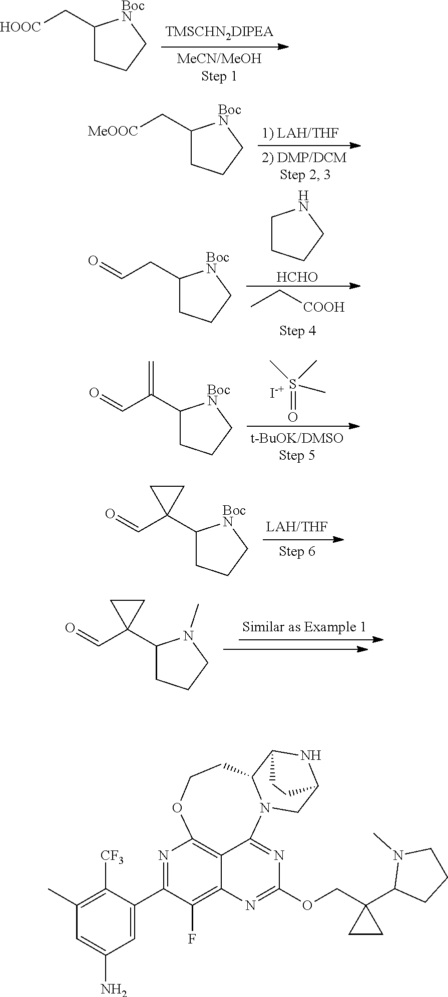

### Step 1: tert-butyl 2-(2-methoxy-2-oxoethyl)pyrrolidine-1-carboxylate

To a mixture of 2-(1-(tert-butoxycarbonyl)pyrrolidin-2-yl)acetic acid (4.6 g, 20 mmol) and DIPEA (3.56 mL, 21.4 mmol) in MeOH (2.64 mL) and MeCN (80 mL) was added diazomethyl(trimethyl)silane (2 M, 18 mL, 36 mmol). The mixture was stirred at room temperature for 20 hours. The reaction was then quenched by addition of saturated NaHCO₃ (50 mL). The resulting mixture was extracted by EtOAc (50 mL) for 3 times. The organic phase was combined, dried over Na₂SO₄, and concentrated in vacuo to give the crude title product (4 g).

### Step 2: tert-butyl 2-(2-hydroxyethyl)pyrrolidine-1-carboxylate

To a solution of tert-butyl 2-(2-methoxy-2-oxoethyl)pyrrolidine-1-carboxylate (4 g) in THF (50 mL) was added LiAlH₄ (912 mg, 24 mmol). The resulting mixture was stirred at rt for 1 hour. The reaction was then quenched with Na₂SO₄·10H₂O, filtered through Celite, and concentrated in vacuo to give the crude product (4.0 g).

### Step 3: tert-butyl 2-(2-oxoethyl)pyrrolidine-1-carboxylate

To a 250 mL round-bottom flask were added tert-butyl 2-(2-hydroxyethyl)pyrrolidine-1-carboxylate (4.0 g), DMP (17.0 g, 40.0 mmol) and DCM (100 mL) at rt. The reaction mixture was stirred at rt for 2 hours, which was then concentrated in vacuo. The crude was purified by column chromatography (EtOAc/PE=0 to 40%), affording the title product (3.2 g) as a colorless oil.

### Step 4: tert-butyl 2-(3-oxoprop-1-en-2-yl)pyrrolidine-1-carboxylate

To a 100-mL round-bottom flask were added tert-butyl 2-(2-oxoethyl)pyrrolidine-1-carboxylate (1.9 g, 8.9 mmol), pyrrolidine (63 mg, 0.89 mmol), 37% HCHO solution (0.8 mL, 10.7 mmol), propionic acid (65.9 mg, 0.89 mmol) and MeOH (40 mL). The reaction mixture was stirred at 55° C. for 16 hours. After the reaction was complete as indicated by TLC, the reaction mixture was concentrated in vacuo. The crude was purified by column chromatography (EtOAc/PE=0 to 20%), affording the title product (1.4 g, 70% yield) as a colorless oil.

### Step 5: tert-butyl 2-(1-formylcyclopropyl)pyrrolidine-1-carboxylate

To a 50-mL round-bottom flask were added trimethyloxosulphonium iodide (1.76 g, 8.0 mmol), potassium tert-butoxide (896 mg, 8.0 mmol) and DMSO (20 mL). The reaction mixture was stirred at 55° C. for 10 min, followed by addition of tert-butyl 2-(3-oxoprop-1-en-2-yl)pyrrolidine-1-carboxylate (1.2 g, 5.3 mmol) in DMSO (10 mL) at rt. The resulting mixture was stirred at 55° C. for 0.5 h. After the reaction was complete as indicated by TLC, the reaction was quenched by addition of sat. NaHCO₃ (30 mL). The mixture was extracted by EtOAc (20 mL) for 3 times. The organic phase was combined, dried over Na₂SO₄, and filtered through Celite. The filtrate was concentrated in vacuo. The resulting crude was purified by column chromatography (EA/PE=0-30%), affording the title compound (750 mg, 59% yield) as a colorless oil.

### Step 6: (1-(1-methylpyrrolidin-2-yl)cyclopropyl)methanol

To a solution of tert-butyl 2-(1-formylcyclopropyl)pyrrolidine-1-carboxylate (239 mg, 1.0 mmol) in THF (15 mL) was added LiAlH₄ (114 mg, 3 mmol). The resulting mixture was stirred at 65° C. for 16 hours. After the reaction was complete as indicated by TLC, the reaction was quenched with Na₂SO₄·10H₂O, and filtered through Celite. The filtrate was concentrated in vacuo to give the crude product (80 mg).

Steps 7-9: Example 63 as a mixture of two isomers was prepared in similar fashion as Example 01 by replacing (2,2-difluorotetrahydro-1H-pyrrolizin-7a(5H)-yl)methanol with (1-(1-methylpyrrolidin-2-yl)cyclopropyl)methanol. ¹H NMR (500 MHz, CD₃OD) δ 6.68 (s, 1H), 6.46-6.40 (m, 1H), 4.54-3.44 (m, 12H), 2.49-2.35 (m, 6H), 1.986-1.60 (m, 10H), 0.90-0.52 (m, 4H). MS (ESI, m/e) [M+H]⁺ 628.5.

Examples 64-71: The following examples were prepared in a manner similar to that in Example 19 by replacing (2,2-difluorocyclopropane-1,1-diyl)dimethanol with cyclopropane-1,1-diyldimethanol, and replacing azetidine with the corresponding amines.

| | | | | MS |
|---|---|---|---|---|
| | | | | (ESI, |
| | | | | m/e) |
| | | | 1H NMR (500 MHz, | [M + |
| Example | Structure | Compound Name | CD₃OD) δ | H]⁺ |
| 64 | 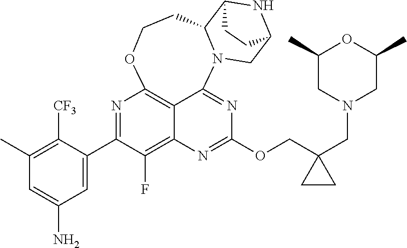 | 3-((6aR,7S,10R)-13-((1-(((2S,6R)- 2,6- dimethylmorpholino)methyl) cyclopropyl)methoxy)-1-fluoro- 5,6,6a,7,8,9,10,11-octahydro-4-oxa- 3,11a,12,14,15-pentaaza-7,10- methanocyclohepta[4,5]cycloocta [1,2,3-de]naphthalen-2-yl)-5-methyl- 4-(trifluoromethyl)aniline | 6.69 (s, 1H), 6.46- 6.40 (m, 1H), 4.48- 4.20 (m, 5H), 4.16- 3.68 (m, 4H), 3.61- 3.53 (m, 2H), 2.96- 2.88 (m, 2H), 2.49- 2.34 (m, 5H), 2.22- 1.83 (m, 5H), 1.72- 1.62 (m, 2H), 1.10- 1.07 (m, 6H), 0.75- 0.69 (m, 2H), 0.55- 0.43 (m, 2H). | 672.5 |
| 65 | 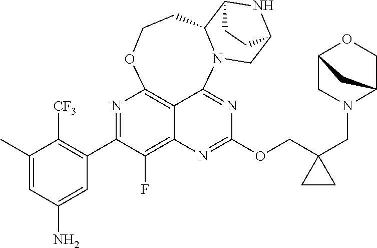 [SMILES: Cc1cc(N)cc(-c2nc3c4c(nc(OCC5(CN6C[C@H]7C[C@@H]6CO7)CC5)nc4c2F)N2C[C@H]4CC[C@H](N4)[C@H]2CCO3)c1C(F)(F)F] | 3-((6aR,7S,10R)-13-((1-(((1R,4R)- 2-oxa-5-azabicyclo[2.2.1]heptan-5- yl)methyl)cyclopropyl)methoxy)-1- fluoro-5,6,6a,7,8,9,10,11- octahydro-4-oxa-3,11a,12,14,15- pentaaza-7,10- methanocyclohepta[4,5]cycloocta [1,2,3-de]naphthalen-2-yl)-5-methyl- 4-(trifluoromethyl)aniline | 6.69 (s, 1H), 6.47- 6.40 (m, 1H), 4.51- 3.74 (m, 13H), 3.33- 3.30 (m, 2H), 3.12- 2.89 (m, 2H), 2.43 (s, 3H), 2.19-1.84 (m, 8H), 0.89-0.59 (m, 4H) | 656.5 |
| 66 | 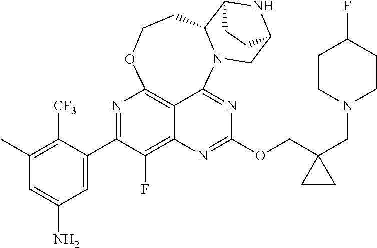 [SMILES: Cc1cc(N)cc(-c2nc3c4c(nc(OCC5(CN6CCC(F)CC6)CC5)nc4c2F)N2C[C@H]4CC[C@H](N4)[C@H]2CCO3)c1C(F)(F)F] | 3-((6aR,7S,10R)-1-fluoro-13-((1- ((4-fluoropiperidin-1- yl)methyl)cyclopropyl)methoxy)- 5,6,6a,7,8,9,10,11-octahydro-4-oxa- 3,11a,12,14,15-pentaaza-7,10- methanocyclohepta[4,5]cycloocta [1,2,3-de]naphthalen-2-yl)-5-methyl- 4-(trifluoromethyl)aniline | 6.69 (s, 1H), 6.47- 6.40 (m, 1H), 4.47- 3.92 (m, 10H), 3.26- 2.87 (m, 6H), 2.43 (s, 3H), 2.27-1.87 (m, 10H), 2.27-1.87 (m, 10H), 0.84-0.68 (m, 4H | 660.5 |
| 67 | 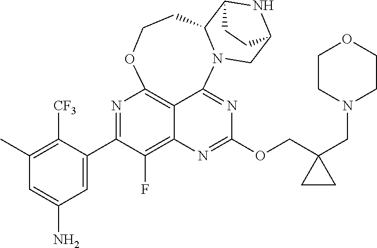 [SMILES: Cc1cc(N)cc(-c2nc3c4c(nc(OCC5(CN6CCOCC6)CC5)nc4c2F)N2C[C@H]4CC[C@H](N4)[C@H]2CCO3)c1C(F)(F)F] | 3-((6aR,7S,10R)-1-fluoro-13-((1- (morpholinomethyl)cyclopropyl) methoxy)-5,6,6a,7,8,9,10,11- octahydro-4-oxa-3,11a,12,14,15- pentaaza-7,10- methanocyclohepta[4,5]cycloocta [1,2,3-de]naphthalen-2-yl)-5-methyl- 4-(trifluoromethyl)aniline | 6.69 (s, 1H), 6.49- 6.38 (m, 1H), 4.46- 3.77 (m, 9H), 3.64- 3.63 (m, 4H), 2.52- 2.42 (m, 9H), 2.27- 1.86 (m, 6H), 0.72- 0.51 (m, 4H) | 644.5 |
| 68 | 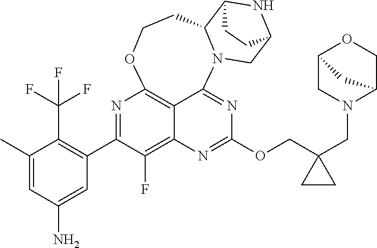 [SMILES: Cc1cc(N)cc(-c2nc3c4c(nc(OCC5(CN6C[C@@H]7C[C@H]6CO7)CC5)nc4c2F)N2C[C@H]4CC[C@H](N4)[C@H]2CCO3)c1C(F)(F)F] | 3-((6aR,7S,10R)-13-((1-(((1S,4S)- 2-oxa-5-azabicyclo[2.2.1]heptan-5- yl)methyl)cyclopropyl)methoxy)-1- fluoro-5,6,6a,7,8,9,10,11- octahydro-4-oxa-3,11a,12,14,15- pentaaza-7,10- methanocyclohepta[4,5]cycloocta [1,2,3-de]naphthalen-2-yl)-5-methyl- 4-(trifluoromethyl)aniline | 6.69 (s, 1H), 6.47- 6.43 (m, 1H), 4.43- 3.69 (m, 13H), 3.06- 2.77 (m, 4H), 2.42 (s, 3H), 2.11-1.84 (m, 8H), 0.76-0.61 (m, 4H) | 656.5 |
| 69 | 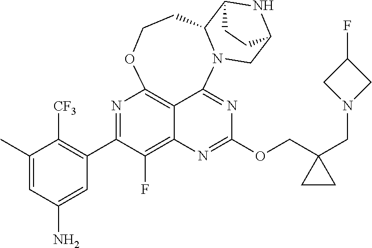 [SMILES: Cc1cc(N)cc(-c2nc3c4c(nc(OCC5(CN6CC(F)C6)CC5)nc4c2F)N2C[C@H]4CC[C@H](N4)[C@H]2CCO3)c1C(F)(F)F] | 3-((6aR,7S,10R)-1-fluoro-13-((1- ((3-fluoroazetidin-1- yl)methyl)cyclopropyl)methoxy)- 5,6,6a,7,8,9,10,11-octahydro-4-oxa- 3,11a,12,14,15-pentaaza-7,10- methanocyclohepta[4,5]cycloocta [1,2,3-de]naphthalen-2-yl)-5-methyl- 4-(trifluoromethyl)aniline | 6.71 (s, 1H), 6.47- 6.43 (m, 1H), 5.38- 5.27 (m, 1H), 4.53- 4.08 (m, 13H), 3.31- 3.09 (m, 3H), 2.65- 2.03 (m, 8H), 0.85- 0.78 (m, 4H) | 632.5 |
| 70 | 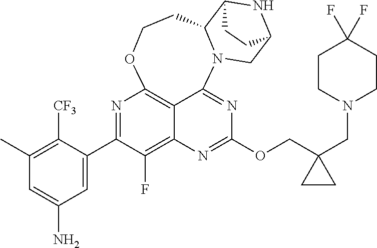 [SMILES: Cc1cc(N)cc(-c2nc3c4c(nc(OCC5(CN6CCC(F)(F)CC6)CC5)nc4c2F)N2C[C@H]4CC[C@H](N4)[C@H]2CCO3)c1C(F)(F)F] | 3-((6aR,7S,10R)-13-((1-((4,4- difluoropiperidin-1- yl)methyl)cyclopropyl)methoxy)-1- fluoro-5,6,6a,7,8,9,10,11- octahydro-4-oxa-3,11a,12,14,15- pentaaza-7,10- methanocyclohepta[4,5]cycloocta [1,2,3-de]naphthalen-2-yl)-5-methyl- 4-(trifluoromethyl)aniline | 6.69 (s, 1H), 6.45 (m, 1H), 4.44-3.91 (m, 9H), 2.65-1.94 (m, 19H), 0.73-0.52 (m, 4H) | 678.5 |
| 71 | 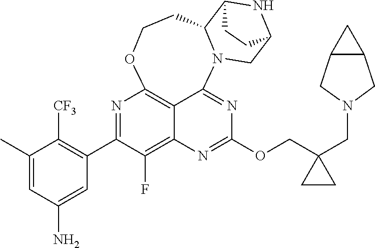 [SMILES: Cc1cc(N)cc(-c2nc3c4c(nc(OCC5(CN6CC7CC7C6)CC5)nc4c2F)N2C[C@H]4CC[C@H](N4)[C@H]2CCO3)c1C(F)(F)F] | 3-((6aR,7S,10R)-13-((1-((3- azabicyclo[3.1.0]hexan-3- yl)methyl)cyclopropyl)methoxy)-1- fluoro-5,6,6a,7,8,9,10,11- octahydro-4-oxa-3,11a,12,14,15- pentaaza-7,10- methanocyclohepta[4,5]cycloocta [1,2,3-de]naphthalen-2-yl)-5-methyl- 4-(trifluoromethyl)aniline | 6.68 (s, 1H), 6.47- 6.41(m, 1H), 4.38- 3.44 (m, 10H), 3.17- 3.07 (m, 1H), 2.66- 1.73 (m, 12H), 1.44- 1.29 (m, 2H), 0.66- 0.31 (m, 6H) | 640.5 |

Examples 72-73: The following examples were prepared in a manner similar to that in Example 36 by replacing the corresponding boronic acid in the Suzuki coupling reactions.

| | | | | MS |
|---|---|---|---|---|
| | | | | (ESI, |
| | | | | m/e) |
| | | | 1H NMR (500 MHz, | [M + |
| Example | Structure | Compound Name | CD₃OD) δ | H]⁺ |
| 72 | 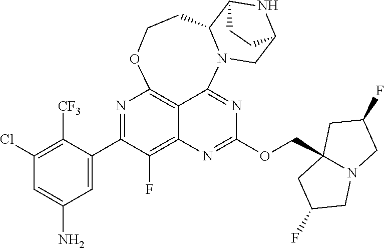 | 3-chloro-5-((6aR,7S,10R)-13- (((2R,6R)-2,6-difluorotetrahydro- 1H-pyrrolizin-7a(5H)-yl)methoxy)- 1-fluoro-5,6,6a,7,8,9,10,11- octahydro-4-oxa-3,11a,12,14,15- pentaaza-7,10- methanocyclohepta[4,5]cycloocta [1,2,3-de]naphthalen-2-yl)-4- (trifluoromethyl)aniline | 6.88 (s, 1H), 6.55- 6.49 (m, 1H), 5.39- 5.25 (m, 2H), 4.57- 4.26 (m, 5H), 4.09- 3.95 (m, 2H), 3.66 (s, 1H), 3.58-3.44 (m, 2H), 3.29-3.21 (m, 2H), 3.19-3.15 (m, 1H), 2.50-2.42 (m, 1H), 2.40-2.20 (m, 3H), 2.13-2.08 (m, 2H), 1.97-1.79 (m, 4H). | 670.4 |
| 73 | 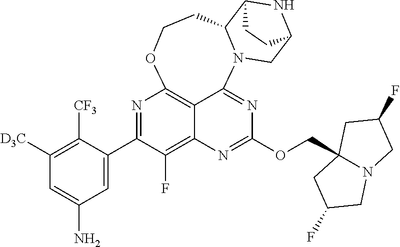 | 3-((6aR,7S,10R)-13-(((2R,6R)-2,6- difluorotetrahydro-1H-pyrrolizin- 7a(5H)-yl)methoxy)-1-fluoro- 5,6,6a,7,8,9,10,11-octahydro-4-oxa- 3,11a,12,14,15-pentaaza-7,10- methanocyclohepta[4,5]cycloocta [1,2,3-de]naphthalen-2-yl)-5-(methyl- d3)-4-(trifluoromethyl)aniline | 6.68 (s, 1H), 6.46- 6.42 (m, 1H), 5.38- 5.25 (m, 2H), 4.42- 4.25 (m, 5H), 4.08- 3.92 (m, 2H), 3.65 (s, 1H), 3.58-3.48 (m, 2H), 3.29-3.13 (m, 3H), 2.53-2.40 (m, 1H), 2.40-2.18 (m, 3H), 2.15-2.06 (m, 2H), 1.99-1.72 (m, 4H). | 653.5 |

Examples 74: The following examples were prepared in a manner similar to that in Example 37 by replacing (5)-1-((S)-1-methylpyrrolidin-2-yl)ethan-1-ol with (S)-1-((2S,4R)-4-fluoro-1-methylpyrrolidin-2-yl)ethan-1-ol.

| | | | | MS |
|---|---|---|---|---|
| | | | | (ESI, |
| | | | | m/e) |
| | | | 1H NMR (500 MHz, | [M + |
| Example | Structure | Compound Name | CD₃OD) δ | H]⁺ |
| 74 | 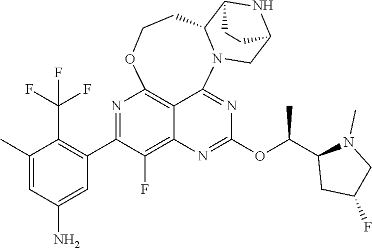 [SMILES: Cc1cc(N)cc(-c2nc3c4c(nc(O[C@@H](C)[C@@H]5C[C@@H](F)CN5C)nc4c2F)N2C[C@H]4CC[C@H](N4)[C@H]2CCO3)c1C(F)(F)F] | 3-((6aR,7S,10R)-1-fluoro-13-((S)- 1-((2S,4R)-4-fluoro-1- methylpyrrolidin-2-yl)ethoxy)- 5,6,6a,7,8,9,10,11-octahydro-4-oxa- 3,11a,12,14,15-pentaaza- 7,10methanocyclohepta[4,5]cycloocta [1,2,3-de]naphthalen-2-yl)-5- methyl-4-(trifluoromethyl)aniline | δ 6.70(s, 1H), 6.47- 6.41 (m, 1H), 5.38- 5.17 (m, 2H), 4.33- 3.36 (m, 9H), 2.99- 1.91 (m, 1H), 2.74 (s, 3H), 2.42-1.94 (m, 11H), 1.44-1.43 (m, 2H). | 620.6 |

Examples 75-76: The following examples were prepared in a manner similar to that in Example 37 by replacing (5)-1-((5)-1-methylpyrrolidin-2-yl)ethan-1-ol with the corresponding alcohol as a mixture of two isomers, followed by further separation via chiral Prep-HPLC (Column: i-Cellulose-5, 21.2 mm×250 mm, 5 μm; Mobile Phase A: n-Hexane, Mobile Phase B: EtOH (with 0.2% 2M NH₃ in MeOH); Flow Rate: 18 mL/min; Temperature: 25° C.; Gradient: 50% B in 18 min).

| | | | | MS |
|---|---|---|---|---|
| | | | | (ESI, |
| | | | | m/e) |
| | | | 1H NMR (500 MHz, | [M + |
| Example | Structure | Compound Name | CD₃OD) δ | H]⁺ |
| 75 | 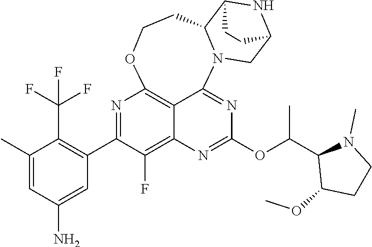 [SMILES: CO[C@H]1CCN(C)[C@@H]1C(C)Oc1nc2c3c(nc(-c4cc(N)cc(C)c4C(F)(F)F)c(F)c3n1)OCC[C@@H]1[C@@H]3CC[C@H](CN21)N3] | 3-((6aR,7S,10R)-1-fluoro-13-(1- ((2R,3S)-3-methoxy-1- methylpyrrolidin-2-yl)ethoxy)- 5,6,6a,7,8,9,10,11-octahydro-4-oxa- 3,11a,12,14,15-pentaaza-7,10- methanocyclohepta[4,5]cycloocta [1,2,3-de]naphthalen-2-yl)-5-methyl- 4-(trifluoromethyl)aniline | 6.69 (s, 1H), 6.47- 6.41 (m, 1H), 5.35- 5.33 (m, 1H), 4.57- 3.90 (m, 8H), 3.30- 3.09 (m,3H), 2.92- 2.56 (m, 6H), 2.42 (s, 3H), 2.13-1.90 (m, 8H), 1.48-1.47 (m, 3H). | 632.5 |
| | (isomer 1) | | | |
| 76 | 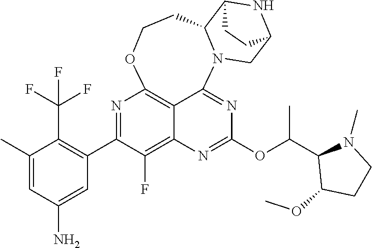 [SMILES: CO[C@H]1CCN(C)[C@@H]1C(C)Oc1nc2c3c(nc(-c4cc(N)cc(C)c4C(F)(F)F)c(F)c3n1)OCC[C@@H]1[C@@H]3CC[C@H](CN21)N3] | 3-((6aR,7S,10R)-1-fluoro-13-(1- ((2R,3S)-3-methoxy-1- methylpyrrolidin-2-yl)ethoxy)- 5,6,6a,7,8,9,10,11-octahydro-4-oxa- 3,11a,12,14,15-pentaaza-7,10- methanocyclohepta[4,5]cycloocta [1,2,3-de]naphthalen-2-yl)-5-methyl- 4-(trifluoromethyl)aniline | 6.69 (s, 1H), 6.47- 6.41 (m, 1H), 5.69- 5.61 (m, 1H), 4.32- 3.87 (m, 8H), 3.37(s, 1H), 3.16-3.11 (m, 2H), 2.73-2.58 (m, 5H), 2.42 (s, 3H), 2.25-1.97 (m, 8H), 1.48-1.47 (m, 3H). | 632.5 |
| | (isomer 2) | | | |

### Example 77: 3-((6aR,7S,10R)-1-fluoro-13-((1-(pyrrolidin-1-ylmethyl)cyclopropyl)methoxy)-5,6,6a,7,8,9,10,11-octahydro-4-oxa-3,11a,12,14,15-pentaaza-7,10-methanocyclohepta[4,5]cycloocta[1,2,3-de]naphthalen-2-yl)-5-methyl-4-(trifluoromethyl)aniline

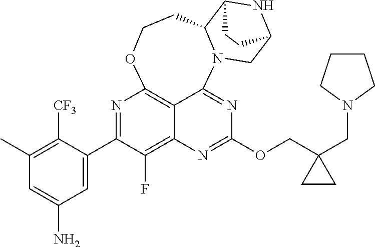 [SMILES: Cc1cc(N)cc(-c2nc3c4c(nc(OCC5(CN6CCCC6)CC5)nc4c2F)N2C[C@H]4CC[C@H](N4)[C@H]2CCO3)c1C(F)(F)F]

### Synthetic Route

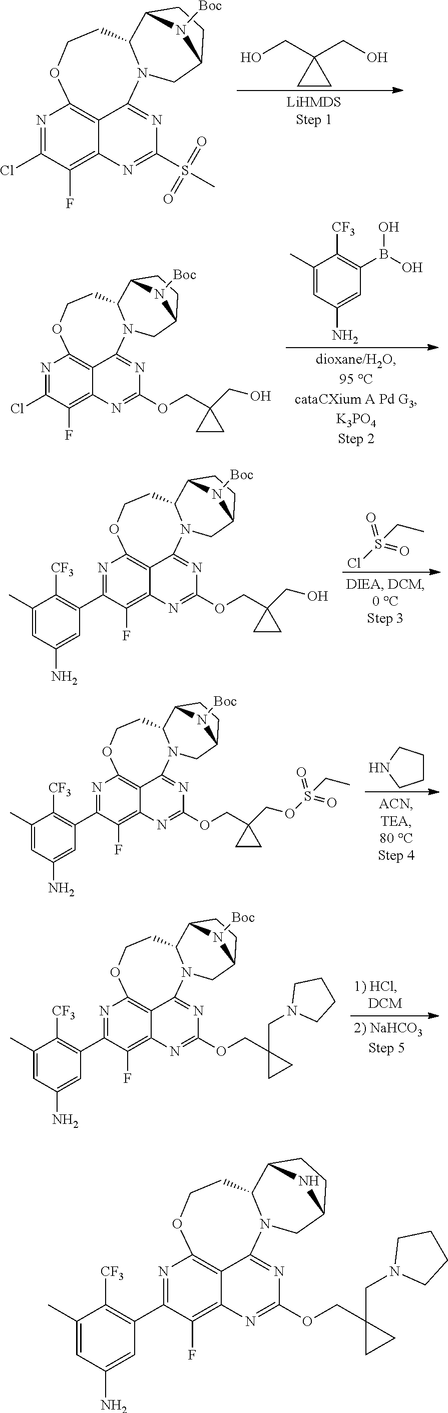

### Step 1: tert-butyl (6aR,7S,10R)-2-chloro-1-fluoro-13-((1-(hydroxymethyl)cyclopropyl)methoxy)-5,6,6a,7,8,9,10,11-octahydro-4-oxa-3,11a,12,14,15-pentaaza-7,10-methanocyclohepta[4,5]cycloocta [1,2,3-de]naphthalene-15-carboxylate

To a solution of cyclopropane-1,1-diyldimethanol (567 mg, 5.56 mmol) in THF (30 mL) was added LHMDS (1N, 5.5 mL, 5.5 mmol). The resulting mixture was stirred for 10 minutes at 0° C., followed by addition of tert-butyl (6aR,7S,10R)-2-chloro-1-fluoro-13-(methylsulfonyl)-5,6,6a,7,8,9,10,11-octahydro-4-oxa-3,11a,12,14,15-pentaaza-7,10-methanocyclohepta[4,5]cycloocta[1,2,3-de]naphthalene-15-carboxylate (1.9 g, 3.7 mmol) in THF (20 mL) at 0° C. The resulting mixture was stirred for 1 hour at room temperature. After the reaction was complete as indicated by TLC, the reaction mixture was concentrated to give the crude residue. The residue was purified by column chromatography, eluted with 0 to 10% MeOH in DCM to give the title product (1.9 g, 96%). MS (ESI, m/e) [M+1]⁺ 536.4.

### Step 2: tert-butyl (6aR,7S,10R)-2-(5-amino-3-methyl-2-(trifluoromethyl)phenyl)-1-fluoro-13-((1-(hydroxymethyl)cyclopropyl)methoxy)-5,6,6a,7,8,9,10,11-octahydro-4-oxa-3,11a,12,14,15-pentaaza-7,10-methanocyclohepta[4,5]cycloocta[1,2,3-de]naphthalene-15-carboxylate

To the mixture of tert-butyl (6aR,7S,10R)-2-chloro-1-fluoro-13-((1-(hydroxymethyl)cyclopropyl) methoxy)-5,6,6a,7,8,9,10,11-octahydro-4-oxa-3,11a,12,14,15-pentaaza-7,10-methanocyclohepta [4,5]cycloocta[1,2,3-de]naphthalene-15-carboxylate (1.9 g, 3.55 mmol), (5-amino-3-methyl-2-(trifluoromethyl)phenyl)boronic acid (1.56 g, 7.12 mmol), cataCXium A Pd G3 (260 mg, 0.36 mmol), and K₃PO₄ (2.26 g, 10.7 mmol) was added 1,4-dioxane (50 mL) and water (10 mL) at room temperature. The resulting mixture was degassed by bubbling nitrogen through for 10 minutes and then stirred at 95° C. for 3 hours under a N₂ atmosphere. After the reaction was complete as indicated by TLC, the reaction mixture was concentrated, dissolved in DCM (20 mL), and filtered through Celite. The filtrate was concentrated to give the crude residue. The residue was purified by column chromatography, eluted with 0 to 10% MeOH in DCM to give the title product (1.83 g, 76%). MS (ESI, m/e) [M+1]⁺ 675.4.

### Step 3: tert-butyl (6aR,7S,10R)-2-(5-amino-3-methyl-2-(trifluoromethyl)phenyl)-13-((1-(((ethylsulfonyl)oxy)methyl)cyclopropyl)methoxy)-1-fluoro-5,6,6a,7,8,9,10,11-octahydro-4-oxa-3,11a,12,14,15-pentaaza-7,10-methanocyclohepta[4,5]cycloocta[1,2,3-de]naphthalene-15-carboxylate

To a solution of tert-butyl (6aR,7S,10R)-2-(5-amino-3-methyl-2-(trifluoromethyl)phenyl)-1-fluoro-13-((1-(hydroxymethyl)cyclopropyl)methoxy)-5,6,6a,7,8,9,10,11-octahydro-4-oxa-3,11a,12,14,15-pentaaza-7,10-methanocyclohepta[4,5]cycloocta[1,2,3-de]naphthalene-15-carboxylate (1 g, 1.48 mmol) and DIPEA (574 mg, 4.45 mmol) in DCM (30 mL) was added a solution of ethanesulfonyl chloride (228 mg, 1.78 mmol) in DCM (5 mL) at 0° C. The reaction mixture was stirred at 0° C. for 0.5 hours, and then washed with sat. NaHCO₃ aqueous solution. The organic phase was dried by Na₂SO₄ and filtered through Celite. The filtrate was concentrated to give the crude product (1.13 g, 100%). MS (ESI, m/e) [M+1]⁺ 767.5.

### Step 4: tert-butyl (6aR,7S,10R)-2-(5-amino-3-methyl-2-(trifluoromethyl)phenyl)-1-fluoro-13-((1-(pyrrolidin-1-ylmethyl)cyclopropyl)methoxy)-5,6,6a,7,8,9,10,11-octahydro-4-oxa-3,11a,12,14,15-pentaaza-7,10-methanocyclohepta[4,5]cycloocta[1,2,3-de]naphthalene-15-carboxylate

To a solution of tert-butyl (6aR,7S,10R)-2-(5-amino-3-methyl-2-(trifluoromethyl)phenyl)-13-((1-(((ethylsulfonyl)oxy)methyl)cyclopropyl)methoxy)-1-fluoro-5,6,6a,7,8,9,10,11-octahydro-4-oxa-3,11a,12,14,15-pentaaza-7,10-methanocyclohepta[4,5]cycloocta[1,2,3-de]naphthalene-15-carboxylate (37.6 mg, 0.05 mmol) as crude and pyrrolidine (35.5 mg, 0.5 mmol) in CH₃CN (2 mL) was added TEA (10.1 mg, 0.1 mmol), stirred at 80° C. for 4 hours, the cooled resulting solution was concentrated and washed by NaHCO₃ aqueous solution, dried by Na₂SO₄ and concentrated to give the title product (30 mg). MS (ESI, m/e) [M+1]⁺ 728.5.

### Steps 5: 3-((6aR,7S,10R)-1-fluoro-13-((1-(pyrrolidin-1-ylmethyl)cyclopropyl)methoxy)-5,6,6a,7,8,9,10,11-octahydro-4-oxa-3,11a,12,14,15-pentaaza-7,10-methanocyclohepta[4,5]cycloocta[1,2,3-de]naphthalen-2-yl)-5-methyl-4-(trifluoromethyl)aniline

To a solution of tert-butyl (6aR,7S,10R)-2-(5-amino-3-methyl-2-(trifluoromethyl)phenyl)-1-fluoro-13-((1-(pyrrolidin-1-ylmethyl)cyclopropyl)methoxy)-5,6,6a,7,8,9,10,11-octahydro-4-oxa-3,11a,12,14,15-pentaaza-7,10-methanocyclohepta[4,5]cycloocta[1,2,3-de]naphthalene-15-carboxylate (30 mg, crude) in DCM (2 mL) was added 4 N HCl in dioxane (2 mL) at room temperature. The resulting mixture was stirred at room temperature for 2 hours. The resulting solution was concentrated, diluted with NaHCO₃ aqueous solution, and extracted with DCM (10 mL) 2 times. The combined DCM phase was dried by Na₂SO₄ and concentrated to give the crude residue which was further purified by Prep-HPLC (Column: Waters X-Select C18, 19*150 mm, 5 μm; Mobile Phase A: H2O (0.1% FA), Mobile Phase B: MeCN (0.1% FA); Flow Rate: 17 mL/min; Temperature: 25° C.; Detector: UV 254 nm and 214 nm; Gradient: 12 to 35% B within 1 min to 11 min) to give the title product (1.26 mg). ¹H NMR (500 MHz, CD₃OD) δ 6.69 (s, 1H), 6.46-6.39 (m, 1H), 4.41-4.10 (m, 7H), 3.45-3.37 (m, 4H), 3.16-2.93 (m, 4H), 2.42 (s, 3H), 2.15-1.62 (m, 10H), 0.90-0.73 (m, 4H). MS (ESI, m/e) [M+1]⁺ 628.5.

Examples 78-109: The following examples were prepared in a manner similar to that in Example 77 by replacing pyrrolidine with the corresponding alkyl amines.

| | | | | MS |
|---|---|---|---|---|
| | | | | (ESI, |
| | | | 1H NMR | m/e) |
| Ex- | | | (500 MHz, | [M + |
| ample | Structure | Compound Name | CD₃OD) δ | H]⁺ |
| 78 | 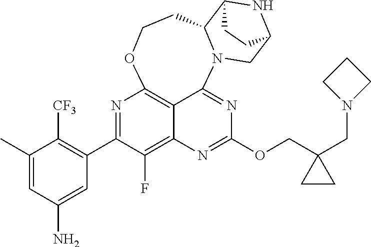 [SMILES: Cc1cc(N)cc(-c2nc3c4c(nc(OCC5(CN6CCC6)CC5)nc4c2F)N2C[C@H]4CC[C@H](N4)[C@H]2CCO3)c1C(F)(F)F] | 3-((6aR,7S,10R)- 13-((1-(azetidin-1- ylmethyl)cyclopropyl) methoxy)-1- fluoro-5,6,6a,7,8,9,10,11- octahydro-4-oxa-3, 11a,12,14,15- pentaaza-7,10- methanocyclohepta [4,5]cycloocta [1,2,3-de]naphthalen- 2-yl)-5-methyl- 4-(trifluoromethyl)aniline | 6.70 (s, 1H), 6.47- 6.43 (m, 1H), 4.33- 3.95 (m, 13H), 3.37- 3.31 (m, 3H), 2.59- 2.29 (m, 5H), 2.22- 1.89 (m, 5H), 0.92- 0.74 (m, 4H) | 614.5 |

| | | | | |
|---|---|---|---|---|
| 79 | 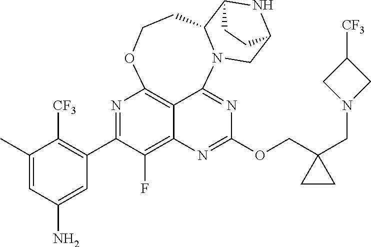 [SMILES: Cc1cc(N)cc(-c2nc3c4c(nc(OCC5(CN6CC(C(F)(F)F)C6)CC5)nc4c2F)N2C[C@H]4CC[C@H](N4)[C@H]2CCO3)c1C(F)(F)F] | 3-((6aR,7S,10R)- 1-fluoro-13-((1- ((3-(trifluoromethyl) azetidin-1-yl)methyl) cyclopropyl)methoxy)- 5,6,6a,7,8,9,10,11- octahydro-4-oxa- 3,11a,12,14,15-pentaaza-7,10- methanocyclohepta [4,5]cycloocta [1,2,3-de]naphthalen- 2-yl)-5-methyl- 4-(trifluoromethyl)aniline | 6.69(s, 1H), 6.47- 6.41 (m, 1H), 4.34- 3.58 (m, 11H), 3.34- 3.31(m, 5H), 2.63- 2.57 (m, 2H), 2.42- 1.88 (m, 9H), 0.64- 0.55 (m, 4H) | 682.5 |

| | | | | |
|---|---|---|---|---|
| 80 | 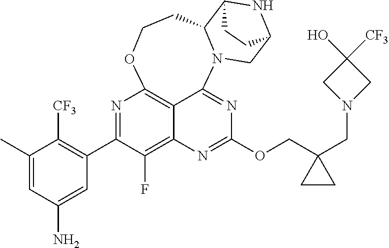 [SMILES: Cc1cc(N)cc(-c2nc3c4c(nc(OCC5(CN6CC(O)(C(F)(F)F)C6)CC5)nc4c2F)N2C[C@H]4CC[C@H](N4)[C@H]2CCO3)c1C(F)(F)F] | 1-((1-((((6aR,7S,10R)- 2-(5-amino- 3-methyl-2- (trifluoromethyl)phenyl)- 1-fluoro- 5,6,6a,7,8,9,10,11- octahydro-4-oxa- 3,11a,12,14,15-pentaaza-7,10- methanocyclohepta[4,5] cycloocta [1,2,3-de]naphthalen-13- yl)oxy)methyl)cyclopropyl) methyl)- 3-(trifluoromethyl) azetidin-3-ol | 6.70 (s, 1H), 6.47- 6.43 (m, 1H), 4.42- 3.63 (m, 12H), 3.33- 3.31 (m, 3H), 2.42 (s. 3H), 2.10-1.98 (m, 10H), 0.85-0.75 (m, 4H) | 698.5 |

| | | | | |
|---|---|---|---|---|
| 81 | 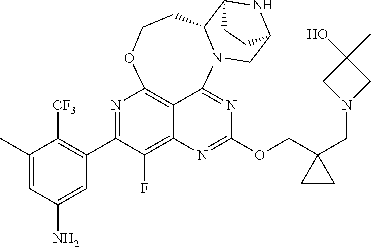 | 1-((1-((((6aR,7S, 10R)-2-(5-amino- 3-methyl-2- (trifluoromethyl) phenyl)-1-fluoro- 5,6,6a,7,8,9,10,11- octahydro-4-oxa- 3,11a,12,14,15-pentaaza- 7,10- methanocyclohepta [4,5]cycloocta [1,2,3-de]naphthalen-13- yl)oxy)methyl) cyclopropyl)methyl)- 3-methylazetidin-3-ol | 6.70 (s, 1H), 6.47- 6.41 (m, 1H), 4.36- 3.60(m, 13H), 3.35- 3.31 (m, 3H), 2.42 (s. 3H), 2.18-1.83 (m, 5H), 1.51 (s. 3H), 0.85-0.75 (m, 4H) | 644.5 |

| | | | | |
|---|---|---|---|---|
| 82 | 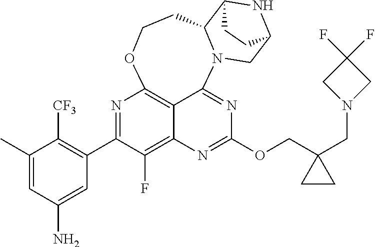 [SMILES: Cc1cc(N)cc(-c2nc3c4c(nc(OCC5(CN6CC(F)(F)C6)CC5)nc4c2F)N2C[C@H]4CC[C@H](N4)[C@H]2CCO3)c1C(F)(F)F] | 3-((6aR,7S,10R)-13-((1-((3,3- difluoroazetidin-1- yl)methyl)cyclopropyl) methoxy)-1- fluoro-5,6,6a,7,8,9,10,11- octahydro-4- oxa-3,11a,12,14,15- pentaaza-7,10- methanocyclohepta [4,5]cycloocta [1,2,3-de]naphthalen- 2-yl)-5-methyl- 4-(trifluoromethyl)aniline | 6.70 (s, 1H), 6.47- 6.41 (m, 1H), 4.58- 3.44 (m, 14H), 2.68- 2.65 (m, 2H), 2.42 (s. 3H), 2.15-2.03 (m, 8H), 0.67-0.55 (m, 4H) | 650.5 |

| | | | | |
|---|---|---|---|---|
| 83 | 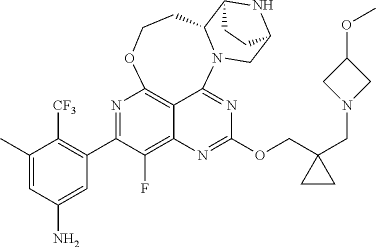 | 3-((6aR,7S,10R)- 1-fluoro-13-((1- ((3-methoxyazetidin-1- yl)methyl)cyclopropyl) methoxy)- 5,6,6a,7,8,9,10,11- octahydro-4-oxa- 3,11a,12,14,15- pentaaza-7,10- methanocyclohepta [4,5]cycloocta [1,2,3-de]naphthalen- 2-yl)-5-methyl- 4-(trifluoromethyl)aniline | 6.69 (s, 1H), 6.47- 6.43 (m, 1H), 4.56- 3.89 (m, 14H), 3.22- 3.16 (m, 5H), 2.42 (s. 3H), 2.22-2.04 (m, 6H), 0.82-0.81 (m, 4H) | 644.5 |

| | | | | |
|---|---|---|---|---|
| 84 | 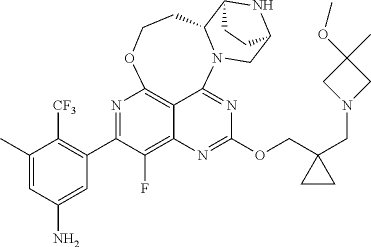 | 3-((6aR,7S,10R)- 1-fluoro-13-((1- ((3-methoxy-3- methylazetidin-1- yl)methyl)cyclopropyl) methoxy)- 5,6,6a,7,8,9,10,11- octahydro-4-oxa- 3,11a,12,14,15- pentaaza-7,10- methanocyclohepta [4,5]cycloocta [1,2,3-de]naphthalen- 2-yl)-5-methyl- 4-(trifluoromethyl)aniline | 6.70 (s, 1H), 6.47- 6.43 (m, 1H), 4.36- 3.80 (m, 13H), 3.28- 3.16 (m, 5H), 2.42 (s. 3H), 2.14-1.92 (m, 6H), 1.49 (s. 3H), 0.84-0.80 (m, 4H) | 658.5 |

| | | | | |
|---|---|---|---|---|
| 85 | 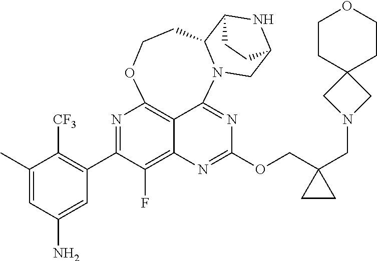 [SMILES: Cc1cc(N)cc(-c2nc3c4c(nc(OCC5(CN6CC7(CCOCC7)C6)CC5)nc4c2F)N2C[C@H]4CC[C@H](N4)[C@H]2CCO3)c1C(F)(F)F] | 3-((6aR,7S,10R)- 13-((1-((7-oxa-2- azaspiro[3.5]nonan-2- yl)methyl)cyclopropyl) methoxy)-1- fluoro-5,6,6a, 7,8,9,10,11- octahydro-4-oxa- 3,11a,12,14,15- pentaaza[7,10] methanocyclohepta [4,5]cycloocta [1,2,3-de]naphthalen-2- yl)-5-methyl-4- (trifluoromethyl)aniline | 6.70 (s, 1H), 6.45- 6.41 (m, 1H), 4.37- 3.45 (m, 19H), 2.42 (s, 3H), 2.12-1.75 (m, 10H), 0.87-0.84 (m, 4H) | 684.5 |

| | | | | |
|---|---|---|---|---|
| 86 | 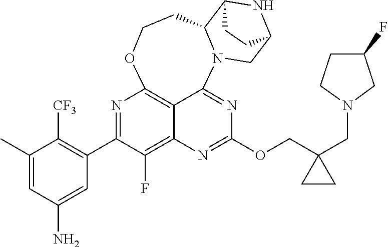 [SMILES: Cc1cc(N)cc(-c2nc3c4c(nc(OCC5(CN6CC[C@@H](F)C6)CC5)nc4c2F)N2C[C@H]4CC[C@H](N4)[C@H]2CCO3)c1C(F)(F)F] | 3-((6aR,7S,10R)- 1-fluoro-13-((1- (((R)-3-fluoropyrrolidin- 1-yl)methyl)cyclopropyl) methoxy)- 5,6,6a,7,8,9,10,11- octahydro-4-oxa- 3,11a,12,14,15- pentaaza-7,10- methanocyclohepta [4,5]cycloocta [1,2,3-de]naphthalen- 2-yl)-5-methyl- 4-(trifluoromethyl) aniline | 6.69 (s, 1H), 6.44- 6.40 (m,1H), 5.21- 5.10 (m, 1H),4.52- 3.78 (m, 9H), 3.16- 2.58 (m, 6H), 2.42 (s, 3H), 2.21-1.93 (m, 8H), 0.74-0.58 (m, 4H). | 646.5 |

| | | | | |
|---|---|---|---|---|
| 87 | 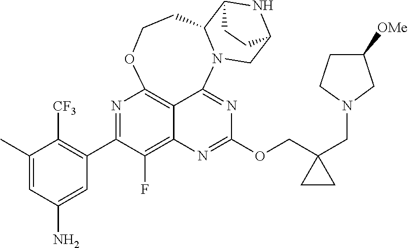 [SMILES: CO[C@@H]1CCN(CC2(COc3nc4c5c(nc(-c6cc(N)cc(C)c6C(F)(F)F)c(F)c5n3)OCC[C@@H]3[C@@H]5CC[C@H](CN43)N5)CC2)C1] | 3-((6aR,7S,10R)- 1-fluoro-13-((1- (((R)-3- methoxypyrrolidin-1- yl)methyl)cyclopropyl) methoxy)- 5,6,6a,7,8,9,10,11- octahydro-4-oxa- 3,11a,12,14,15- pentaaza-7,10- methanocyclohepta [4,5]cycloocta [1,2,3-de]naphthalen- 2-yl)-5-methyl- 4-(trifluoromethyl)aniline | 6.68(s, 1H), 6.46- 6.42 (m, 1H), 4.44- 3.44 (m, 10H), 3.25(s, 3H), 2.89- 2.68 (m, 6H), 2.42 (s, 3H), 2.21-1.75 (m, 8H), 0.75-0.60 (m, 4H). | 658.5 |

| | | | | |
|---|---|---|---|---|
| 88 | 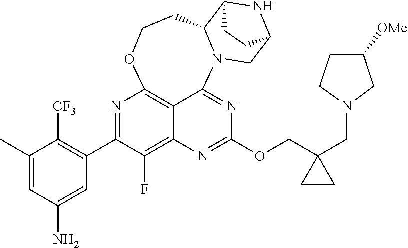 [SMILES: CO[C@H]1CCN(CC2(COc3nc4c5c(nc(-c6cc(N)cc(C)c6C(F)(F)F)c(F)c5n3)OCC[C@@H]3[C@@H]5CC[C@H](CN43)N5)CC2)C1] | 3-((6aR,7S,10R)- 1-fluoro-13-((1-(((S)-3- methoxypyrrolidin-1- yl)methyl)cyclopropyl) methoxy)- 5,6,6a,7,8,9,10,11- octahydro-4-oxa- 3,11a,12,14,15- pentaaza-7,10- methanocyclohepta [4,5]cycloocta [1,2,3-de]naphthalen- 2-yl)-5-methyl- 4-(trifluoromethyl) aniline | 6.68(s, 1H), 6.46- 6.41 (m,1H), 4.45- 3.48 (m, 10H), 3.26(s, 3H), 2.89- 2.68 (m, 6H), 2.42 (s, 3H), 2.11-1.81 (m, 8H), 0.74-0.60 (m, 4H). | 658.5 |

| | | | | |
|---|---|---|---|---|
| 89 | 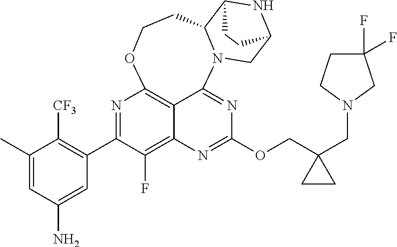 [SMILES: Cc1cc(N)cc(-c2nc3c4c(nc(OCC5(CN6CCC(F)(F)C6)CC5)nc4c2F)N2C[C@H]4CC[C@H](N4)[C@H]2CCO3)c1C(F)(F)F] | 3-((6aR,7S,10R)- 13-((1-((3,3- difluoropyrrolidin-1- yl)methyl)cyclopropyl) methoxy)-1- fluoro-5,6,6a,7,8,9,10,11- octahydro-4-oxa- 3,11a,12,14,15- pentaaza-7,10- methanocyclohepta [4,5]cycloocta [1,2,3-de]naphthalen- 2-yl)-5-methyl- 4-(trifluoromethyl) aniline | 6.69 (s, 1H), 6.44- 6.40 (m,1H), 4.47- 3.44 (m, 9H), 2.89- 2.60 (m, 6H), 2.42 (s, 3H), 2.12-1.93 (m, 8H), 0.69-0.52(m, 4H). | 664.5 |

| | | | | |
|---|---|---|---|---|
| 90 | 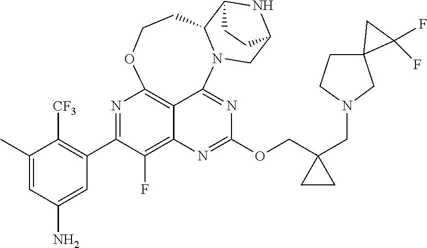 [SMILES: Cc1cc(N)cc(-c2nc3c4c(nc(OCC5(CN6CCC7(C6)CC7(F)F)CC5)nc4c2F)N2C[C@H]4CC[C@H](N4)[C@H]2CCO3)c1C(F)(F)F] | 3-((6aR,7S,10R)- 13-((1-((1,1- difluoro-5-azaspiro [2.4]heptan-5- yl)methyl)cyclopropyl) methoxy)-1- fluoro-5,6,6a,7,8,9,10,11- octahydro-4-oxa-3, 11a,12,14,15- pentaaza[7,10] methanocyclohepta[4,5] cycloocta[1,2,3-de] naphthalen-2-yl)-5-methyl- 4-(trifluoromethyl)aniline | 6.69(s, 1H), 6.45- 6.42 (m,1H), 4.46- 3.73(m, 9H), 2.91- 2.60 (m, 6H), 2.42 (s, 3H), 2.10-1.73 (m, 8H), 1.34-1.26 (m, 2H), 0.71-0.55(m, 4H). | 690.5 |

(mixture of two
isomers)

| | | | | |
|---|---|---|---|---|
| 91 | 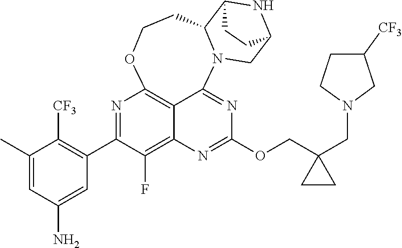 [SMILES: Cc1cc(N)cc(-c2nc3c4c(nc(OCC5(CN6CCC(C(F)(F)F)C6)CC5)nc4c2F)N2C[C@H]4CC[C@H](N4)[C@H]2CCO3)c1C(F)(F)F] | 3-((6aR,7S,10R)-1- fluoro-13-((1- ((3-(trifluoromethyl) pyrrolidin-1- yl)methyl)cyclopropyl) methoxy)- 5,6,6a,7,8,9,10,11- octahydro-4-oxa- 3,11a,12,14,15- pentaaza-7,10- methanocyclohepta [4,5]cycloocta [1,2,3-de]naphthalen- 2-yl)-5-methyl-4- (trifluoromethyl)aniline | 6.69 (s, 1H), 6.44- 6.43 (m,1H), 4.34- 3.63(m, 10H), 2.89- 2.60 (m, 6H), 2.42 (s, 3H), 2.22-1.93 (m, 8H), 0.71-0.53(m, 4H). | 696.5 |

(mixture of two isomers)

| | | | | |
|---|---|---|---|---|
| 92 | 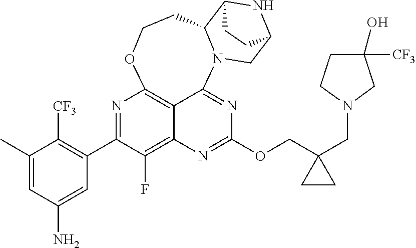 [SMILES: Cc1cc(N)cc(-c2nc3c4c(nc(OCC5(CN6CCC(O)(C(F)(F)F)C6)CC5)nc4c2F)N2C[C@H]4CC[C@H](N4)[C@H]2CCO3)c1C(F)(F)F] | 1-((1-((((6aR,7S, 10R)-2-(5-amino- 3-methyl-2- (trifluoromethyl) phenyl)-1-fluoro- 5,6,6a,7,8,9,10,11- octahydro-4-oxa- 3,11a,12,14,15- pentaaza-7,10- methanocyclohepta [4,5]cycloocta[1,2,3-de] naphthalen-13-yl)oxy) methyl)cyclopropyl) methyl)-3-(trifluoromethyl) pyrrolidin-3-ol | 6.69 (s, 1H), 6.44- 6.43 (m,1H), 4.44- 3.88 (m, 9H), 2.91- 2.55 (m, 6H), 2.42 (s, 3H), 2.18-1.83 (m, 8H), 0.71-0.53(m, 4H) | 712.5 |
| | (mixture of two isomers) | | | |

| | | | | |
|---|---|---|---|---|
| 93 | 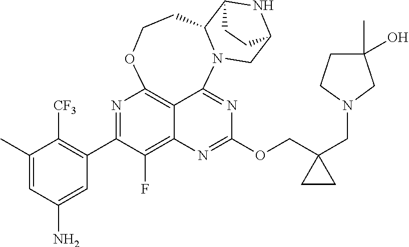 [SMILES: Cc1cc(N)cc(-c2nc3c4c(nc(OCC5(CN6CCC(C)(O)C6)CC5)nc4c2F)N2C[C@H]4CC[C@H](N4)[C@H]2CCO3)c1C(F)(F)F] | 1-((1-((((6aR,7S, 10R)-2-(5-amino- 3-methyl-2- (trifluoromethyl) phenyl)-1-fluoro- 5,6,6a,7,8,9,10,11- octahydro-4-oxa- 3,11a,12,14,15- pentaaza-7,10- methanocyclohepta [4,5]cycloocta[1,2,3-de] naphthalen-13- yl)oxy)methyl) cyclopropyl)methyl)- 3-methylpyrrolidin-3-ol | 6.69 (s, 1H), 6.46- 6.40 (m,1H), 4.41- 3.45 (m, 9H), 2.89- 2.71 (m, 6H), 2.42 (s, 3H), 2.09-1.62 (m, 8H), 0.76-0.60 (m, 4H) | 658.5 |

(mixture of two
isomers)

| | | | | |
|---|---|---|---|---|
| 94 | 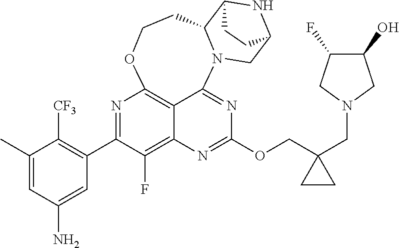 [SMILES: Cc1cc(N)cc(-c2nc3c4c(nc(OCC5(CN6C[C@H](O)[C@@H](F)C6)CC5)nc4c2F)N2C[C@H]4CC[C@H](N4)[C@H]2CCO3)c1C(F)(F)F] | trans-1-((1-((((6aR, 7S,10R)-2-(5- amino-3-methyl-2- (trifluoromethyl) phenyl)-1-fluoro- 5,6,6a,7,8,9,10,11- octahydro-4-oxa- 3,11a,12,14,15- pentaaza-7,10- methanocyclohepta [4,5]cycloocta[1,2,3-de] naphthalen-13-yl)oxy) methyl)cyclopropyl) methyl)-4- fluoropyrrolidin-3-ol | 8.39 (s, 1H), 6.70 (s, 1H), 6.44 (s, 1H), 4.91-4.89 (m, 2H), 4.79-4.78 (m, 1H), 4.57-4.53 (m, 1H), 4.46-4.24 (m, 5H), 4.10-3.99 (m, 3H), 3.82-3.72 (m, 1H), 3.13-2.94 (m, 1H), 2.80-2.78 (m, 1H), 2.59-2.55 (m, 2H), 2.43-2.42 (m, 4H), 2.30-2.01 (m, 5H), 0.77-0.73 (m, 2H), 0.62-0.57 (m, 2H). | 662.54 |
| | | formate | | |

| | | | | |
|---|---|---|---|---|
| 95 | 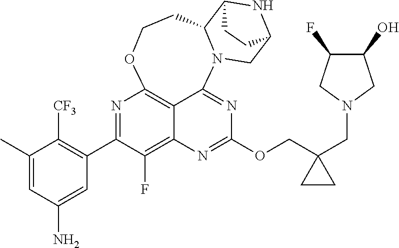 [SMILES: Cc1cc(N)cc(-c2nc3c4c(nc(OCC5(CN6C[C@@H](F)[C@@H](O)C6)CC5)nc4c2F)N2C[C@H]4CC[C@H](N4)[C@H]2CCO3)c1C(F)(F)F] | cis-1-((1-((((6aR, 7S,10R)-2-(5- amino-3-methyl-2- (trifluoromethyl) phenyl)-1-fluoro- 5,6,6a,7,8,9,10,11- octahydro-4-oxa- 3,11a,12,14,15- pentaaza-7,10- methanocyclohepta [4,5]cycloocta[1,2,3-de] naphthalen-13-yl)oxy) methyl)cyclopropyl) methyl)-4- fluoropyrrolidin-3-ol | 6.69 (s, 1H), 6.44 (s, 1H), 4.95-4.93 (m, 1H), 4.46-4.32 (m, 5H), 4.22-4.15 (m, 1H), 4.04-3.81 (m, 4H), 3.24-3.15 (m, 1H), 3.08-3.04 (m, 1H), 2.96-2.88 (m, 1H), 2.72-2.63 (m, 3H), 2.43-2.42 (m, 3H), 2.30-1.68 (m, 6H), 0.73-0.71 (m, 2H), 0.58-0.56 (m, 2H). | 662.53 |
| | | formate | | |

| | | | | |
|---|---|---|---|---|
| 96 | 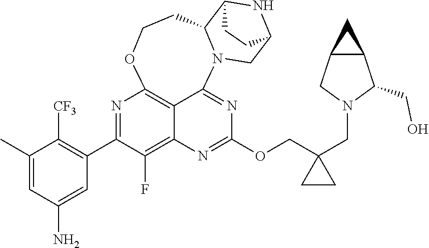 [SMILES: Cc1cc(N)cc(-c2nc3c4c(nc(OCC5(CN6C[C@H]7C[C@H]7[C@@H]6CO)CC5)nc4c2F)N2C[C@H]4CC[C@H](N4)[C@H]2CCO3)c1C(F)(F)F] | ((1R,2R,5S)-3- ((1-((((6aR,7S,10R)- 2-(5-amino-3- methyl-2-(trifluoromethyl) phenyl)-1-fluoro- 5,6,6a,7,8,9,10,11- octahydro-4-oxa- 3,11a,12,14,15- pentaaza-7,10- methanocyclohepta [4,5]cycloocta[1,2,3-de] naphthalen-13-yl)oxy) methyl)cyclopropyl) methyl)-3-azabicyclo [3.1.0]hexan-2-yl) methanol | 6.68 (s, 1H), 6.44 (d, J = 18.7 Hz, 1H), 4.42-4.21 (m, 5H), 4.12-4.05 (m, 1H), 3.74-3.68 (m, 1H), 3.61-3.53 (m, 2H), 3.08-2.98 (m, 1H), 2.90-2.76 (m, 3H), 2.63-2.55 (m, 1H), 2.42 (s, 3H), 2.15- 1.73 (m, 8H), 1.45- 1.28 (m, 4H), 1.00- 0.86 (m, 2H), 0.65- 0.55 (m, 4H), 0.46- 0.4 (m, 2H). | 670.5 |

| | | | | |
|---|---|---|---|---|
| 97 | 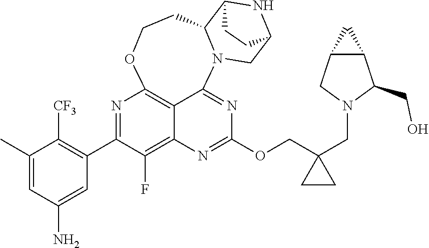 [SMILES: Cc1cc(N)cc(-c2nc3c4c(nc(OCC5(CN6C[C@@H]7C[C@@H]7[C@H]6CO)CC5)nc4c2F)N2C[C@H]4CC[C@H](N4)[C@H]2CCO3)c1C(F)(F)F] | ((1S,2S,5R)-3-((1- ((((6aR,7S,10R)-2-(5- amino-3-methyl-2- (trifluoromethyl) phenyl)-1-fluoro- 5,6,6a,7,8,9,10,11- octahydro-4-oxa- 3,11a,12,14,15- pentaaza-7,10- methanocyclohepta [4,5]cycloocta,[1,2,3-de] naphthalen-13-yl)oxy) methyl)cyclopropyl) methyl)-3-azabicyclo [3.1.0]hexan-2-yl) | 6.68 (s, 1H), 6.43 (d, J = 14.5 Hz, 1H), 4.47-4.21 (m, 5H), 4.14-4.03 (m, 1H), 3.72-3.67 (m, 1H), 3.62-3.54 (m, 2H), 3.05-2.98 (m, 1H), 2.92-2.79 (m, 3H), 2.58-2.47 (m, 1H), 2.42 (s, 3H), 2.17- 1.69 (m, 8H), 1.52- 1.22 (m, 4H), 1.20- 0.89 (m, 2H), 0.65- 0.5 (m, 4H), 0.48- 0.4 (m, 2H). | 670.5 |
| | | methanol | | |

| | | | | |
|---|---|---|---|---|
| 98 | 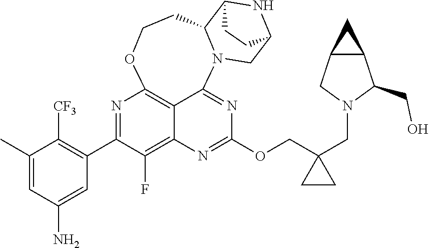 [SMILES: Cc1cc(N)cc(-c2nc3c4c(nc(OCC5(CN6C[C@H]7C[C@H]7[C@H]6CO)CC5)nc4c2F)N2C[C@H]4CC[C@H](N4)[C@H]2CCO3)c1C(F)(F)F] | ((1R,2S,5S)-3-((1- ((((6aR,7S,10R)- 2-(5-amino-3-methyl-2- (trifluoromethyl) phenyl)-1-fluoro- 5,6,6a,7,8,9,10,11- octahydro-4-oxa- 3,11a,12,14,15- pentaaza-7,10- methanocyclohepta [4,5]cycloocta[1,2,3-de] naphthalen-13-yl)oxy) methyl)cyclopropyl) methyl)-3-azabicyclo [3.1.0]hexan-2-yl) | 6.68 (s, 1H), 6.44 (d, J = 16.1 Hz, 1H), 4.85-4.78 (m, 1H), 4.48-3.95 (m, 5H), 3.85-3.74 (m, 1H), 3.71-3.65 (m, 1H), 3.64-3.51 (m, 2H), 3.48-3.37 (m, 1H), 3.21-3.08 (m, 2H), 2.68-2.58 (m, 1H), 2.42 (s, 3H), 2.36- 2.30 (m, 1H), 2.16- 1.72 (m, 7H), 1.59- 1.51 (m, 2H), 1.45- 1.27 (m, 2H), 1.19- | 670.5 |
| | | methanol | 0.75 (m, 2H), 0.64- | |
| | | | 0.56 (m, 2H), 0.54- | |
| | | | 0.47 (m, 2H), 0.37- | |
| | | | 0.32 (m, 1H), 0.25- | |
| | | | 0.18 (m, 1H). | |
| 99 | 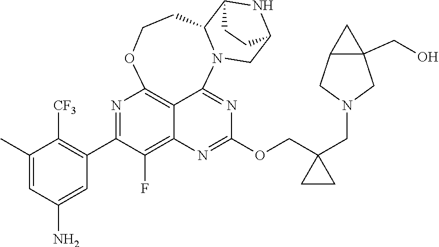 [SMILES: Cc1cc(N)cc(-c2nc3c4c(nc(OCC5(CN6CC7CC7(CO)C6)CC5)nc4c2F)N2C[C@H]4CC[C@H](N4)[C@H]2CCO3)c1C(F)(F)F] | (3-((1-((((6aR,7S,10R)- 2-(5-amino-3-methyl- 2-(trifluoromethyl) phenyl)-1-fluoro- 5,6,6a,7,8,9,10,11- octahydro-4-oxa- 3,11a,12,14,15- pentaaza-7,10- methanocyclohepta [4,5]cycloocta [1,2,3-de]naphthalen- 13-yl)oxy)methyl) cyclopropyl)methyl)- 3-azabicyclo[3.1.0] hexan-1-yl)methanol | 6.68 (s, 1H), 6.43 (d, J = 20.0 Hz, 1H), 4.47-4.20 (m, 5H), 4.12-3.88 (m, 2H), 3.73-3.47 (m, 4H), 3.15-3.08 (m, 1H), 3.08-2.97 (m, 1H), 2.51 (s, 1H), 2.45- 2.34 (m, 5H), 2.14- 1.72 (m, 7H), 1.49- 0.95 (m, 5H), 0.94- 0.85 (m, 1H), 0.70- 0.60 (m, 2H), 0.51- 0.37 (m, 3H). | 670.5 |
| | (mixture of two | | | |
| | isomers) | | | |
| 100 | 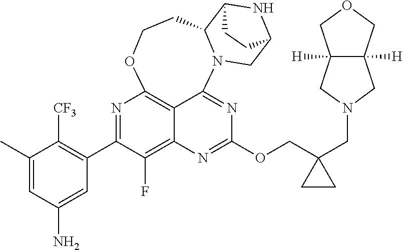 [SMILES: Cc1cc(N)cc(-c2nc3c4c(nc(OCC5(CN6C[C@H]7COC[C@H]7C6)CC5)nc4c2F)N2C[C@H]4CC[C@H](N4)[C@H]2CCO3)c1C(F)(F)F] | 3-((6aR,7S,10R)-1- fluoro-13-((1-(((3aR,6aS)- tetrahydro-1H-furo[3,4-c] pyrrol-5(3H)-yl)methyl) cyclopropyl)methoxy)- 5,6,6a,7,8,9,10,11- octahydro-4-oxa- 3,11a,12,14,15- pentaaza-7,10- methanocyclohepta [4,5]cycloocta [1,2,3-de]naphthalen- 2-yl)-5-methyl- 4-(trifluoromethyl) aniline | 6.69 (s, 1H), 6.46- 6.41 (m,1H), 4.44- 3.62 (m, 13H), 3.09- 2.65 (m, 8H), 2.42 (s, 3H), 2.12-1.89 (m, 6H), 0.78-0.63 (m, 4H) | 670.5 |
| 101 | 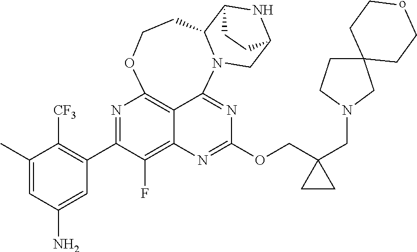 | 3-((6aR,7S,10R)- 13-((1-((8-oxa-2- azaspiro[4.5]decan-2- yl)methyl)cyclopropyl) methoxy)-1- fluoro-5,6,6a, 7,8,9,10,11- octahydro-4-oxa- 3,11a,12,14,15- pentaaza[7,10] methanocyclohepta [4,5]cycloocta[1,2,3- de]naphthalen-2- yl)-5-methyl-4- (trifluoromethyl) aniline formate | 6.69 (s, 1H), 6.45- 6.40 (m, 1H), 4.48- 4.28 (m, 5H), 4.10- 3.97 (m, 2H), 3.81- 3.63 (m, 6H), 3.26- 3.06 (m, 6H), 2.43- 2.42 (m, 3H), 2.11- 1.91 (m, 8H), 1.72- 1.59 (m, 4H), 0.88- 0.85 (m, 2H), 0.76- 0.74 (m, 2H). | 698.6 |
| 102 | 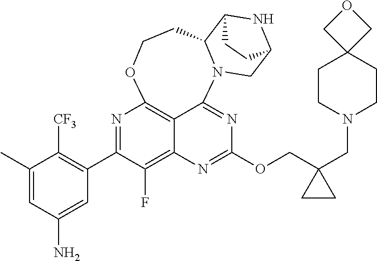 [SMILES: Cc1cc(N)cc(-c2nc3c4c(nc(OCC5(CN6CCC7(CC6)COC7)CC5)nc4c2F)N2C[C@H]4CC[C@H](N4)[C@H]2CCO3)c1C(F)(F)F] | 3-((6aR,7S,10R)- 13-((1-((2-oxa-7- azaspiro[3.5]nonan-7- yl)methyl)cyclopropyl) methoxy)-1- fluoro-5,6,6a,7, 8,9,10,11-octahydro- 4-oxa-3,11a,12,14,15- pentaaza[7,10] methanocyclohepta [4,5]cycloocta [1,2,3-de] naphthalen-2-yl)- 5-methyl-4- (trifluoromethyl)aniline | 6.69(s, 1H), 6.45- 6.39 (m,1H), 4.42- 3.69 (m, 13H), 2.80- 2.61 (m, 6H), 2.42 (s, 3H), 2.15-1.72 (m, 10H), 0.78-0.58 (m, 4H). | 684.5 |
| 103 | 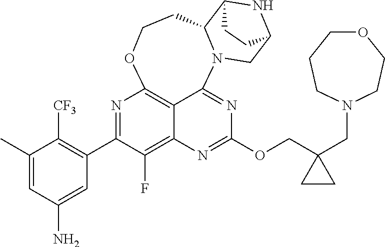 [SMILES: Cc1cc(N)cc(-c2nc3c4c(nc(OCC5(CN6CCCOCC6)CC5)nc4c2F)N2C[C@H]4CC[C@H](N4)[C@H]2CCO3)c1C(F)(F)F] | 3-((6aR,7S,10R)-13-- ((1-((1,4-oxazepan-4- yl)methyl)cyclopropyl) methoxy)-1- fluoro-5,6,6a,7,8,9,10,11- octahydro-4-oxa-3,11a, 12,14,15-pentaaza-7,10- methanocyclohepta [4,5]cycloocta [1,2,3-de]naphthalen- 2-yl)-5-methyl- 4-(trifluoromethyl)aniline | 6.70 (s, 1H), 6.47- 6.37 (m, 1H), 4.47- 3.75 (m, 13H), 3.37- 2.96 (m, 7H), 2.42 (s, 3H), 2.42-1.84 (m, 7H), 0.85-0.69 (m, 4H) | 658.5 |
| 104 | 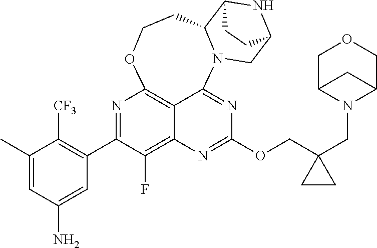 [SMILES: Cc1cc(N)cc(-c2nc3c4c(nc(OCC5(CN6C7COCC6C7)CC5)nc4c2F)N2C[C@H]4CC[C@H](N4)[C@H]2CCO3)c1C(F)(F)F] | 3-((6aR,7S,10R)- 13-((1-((3-oxa-6- azabicyclo[3.1.1] heptan-6- yl)methyl)cyclopropyl) methoxy)-1-fluoro- 5,6,6a,7,8,9,10,11- octahydro-4-oxa- 3,11a,12,14,15- pentaaza-7,10- methanocyclohepta [4,5]cycloocta [1,2,3-de]naphthalen- 2-yl)-5-methyl- 4-(trifluoromethyl) | 6.69 (s, 1H), 6.45- 6.41 (m, 1H), 4.53- 3.932 (m, 14H), 3.81- 3.72 (m, 2H), 3.09 (s, 1H), 2.43-2.42 (m, 3H), 2.14-1.87 (m, 8H), 0.88-0.83 (m, 4H). | 656.55 |
| | | aniline formate | | |
| 105 | 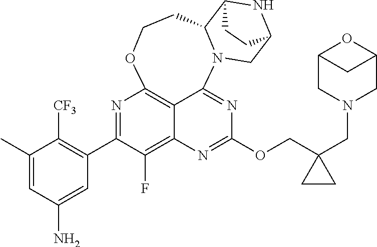 [SMILES: Cc1cc(N)cc(-c2nc3c4c(nc(OCC5(CN6CC7CC(C6)O7)CC5)nc4c2F)N2C[C@H]4CC[C@H](N4)[C@H]2CCO3)c1C(F)(F)F] | 3-((6aR,7S,10R)-13- ((1-((6-oxa-3-azabicyclo [3.1.1]heptan-3- yl)methyl)cyclopropyl) methoxy)-1-fluoro- 5,6,6a,7,8,9,10,11- octahydro-4-oxa- 3,11a,12,14,15- pentaaza-7,10- methanocyclohepta [4,5]cycloocta [1,2,3-de]naphthalen- 2-yl)-5-methyl- 4-(trifluoromethyl)aniline | 6.69 (s, 1H), 6.44 (s, 1H), 4.48-4.43 (m, 4H), 4.31-3.83 (m, 7H), 3.22-3.20 (m, 2H), 2.95-2.91 (m, 1H), 2.73-2.63 (m, 4H), 2.43-2.42 (m, 3H), 2.28-1.79 (m, 7H), 0.72-0.70 (m, 2H), 0.55-1.53 (m, 2H). | 656.53 |
| 106 | 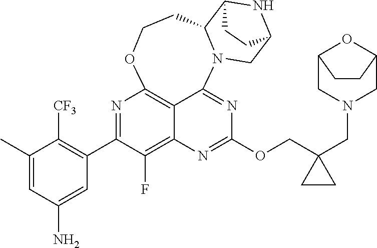 [SMILES: Cc1cc(N)cc(-c2nc3c4c(nc(OCC5(CN6CC7CCC(C6)O7)CC5)nc4c2F)N2C[C@H]4CC[C@H](N4)[C@H]2CCO3)c1C(F)(F)F] | 3-((6aR,7S,10R)- 13-((1-(((1R,5S)- 8-oxa-3-azabicyclo [3.2.1]octan-3- yl)methyl)cyclopropyl) methoxy)-1- fluoro-5,6,6a, 7,8,9,10,11- octahydro-4-oxa- 3,11a,12,14,15- pentaaza-7,10- methanocyclohepta [4,5]cycloocta [1,2,3-de]naphthalen- 2-yl)-5-methyl- | 6.70 (s, 1H), 6.47- 6.43 (m, 1H), 4.47- 3.60 (m, 16H), 3.00- 2.88 (m, 1H), 2.42 (s. 3H), 2.10-1.98 (m, 10H), 0.85-0.75 (m, 4H) | 670.5 |
| | | 4-(trifluoromethyl)aniline | | |
| 107 | 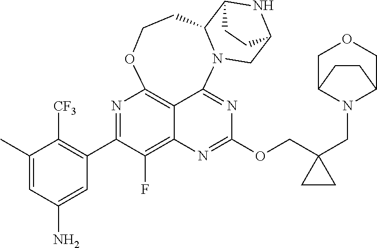 [SMILES: Cc1cc(N)cc(-c2nc3c4c(nc(OCC5(CN6C7CCC6COC7)CC5)nc4c2F)N2C[C@H]4CC[C@H](N4)[C@H]2CCO3)c1C(F)(F)F] | 3-((6aR,7S,10R)- 13-((1-(((1R,5S)- 3-oxa-8-azabicyclo [3.2.1]octan-8- yl)methyl)cyclopropyl) methoxy)-1-fluoro- 5,6,6a,7,8,9,10,11- octahydro-4-oxa- 3,11a,12,14,15- pentaaza-7,10- methanocyclohepta [4,5]cycloocta [1,2,3-de]naphthalen- 2-yl)-5-methyl- 4-(trifluoromethyl)aniline | 6.70 (s, 1H), 6.47- 6.43 (m, 1H), 4.52- 3.65 (m, 16H), 2.96- 2.79 (m, 2H), 2.42 (s 3H), 2.20-1.91 (m, 9H), 0.83-0.72 (m, 4H) | 670.5 |
| 108 | 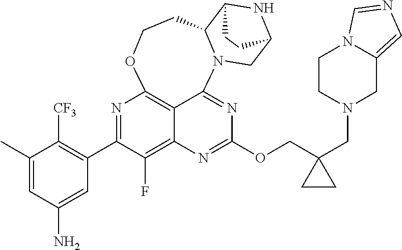 [SMILES: Cc1cc(N)cc(-c2nc3c4c(nc(OCC5(CN6CCn7cncc7C6)CC5)nc4c2F)N2C[C@H]4CC[C@H](N4)[C@H]2CCO3)c1C(F)(F)F] | 3-((6aR,7S,10R)- 13-((1-((5,6- dihydroimidazo [1,5-a]pyrazin-7(8H)- yl)methyl)cyclopropyl) methoxy)-1- fluoro-5,6,6a,7,8,9, 10,11-octahydro-4-oxa- 3,11a,12,14,15- pentaaza-7,10- methanocyclohepta [4,5]cycloocta [1,2,3-de]naphthalen- 2-yl)-5-methyl- 4-(trifluoromethyl)aniline | 7.49(s,1 H), 6.69- 6.66(m, 2H), 6.48- 6.47 (m,1H), 4.49- 3.63(m, 13H), 2.91- 2.55 (m, 2H), 2.42 (s, 3H), 2.18-1.83 (m, 6H), 0.80-0.53(m, 4H). | 680.5 |
| 109 | 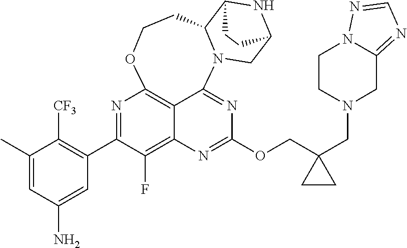 [SMILES: Cc1cc(N)cc(-c2nc3c4c(nc(OCC5(CN6CCn7ncnc7C6)CC5)nc4c2F)N2C[C@H]4CC[C@H](N4)[C@H]2CCO3)c1C(F)(F)F] | 3-((6aR,7S,10R)- 13-((1-((5,6- dihydro-[1,2,4]triazolo[1,5- a]pyrazin-7(8H)- yl)methyl)cyclopropyl) methoxy)-1- fluoro-5,6,6a,7,8,9,10,11- octahydro-4-oxa-3,11a, 12,14,15-pentaaza-7,10- methanocyclohepta [4,5]cycloocta [1,2,3-de]naphthalen- 2-yl)-5-methyl- 4-(trifluoromethyl)aniline | 7.85(s, 1H), 6.70 (s, 1H), 6.45-6.41 (m, 1H), 4.49-3.82 (m, 16H), 2.86-2.61 (m, 2H), 2.42 (s, 3H), 2.21-1.75 (m, 6H), 0.87-0.84 (m, 4H). | 682.5 |

### Example 110: 3-((6aR,7S,10R)-1-fluoro-13-((1-((4-methoxypiperidin-1-yl)methyl)cyclopropyl)methoxy)-5,6,6a,7,8,9,10,11-octahydro-4-oxa-3,11a,12,14,15-pentaaza-7,10-methanocyclohepta[4,5]cycloocta[1,2,3-de]naphthalen-2-yl)-5-methyl-4-(trifluoromethyl)aniline

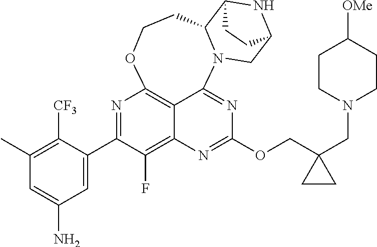 [SMILES: COC1CCN(CC2(COc3nc4c5c(nc(-c6cc(N)cc(C)c6C(F)(F)F)c(F)c5n3)OCC[C@@H]3[C@@H]5CC[C@H](CN43)N5)CC2)CC1]

### Synthetic Route

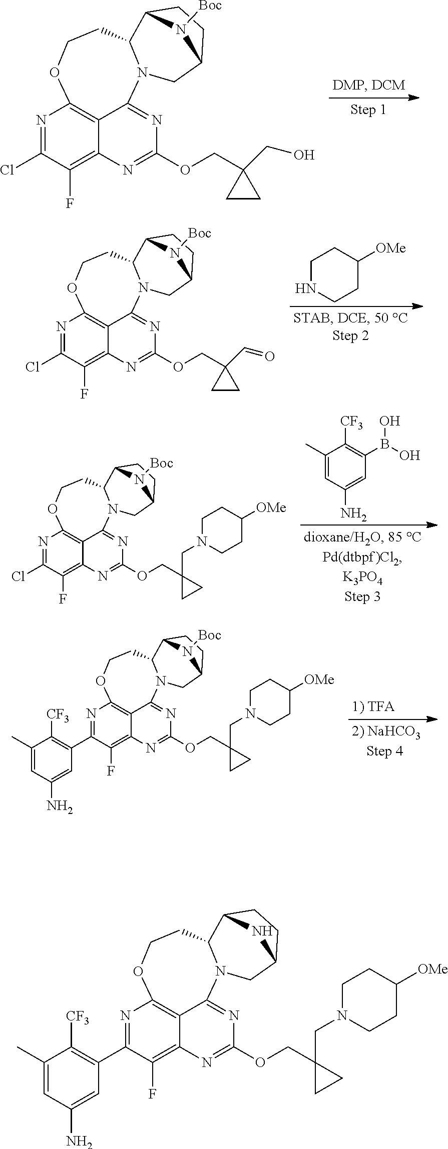

### Step 1: tert-butyl (6aR,7S,10R)-2-chloro-1-fluoro-13-((1-formylcyclopropyl)methoxy)-5,6,6a,7,8,9,10,11-octahydro-4-oxa-3,11a,12,14,15-pentaaza-7,10-methanocyclohepta[4,5]cycloocta[1,2,3-de]naphthalene-15-carboxylate

To a solution of tert-butyl (6aR,7S,10R)-2-chloro-1-fluoro-13-((1-(hydroxymethyl)cyclopropyl)methoxy)-5,6,6a,7,8,9,10,11-octahydro-4-oxa-3,11a,12,14,15-pentaaza-7,10-methanocyclohepta[4,5]cycloocta[1,2,3-de]naphthalene-15-carboxylate (0.70 g, 1.3 mmol) in DCM (60 mL) was added Dess-Martin Periodinane (0.66 g, 0.42 mmol) at 0° C. The mixture was stirred at rt for 30 minutes. The reaction was then quenched by addition of saturated sodium thiosulfate solution (30 mL). The mixture was extracted with DCM (20 mL) for 3 times. The combined organic phase was washed with brine (50 mL), dried over Na₂SO₄, and filtered through Celite. The filtrate was concentrated in vacuo and purified by silica gel column chromatography (PE/EtOAc=3/1) to give the title product (0.57 g). MS (ESI, m/e) [M+H]⁺ 534.3.

### Step 2: tert-butyl (6aR,7S,10R)-2-chloro-1-fluoro-13-((1-((4-methoxypiperidin-1-yl)methyl)cyclopropyl)methoxy)-5,6,6a,7,8,9,10,11-octahydro-4-oxa-3,11a,12,14,15-pentaaza-7,10-methanocyclohepta[4,5]cycloocta[1,2,3-de]naphthalene-15-carboxylate

To a solution of tert-butyl (6aR,7S,10R)-2-chloro-1-fluoro-13-((1-formylcyclopropyl)methoxy)-5,6,6a,7,8,9,10,11-octahydro-4-oxa-3,11a,12,14,15-pentaaza-7,10-methanocyclohepta[4,5]cycloocta[1,2,3-de]naphthalene-15-carboxylate (38 mg, 0.07 mmol) in DCE (5 mL) was added 4-methoxypiperidine (18 mg, 0.14 mmol) and acetic acid (2 drops). The resulting mixture was stirred at 50° C. for 6 hours, followed by addition of sodium triacetoxyborohydride (45 mg, 0.21 mmol). The mixture was further stirred at 50° C. for 16 hours. After the reaction was complete as indicated by TLC, the reaction was quenched by addition of saturated NaHCO₃ aqueous solution (10 mL). The mixture was extracted with EtOAc (20 mL) for 3 times. The combined organic phase was washed with brine (10 mL), dried over sodium sulfate and filtered through Celite. The filtrate was concentrated in vacuo, and purified by silica gel column chromatography (eluted with EtOAc) to give the title product (20 mg). MS (ESI, m/e) [M+H]⁺ 633.5.

### Step 3: tert-butyl (6aR,7S,10R)-2-(5-amino-3-methyl-2-(trifluoromethyl)phenyl)-1-fluoro-13-((1-((4-methoxypiperidin-1-yl)methyl)cyclopropyl)methoxy)-5,6,6a,7,8,9,10,11-octahydro-4-oxa-3,11a,12,14,15-pentaaza-7,10-methanocyclohepta[4,5]cycloocta[1,2,3-de]naphthalene-15-carboxylate

A mixture of tert-butyl (6aR,7S,10R)-2-chloro-1-fluoro-13-((1-((4-methoxypiperidin-1-yl)methyl)cyclopropyl)methoxy)-5,6,6a,7,8,9,10,11-octahydro-4-oxa-3,11a,12,14,15-pentaaza-7,10-methanocyclohepta[4,5]cycloocta[1,2,3-de]naphthalene-15-carboxylate (20 mg, 0.03 mmol), (5-amino-3-methyl-2-(trifluoromethyl)phenyl)boronic acid (25 mg, 0.11 mmol), potassium phosphate (33 mg, 0.16 mmol), dioxane/H₂O (5:1 ratio, 10 mL) and Pd(dtbpf)Cl₂ (6 mg, 0.009 mmol) was degassed by bubbling nitrogen through for 10 min. The resulting mixture was stirred at 85° C. for 2 hours. The mixture was then diluted with EtOAc, and filtered with Celite. The filtrate was concentration in vacuo, and purified by silica gel column chromatography (DCM/MeOH=10/1) to give the title product (18 mg). MS (ESI, m/e) [M+H]⁺ 772.5.

### Step 4: ((6aR,7S,10R)-1-fluoro-13-((1-((4-methoxypiperidin-1-yl)methyl)cyclopropyl)methoxy)-5,6,6a,7,8,9,10,11-octahydro-4-oxa-3,11a,12,14,15-pentaaza-7,10-methanocyclohepta[4,5]cycloocta[1,2,3-de]naphthalen-2-yl)-5-methyl-4-(trifluoromethyl)aniline

To a solution of tert-butyl (6aR,7S,10R)-2-(5-amino-3-methyl-2-(trifluoromethyl)phenyl)-1-fluoro-13-((1-((4-methoxypiperidin-1-yl)methyl)cyclopropyl)methoxy)-5,6,6a,7,8,9,10,11-octahydro-4-oxa-3,11a,12,14,15-pentaaza-7,10-methanocyclohepta[4,5]cycloocta[1,2,3-de]naphthalene-15-carboxylate (18 mg, 0.02 mmol) in DCM (10 mL) was added TFA (2 mL). The resulting mixture was stirred at rt for 2 hours, which was then concentrated in vacuo. The residue was diluted with NaHCO₃ aqueous solution and extracted with DCM (10 mL) 2 times. The combined DCM phase was dried by Na₂SO₄ and concentrated to give the crude residue which was further purified by Prep-HPLC (Column: Waters X-Select C18, 19*150 mm, 5 μm; Mobile Phase A: H2O (0.1% FA), Mobile Phase B: MeCN (0.1% FA); Flow Rate: 17 mL/min; Temperature: 25° C.; Detector: UV 254 nm and 214 nm; Gradient: 12 to 35% B within 1 min to 11 min) to give the title product (2.6 mg). ¹H NMR (500 MHz, CD₃OD) δ 6.70 (s, 1H), 6.44 (d, J=18.7 Hz, 1H), 4.67-3.68 (m, 9H), 3.46 (d, J=19.0 Hz, 1H), 3.35 (s, 3H), 3.05 (s, 3H), 2.42 (d, J=1.6 Hz, 3H), 2.28-1.57 (m, 10H), 0.90 (s, 2H), 0.76 (s, 2H). MS (ESI, m/e) [M+H]⁺ 672.6.

Examples 111-121: The following examples were prepared in a manner similar to that in Example 110 by replacing 4-methoxypiperidine with the corresponding alkyl amines.

| | | | | MS |
|---|---|---|---|---|
| | | | | (ESI, |
| | | | | m/e) |
| Ex- | | | 1H NMR (500 MHz, | [M + |
| ample | Structure | Compound Name | CD₃OD) δ | H]⁺ |
| 111 | 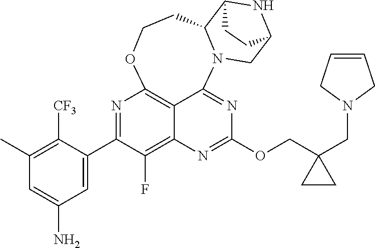 [SMILES: Cc1cc(N)cc(-c2nc3c4c(nc(OCC5(CN6CC=CC6)CC5)nc4c2F)N2C[C@H]4CC[C@H](N4)[C@H]2CCO3)c1C(F)(F)F] | 3-((6aR,7S,10R)- 13-((1-((2,5- dihydro-1H-pyrrol-1- yl)methyl)cyclopropyl )methoxy)- 1-fluoro-5,6,6a,7,8,9,10,11- octahydro-4-oxa- 3,11a,12,14,15- pentaaza-7,10- methanocyclohepta [4,5]cycloocta [1,2,3-de]naphthalen-2-yl)-5- methyl-4-(trifluoromethyl) aniline | 6.70 (s, 1H), 6.45-6.40 (m, 1H), 6.01-5.93 (m, 2H), 4.58-4.29 (m, 5H), 4.10-3.98 (m, 6H), 3.88-3.80 (m, 2H), 3.33-3.32 (m, 2H), 2.43-2.422 (m, 3H), 2.13-1.91 (m, 6H), 0.90-0.81 (m, 4H). | 626.6 |
| 112 | 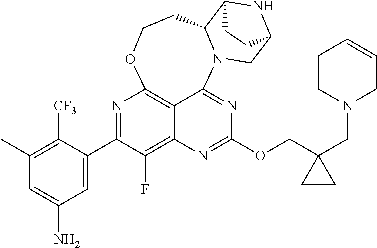 [SMILES: Cc1cc(N)cc(-c2nc3c4c(nc(OCC5(CN6CC=CCC6)CC5)nc4c2F)N2C[C@H]4CC[C@H](N4)[C@H]2CCO3)c1C(F)(F)F] | 3-((6aR,7S,10R)- 13-((1-((3,6- dihydropyridin-1(2H)- yl)methyl)cyclopropyl) methoxy)- 1-fluoro-5,6,6a, 7,8,9,10,11- octahydro-4-oxa- 3,11a,12,14,15- pentaaza-7,10- methanocyclohepta [4,5]cycloocta [1,2,3-de]naphthalen-2-yl)-5- methyl-4-(trifluoromethyl) aniline | 6.68 (s, 1H), 6.46-6.41 (m, 1H), 5.70-5.62 (m, 2H), 4.44-4.33 (m, 3H), 4.26 (s, 1H), 4.06-3.93 (m, 2H), 3.66 (s, 1H), 3.56-3.52 (m, 2H), 3.02 (s, 2H), 2.66-2.65 (m, 2H), 2.57-2.52 (m, 2H), 2.43-2.42 (m, 3H), 2.19-1.80 (m, 8H), 0.74-0.72 (m, 2H), 0.54-1.52 (m, 2H). | 640.6 |
| 113 | 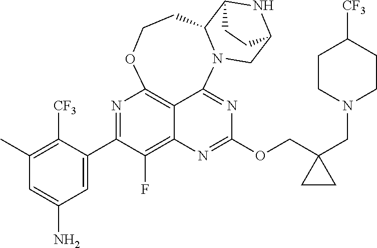 [SMILES: Cc1cc(N)cc(-c2nc3c4c(nc(OCC5(CN6CCC(C(F)(F)F)CC6)CC5)nc4c2F)N2C[C@H]4CC[C@H](N4)[C@H]2CCO3)c1C(F)(F)F] | 3-((6aR,7S,10R)- 1-fluoro-13-((1- ((4-(trifluoromethyl) piperidin-1- yl)methyl)cyclopropyl) methoxy)- 5,6,6a,7,8,9,10,11- octahydro-4- oxa-3,11a,12,14,15-pentaaza- 7,10- methanocyclohepta [4,5]cycloocta [1,2,3-de]naphthalen-2-yl)-5- methyl-4-(trifluoromethyl) aniline | 6.70 (s, 1H), 6.44 (s, 1H), 4.60-3.4 (m, 8H), 3.45-3.40 (m, 2H), 2.69 (s, 2H), 2.53- 1.80 (m, 13H), 1.64 (d, J = 10.8 Hz, 2H), 0.81 (s, 2H), 0.63 (s, 2H). (2H obscured by solvent peak) | 710.5 |
| 114 | 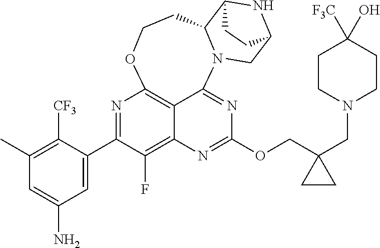 [SMILES: Cc1cc(N)cc(-c2nc3c4c(nc(OCC5(CN6CCC(O)(C(F)(F)F)CC6)CC5)nc4c2F)N2C[C@H]4CC[C@H](N4)[C@H]2CCO3)c1C(F)(F)F] | 1-((1-((((6aR,7S,10R)-2-(5- amino-3-methyl-2- (trifluoromethyl)phenyl)-1- fluoro-5,6,6a,7,8,9,10,11- octahydro-4-oxa- 3,11a,12,14,15- pentaaza-7,10- methanocyclohepta [4,5]cycloocta [1,2,3-de]naphthalen- 13-yl)oxy) methyl)cyclopropyl) methyl)- 4-(trifluoromethyl) piperidin-4-ol | 6.70 (s, 1H), 6.44 (s, 1H), 4.60-3.62 (m, 9H), 3.28-3.19 (m, 1H), 2.65 (s, 4H), 2.51- 1.59 (m, 13H), 0.83 (s, 2H), 0.66 (s, 2H). (1H obscured by solvent peak) | 726.5 |
| 115 | 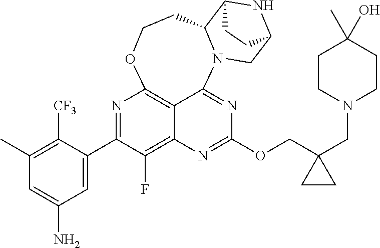 [SMILES: Cc1cc(N)cc(-c2nc3c4c(nc(OCC5(CN6CCC(C)(O)CC6)CC5)nc4c2F)N2C[C@H]4CC[C@H](N4)[C@H]2CCO3)c1C(F)(F)F] | 1-((1-((((6aR,7S, 10R)-2-(5-amino-3- methyl-2- (trifluoromethyl) phenyl)-1-fluoro- 5,6,6a,7,8,9,10,11- octahydro-4-oxa-3, 11a,12,14,15- pentaaza-7,10- methanocyclohepta [4,5]cycloocta[1,2,3-de] naphthalen-13- yl)oxy)methyl) cyclopropyl)methyl)- 4-methylpiperidin-4-ol | 6.70 (s, 1H), 6.43 (d, J = 18.4 Hz, 1H), 4.62- 4.21 (m, 5H), 4.12 (s, 2H), 3.83 (d, J = 36.9 Hz, 2H), 3.65-3.39 (m, 2H), 3.25-3.01 (m, 3H), 2.42 (d, J = 1.9 Hz, 3H), 2.28- 1.60 (m, 10H), 1.29 (s, 3H), 0.93 (d, J = 19.6 Hz, 2H), 0.86 (d, J = 20.9 Hz, 2H). (1H obscured by solvent peak) | 672.6 |
| 116 | 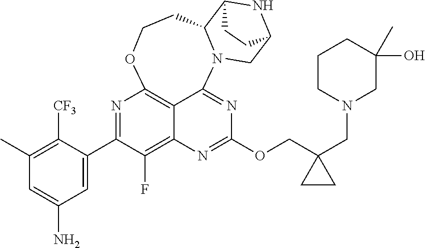 [SMILES: Cc1cc(N)cc(-c2nc3c4c(nc(OCC5(CN6CCCC(C)(O)C6)CC5)nc4c2F)N2C[C@H]4CC[C@H](N4)[C@H]2CCO3)c1C(F)(F)F] | 1-((1-((((6aR,7S, 10R)-2-(5- amino-3-methyl-2- (trifluoromethyl) phenyl)-1- fluoro-5,6,6a,7,8,9,10,11- octahydro-4-oxa- 3,11a,12,14,15- pentaaza-7,10- methanocyclohepta [4,5]cycloocta [1,2,3-de]naphthalen-13- yl)oxy)methyl) cyclopropyl)methyl)- 3-methylpiperidin-3-ol | 6.70 (s, 1H), 6.43 (d, J = 27.5 Hz, 1H), 4.64 (d, J = 11.2 Hz, 1H), 4.49- 3.50 (m, 9H), 3.31 (s, 1H), 3.02-2.65 (m, 3H), 2.42 (d, J = 1.8 Hz, 3H), 2.30-1.65 (m, 9H), 1.60-1.46 (m, 1H), 1.31 (s, 3H), 1.03 (s, 1H), 0.87 (s, 2H), 0.70 (s, 1H). (2H obscured by solvent peak) | 672.6 |
| | (mixture of two | | | |
| | isomers) | | | |
| 117 | 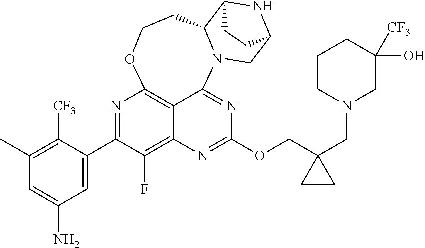 [SMILES: Cc1cc(N)cc(-c2nc3c4c(nc(OCC5(CN6CCCC(O)(C(F)(F)F)C6)CC5)nc4c2F)N2C[C@H]4CC[C@H](N4)[C@H]2CCO3)c1C(F)(F)F] | 1-((1-((((6aR,7S,10R)-2-(5- amino-3-methyl-2- (trifluoromethyl)phenyl)-1- fluoro-5,6,6a,7,8,9,10,11- octahydro-4-oxa- 3,11a,12,14,15- pentaaza-7,10- methanocyclohepta [4,5]cycloocta [1,2,3-de]naphthalen-13- yl)oxy)methyl) cyclopropyl)methyl)- 3-(trifluoromethyl) piperidin-3-ol | 6.70 (s, 1H), 6.44 (s, 1H), 4.63-3.72 (m, 8H), 3.05 (s, 1H), 2.82 (s, 1H), 2.42 (d, J = 2.1 Hz, 6H), 2.19 (s, 2H), 1.99 (d, J = 34.7 Hz, 4H), 1.80 (d, J = 12.5 Hz, 1H), 1.71-1.53 (m, 2H), 0.79 (d, J = 36.4 Hz, 2H), 0.56 (s, 2H). (3H obscured by solvent peak) | 726.5 |
| | (mixture of two | | | |
| | isomers) | | | |
| 118 | 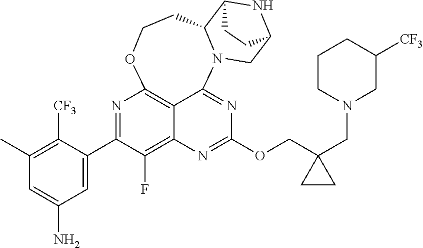 [SMILES: Cc1cc(N)cc(-c2nc3c4c(nc(OCC5(CN6CCCC(C(F)(F)F)C6)CC5)nc4c2F)N2C[C@H]4CC[C@H](N4)[C@H]2CCO3)c1C(F)(F)F] | 3-((6aR,7S,10R)- 1-fluoro-13-((1- ((3-(trifluoromethyl) piperidin-1-yl)methyl) cyclopropyl)methoxy)- 5,6,6a,7,8,9,10,11- octahydro-4- oxa-3,11a,12,14,15- pentaaza-7,10- methanocyclohepta [4,5]cycloocta [1,2,3-de]naphthalen- 2-yl)-5-methyl-4- (trifluoromethyl)aniline | 6.69 (s, 1H), 6.44 (s, 1H), 4.41-4.31 (m, 4H), 4.01-3.95 (m, 4H), 3.18 (s, 1H), 3.05 (s, 1H), 2.62-1.82 (m, 14H), 1.73 (d, J = 13.8 Hz, 1H), 1.50 (d, J = 12.8 Hz, 1H), 1.37- 1.22 (m, 1H), 0.73 (s, 2H), 0.52 (s, 2H). (2 H obscured by solvent peak) | 710.5 |
| | (mixture of two | | | |
| | isomers) | | | |
| 119 | 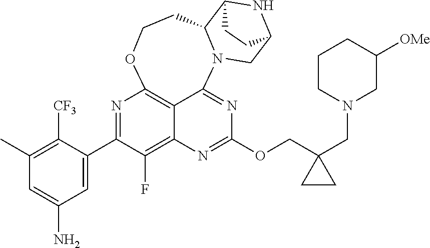 [SMILES: COC1CCCN(CC2(COc3nc4c5c(nc(-c6cc(N)cc(C)c6C(F)(F)F)c(F)c5n3)OCC[C@@H]3[C@@H]5CC[C@H](CN43)N5)CC2)C1] | 3-((6aR,7S,10R)- 1-fluoro-13-((1- ((3-methoxypiperidin-1- yl)methyl)cyclopropyl) methoxy)- 5,6,6a,7,8,9,10,11- octahydro-4-oxa- 3,11a,12,14,15- pentaaza-7,10- methanocyclohepta [4,5]cycloocta[1,2,3-de] naphthalen-2-yl)-5- methyl-4-(trifluoromethyl) aniline | 6.70 (s, 1H), 6.44 (s, 1H), 4.65-3.35 (m, 15H), 3.21-2.76 (m, 4H), 2.42 (d, J = 2.0 Hz, 3H), 2.03-1.69 (m, 10H), 1.08-0.61 (m, 4H) | 672.6 |
| | (mixture of two | | | |
| | isomers) | | | |
| 120 | 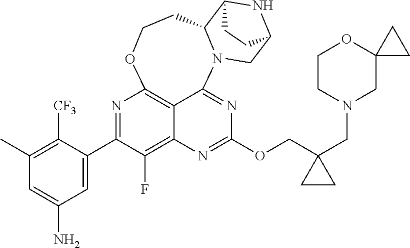 [SMILES: Cc1cc(N)cc(-c2nc3c4c(nc(OCC5(CN6CCOC7(CC7)C6)CC5)nc4c2F)N2C[C@H]4CC[C@H](N4)[C@H]2CCO3)c1C(F)(F)F] | 3-((6aR,7S,10R)- 13-((1-((4-oxa- 7-azaspiro[2.5]octan-7- yl)methyl)cyclopropyl) methoxy)-1-fluoro- 5,6,6a,7,8,9,10,11- octahydro-4-oxa- 3,11a,12,14,15- pentaaza[7,10] methanocyclohepta [4,5]cycloocta[1,2,3- de]naphthalen-2- yl)-5-methyl-4- (trifluoromethyl)aniline | 6.70 (s, 1H), 6.44 (s, 1H), 4.48-4.32 (m, 5H), 4.19-3.64 (m, 6H), 2.69 (d, J = 42.8 Hz, 6H), 2.52-1.79 (m, 9H), 0.76-0.59 (m, 8H) | 670.5 |
| 121 | 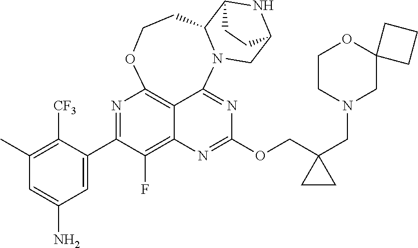 [SMILES: Cc1cc(N)cc(-c2nc3c4c(nc(OCC5(CN6CCOC7(CCC7)C6)CC5)nc4c2F)N2C[C@H]4CC[C@H](N4)[C@H]2CCO3)c1C(F)(F)F] | 3-((6aR,7S,10R)- 13-((1-((5-oxa-8- azaspiro[3.5]nonan-8- yl)methyl)cyclopropyl) methoxy)-1-fluoro- 5,6,6a,7,8,9,10,11- octahydro-4-oxa- 3,11a,12,14,15- pentaaza[7,10] methanocyclohepta [4,5]cycloocta[1,2,3- de]naphthalen-2-yl)- 5-methyl-4- (trifluoromethyl)aniline | 6.70 (s, 1H), 6.44 (s, 1H), 4.70-3.51 (m, 11H), 2.66 (s, 6H), 2.43 (d, J = 2.1 Hz, 4H), 2.27-1.71 (m, 10H), 1.67-1.51 (m, 1H), 0.80 (s, 2H), 0.61 (s, 2H | 684.6 |
| 122 | 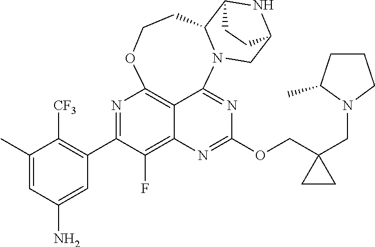 [SMILES: Cc1cc(N)cc(-c2nc3c4c(nc(OCC5(CN6CCC[C@H]6C)CC5)nc4c2F)N2C[C@H]4CC[C@H](N4)[C@H]2CCO3)c1C(F)(F)F] | 3-((6aR,7S,10R)- 1-fluoro-13-((1-(((R)-2- methylpyrrolidin-1- yl)methyl)cyclopropyl) methoxy)-5,6,6a,7,8,9, 10,11-octahydro-4- oxa-3,11a,12,14,15- pentaaza-7,10- methanocyclohepta [4,5]cycloocta [1,2,3-de]naphthalen- 2-yl)-5-methyl-4- (trifluoromethyl)aniline | 6.70 (s, 1H), 6.46-6.42 (m, 1H), 4.64 (s, 1H), 4.35-4.22 (m, 6H), 3.94-3.72 (m, 6H), 2.78 (s, 1H), 2.42 (s, 3H), 2.28-1.73 (m, 10H), 1.41 (s, 3H), 0.92-0.76 (m, 4H) | 642.52 |
| 123 | 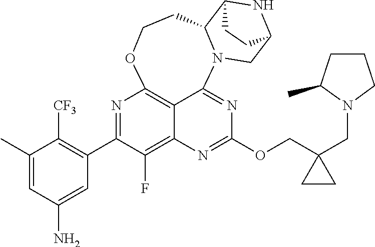 [SMILES: Cc1cc(N)cc(-c2nc3c4c(nc(OCC5(CN6CCC[C@@H]6C)CC5)nc4c2F)N2C[C@H]4CC[C@H](N4)[C@H]2CCO3)c1C(F)(F)F] | 3-((6aR,7S,10R)- 1-fluoro-13-((1- (((S)-2-methylpyrrolidin-1- yl)methyl)cyclopropyl) methoxy)-5,6,6a, 7,8,9,10,11-octahydro-4- oxa-3,11a,12,14,15- pentaaza-7,10- methanocyclohepta [4,5]cycloocta [1,2,3-de]naphthalen- 2-yl)-5-methyl-4- (trifluoromethyl)aniline | 6.69 (s, 1H), 6.46 (s, 1H), 4.53-4.18 (m, 6H), 4.06-3.60 (m, 6H), 3.06-2.70 (m, 2H), 2.43-2.42 (m, 3H), 2.19-1.93 (m, 9H), 1.68 (s, 1H), 1.35 (s, 3H), 0.88-0.73 (m, 4H) | 642.58 |

### Example 124 and 125: (1-((1-((((6aR,7S,10R)-2-(5-amino-3-methyl-2-(trifluoromethyl)phenyl)-1-fluoro-5,6,6a,7,8,9,10,11-octahydro-4-oxa-3,11a,12,14,15-pentaaza-7,10-methanocyclohepta[4,5]cycloocta[1,2,3-de]naphthalen-13-yl)oxy)methyl)cyclopropyl)methyl)pyrrolidin-2-yl)methanol

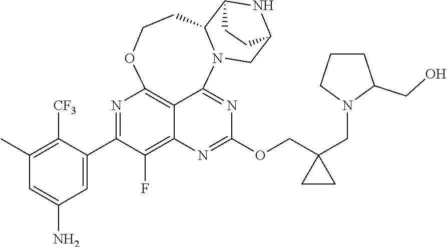 [SMILES: Cc1cc(N)cc(-c2nc3c4c(nc(OCC5(CN6CCCC6CO)CC5)nc4c2F)N2C[C@H]4CC[C@H](N4)[C@H]2CCO3)c1C(F)(F)F]

### Synthetic Route

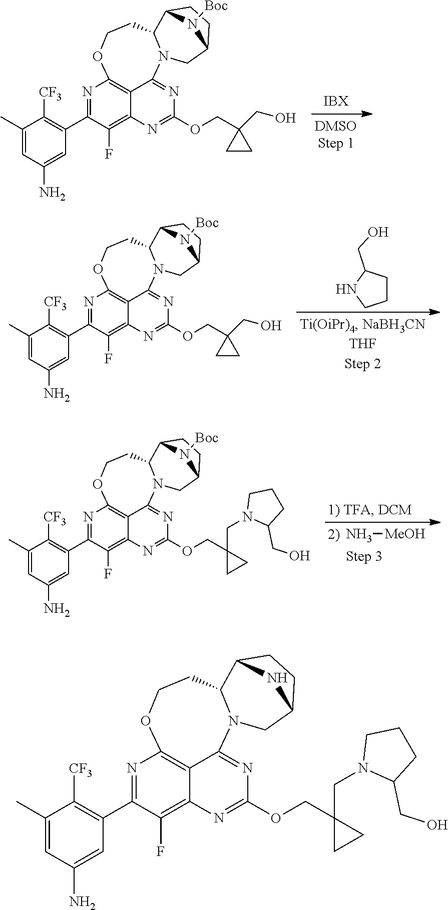

### Step 1: tert-butyl (6aR,7S,10R)-2-(5-amino-3-methyl-2-(trifluoromethyl)phenyl)-1-fluoro-13-((1-formylcyclopropyl)methoxy)-5,6,6a,7,8,9,10,11-octahydro-4-oxa-3,11a,12,14,15-pentaaza-7,10-methanocyclohepta[4,5]cycloocta[1,2,3-de]naphthalene-15-carboxylate

To a solution of tert-butyl (6aR,7S,10R)-2-(5-amino-3-methyl-2-(trifluoromethyl)phenyl)-1-fluoro-13-((1-(hydroxymethyl)cyclopropyl)methoxy)-5,6,6a,7,8,9,10,11-octahydro-4-oxa-3,11a,12,14,15-pentaaza-7,10-methanocyclohepta[4,5]cycloocta[1,2,3-de]naphthalene-15-carboxylate (220 mg, 0.33 mmol) in DMSO (9 mL) was added IBX (183 mg, 0.65 mmol). After stirring for 3 hours at 100° C., the reaction mixture was cooled to rt, diluted with brine, and extracted with EtOAc for 3 times. The combined organic phase was concentrated in vacuo. The resulting residue was purified by silica gel column chromatography to give the title product (88 mg). MS (ESI, m/e) [M+1]⁺ 673.4.

### Step 2: tert-butyl (6aR,7S,10R)-2-(5-amino-3-methyl-2-(trifluoromethyl)phenyl)-1-fluoro-13-((1-((2-(hydroxymethyl)pyrrolidin-1-yl)methyl)cyclopropyl)methoxy)-5,6,6a,7,8,9,10,11-octahydro-4-oxa-3,11a,12,14,15-pentaaza-7,10-methanocyclohepta[4,5]cycloocta[1,2,3-de]naphthalene-15-carboxylate

To a mixture of tert-butyl (6aR,7S,10R)-2-(5-amino-3-methyl-2-(trifluoromethyl)phenyl)-1-fluoro-13-((1-formylcyclopropyl)methoxy)-5,6,6a,7,8,9,10,11-octahydro-4-oxa-3,11a,12,14,15-pentaaza-7,10-methanocyclohepta[4,5]cycloocta[1,2,3-de]naphthalene-15-carboxylate (67 mg, 0.1 mmol) and pyrrolidin-2-ylmethanol (20 mg, 0.2 mmol) in THF (2 mL) was added Ti(OiPr)₄ (56 mg, 0.2 mmol). The mixture was stirred at room temperature for 30 minutes, followed by addition of NaBH₃CN (15 mg, 0.23 mmol). The resulting mixture was stirred at room temperature for 16 hours, which was then quenched with MeOH (5 mL). The mixture was concentrated in vacuo, and purified by silica gel column chromatography to give the title product (36 mg). MS (ESI, m/e) [M+1]⁺ 758.6.

### Step 3: (1-((1-((((6aR,7S,10R)-2-(5-amino-3-methyl-2-(trifluoromethyl)phenyl)-1-fluoro-5,6,6a,7,8,9,10,11-octahydro-4-oxa-3,11a,12,14,15-pentaaza-7,10-methanocyclohepta[4,5]cycloocta[1,2,3-de]naphthalen-13-yl)oxy)methyl)cyclopropyl)methyl)pyrrolidin-2-yl)methanol

To a solution of tert-butyl (6aR,7S,10R)-2-(5-amino-3-methyl-2-(trifluoromethyl)phenyl)-1-fluoro-13-((1-((2-(hydroxymethyl)pyrrolidin-1-yl)methyl)cyclopropyl)methoxy)-5,6,6a,7,8,9,10,11-octahydro-4-oxa-3,11a,12,14,15-pentaaza-7,10-methanocyclohepta[4,5]cycloocta[1,2,3-de]naphthalene-15-carboxylate (35 mg, 0.046 mmol) in DCM (2 mL) was added TFA (1 mL) at room temperature. After stirring at room temperature for 2 hours, the mixture was concentrated in vacuo and basified by NH₃/MeOH (7 M) to pH~9. The resulting mixture was purified by prep-TLC (DCM/MeOH(with NH₃)=8:1) to give Example 124 as the first eluting isomer (3 mg) and Example 125 as the second eluting isomer (6 mg).

Example 124: ¹H NMR (400 MHz, CD₃OD) δ 6.70 (s, 1H), 6.50-6.35 (m, 1H), 4.50-4.27 (m, 5H), 3.93-3.83 (m, 4H), 3.70-3.61 (m, 2H), 3.59-3.51 (m, 1H), 3.07-2.93 (m, 1H), 2.42 (s, 3H), 2.19-2.03 (m, 7H), 1.97-1.85 (m, 4H), 1.33-1.26 (m, 3H), 0.93-0.88 (m, 3H). MS (ESI, m/e) [M+1]⁺ 658.5.

Example 125: H NMR (400 MHz, CD₃OD) δ 6.68 (s, 1H), 6.43 (d, J=23.6 Hz, 1H), 4.80-4.63 (m, 1H), 4.49-4.18 (m, 3H), 4.17-3.81 (m, 3H), 3.68 (s, 1H), 3.63-3.39 (m, 5H), 2.84-2.60 (m, 1H), 2.42 (s, 3H), 2.13-1.62 (m, 11H), 1.28 (s, 1H), 0.81-0.58 (m, 3H), 0.53 (s, 1H). MS (ESI, m/e) [M+1]⁺ 658.5.

### Example 126 to 129: 3-((6aR,7S,10R)-1-fluoro-13-((5-methoxy-1,3-dimethylpiperidin-3-yl)methoxy)-5,6,6a,7,8,9,10,11-octahydro-4-oxa-3,11a,12,14,15-pentaaza-7,10-methanocyclohepta[4,5]cycloocta[1,2,3-de]naphthalen-2-yl)-5-methyl-4-(trifluoromethyl)aniline

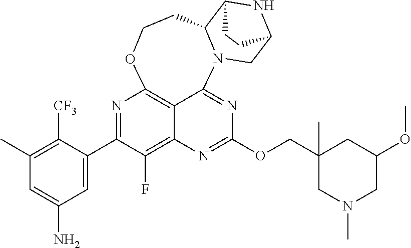

### Synthetic Route

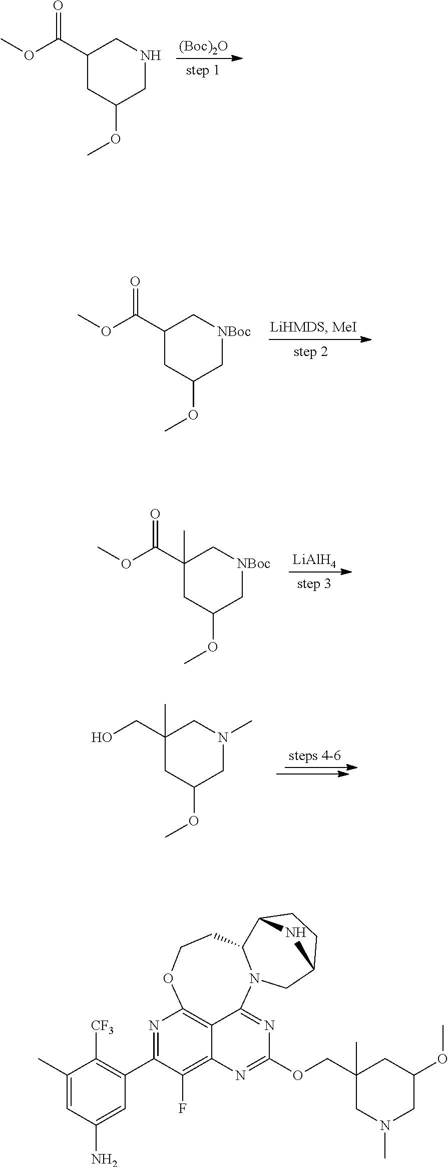

### Step 1: 1-(tert-butyl) 3-methyl 5-methoxypiperidine-1,3-dicarboxylate

To a solution of methyl 5-methoxypiperidine-3-carboxylate hydrogen chloride (0.95 g, 4.6 mmol) in DCM (100 mL) was added DIPEA (2.95 g, 22.7 mmol) and di-tert-butyl dicarbonate (1.49 g, 6.8 mmol). The resulting mixture was stirred at room temperature for 16 hours. The mixture was then diluted with brine (30 mL), and extracted with DCM (20 mL) for 3 times. The combined organic phase was dried over Na₂SO₄, filtered and concentrated in vacuo. The crude was purified by column chromatography (EtOAc in PE 30% to 100%) to give the title product (1.10 g). MS (ESI, m/e) [M+Na]⁺ 296.3.

### Step 2: 1-(tert-butyl) 3-methyl 5-methoxy-3-methylpiperidine-1,3-dicarboxylate

To a solution of 1-(tert-butyl) 3-methyl 5-methoxypiperidine-1,3-dicarboxylate (1.0 g, 3.6 mmol) in THF (50 mL) was added LiHMDS dropwise at 0° C. The resulting mixture was stirred at 0° C. for 20 min, followed by slow addition of Mel (1.0 g, 7.3 mmol). The reaction was quenched after 2 hours with saturated ammonium chloride solution (20 mL). The mixture was extracted with EtOAc (20 mL) for 3 times, washed with brine (10 mL), dried over Na₂SO₄ and concentrated in vacuo. The crude was purified by column chromatography (pure EtOAc) to give the title product (474 mg). MS (ESI, m/e) [M+Na]⁺ 310.2.

### Step 3: (5-methoxy-1,3-dimethylpiperidin-3-yl)methanol

To a solution of 1-(tert-butyl) 3-methyl 5-methoxy-3-methylpiperidine-1,3-dicarboxylate (0.37 g, 1.3 mmol) in THF (30 mL) was added LiAlH₄ (0.21 g, 5.2 mmol). The resulting mixture was stirred at 60° C. for 16 hours. The reaction mixture was then cooled to 0° C., dilute with ether (20 mL), and slowly added H₂O (0.2 mL), 15% aqueous sodium hydroxide (0.2 mL), and H₂O (0.6 mL) sequentially. The slurry was warmed to rt and stirred for 15 min, followed by addition of anhydrous magnesium sulfate. The mixture was further stirred for 15 min, and then filtered to removed salts. The filtrate was concentrated in vacuo to give the title product (260 mg), which was used directly in the next step without further purification. MS (ESI, m/e) [M+H]⁺ 174.1.

Steps 4-6: Examples 126-129 was prepared in similar fashion as Example 01 by replacing (2,2-difluorotetrahydro-1H-pyrrolizin-7a(5H)-yl)methanol with abovementioned (5-methoxy-1,3-dimethylpiperidin-3-yl)methanol. The compound was further separated via chiral Prep-HPLC (Column: CHIRALPAK® IE, 20 mm×250 mm, 5 μm; Mobile Phase A: n-Hexane, Mobile Phase B: EtOH (with 0.2% 2M NH₃ in MeOH); Flow Rate: 18 mL/min; Room Temperature; Gradient: 70% B in 18 min), followed by chiral Prep-HPLC (Column: i-Cellulose-5, 21.2 mm×250 mm, 5 μm; Mobile Phase A: n-Hexane, Mobile Phase B: EtOH (with 0.2% 2M NH₃ in MeOH); Flow Rate: 18 mL/min; Room Temperature; Gradient: 50% B in 18 min) to give 4 isomers as Examples 126-129:

Example 126: H NMR (500 MHz, CD₃OD) δ 6.68 (s, 1H), 6.44 (d, J=26.8 Hz, 1H), 4.33-4.08 (m, 7H), 3.69 (s, 1H), 3.61-3.48 (m, 2H), 3.34 (s, 3H), 3.02 (d, J=7.9 Hz, 1H), 2.80 (d, J=11.5 Hz, 1H), 2.42 (s, 3H), 2.27 (s, 3H), 2.18-1.68 (m, 9H), 1.11 (s, 3H), 1.08-0.99 (m, 1H). [M+H]⁺ 646.55.

Example 127: ¹H NMR (500 MHz, CD₃OD) δ 6.68 (s, 1H), 6.44 (d, J=31.2 Hz, 1H), 4.56-3.89 (m, 7H), 3.69 (s, 1H), 3.59 (s, 2H), 3.01 (d, J=7.5 Hz, 1H), 2.78 (d, J=11.3 Hz, 1H), 2.42 (s, 3H), 2.27 (s, 3H), 2.20-1.67 (m, 9H), 1.29 (s, 2H), 1.11 (s, 3H), 1.09-0.99 (m, 1H). [M+H]⁺ 646.55.

Example 128: ¹H NMR (500 MHz, CD₃OD) δ 6.68 (s, 1H), 6.44 (d, J=23.8 Hz, 1H), 4.61-3.90 (m, 7H), 3.70 (s, 1H), 3.56-2.54 (m, 2H), 3.34 (s, 3H), 2.72 (s, 1H), 2.55-2.34 (m, 5H), 2.29 (s, 3H), 2.21-1.76 (m, 7H), 1.67 (d, J=9.7 Hz, 1H), 1.56 (s, 1H), 1.29 (s, 2H), 1.16 (s, 3H). [M+H]⁺ 646.55.

Example 129: H NMR (500 MHz, CD₃OD) δ 6.69 (s, 1H), 6.45 (s, 1H), 4.54-3.97 (m, 7H), 3.74 (s, 1H), 3.56 (s, 2H), 3.35 (s, 4H), 2.42 (s, 4H), 2.32 (s, 3H), 1.79-1.59 (m, 9H), 1.16 (s, 3H), 1.14-1.03 (m, 1H). [M+H]⁺ 646.55.

Examples 130-132: The following examples were prepared in a manner similar to that in Example 07 by replacing (3R,4R)-3-aminotetrahydro-2H-pyran-4-ol with the corresponding alkyl alcohols.

| | | | | MS |
|---|---|---|---|---|
| | | | | (ESI, |
| Ex- | | | 1H NMR (500 | m/e) |
| ample | Structure | Compound Name | MHz, CD₃OD) δ | [M + H]⁺ |
| 130 | 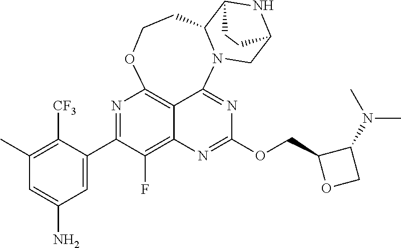 | (2R,3R)-2-((((6aR,7S,10R)-2-(5- amino-3-methyl-2- (trifluoromethyl)phenyl)-1-fluoro- 5,6,6a,7,8,9,10,11-octahydro-4- oxa-3,11a,12,14,15-pentaaza-7,10- methanocyclohepta[4,5]cycloocta [1,2,3-de]naphthalen-13- yl)oxy)methyl)-N,N- dimethyloxetan-3-amine | 6.69 (s, 1H), 6.46- 6.40 (m,1H), 5.17- 5.16 (m, 1H), 4.63- 3.44 (m, 12H), 2.42 (s, 3H), 2.21 (s, 6H), 2.10-1.87 (m, 6H). | 604.5 |
| 131 | 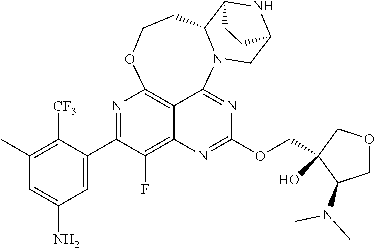 [SMILES: Cc1cc(N)cc(-c2nc3c4c(nc(OC[C@]5(O)COC[C@H]5N(C)C)nc4c2F)N2C[C@H]4CC[C@H](N4)[C@H]2CCO3)c1C(F)(F)F] | (3R,4R)-3-((((6aR,7S,10R)-2-(5- amino-3-methyl-2- (trifluoromethyl)phenyl)-1-fluoro- 5,6,6a,7,8,9,10,11-octahydro-4- oxa-3,11a,12,14,15-pentaaza-7,10- methanocyclohepta[4,5]cycloocta [1,2,3-de]naphthalen-13- yl)oxy)methyl)-4- (dimethylamino)tetrahydrofuran-3- ol | 6.70 (s, 1H), 6.46- 6.42 (m, 1H), 4.62- 4.50 (m, 2H), 4.37- 4.28 (m, 2H), 4.20-3.98 (m, 5H), 3.86-3.69 (m, 3H), 3.53-3.39 (m, 2H), 2.72 (m, 6H), 2.43-2.42 (m, 3H), 2.23-1.79 (m, 6H). | 634.5 |
| 132 | 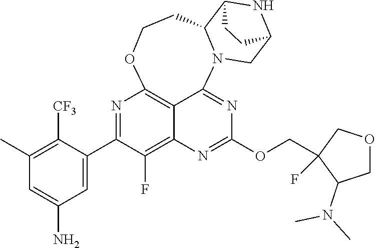 [SMILES: Cc1cc(N)cc(-c2nc3c4c(nc(OCC5(F)COCC5N(C)C)nc4c2F)N2C[C@H]4CC[C@H](N4)[C@H]2CCO3)c1C(F)(F)F] | 4-((((6aR,7S,10R)-2-(5-amino-3- methyl-2-(trifluoromethyl)phenyl)- 1-fluoro-5,6,6a,7,8,9,10,11- octahydro-4-oxa-3,11a,12,14,15- pentaaza-7,10- methanocyclohepta[4,5]cycloocta [1,2,3-de]naphthalen-13- yl)oxy)methyl)-4-fluoro-N,N- dimethyltetrahydrofuran-3-amine | 6.69 (s, 1H), 6.46- 6.41 (m, 1H), 4.96- 4.90 (m, 1H), 4.62- 3.55 (m, 13H), 3.18- 3.10 (m, 1H), 2.44- 2.42 (m, 9H), 2.09- 1.82 (m, 6H). | 636.5 |
| | (mixture of two isomers) | | | |

Examples 133-134: The following examples were prepared by further separation of Example 63 via chiral Prep-HPLC (Column: i-Cellulose-5, 21.2 mm×250 mm, 5 μm; Mobile Phase A: n-Hexane, Mobile Phase B: EtOH (with 0.2% 2M NH₃ in MeOH); Flow Rate: 18 mL/min; Temperature: 25° C.; Gradient: 50% B in 18 min).

| | | | | MS |
|---|---|---|---|---|
| | | | | (ESI, |
| Ex- | | | 1H NMR (500 | m/e) |
| ample | Structure | Compound Name | MHz, DMSO-d8) δ | [M + H]⁺ |
| 133 | 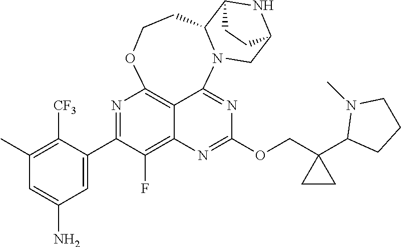 [SMILES: Cc1cc(N)cc(-c2nc3c4c(nc(OCC5(C6CCCN6C)CC5)nc4c2F)N2C[C@H]4CC[C@H](N4)[C@H]2CCO3)c1C(F)(F)F] | 3-((6aR,7S,10R)-1-fluoro-13-((1-(1- methylpyrrolidin-2- yl)cyclopropyl)methoxy)- 5,6,6a,7,8,9,10,11-octahydro-4-oxa- 3,11a,12,14,15-pentaaza-7,10- methanocyclohepta[4,5]cycloocta [1,2,3-de]naphthalen-2-yl)-5-methyl- 4-(trifluoromethyl)aniline | 6.59 (s, 1H), 6.36- 6.35 (m, 1H), 5.86 (s, 2H), 4.54-3.44 (m, 10H), 2.49- 2.27 (m, 9H), 2.06- 1.45 (m, 10H), 0.67-0.38 (m, 4H). | 628.5 |
| | (isomer 1) | | | |
| 134 | 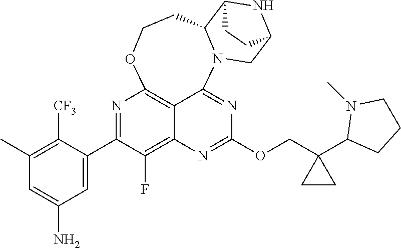 [SMILES: Cc1cc(N)cc(-c2nc3c4c(nc(OCC5(C6CCCN6C)CC5)nc4c2F)N2C[C@H]4CC[C@H](N4)[C@H]2CCO3)c1C(F)(F)F] | 3-((6aR,7S,10R)-1-fluoro-13-((1-(1- methylpyrrolidin-2- yl)cyclopropyl)methoxy)- 5,6,6a,7,8,9,10,11-octahydro-4-oxa- 3,11a,12,14,15-pentaaza-7,10- methanocyclohepta[4,5]cycloocta [1,2,3-de]naphthalen-2-yl)-5-methyl- 4-(trifluoromethyl)aniline | 6.54 (s, 1H), 6.30 (s, 1H), 5.66 (s, 2H), 4.54-3.44 (m, 10H), 2.49-2.27 (m, 9H), 2.06-1.40 (m, 10H), 0.67- 0.31 (m, 4H). | 628.5 |
| | (isomer 2) | | | |

Examples 135: The following example was prepared in a manner similar to that in Examples 59-62 by replacing (2,2-difluorocyclopropane-1,1-diyl)dimethanol with cyclopropane-1,1-diyldimethanol.

| | | | | MS |
|---|---|---|---|---|
| | | | 1H NMR | (ESI, |
| Ex- | | | (500 MHz, | m/e) |
| ample | Structure | Compound Name | CD₃OD) δ | [M + H]⁺ |
| 135 | 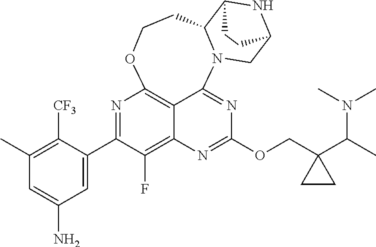 | 3-((6aR,7S,10R)-13-((1-(1- (dimethylamino)ethyl)cyclopropyl) methoxy)-1-fluoro- 5,6,6a,7,8,9,10,11-octahydro-4-oxa- 3,11a,12,14,15-pentaaza-7,10- methanocyclohepta[4,5]cycloocta [1,2,3-de]naphthalen-2-yl)-5-methyl- 4-(trifluoromethyl)aniline | 6.69 (s, 1H), 6.46- 6.40 (m, 1H), 4.51- 3.45 (m, 9H), 2.56- 2.42 (m, 10H), 2.08- 1.62 (m, 6H), 1.12- 1.11 (m, 3H), 0.91- 0.69 (m, 4H). | 616.5 |
| | (mixture of two isomers) | | | |

Examples 136-137: The following examples were prepared by further separation of Example 49 as a mixture of two isomers via chiral Prep-HPLC (Column: CHIRAL ART Amylose-SA, 2×25 cm, 5 μm; Mobile Phase A: n-Hexane, Mobile Phase B: EtOH (with 1% 2M NH₃ in MeOH); Flow Rate: 20 mL/min; Temperature: 25° C.; Gradient: Isocratic 40%).

| | | | | MS |
|---|---|---|---|---|
| | | | | (ESI, |
| Ex- | | | 1H NMR (500 MHz, | m/e) |
| ample | Structure | Compound Name | CD₃OD) δ | [M + H]⁺ |
| 136 | 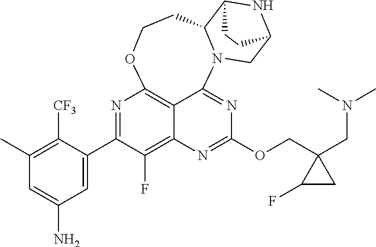 | 3-((6aR,7S,10R)-13-((1- ((dimethylamino)methyl)-2- fluorocyclopropyl)methoxy)-1- fluoro-5,6,6a,7,8,9,10,11- octahydro-4-oxa-3,11a,12,14,15- pentaaza-7,10-methano- cyclohepta[4,5]cycloocta [1,2,3-de]naphthalen-2-yl)-5- methyl-4-(trifluoromethyl) aniline | 6.68 (s, 1H), 6.46-6.40 (m, 1H), 4.82-4.68 (m, 1H), 4.42-3.98 (m, 7H), 3.66-3.54 (m, 2H), 2.82-2.58 (m, 4H), 2.42 (s, 3H), 2.18-1.78 (m, 6H), 1.12-0.88 (m, 2H). | 620.5 |
| | (isomer 1) | | | |
| 137 | 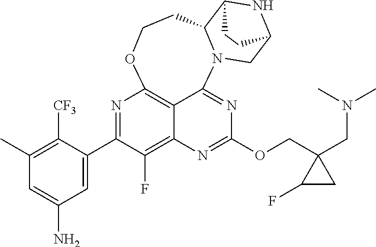 | 3-((6aR,7S,10R)-13-((1- ((dimethylamino)methyl)-2- fluorocyclopropyl)methoxy)-1- fluoro-5,6,6a,7,8,9,10,11- octahydro-4-oxa-3,11a,12,14,15- pentaaza-7,10-methano- cyclohepta[4,5]cycloocta [1,2,3-de]naphthalen-2-yl)-5- methyl-4-(trifluoromethyl) aniline | 6.68 (s, 1H), 6.46-6.40 (m, 1H), 4.83-4.69 (m, 1H), 4.42-3.88 (m, 7H), 3.66-3.53 (m, 2H), 2.84-2.54 (m, 4H), 2.42 (s, 3H), 2.17-1.78 (m, 6H), 1.13-0.89 (m, 2H). | 620.5 |
| | (isomer 2) | | | |

Examples 138-139: The following examples were prepared by further separation of Example 90 as a mixture of two isomers via chiral Prep-HPLC (Column: i-Cellulose-5, 21.2 mm×250 mm, 5 μM; Mobile Phase A: n-Hexane, Mobile Phase B: EtOH (with 0.2% 2M NH₃ in MeOH); Flow Rate: 18 mL/min; Temperature: 25° C.; Gradient: 50% B in 18 min).

| | | | | MS |
|---|---|---|---|---|
| | | | 1H NMR | (ESI, |
| Ex- | | | (500 MHz, | m/e) |
| ample | Structure | Compound Name | DMSO-d8) δ | [M + H]⁺ |
| 138 | 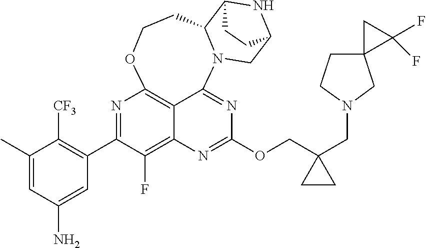 [SMILES: Cc1cc(N)cc(-c2nc3c4c(nc(OCC5(CN6CCC7(C6)CC7(F)F)CC5)nc4c2F)N2C[C@H]4CC[C@H](N4)[C@H]2CCO3)c1C(F)(F)F] | 3-((6aR,7S,10R)-13-((1- ((1,1-difluoro-5-azaspiro [2.4]heptan-5- yl)methyl)cyclopropyl) methoxy)-1-fluoro- 5,6,6a,7,8,9,10,11-octahydro- 4-oxa-3,11a,12,14,15- pentaaza[7,10] methanocyclohepta [4,5]cycloocta[1,2,3-de] naphthalen-2-yl)-5-methyl-4- (trifluoromethyl)aniline | 6.68 (s, 1H), 6.46- 6.45 (m,1H), 4.48- 3.57 (m, 9H), 2.83- 2.55 (m, 6H), 2.42 (s, 3H), 2.08-1.72 (m, 8H), 1.34-1.23 (m, 2H), 0.70-0.52 (m, 4H). | 690.5 |
| | (isomer 1) | | | |
| 139 | 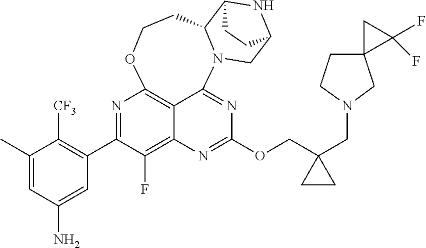 [SMILES: Cc1cc(N)cc(-c2nc3c4c(nc(OCC5(CN6CCC7(C6)CC7(F)F)CC5)nc4c2F)N2C[C@H]4CC[C@H](N4)[C@H]2CCO3)c1C(F)(F)F] | 3-((6aR,7S,10R)-13-((1- ((1,1-difluoro-5-azaspiro [2.4]heptan-5- yl)methyl)cyclopropyl) methoxy)-1-fluoro- 5,6,6a,7,8,9,10,11-octahydro- 4-oxa-3,11a,12,14,15- pentaaza[7,10] methanocyclohepta [4,5]cycloocta[1,2,3-de] naphthalen-2-yl)-5-methyl-4- (trifluoromethyl)aniline | 6.68 (s, 1H), 6.46- 6.41 (m, 1H), 4.47- 3.63 (m, 9H), 2.72- 2.55 (m, 6H), 2.42 (s, 3H), 2.08-1.73 (m, 8H), 1.34-1.26 (m, 2H), 0.70-0.52 (m, 4H). | 690.5 |
| | (isomer 2) | | | |

Examples 140-143: The following examples were prepared in a manner similar to that in Example 37 by by replacing the corresponding substitution or coupling partners.

| | | | | MS |
|---|---|---|---|---|
| | | | | (ESI, |
| Ex- | | | 1H NMR (500 MHz, | m/e) |
| ample | Structure | Compound Name | CD₃OD) δ | [M + H]⁺ |
| 140 | 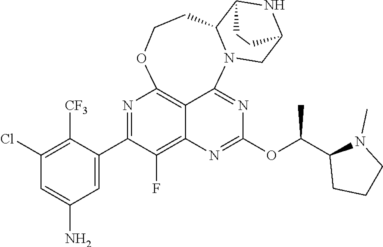 [SMILES: C[C@H](Oc1nc2c3c(nc(-c4cc(N)cc(Cl)c4C(F)(F)F)c(F)c3n1)OCC[C@@H]1[C@@H]3CC[C@H](CN21)N3)[C@@H]1CCCN1C] | 3-chloro-5-((6aR,7S,10R)-1- fluoro-13-((S)-1-((S)-1- methylpyrrolidin-2-yl)ethoxy)- 5,6,6a,7,8,9,10,11-octahydro-4- oxa-3,11a,12,14,15-pentaaza- 7,10-methano- cyclohepta[4,5]cycloocta [1,2,3-de]naphthalen-2-yl)-4- (trifluoromethyl)aniline | 6.88 (d, J = 2.0 Hz, 1H), 6.51 (d, J = 37.5 Hz, 1H), 5.30-5.19 (m, 1H), 4.42-4.23 (m, 3H), 4.13-3.87 (m, 2H), 3.67 (s, 1H), 3.63-3.51 (m, 1H), 3.09 (s, 1H), 2.76 (s, 1H), 2.55 (s, 3H), 2.41 (s, 1H), 2.17- 1.94 (m, 4H), 1.92- 1.69 (m, 6H), 1.40 (d, J = 6.1 Hz, 3H). | 622.5 |
| 141 | 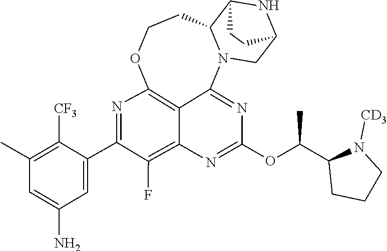 [SMILES: [2H]C([2H])([2H])N1CCC[C@H]1[C@H](C)Oc1nc2c3c(nc(-c4cc(N)cc(C)c4C(F)(F)F)c(F)c3n1)OCC[C@@H]1[C@@H]3CC[C@H](CN21)N3] | 3-((6aR,7S,10R)-1-fluoro-13- ((S)-1-((S)-1-(methyl- d3)pyrrolidin-2-yl)ethoxy)- 5,6,6a,7,8,9,10,11-octahydro-4- oxa-3,11a,12,14,15-pentaaza- 7,10-methano- cyclohepta[4,5]cycloocta [1,2,3-de]naphthalen-2-yl)-5- methyl-4- (trifluoromethyl)aniline | 6.69 (s, 1H), 6.43 (d, J = 30.7 Hz, 1H), 5.29- 5.25 (m, 1H), 4.42-4.22 (m, 3H), 4.15-3.90 (m, 2H), 3.75-3.55 (m, 3H), 3.35 (s, 1H), 3.10-2.55 (m, 1H), 2.42 (d, J = 1.8 Hz, 3H), 2.23- 1.71 (m, 10H), 1.43 (d, J = 6.0 Hz, 3H). (1 H obscured by solvent peak) | 605.6 |
| 142 | 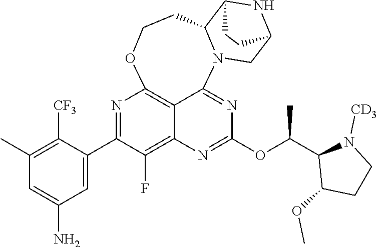 [SMILES: [2H]C([2H])([2H])N1CC[C@H](OC)[C@H]1[C@H](C)Oc1nc2c3c(nc(-c4cc(N)cc(C)c4C(F)(F)F)c(F)c3n1)OCC[C@@H]1[C@@H]3CC[C@H](CN21)N3] | 3-((6aR,7S,10R)-1-fluoro-13- ((S)-1-((2R,3S)-3-methoxy-1- (methyl-d3)pyrrolidin-2- yl)ethoxy)-5,6,6a,7,8,9,10,11- octahydro-4-oxa- 3,11a,12,14,15- pentaaza-7,10-methano- cyclohepta[4,5]cycloocta [1,2,3-de]naphthalen-2-yl)-5- methyl-4- (trifluoromethyl)aniline | 6.68 (s, 1H), 6.44 (d, J = 27.7 Hz, 1H), 5.32- 5.28 (m, 1H), 4.44-4.20 (m, 3H), 4.13-3.90 (m, 2H), 3.85 (d, J = 5.3 Hz, 1H), 3.67 (s, 1H), 3.61-3.52(m, 1H), 3.28 (s, 3H), 2.99 (t, J = 7.6 Hz, 1H), 2.77-2.65 (m, 1H), 2.58-2.49 (m, 1H), 2.42 (d, J = 1.9 Hz, 3H), 2.18-1.70 (m, 8H), 1.43 (d, J = 6.3 Hz, 3H). | 635.6 |
| 143 | 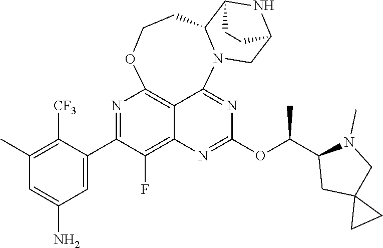 [SMILES: Cc1cc(N)cc(-c2nc3c4c(nc(O[C@@H](C)[C@@H]5CC6(CC6)CN5C)nc4c2F)N2C[C@H]4CC[C@H](N4)[C@H]2CCO3)c1C(F)(F)F] | 3-((6aR,7S,10R)-1-fluoro-13- ((S)-1-((S)-5-methyl-5- azaspiro[2.4]heptan-6- yl)ethoxy)-5,6,6a,7,8,9,10,11- octahydro-4-oxa- 3,11a,12,14,15-pentaaza[7,10] methanocyclohepta [4,5]cycloocta[1,2,3- de]naphthalen-2-yl)- 5-methyl-4- (trifluoromethyl)aniline | 6.68 (s, 1H), 6.47-6.41 (m, 1H), 5.37-5.32 (m, 1H), 4.36-3.92 (m, 5H), 3.66-3.55 (m, 2H), 3.01-3.00 (m, 1H), 2.74-2.63 (m, 2H), 2.55 (s, 3H), 2.42 (s, 3H), 2.18-1.65 (m, 8H), 1.40 (d, J = 5 Hz, 3H), 0.64- 0.55 (m, 4H). | 628.5 |

Examples 144-145: The following examples were prepared in a manner similar to that in Example 75-76.

| | | | | MS |
|---|---|---|---|---|
| | | | | (ESI, |
| Ex- | | | 1H NMR (500 MHz, | m/e) |
| ample | Structure | Compound Name | CD₃OD) δ | [M + H]⁺ |
| 144 | 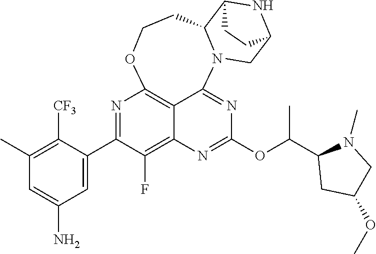 [SMILES: CO[C@@H]1C[C@@H](C(C)Oc2nc3c4c(nc(-c5cc(N)cc(C)c5C(F)(F)F)c(F)c4n2)OCC[C@@H]2[C@@H]4CC[C@H](CN32)N4)N(C)C1] | 3-((6aR,7S,10R)-1-fluoro-13-(1- ((2S,4R)-4-methoxy-1- methylpyrrolidin-2-yl)ethoxy)- 5,6,6a,7,8,9,10,11-octahydro-4- oxa-3,11a,12,14,15-pentaaza- 7,10-methano- cyclohepta[4,5]cycloocta [1,2,3-de]naphthalen-2-yl)-5- methyl-4- (trifluoromethyl)aniline | 6.68 (s, 1H), 6.46-6.41 (m, 1H), 5.32-5.26 (m, 1H), 4.35-4.28 (m, 3H), 4.07-3.93 (m, 3H), 3.69-3.57 (m, 2H), 3.38-3.31 (m, 3H), 3.05-2.97 (m, 1H), 2.57-2.42 (m,7H), 2.09- 1.88 (m, 8H), 1.39-1.12 (m, 4H) | 632.5 |
| | (isomer 1) | | | |
| 145 | 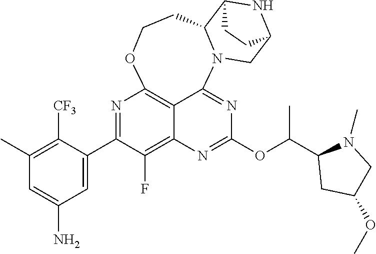 [SMILES: CO[C@@H]1C[C@@H](C(C)Oc2nc3c4c(nc(-c5cc(N)cc(C)c5C(F)(F)F)c(F)c4n2)OCC[C@@H]2[C@@H]4CC[C@H](CN32)N4)N(C)C1] | 3-((6aR,7S,10R)-1-fluoro-13-(1- ((2S,4R)-4-methoxy-1- methylpyrrolidin-2-yl)ethoxy)- 5,6,6a,7,8,9,10,11-octahydro-4- oxa-3,11a,12,14,15-pentaaza- 7,10-methano- cyclohepta[4,5]cycloocta [1,2,3-de]naphthalen-2-yl)-5- methyl-4- (trifluoromethyl)aniline | 6.68 (s, 1H), 6.46-6.41 (m, 1H), 5.32-5.26 (m, 1H), 4.35-4.28 (m, 3H), 4.07-3.93 (m, 3H), 3.70-3.57 (m, 2H), 3.32 (s, 3H), 2.86-2.77 (m, 1H), 2.47-2.42 (m, 7H), 2.12-1.84 (m, 8H), 1.38-1.12 (m, 4H). | 632.5 |
| | (isomer 2) | | | |

Examples 146-153: The following examples were prepared in a manner similar to that in Example 77 by replacing the corresponding substitution or coupling partners. The products were purified by prep-TLC (DCM/MeOH(with NH₃)=8:1).

| | | | | MS |
|---|---|---|---|---|
| Ex- | | | | (ESI, |
| am- | | | 1H NMR (500 MHz, | m/e) |
| ple | Structure | Compound Name | CD₃OD) δ | [M + H]⁺ |
| 146 | 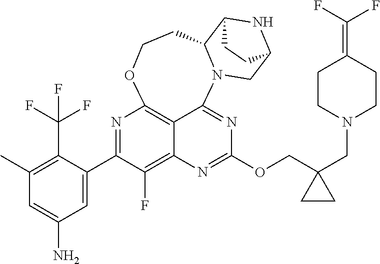 [SMILES: Cc1cc(N)cc(-c2nc3c4c(nc(OCC5(CN6CCC(=C(F)F)CC6)CC5)nc4c2F)N2C[C@H]4CC[C@H](N4)[C@H]2CCO3)c1C(F)(F)F] | 3-((6aR,7S,10R)-13-((1-((4- (difluoromethylene) piperidin-1-yl)methyl) cyclopropyl)methoxy)- 1-fluoro-5,6,6a,7,8,9,10,11- octahydro-4-oxa- 3,11a,12,14,15- pentaaza-7,10-methano- cyclohepta[4,5]cycloocta [1,2,3-de]naphthalen-2-yl)-5- methyl-4-(trifluoromethyl) aniline | 6.66 (s, 1H), 6.41 (d, J = 23.2 Hz, 1H), 4.54- 4.20 (m, 5H), 4.16- 3.92 (m, 2H), 3.77 (s, 1H), 3.72-3.58 (m, 1H), 2.55-2.48 (m, 4H), 2.48-2.41 (m, 2H), 2.41-2.37 (m, 3H), 2.25-2.01 (m, 7H), 1.94-1.78 (m, 3H), 0.76-0.66 (m, 2H), 0.53-0.45 (m, 2H). | 690.4 |
| 147 | 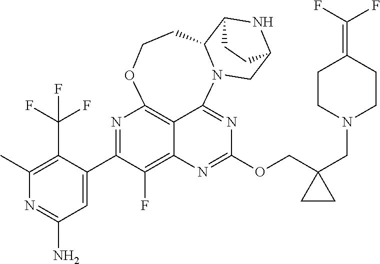 [SMILES: Cc1nc(N)cc(-c2nc3c4c(nc(OCC5(CN6CCC(=C(F)F)CC6)CC5)nc4c2F)N2C[C@H]4CC[C@H](N4)[C@H]2CCO3)c1C(F)(F)F] | 4-((6aR,7S,10R)-13-((1-((4- (difluoromethylene) piperidin-1- yl)methyl)cyclopropyl) methoxy)-1-fluoro- 5,6,6a,7,8,9,10,11- octahydro-4-oxa- 3,11a,12,14,15-pentaaza- 7,10-methano- cyclohepta[4,5]cycloocta [1,2,3-de]naphthalen-2-yl)-6- methyl-5- (trifluoromethyl)pyridin- 2-amine | 6.34 (s, 1H), 4.50- 4.31 (m, 3H), 4.31- 3.93 (m, 3H), 3.80- 3.69 (m, 1H), 3.70- 3.56 (m, 1H), 3.28- 3.17 (m, 3H), 2.60- 2.38 (m, 8H), 2.20- 2.00 (m, 6H), 1.99- 1.73 (m, 3H), 0.77- 0.60 (m, 2H), 0.57- 0.36 (m, 2H). | 691.5 |
| 148 | 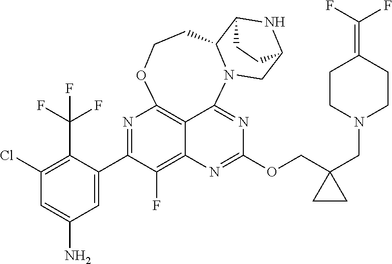 [SMILES: Nc1cc(Cl)c(C(F)(F)F)c(-c2nc3c4c(nc(OCC5(CN6CCC(=C(F)F)CC6)CC5)nc4c2F)N2C[C@H]4CC[C@H](N4)[C@H]2CCO3)c1] | 3-chloro-5-((6aR,7S,10R)- 13-((1-((4- (difluoromethylene) piperidin-1- yl)methyl)cyclopropyl) methoxy)- 1-fluoro-5,6,6a,7,8,9,10,11- octahydro-4-oxa- 3,11a,12,14,15- pentaaza-7,10-methano- cyclohepta[4,5]cycloocta [1,2,3-de]naphthalen-2-yl)-4- (trifluoromethyl)aniline | 6.86 (s, 1H), 6.49 (d, J = 21.6 Hz, 1H), 4.50- 4.22 (m, 4H), 4.20- 3.85 (m, 2H), 3.84- 3.63 (m, 2H), 2.63- 2.35 (m, 6H), 2.26- 2.06 (m, 6H), 2.05- 1.50 (m, 5H), 0.79- 0.66 (m, 2H), 0.56- 0.43 (m, 2H). | 710.4 |
| 149 | 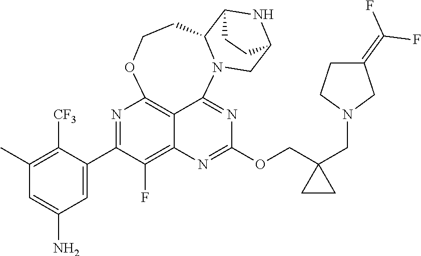 [SMILES: Cc1cc(N)cc(-c2nc3c4c(nc(OCC5(CN6CCC(=C(F)F)C6)CC5)nc4c2F)N2C[C@H]4CC[C@H](N4)[C@H]2CCO3)c1C(F)(F)F] | 3-((6aR,7S,10R)-13-((1-((3- (difluoromethylene) pyrrolidin-1-yl)methyl) cyclopropyl)methoxy)- 1-fluoro-5,6,6a,7,8,9,10,11- octahydro-4-oxa- 3,11a,12,14,15- pentaaza-7,10-methano- cyclohepta[4,5]cycloocta [1,2,3-de]naphthalen-2-yl)-5- methyl-4-(trifluoromethyl) aniline | 6.65 (s, 1H), 6.40 (d, J = 25.2 Hz, 1H), 4.47- 4.14 (m, 5H), 4.13- 3.85 (m, 2H), 3.75- 3.46 (m, 2H), 3.22- 3.09 (m, 2H), 2.76- 2.63 (m, 2H), 2.62- 2.44 (m, 2H), 2.44- 2.30 (m, 5H), 2.12- 1.73 (m, 6H), 0.76- 0.58 (m, 2H), 0.58- 0.39 (m, 2H). | 676.4 |
| 150 | 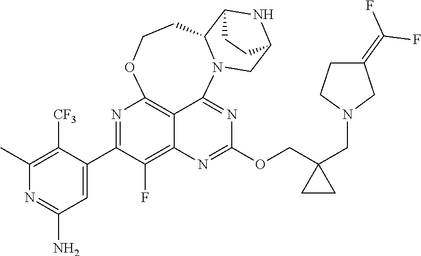 [SMILES: Cc1nc(N)cc(-c2nc3c4c(nc(OCC5(CN6CCC(=C(F)F)C6)CC5)nc4c2F)N2C[C@H]4CC[C@H](N4)[C@H]2CCO3)c1C(F)(F)F] | 4-((6aR,7S,10R)-13-((1-((3- (difluoromethylene) pyrrolidin-1-yl)methyl) cyclopropyl)methoxy)- 1-fluoro-5,6,6a,7,8,9,10,11- octahydro-4-oxa- 3,11a,12,14,15- pentaaza-7,10-methano- cyclohepta[4,5]cycloocta [1,2,3-de]naphthalen-2-yl)-6- methyl-5- (trifluoromethyl)pyridin- 2-amine | 6.35 (s, 1H), 4.49- 4.33 (m, 3H), 4.32- 3.88 (m, 4H), 3.87- 3.75 (m, 1H), 3.74- 3.65 (m, 1H), 3.26- 3.12 (m, 3H), 2.76- 2.64 (m, 2H), 2.62- 2.47 (m, 5H), 2.43- 2.27 (m, 2H), 2.14- 2.03 (m, 2H), 1.98- 1.80 (m, 3H), 0.74- 0.66 (m, 2H), 0.57- 0.48 (m, 2H). | 677.5 |
| 151 | 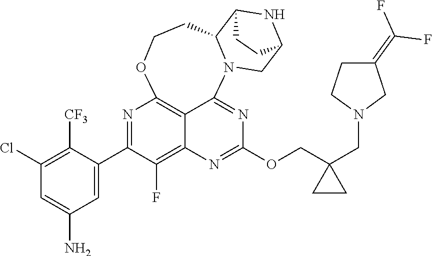 [SMILES: Nc1cc(Cl)c(C(F)(F)F)c(-c2nc3c4c(nc(OCC5(CN6CCC(=C(F)F)C6)CC5)nc4c2F)N2C[C@H]4CC[C@H](N4)[C@H]2CCO3)c1] | 3-chloro-5-((6aR,7S,10R)- 13-((1-((3- (difluoromethylene) pyrrolidin-1-yl)methyl) cyclopropyl)methoxy)- 1-fluoro-5,6,6a,7,8,9,10,11- octahydro-4-oxa- 3,11a,12,14,15- pentaaza-7,10-methano- cyclohepta[4,5]cycloocta [1,2,3-de]naphthalen-2-yl)-4- (trifluoromethyl)aniline | 6.87 (s, 1H), 6.49 (s, 1H), 4.51-4.24 (m, 4H), 4.11-3.82 (m, 3H), 3.27-3.22 (m, 3H), 2.85-2.70 (m, 2H), 2.70-2.48 (m, 2H), 2.48-2.25 (m, 3H), 2.25-1.84 (m, 6H), 0.77-0.67 (m, 2H), 0.60-0.49 (m, 2H). | 696.4 |
| 152 | 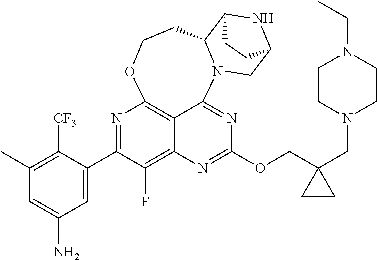 [SMILES: CCN1CCN(CC2(COc3nc4c5c(nc(-c6cc(N)cc(C)c6C(F)(F)F)c(F)c5n3)OCC[C@@H]3[C@@H]5CC[C@H](CN43)N5)CC2)CC1] | 3-((6aR,7S,10R)-13-((1-((4- ethylpiperazin-1-yl)methyl) cyclopropyl)methoxy)- 1-fluoro-5,6,6a,7,8,9,10,11- octahydro-4-oxa- 3,11a,12,14,15- pentaaza-7,10-methano- cyclohepta[4,5]cycloocta [1,2,3-de]naphthalen-2-yl)-5- methyl-4-(trifluoromethyl) aniline | 6.58 (s, 1H), 6.46 (s, 1H), 4.42-4.26 (m, 4H), 4.08-3.82 (m, 3H), 3.68 (s, 2H), 3.46 (s, 1H), 2.84-2.31 (m, 13H), 2.50-1.82 (m, 5H), 1.35-1.01 (m, 5H), 0.65 (s, 2H), 0.43 (s, 2H). | 671.5 |
| 153 | 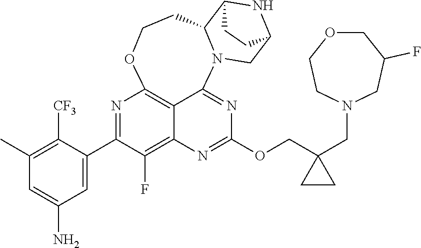 [SMILES: Cc1cc(N)cc(-c2nc3c4c(nc(OCC5(CN6CCOCC(F)C6)CC5)nc4c2F)N2C[C@H]4CC[C@H](N4)[C@H]2CCO3)c1C(F)(F)F] | 3-((6aR,7S,10R)-1- fluoro-13-((1-((6-fluoro- 1,4-oxazepan-4-yl)methyl) cyclopropyl)methoxy)- 5,6,6a,7,8,9,10,11- octahydro-4-oxa- 3,11a,12,14,15-pentaaza- 7,10-methano- cyclohepta[4,5]cycloocta [1,2,3-de]naphthalen-2-yl)-5- methyl-4-(trifluoromethyl) aniline | 6.59 (s, 1H), 6.46 (s, 1H), 4.78 (s, 1H), 4.65 (s, 1H), 4.47 (s, 1H), 4.31 (s, 3H), 3.90 (s, 3H), 3.86-3.77 (m, 3H), 3.65-3.51 (m, 2H), 3.16- 2.92 (m, 2H), 2.77 (s, 2H), 2.72-2.56 (m, 2H), 2.43 (d, J = 1.8 Hz, 4H), 2.05-1.84 (m, 4H), 1.23 (s, 2H), 0.67 (s, 2H), 0.46 (d, J = 4.7 Hz, 2H). | 676.5 |

Examples 154-156: The following examples were prepared in a manner similar to that in Example 110 by replacing the corresponding substitution or coupling partners. The products were purified by prep-TLC (DCM/MeOH(with NH₃)=8:1).

| | | | | MS |
|---|---|---|---|---|
| Ex- | | | 1H NMR | (ESI, |
| am- | | | (500 MHz, | m/e) |
| ple | Structure | Compound Name | CD₃OD) δ | [M + H]⁺ |
| 154 | 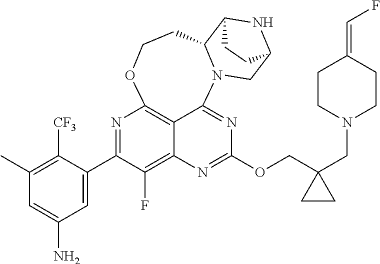 [SMILES: Cc1cc(N)cc(-c2nc3c4c(nc(OCC5(CN6CCC(=CF)CC6)CC5)nc4c2F)N2C[C@H]4CC[C@H](N4)[C@H]2CCO3)c1C(F)(F)F] | 3-((6aR,7S,10R)-1-fluoro-13-((1- ((4-(fluoromethylene)piperidin-1- yl)methyl)cyclopropyl)methoxy)- 5,6,6a,7,8,9,10,11-octahydro-4- oxa-3,11a,12,14,15-pentaaza- 7,10- methanocyclohepta[4,5]cycloocta [1,2,3-de]naphthalen-2-yl)-5- methyl-4-(trifluoromethyl)aniline | 6.66 (s, 1H), 6.62- 6.28 (m, 2H), 4.56- 4.16 (m, 5H), 4.15- 3.88 (m, 2H), 3.68 (s, 1H), 3.61-3.47 (m, 1H), 2.65-2.45 (m, 5H), 2.45-2.32 (m, 5H), 2.33-2.21 (m, 2H), 2.14-1.76 (m, 7H), 0.76-0.62 (m, 2H), 0.57-0.39 (m, 2H). | 672.5 |
| 155 | 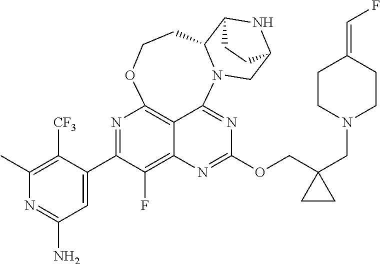 [SMILES: Cc1nc(N)cc(-c2nc3c4c(nc(OCC5(CN6CCC(=CF)CC6)CC5)nc4c2F)N2C[C@H]4CC[C@H](N4)[C@H]2CCO3)c1C(F)(F)F] | 4-((6aR,7S,10R)-1-fluoro-13-((1- ((4-(fluoromethylene)piperidin-1- yl)methyl)cyclopropyl)methoxy)- 5,6,6a,7,8,9,10,11-octahydro-4- oxa-3,11a,12,14,15-pentaaza- 7,10- methanocyclohepta[4,5]cycloocta [1,2,3-de]naphthalen-2-yl)-6- methyl-5- (trifluoromethyl)pyridin-2-amine | 6.67-6.27 (m, 2H), 4.48-4.19 (m, 5H), 4.15-3.91 (m, 2H), 3.74-3.63 (m, 1H), 3.61-3.51 (m, 1H), 2.58-2.36 (m, 9H), 2.35-2.21 (m, 2H), 2.09-1.72 (m, 8H), 0.76-0.63 (m, 2H), 0.55-0.43 (m, 2H). | 673.5 |
| 156 | 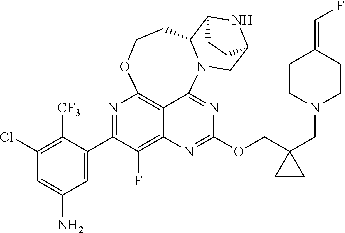 [SMILES: Nc1cc(Cl)c(C(F)(F)F)c(-c2nc3c4c(nc(OCC5(CN6CCC(=CF)CC6)CC5)nc4c2F)N2C[C@H]4CC[C@H](N4)[C@H]2CCO3)c1] | 3-chloro-5-((6aR,7S,10R)-1- fluoro-13-((1-((4- (fluoromethylene) piperidin-1- yl)methyl)cyclopropyl)methoxy)- 5,6,6a,7,8,9,10,11-octahydro-4- oxa-3,11a,12,14,15-pentaaza- 7,10- methanocyclohepta[4,5]cycloocta [1,2,3-de]naphthalen-2-yl)-4- (trifluoromethyl)aniline | 6.86 (s, 1H), 6.62- 6.30 (m, 2H), 4.49- 4.13 (m, 5H), 4.10- 3.82 (m, 2H), 3.72- 3.64 (m, 1H), 3.62- 3.49 (m, 1H), 2.59- 2.37 (m, 6H), 2.34- 2.21 (m, 2H), 2.14- 1.99 (m, 4H), 1.98- 1.68 (m, 4H), 0.78- 0.62 (m, 2H), 0.55- 0.41 (m, 2H). | 692.4 |

Examples 157-194: The following examples were prepared in a manner similar to that in Example 77 by replacing the corresponding substitution or coupling partners. The products were purified by prep-HPLC (Column: Waters Xselect C18, 19*150 mm, 5 μm; Mobile Phase A: H₂O (0.1% NH₃·H₂O+10 mM NH₄HCO₃), Mobile Phase B: MeCN; Flow Rate: 17 mL/min; Detector: UV 214 & 254 nm; Gradient: 45 to 70% B with 0 to 11.0 min, 70 to 90% B with 11.0 to 11.2 min).

| | | | | MS |
|---|---|---|---|---|
| | | | | (ESI, |
| Ex- | | | | m/e) |
| am- | | | 1H NMR (500 MHz, | [M + |
| ple | Structure | Compound Name | CD₃OD) δ | H]⁺ |
| 157 | 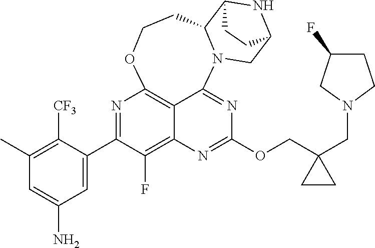 [SMILES: Cc1cc(N)cc(-c2nc3c4c(nc(OCC5(CN6CC[C@H](F)C6)CC5)nc4c2F)N2C[C@H]4CC[C@H](N4)[C@H]2CCO3)c1C(F)(F)F] | 3-((6aR,7S,10R)- 1-fluoro-13-((1-(((S)- 3-fluoropyrrolidin-1- yl)methyl)cyclopropyl) methoxy)- 5,6,6a,7,8,9,10,11- octahydro-4-oxa- 3,11a,12,14,15-pentaaza- 7,10-methano- cyclohepta[4,5]cycloocta [1,2,3-de]naphthalen- 2-yl)-5-methyl-4- (trifluoromethyl)aniline | 6.69 (s, 1H), 6.46-6.44 (m, 1H), 5.20-5.09 (m, 1H), 4.50-3.71 (m, 9H), 3.02-2.56 (m, 6H), 2.42 (s, 3H), 2.19-1.92 (m, 8H), 0.73-0.56 (m, 4H). | 646.5 |
| 158 | 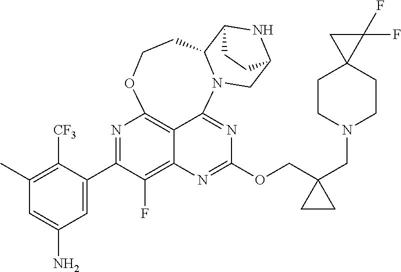 [SMILES: Cc1cc(N)cc(-c2nc3c4c(nc(OCC5(CN6CCC7(CC6)CC7(F)F)CC5)nc4c2F)N2C[C@H]4CC[C@H](N4)[C@H]2CCO3)c1C(F)(F)F] | 3-((6aR,7S,10R)- 13-((1-((1,1- difluoro-6-azaspiro[2.5] octan-6-yl)methyl) cyclopropyl)methoxy)- 1-fluoro- 5,6,6a,7,8,9,10,11- octahydro-4-oxa- 3,11a,12,14,15- pentaaza[7,10] methanocyclo- hepta[4,5]cycloocta[1,2,3- de]naphthalen-2- yl)-5-methyl-4- (trifluoromethyl)aniline | 6.69 (s, 1H), 6.49-6.45 (m, 1H), 4.42-3.66 (m, 9H), 2.68-2.57 (m, 6H), 2.42 (s, 3H), 2.10-1.61 (m, 10H), 1.14-1.08 (m, 2H), 0.74-0.54 (m, 4H). | 704.5 |
| 159 | 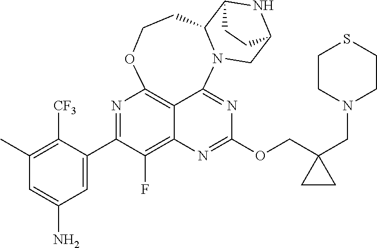 [SMILES: Cc1cc(N)cc(-c2nc3c4c(nc(OCC5(CN6CCSCC6)CC5)nc4c2F)N2C[C@H]4CC[C@H](N4)[C@H]2CCO3)c1C(F)(F)F] | 3-((6aR,7S,10R)- 1-fluoro-13-((1- (thiomorpholinomethyl) cyclopropyl) methoxy)- 5,6,6a,7,8,9,10,11- octahydro-4-oxa- 3,11a,12,14,15- pentaaza-7,10-methano- cyclohepta[4,5]cycloocta [1,2,3-de]naphthalen- 2-yl)-5-methyl-4- (trifluoromethyl)aniline | 6.66 (s, 1H), 6.39 (s, 1H), 4.40-4.31 (m, 3H), 4.24 (s, 1H), 4.04 (s, 1H), 3.65 (s, 1H), 3.59-3.45 (m, 1H), 2.76-2.68 (m, 4H), 2.58-2.53 (m, 4H), 2.44-2.34 (m, 5H), 2.19-1.90 (m, 4H), 1.90-1.69 (m, 4H), 0.68 (s, 2H), 0.46 (s, 2H). | 660.5 |
| 160 | 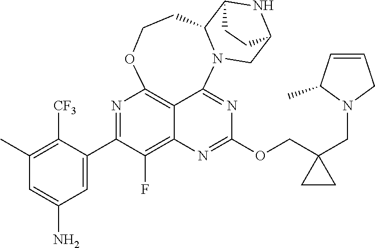 [SMILES: Cc1cc(N)cc(-c2nc3c4c(nc(OCC5(CN6CC=C[C@H]6C)CC5)nc4c2F)N2C[C@H]4CC[C@H](N4)[C@H]2CCO3)c1C(F)(F)F] | 3-((6aR,7S,10R)- 1-fluoro-13- ((1-(((R)-2-methyl- 2,5-dihydro- 1H-pyrrol-1-yl)methyl) cyclopropyl)methoxy)- 5,6,6a,7,8,9,10,11- octahydro-4-oxa- 3,11a,12,14,15-pentaaza- 7,10-methano- cyclohepta[4,5]cycloocta [1,2,3-de]naphthalen- 2-yl)-5-methyl-4- (trifluoromethyl)aniline | 6.66 (s, 1H), 6.39 (s, 1H), 5.73 (s, 1H), 5.61 (s, 1H), 4.73 (d, J = 11.0 Hz, 1H), 4.33 (s, 1H), 4.22 (s, 1H), 4.03 (d, J = 10.7 Hz, 2H), 3.88 (d, J = 12.2 Hz, 1H), 3.63 (s, 1H), 3.53-3.45 (m, 2H), 2.40 (s, 3H), 2.19- 1.68 (m, 9H), 1.35- 1.24 (m, 2H), 1.06 (d, J = 6.5 Hz, 3H), 0.65 (s, 2H), 0.57 (d, J = 8.8 Hz, 1H), 0.46 (d, J = 9.3 Hz, 1H). | 640.5 |
| 161 | 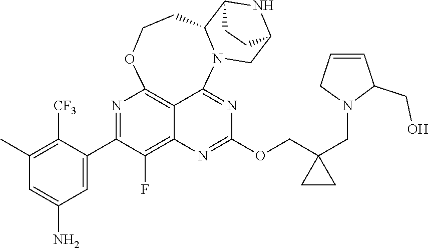 [SMILES: Cc1cc(N)cc(-c2nc3c4c(nc(OCC5(CN6CC=CC6CO)CC5)nc4c2F)N2C[C@H]4CC[C@H](N4)[C@H]2CCO3)c1C(F)(F)F] | (1-((1-((((6aR,7S,10R)- 2-(5-amino-3- methyl-2-(trifluoro- methyl)phenyl)-1-fluoro- 5,6,6a,7,8,9,10,11- octahydro-4-oxa- 3,11a,12,14,15-pentaaza- 7,10-methano- cyclohepta[4,5]cycloocta [1,2,3-de]naphthalen-13- yl)oxy)methyl) cyclopropyl) methyl)-2,5-dihydro- 1H-pyrrol- 2-yl)methanol | 6.69 (s, 1H), 6.53-6.33 (m, 1H), 5.93-5.80 (m, 1H), 5.80-5.69 (m, 1H), 4.85-4.78 (m, 1H), 4.42-4.32 (m, 1H), 4.30-4.23 (m, 1H), 4.14-4.06 (m, 1H), 4.04-3.96 (m, 2H), 3.80-3.70 (m, 1H), 3.67-3.58 (m, 2H), 3.57-3.52 (m, 1H), 3.49-3.39 (m, 2H), 2.42 (s, 3H), 2.11- 2.00 (m, 2H), 1.90- 1.80 (m, 2H), 1.44- | 655.7 |
| | (mix of 2 isomers) | | 1.19 (m, 6H), 0.74- | |
| | | | 0.45 (m, 4H). | |
| 162 | 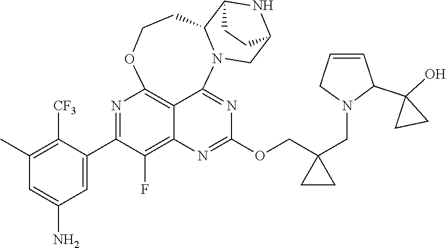 [SMILES: Cc1cc(N)cc(-c2nc3c4c(nc(OCC5(CN6CC=CC6C6(O)CC6)CC5)nc4c2F)N2C[C@H]4CC[C@H](N4)[C@H]2CCO3)c1C(F)(F)F] | 1-(1-((1- ((((6aR,7S,10R)-2-(5- amino-3-methyl-2- (trifluoromethyl) phenyl)-1- fluoro-5,6,6a,7,8,9,10,11- octahydro-4-oxa- 3,11a,12,14,15- pentaaza-7,10-methano- cyclohepta[4,5]cycloocta [1,2,3-de]naphthalen-13- yl)oxy)methyl) cyclopropyl) methyl)-2,5-dihydro- 1H-pyrrol- | 6.66 (s, 1H), 6.53-6.30 (m, 1H), 5.90-5.79 (m, 1H), 5.76-5.64 (m, 1H), 4.38-4.20 (m, 2H), 4.13-3.86 (m, 4H), 3.74 (s, 1H), 3.66- 3.52 (m, 2H), 3.47- 3.35 (m, 1H), 2.39 (s, 3H), 2.25-1.75 (m, 8H), 1.35-1.24 (m, 3H), 0.73-0.60 (m, 3H), 0.54-0.45 (m, 4H). | 682.5 |
| | (mix of 2 isomers) | 2-yl)cyclopropan-1-ol | | |
| 163 | 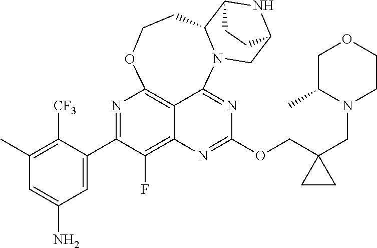 [SMILES: Cc1cc(N)cc(-c2nc3c4c(nc(OCC5(CN6CCOC[C@H]6C)CC5)nc4c2F)N2C[C@H]4CC[C@H](N4)[C@H]2CCO3)c1C(F)(F)F] | 3-((6aR,7S,10R)- 1-fluoro-13- ((1-(((R)-3- methylmorpholino) methyl) cyclopropyl)methoxy)- 5,6,6a,7,8,9,10,11- octahydro-4-oxa- 3,11a,12,14,15-pentaaza- 7,10-methano- cyclohepta[4,5]cycloocta [1,2,3-de]naphthalen- 2-yl)-5-methyl-4- (trifluoromethyl)aniline | 6.59 (s, 1H), 6.46 (s, 1H), 4.69 (s, 1H), 4.31 (s, 2H), 4.07-3.85 (m, 3H), 3.77-3.69 (m, 1H), 3.51-3.06 (m, 3H), 3.35 (s, 1H), 3.14 (s, 1H), 2.99 (d, J = 12.3 Hz, 1H), 2.43 (d, J = 2.1 Hz, 3H), 2.35 (s, 1H), 2.18 (s, 1H), 2.04 (d, J = 6.1 Hz, 4H), 1.54 (s, 3H), 1.22 (s, 2H), 0.88 (d, J = 6.0 Hz, 3H), 0.72-0.53 (m, 2H), 0.52-0.31 (m, 2H). | 658.5 |
| 164 | 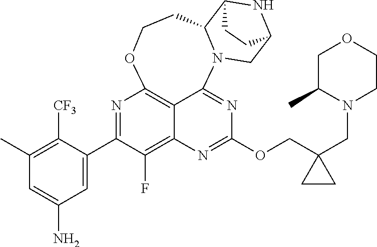 [SMILES: Cc1cc(N)cc(-c2nc3c4c(nc(OCC5(CN6CCOC[C@@H]6C)CC5)nc4c2F)N2C[C@H]4CC[C@H](N4)[C@H]2CCO3)c1C(F)(F)F] | 3-((6aR,7S,10R)- 1-fluoro-13- ((1-(((S)-3- methylmorpholino) methyl) cyclopropyl)methoxy)- 5,6,6a,7,8,9,10,11- octahydro-4-oxa- 3,11a,12,14,15-pentaaza- 7,10-methano- cyclohepta[4,5]cycloocta [1,2,3-de]naphthalen- 2-yl)-5-methyl-4- (trifluoromethyl)aniline | 6.58 (s, 1H), 6.46 (s, 1H), 4.63 (d, J = 10.5 Hz, 1H), 4.31 (s, 2H), 4.05 (d, J = 10.6 Hz, 1H), 3.89 (s, 2H), 3.72 (d, J = 11.0 Hz, 2H), 3.64-3.48 (m, 3H), 3.30 (d, J = 12.6 Hz, 1H), 3.13 (s, 1H), 2.98 (d, J = 11.8 Hz, 1H), 2.43 (d, J = 2.1 Hz, 3H), 2.35 (s, 1H), 2.24-2.11 (m, 2H), 1.99-1.86 (m, 3H), 1.65 (d, J = 13.3 Hz, 2H), 1.37-1.16 (m, | 658.5 |
| | | | 2H), 0.96-0.83 (m, 3H), | |
| | | | 0.71-0.57 (d, J = 14.0 | |
| | | | Hz, 2H), 0.53-0.35 (m, | |
| | | | 2H). | |
| 165 | 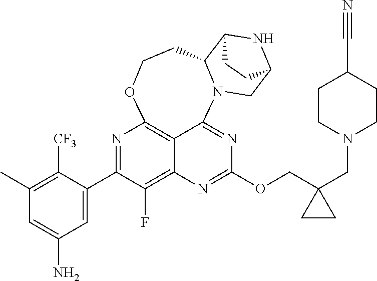 [SMILES: Cc1cc(N)cc(-c2nc3c4c(nc(OCC5(CN6CCC(C#N)CC6)CC5)nc4c2F)N2C[C@H]4CC[C@H](N4)[C@H]2CCO3)c1C(F)(F)F] | 1-((1- ((((6aR,7S,10R)-2-(5- amino-3-methyl-2- (trifluoromethyl) phenyl)-1-fluoro- 5,6,6a,7,8,9,10,11- octahydro-4-oxa- 3,11a,12,14,15- pentaaza-7,10-methano- cyclohepta[4,5]cycloocta [1,2,3-de]naphthalen-13- yl)oxy)methyl) cyclopropyl) methyl)piperidine- 4-carbonitrile | 6.58 (s, 1H), 6.48 (s, 1H), 4.31 (s, 4H), 4.04 (s, 1H), 3.91 (s, 3H), 3.73 (s, 1H), 3.65-3.37 (m, 2H), 2.70 (s, 2H), 2.54 (s, 1H), 2.43 (d, J = 2.1 Hz, 4H), 2.31-2.20 (m, 2H), 1.96-1.17 (m, 9H), 1.28-1.17 (m, 1H), 0.66 (s, 2H), 0.41 (s, 2H). | 667.5 |
| 166 | 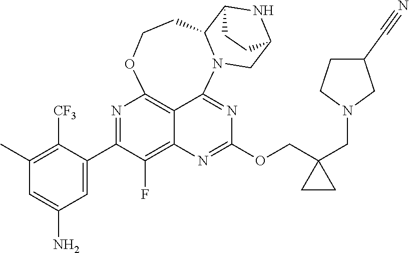 [SMILES: Cc1cc(N)cc(-c2nc3c4c(nc(OCC5(CN6CCC(C#N)C6)CC5)nc4c2F)N2C[C@H]4CC[C@H](N4)[C@H]2CCO3)c1C(F)(F)F] | 1-((1- ((((6aR,7S,10R)-2-(5- amino-3-methyl-2- (trifluoromethyl) phenyl)-1- fluoro-5,6,6a,7,8,9,10,11- octahydro-4-oxa- 3,11a,12,14,15- pentaaza-7,10-methano- cyclohepta[4,5]cycloocta [1,2,3-de]naphthalen-13- yl)oxy)methyl) cyclopropyl) methyl)pyrrolidine- 3-carbonitrile | 6.66 (s, 1H), 6.39 (s, 1H), 4.49 (d, J = 10.5 Hz, 1H), 4.36-4.18 (m, 4H), 4.05 (s, 2H), 3.66 (s, 1H), 3.51 (s, 1H), 3.09 (d, J = 10.7 Hz, 2H), 2.83-2.75 (m, 3H), 2.56-2.49 (m, 3H), 2.39 (s, 3H), 2.17 (s, 1H), 2.02-1.91 (m, 3H), 1.86-1.74 (m, 3H), 0.69 (s, 2H), 0.49 (s, 2H). | 653 |
| | (mix of 2 isomers) | | | |
| 167 | 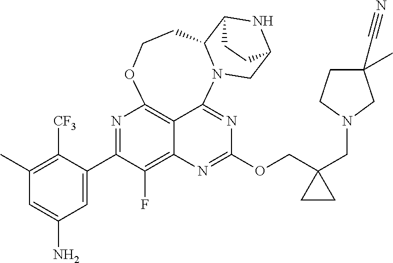 | 1-((1- ((((6aR,7S,10R)-2-(5- amino-3-methyl-2- (trifluoromethyl) phenyl)-1- fluoro-5,6,6a,7,8,9,10,11- octahydro-4-oxa- 3,11a,12,14,15- pentaaza-7,10-methano- cyclohepta[4,5]cycloocta [1,2,3-de]naphthalen-13- yl)oxy)methyl) cyclopropyl) methyl)-3- methylpyrrolidine-3- carbonitrile | 6.66 (s, 1H), 6.41 (d, J = 22.2 Hz, 1H), 4.50 (dd, J = 10.8, 7.5 Hz, 1H), 4.33 (dd, J = 10.8, 5.7 Hz, 2H), 4.23 (s, 1H), 4.05 (s, 1H), 3.67 (s, 1H), 3.51 (s, 1H), 3.07 (t, J = 9.1 Hz, 1H), 2.89-2.83 (m, 1H), 2.57 (d, J = 6.8 Hz, 1H), 2.54-2.49 (m, 2H), 2.45 (d, J = 5.6 Hz, 1H), 2.39 (s, 3H), 2.29- 2.23 (m, 1H), 2.18- 2.02 (m, 2H), 2.01- 1.88 (m, 2H), 1.85- 1.72 (m, 4H), 1.39 (s, | 667 |
| | (mix of 2 isomers) | | 3H), 1.26 (s, 1H), 0.68 | |
| | | | (s, 3H), 0.48 (s, 3H). | |
| 168 | 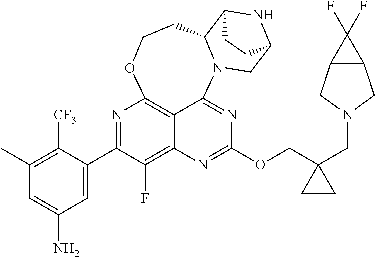 [SMILES: Cc1cc(N)cc(-c2nc3c4c(nc(OCC5(CN6CC7C(C6)C7(F)F)CC5)nc4c2F)N2C[C@H]4CC[C@H](N4)[C@H]2CCO3)c1C(F)(F)F] | 3-((6aR,7S,10R)- 13-((1-((6,6- difluoro-3- azabicyclo[3.1.0]hexan-3- yl)methyl)cyclopropyl) methoxy)- 1-fluoro- 5,6,6a,7,8,9,10,11- octahydro-4-oxa- 3,11a,12,14,15- pentaaza-7,10-methano- cyclohepta[4,5]cycloocta [1,2,3-de]naphthalen- 2-yl)-5-methyl-4- (trifluoromethyl)aniline | 6.58 (s, 1H), 6.46 (s, 1H), 4.28 (d, J = 10.5 Hz, 4H), 4.08-3.86 (m, 3H), 3.77-3.41 (m, 3H), 3.03 (d, J = 9.3 Hz, 2H), 2.80 (d, J = 8.7 Hz, 2H), 2.52-2.37 (m, 5H), 2.25-1.81 (m, 8H), 0.62 (s, 2H), 0.41 (s, 2H). | 676.5 |
| 169 | 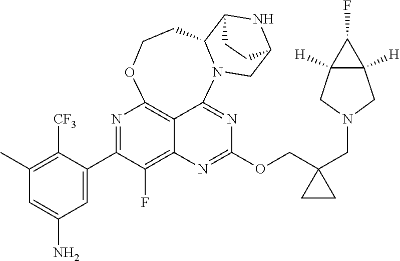 [SMILES: Cc1cc(N)cc(-c2nc3c4c(nc(OCC5(CN6C[C@@H]7[C@@H](F)[C@@H]7C6)CC5)nc4c2F)N2C[C@H]4CC[C@H](N4)[C@H]2CCO3)c1C(F)(F)F] | 3-((6aR,7S,10R)- 1-fluoro-13-((1- (((1R,5S,6S)-6-fluoro-3- azabicyclo[3.1.0]hexan-3- yl)methyl)cyclopropyl) methoxy)- 5,6,6a,7,8,9,10,11- octahydro-4-oxa- 3,11a,12,14,15-pentaaza- 7,10-methano- cyclohepta[4,5]cycloocta [1,2,3-de]naphthalen- 2-yl)-5-methyl-4- (trifluoromethyl)aniline | 6.68 (s, 1H), 6.44 (d, J = 23.2 Hz, 1H), 4.66- 3.81 (m, 8H), 3.78- 3.48 (m, 2H), 3.20- 3.01 (m, 2H), 2.57- 2.26 (m, 7H), 2.22- 1.73 (m, 6H), 1.70 (d, J = 18.3 Hz, 2H), 0.64 (s, 2H), 0.43 (s, 2H). | 658.5 |
| 170 | 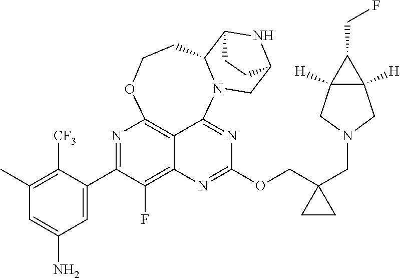 [SMILES: Cc1cc(N)cc(-c2nc3c4c(nc(OCC5(CN6C[C@@H]7[C@@H](CF)[C@@H]7C6)CC5)nc4c2F)N2C[C@H]4CC[C@H](N4)[C@H]2CCO3)c1C(F)(F)F] | 3-((6aR,7S,10R)- 1-fluoro-13- ((1-(((1R,5S,6R)-6- (fluoromethyl)-3- azabicyclo[3.1.0]hexan-3- yl)methyl)cyclopropyl) methoxy)- 5,6,6a,7,8,9,10,11- octahydro-4-oxa- 3,11a,12,14,15-pentaaza- 7,10-methano- cyclohepta[4,5]cycloocta [1,2,3-de]naphthalen- 2-yl)-5-methyl-4- (trifluoromethyl)aniline | 6.68 (s, 1H), 6.47-6.41 (m, 1H), 4.71-4.56 (m, 2H), 4.47-4.06 (m, 9H), 3.69-3.43 (m, 2H), 3.19-3.04 (m, 2H), 2.52- 2.30 (m, 7H), 2.09-1.72 (m, 6H), 1.42-1.30 (m, 3H), 0.65-0.44 (m, 4H). | 672.5 |
| 171 | 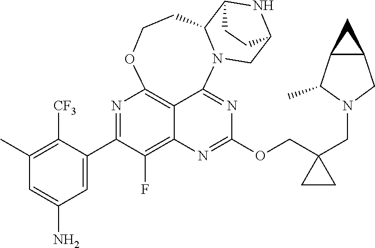 [SMILES: Cc1cc(N)cc(-c2nc3c4c(nc(OCC5(CN6C[C@@H]7C[C@@H]7[C@H]6C)CC5)nc4c2F)N2C[C@H]4CC[C@H](N4)[C@H]2CCO3)c1C(F)(F)F] | 3-((6aR,7S,10R)- 1-fluoro-13-((1- (((1S,2R,5R)-2-methyl-3- azabicyclo[3.1.0]hexan-3- yl)methyl)cyclopropyl) methoxy)- 5,6,6a,7,8,9,10,11- octahydro-4-oxa- 3,11a,12,14,15-pentaaza- 7,10-methano- cyclohepta[4,5]cycloocta [1,2,3-de]naphthalen- 2-yl)-5-methyl-4- (trifluoromethyl)aniline | 6.66 (s, 1H), 6.40 (s, 1H), 4.50 (d, J = 9.3 Hz, 1H), 4.34 (s, 1H), 4.24 (s, 1H), 4.12 (d, J = 9.6 Hz, 1H), 4.08-4.03 (m, 1H), 3.66 (s, 1H), 3.57- 3.50 (m, 1H), 3.03- 2.98 (m, 1H), 2.93 (d, J = 12.5 Hz, 1H), 2.86- 2.81 (m, 1H), 2.64- 2.58 (m, 1H), 2.39 (s, 3H), 2.23-2.01 (m, 4H), 1.89-1.73 (m, 4H), 1.33-1.30 (m, 1H), 1.26 (s, 1H), 1.11 (s, 1H), 0.89 (s, 3H), | 654.6 |
| | | | 0.59 (s, 2H), 0.54 (s, | |
| | | | 1H), 0.41 (s, 2H), 0.36- | |
| | | | 0.32 (m, 1H). | |
| 172 | 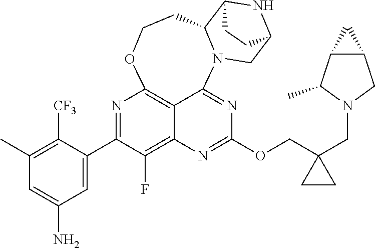 [SMILES: Cc1cc(N)cc(-c2nc3c4c(nc(OCC5(CN6C[C@H]7C[C@H]7[C@H]6C)CC5)nc4c2F)N2C[C@H]4CC[C@H](N4)[C@H]2CCO3)c1C(F)(F)F] | 3-((6aR,7S,10R)- 1-fluoro-13-((1- (((1R,2R,5S)-2-methyl-3- azabicyclo[3.1.0]hexan-3- yl)methyl)cyclopropyl) methoxy)- 5,6,6a,7,8,9,10,11- octahydro-4-oxa- 3,11a,12,14,15-pentaaza- 7,10-methano- cyclohepta[4,5]cycloocta [1,2,3-de]naphthalen- 2-yl)-5-methyl-4- (trifluoromethyl)aniline | 6.66 (s, 1H), 6.40 (s, 1H), 4.34 (s, 1H), 4.23 (s, 1H), 4.06 (s, 1H), 3.82-3.76 (m, 1H), 3.66 (s, 1H), 3.56-3.49 (m, 1H), 2.51 (s, 1H), 2.40 (s, 3H), 2.26 (s, 1H), 2.13-1.98 (m, 3H), 1.97-1.63 (m, 6H), 1.44-1.32 (m, 2H), 1.27 (s, 3H), 0.88 (d, J = 5.4 Hz, 3H), 0.59 (s, 2H), 0.51 (s, 2H), 0.35 (s, 1H), 0.13 (s, 1H). | 654.5 |
| 173 | 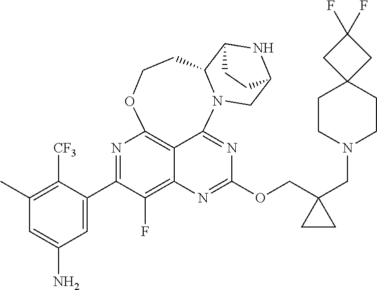 [SMILES: Cc1cc(N)cc(-c2nc3c4c(nc(OCC5(CN6CCC7(CC6)CC(F)(F)C7)CC5)nc4c2F)N2C[C@H]4CC[C@H](N4)[C@H]2CCO3)c1C(F)(F)F] | 3-((6aR,7S,10R)- 13-((1-((2,2- difluoro-7-azaspiro[3.5] nonan-7-yl)methyl) cyclopropyl)methoxy)- 1-fluoro- 5,6,6a,7,8,9,10,11- octahydro-4-oxa- 3,11a,12,14,15- pentaaza[7,10] methanocyclohepta [4,5]cycloocta[1,2,3- de]naphthalen-2-yl)- 5-methyl-4- (trifluoromethyl)aniline | 6.68 (s, 1H), 6.42-6.41 (m, 1H), 4.39-4.07 (m, 7H), 3.68-3.57 (m, 2H), 2.65-2.09 (m, 13H), 2.31-1.64 (m, 10H), 0.72-0.51 (m, 4H). | 718.5 |
| 174 | 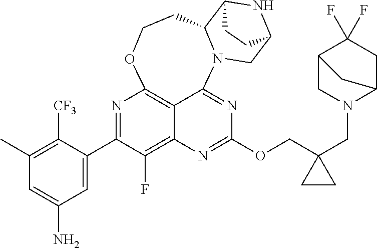 [SMILES: Cc1cc(N)cc(-c2nc3c4c(nc(OCC5(CN6CC7CC6CC7(F)F)CC5)nc4c2F)N2C[C@H]4CC[C@H](N4)[C@H]2CCO3)c1C(F)(F)F] | 3-((6aR,7S,10R)-13-((1- ((5,5-difluoro-2- azabicyclo[2.2.1]heptan-2- yl)methyl)cyclopropyl) methoxy)- 1-fluoro- 5,6,6a,7,8,9,10,11- octahydro-4-oxa- 3,11a,12,14,15- pentaaza-7,10-methano- cyclohepta[4,5]cycloocta [1,2,3-de]naphthalen- 2-yl)-5-methyl-4- (trifluoromethyl)aniline | 6.68 (s, 1H), 6.42-6.41 (m, 1H), 4.47-4.07 (m, 7H), 3.70-3.43 (m, 2H), 2.81-2.57 (m, 5H), 2.42 (s, 3H), 2.11-1.72 (m, 11H), 0.67-0.51 (m, 4H). | 690.5 |
| 175 | 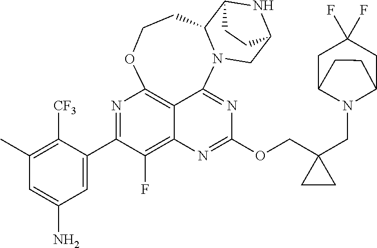 [SMILES: Cc1cc(N)cc(-c2nc3c4c(nc(OCC5(CN6C7CCC6CC(F)(F)C7)CC5)nc4c2F)N2C[C@H]4CC[C@H](N4)[C@H]2CCO3)c1C(F)(F)F] | 3-((6aR,7S,10R)-13-((1- (((1R,5S)-3,3-difluoro-8- azabicyclo[3.2.1]octan-8- yl)methyl)cyclopropyl) methoxy)- 1-fluoro- 5,6,6a,7,8,9,10,11- octahydro-4-oxa- 3,11a,12,14,15- pentaaza-7,10-methano- cyclohepta[4,5]cycloocta [1,2,3-de]naphthalen- 2-yl)-5-methyl-4- (trifluoromethyl)aniline | 6.68 (s, 1H), 6.42-6.41 (m, 1H), 4.52-4.07 (m, 7H), 3.70-3.38 (m, 4H), 2.55-2.45 (m, 2H), 2.42 (s, 3H), 2.11-1.72 (m, 14H), 0.67-0.51 (m, 4H). | 704.5 |
| 176 | 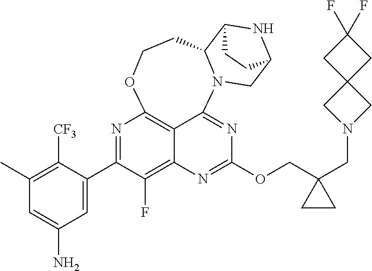 [SMILES: Cc1cc(N)cc(-c2nc3c4c(nc(OCC5(CN6CC7(C6)CC(F)(F)C7)CC5)nc4c2F)N2C[C@H]4CC[C@H](N4)[C@H]2CCO3)c1C(F)(F)F] | 3-((6aR,7S,10R)- 13-((1-((6,6-difluoro- 2-azaspiro[3.3]heptan- 2-yl)methyl) cyclopropyl)methoxy)- 1-fluoro- 5,6,6a,7,8,9,10,11- octahydro-4-oxa- 3,11a,12,14,15- pentaaza[7,10] methanocyclohepta [4,5]cycloocta[1,2,3- de]naphthalen-2-yl)- 5-methyl-4- (trifluoromethyl)aniline | 6.69 (s, 1H), 6.45-6.41 (m, 1H), 4.32-3.63 (m, 13H), 2.89-2.65 (m, 6H), 2.42 (s,3H), 2.10-1.85 (m,6H), 0.73-0.66 (m, 4H). | 690.5 |
| 177 | 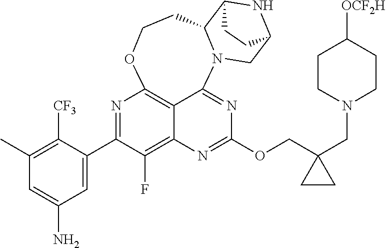 | 3-((6aR,7S,10R)- 1-fluoro-13- ((1-((4-(trifluoro- methoxy)piperidin-1- yl)methyl)cyclopropyl) methoxy)- 5,6,6a,7,8,9,10,11- octahydro-4-oxa- 3,11a,12,14,15-pentaaza- 7,10-methano- cyclohepta[4,5]cycloocta [1,2,3-de]naphthalen- 2-yl)-5-methyl-4- (trifluoromethyl)aniline | 6.67 (s, 1H), 6.41 (s, 1H), 4.37-4.24 (m, 3H), 4.14-3.99 (m, 2H), 3.78-3.57 (m, 2H), 2.90-2.77 (m, 2H), 2.54-2.23 (m, 7H), 2.17-1.99 (m, 2H), 1.96-1.83 (m, 4H), 1.77-1.69 (m, 2H), 1.35-1.19 (m, 2H), 0.70 (s, 2H), 0.49 (s, 2H). | 726.6 |
| 178 | 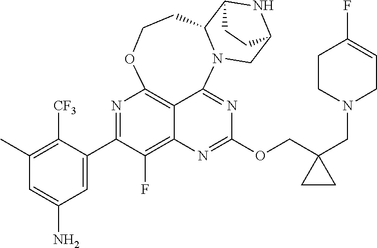 [SMILES: Cc1cc(N)cc(-c2nc3c4c(nc(OCC5(CN6CC=C(F)CC6)CC5)nc4c2F)N2C[C@H]4CC[C@H](N4)[C@H]2CCO3)c1C(F)(F)F] | 3-((6aR,7S,10R)- 1-fluoro-13- ((1-((4-fluoro-3,6- dihydropyridin-1(2H)- yl)methyl)cyclopropyl) methoxy)- 5,6,6a,7,8,9,10,11- octahydro-4-oxa- 3,11a,12,14,15-pentaaza- 7,10-methano- cyclohepta[4,5]cycloocta [1,2,3-de]naphthalen- 2-yl)-5-methyl-4- (trifluoromethyl)aniline | 6.68 (s, 1H), 6.46-6.41 (m, 1H), 5.13-5.10 (m, 1H), 4.40-3.53 (m, 9H), 3.03-2.53 (m, 6H), 2.42 (s, 3H), 2.19-1.75 (m, 8H), 0.72-0.51 (m, 4H). | 658.5 |
| 179 | 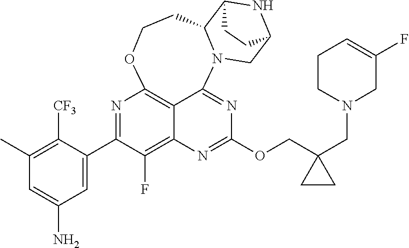 [SMILES: Cc1cc(N)cc(-c2nc3c4c(nc(OCC5(CN6CCC=C(F)C6)CC5)nc4c2F)N2C[C@H]4CC[C@H](N4)[C@H]2CCO3)c1C(F)(F)F] | 3-((6aR,7S,10R)- 1-fluoro-13- ((1-((5-fluoro-3,6- dihydropyridin-1(2H)- yl)methyl)cyclopropyl) methoxy)- 5,6,6a,7,8,9,10,11- octahydro-4-oxa- 3,11a,12,14,15-pentaaza- 7,10-methano- cyclohepta[4,5]cycloocta [1,2,3-de]naphthalen- 2-yl)-5-methyl-4- (trifluoromethyl)aniline | 6.69 (s, 1H), 6.45-6.44 (m, 1H), 5.17-5.14 (m, 1H), 4.64-4.80 (m, 9H), 3.10-3.06 (m, 2H), 2.57- 3.42 (m, 7H), 2.29-1.80 (m, 8H), 0.74-0.53 (m, 4H). | 658.5 |
| 180 | 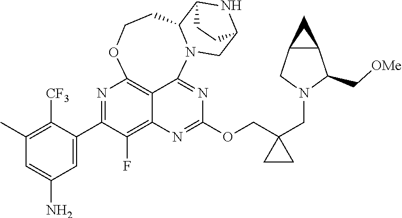 [SMILES: COC[C@@H]1[C@@H]2C[C@@H]2CN1CC1(COc2nc3c4c(nc(-c5cc(N)cc(C)c5C(F)(F)F)c(F)c4n2)OCC[C@@H]2[C@@H]4CC[C@H](CN32)N4)CC1] | 3-((6aR,7S,10R)- 1-fluoro-13- ((1-(((1R,2S,5S)-2- (methoxymethyl)-3- azabicyclo[3.1.0]hexan-3- yl)methyl)cyclopropyl) methoxy)- 5,6,6a,7,8,9,10,11- octahydro-4-oxa- 3,11a,12,14,15-pentaaza- 7,10-methano- cyclohepta[4,5]cycloocta [1,2,3-de]naphthalen- 2-yl)-5-methyl-4- | 6.66 (s, 1H), 6.52-6.30 (m, 1H), 4.95-4.87 (m, 1H), 4.68-4.40 (m, 1H), 4.40-4.29 (m, 1H), 4.28-4.18 (m, 1H), 4.17-3.81 (m, 2H), 3.76-3.60 (m, 2H), 3.57-3.38 (m, 2H), 3.14 (s, 3H), 3.03- 2.86 (m, 1H), 2.73- 2.63 (m, 1H), 2.39 (s, 3H), 2.33-2.26 (m, 1H), 2.18-1.95 (m, 3H), 1.95-1.64 (m, | 684.6 |
| | | (trifluoromethyl)aniline | 4H), 1.61-1.32 (m, | |
| | | | 4H), 0.64-0.53 (m, | |
| | | | 2H), 0.52-0.41 (m, | |
| | | | 2H), 0.37-0.29 (m, | |
| | | | 1H), 0.23-0.15 (m, | |
| | | | 1H). | |
| 181 | 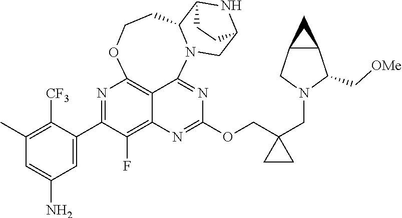 [SMILES: COC[C@H]1[C@@H]2C[C@@H]2CN1CC1(COc2nc3c4c(nc(-c5cc(N)cc(C)c5C(F)(F)F)c(F)c4n2)OCC[C@@H]2[C@@H]4CC[C@H](CN32)N4)CC1] | 3-((6aR,7S,10R)- 1-fluoro-13- ((1-(((1R,2R,5S)-2- (methoxymethyl)-3- azabicyclo[3.1.0]hexan-3- yl)methyl)cyclopropyl) methoxy)- 5,6,6a,7,8,9,10,11- octahydro-4-oxa- 3,11a,12,14,15-pentaaza- 7,10-methano- cyclohepta[4,5]cycloocta [1,2,3-de]naphthalen- 2-yl)-5-methyl-4- | 6.69 (s, 1H), 6.54-6.32 (m, 1H), 4.44-4.25 (m, 4H), 4.22-3.96 (m, 2H), 3.83 (s, 1H), 3.77- 3.66 (m, 1H), 3.46- 3.36 (m, 1H), 3.25 (s, 3H), 3.10-2.74 (m, 4H), 2.69-2.49 (m, 1H), 2.42 (s, 3H), 2.28- 2.02 (m, 3H), 1.98- 1.79 (m, 3H), 1.49- 1.22 (m, 4H), 0.70- 0.53 (m, 3H), 0.51- 0.31 (m, 3H). | 684.6 |
| | | (trifluoromethyl)aniline | | |
| 182 | 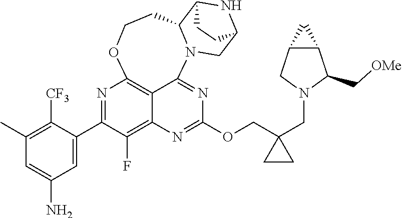 [SMILES: COC[C@@H]1[C@H]2C[C@H]2CN1CC1(COc2nc3c4c(nc(-c5cc(N)cc(C)c5C(F)(F)F)c(F)c4n2)OCC[C@@H]2[C@@H]4CC[C@H](CN32)N4)CC1] | 3-((6aR,7S,10R)- 1-fluoro-13- ((1-(((1S,2S,5R)-2- (methoxymethyl)-3- azabicyclo[3.1.0]hexan-3- yl)methyl)cyclopropyl) methoxy)- 5,6,6a,7,8,9,10,11- octahydro-4-oxa- 3,11a,12,14,15-pentaaza- 7,10-methano- cyclohepta[4,5]cycloocta [1,2,3-de]naphthalen- 2-yl)-5-methyl-4- | 6.67 (s, 1H), 6.42 (s, 1H), 4.52-4.18 (m, 5H), 4.14-4.02 (m, 1H), 3.89-3.81 (m, 1H), 3.79-3.65 (m, 1H), 3.44-3.38 (m, 1H), 3.23 (s, 3H), 3.12- 2.77 (m, 4H), 2.71- 2.46 (m, 1H), 2.40 (d, J = 2.0 Hz, 3H), 2.28- 2.02 (m, 3H), 2.01- 1.83 (m, 3H), 1.48- 1.25 (m, 4H), 0.69- 0.51 (m, 3H), 0.51- | 684.5 |
| | | (trifluoromethyl)aniline | 0.34 (m, 3H). | |
| 183 | 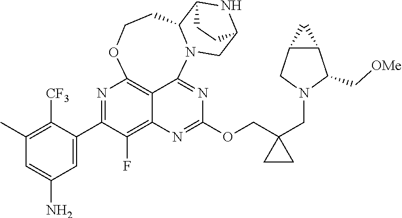 [SMILES: COC[C@H]1[C@H]2C[C@H]2CN1CC1(COc2nc3c4c(nc(-c5cc(N)cc(C)c5C(F)(F)F)c(F)c4n2)OCC[C@@H]2[C@@H]4CC[C@H](CN32)N4)CC1] | 3-((6aR,7S,10R)- 1-fluoro-13- ((1-(((1S,2R,5R)-2- (methoxymethyl)-3- azabicyclo[3.1.0]hexan-3- yl)methyl)cyclopropyl) methoxy)- 5,6,6a,7,8,9,10,11- octahydro-4-oxa- 3,11a,12,14,15-pentaaza- 7,10-methano- cyclohepta[4,5]cycloocta [1,2,3-de]naphthalen- 2-yl)-5-methyl-4- | 6.67 (s, 1H), 6.42 (s, 1H), 4.54 (s, 1H), 4.42 (d, J = 10.8 Hz, 1H), 4.36-4.21 (m, 3H), 4.12-4.00 (m, 1H), 3.87-3.82 (m, 1H), 3.76-3.68 (m, 1H), 3.49-3.39 (m, 2H), 3.25 (s, 3H), 3.14-2.71 (m, 4H), 2.40 (s, 3H), 2.25-2.02 (m, 3H), 2.00-1.82 (m, 3H), 1.39-1.27 (m, 4H), 0.68-0.52 (m, 3H), | 684.5 |
| | | (trifluoromethyl)aniline | 0.51-0.32 (m, 3H). | |
| 184 | 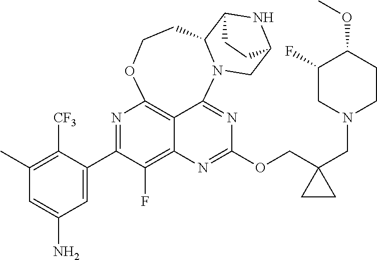 [SMILES: CO[C@@H]1CCN(CC2(COc3nc4c5c(nc(-c6cc(N)cc(C)c6C(F)(F)F)c(F)c5n3)OCC[C@@H]3[C@@H]5CC[C@H](CN43)N5)CC2)C[C@@H]1F] | 3-((6aR,7S,10R)- 1-fluoro-13- ((1-(((3S,4R)-3-fluoro-4- methoxypiperidin-1- yl)methyl)cyclopropyl) methoxy)- 5,6,6a,7,8,9,10,11- octahydro-4-oxa- 3,11a,12,14,15-pentaaza- 7,10-methano- cyclohepta[4,5]cycloocta [1,2,3-de]naphthalen- 2-yl)-5-methyl-4- (trifluoromethyl)aniline | 6.69 (s, 1H), 6.46-6.45 (m, 1H), 4.77-3.89 (m, 11H), 3.37 (s,3H), 3.07- 2.81 (m, 2H), 2.52-1.72 (m, 15H), 0.72-0.50 (m, 4H). | 690.5 |
| 185 | 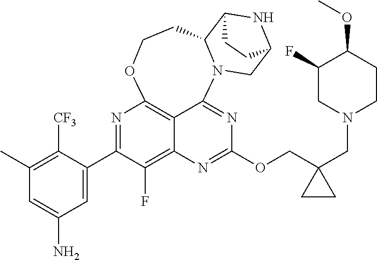 [SMILES: CO[C@H]1CCN(CC2(COc3nc4c5c(nc(-c6cc(N)cc(C)c6C(F)(F)F)c(F)c5n3)OCC[C@@H]3[C@@H]5CC[C@H](CN43)N5)CC2)C[C@H]1F] | 3-((6aR,7S,10R)- 1-fluoro-13- ((1-(((3R,4S)-3-fluoro-4- methoxypiperidin-1- yl)methyl)cyclopropyl) methoxy)- 5,6,6a,7,8,9,10,11- octahydro-4-oxa- 3,11a,12,14,15-pentaaza- 7,10-methano- cyclohepta[4,5]cycloocta [1,2,3-de]naphthalen- 2-yl)-5-methyl-4- (trifluoromethyl)aniline | 6.69 (s, 1H), 6.46-6.44 (m, 1H), 4.77-3.89 (m, 11H), 3.37 (s,3H), 3.04- 2.81 (m, 2H), 2.52-1.72 (m, 15H), 0.72-0.50 (m, 4H). | 690.5 |
| 186 | 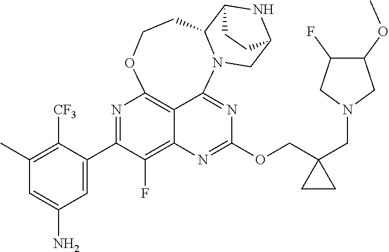 [SMILES: COC1CN(CC2(COc3nc4c5c(nc(-c6cc(N)cc(C)c6C(F)(F)F)c(F)c5n3)OCC[C@@H]3[C@@H]5CC[C@H](CN43)N5)CC2)CC1F] | 3-((6aR,7S,10R)- 1-fluoro-13- ((1-((cis-3-fluoro-4- methoxypyrrolidin-1- yl)methyl)cyclopropyl) methoxy)- 5,6,6a,7,8,9,10,11- octahydro-4-oxa- 3,11a,12,14,15-pentaaza- 7,10-methano- cyclohepta[4,5]cycloocta [1,2,3-de]naphthalen- 2-yl)-5-methyl-4- (trifluoromethyl)aniline (cis-isomer 1) | 6.66 (s, 1H), 6.39 (s, 1H), 4.78 (s, 3H), 4.58 (d, J = 11.0 Hz, 1H), 4.33 (s, 1H), 4.23 (d, J = 11.0 Hz, 2H), 4.03 (s, 1H), 3.83 (d, J = 24.3 Hz, 1H), 3.70 (s, 1H), 3.62-3.54 (m, 1H), 2.93-2.86 (m, 1H), 2.81-2.60 (m, 2H), 2.52 (d, J = 12.3 Hz, 1H), 2.43 (s, 1H), 2.40 (s, 3H), 2.28-2.24 (m, 1H), 2.10-1.80 (m, J = 101.5 Hz, 6H), 1.27 (s, 3H), 0.68 (d, J = 7.0 Hz, | 676.6 |
| | (cis-isomer 1) | | 2H), 0.48 (s, 2H). | |
| 187 | 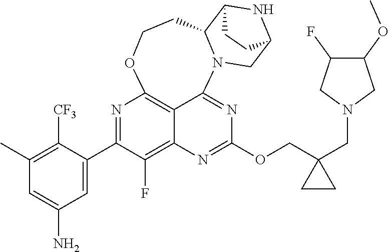 [SMILES: COC1CN(CC2(COc3nc4c5c(nc(-c6cc(N)cc(C)c6C(F)(F)F)c(F)c5n3)OCC[C@@H]3[C@@H]5CC[C@H](CN43)N5)CC2)CC1F] | 3-((6aR,7S,10R)- 1-fluoro-13- ((1-((cis-3-fluoro-4- methoxypyrrolidin-1- yl)methyl)cyclopropyl) methoxy)- 5,6,6a,7,8,9,10,11- octahydro-4-oxa- 3,11a,12,14,15-pentaaza- 7,10-methano- cyclohepta[4,5]cycloocta [1,2,3-de]naphthalen- 2-yl)-5-methyl-4- (trifluoromethyl)aniline (cis-isomer 2) | 6.66 (s, 1H), 6.40 (s, 1H), 4.78 (s, 3H), 4.57 (s, 1H), 4.32 (s, 1H), 4.23 (d, J = 10.9 Hz, 2H), 4.05 (s, 1H), 3.85- 3.78 (m, 1H), 3.71 (s, 1H), 3.61-3.55 (m, 1H), 2.94-2.86 (m, 1H), 2.74-2.65 (m, 1H), 2.53 (d, J = 12.3 Hz, 1H), 2.43 (s, 1H), 2.40 (s, 3H), 2.25 (s, 1H), 2.15-1.69 (m, 8H), 1.40-1.23 (m, 2H), 0.68 (d, J = 8.0 Hz, 2H), 0.48 (s, 2H). | 676.5 |
| | (cis-isomer 2) | | | |
| 188 | 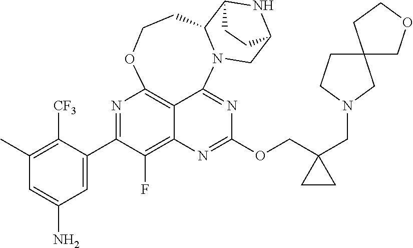 [SMILES: Cc1cc(N)cc(-c2nc3c4c(nc(OCC5(CN6CCC7(CCOC7)C6)CC5)nc4c2F)N2C[C@H]4CC[C@H](N4)[C@H]2CCO3)c1C(F)(F)F] | 3-((6aR,7S,10R)- 13-((1-((2-oxa- 7-azaspiro[4.4]nonan-7- yl)methyl)cyclopropyl) methoxy)- 1-fluoro- 5,6,6a,7,8,9,10,11- octahydro-4-oxa- 3,11a,12,14,15- pentaaza[7,10] methanocyclohepta [4,5]cycloocta[1,2,3- de]naphthalen-2-yl)- 5-methyl-4- (trifluoromethyl)aniline | 6.58 (s, 1H), 6.46 (s, 1H), 4.45-4.26 (m, 4H), 3.90 (s, 3H), 3.83-3.77 (m, 2H), 3.75-3.61 (m, 3H), 3.53-3.42 (m, 2H), 2.78- 2.50 (m, 4H), 2.43 (d, J = 2.1 Hz, 5H), 2.04 (d, J = 6.0 Hz, 3H), 1.92-1.71 (m, 7H), 0.64 (s, 2H), 0.45 (s, 2H). | 684.5 |
| 189 | 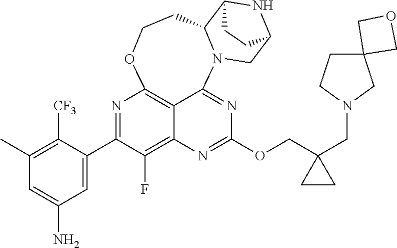 [SMILES: Cc1cc(N)cc(-c2nc3c4c(nc(OCC5(CN6CCC7(COC7)C6)CC5)nc4c2F)N2C[C@H]4CC[C@H](N4)[C@H]2CCO3)c1C(F)(F)F] | 3-((6aR,7S,10R)- 13-((1-((2-oxa- 6-azaspiro[3.4]octan-6- yl)methyl)cyclopropyl) methoxy)-1-fluoro- 5,6,6a,7,8,9,10,11- octahydro-4-oxa- 3,11a,12,14,15- pentaaza[7,10] methanocyclohepta [4,5]cycloocta[1,2,3- de]naphthalen-2-yl)- 5-methyl-4- (trifluoromethyl)aniline | 6.68 (s, 1H), 6.48-6.39 (m, 1H), 4.60-4.57 (m, 2H), 4.57-4.55 (m, 2H), 4.39 (d, J = 3.0 Hz, 2H), 4.37-4.33 (m, 1H), 4.26 (s, 1H), 4.06 (s, 1H), 3.66 (s, 1H), 3.52 (s, 1H), 2.88 (s, 2H), 2.58-2.54 (m, 2H), 2.54-2.44 (m, 2H), 2.42 (d, J = 2.1 Hz, 3H), 2.19-2.04 (m, 4H), 2.01-1.63 (m, 5H), 1.31 (d, J = 23.7 Hz, 1H), 0.70 (s, 2H), | 670.5 |
| | | | 0.52 (s, 2H). | |
| 190 | 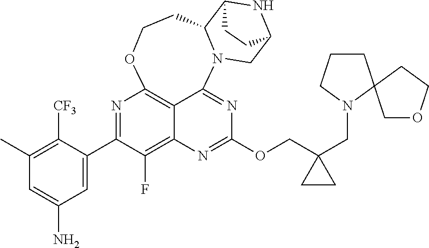 [SMILES: Cc1cc(N)cc(-c2nc3c4c(nc(OCC5(CN6CCCC67CCOC7)CC5)nc4c2F)N2C[C@H]4CC[C@H](N4)[C@H]2CCO3)c1C(F)(F)F] | 3-((6aR,7S,10R)- 13-((1-((7-oxa- 1-azaspiro[4.4]nonan-1- yl)methyl)cyclopropyl) methoxy)-1-fluoro- 5,6,6a,7,8,9,10,11- octahydro-4-oxa- 3,11a,12,14,15- pentaaza[7,10] methanocyclohepta [4,5]cycloocta[1,2,3- de]naphthalen-2-yl)- 5-methyl-4- (trifluoromethyl)aniline | 6.69 (s, 1H), 6.45-6.44 (m, 1H), 4.48-3.64 (m, 13H), 3.21-3.10 (m, 4H), 2.42 (s,3H), 2.18-1.65 (m, 12H), 0.70-0.50 (m, 4H). | 684.5 |
| 191 | 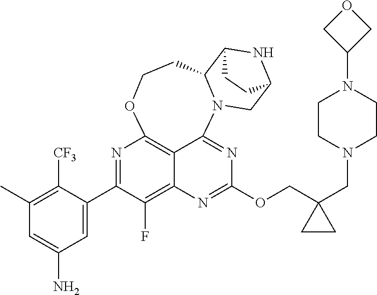 [SMILES: Cc1cc(N)cc(-c2nc3c4c(nc(OCC5(CN6CCN(C7COC7)CC6)CC5)nc4c2F)N2C[C@H]4CC[C@H](N4)[C@H]2CCO3)c1C(F)(F)F] | 3-((6aR,7S,10R)- 1-fluoro-13- ((1-((4-(oxetan-3- yl)piperazin-1- yl)methyl)cyclopropyl) methoxy)- 5,6,6a,7,8,9,10,11- octahydro-4-oxa- 3,11a,12,14,15-pentaaza- 7,10-methano- cyclohepta[4,5]cycloocta [1,2,3-de]naphthalen- 2-yl)-5-methyl-4- (trifluoromethyl)aniline | 6.70 (s, 1H), 6.45-6.44 (m, 1H), 4.64-4.54 (m, 4H), 4.43-3.70 (m,9H), 3.45-3.43 (m, 1H), 2.69- 1.98 (m, 19H), 0.75-0.56 (m, 4H). | 690.5 |
| 192 | 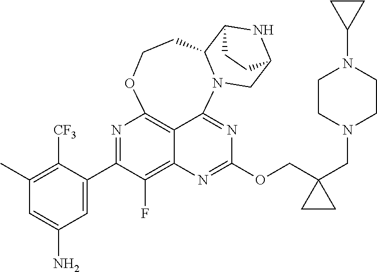 [SMILES: Cc1cc(N)cc(-c2nc3c4c(nc(OCC5(CN6CCN(C7CC7)CC6)CC5)nc4c2F)N2C[C@H]4CC[C@H](N4)[C@H]2CCO3)c1C(F)(F)F] | 3-((6aR,7S,10R)- 13-((1-((4- cyclopropylpiperazin-1- yl)methyl)cyclopropyl) methoxy)-1-fluoro- 5,6,6a,7,8,9,10,11- octahydro-4-oxa- 3,11a,12,14,15- pentaaza-7,10-methano- cyclohepta[4,5]cycloocta [1,2,3-de]naphthalen- 2-yl)-5-methyl-4- (trifluoromethyl)aniline | 6.68 (s, 1H), 6.45-6.41 (m, 1H), 4.53-3.83 (m, 7H), 3.78-3.54 (m, 2H), 2.84-2.28 (m, 13H), 2.10-1.88 (m, 6H), 1.61- 1.57 (m, 1H), 0.73-0.71 (m, 2H), 0.57-0.34 (m, 6H). | 683.6 |
| 193 | 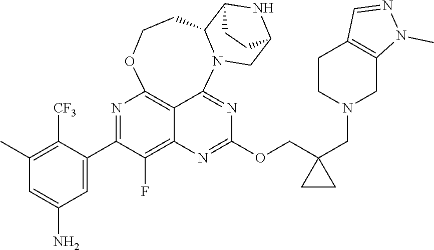 | 3-((6aR,7S,10R)- 1-fluoro-13- ((1-((1-methyl-1,4,6,7- tetrahydro-5H-pyrazolo [4,3-c]pyridin-5- yl)methyl)cyclopropyl) methoxy)- 5,6,6a,7,8,9,10,11- octahydro-4-oxa- 3,11a,12,14,15-pentaaza- 7,10-methano- cyclohepta[4,5]cycloocta [1,2,3-de]naphthalen- 2-yl)-5-methyl-4- (trifluoromethyl)aniline | 7.13 (s, 1H), 6.66 (s, 1H), 6.47 (s, 1H), 4.73-4.16 (m, 5H), 4.14-3.87 (m, 2H), 3.80-3.66 (m, 2H), 3.64-3.52 (m, 4H), 3.46- 3.36 (m, 1H), 2.97- 2.68 (m, 3H), 2.67-2.45 (m, 3H), 2.40 (s, 3H), 2.19-1.95 (m, 3H), 1.91- 1.68 (m, 3H), 0.82- 0.67 (m, 2H), 0.64-0.46 (m, 2H). | 694.5 |
| 194 | 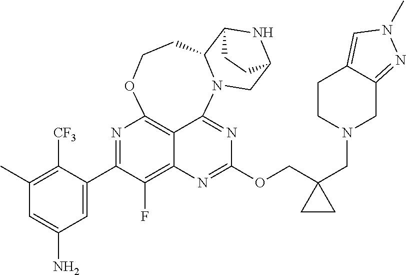 | 3-((6aR,7S,10R)- 1-fluoro-13- ((1-((2-methyl-2,4,6,7- tetrahydro-5H-pyrazolo [4,3-c]pyridin-5- yl)methyl)cyclopropyl) methoxy)- 5,6,6a,7,8,9,10,11- octahydro-4-oxa- 3,11a,12,14,15-pentaaza- 7,10-methano- cyclohepta[4,5]cycloocta [1,2,3-de]naphthalen- 2-yl)-5-methyl-4- (trifluoromethyl)aniline | 7.15 (s, 1H), 6.66 (s, 1H), 6.46 (s, 1H), 4.68- 4.36 (m, 2H), 4.36- 4.18 (m, 2H), 4.15- 3.80 (m, 2H), 3.79- 3.57 (m, 5H), 3.56- 3.39 (m, 2H), 3.07- 2.46 (m, 6H), 2.40 (s, 3H), 2.16-1.92 (m, 3H), 1.91-1.68 (m, 3H), 0.82-0.64 (m, 2H), 0.64-0.48 (m, 2H). | 694.5 |

The following Examples 195-201 were prepared in similar fashion as Example 01 by replacing (2,2-difluorotetrahydro-1H-pyrrolizin-7a(5H)-yl)methanol with the corresponding alkyl alcohols.

| | | | | MS |
|---|---|---|---|---|
| Ex- | | | ¹H NMR (500 MHz, | (ESI, m/e) |
| ample | Structure | Compound Name | CD₃OD) δ | [M + H]⁺ |
| 195 | 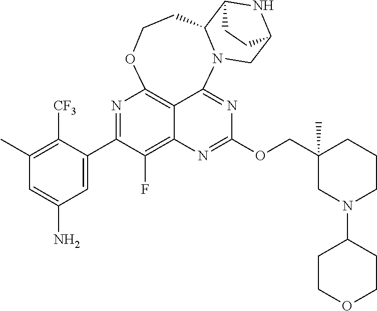 [SMILES: Cc1cc(N)cc(-c2nc3c4c(nc(OC[C@@]5(C)CCCN(C6CCOCC6)C5)nc4c2F)N2C[C@H]4CC[C@H](N4)[C@H]2CCO3)c1C(F)(F)F] | 3-((6aR,7S,10R)-1-fluoro-13- (((S)-3-methyl-1-(tetrahydro-2H- pyran-4-yl)piperidin-3- yl)methoxy)-5,6,6a,7,8,9,10,11- octahydro-4-oxa-3,11a,12,14,15- pentaaza-7,10- methanocyclohepta[4,5]cycloocta [1,2,3-de]naphthalen-2-yl)-5- methyl-4- (trifluoromethyl)aniline | 6.68 (s, 1H), 6.44 (d, J = 21.6 Hz, 1H), 4.58- 3.82 (m, 9H), 3.68 (s, 1H), 3.53 (s, 1H), 3.37 (t, J = 11.3 Hz, 2H), 2.67 (s, 2H), 2.46- 2.42 (m, 6H), 1.64- 1.54 (m, 13H), 1.30 (s, 1H), 1.10 (s, 3H). | 686.6 |
| 196 | 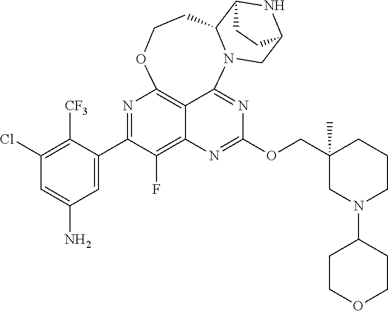 [SMILES: C[C@]1(COc2nc3c4c(nc(-c5cc(N)cc(Cl)c5C(F)(F)F)c(F)c4n2)OCC[C@@H]2[C@@H]4CC[C@H](CN32)N4)CCCN(C2CCOCC2)C1] | 3-chloro-5-((6aR,7S,10R)-1- fluoro-13-(((S)-3-methyl-1- (tetrahydro-2H-pyran-4- yl)piperidin-3-yl)methoxy)- 5,6,6a,7,8,9,10,11- octahydro-4-oxa-3,11a,12,14,15- pentaaza-7,10- methanocyclopenta[4,5] cycloocta[1,2,3-de]naphthalen- 2-yl)-4-(trifluoromethyl) aniline | 6.89 (s, 1H), 6.52 (d, J = 25.7 Hz, 1H), 4.63- 3.90 (m, 9H), 3.86- 3.76 (m, 2H), 3.41 (dd, J = 21.9, 10.7 Hz, 2H), 2.94-2.65 (m, 5H), 2.12-1.67 (m, 13H), 1.44 (s, 1H), 1.15 (s, 3H). | 706.5 |
| 197 | 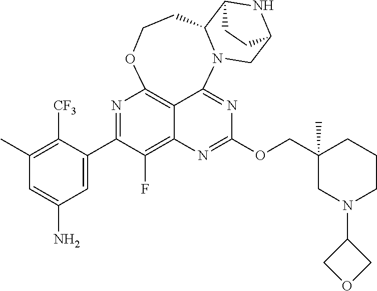 [SMILES: Cc1cc(N)cc(-c2nc3c4c(nc(OC[C@@]5(C)CCCN(C6COC6)C5)nc4c2F)N2C[C@H]4CC[C@H](N4)[C@H]2CCO3)c1C(F)(F)F] | 3-((6aR,7S,10R)-1-fluoro-13- (((S)-3-methyl-1-(oxetan-3- yl)piperidin-3-yl)methoxy)- 5,6,6a,7,8,9,10,11- octahydro-4-oxa-3,11a,12,14,15- pentaaza-7,10- methanocyclohepta[4,5] cycloocta[1,2,3-de] naphthalen-2-yl)-5- methyl-4-(trifluoromethyl) aniline | 6.69 (s, 1H), 6.44 (d, J = 19.7 Hz, 1H), 4.64 (td, J = 6.5, 1.8 Hz, 2H), 4.57-3.83 (m, 9H), 3.72 (s, 1H), 3.57 (s, 1H), 3.42-3.37 (m, 1H), 2.42 (s, 5H), 2.22- 1.57 (m, 11H), 1.35- 1.27 (m, 1H), 1.12 (s, 3H). | 658.6 |
| 198 | 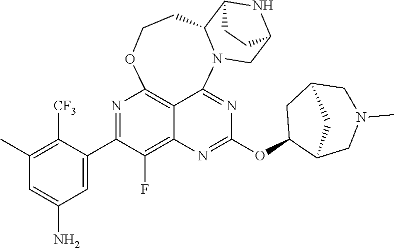 | 3-((6aR,7S,10R)-1-fluoro-13- (((1S,5S,6S)-3-methyl-3- azabicyclo[3.2.1]octan-6-yl) oxy)-5,6,6a,7,8,9,10,11- octahydro-4-oxa-3,11a,12,14,15- pentaaza-7,10- methanocyclohepta[4,5] cycloocta[1,2,3-de] naphthalen-2-yl)-5- methyl-4-(trifluoromethyl) aniline | 6.70 (s, 1H), 6.44 (m, 1H), 5.62 (m, 1H), 4.38-4.30 (m, 2H), 4.15 (s, 2H), 3.84-3.77 (m, 3H), 3.44-3.37 (m, 2H), 3.19-3.14 (m, 2H), 2.89 (s, 3H), 2.79 (s, 1H), 2.70-2.62 (m, 1H), 2.49 (s, 1H), 2.42 (s, 3H), 2.18-1.82 (m, 9H). | 614.5 |
| 199 | 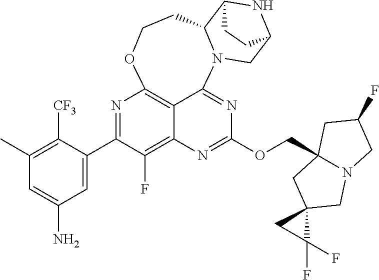 [SMILES: Cc1cc(N)cc(-c2nc3c4c(nc(OC[C@@]56C[C@@H](F)CN5C[C@@]5(CC5(F)F)C6)nc4c2F)N2C[C@H]4CC[C@H](N4)[C@H]2CCO3)c1C(F)(F)F] | 3-((6aR,7S,10R)-1-fluoro-13- (((1S,6′R,7a′R)-2,2,6′- trifluorodihydro-1′H,3′H- spiro[cyclopropane-1,2′- pyrrolizin]-7a′(5′H)-yl)methoxy)- 5,6,6a,7,8,9,10,11- octahydro-4-oxa-3,11a,12,14,15- pentaaza[7,10]methanocyclo- hepta[4,5]cycloocta[1,2,3- de]naphthalen-2-yl)-5- methyl-4-(trifluoromethyl) aniline | 6.68 (s, 1H), 6.47-6.44 (m, 1H), 5.37-5.27 (m, 1H), 4.58-3.94 (m, 7H), 3.65 (s, 1H), 3.56-3.52 (m, 1H), 3.45-3.39 (m, 1H), 3.35-3.32 (m, 1H), 2.96-2.76 (m, 2H), 2.52-2.34 (m, 5H), 2.13-1.73 (m, 8H), 1.51-1.40 (m, 2H). | 694.6 |
| 200 | 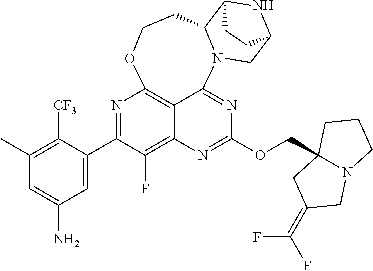 [SMILES: Cc1cc(N)cc(-c2nc3c4c(nc(OC[C@@]56CCCN5CC(=C(F)F)C6)nc4c2F)N2C[C@H]4CC[C@H](N4)[C@H]2CCO3)c1C(F)(F)F] | 3-((6aR,7S,10R)-13-(((S)-2- (difluoromethylene)tetrahydro- 1H-pyrrolizin-7a(5H)- yl)methoxy)-1-fluoro- 5,6,6a,7,8,9,10,11- octahydro-4-oxa-3,11a,12,14,15- pentaaza-7,10- methanocyclohepta[4,5]cycloocta [1,2,3-de]naphthalen-2-yl)-5- methyl-4-(trifluoromethyl) aniline | 6.68 (s, 1H), 6.46- 6.42 (m, 1H), 4.47- 3.90 (m, 7H), 3.80- 3.41 (m, 4H), 3.15- 3.12 (m, 1H), 2.81- 2.49 (m, 3H), 2.42 (s, 3H), 2.12-1.88 (m, 10H) | 662.5 |
| 201 | 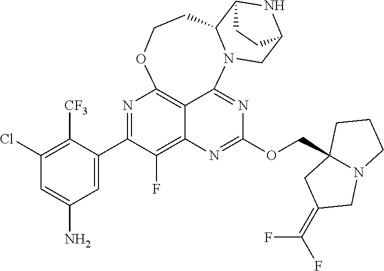 [SMILES: Nc1cc(Cl)c(C(F)(F)F)c(-c2nc3c4c(nc(OC[C@@]56CCCN5CC(=C(F)F)C6)nc4c2F)N2C[C@H]4CC[C@H](N4)[C@H]2CCO3)c1] | 3-chloro-5-((6aR,7S,10R)-13- (((S)-2- (difluoromethylene)tetrahydro- 1H-pyrrolizin-7a(5H)- yl)methoxy)-1-fluoro- 5,6,6a,7,8,9,10,11- octahydro-4-oxa-3,11a,12,14,15- pentaaza-7,10- methanocyclohepta[4,5]cyclo- octa[1,2,3-de]naphthalen-2-yl)- 4-(trifluoromethyl)aniline | 6.68 (s, 1H), 6.49- 6.48 (m, 1H), 4.52- 3.90 (m, 7H), 3.80- 3.41 (m, 4H), 3.15- 3.12 (m, 1H), 2.81- 2.49 (m, 3H), 2.12- 1.88 (m, 10H) | 682.5 |

Examples 202-203: The following examples were prepared in a similar fashion as Example 01 by replacing (2,2-difluorotetrahydro-1H-pyrrolizin-7a(5H)-yl)methanol with the corresponding alkyl alcohol as a mixture of isomers, which was further separated via chiral Prep-HPLC (Column: CHIRALPAK IG, 2×25 cm, 5 μM; Mobile Phase A: n-Hexane (0.5% 2M NH₃-MeOH), Mobile Phase B: EtOH:DCM=1:1; Flow Rate: 20 mL/min; Temperature: 25° C.; Gradient: Isocratic 20% B).

| | | | | MS |
|---|---|---|---|---|
| | | | 1H NMR (500 MHz, | (ESI, m/e) |
| Example | Structure | Compound Name | CD₃OD) δ | [M + H]⁺ |
| 202 | 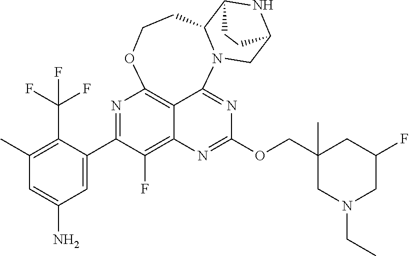 [SMILES: CCN1CC(F)CC(C)(COc2nc3c4c(nc(-c5cc(N)cc(C)c5C(F)(F)F)c(F)c4n2)OCC[C@@H]2[C@@H]4CC[C@H](CN32)N4)C1] | 3-((6aR,7S,10R)-13-((1-ethyl-5- fluoro-3-methylpiperidin-3- yl)methoxy)-1-fluoro- 5,6,6a,7,8,9,10,11- octahydro-4-oxa-3,11a,12,14,15- pentaaza-7,10- methanocyclohepta[4,5]cyclo- octa[1,2,3-de]naphthalen-2- yl)-5-methyl-4- (trifluoromethyl)aniline | 6.69 (s, 1H), 6.46- 6.42 (m, 1H), 4.78- 3.89 (m, 8H), 3.74- 3.57 (m, 2H), 2.87- 2.85 (m, 1H), 2.68- 2.65 (m, 1H), 2.49- 2.42 (s, 5H), 2.31- 2.30 (m, 1H), 2.13- 1.75 (m, 8H), 1.46- 1.42 (m, 1H), 1.18 (s, 3H), 1.07-1.04 (m, 3H). | 648.5 |
| | (elute 1) | | | |
| 203 | 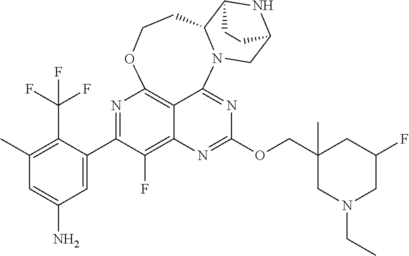 [SMILES: CCN1CC(F)CC(C)(COc2nc3c4c(nc(-c5cc(N)cc(C)c5C(F)(F)F)c(F)c4n2)OCC[C@@H]2[C@@H]4CC[C@H](CN32)N4)C1] | 3-((6aR,7S,10R)-13-((1-ethyl- 5-fluoro-3-methylpiperidin-3- yl)methoxy)-1-fluoro- 5,6,6a,7,8,9,10,11- octahydro-4-oxa-3,11a,12,14,15- pentaaza-7,10- methanocyclohepta[4,5]cyclo- octa[1,2,3-de]naphthalen-2- yl)-5-methyl-4- (trifluoromethyl)aniline | 6.69 (s, 1H), 6.47- 6.41 (m, 1H), 4.73- 3.89 (m, 8H), 3.70- 3.57 (m, 2H), 2.87- 2.85 (m, 1H), 2.66- 2.64 (m, 1H), 2.46- 2.42 (s, 5H), 2.31- 2.30 (m, 1H), 2.14- 1.75 (m, 8H), 1.46- 1.42 (m, 1H), 1.18 (s, 3H), 1.08-1.05 (m, 3H). | 648.5 |
| | (elute 2) | | | |

### Example 204 and 205: 3-((6aR,7S,10R)-1-fluoro-13-(((2R)-2-fluoro-2,3-dihydro-1H-pyrrolizin-7a(5H)-yl)methoxy)-5,6,6a,7,8,9,10,11-octahydro-4-oxa-3,11a,12,14,15-pentaaza-7,10-methanocyclohepta[4,5]cycloocta[1,2,3-de]naphthalen-2-yl)-5-methyl-4-(trifluoromethyl)aniline

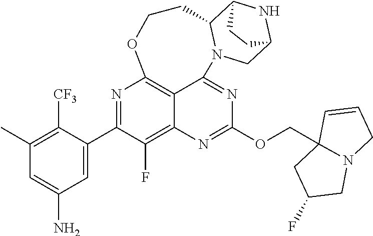 [SMILES: Cc1cc(N)cc(-c2nc3c4c(nc(OCC56C=CCN5C[C@H](F)C6)nc4c2F)N2C[C@H]4CC[C@H](N4)[C@H]2CCO3)c1C(F)(F)F]

### Synthetic Route

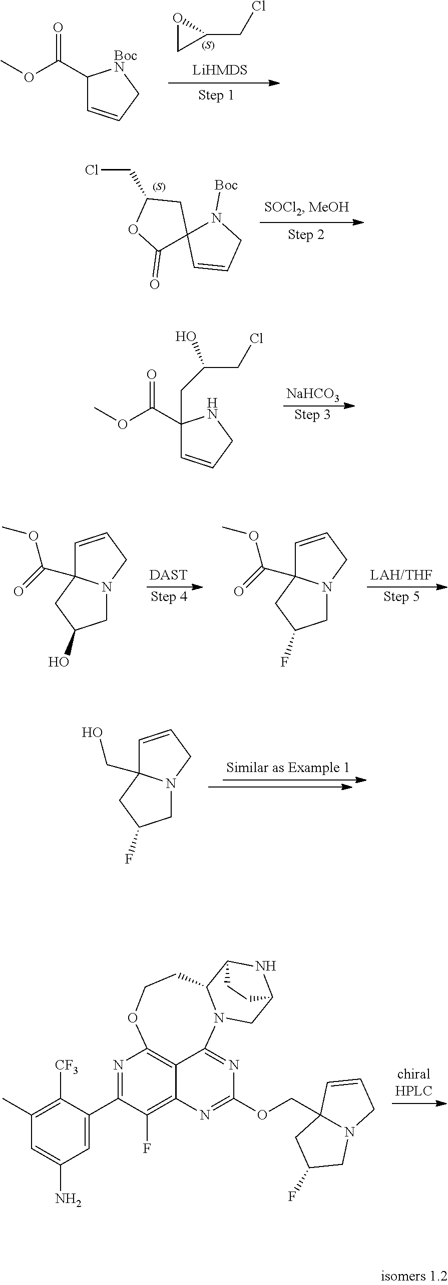

### Step 1: tert-butyl (8S)-8-(chloromethyl)-6-oxo-7-oxa-1-azaspiro[4.4]non-3-ene-1-carboxylate

To a solution of 1-(tert-butyl) 2-methyl 2,5-dihydro-1H-pyrrole-1,2-dicarboxylate (1 g, 4.4 mmol) in THF (0.8 L) was added a solution of LiHMDS (7 mL, 1 M, 7 mmol) dropwise at −78° C. The reaction was stirred at −78° C. for 1 hour, followed by addition of (S)-2-(chloromethyl)oxirane (1.2 g, 13.2 mmol). The reaction was further stirred at −78° C. for 1 hour, and then warmed up to room temperature and stirred for additional 15 hours. The reaction was quenched by addition of saturated NH₄Cl aqueous solution. The resulting mixture was extracted with EtOAc. The combined organic phase was dried over Na₂SO₄, and filtered and concentrated in vacuo. The residue was purified by silica gel column chromatography (dry loading, eluted with 0 to 20% EtOAc in PE) to give the title product (0.6 g). MS (ESI, m/e) [M+Na]⁺ 310.1.

### Step 2: methyl 2-((S)-3-chloro-2-hydroxypropyl)-2,5-dihydro-1H-pyrrole-2-carboxylate

To a solution of tert-butyl (8S)-8-(chloromethyl)-6-oxo-7-oxa-1-azaspiro[4.4]non-3-ene-1-carboxylate (0.6 g, 2.1 mmol) in MeOH (10 mL) was added SOCl₂ (0.1 mL) at room temperature. The resulting mixture was degassed by bubbling nitrogen through, and was stirred at 60° C. for 15 hours. The mixture was then concentrated in vacuo to give the title product (0.56 g), which was used directly in the next step without further purification. MS (ESI, m/e) [M+H]⁺ 220.2.

### Step 3: methyl (2S)-2-hydroxy-2,3-dihydro-1H-pyrrolizine-7a(5H)-carboxylate

To a solution of methyl 2-((R)-3-chloro-2-hydroxypropyl)-2,5-dihydro-1H-pyrrole-2-carboxylate (0.56 g, crude) in CH₃CN (10 mL) was added NaHCO₃ (0.53 g, 6.36.3 mmol) at room temperature. The mixture was degassed by bubbling nitrogen through, and was stirred at 70° C. for 30 min. The resulting mixture was concentrated in vacuo. The residue was purified by silica gel column chromatography (dry load, eluted with 0 to 100% EtOAc in PE) to give the titled product (0.23 g, 28% yield in 3 steps). MS (ESI, m/e) [M+H]⁺ 184.2.

### Step 4: methyl (2R)-2-fluoro-2,3-dihydro-1H-pyrrolizine-7a(5H)-carboxylate

To a solution of methyl (2S)-2-hydroxy-2,3-dihydro-1H-pyrrolizine-7a(5H)-carboxylate (0.23 g, 1.25 mmol) in DCM (5 mL) was added DAST (0.06 g, 0.37 mmol) at 0° C. The mixture was degassed by bubbling nitrogen through, and was then stirred at 0° C. for 1 hour. The resulting mixture was concentrated in vacuo. The residue was purified by silica gel column chromatography (dry load, eluted with 0 to 100% EA in PE) to give the titled product (0.11 g, 47%). MS (ESI, m/e) [M+H]⁺ 186.3.

### Step 5: ((2R)-2-fluoro-2,3-dihydro-1H-pyrrolizin-7a(5H)-yl)methanol

To a solution of methyl (2R)-2-fluoro-2,3-dihydro-1H-pyrrolizine-7a(5H)-carboxylate (0.11 g, 0.6 mmol) in THF (5 mL) was added LiAlH₄ (2.5M, 0.23 mL, 0.6 mmol) at 0° C. The mixture was degassed by bubbling nitrogen through, and was stirred at 0° C. for 20 min. After the reaction was complete as indicated by TLC, the reaction was quenched by addition of Na₂SO₄.10H₂O. The resulting mixture was concentrated in vacuo to give the titled product (90 mg), which was used directly in the next step without further purification. MS (ESI, m/e) [M+H]⁺ 158.1.

Steps 6-8: Examples 204-205 was prepared in similar fashion as Example 01 by replacing (2,2-difluorotetrahydro-1H-pyrrolizin-7a(5H)-yl)methanol with abovementioned ((2R)-2-fluoro-2,3-dihydro-1H-pyrrolizin-7a(5H)-yl)methanol. The compound was further separated via prep-HPLC to give 2 isomers (Column: Waters Xselect C18, 19*150 mm, 5 μm; Mobile Phase A: H₂O (with 0.03% NH₃—H₂O), Mobile Phase B: MeCN; Flow Rate: 17 mL/min; Detector: UV 214 & 254 n; Gradient: 32 to 57% B with 0-11.0 min, 57 to 90% B with 11.0 to 11.2 min).

Examples 204 (first eluting): ¹H NMR (400 MHz, CD₃OD) δ 6.66 (s, 1H), 6.48-3.29 (m, 1H), 5.94 (d, J=5.8 Hz, 1H), 5.82 (d, J=5.8 Hz, 1H), 5.24 (d, J=52.9 Hz, 1H), 4.84-4.79 (m, 1H), 4.46 (d, J=10.3 Hz, 1H), 4.38-4.19 (m, 3H), 4.07 (s, 1H), 3.92 (d, J=16.1 Hz, 1H), 3.76-3.56 (m, 2H), 3.55-3.36 (m, 2H), 2.84-2.66 (m, 1H), 2.46-2.32 (m, 4H), 2.21-1.72 (m, 8H). MS (ESI, m/e) [M+1]⁺ 630.5.

Examples 205 (second eluting): H NMR (400 MHz, CD₃OD) δ 6.66 (s, 1H), 6.49-6.31 (m, 1H), 5.85-5.75 (m, 2H), 5.27 (d, J=54.0 Hz, 1H), 4.81-4.78 (m, 1H), 4.39-4.22 (m, 4H), 4.09-3.95 (m, 2H), 3.74 (d, J=15.3 Hz, 1H), 3.66-3.45 (m, 2H), 3.22-3.19 (m, 2H), 2.40 (s, 3H), 2.30-2.17 (m, 2H), 2.11-1.71 (m, 7H). MS (ESI, m/e) [M+H]⁺ 630.5.

The following Examples 206-217 were prepared in similar fashion as Examples 200-201, 204-205.

| Ex- | | | | MS |
|---|---|---|---|---|
| am- | | | ¹H NMR (500 MHz, | (ESI, m/e) |
| ple | Structure | Compound Name | CD₃OD) δ | [M + H]⁺ |
| 206 | 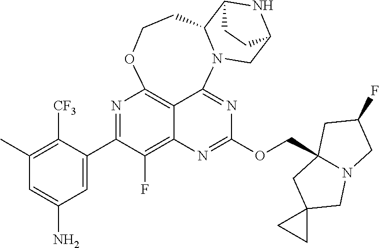 [SMILES: Cc1cc(N)cc(-c2nc3c4c(nc(OC[C@@]56C[C@@H](F)CN5CC5(CC5)C6)nc4c2F)N2C[C@H]4CC[C@H](N4)[C@H]2CCO3)c1C(F)(F)F] | 3-((6aR,7S,10R)-1-fluoro-14- (((6R′,7a′R)-6′-fluorodihydro- 1′H,3′H-spiro[cyclopropane-1,2′- pyrrolizin]-7a′(5′H)-yl)methoxy)- 5,6,6a,7,8,9,10,11- octahydro-[7,10] epiminoazepino[1′,2′:5,6][1,5] oxazocino[2,3,4-ij][2,7] naphthyridin-2-yl)-5- methyl-4-(trifluoromethyl) aniline | 6.68 (s, 1H), 6.44- 6.42 (m, 1H), 5.44- 5.33 (m, 1 H), 4.48- 3.44 (m, 9H), 3.14- 2.52 (m, 4H), 2.42 (s, 3H), 2.06-1.68 (m, 10H), 0.62-0.54 (m, 4H). | 657.5 |
| 207 | 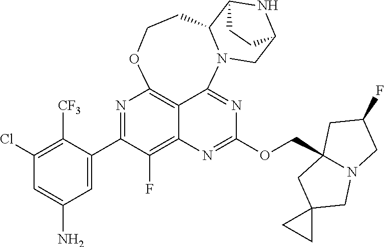 [SMILES: Nc1cc(Cl)c(C(F)(F)F)c(-c2nc3c4c(nc(OC[C@@]56C[C@@H](F)CN5CC5(CC5)C6)nc4c2F)N2C[C@H]4CC[C@H](N4)[C@H]2CCO3)c1] | 3-chloro-5-((6aR,7S,10R)-1- fluoro-13-(((6′R,7a′R)-6′- fluorodihydro-1′H,3′H- spiro[cyclopropane-1,2′- pyrrolizin]-7a′(5′H)-yl) methoxy)- 5,6,6a,7,8,9,10,11- octahydro-4-oxa-3,11a,12,14,15- pentaaza[7,10]methanocyclo- hepta[4,5]cycloocta[1,2,3-de] naphthalen-2-yl)-4- (trifluoromethyl)aniline | 6.69 (s, 1H), 6.55- 6.50 (m, 1H), 5.46- 5.36 (m, 1H), 4.45- 3.44 (m, 9H), 3.11- 2.53 (m, 4H), 2.57- 2.53 (m, 2H), 2.26- 1.83 (m, 8H), 0.66- 0.57 (m, 4H). | 678.5 |
| 208 | 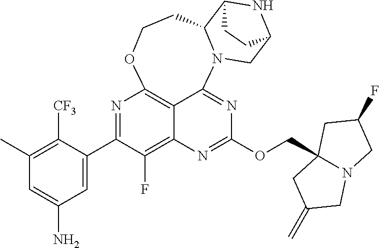 [SMILES: C=C1CN2C[C@H](F)C[C@@]2(COc2nc3c4c(nc(-c5cc(N)cc(C)c5C(F)(F)F)c(F)c4n2)OCC[C@@H]2[C@@H]4CC[C@H](CN32)N4)C1] | 3-((6aR,7S,10R)-1-fluoro-13- (((2R,7aR)-2-fluoro-6- methylenetetrahydro-1H- pyrrolizin-7a(5H)-yl) methoxy)- 5,6,6a,7,8,9,10,11- octahydro-4-oxa-3,11a,12,14,15- pentaaza-7,10- methanocyclohepta[4,5] cycloocta[1,2,3-de] naphthalen-2-yl)-5- methyl-4-(trifluoromethyl) aniline | 6.68 (s, 1H), 6.46- 6.42 (m, 1H), 5.37- 5.25 (m, 1H), 4.99- 4.98 (m, 2H), 4.34- 3.40 (m, 11H), 2.95- 2.84 (m, 2H), 2.52- 2.42 (m, 5H), 2.15- 1.78 (m, 8H). | 644.5 |
| 209 | 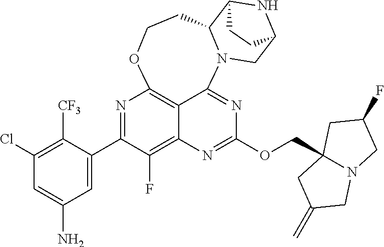 [SMILES: C=C1CN2C[C@H](F)C[C@@]2(COc2nc3c4c(nc(-c5cc(N)cc(Cl)c5C(F)(F)F)c(F)c4n2)OCC[C@@H]2[C@@H]4CC[C@H](CN32)N4)C1] | 3-chloro-5-((6aR,7S,10R)-1- fluoro-13-(((2R,7aR)-2-fluoro- 6-methylenetetrahydro-1H- pyrrolizin-7a(5H)-yl)methoxy)- 5,6,6a,7,8,9,10,11- octahydro-4-oxa-3,11a,12,14,15- pentaaza-7,10- methanocyclohepta[4,5]cyclo- octa[1,2,3-de]naphthalene-2-yl)- 4-(trifluoromethyl)aniline | 6.89-6.88 (m, 1H), 6.55-6.49 (m, 1H), 5.38-5.26 (m, 1H), 5.00 (s, 2H), 4.58 (s, 1H), 4.39-4.24 (m, 4H), 4.15-3.91 (m, 2H), 3.79-3.53 (m, 3H), 3.45-3.37 (m, 1H), 3.27 (s, 1H), 2.97-2.82 (m, 2H), 2.54-2.43 (m, 2H), 2.18-1.79 (m, 7H). | 664.5 |
| 210 | 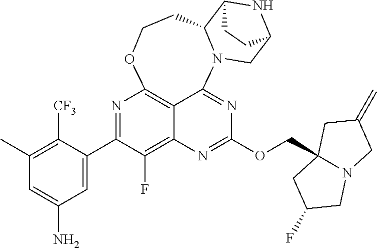 | 3-((6aR,7S,10R)-1-fluoro-13- (((2R,7aS)-2-fluoro-6- methylenetetrahydro-1H- pyrrolizin-7a(5H)-yl)methoxy)- 5,6,6a,7,8,9,10,11- octahydro-4-oxa-3,11a,12,14,15- pentaaza-7,10- methanocyclohepta[4,5]cyclo- octa[1,2,3-de]naphthalen-2-yl)- 5-methyl-4-(trifluoromethyl) aniline | 6.68 (s, 1H), 6.47-6.42 (m, 1H), 5.38-5.27 (m, 1H), 4.96 (s, 2H), 4.36- 4.20 (m, 5H), 4.07-3.97 (m, 2H), 3.77-3.75 (m, 1H), 3.67-3.47 (m, 3H), 3.37-3.28 (m, 1H), 3.17-3.10 (m, 1H), 2.77-2.65 (m, 2H), 2.44-2.20 (m, 5H), 2.11-1.72 (m, 6H). | 644.5 |
| 211 | 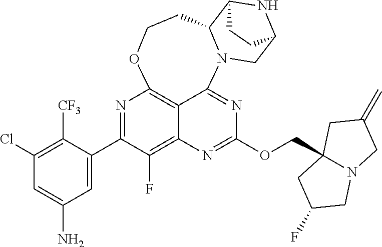 [SMILES: C=C1CN2C[C@H](F)C[C@]2(COc2nc3c4c(nc(-c5cc(N)cc(Cl)c5C(F)(F)F)c(F)c4n2)OCC[C@@H]2[C@@H]4CC[C@H](CN32)N4)C1] | 3-chloro-5-((6aR,7aS,10R)-1- fluoro-13-(((2R,7aS)-2-fluoro- 6-methylenetetrahydro-1H- pyrrolizin-7a(5H)-yl)methoxy)- 5,6,6a,7,8,9,10,11- octahydro-4-oxa-3,11a,12,14,15- pentaaza-7,10- methanocyclohepta[4,5]cyclo- octa[1,2,3-de]naphthalen-2-yl)- 4-(trifluoromethyl)aniline | 6.89-6.88 (m, 1H), 6.56-6.50 (m, 1H), 5.37-5.27 (m, 1H), 4.96 (s, 2H), 4.37-4.20 (m, 5H), 4.08-3.93 (m, 2H), 3.77-3.74 (m, 1H), 3.64-3.51 (m, 3H), 3.36-3.26 (m, 1H), 3.17-3.10 (m, 1H), 2.77-2.67 (m, 2H), 2.40-2.20 (m, 2H), 2.10-1.73 (m, 6H). | 664.5 |
| 212 | 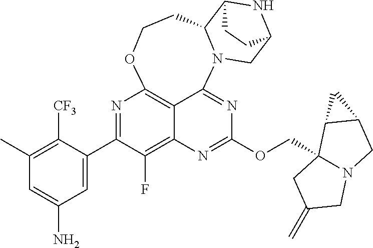 [SMILES: C=C1CN2C[C@H]3C[C@H]3[C@]2(COc2nc3c4c(nc(-c5cc(N)cc(C)c5C(F)(F)F)c(F)c4n2)OCC[C@@H]2[C@@H]4CC[C@H](CN32)N4)C1] | 3-((6aR,7S,10R)-1-fluoro-13- (((1aS,6bR)-5- methylenehexahydrocyclo- propa[a]pyrrolizin-6a(4H)- yl)methoxy)- 5,6,6a,7,8,9,10,11- octahydro-4-oxa-3,11a,12,14,15- pentaaza-7,10-methanocyclohepta [4,5]cycloocta[1,2,3-de] naphthalen-2-yl)-5- methyl-4-(trifluoromethyl) aniline | 6.66 (s, 1H), 6.41 (s, 1H), 5.00 (d, J = 25.6 Hz, 2H), 4.23 (s, 1H), 4.06 (s, 1H), 3.63 (s, 2H), 3.52 (s, 1H), 3.18 (d, J = 9.4 Hz, 3H), 2.87 (d, J = 16.7 Hz, 2H), 2.77 (d, J = 5.7 Hz, 2H), 2.51 (d, J = 17.3 Hz, 2H), 2.40 (s, 3H), 2.06 (s, 2H), 1.79 (s, 2H), 1.59 (s, 2H), 1.26 (d, J = 13.9 Hz, 3H), 0.66 (s, 1H), 0.52 (s, 1H) | 638.6 |
| 213 | 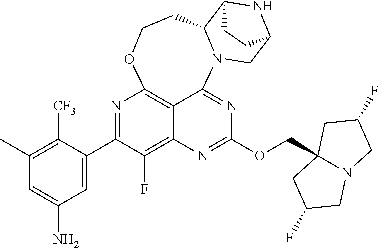 [SMILES: Cc1cc(N)cc(-c2nc3c4c(nc(OC[C@@]56C[C@H](F)CN5C[C@H](F)C6)nc4c2F)N2C[C@H]4CC[C@H](N4)[C@H]2CCO3)c1C(F)(F)F] | 3-((6aR,7S,10R)-13- (((2R,6S,7as)-2,6- difluorotetrahydro-1H- pyrrolizin-7a(5H)-yl) methoxy)-1-fluoro- 5,6,6a,7,8,9,10,11- octahydro-4-oxa-3,11a,12,14,15- pentaaza-7,10- methanocyclohepta[4,5] cycloocta[1,2,3-de]naphthalen- 2-yl)- 5-methyl-4-(trifluoromethyl) aniline | 6.68 (s, 1H), 6.46-6.41 (m, 1H), 5.41-5.30 (m, 7H), 4.24-3.90 (m, 7H), 3.66-3.21 (m, 6H), 2.42-2.32 (m, 7H), 2.15-1.72 (m, 6H). | 650.5 |
| 214 | 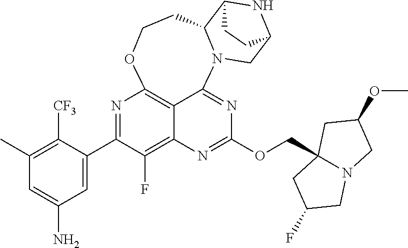 | 3-((6aR,7S,10R)-1-fluoro-13- (((2R,6R,7aS)-2-fluoro-6- methoxytetrahydro-1H- pyrrolizin-7a(5H)-yl) methoxy)- 5,6,6a,7,8,9,10,11- octahydro-4-oxa-3,11a,12,14,15- pentaaza-7,10- methanocyclohepta[4,5] cycloocta[1,2,3-de]naphthalen- 2-yl)-5-methyl-4- (trifluoromethyl)aniline | 6.69 (s, 1H), 6.42 (s, 1H), 5.32 (d, J = 53.6 Hz, 1H), 4.71-3.77 (m, 8H), 3.63 (d, J = 57.3 Hz, 2H), 3.49- 3.37 (m, 1H), 3.26- 3.05 (m, 3H), 2.54- 2.19 (m, 6H), 2.19- 1.54 (m, 7H). (3 H obscured by solvent peak) | 662.5 |
| 215 | 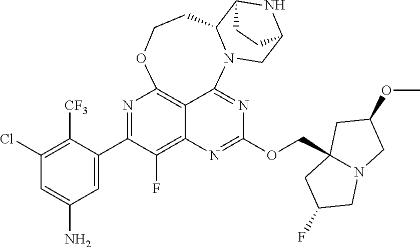 [SMILES: CO[C@H]1CN2C[C@H](F)C[C@]2(COc2nc3c4c(nc(-c5cc(N)cc(Cl)c5C(F)(F)F)c(F)c4n2)OCC[C@@H]2[C@@H]4CC[C@H](CN32)N4)C1] | 3-chloro-5-((6aR,7S,10R)-1- fluoro-13-(((2R,6R,7aS)-2- fluoro-6-methoxytetrahydro- 1H-pyrrolizin-7a(5H)-yl) methoxy)- 5,6,6a,7,8,9,10,11- octahydro-4-oxa-3,11a,12,14,15- pentaaza-7,10- methanocyclohepta[4,5] cycloocta[1,2,3-de]naphthalen- 2-yl)-4-(trifluoromethyl) aniline | 6.89 (d, J = 2.0 Hz, 1H), 6.52 (d, J = 25.0 Hz, 1H), 5.32 (d, J = 54.2 Hz, 1H), 4.65- 3.82 (m, 8H), 3.77- 3.51 (m, 2H), 3.48- 3.39 (m, 1H), 3.18- 3.14 (m, 3H), 2.49- 2.21 (m, 3H), 2.19- 1.62 (m, 7H). (3 H obscured by solvent peak) | 682.4 |
| 216 | 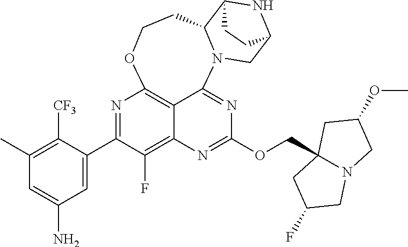 | 3-((6aR,7S,10R)-1-fluoro-13- (((2R,6S,7aS)-2-fluoro-6- methoxytetrahydro-1H- pyrrolizin-7a(5H)-yl)methoxy)- 5,6,6a,7,8,9,10,11- octahydro-4-oxa-3,11a,12,14,15- pentaaza-7,10- methanocyclohepta[4,5]cyclo- octa[1,2,3-de]naphthalen-2-yl)- 5-methyl-4-(trifluoromethyl) aniline | 6.68 (s, 1H), 6.47-6.42 (m, 1H), 5.41-5.24 (m, 1H), 4.39-3.90 (m, 8H), 3.65 (s, 1H), 3.60-3.47 (m, 1H), 3.42-3.39 (m, 1H), 3.34 (s, 3H), 3.28- 3.14 (m, 2H), 3.02-2.94 (m, 1H), 2.48-2.38 (m, 4H), 2.35-2.19 (m, 2H), 2.12-1.73 (m, 7H). | 662.6 |
| 217 | 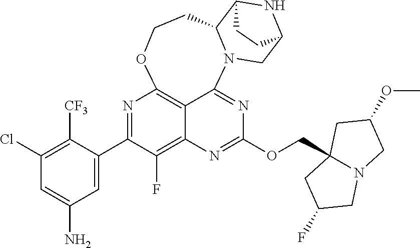 [SMILES: CO[C@@H]1CN2C[C@H](F)C[C@]2(COc2nc3c4c(nc(-c5cc(N)cc(Cl)c5C(F)(F)F)c(F)c4n2)OCC[C@@H]2[C@@H]4CC[C@H](CN32)N4)C1] | 3-chloro-5-((6aR,7S,10R)-1- fluoro-13-(((2R,6S,7aS)-2- fluoro-6-methoxytetahydro-1H- pyrrolizin-7a(5H)-yl)methoxy)- 5,6,6a,7,8,9,10,11- octahydro-4-oxa-3,11a,12,14,15- pentaaza-7,10- methanocyclohepta[4,5]cyclo- octa[1,2,3-de]naphthalen-2-yl)- 4-(trifluoromethyl)aniline | 6.99 (s, 1H), 6.63 (s, 1H), 5.64 (d, J = 51.8 Hz, 1H), 4.78-4.15 (m, 9H), 4.14-4.00 (m, 3H), 3.91 (t, J = 14.8 Hz, 1H), 3.5-3.44 (m, 4H), 2.83-2.67 (m, 3H), 2.55-2.24 (m, 4H), 2.16 (s, 3H). | 682.3 |

Examples 218-222: The following examples were prepared in a manner similar to that in Example 142 by replacing the corresponding substitution or coupling partners.

| | | | | MS |
|---|---|---|---|---|
| Ex- | | | 1H NMR (500 MHz, | (ESI, m/e) |
| ample | Structure | Compound Name | CD₃OD) δ | [M + H]⁺ |
| 218 | 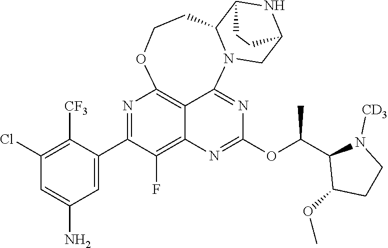 [SMILES: [2H]C([2H])([2H])N1CC[C@H](OC)[C@H]1[C@H](C)Oc1nc2c3c(nc(-c4cc(N)cc(Cl)c4C(F)(F)F)c(F)c3n1)OCC[C@@H]1[C@@H]3CC[C@H](CN21)N3] | 3-chloro-5-((6aR,7S,10R)-1- fluoro-13-((S)-1-((2R,3S)-3- methoxy-1-(methyl- d3)pyrrolidin-2-yl)ethoxy)- 5,6,6a,7,8,9,10,11- octahydro-4-oxa-3,11a,12,14,15- pentaaza-7,10- methanocyclohepta[4,5]cyclo- octa[1,2,3-de]naphthalen-2-yl)- 4-(trifluoromethyl)aniline | 6.88 (d, J = 2.2 Hz, 1H), 6.52 (d, J = 33.0 Hz, 1H), 5.35-5.29 (m, 1H), 4.45-4.21 (m, 3H), 4.12-3.9 (m, 2H), 3.89- 3.52 (m, 4H), 3.26- 3.21 (m, 1H), 3.02 (s, 1H), 2.76 (s, 1H), 2.57 (s, 1H), 2.18-1.75 (m, 9H), 1.44 (d, J = 6.3 Hz, 3H). | 655.6 |
| 219 | 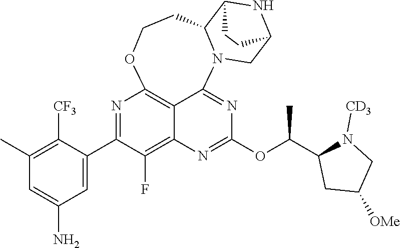 [SMILES: [2H]C([2H])([2H])N1C[C@H](OC)C[C@H]1[C@H](C)Oc1nc2c3c(nc(-c4cc(N)cc(C)c4C(F)(F)F)c(F)c3n1)OCC[C@@H]1[C@@H]3CC[C@H](CN21)N3] | 3-((6aR,7S,10R)-1-fluoro-13- ((S)-1-((2S,4R)-4-methoxy-1- (methyl-d3)pyrrolidin-2- yl)ethoxy)- 5,6,6a,7,8,9,10,11- octahydro-4-oxa-3,11a,12,14,15- pentaaza-7,10- methanocyclohepta[4,5] cycloocta[1,2,3-de]naphthalen- 2-yl)-5-methyl- 4-(trifluoromethyl) aniline | 6.68 (s, 1H), 6.44 (d, J = 27.6 Hz, 1H), 5.31- 5.25 (m, 1H), 4.45-4.20 (m, 3H), 4.15-3.80 (m, 3H), 3.72-3.52 (m, 2H), 3.42-3.31 (m, 4H), 3.00 (s, 1H), 2.55-2.35 (m, 4H), 2.20-1.69 (m, 8H), 1.39 (d, J = 6.1 Hz, 3H). | 635.6 |
| 220 | 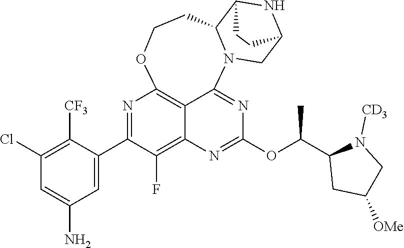 [SMILES: [2H]C([2H])([2H])N1C[C@H](OC)C[C@H]1[C@H](C)Oc1nc2c3c(nc(-c4cc(N)cc(Cl)c4C(F)(F)F)c(F)c3n1)OCC[C@@H]1[C@@H]3CC[C@H](CN21)N3] | 3-chloro-5-((6aR,7S,10R)-1- fluoro-13-((S)-1-((2S,4R)-4- methoxy-1-(methyl- d3)pyrrolidin-2-yl)ethoxy)- 5,6,6a,7,8,9,10,11- octahydro-4-oxa-3,11a,12,14,15- pentaaza-7,10- methanocyclohepta[4,5] cycloocta[1,2,3-de] naphthalen-2-yl)-4- (trifluoromethyl)aniline | 6.88 (d, J = 2.1 Hz, 1H), 6.51 (d, J = 36.4 Hz, 1H), 5.28 (p, J = 6.3 Hz, 1H), 4.32 (d, J = 44.3 Hz, 3H), 3.96 (dd, J = 47.8, 36.6 Hz, 3H), 3.74-3.48 (m, 2H), 3.38-3.27 (m, 4H), 2.99 (d, J = 7.3 Hz, 1H), 2.47 (d, J = 6.9 Hz, 1H), 2.23-1.71 (m, 8H), 1.39 (d, J = 6.3 Hz, 3H). | 655.5 |
| 221 | 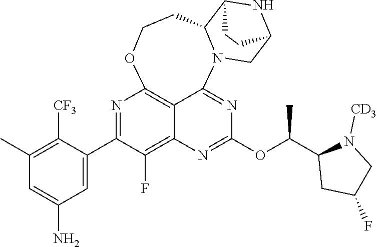 [SMILES: [2H]C([2H])([2H])N1C[C@H](F)C[C@H]1[C@H](C)Oc1nc2c3c(nc(-c4cc(N)cc(C)c4C(F)(F)F)c(F)c3n1)OCC[C@@H]1[C@@H]3CC[C@H](CN21)N3] | 3-((6aR,7S,10R)-1-fluoro-13- ((S)-1-((2S,4R)-4-fluoro-1- (methyl-d3)pyrrolidin-2- yl)ethoxy)- 5,6,6a,7,8,9,10,11- octahydro-4-oxa-3,11a,12,14,15- pentaaza-7,10- methanocyclohepta[4,5]cyclo- octa[1,2,3-de]naphthalen-2- yl)-5-methyl-4- (trifluoromethyl)aniline | 6.69 (s, 1H), 6.43 (s, 1H), 5.38-5.27 (m, 1H), 5.24-5.06 (m, 1H), 4.58 (s, 2H), 4.45- 4.0 (m, 4H), 3.95-3.7 (m, 3H), 2.80-2.74 (m, 1H), 2.42 (d, J = 1.9 Hz, 3H), 2.34-1.77 (m, 8H), 1.40 (d, J = 6.3 Hz, 3H). | 623.5 |
| 222 | 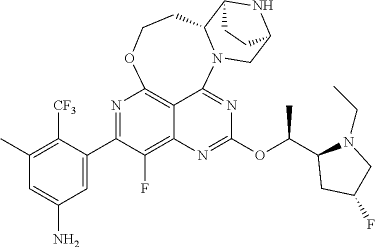 [SMILES: CCN1C[C@H](F)C[C@H]1[C@H](C)Oc1nc2c3c(nc(-c4cc(N)cc(C)c4C(F)(F)F)c(F)c3n1)OCC[C@@H]1[C@@H]3CC[C@H](CN21)N3] | 3-((6aR,7S,10R)-13-((S)-1- ((2S,4R)-1-ethyl-4- fluoropyrrolidin-2-yl)ethoxy)-1- fluoro- 5,6,6a,7,8,9,10,11- octahydro-4-oxa-3,11a,12,14,15- pentaaza-7,10-methanocyclo- hepta[4,5]cycloocta[1,2,3-de] naphthalen-2-yl)-5- methyl-4-(trifluoromethyl) aniline | 6.68 (s, 1H), 6.44 (d, J = 26.6 Hz, 1H), 5.38- 5.30 (m, 1H), 5.15 (d, J = 54.7 Hz, 1H), 4.50- 3.82 (m, 5H), 3.72-3.5 (m, 2H), 3.45-3.39 (m, 1H), 3.38-3.33 (m, 1H), 3.13-3.07 (m, 1H), 2.76 (dd, J = 28.5, 12.5 Hz, 1H), 2.68- 2.55 (m, 1H), 2.42 (d, J = 1.9 Hz, 3H), 2.27- 1.67 (m, 8H), 1.37 (d, J = 6.3 Hz 3H), 1.06 (t, J = 7.2 Hz, 3H). | 634.5 |

### Example 223: 3-((6aR,7S,10R)-1-fluoro-13-((S)-1-((1S,2S,5R)-3-methyl-3-azabicyclo[3.1.0]hexan-2-yl)ethoxy)-5,6,6a,7,8,9,10,11-octahydro-4-oxa-3,11a,12,14,15-pentaaza-7,10-methanocyclohepta[4,5]cycloocta[1,2,3-de]naphthalen-2-yl)-5-methyl-4-(trifluoromethyl)aniline

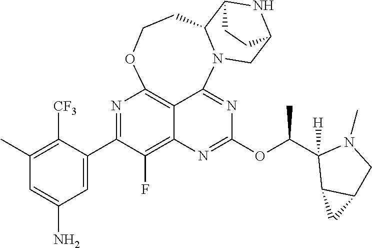 [SMILES: Cc1cc(N)cc(-c2nc3c4c(nc(O[C@@H](C)[C@@H]5[C@H]6C[C@H]6CN5C)nc4c2F)N2C[C@H]4CC[C@H](N4)[C@H]2CCO3)c1C(F)(F)F]

### Synthetic Route

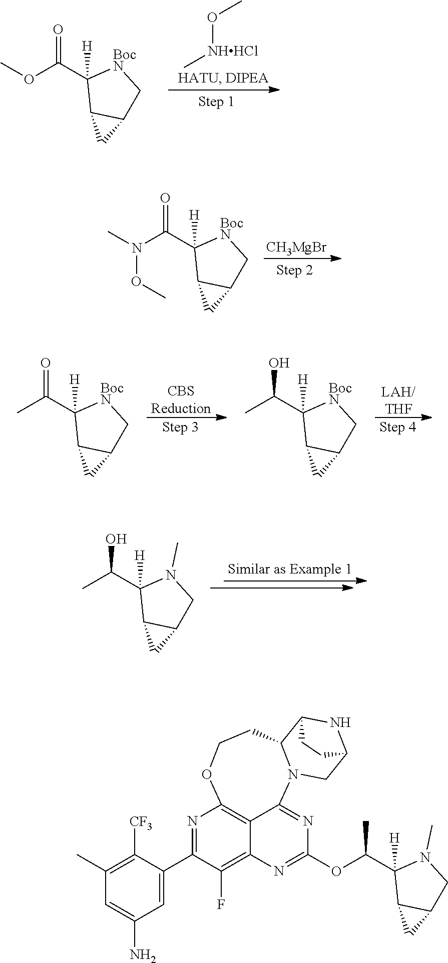

### Step 1: tert-butyl (1S,2S,5R)-2-(methoxy(methyl)carbamoyl)-3-azabicyclo[3.1.0]hexane-3-carboxylate

To a solution of (1S,2S,5R)-3-(tert-butoxycarbonyl)-3-azabicyclo[3.1.0]hexane-2-carboxylic acid (2.0 g, 8.81 mmol), N,O-dimethylhydroxylamine hydrochloride (1.1 g, 11.4 mmol) and DIPEA (2.8 g, 22.0 mmol) in DCM (20 mL) was added HATU (4.4 g, 11.5 mmol) at rt. The resulting mixture was stirred for 2 hours, and then concentrated in vacuo. The residue was purified by column chromatography (eluting with PE/EA=3/1) to obtain the title product as a light yellow solid (1.8 g, 76% yield). MS (ESI, m/e) [M+23]⁺ 293.5.

### Step 2: tert-butyl (1S,2S,5R)-2-acetyl-3-azabicyclo[3.1.0]hexane-3-carboxylate

To a solution of tert-butyl (1S,2S,5R)-2-(methoxy(methyl)carbamoyl)-3-azabicyclo[3.1.0]hexane-3-carboxylate (1.8 g, 6.67 mmol) in THF was added CH₃MgBr (6.7 mL, 20.0 mmol) at 0° C. and stirred for 2 h. The reaction mixture was concentrated in vacuo. The residue was purified by column chromatography (eluting with PE/EA=5/1) to obtain the title product as a colorless oil (1.2 g, 79% yield). MS (ESI, m/e) [M+23]⁺ 248.5.

### Step 3: tert-butyl (1S,2S,5R)-2-((R)-1-hydroxyethyl)-3-azabicyclo[3.1.0]hexane-3-carboxylate

To a solution of (R)-methyl oxazaborolidine (170 mg, 0.53 mmol) in THF (10 mL) was added B₂H₆ (3.43 mL, 3.43 mmol, 1 M THF solution) at −10° C. The resulting mixture was stirred for 1 hour, followed by addition of tert-butyl (1S,2S,5R)-2-acetyl-3-azabicyclo[3.1.0]hexane-3-carboxylate (600 mg, 2.64 mmol). The resulting mixture was stirred at −10° C. for 1 hour. The reaction was then quenched by addition of MeOH. The crude was concentrated in vacuo, and purified by column chromatography (eluting with DCM/MeOH=20/1) to obtain the title compound as a colorless oil (600 mg, 99% yield). MS (ESI, m/e) [M+23]⁺ 250.5.

### Step 4: (R)-1-((1S,2S,5R)-3-methyl-3-azabicyclo[3.1.0]hexan-2-yl)ethan-1-ol

To a solution of tert-butyl (1S,2S,5R)-2-((R)-1-hydroxyethyl)-3-azabicyclo[3.1.0]hexane-3-carboxylate (600 mg, 2.64 mmol) in THF (10 mL) was added LiAlH₄ (200 mg, 5.28 mmol) at rt. The resulting mixture was heated to reflux and stirred for 5 hours. The reaction mixture was cooled to rt, followed by addition of Na₂SO₄·10H₂O. The resulting mixture was filtrated. The filtrate was concentrated in vacuo to give the title compound as a colorless oil (280 mg, 75% yield), which was used directly in the next step without further purification. MS (ESI, m/e) [M+1]+ 142.5.

Steps 5-7: Example 223 was prepared in similar fashion as Example 01 by replacing (2,2-difluorotetrahydro-1H-pyrrolizin-7a(5H)-yl)methanol with (R)-1-((1S,2S,5R)-3-methyl-3-azabicyclo[3.1.0]hexan-2-yl)ethan-1-ol. The title product was purified by prep-HPLC (Column: Xselect C18, 19*150 mm, 5 μm; Mobile Phase A: H₂O (0.1% FA), Mobile Phase B: MeCN (0.1% FA); Flow Rate: 17 mL/min; Room Temperature; Detector: UV 214 & 254 inn; Gradient: 13% to 23% B in 11 min, 23% to 90% B in 0.2 min, 90% B hold in 2 min, 90% to 13% B in 0.3 min, 13% B hold in 1.5 min ¹H NMR (500 MHz, CD₃OD) 6.68 (s, 1H), 6.44-6.40 (m, 1H), 5.40-5.37 (m, 1H), 4.35-4.08 (m, 4H), 3.72-3.63 (m, 2H), 3.09-3.08 (m, 1H), 2.64-2.62 (m, 1H), 2.49-2.42 (m, 6H), 2.10-1.86 (m, 5H), 1.61-1.29 (m, 7H), 0.90-0.77 (m, 2H), 0.31-0.27 (m, 1H). MS (ESI, m/e) [M+H]⁺ 614.5.

Examples 224-225: The following examples were prepared in a manner similar to that in Example 223.

| | | | | MS |
|---|---|---|---|---|
| | | | 1H NMR (500 MHz, | (ESI, m/e) |
| Example | Structure | Compound Name | CD₃OD) δ | [M + H]⁺ |
| 224 | 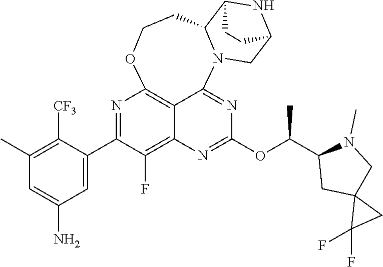 | 3-((6aR,7S,10R)-13-((1S)-1- ((6S)-1,1-difluoro-5-methyl-5- azaspiro[2.4]heptan-6- yl)ethoxy)-1-fluoro- 5,6,6a,7,8,9,10,11- octahydro-4-oxa-3,11a,12,14,15- pentaaza[7,10]methanocyclo- hepta[4,5]cycloocta[1,2,3- de]naphthalen-2-yl)-5-methyl- 4-(trifluoromethyl)aniline | 6.68 (s, 1H), 6.47-6.41 (m, 1H), 5.41-5.39 (m, 1H), 4.36-3.57 (m, 9H), 2.95-2.70 (m, 3H), 2.49-2.42 (m, 6H), 2.30-1.72 (m, 9H), 1.39- 1.29 (m, 6H). | 664.5 |
| | (isomer 1) | | | |
| 225 | 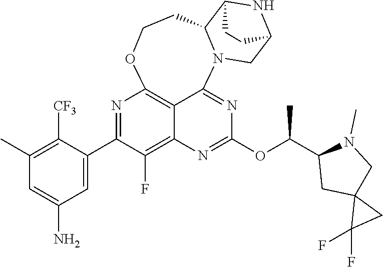 | 3-((6aR,7S,10R)-13-((1S)-1- ((6S)-1,1-difluoro-5-methyl-5- azaspiro[2.4]heptan-6- yl)ethoxy)-1-fluoro- 5,6,6a,7,8,9,10,11- octahydro-4-oxa-3,11a,12,14,15- pentaaza[7,10]methanocyclo- hepta[4,5]cycloocta[1,2,3- de]naphthalen-2-yl)-5-methyl-4- (trifluoromethyl)aniline | 6.69 (s, 1H), 6.42-6.41 (m, 1H), 5.41-5.37 (m, 1H), 4.36-3.57 (m, 9H), 2.95-2.70 (m, 3H), 2.49-2.42 (m, 6H), 2.31-1.72 (m, 9H), 1.39-1.29 (m, 6H). | 664.5 |
| | (isomer 2) | | | |

### Example 226: 3-((6aR,7S,10R)-1-fluoro-13-((S)-1-((S)-1-(tetrahydro-2H-pyran-4-yl)pyrrolidin-2-yl)ethoxy)-5,6,6a,7,8,9,10,11-octahydro-4-oxa-3,11a,12,14,15-pentaaza-7,10-methanocyclohepta[4,5]cycloocta[1,2,3-de]naphthalen-2-yl)-5-methyl-4-(trifluoromethyl)aniline

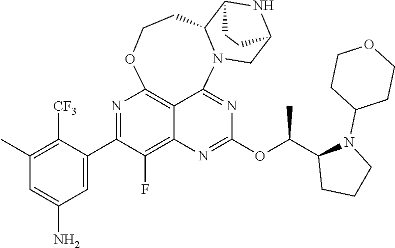 [SMILES: Cc1cc(N)cc(-c2nc3c4c(nc(O[C@@H](C)[C@@H]5CCCN5C5CCOCC5)nc4c2F)N2C[C@H]4CC[C@H](N4)[C@H]2CCO3)c1C(F)(F)F]

### Synthetic Route

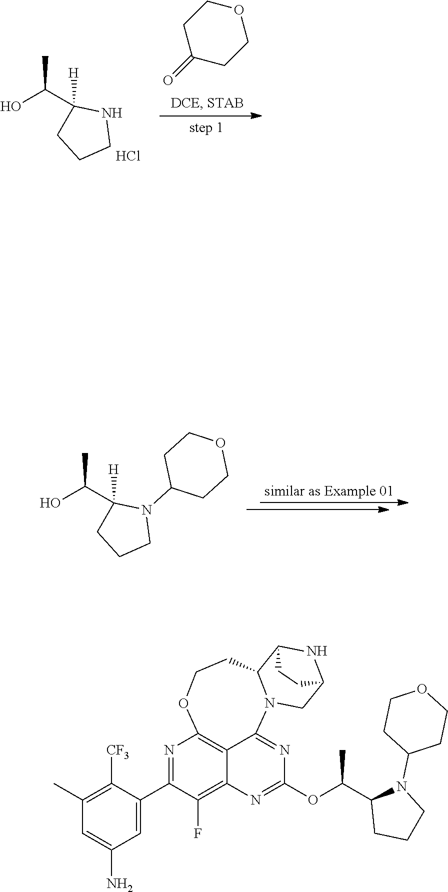

### Step 1: (S)-1-((S)-1-(tetrahydro-2H-pyran-4-yl)pyrrolidin-2-yl)ethan-1-ol

To a solution of (S)-1-((S)-pyrrolidin-2-yl)ethan-1-ol hydrochloride (100 mg, 0.662 mmol) in DCE (3 mL) was added tetrahydro-4H-pyran-4-one (199 mg, 1.99 mmol) at room temperature. The resulting mixture was stirred at room temperature for 30 min. To the above mixture was added STAB (421 mg, 1.99 mmol) at room temperature. The resulting mixture was stirred at room temperature for 16 h. The mixture was quenched with saturated Na₂CO₃ solution (3 mL) at 0° C. and extracted with DCM (5 mL×3). The combined extracts were dried over Na₂SO₄ and concentrated in vacuo. The resulting residue was purified by silica gel column chromatography (DCM/MeOH=10:1) to give the title product (86 mg, 65%). MS (ESI, m/e) [M+1]⁺ 200.2.

Steps 2-4: Example 226 was prepared in similar fashion as Example 01 by replacing (2,2-difluorotetrahydro-1H-pyrrolizin-7a(5H)-yl)methanol with abovementioned (S)-1-((S)-1-(tetrahydro-2H-pyran-4-yl)pyrrolidin-2-yl)ethan-1-ol. ¹H NMR (500 MHz, CD₃OD) δ 6.68 (s, 1H), 6.43 (d, J=29.5 Hz, 1H), 5.32-5.26 (m, 1H), 4.44-4.17 (m, 3H), 4.13-3.85 (m, 4H), 3.68 (s, 1H), 3.63-3.51 (m, 1H), 3.44-3.30 (m, 3H), 2.95 (s, 2H), 2.65 (s, 1H), 2.42 (d, J=1.9 Hz, 3H), 2.15-1.95 (m, 3H), 1.92-1.70 (m, 9H), 1.68-1.52 (m, 2H), 1.38 (d, J=6.1 Hz, 3H). MS (ESI, m/e) [M+1]⁺ 672.5.

Examples 227-230: The following examples were prepared in a manner similar to that in Example 226 by by replacing the corresponding substitution or coupling partners.

| Ex- | | | | MS |
|---|---|---|---|---|
| am- | | | 1H NMR (500 MHz, | (ESI, m/e) |
| ple | Structure | Compound Name | CD₃OD) δ | [M + H]⁺ |
| 227 | 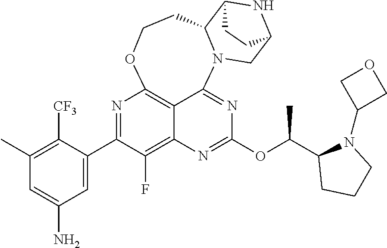 [SMILES: Cc1cc(N)cc(-c2nc3c4c(nc(O[C@@H](C)[C@@H]5CCCN5C5COC5)nc4c2F)N2C[C@H]4CC[C@H](N4)[C@H]2CCO3)c1C(F)(F)F] | 3-((6aR,7S,10R)-1-fluoro-13- ((S)-1-((S)-1-(oxetan-3- yl)pyrrolidin-2-yl)ethoxy)- 5,6,6a,7,8,9,10,11-octahydro- 4-oxa-3,11a,12,14,15- pentaaza-7,10-methanocyclo- hepta[4,5]cycloocta[1,2,3-de] naphthalen-2-yl)-5-methyl-4- (trifluoromethyl)aniline | 6.68 (s, 1H), 6.44 (d, J = 28.9 Hz, 1H), 5.19- 5.13 (m, 1H), 4.75-4.55 (m, 4H), 4.44-4.2 (m, 3H), 4.19-3.83 (m, 3H), 3.70 (s, 1H), 3.66- 3.49 (m, 1H), 3.05- 3.98 (m, 1H), 2.94- 2.84 (m, 1H), 2.59-2.53 (m, 1H), 2.42 (d, J = 1.9 Hz, 3H), 2.18-1.92 (m, 4H), 1.92-1.71 (m, 6H), 1.36 (d, J = 6.2 Hz, 3H). | 644.5 |
| 228 | 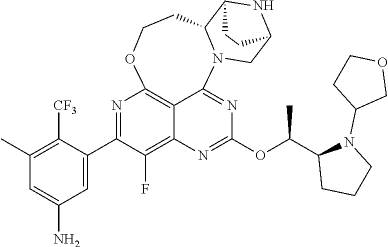 [SMILES: Cc1cc(N)cc(-c2nc3c4c(nc(O[C@@H](C)[C@@H]5CCCN5C5CCOC5)nc4c2F)N2C[C@H]4CC[C@H](N4)[C@H]2CCO3)c1C(F)(F)F] | 3-((6aR,7S,10R)-1-fluoro-13- ((1S)-1-((2S)-1-(tetrahydro- furan-3-yl)pyrrolidin-2-yl) ethoxy)- 5,6,6a,7,8,9,10,11-octahydro- 4-oxa-3,11a,12,14,15- pentaaza-7,10-methanocyclo- hepta[4,5]cycloocta[1,2,3-de] naphthalen-2-yl)-5-methyl- 4-(trifluoromethyl)aniline | 6.68 (s, 1H), 6.44 (d, J = 27.6 Hz, 1H), 5.33- 5.27 (m, 1H), 4.42-4.2 (m, 3H), 4.16-3.85 (m, 3H), 3.79-3.68 (m, 4H), 3.65-3.5 (m, 2H), 3.19-3.15 (m, 1H), 2.97 (s, 1H), 2.60 (s, 1H), 2.42 (d, J = 1.9 Hz, 3H), 2.23-1.96 (m, 5H), 1.95-1.7 (m, 7H), 1.37 (d, J = 6.3 Hz, 3H). | 658.6 |
| | (isomer 1) | | | |
| 229 | 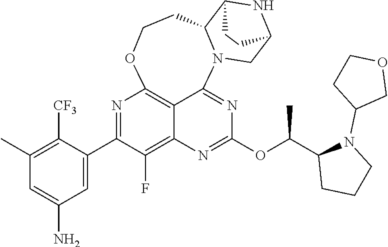 [SMILES: Cc1cc(N)cc(-c2nc3c4c(nc(O[C@@H](C)[C@@H]5CCCN5C5CCOC5)nc4c2F)N2C[C@H]4CC[C@H](N4)[C@H]2CCO3)c1C(F)(F)F] | 3-((6aR,7S,10R)-1-fluoro-13- ((1S)-1-((2S)-1-(tetrahydro- furan-3-yl)pyrrolidin-2-yl) ethoxy)- 5,6,6a,7,8,9,10,11-octahydro- 4-oxa-3,11a,12,14,15- pentaaza-7,10- methanocyclohepta[4,5] cycloocta[1,2,3-de] naphthalen-2-yl)-5-methyl-4- (trifluoromethyl)aniline | 6.68 (s, 1H), 6.43 (d, J = 27.9 Hz, 1H), 5.28- 5.22 (m, 1H), 4.45-4.15 (m, 3H), 4.05-3.95 (m, 2H), 3.94-3.82 (m, 2H), 3.75-3.65 (m, 4H), 3.64-3.56 (m, 1H), 3.11 (s, 1H), 3.00 (s, 1H), 2.65-2.55 (m, 1H), 2.42 (d, J = 1.9 Hz, 3H), 2.21-1.97 (m, 4H), 1.94-1.74 (m, 8H), 1.36 (d, J = 6.3 Hz, 3H). | 658.6 |
| | (isomer 2) | | | |
| 230 | 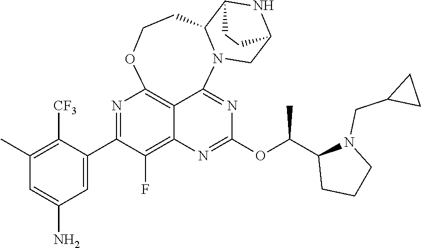 [SMILES: Cc1cc(N)cc(-c2nc3c4c(nc(O[C@@H](C)[C@@H]5CCCN5CC5CC5)nc4c2F)N2C[C@H]4CC[C@H](N4)[C@H]2CCO3)c1C(F)(F)F] | 3-((6aR,7S,10R)-13-((S)-1- ((S)-1-(cyclopropylmethyl) pyrrolidin-2-yl)ethoxy)-1- fluoro- 5,6,6a,7,8,9,10,11-octahydro- 4-oxa-3,11a,12,14,15- pentaaza-7,10-methano- cyclohepta[4,5]cycloocta [1,2,3-de]naphthalen-2-yl)- 5-methyl-4-(trifluoromethyl) aniline | 6.69 (s, 1H), 6.44 (d, J = 34.3 Hz, 1H), 5.35- 5.24 (m, 1H), 4.42-3.79 (m, 5H), 3.7-3.35 (m, 5H), 3.16-2.9 (m, 1H), 2.6-2.3 (m, 4H), 2.20- 1.66 (m, 10H), 1.41 (d, J = 5.5 Hz, 3H), 0.95 (s, 1H), 0.60-0.40 (m, 2H), 0.30-0.10 (m, 2H). | 642.6 |

### Example 231: 3-((6aR,7S,10R)-13-((S)-1-((S)-1-allylpyrrolidin-2-yl) ethoxy)-1-fluoro-5,6,6a,7,8,9,10,11-octahydro-4-oxa-3,11a,12,14,15-pentaaza-7,10-methanocyclohepta[4,5]cycloocta[1,2,3-de]naphthalen-2-yl)-5-methyl-4-(trifluoromethyl)aniline

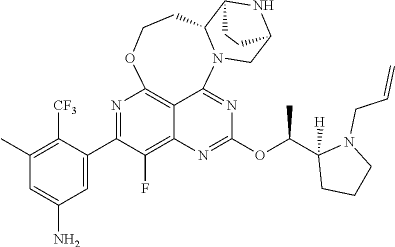 [SMILES: C=CCN1CCC[C@H]1[C@H](C)Oc1nc2c3c(nc(-c4cc(N)cc(C)c4C(F)(F)F)c(F)c3n1)OCC[C@@H]1[C@@H]3CC[C@H](CN21)N3]

### Synthetic Route

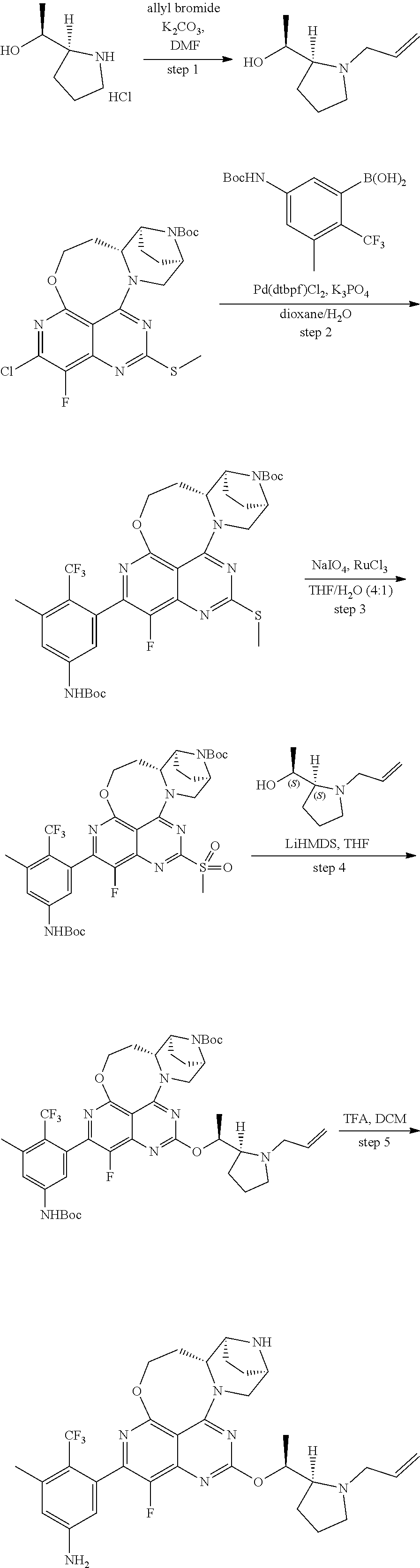

### Step 1: (S)-1-((S)-1-allylpyrrolidin-2-yl) ethan-1-ol

To a solution of (S)-1-((S)-pyrrolidin-2-yl)ethan-1-ol hydrochloride (100 mg, 0.66 mmol) and 3-bromoprop-1-ene (79 mg, 0.66 mmol) in DMF (2 mL) was added K₂CO₃ (274 mg, 1.99 mmol). The mixture was stirred at room temperature for 2 hours. The reaction mixture was then diluted with ethyl acetate (20 mL) and washed with H₂O (3×20 mL) and brine (20 mL). The organic layer was dried over Na₂SO₄, and concentrate in vacuo. The crude was purified by silica gel column chromatography (CH₂Cl₂/MeOH=9:1) to afford the title product (70 mg, 68% yield) as a white oil. MS (ESI, m/e) [M+1]+156.

### Step 2: tert-butyl (6aR,7S,10R)-2-(5-((tert-butoxycarbonyl) amino)-3-methyl-2-(trifluoromethyl) phenyl)-1-fluoro-13-(methylthio)-5,6,6a,7,8,9,10,11-octahydro-4-oxa-3,11a,12,14,15-pentaaza-7,10-methanocyclohepta [4,5]cycloocta[1,2,3-de]naphthalene-15-carboxylate

To a solution of tert-butyl (6aR,7S,10R)-2-chloro-1-fluoro-13-(methylthio)-5,6,6a,7,8,9,10,11-octahydro-4-oxa-3,11a,12,14,15-pentaaza-7,10-methanocyclohepta[4,5]cycloocta[1,2,3-de]naphthalene-15-carboxylate (120 mg, 0.25 mmol) and (5-((tert-butoxycarbonyl)amino)-3-methyl-2-(trifluoromethyl)phenyl)boronic acid (159 mg, 0.5 mmol) in dioxane (2 mL) and H₂O (0.4 mL) was added Pd(dtbpf)Cl₂ (16 mg, 0.025 mmol) and K₃PO₄ (159 mg, 0.75 mmol) at 25° C. under a nitrogen atmosphere. The resulting mixture was stirred at 90° C. for 2 hours. The mixture was then diluted with ethyl acetate (20 mL) and washed with H₂O (20 mL) and brine (20 mL). The organic layer was dried over Na₂SO₄, and concentrate in vacuo. The crude was purified by silica gel column chromatography (DCM/MeOH=9:1) to afford the title product (90 mg, 50% yield) as a yellow solid. MS (ESI, m/e) [M+1]⁺ 721.

### Step 3: tert-butyl (6aR,7S,10R)-2-(5-((tert-butoxycarbonyl)amino)-3-methyl-2-(trifluoromethyl)phenyl)-1-fluoro-13-(methylsulfonyl)-5,6,6a,7,8,9,10,11-octahydro-4-oxa-3,11a,12,14,15-pentaaza-7,10-methanocyclohepta[4,5]cycloocta[1,2,3-de]naphthalene-15-carboxylate

To a solution of tert-butyl (6aR,7S,10R)-2-(5-((tert-butoxycarbonyl)amino)-3-methyl-2-(trifluoromethyl)phenyl)-1-fluoro-13-(methylthio)-5,6,6a,7,8,9,10,11-octahydro-4-oxa-3,11a,12,14,15-pentaaza-7,10-methanocyclohepta[4,5]cycloocta[1,2,3-de]naphthalene-15-carboxylate (90 mg, 0.125 mmol) in THF (2 mL) and H₂O (0.5 mL) was added RuCl₃ (2.58 mg, 0.0125 mmol) and NaIO₄ (81 mg, 0.375 mmol) at room temperature under a nitrogen atmosphere. The resulting mixture was stirred at room temperature for 1 hour. The mixture was diluted with ethyl acetate (20 mL) and washed with H₂O (20 mL) and brine (20 mL). The organic layer was dried over Na₂SO₄, and concentrate in vacuo. The crude was purified by prep-TLC (DCM/MeOH=10:1) to afford the title product (65 mg, 69% yield) as a light yellow solid. MS (ESI, m/e) [M+1]+ 753.

### Step 4: tert-butyl (6aR,7S,10R)-13-((S)-1-((S)-1-allylpyrrolidin-2-yl)ethoxy)-2-(5-((tert-butoxycarbonyl)amino)-3-methyl-2-(trifluoromethyl)phenyl)-1-fluoro-5,6,6a,7,8,9,10,11-octahydro-4-oxa-3,11a,12,14,15-pentaaza-7,10-methanocyclohepta[4,5]cycloocta[1,2,3-de]naphthalene-15-carboxylate

To a solution of (S)-1-((S)-1-allylpyrrolidin-2-yl)ethan-1-ol (14.4 mg, 0.09 mmol) and 4A molecular sieves (50 mg) in THF (2 mL) was added dropwise LiHMDS in THF (0.09 mL, 1M, 0.09 mmol) at room temperature under a nitrogen atmosphere. The resulting mixture was stirred at room temperature for 0.5 hours and then added tert-butyl (6aR,7S,10R)-2-(5-((tert-butoxycarbonyl)amino)-3-methyl-2-(trifluoromethyl)phenyl)-1-fluoro-13-(methylsulfonyl)-5,6,6a,7,8,9,10,11-octahydro-4-oxa-3,11a,12,14,15-pentaaza-7,10-methanocyclohepta[4,5]cycloocta[1,2,3-de]naphthalene-15-carboxylate (35 mg, 0.047 mmol). The resulting mixture was stirred at room temperature for 1 hour. The mixture was diluted with ethyl acetate (10 mL) and washed with with H₂O (20 mL) and brine (20 mL). The organic layer was dried over Na₂SO₄, and concentrate in vacuo. The crude was purified by prep-TLC (DCM/MeOH=10:1) to afford the title product (5 mg, 13% yield) as a light yellow solid. MS (ESI, m/e) [M+1]+ 828.

### Step 4: 3-((6aR,7S,10R)-13-((S)-1-((S)-1-allylpyrrolidin-2-yl)ethoxy)-1-fluoro-5,6,6a,7,8,9,10,11-octahydro-4-oxa-3,11a,12,14,15-pentaaza-7,10-methanocyclohepta[4,5]cycloocta[1,2,3-de]naphthalen-2-yl)-5-methyl-4-(trifluoromethyl)aniline

To a solution of tert-butyl (6aR,7S,10R)-13-((S)-1-((S)-1-allylpyrrolidin-2-yl)ethoxy)-2-(5-((tert-butoxycarbonyl)amino)-3-methyl-2-(trifluoromethyl)phenyl)-1-fluoro-5,6,6a,7,8,9,10,11-octahydro-4-oxa-3,11a,12,14,15-pentaaza-7,10-methanocyclohepta[4,5]cycloocta[1,2,3-de]naphthalene-15-carboxylate (5 mg, 0.006 mmol) in DCM (1 mL) was added TFA (0.2 mL) at room temperature. The mixture was stirred at room temperature for 1 hour. The resulting mixture was concentrate in vacuo. The crude was purified by prep-HPLC (Column: Waters SunFire C18, 19*150 mm, 5 μm; Mobile Phase A: H₂O (0.1% FA), Mobile Phase B: MeCN (0.1% FA); Flow Rate: 17 mL/min; Room Temperature; Detector: UV 214 & 254 nm; Gradient: 12% to 28% B in 11 min, 28% to 90% B in 0.2 min, 90% B hold in 2 min, 90% to 12% B in 0.3 min, 12% B hold in 1.5 min) to afford the title product (0.42 mg, 11% yield) as a white solid. MS (ESI, m/e) [M+1]⁺ 628. 1H NMR (500 MHz, MeOD) δ 6.69 (s, 1H), 6.44 (d, J=24.4 Hz, 1H), 5.95-5.88 (m, 1H), 5.42-5.22 (m, 3H), 4.58 (s, 1H), 3.6-4.4 (m, 8H), 3.34 (s, 2H), 3.24-3.18 (m, 1H), 2.42 (d, J=1.9 Hz, 3H), 2.25-1.75 (m, 10H), 1.44 (d, J=6.2 Hz, 3H).

The following Examples 232-235 were prepared in similar fashion as Example 126-129 by replacing (5-methoxy-1,3-dimethylpiperidin-3-yl)methanol with the corresponding alkyl alcohol as a mixture of isomers, followed by further separation via chiral Prep-HPLC (Column: i-Cellulose-5, 21.2 mm×250 mm, 5 μm; Mobile Phase A: n-Hexane, Mobile Phase B: EtOH (with 0.2% 2M NH₃ in MeOH); Flow Rate: 18 mL/min; Temperature: 25° C.; Gradient: 50% B in 18 min).

| Ex- | | | ¹H NMR | MS |
|---|---|---|---|---|
| am- | | | (500 MHz, | (ESI, m/e) |
| ple | Structure | Compound Name | DMSO-d8) δ | [M + H]⁺ |
| 232 | 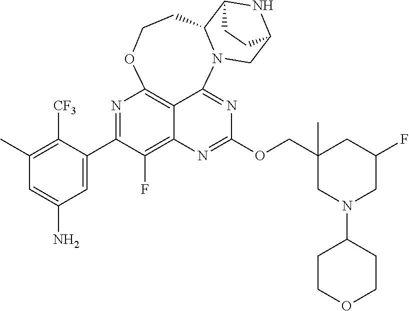 [SMILES: Cc1cc(N)cc(-c2nc3c4c(nc(OCC5(C)CC(F)CN(C6CCOCC6)C5)nc4c2F)N2C[C@H]4CC[C@H](N4)[C@H]2CCO3)c1C(F)(F)F] | 3-((6aR,7S,10R)-1-fluoro-13-((5- fluoro-3-methyl-1-(tetrahydro-2H- pyran-4-yl)piperidin-3-yl)methoxy)- 5,6,6a,7,8,9,10,11-octahydro- 4-oxa-3,11a,12,14,15- pentaaza-7,10-methanocyclohepta [4,5]cycloocta[1,2,3-de]naphthalen- 2-yl)-5-methyl-4-(trifluoromethyl) aniline | 6.59 (s, 1H), 6.36- 6.35 (m, 1H), 5.86 (s, 2H), 4.82-4.72 (m, 1H), 4.41-3.42 (m, 11H), 3.24-2.57 (m, 6H), 2.36-1.54 (m, 17H), 1.03 (s, 3H). | 704.5 |
| | (isomer 1) | | | |
| 233 | 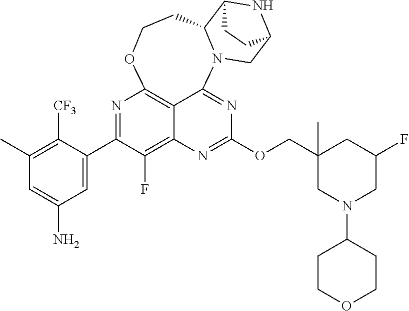 [SMILES: Cc1cc(N)cc(-c2nc3c4c(nc(OCC5(C)CC(F)CN(C6CCOCC6)C5)nc4c2F)N2C[C@H]4CC[C@H](N4)[C@H]2CCO3)c1C(F)(F)F] | 3-((6aR,7S,10R)-1-fluoro-13-((5- fluoro-3-methyl-1-(tetrahydro-2H- pyran-4-yl)piperidin-3-yl)methoxy)- 5,6,6a,7,8,9,10,11-octahydro- 4-oxa-3,11a,12,14,15- pentaaza-7,10-methanocyclohepta [4,5]cycloocta[1,2,3-de]naphthalen- 2-yl)-5-methyl-4-(trifluoromethyl) aniline | 6.59 (s, 1H), 6.36- 6.35 (m, 1H), 5.86 (s, 2H), 4.82-4.72 (m, 1H), 4.21-3.42 (m, 11H), 3.25-2.56 (m, 6H), 2.35-1.55 (m, 17H), 1.03 (s, 3H). | 704.5 |
| | (isomer 2) | | | |
| 234 | 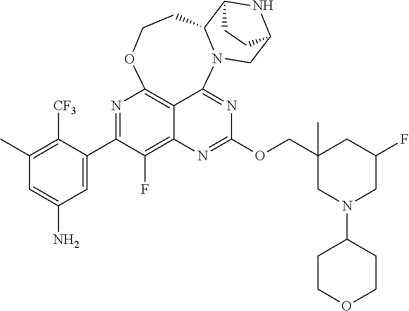 [SMILES: Cc1cc(N)cc(-c2nc3c4c(nc(OCC5(C)CC(F)CN(C6CCOCC6)C5)nc4c2F)N2C[C@H]4CC[C@H](N4)[C@H]2CCO3)c1C(F)(F)F] | 3-((6aR,7S,10R)-1-fluoro-13-((5- fluoro-3-methyl-1-(tetrahydro-2H- pyran-4-yl)piperidin-3-yl)methoxy)- 5,6,6a,7,8,9,10,11-octahydro- 4-oxa-3,11a,12,14,15- pentaaza-7,10-methanocyclohepta [4,5]cycloocta[1,2,3-de]naphthalen- 2-yl)-5-methyl-4-(trifluoromethyl) aniline | 6.59 (s, 1H), 6.36- 6.35 (m, 1H), 5.87 (s, 2H), 4.83-4.71 (m, 1H), 4.29-3.42 (m, 11H), 3.26-2.61 (m, 6H), 2.36-1.54 (m, 17H), 1.07 (s, 3H). | 704.5 |
| | (isomer 3) | | | |
| 235 | 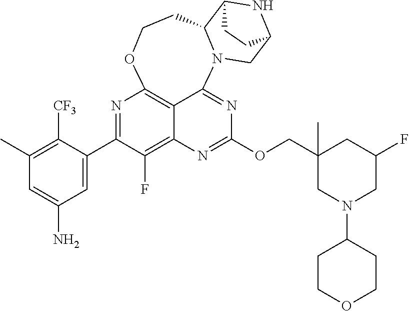 [SMILES: Cc1cc(N)cc(-c2nc3c4c(nc(OCC5(C)CC(F)CN(C6CCOCC6)C5)nc4c2F)N2C[C@H]4CC[C@H](N4)[C@H]2CCO3)c1C(F)(F)F] | 3-((6aR,7S,10R)-1-fluoro-13-((5- fluoro-3-methyl-1-(tetrahydro-2H- pyran-4-yl)piperidin-3-yl)methoxy)- 5,6,6a,7,8,9,10,11-octahydro- 4-oxa-3,11a,12,14,15- pentaaza-7,10-methanocyclohepta [4,5]cycloocta[1,2,3-de]naphthalen- 2-yl)-5-methyl-4-(trifluoromethyl) aniline | 6.59 (s, 1H), 6.37- 6.35 (m, 1H), 5.87 (s, 2H), 4.81-4.71 (m, 1H), 4.33-3.43 (m, 11H), 3.24-2.63 (m, 6H), 2.36-1.44 (m, 17H), 1.07 (s, 3H). | 704.5 |
| | (isomer 4) | | | |

### Examples 236 and 237: 3-((6aR,7S,10R)-1-fluoro-13-(((E)-4-(fluoromethylene)-1,3-dimethylpiperidin-3-yl)methoxy)-5,6,6a,7,8,9,10,11-octahydro-4-oxa-3,11a,12,14,15-pentaaza-7,10-methanocyclohepta[4,5]cycloocta[1,2,3-de]naphthalen-2-yl)-5-methyl-4-(trifluoromethyl)aniline

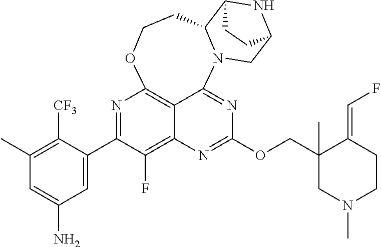 [SMILES: Cc1cc(N)cc(-c2nc3c4c(nc(OCC5(C)CN(C)CC/C5=C\F)nc4c2F)N2C[C@H]4CC[C@H](N4)[C@H]2CCO3)c1C(F)(F)F]

### Synthetic Route

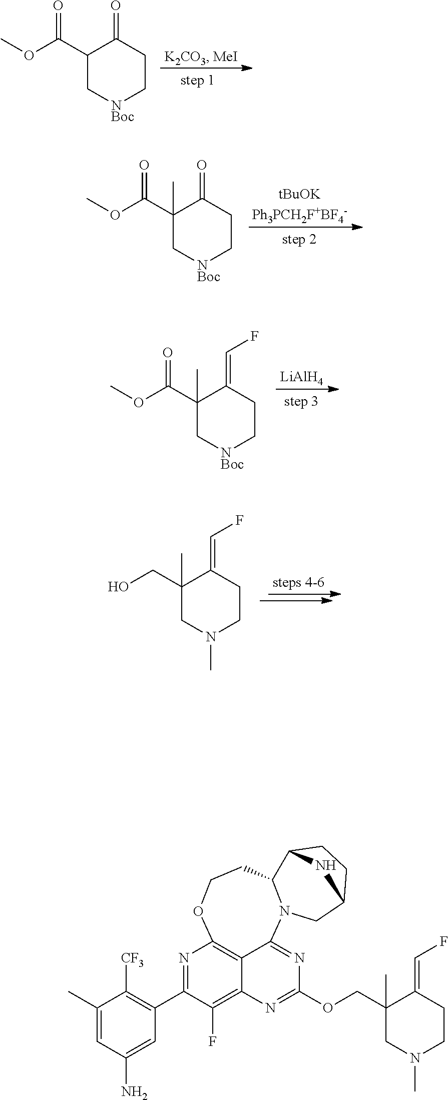

### Step 1: 1-(tert-butyl) 3-methyl 3-methyl-4-oxopiperidine-1,3-dicarboxylate

To a mixture of 1-(tert-butyl) 3-methyl 3-methyl-4-oxopiperidine-1,3-dicarboxylate (25.7 g, 100.0 mmol), K₂CO₃ (41.37 g, 300 mmol) in acetonitrile (500 mL) was added iodomethane (56.7 g, 400.0 mmol) at room temperature. The resulting mixture was stirred at room temperature for 16 hours. The reaction mixture was quenched with saturated NH₄Cl (200 mL), extracted with DCM (150 mL×3). The organic layer was dried over anhydrous Na₂SO₄, filtered, and concentrated. The crude was purified by silica gel column chromatography, eluted with 0-30% EA in PE to give the title product (24.4 g, 90 mmol). MS (ESI, m/e) [M+1]⁺ 272.1.

### Step 2: 1-(tert-butyl) 3-methyl (E)-4-(fluoromethylene)-3-methylpiperidine-1,3-dicarboxylate

To the solution of (fluoromethyl)triphenylphosphonium tetrafluoroborate (10.56 g, 27.64 mmol) in THF (100 mL) was added t-BuOK (1 M, 27.64 mL) at −78° C. The resulting mixture was stirred at −78° C. for 1 hrs, followed by addition of 1-(tert-butyl) 3-methyl 3-methyl-4-oxopiperidine-1,3-dicarboxylate (5.0 g, 18.4 mmol). The reaction mixture was stirred for 16 h at RT, and then quenched with saturated NH₄Cl (200 mL), and extracted with DCM (150 mL×3). The organic layer was dried over anhydrous Na₂SO₄, filtered, and concentrated. The crude was purified by silica gel column chromatography, eluted with 0-30% EA in PE to give the title product (3.2 g). MS (ESI, m/e) [M+1]⁺ 288.1.

### Step 3: (E)-(4-(fluoromethylene)-1,3-dimethylpiperidin-3-yl)methanol

To a mixture of 1-(tert-butyl) 3-methyl (E)-4-(fluoromethylene)-3-methylpiperidine-1,3-dicarboxylate (1.15 g, 4 mmol) in THF (50 mL) was added LiAlH₄ (304 mg, 8 mmol) at room temperature. The resulting mixture was stirred at 65° C. for 16 hrs. The reaction was then quenched with sodium sulfate decahydrate, filtered and concentrated to give the crude product (1.0 g), which was used directly in the next step without further purification. MS (ESI, m/e) [M+1]⁺ 174.1.

Steps 4-6: Examples 236 and 237 was prepared in similar fashion as Example 01 by replacing (2,2-difluorotetrahydro-1H-pyrrolizin-7a(5H)-yl)methanol with abovementioned (E)-(4-(fluoromethylene)-1,3-dimethylpiperidin-3-yl)methanol. The compound was further separated via chiral Prep-HPLC (Column: i-Cellulose-5, 21.2 mm×250 mm, 5 μm; Mobile Phase A: n-Hexane, Mobile Phase B: EtOH (with 0.2% 2M NH₃ in MeOH); Flow Rate: 18 mL/min; Room Temperature; Gradient: 40% B in 18 min) to give 2 isomers:

Example 236 (first eluting): ¹H NMR (500 MHz, CD₃OD) δ 6.75-6.58 (m, 2H), 6.47-6.41 (m, 1H), 4.60-4.10 (m, 7H), 3.71-3.57 (m, 2H), 2.88-2.67 (m, 3H), 2.42 (s, 3H), 2.27-2.23 (m, 4H), 2.10-1.75 (m, 8H), 1.21 (s, 3H). [M+H]⁺ 646.5.

Example 237 (second eluting): ¹H NMR (500 MHz, CD₃OD) δ 6.74-6.57 (m, 2H), 6.47-6.41 (m, 1H), 4.54-4.10 (m, 7H), 3.71-3.58 (m, 2H), 2.90-2.67 (m, 3H), 2.42 (s, 3H), 2.27-2.23 (m, 4H), 2.09-1.75 (m, 8H), 1.21 (s, 3H). [M+H]⁺ 646.5.

### Examples 238 and 239: 3-((6aR,7S,10R)-13-(((E)-1-(cyclopropylmethyl)-4-(fluoromethylene)-3-methylpiperidin-3-yl)methoxy)-1-fluoro-5,6,6a,7,8,9,10,11-octahydro-4-oxa-3,11a,12,14,15-pentaaza-7,10-methanocyclohepta[4,5]cycloocta[1,2,3-de]naphthalen-2-yl)-5-methyl-4-(trifluoromethyl)aniline

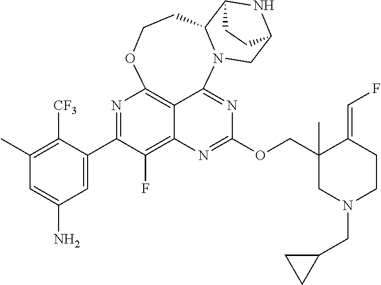 [SMILES: Cc1cc(N)cc(-c2nc3c4c(nc(OCC5(C)CN(CC6CC6)CC/C5=C\F)nc4c2F)N2C[C@H]4CC[C@H](N4)[C@H]2CCO3)c1C(F)(F)F]

### Synthetic Route

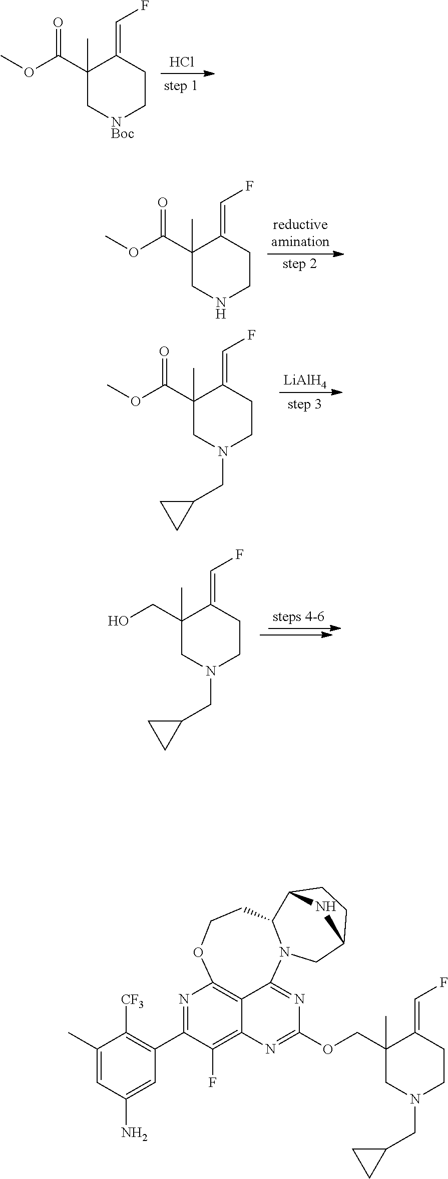

### Step 1: methyl (E)-4-(fluoromethylene)-3-methylpiperidine-3-carboxylate

To a solution of 1-(tert-butyl) 3-methyl (E)-4-(fluoromethylene)-3-methylpiperidine-1,3-dicarboxylate (287 mg, 1.0 mmol) in DCM (5 mL) was added HCl in dioxane (4 M, 2 ml) at room temperature. The resulting mixture was stirred at room temperature for 1 hour. The reaction mixture was then concentrated in vacuo to give the crude product (250 mg), which was used directly in the next step without further purification. MS (ESI, m/e) [M+1]⁺ 188.1.

### Step 2: methyl (E)-1-(cyclopropylmethyl)-4-(fluoromethylene)-3-methylpiperidine-3-carboxylate

To the mixture of methyl (E)-4-(fluoromethylene)-3-methylpiperidine-3-carboxylate (250 mg crude) in DCM (5 mL) were added cyclopropanecarbaldehyde (140 mg, 2 mmol) and sodium triacetoxyborohydride (423.9 mg, 2 mmol) at room temperature. The resulting mixture was stirred at room temperature for 2 hours. The reaction mixture was then quenched with saturated Na₂HCO₃ (20 mL), and extracted with DCM (15 mL×3). The organic layer was dried over anhydrous Na₂SO₄, filtered, and concentrated to give the crude residue. The crude was purified by silica gel column chromatography, eluted with 0-40% EA in PE to give the title product (200 mg, 0.83 mmol). MS (ESI, m/e) [M+1]⁺ 242.1.

### Step 3: (E)-(1-(cyclopropylmethyl)-4-(fluoromethylene)-3-methylpiperidin-3-yl)methanol

To a solution of methyl (E)-1-(cyclopropylmethyl)-4-(fluoromethylene)-3-methylpiperidine-3-carboxylate (200 mg, 0.83 mmol) in THF (5 mL) was added LiAlH₄ (38 mg, 1 mmol) at room temperature. The resulting mixture was stirred at room temperature for 1 hour. The reaction mixture was then quenched with sodium sulfate decahydrate, filtered, and concentrated to give the crude product (120 mg), which was used directly in the next step without further purification. MS (ESI, m/e) [M+1]⁺ 214.2.

Steps 4-6: Examples 238 and 239 was prepared in similar fashion as Example 01 by replacing (2,2-difluorotetrahydro-1H-pyrrolizin-7a(5H)-yl)methanol with abovementioned (E)-(1-(cyclopropylmethyl)-4-(fluoromethylene)-3-methylpiperidin-3-yl)methanol. The compound was further separated via chiral Prep-HPLC (Column: i-Cellulose-5, 21.2 mm×250 mm, 5 μm; Mobile Phase A: n-Hexane, Mobile Phase B: EtOH (with 0.2% 2M NH₃ in MeOH); Flow Rate: 18 mL/min; Room Temperature; Gradient: 40% B in 18 min) to give 2 isomers:

Example 238 (first eluting): ¹H NMR (500 MHz, CD₃OD) δ 6.74-6.57 (m, 2H), 6.42-6.41 (m, 1H), 4.56-4.08 (m, 7H), 3.73-3.59 (m, 2H), 3.11-2.68 (m, 2H), 2.42 (s, 3H), 2.27-1.81 (m, 12H), 1.22-1.15 (m, 4H), 0.90-0.47 (m, 4H). [M+H]⁺ 686.5.

Example 239 (second eluting): ¹H NMR (500 MHz, CD₃OD) δ 6.72-6.55 (m, 2H), 6.42-6.41 (m, 1H), 4.56-4.08 (m, 7H), 3.70-3.59 (m, 2H), 3.09-2.67 (m, 2H), 2.42 (s, 3H), 2.24-1.81 (m, 12H), 1.22-1.15 (m, 4H), 0.90-0.45 (m, 4H). [M+H]⁺ 686.5.

### Examples 240 and 241: 3-((6aR,7S,10R)-13-((4-(difluoromethyl)-3-methyl-1-(methyl-d3)piperidin-3-yl)methoxy-d2)-1-fluoro-5,6,6a,7,8,9,10,11-octahydro-4-oxa-3,11a,12,14,15-pentaaza-7,10-methanocyclohepta[4,5]cycloocta[1,2,3-de]naphthalen-2-yl)-5-methyl-4-(trifluoromethyl)aniline

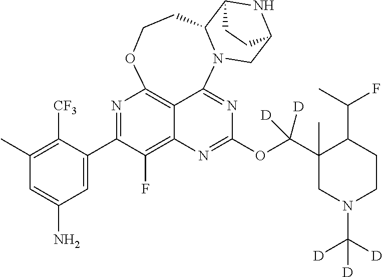 [SMILES: [2H]C([2H])([2H])N1CCC(C(C)F)C(C)(C([2H])([2H])Oc2nc3c4c(nc(-c5cc(N)cc(C)c5C(F)(F)F)c(F)c4n2)OCC[C@@H]2[C@@H]4CC[C@H](CN32)N4)C1]

### Synthetic Route

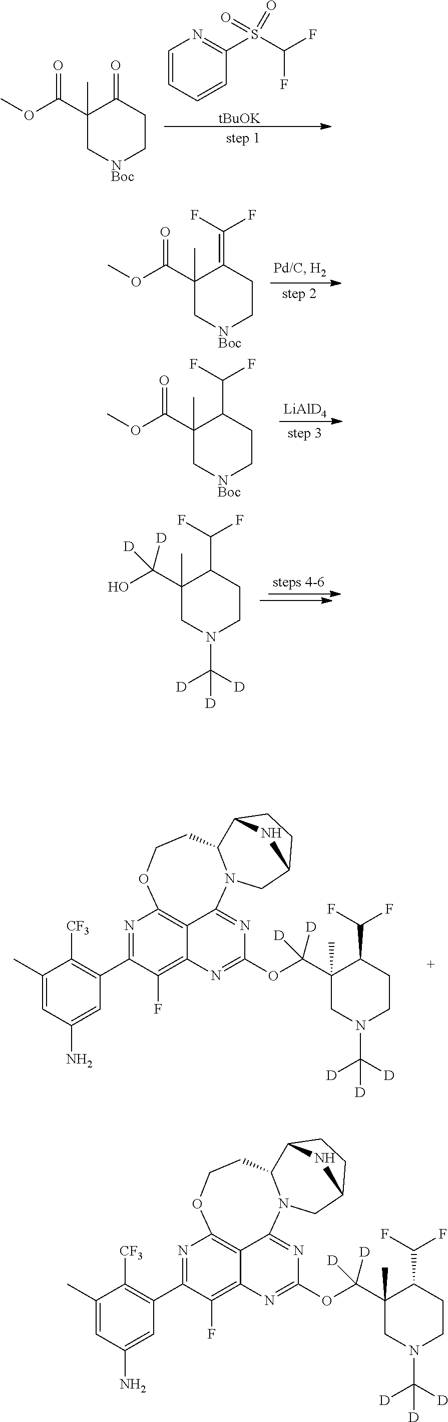

### Step 1: 1-(tert-butyl) 3-methyl 4-(difluoromethylene)-3-methylpiperidine-1,3-dicarboxylate

To the mixture of 2-((difluoromethyl)sulfonyl)pyridine (5.34 g, 27.65 mmol) and 1-(tert-butyl) 3-methyl 3-methyl-4-oxopiperidine-1,3-dicarboxylate (5.0 g, 18.43 mmol) in DMF (50 mL) was added t-BuOK (1 M, 33.17 mL) at −78° C. The resulting mixture was stirred at room temperature for 16 hours. The reaction mixture was quenched with saturated NH₄Cl (200 mL), and extracted with DCM (150 mL×3). The combined organic layer was dried over anhydrous Na₂SO₄, filtered and concentrated to give the crude residue. The crude was purified by silica gel column chromatography, eluted with 0-30% EA in PE to give the title product (1.2 g, 3.9 mmol). MS (ESI, m/e) [M+1]⁺ 306.1.

### Step 2: 1-(tert-butyl) 3-methyl 4-(difluoromethyl)-3-methylpiperidine-1,3-dicarboxylate

To the mixture of 1-(tert-butyl) 3-methyl 4-(difluoromethylene)-3-methylpiperidine-1,3-dicarboxylate (1.2 g, 3.9 mmol) in MeOH (5 mL) was added Pd/C (200 mg, 10% w/w). The resulting mixture was stirred at room temperature for 16 hours in a hydrogen atmosphere. The reaction mixture was then filtered and concentrated to give the crude residue. The residue was purified by silica gel column chromatography, eluted with 0-40% EA in PE to give the title product (1.0 g, 3.3 mmol). MS (ESI, m/e) [M+1]⁺ 308.2.

### Step 3: (4-(difluoromethyl)-3-methyl-1-(methyl-d3)piperidin-3-yl)methan-d2-ol

To a solution of 1-(tert-butyl) 3-methyl 4-(difluoromethyl)-3-methylpiperidine-1,3-dicarboxylate (300 mg, X mmol) in THF (15 mL) was added LiAlD₄ (76 mg, 2 mmol) at room temperature. The resulting mixture was stirred at 60° C. for 1 hour. The reaction mixture was cooled to room temperature and quenched with sodium sulfate decahydrate, filtered, and concentrated to give the crude product (200 mg), which was used directly in the next step without further purification. MS (ESI, m/e) [M+1]⁺ 199.2.

Steps 4-6: Examples 240 and 241 was prepared in similar fashion as Example 01 by replacing (2,2-difluorotetrahydro-1H-pyrrolizin-7a(5H)-yl)methanol with abovementioned (4-(difluoromethyl)-3-methyl-1-(methyl-d3)piperidin-3-yl)methan-d2-ol. The compound was further separated via chiral Prep-HPLC (Column: i-Cellulose-5, 21.2 mm×250 mm, 5 μm; Mobile Phase A: n-Hexane, Mobile Phase B: EtOH (with 0.2% 2M NH₃ in MeOH); Flow Rate: 18 mL/min; Room Temperature; Gradient: 40% B in 12 min) to give two 3,4-trans-isomers:

Example 240 (first eluting): ¹H NMR (500 MHz, CD₃OD) δ 6.69 (s, 1H), 6.43-6.41 (m, 1H), 6.33-6.11 (m, 1H), 4.27-4.10 (m, 5H), 3.77-3.63 (m, 2H), 3.16-3.05 (m, 2H), 2.42 (s, 3H), 2.20-1.81 (m, 11H), 1.23 (s, 3H). [M+H]⁺ 671.5.

Example 241 (second eluting): ¹H NMR (500 MHz, CD₃OD) δ 6.69 (s, 1H), 6.46-6.43 (m, 1H), 6.34-6.11 (m, 1H), 4.38-4.10 (m, 5H), 3.71-3.59 (m, 2H), 3.16-3.05 (m, 2H), 2.42 (s, 3H), 2.20-1.85 (m, 11H), 1.22 (s, 3H). [M+H]₊ 671.5.

Examples 242-247: The following examples were prepared in a manner similar to that in Examples 236-241.

| | | | 1H NMR | MS |
|---|---|---|---|---|
| Ex- | | | (500 MHz, | (ESI, m/e) |
| ample | Structure | Compound Name | CD₃OD) δ | [M + H]⁺ |
| 242 | 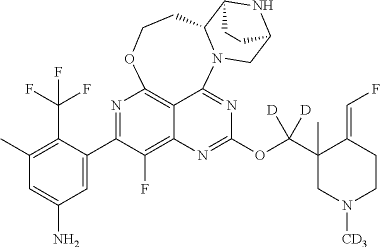 [SMILES: [2H]C([2H])([2H])N1CC/C(=C\F)C(C)(C([2H])([2H])Oc2nc3c4c(nc(-c5cc(N)cc(C)c5C(F)(F)F)c(F)c4n2)OCC[C@@H]2[C@@H]4CC[C@H](CN32)N4)C1] | 3-((6aR,7S,10R)-1-fluoro-13-(((E)- 4-(fluoromethylene)-3-methyl-1- (methyl-d3)piperidin-3-yl)methoxy)- d2)-5,6,6a,7,8,9,10,11-octahydro- 4-oxa-3,11a,12,14,15- pentaaza-7,10-methanocyclohepta [4,5]cycloocta[1,2,3-de]naphthalen- 2-yl)-5-methyl-4-(trifluoromethyl) aniline | 6.75-6.57 (m, 2H), 6.46-6.41 (m, 1H), 4.36-4.08 (m, 5H), 3.71-3.57 (m, 2H), 2.90-2.67 (m, 3H), 2.42 (s, 3H), 2.24- 2.23 (m, 1H), 2.03- 1.75 (m, 8H), 1.21 (s, 3H). | 651.5 |
| | (isomer 1) | | | |
| 243 | 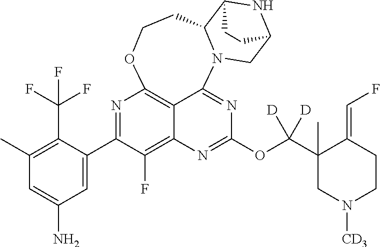 [SMILES: [2H]C([2H])([2H])N1CC/C(=C\F)C(C)(C([2H])([2H])Oc2nc3c4c(nc(-c5cc(N)cc(C)c5C(F)(F)F)c(F)c4n2)OCC[C@@H]2[C@@H]4CC[C@H](CN32)N4)C1] | 3-((6aR,7S,10R)-1-fluoro-13-(((E)- 4-(fluoromethylene)-3-methyl-1- (methyl-d3)piperidin-3-yl)methoxy- d2)-5,6,6a,7,8,9,10,11-octahydro- 4-oxa-3,11a,12,14,15- pentaaza-7,10-methanocyclohepta [4,5]cycloocta[1,2,3-de]naphthalen- 2-yl)-5-methyl-4-(trifluoromethyl) aniline | 6.73-6.55 (m, 2H), 6.46-6.41 (m, 1H), 4.37-4.08 (m, 5H), 3.71-3.55 (m, 2H), 2.88-2.65 (m, 3H), 2.42 (s, 3H), 2.31- 2.26 (m, 1H), 2.03- 1.75 (m, 8H), 1.20 (s, 3H). | 651.5 |
| | (isomer 2) | | | |
| 244 | 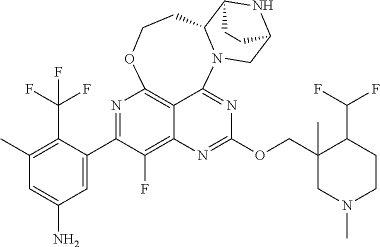 [SMILES: Cc1cc(N)cc(-c2nc3c4c(nc(OCC5(C)CN(C)CCC5C(F)F)nc4c2F)N2C[C@H]4CC[C@H](N4)[C@H]2CCO3)c1C(F)(F)F] | 3-((6aR,7S,10R)-13-((4- (difluoromethyl)-13- dimethylpiperidin-3-yl)methoxy)-1- fluoro-5,6,6a,7,8,9,10,11-octahydro- 4-oxa-3,11a,12,14,15- pentaaza-7,10-methanocyclohepta [4,5]cycloocta[1,2,3-de]naphthalen- 2-yl)-5-methyl-4-(trifluoromethyl) aniline | 6.69 (s, 1H), 6.46- 6.42 (m, 1H), 6.33- 6.11 (m, 1H), 4.53- 4.10 (m, 7H), 3.70- 3.63 (m, 2H), 3.11- 3.01 (m, 2H), 2.42 (s, 3H), 2.27 (s, 3H), 2.19-1.81 (m, 11H), 1.22 (s, 3H). | 666.5 |
| | (3,4-trans-piperidin-3-yl isomer 1) | | | |
| 245 | 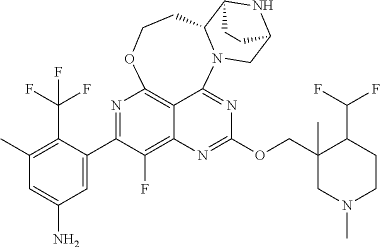 [SMILES: Cc1cc(N)cc(-c2nc3c4c(nc(OCC5(C)CN(C)CCC5C(F)F)nc4c2F)N2C[C@H]4CC[C@H](N4)[C@H]2CCO3)c1C(F)(F)F] | 3-((6aR,7S,10R)-13-((4- (difluoromethyl)-1,3- dimethylpiperidin-3-yl)methoxy)-1- fluoro-5,6,6a,7,8,9,10,11-octahydro- 4-oxa-3,11a,12,14,15- pentaaza-7,10-methanocyclohepta [4,5]cycloocta[1,2,3-de]naphthalen- 2-yl)-5-methyl-4-(trifluoromethyl) aniline | 6.69 (s, 1H), 6.43- 6.42 (m, 1H), 6.33- 6.11 (m, 1H), 4.51- 4.10 (m, 7H), 3.71- 3.63 (m, 2H), 3.11- 3.01 (m, 2H), 2.42 (s, 3H), 2.27 (s, 3H), 2.17-1.81 (m, 11H), 1.23 (s, 3H). | 666.5 |
| | (3,4-trans-piperidin-3-yl isomer 2) | | | |
| 246 | 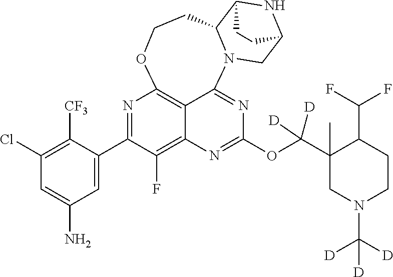 [SMILES: [2H]C([2H])([2H])N1CCC(C(F)F)C(C)(C([2H])([2H])Oc2nc3c4c(nc(-c5cc(N)cc(Cl)c5C(F)(F)F)c(F)c4n2)OCC[C@@H]2[C@@H]4CC[C@H](CN32)N4)C1] | 3-chloro-5-((6aR,7S,10R)-13-((4- (difluoromethyl)-3-methyl-1- (methyl-d3)piperidin-3-yl)methoxy- d2)-1-fluoro- 5,6,6a,7,8,9,10,11-octahydro- 4-oxa-3,11a,12,14,15- pentaaza-7,10-methanocyclohepta [4,5]cycloocta[1,2,3-de]naphthalen- 2-yl)-4-(trifluoromethyl) aniline | 6.89 (s, 1H), 6.55- 6.49 (m, 1H), 6.34- 6.11 (m, 1H), 4.28- 4.11 (m, 5H), 3.75- 3.63 (m, 2H), 3.16- 3.05 (m, 2H), 2.22- 1.81 (m, 11H), 1.22 (s, 3H). | 691.5 |
| | (3,4-trans-piperidin-3-yl isomer 1) | | | |
| 247 | 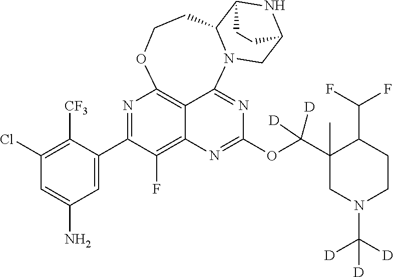 [SMILES: [2H]C([2H])([2H])N1CCC(C(F)F)C(C)(C([2H])([2H])Oc2nc3c4c(nc(-c5cc(N)cc(Cl)c5C(F)(F)F)c(F)c4n2)OCC[C@@H]2[C@@H]4CC[C@H](CN32)N4)C1] | 3-chloro-5-((6aR,7S,10R)-13-((4- (difluoromethyl)-d3-methyl-1- (methyl-d3)piperidin-3-yl)methoxy- d2)-1-fluoro- 5,6,6a,7,8,9,10,11-octahydro- 4-oxa-3,11a,12,14,15- pentaaza-7,10-methanocyclohepta [4,5]cycloocta[1,2,3-de]naphthalen- 2-yl)-4-(trifluoromethyl) aniline | 6.89 (s, 1H), 6.55- 6.49 (m, 1H), 6.33- 6.10 (m, 1H), 4.28- 4.11 (m, 5H), 3.75- 3.61 (m, 2H), 3.16- 3.05 (m, 2H), 2.22- 1.81 (m, 11H), 1.22 (s, 3H). | 691.5 |
| | (3,4-trans-piperidin-3-yl isomer 1) | | | |

### Example 248 and 249: 3-((6aR,7S,10R)-13-(((R)-1-((dimethylamino)methyl)-2,2-difluorocyclopropyl)methoxy)-1-fluoro-5-methyl-5,6,6a,7,8,9,10,11-octahydro-4-oxa-3,11a,12,14,15-pentaaza-7,10-methanocyclohepta[4,5]cycloocta[1,2,3-de]naphthalen-2-yl)-5-methyl-4-(trifluoromethyl)aniline

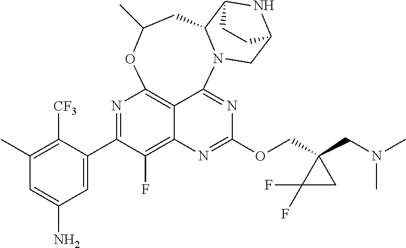

### Synthetic Route

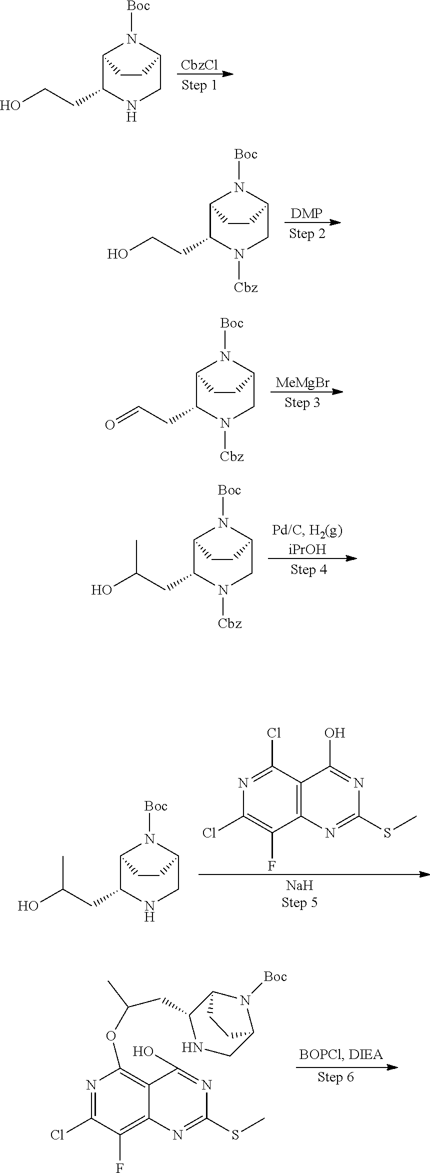

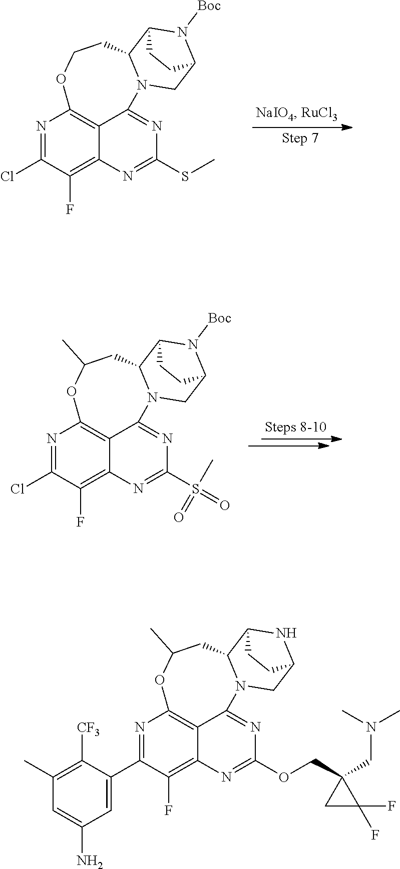

### Step 1: 3-benzyl 8-(tert-butyl) (1R,2R,5S)-2-(2-hydroxyethyl)-3,8-diazabicyclo[3.2.1]octane-3,8-dicarboxylate

To a mixture of tert-butyl (1R,2R,5S)-2-(2-hydroxyethyl)-3,8-diazabicyclo[3.2.1]octane-8-carboxylate (2.56 g, 10 mmol) and NaHCO₃ (2.52 g, 30 mmol) in a mixed solvent THF/H2O (8:2, 30 mL) was added benzyl carbonochloridate (1.87 g, 11 mmol) at room temperature and the mixture was stirred for 16 hours. The mixture was diluted with EA (100 mL), washed with brine (50 mL×2), dried over Na₂SO₄, filtered and concentrated. The resulted residue was purified by flash column chromatography to give the title product (3.59 g, 92%), which was used directly in the next step without further purification. MS (ESI, m/e) [M+23]⁺413.2.

### Step 2: 3-benzyl 8-(tert-butyl) (1R,2R,5S)-2-(2-oxoethyl)-3,8-diazabicyclo[3.2.1]octane-3,8-dicarboxylate

To a solution of 3-benzyl 8-(tert-butyl) (1R,2R,5S)-2-(2-hydroxyethyl)-3,8-diazabicyclo[3.2.1]octane-3,8-dicarboxylate (3.59 g, 9.2 mmol) in CH₂Cl₂ (40 mL) was added DMP (5.10 g, 12.0 mmol) at room temperature. The resulting mixture was stirred for 16 hours. The mixture was diluted with CH₂Cl₂ (100 mL), washed with NaHCO₃ (50 mL), dried over Na₂SO₄, filtered and concentrated. The crude was purified by flash column chromatography to give the title product (2.64 g, 74%). MS (ESI, m/e) [M+1]⁺ 389.3.

### Step 3: 3-benzyl 8-(tert-butyl) (1R,2R,5S)-2-(2-hydroxypropyl)-3,8-diazabicyclo[3.2.1]octane-3,8-dicarboxylate

To a solution of 3-benzyl 8-(tert-butyl) (1R,2R,5S)-2-(2-oxoethyl)-3,8-diazabicyclo[3.2.1]octane-3,8-dicarboxylate (2.5 g, 6.44 mmol) in THF (50 mL) was added MeMgBr (3.0 M, 4.2 mL, 12.6 mmol) at 0° C. dropwise under a nitrogen atmosphere. The resulting mixture was stirred at room temperature for 2 hours. The reaction was quenched with sat. NH₄Cl (50 mL), extracted with EtOAc (50 mL×3). The combined extracts were washed with brine (50 mL×2), dried over Na₂SO₄, filtered and concentrated. The residue was purified by flash column chromatography to give the title product (720 mg, 28%). MS (ESI, m/e) [M+23]⁺ 427.3.

### Step 4: tert-butyl (1R,2R,5S)-2-(2-hydroxypropyl)-3,8-diazabicyclo[3.2.1]octane-8-carboxylate

A mixture of 3-benzyl 8-(tert-butyl) (1R,2R,5S)-2-(2-hydroxypropyl)-3,8-diazabicyclo[3.2.1]octane-3,8-dicarboxylate (700 mg, 1.73 mmol) and Pd/C (200 mg, 5% wet) in isopropanol (20 mL) was stirred under a nitrogen atmosphere at room temperature for 16 hours. The mixture was then filtered and concentrated in vacuo to give the title product (480 mg, 100%), which was used directly in the next step without further purification. MS (ESI, m/e) [M+1]⁺ 271.2.

### Step 5: tert-butyl (1S,2R,5R)-2-(2-((7-chloro-8-fluoro-4-hydroxy-2-(methylthio)pyrido[4,3-d]pyrimidin-5-yl)oxy)propyl)-3,8-diazabicyclo[3.2.1]octane-8-carboxylate

To a mixture of tert-butyl (1R,2R,5S)-2-(2-hydroxypropyl)-3,8-diazabicyclo[3.2.1]octane-8-carboxylate (450 mg, 1.67 mmol) and 5,7-dichloro-8-fluoro-2-(methylthio)pyrido[4,3-d]pyrimidin-4-ol (470 mg, 1.67 mmol) in THF (20 mL) was added NaH (333 mg, 8.33 mmol). The mixture was stirred at room temperature for 16 hours. The reaction was then quenched by addition of saturated ammonium chloride aqueous solution (20 mL). The resulting mixture was extracted by EtOAc (20 mL×3). The combined organic phase was dried over Na₂SO₄, filtered and concentrated in vacuo to give the crude title product (851 g), which was used directly without further purification. MS (ESI, m/e) [M+1]⁺ 514.2.

### Step 6: tert-butyl (6aR,7S,10R)-2-chloro-1-fluoro-5-methyl-13-(methylthio)-5,6,6a,7,8,9,10,11-octahydro-4-oxa-3,11a,12,14,15-pentaaza-7,10-methanocyclohepta[4,5]cycloocta[1,2,3-de]naphthalene-15-carboxylate

To a solution of tert-butyl (1S,2R,5R)-2-(2-((7-chloro-8-fluoro-4-hydroxy-2-(methylthio)pyrido[4,3-d]pyrimidin-5-yl)oxy)propyl)-3,8-diazabicyclo[3.2.1]octane-8-carboxylate (800 mg, 1.56 mmol) in MeCN (220 mL) was added DIPEA (402 mg, 3.12 mmol) and BOP—Cl (515 mg, 2.03 mmol). The resulting mixture was stirred at 70° C. for 1 hour. The mixture was diluted with EA (30 mL), washed with brine (15 mL×2), dried over Na₂SO₄, filtered and concentrated. The crude was purified by column chromatography to give the title product (720 mg, 93%). MS (ESI, m/e) [M+1]⁺ 496.3.

### Step 7: tert-butyl (6aR,7S,10R)-2-chloro-1-fluoro-5-methyl-13-(methylsulfonyl)-5,6,6a,7,8,9,10,11-octahydro-4-oxa-3,11a,12,14,15-pentaaza-7,10-methanocyclohepta[4,5]cycloocta[1,2,3-de]naphthalene-15-carboxylate

To a solution of tert-butyl (6aR,7S,10R)-2-chloro-1-fluoro-5-methyl-13-(methylthio)-5,6,6a,7,8,9,10,11-octahydro-4-oxa-3,11a,12,14,15-pentaaza-7,10-methanocyclohepta[4,5]cycloocta[1,2,3-de]naphthalene-15-carboxylate (720 mg, 1.45 mmol) in a mixed solvent of THF/H₂O (4:1, 40 mL) was added NaIO₄ (915 mg, 4.35 mmol) and RuCl₃ (28 mg, 0.14 mmol) at 0° C. The resulting mixture was stirred at 0° C. for 1 hour. The mixture was then diluted with water (20 mL), extracted by EA (20 mL×2). The combined organic phase was dried over Na₂SO₄, and filtered and concentrated in vacuo. The crude was purified by column chromatography to give the title product (620 mg, 81%). MS (ESI, m/e) [M+1]⁺ 528.2.

Steps 8-10: Examples 248 and 249 was prepared in similar fashion as Example 45 by replacing the corresponding coupling partners. The mixture of methyl isomers was further separated via prep-TLC (CH₂Cl₂/MeOH (w/NH₃)=8:1) to give first eluting isomer (8 mg, 28%) as Example 248 and second eluting isomer (3 mg, 10%) as Example 249:

Example 248: ¹H NMR (400 MHz, CD₃OD) δ 6.68 (s, 1H), 6.42 (d, J=59.6 Hz, 1H), 4.67-4.51 (m, 2H), 4.50-4.37 (m, 1H), 4.35-4.16 (m, 1H), 3.99-3.39 (m, 3H), 3.29-3.16 (m, 1H), 2.96-2.79 (m, 1H), 2.54-2.45 (m, 1H), 2.44-2.16 (m, 9H), 2.11-1.57 (m, 6H), 1.54-1.24 (m, 5H). MS (ESI, m/e) [M+1]⁺ 652.5.

Example 249: ¹H NMR (400 MHz, CD₃OD) δ 6.69 (s, 1H), 6.42 (d, J=51.6 Hz, 1H), 4.84-4.49 (m, 3H), 4.28-3.76 (m, 2H), 3.74-3.61 (m, 1H), 3.29-3.19 (m, 2H), 3.07-2.88 (m, 1H), 2.71-2.50 (m, 1H), 2.52-2.26 (m, 6H), 2.25-1.96 (m, 2H), 1.94-1.64 (m, 3H), 1.64-1.20 (m, 8H), 0.96-0.81 (m, 1H). MS (ESI, m/e) [M+1]⁺ 652.5.

### Example 250: 3-((6aR,7S,10R)-1-fluoro-13-((S)-1-((S)-1-(2-fluoroethyl)pyrrolidin-2-yl)ethoxy)-5,6,6a,7,8,9,10,11-octahydro-4-oxa-3,11a,12,14,15-pentaaza-7,10-methanocyclohepta[4,5]cycloocta[1,2,3-de]naphthalen-2-yl)-5-methyl-4-(trifluoromethyl)aniline

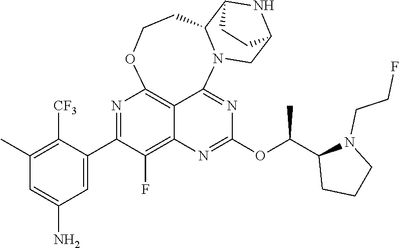 [SMILES: Cc1cc(N)cc(-c2nc3c4c(nc(O[C@@H](C)[C@@H]5CCCN5CCF)nc4c2F)N2C[C@H]4CC[C@H](N4)[C@H]2CCO3)c1C(F)(F)F]

### Synthetic Route

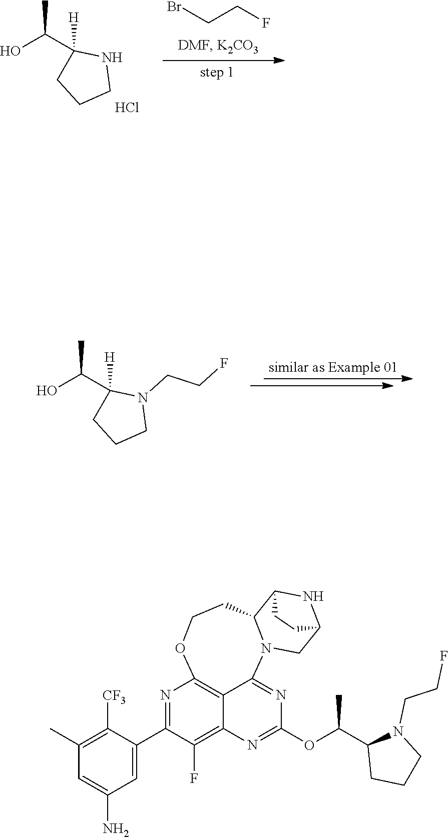

### Step 1: (S)-1-((S)-1-(2-fluoroethyl)pyrrolidin-2-yl)ethan-1-ol

To a solution of (S)-1-((S)-pyrrolidin-2-yl)ethan-1-ol hydrochloride (70 mg, 0.46 mmol) in DMF (2 mL) was added 1-bromo-2-fluoroethane (70 mg, 0.56 mmol) and K₂CO₃ (190 mg, 1.38 mmol) at room temperature. The resulting mixture was stirred at room temperature for 16 hours. The mixture was diluted with EtOAc (20 mL), and washed with H₂O (20 mL×3) and brine (20 mL). The organic phase was dried over Na₂SO₄ and concentrated in vacuo. The resulting residue was purified by silica gel column chromatography (DCM/MeOH=10:1) to give the title product (20 mg). MS (ESI, m/e) [M+1]⁺ 162.3.

Steps 2-4: Example 250 was prepared in similar fashion as Example 01 by replacing (2,2-difluorotetrahydro-1H-pyrrolizin-7a(5H)-yl)methanol with abovementioned (S)-1-((S)-1-(2-fluoroethyl)pyrrolidin-2-yl)ethan-1-ol. ¹H NMR (500 MHz, CD₃OD) δ 6.69 (s, 1H), 6.46-6.41 (m, 1H), 5.31-5.27 (m, 1H), 4.58-4.50 (m, 6H), 4.50-4.41 (m, 1H), 4.38-4.21 (m, 2H), 4.19-3.98 (m, 2H), 3.92-3.66 (m, 3H), 3.10-2.75 (m, 1H), 2.42-2.38 (m, 3H), 2.30-2.06 (m, 2H), 2.04-1.73 (m, 7H), 1.39 (d, J=6.3 Hz, 3H). MS (ESI, m/e) [M+1]⁺ 634.5.

### Assays

### KRAS WT and KRAS G12D Probe Displacement Assay

This assay was used to identify compounds which bind to GDP-loaded KRAS protein and are able to displace a biotinylated probe occupying the KRAS binding site. GST-tagged GDP-loaded WT KRAS (amino acids 1-188) and GST-tagged GDP-loaded KRAS G12D (amin acids 1-188) were expressed in E. coli and purified in house. All protein and reaction solutions were prepared in assay buffer containing 50 mM HEPES pH7.5, 50 mM NaCl, 1 mM MgCl₂, 1 mM TCEP, 0.01% BSA, and 0.008% Brij-35. Purified WT KRAS (3 nM final concentration) or KRAS G12D protein (0.5 nM final concentration) was incubated with a 3-fold serially diluted compound in the assay plate (384 well microplate, black, Corning). Plates are incubated at 24° C. for 1 hr. Following the incubation, biotinylated probe 1 (60 nM final assay concentration) for WT KRAS and biotinylated probe 2 (4 nM final assay concentration) for KRAS G12D was added to the assay plate, respectively. After 1 hr incubation at 24° C., Mab Anti-GST-Tb cryptate (Cisbio) and Streptavidin-XL665 (Cisbio) were added and further incubated at 24° C. for another 1 hr. The TR-FRET signals (ex337 nm, em665 nm/620 nm) were read on BMG PHERAstar FSX instrument. The inhibition percentage of KRAS protein binding with biotinylated probe in presence of increasing concentrations of compounds was calculated based on the ratio of fluorescence at 665 nm to that at 620 nm. The IC₅₀ value of each compound was calculated from fitting the data to the four-parameter logistic model by Dotmatics.

The synthetic routes of probe 1 and 2, which have been disclosed in PCT/CN2022/070676, are hereby incorporated herein.

### Proble 1: N-(2-(2-(3-(3-((S)-2-(((7-(8-chloronaphthalen-1-yl)-4-((S)-3-(cyanomethyl)-4-(methylsulfonyl)piperazin-1-yl)-5,6,7,8-tetrahydropyrido[3,4-d]pyrimidin-2-yl)oxy)methyl)pyrrolidin-1-yl)propoxy)propanamido)ethoxy)ethyl)-5-((3aS,4S,6aR)-2-oxohexahydro-1H-thieno[3,4-d]imidazol-4-yl)pentanamide

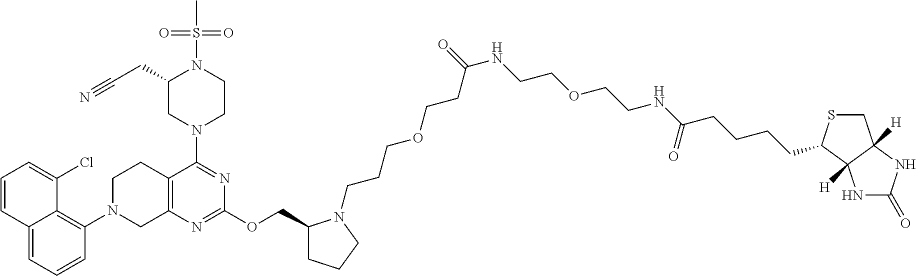 [SMILES: CS(=O)(=O)N1CCN(c2nc(OC[C@@H]3CCCN3CCCOCCC(=O)NCCOCCNC(=O)CCCC[C@@H]3SC[C@@H]4NC(=O)N[C@@H]43)nc3c2CCN(c2cccc4cccc(Cl)c24)C3)C[C@@H]1CC#N]

### Synthetic Route

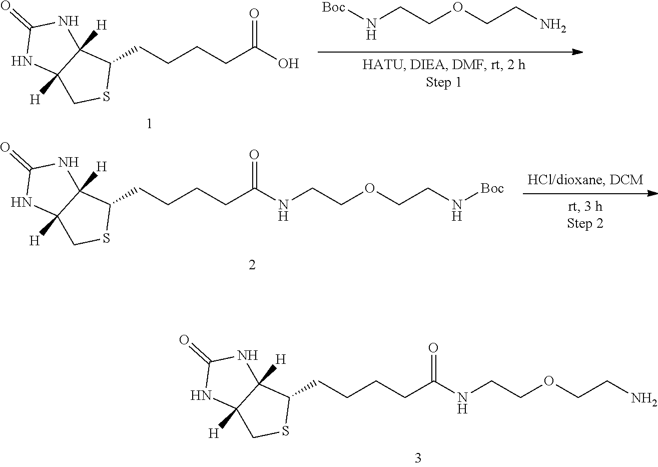 [SMILES: O=C(O)CCCC[C@@H]1SC[C@@H]2NC(=O)N[C@@H]21 ; CC(C)(C)OC(=O)NCCOCCN ; CC(C)(C)OC(=O)NCCOCCNC(=O)CCCC[C@@H]1SC[C@@H]2NC(=O)N[C@@H]21 ; NCCOCCNC(=O)CCCC[C@@H]1SC[C@@H]2NC(=O)N[C@@H]21]

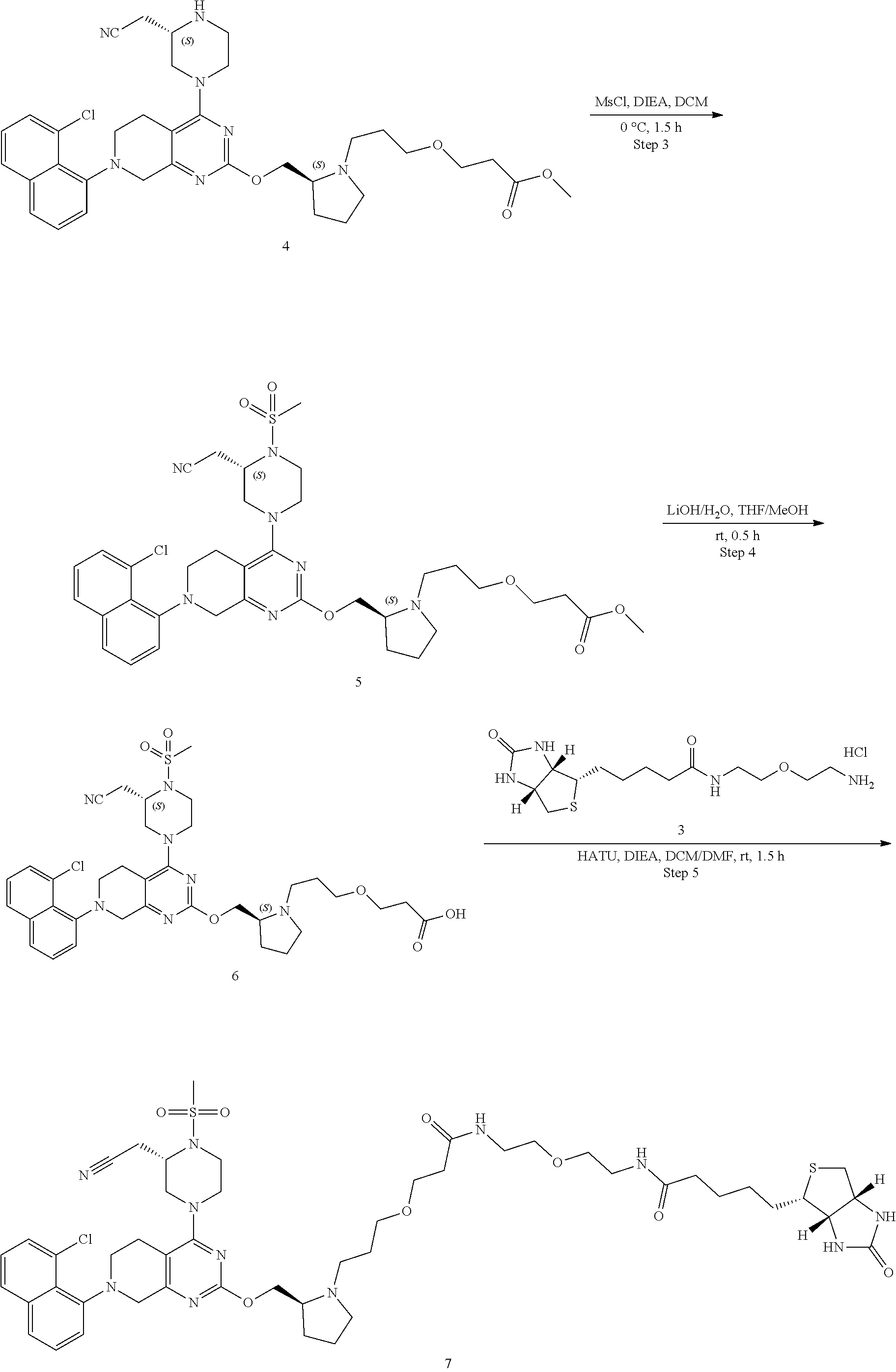

### Step 1: tert-butyl (2-(2-(5-((3aS,4S,6aR)-2-oxohexahydro-1H-thieno[3,4-d]imidazol-4-yl)pentanamido)ethoxy)ethyl)carbamate

To a solution of 5-((3aS,4S,6aR)-2-oxohexahydro-1H-thieno[3,4-d]imidazol-4-yl)pentanoic acid (244 mg, 1 mmol) in DMF (20 mL) was added HATU (380 mg, 1.2 mmol), DIPEA (380 mg, 3 mmol) and tert-butyl (2-(2-aminoethoxy)ethyl)carbamate (204 mg, 1 mmol), stirred at room temperature for 2 hours. The resulting solution was concentrated and purified by combi-flash (DCM/MeOH/NH₃H₂O=10/1/0.1) to give the title product (430 mg, 100%). MS (ESI, m/e) [M+H]⁺ 431.3.

### Step 2: N-(2-(2-aminoethoxy)ethyl)-5-((3aS,4S,6aR)-2-oxohexahydro-1H-thieno[3,4-d]imidazol-4-yl)pentanamide

To a solution of tert-butyl (2-(2-(5-((3aS,4S,6aR)-2-oxohexahydro-1H-thieno[3,4-d]imidazol-4-yl)pentanamido)ethoxy)ethyl)carbamate (100 mg, 0.23 mmol) in DCM (5 mL) was added 4M HCl/dioxane (2.5 mL) and stirred at room temperature for 1 hour. The resulting solution was concentrated to give the title product (100 mg, 30%, HCl salt) as white solid. MS (ESI, m/e) [M+H]⁺ 331.2.

### Step 3: methyl 3-(3-((S)-2-(((7-(8-chloronaphthalen-1-yl)-4-((S)-3-(cyanomethyl)-4-(methylsulfonyl)piperazin-1-yl)-5,6,7,8-tetrahydropyrido[3,4-d]pyrimidin-2-yl)oxy)methyl)pyrrolidin-1-yl)propoxy)propanoate

To a solution of methyl 3-(3-((S)-2-(((7-(8-chloronaphthalen-1-yl)-4-((S)-3-(cyanomethyl)piperazin-1-yl)-5,6,7,8-tetrahydropyrido[3,4-d]pyrimidin-2-yl)oxy)methyl)pyrrolidin-1-yl)propoxy)propanoate (290 mg, 0.44 mmol) in DCM was added DIPEA (375 mg, 2.91 mmol) and MsCl (200 mg, 1.75 mmol), stirred at 0° C. for 1.5 hours. The resulting solution was washed with brine (30 mL*2) and dried over Na₂SO₄, the solution was concentrated and purified by Pre-TLC (DCM/MeOH/NH₃H₂O=10/1/0.1) to give the title product (130 mg, 40%). MS (ESI, m/e) [M/2+H]+ 740.4.

### Step 4: 3-(3-((S)-2-(((7-(8-chloronaphthalen-1-yl)-4-((S)-3-(cyanomethyl)-4-(methylsulfonyl)piperazin-1-yl)-5,6,7,8-tetrahydropyrido[3,4-d]pyrimidin-2-yl)oxy)methyl)pyrrolidin-1-yl)propoxy)propanoic acid

To a solution of methyl 3-(3-((S)-2-(((7-(8-chloronaphthalen-1-yl)-4-((S)-3-(cyanomethyl)-4-(methylsulfonyl)piperazin-1-yl)-5,6,7,8-tetrahydropyrido[3,4-d]pyrimidin-2-yl)oxy)methyl)pyrrolidin-1-yl)propoxy)propanoate (130 mg, 0.18 mmol) in methanol (5 mL) was added THF (5 mL) and LiOH/H₂O (1M, 2.5 mL), stirred at room temperature for 0.5 hour. Then it was neutralized by HCl/H₂O (1M) to pH=5-6, and the solution was evaporated, dissolved in DCM (10 mL) and filtered the solid, the filtrate was concentrated and dried to give the title product (127 mg, 99%). MS (ESI, m/e) [M+H]⁺ 726.4.

### Step 5: N-(2-(2-(3-(3-((S)-2-(((7-(8-chloronaphthalen-1-yl)-4-((S)-3-(cyanomethyl)-4-(methylsulfonyl)piperazin-1-yl)-5,6,7,8-tetrahydropyrido[3,4-d]pyrimidin-2-yl)oxy)methyl)pyrrolidin-1-yl)propoxy)propanamido)ethoxy)ethyl)-5-((3aS,4S,6aR)-2-oxohexahydro-1H-thieno[3,4-d]imidazol-4-yl)pentanamide

To a mixture of 3-(3-((S)-2-(((7-(8-chloronaphthalen-1-yl)-4-((S)-3-(cyanomethyl)-4-(methylsulfonyl)piperazin-1-yl)-5,6,7,8-tetrahydropyrido[3,4-d]pyrimidin-2-yl)oxy)methyl)pyrrolidin-1-yl)propoxy)propanoic acid (100 mg, 0.14 mmol), N-(2-(2-aminoethoxy)ethyl)-5-((3aS,4S,6aR)-2-oxohexahydro-1H-thieno[3,4-d]imidazol-4-yl)pentanamide hydrochloride (46 mg, 0.14 mmol) and HATU (79 mg, 0.21 mmol) was added DCM (10 mL) and DMF (5 mL), then DIPEA (225 mg, 1.74 mmol) was added and stirred at room temperature for 1.5 hours. The resulting solution was washed with water (20 mL each, 3 times) and dried over Na₂SO₄, the solution was concentrated and purified by Pre-HPLC to give the title product (5.92 mg, 4%) as a FA salt. ¹H NMR (500 MHz, CD₃OD) δ 8.35 (bs, 2H), 7.84-7.83 (m, 1H), 7.70-7.68 m, 1H), 7.554-7.48 (m, 2H), 7.40-7.31 (m, 2H), 4.74-4.70 (m, 2H), 4.50-4.39 (m, 2H), 4.37-4.31 (m, 1H), 4.28-4.16 (m, 2H), 4.06-4.04 (m, 1H), 3.96-3.90 (m, 1H), 3.82-3.56 (m, 10H), 3.52-3.42 (m, 5H), 3.38-23.33 (m, 2H), 3.28-3.11 (m, 7H), 3.10-3.04 (m, 4H), 2.96-2.95 (m, 1H), 2.91-2.88 (m, 1H), 2.76-2.65 (m, 2H), 2.49-2.29 (m, 3H), 2.25-2.09 (m, 4H), 2.09-1.92 (m, 4H), 1.77-1.49 (m, 4H), 1.44-1.38 (m, 2H). MS (ESI, m/e) [M/2+H]⁺ 520.0.

### Proble 2: N-(2-(2-(3-(3-(4-(3-((7-(2-amino-7-fluorobenzo[d]thiazol-4-yl)-6-chloro-8-fluoro-4-(piperazin-1-yl)quinazolin-2-yl)oxy)propyl)piperazin-1-yl)propoxy)propanamido)ethoxy)ethyl)-5-((3aS,4S,6aR)-2-oxohexahydro-1H-thieno[3,4-d]imidazol-4-yl)pentanamide

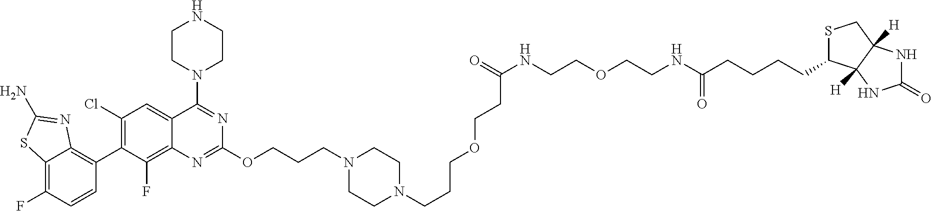 [SMILES: Nc1nc2c(-c3c(Cl)cc4c(N5CCNCC5)nc(OCCCN5CCN(CCCOCCC(=O)NCCOCCNC(=O)CCCC[C@@H]6SC[C@@H]7NC(=O)N[C@@H]76)CC5)nc4c3F)ccc(F)c2s1]

### Synthetic Route

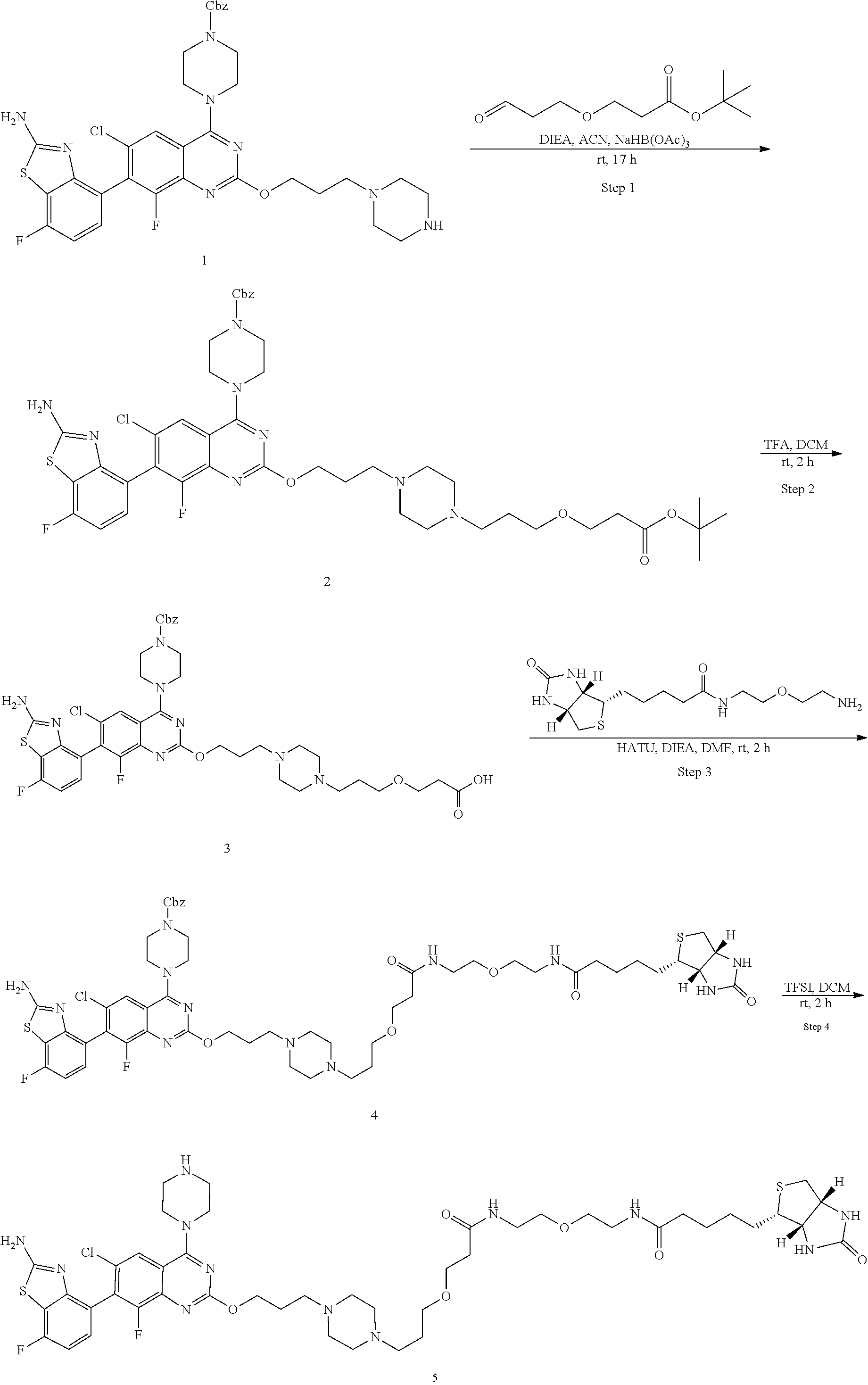

### Step 1: benzyl 4-(7-(2-amino-7-fluorobenzo[d]thiazol-4-yl)-2-(3-(4-(3-(3-(tert-butoxy)-3-oxopropoxy)propyl)piperazin-1-yl)propoxy)-6-chloro-8-fluoroquinazolin-4-yl)piperazine-1-carboxylate

To a mixture of benzyl 4-(7-(2-amino-7-fluorobenzo[d]thiazol-4-yl)-6-chloro-8-fluoro-2-(3-(piperazin-1-yl)propoxy)quinazolin-4-yl)piperazine-1-carboxylate (1 g, 1.4 mmol) in CH₃CN (100 mL) was added DIPEA (300 mg, 2.3 mmol), and NaHB(AcO)₃ (600 mg, 2.8 mmol), stirred at room temperature for 17 hours. The resulting mixture was filtered with celite and the filtrate was concentrated and purified by combi-flash (DCM/MeOH/NH₃H₂O=10/1/0.1) to give the title product (800 mg, 64%). MS (ESI, m/e) [M+H]⁺ 895.7

### Step 2: 3-(3-(4-(3-((7-(2-amino-7-fluorobenzo[d]thiazol-4-yl)-4-(4-((benzyloxy)carbonyl)piperazin-1-yl)-6-chloro-8-fluoroquinazolin-2-yl)oxy)propyl)piperazin-1-yl)propoxy)propanoic acid

To a solution of benzyl 4-(7-(2-amino-7-fluorobenzo[d]thiazol-4-yl)-2-(3-(4-(3-(3-(tert-butoxy)-3-oxopropoxy)propyl)piperazin-1-yl)propoxy)-6-chloro-8-fluoroquinazolin-4-yl)piperazine-1-carboxylate (800 mg, 0.9 mmol) in DCM (50 mL) was added TFA (10 mL) and stirred at room temperature for 2 hours. The resulting solution was concentrated to give the title product (1 g, crude) as thick brown oil used directly in next step. MS (ESI, m/e) [M+H]⁺ 839.6

### Step 3: benzyl 4-(7-(2-amino-7-fluorobenzo[d]thiazol-4-yl)-6-chloro-2-(3-(4-(7,15-dioxo-19-((3aS,4S,6aR)-2-oxohexahydro-1H-thieno[3,4-d]imidazol-4-yl)-4,11-dioxa-8,14-diazanonadecyl)piperazin-1-yl)propoxy)-8-fluoroquinazolin-4-yl)piperazine-1-carboxylate

To a solution of 3-(3-(4-(3-((7-(2-amino-7-fluorobenzo[d]thiazol-4-yl)-4-(4-((benzyloxy)carbonyl)piperazin-1-yl)-6-chloro-8-fluoroquinazolin-2-yl)oxy)propyl)piperazin-1-yl)propoxy)propanoic acid (200 mg, 0.24 mmol), N-(2-(2-aminoethoxy)ethyl)-5-((3aS,4S,6aR)-2-oxohexahydro-1H-thieno[3,4-d]imidazol-4-yl)pentanamide (80 mg, 0.24 mmol) and HATU (136 mg, 0.36 mmol) in DMF (15 mL) was added DIPEA (92 mg, 0.72 mmol) and stirred at room temperature for 2 hours. The resulting solution was concentrated and purified by Pre-TLC (DCM/MeOH/NH₃H₂O=10/1/0.1) to give the title product (100 mg, 36%) as brown solid. MS (ESI, m/e) [M/2+H]⁺ 576.5

### Step 4: N-(2-(2-(3-(3-(4-(3-((7-(2-amino-7-fluorobenzo[d]thiazol-4-yl)-6-chloro-8-fluoro-4-(piperazin-1-yl)quinazolin-2-yl)oxy)propyl)piperazin-1-yl)propoxy)propanamido)ethoxy)ethyl)-5-((3aS,4S,6aR)-2-oxohexahydro-1H-thieno[3,4-d]imidazol-4-yl)pentanamide

A solution of benzyl 4-(7-(2-amino-7-fluorobenzo[d]thiazol-4-yl)-6-chloro-2-(3-(4-(7,15-dioxo-19-((3aS,4S,6aR)-2-oxohexahydro-1H-thieno[3,4-d]imidazol-4-yl)-4,11-dioxa-8,14-diazanonadecyl)piperazin-1-yl)propoxy)-8-fluoroquinazolin-4-yl)piperazine-1-carboxylate (90 mg, 0.08 mmol) in DCM (20 mL) was added TMSI (200 mg, 1 mmol), stirred at 25° C. for 2 hours. The resulting solution was concentrated and purified by Prep-HPLC to give the title product (44 mg, 54%, FA salt) as white solid. ¹H NMR (500 MHz, CD₃OD) δ 8.36 (s, 1H), 7.93 (s, 1H), 7.24-7.21 (m, 1H), 7.02-6.99 (m, 1H), 4.59-4.51 (m, 2H), 4.49-4.46 (m, 1H), 4.30-4.27 (m, 1H), 4.07-4.05 (m, 4H), 3.69-3.67 (m, 2H), 3.59-3.56 (m, 2H), 3.53-3.50 (m, 4H), 3.47-3.45 (m, 4H), 3.38-3.33 (m, 4H), 3.20-3.06 (m, 7H), 2.93-2.78 (m, 7H), 2.70-2.66 (m, 1H), 2.48-2.45 (m, 2H), 2.22-2.19 (m, 2H), 2.12-2.07 (m, 2H), 1.96-1.91 (m, 2H), 1.75-1.53 (m, 4H), 1.45-1.39 (m, 2H). MS (ESI, m/e) [M+H]⁺ 1017.7.

### Metabolic Stability in Different Species of Liver Microsome

Liver microsomes were first mixed with NADPH to obtain final concentrations of microsomes and NADPH of 0.5 mg/mL and 1 mM, respectively. Test compounds was added to incubation system at final concentration of 1 μM and incubated at 37° C. The incubation was initiated by the addition of NADPH into the system. Aliquots of 20 μL were taken from the incubation system at 0, 15, 30, 45 and 60 min after the initiation of incubation. The reaction solutions were stopped by the addition of cold acetonitrile with analytical IS. Samples were centrifuged at 4000 rpm for 5 minutes and were then analyzed on LC-MS/MS.

Peak areas of samples from various timepoints were determined from extracted ion chromatograms; and were then plotted to calculate metabolic stability. The slope value, k, was determined by linear regression of the natural logarithm of the remaining percentage of the parent drug vs. incubation time curve. The in vitro half-life (in vitro t_{1/2}) was determined from the slope value:

in vitro t_{1/2}=−(0.693%)

Conversion of the in vitro t_{1/2} (in min) into the in vitro intrinsic clearance (in vitro CLᵢₙₜ, in μL/min/mg proteins) was done using the following equation (mean of duplicate determinations):

$$in vitro {CL}_{i n t} = \frac{0.693}{{t}_{1 / 3}} \times \frac{volume of incubation ( μL )}{amount of proteins ( mg )}$$

The control compound (verapamil) was included in the assay to ensure the data consistency. The negative control (identical experimental set-up but no NADPH in the incubation system) was used to exclude the misleading factor that resulted from instability of chemical itself.

### CYP (Cytochrome P450) Enzymes Inhibition Assay in Human Liver Microsome

The incubation was carried out in 96-well plates. 1 μL of test compound working solution or vehicle was added into 179 μL of human liver microsomes fortified with substrates of CYP1A2 (40 μM phenacetin), 2C9 (6 μM diclofenac), 2C19 (50 μM (S)-mephenytoin), 2D6 (10 μM dextromethorphan) and 3A4 (1 M midazolam or 50 μM testosterone). The incubation plate was pre-warmed at 37° C. for 5 min in water bath before the reactions are started by the addition of 20 μL of 10 mM NADPH solution. The reaction was carried out in the 37° C.-water bath.

At the predetermined time points, the reaction was stopped by adding 300 μL of quenching solution (acetonitrile with internal standards) to each well. The sample plate was vortexed for 1 min and centrifuged at 3000 g for 10 min. 100 μL of the supernatant was transferred to a new 96-well plate then mixed with 100 μL water for analysis by LC-MS/MS followed by data processing (i.e., percent inhibition at 10 uM or IC50 determination).

### Time Dependent Cytochrome P450 (CYP) Enzymes Inhibition Assay (TDI) in Human Liver Microsome

The TDI assay involves pre-incubation (“inactivation incubation”) of 0.1 mg·mL-1 human liver microsome with 10 uM test compounds and Positive Control in the presence or absence of 1 mM NADPH at 37° C. for 30 min. Following the pre-incubation period, remaining CYP activity was determined by subsequently adding substrates (1A2, 40 μM phenacetin; CYP2B6, 50 μM bupropion; CYP2C8, 5 μM paclitaxel; CYP2C9, 6 μM diclofenac; CYP2C19, 50 μM (S)-mephenytoin; CYP2D6, 10 μM dextromethorphan, CYP3A, 1 μM Midazolam or 50 μM Testosterone) and NADPH to the pre-incubation mixtures and an “activity incubation” was done for another 20 min for CYP1A2, 2B6, 2C19, 2D6, 10 min for CYP2C8, CYP3A (testosterone), 6 min for CYP2C9 and 5 min for 3A (midazolam). All reactions are terminated by the addition of ice-cold acetonitrile with internal standard and then centrifuge for LC-MS/MS analysis.

### Bidirectional Permeability Assay in MDCKII-MDR1 Cell Monolayer

MDCKII-MDR1 cells were first prepared in cell seeding medium. 50 μL of cultured cell suspension was added to each well of a previously prepared Transwell plate. Incubate the plate for 4-8 days. Replace the medium every other day. The integrity of cell monolayer was assessed via electrical resistance method prior to permeability measurement.

To determine the rate of drug transport in the apical to basolateral direction. 125 μL of test compound working solution were added to the Transwell insert (apical compartment), and transferred 50 μL sample (D0 sample) immediately from the apical compartment to a new 96-well plate. To determine the rate of drug transport in the basolateral to apical direction. 285 μL of working solution of compounds are added to the receiver plate wells (basolateral compartment), and transfer 50 μL sample (DO sample) immediately from the basolateral compartment to a new 96-well plate. The plates are incubated at 37° C. for 2 hours. At the end of the transport period, transfer 50 μL directly from the apical and basolateral wells and transfer to a new plate. Then add 200 μL of cold acetonitrile containing internal standards (IS: 2 μM ketoprofen, 200 nM labetalol, 200 nM caffeine and 100 nM alprazolam) into the plate. Vortex for 5 minutes. Samples are centrifuged at 3,220 g for 20 minutes. Aliquot of 100 μL of the supernatant is diluted by 100 μL ultra-pure H2O, and the mixture is used for LC/MS/MS analysis. All incubations are performed in duplicate. The apparent permeability (Papp), in units of centimeter per second, can be calculated for MDCKII-MDR1 drug transport assays using the following equation:

$${P}_{app} = \frac{{V}_{A}}{Area \times time} \times \frac{{[ drug ]}_{acceptor}}{{[ drug ]}_{initial , donor}}$$

Where VA is the volume (in mL) in the acceptor well (0.235 mL for Ap→B1 flux and 0.075 mL for Bl→Ap flux), Area is the surface area of the membrane (0.143 cm² for Transwell-96 Well Permeable Supports), and time is the total transport time in seconds.

The efflux ratio can be determined using the following equation:

$$Efflux Ratio = \frac{{P}_{app ( B - A )}}{{P}_{app ( A - B )}}$$

Where P_{app(B-A)} indicates the apparent permeability coefficient in basolateral to apical direction, and P_{app(A-B)} indicates the apparent permeability coefficient in apical to basolateral direction.

The recovery can be determined using the following equation:

$$Recovery \% = \frac{{[ drug ]}_{acceptor} \times {V}_{A} + {[ drug ]}_{donor} \times {V}_{D}}{{[ drug ]}_{initial , donor} \times {V}_{D}} \times 100$$

Where V_{A} is the volume (in mL) in the acceptor well (0.235 mL for Ap→B1 flux, and 0.075 mL for Bl→Ap), V_{D} is the volume (in mL) in the donor well (0.075 mL for Ap→B1 flux, and 0.235 mL for Bl→Ap).

### Intrinsic Clearances in Different Species of Hepatocytes

Prepare 10 mM stock solutions of test compounds and positive control in appropriate solvent (DMSO). Place incubation medium (William's E Medium supplemented with GlutaMAX) in a 37° C. water bath, and allow warming for at least 15 minutes prior to use. In separate conical tubes, dilute the 10 mM test compound and the positive control to 100 μM by combining 198 μL of 50% acetonitrile/50% water and 2 μL of 10 mM stock. Pipette 198 μL of cryopreserved hepatocytes (0.5×10⁶ viable cells/mL) into each wells of a 96-well non-coated plate. Pipette 2 μL of the 100 μM test compounds or positive control into respective wells of the 96-well non-coated plate to start the reaction. The final concentration of test compound or control compounds is 1 μM. Return the plate to the incubator and place on an orbital shaker. Remove well contents in 25 μL aliquots at time points of 0, 15, 30, 60, 90 and 120 minutes. The aliquots are then mixed with 6 volumes (150 L) of cold acetonitrile with IS (2 μM ketoprofen, 200 nM labetalol, 200 nM caffeine and 100 nM alprazolam) to terminate the reaction. Centrifuge for 30 minutes at 3,220 g. Aliquots of 100 μL of the supernatants will be used for LC/MS/MS analysis. The supernatant may be diluted with ultrapure water according to the LC-MS signal response and peak shape. All incubations will be performed in duplicate.

All calculations are carried out using Microsoft Excel. Peak areas are determined from extracted ion chromatograms. Determine the in vitro half-life (t_{1/2}) of parent compound by regression analysis of the percent parent disappearance vs. time curve.

The in vitro half-life (in vitro t_{1/2}) is determined from the slope value:

in vitro t_{1/2}=0.693/k

Conversion of the in vitro t_{1/2} (in min) into the in vitro intrinsic clearance (in vitro CLᵢₙₜ, in μL/min/10⁶ cells) is done using the following equation:

in vitro CLᵢₙₜ=kV/N

V=incubation volume (0.2 mL); N=number of hepatocytes per well (0.1×10⁶ cells).

### Mouse and Rat PK Study

The pharmacokinetics of compounds were evaluated in male CD-1 mice or SD-JVC rats via intravenous and oral administration. For intravenous administration study, test compounds were dissolved in DMA:30% Solutol HS 15 (w/v): Saline (20:20:60, by volume) and injected with a 1 mg/kg dose via tail vein. For oral administration study, test compounds were dissolved in 0.5% MC or 0.5% MC with 0.1% Tween-80 or 30% PEG400/60% Phosal 50 PG/10% EtOH and administrated to mice at 10 mg/kg, 30 mg/kg, or 100 mg/kg by gavage. Animals will be grouped and treated according to body weight. At the time points after dosing (5 (IV only), 15, and 30 min and 1, 2, 4, 8 and 24 h after administration), Rat blood samples will be collected from JVC, Mice will be anesthetized by isoflurane and blood samples will be collected from orbital bleeding. Blood samples will be collected into 1.5 mL EDTA·K2 coated EP tube. Approximately 50 μL blood (Mouse) and 150 μL blood (Rat) were collected at each time point and placed on ice, then centrifuge at 5600 rpm 7 min at 4° C. to obtain plasma. Plasma will be transferred into new tube and stored at −20° C. or dry ice temporary. The samples will be stored at −80° C. until ex vivo PK assay.

Plasma concentrations were determined via the following sample processing method and measurement conditions. An aliquot of 10 μL sample was added with 200 μL IS (Terfenadine, 5 ng/mL) in ACN. The mixture was vortexed for 1 min, and centrifuged at 4000 rpm for 10 min at 4° C. An aliquot of 80 μL supernatant was diluted with 80 μL water, and the mixed sample was injected to liquid chromatography-tandem mass spectrometry (LC-MS/MS, Triple Quad 5500) for analysis. Injected sample amount: 2 μL. Monitor: MRM; Column: Advanced Materials Technology, HALO AQ-C18 2.7 μm 90 Å, 50*2.1 mm; Column temperature: 40° C.; Mobile phase A: H2O—0.1% FA, Mobile phase B: ACN—0.1% FA, Gradient program: 15% B-15% B (0 min-0.3 min), 15% B-90% B (0.3 min-1.0 min), 90% B-90% B (1.0 min-1.8 min), 90% B-30% B (1.8 min-2.0 min), 30% B-30% B (2.0 min-2.5 min).

### SW1990 PD Studies:

Female BALB/c Nude mice were subcutaneously implanted with 5×10⁶ SW1990 cells per 200 μL PBS/matrigel in the right flank. After inoculation, when tumors reached a mean volume of approximately 350-450 mm³ in size, mice were randomized into treatment groups. Randomized mice would receive a single dose of vehicle consisting of 0.5% MC or test compounds at various dose (e.g. 30, 50, or 100 mg/kg) by oral administration. Plasma was collected at 0.5, 2, 4, and 7 hours, and tumor was collected at 7 hours after dosing to determine exposure levels. Tumor fragments were snap frozen in homogenization tubes with liquid nitrogen and homogenized with T-PER Tissue Protein Extraction Buffer with protease and phosphatase inhibitors added fresh before use. Tumor lysates were then analyzed for ERK1/2 phosphorylation.

### SW1990 Efficacy Studies:

Female BALB/c Nude mice were subcutaneously implanted with 5×10⁶ SW1990 cells per 200 μL PBS/matrigel in the right flank. After inoculation, when tumors reached a mean volume of approximately 150-250 mm³ in size, mice were randomized into treatment groups. Randomized mice would receive vehicle consisting of 0.5% MC or test compounds at various dose (e.g. 25, 50, or 100 mg/kg BID) by oral administration. Animals were monitored daily, tumor volumes were determined twice weekly in two dimensions using a caliper, and were expressed in mm³ using the formula: V=0.5(a×b²) where a and b are the long and short diameters of the tumor, respectively. Partial regression (PR) was defined as tumor volume smaller than 50% of the starting tumor volume on the first day of dosing in three consecutive measurements and complete regression (CR) was defined as tumor volume less than 14 mm³ in three consecutive measurements. Data is presented as mean tumor volume±standard error of the mean (SEM). Tumor growth inhibition (TGI) is calculated using the following formula:

$$\% growth inhibition = 100 \times ( 1 - ( \frac{( treated t ) - ( treated to )}{( placebo t ) - ( placebo to )} ) )$$

treated t=treated tumor volume at time t
treated t₀=treated tumor volume at time 0
placebo t=placebo tumor volume at time t
placebo t₀=placebo tumor volume at time 0

### AsPC-1 Pd Studies:

Female BALB/c Nude mice were subcutaneously implanted with 3×10⁶ AsPC-1 cells per 200 μL PBS/matrigel in the right flank. After inoculation, when tumors reached a mean volume of approximately 350-450 mm³ in size, mice were randomized into treatment groups. Randomized mice would receive a single dose of vehicle consisting of 0.5% MC or test compounds at various dose (e.g. 30, 50, or 100 mg/kg) by oral administration. Plasma was collected at 0.5, 2, 4, and 8 hours, and tumor was collected at 4 and 8 hours after dosing to determine exposure levels. Tumor fragments were snap frozen in homogenization tubes with liquid nitrogen and homogenized with T-PER Tissue Protein Extraction Buffer with protease and phosphatase inhibitors added fresh before use. Tumor lysates were then analyzed for ERK1/2 phosphorylation.

### AsPC-1 Efficacy Studies:

Female BALB/c Nude mice were subcutaneously implanted with 3×10⁶ AsPC-1 cells per 200 μL PBS/matrigel in the right flank. After inoculation, when tumors reached a mean volume of approximately 150-250 mm³ in size, mice were randomized into treatment groups. Randomized mice would receive vehicle consisting of 0.5% MC or test compounds at various dose (e.g. 25, 50, or 100 mg/kg BID) by oral administration. Animals were monitored daily, tumor volumes were determined twice weekly in two dimensions using a caliper, and were expressed in mm³ using the formula: V=0.5(a×b²) where a and b are the long and short diameters of the tumor, respectively. Partial regression (PR) was defined as tumor volume smaller than 50% of the starting tumor volume on the first day of dosing in three consecutive measurements and complete regression (CR) was defined as tumor volume less than 14 mm³ in three consecutive measurements. Data is presented as mean tumor volume±standard error of the mean (SEM). Tumor growth inhibition (TGI) is calculated using the following formula:

$$\% growth inhibition = 100 \times ( 1 - ( \frac{( treated t ) - ( treated to )}{( placebo t ) - ( placebo to )} ) )$$

treated t=treated tumor volume at time t
treated t₀=treated tumor volume at time 0
placebo t=placebo tumor volume at time t
placebo t₀=placebo tumor volume at time 0

### Activity Tables

Each of the compounds in Table 2 was tested in one or more of the biochemical assays provided herein and was found to have activity therein.

TABLE 2

| | KRAS^{WT} | KRAS^{G12D} | |
|---|---|---|---|
| | Probe | Probe | AsPC-1 |
| | Displacement | Displacement | pERK |
| Example | Assay IC50 | Assay IC50 | IC₅₀ |
| No. | (nM) | (nM) | (nM) |
| Example 1 | C | B | B |
| Example 2 | C | A | A |
| Example 3 | C | A | B |
| Example 4 | C | A | A |
| Example 5 | C | A | A |
| Example 6 | C | B | B |
| Example 7 | N.A. | C | |
| Example 8 | C | B | B |
| Example 9 | N.A. | C | C |
| Example 10 | N.A. | C | |
| Example 11 | N.A. | C | |
| Example 12 | N.A. | C | |
| Example 13 | C | A | B |
| Example 14 | N.A. | C | |
| Example 15 | N.A. | B | C |
| Example 16 | C | A | A |
| Example 17 | C | A | A |
| Example 18 | C | A | A |
| Example 19 | C | A | A |
| Example 20 | C | A | B |
| Example 21 | C | B | B |
| Example 22 | C | A | B |
| Example 23 | C | A | B |
| Example 24 | C | B | C |
| Example 25 | C | B | C |
| Example 26 | C | B | C |
| Example 27 | C | B | C |
| Example 28 | C | A | B |
| Example 29 | B | A | B |
| Example 30 | C | B | B |
| Example 31 | C | B | B |
| Example 32 | C | B | C |
| Example 33 | C | A | B |
| Example 34 | C | B | B |
| Example 35 | C | B | |
| Example 36 | C | A | B |
| Example 37 | C | A | A |
| Example 38 | C | B | B |
| Example 39 | N.A. | C | |
| Example 40 | N.A. | C | C |
| Example 41 | C | B | B |
| Example 42 | C | B | B |
| Example 43 | C | B | C |
| Example 44 | B | A | A |
| Example 45 | B | A | A |
| Example 46 | C | A | A |
| Example 47 | C | A | A |
| Example 48 | C | A | B |
| Example 49 | C | A | A |
| Example 50 | C | A | A |
| Example 51 | C | A | A |
| Example 52 | B | A | A |
| Example 53 | B | A | A |
| Example 54 | B | A | A |
| Example 55 | B | A | A |
| Example 56 | C | A | A |
| Example 57 | C | B | B |
| Example 58 | C | B | B |
| Example 59 | C | A | B |
| Example 60 | C | A | B |
| Example 61 | C | A | B |
| Example 62 | C | A | A |
| Example 63 | C | A | B |
| Example 64 | B | A | A |
| Example 65 | C | A | A |
| Example 66 | C | A | A |
| Example 67 | C | A | A |
| Example 68 | C | A | B |
| Example 69 | C | A | B |
| Example 70 | B | A | B |
| Example 71 | C | A | B |
| Example 72 | C | A | B |
| Example 73 | C | A | B |
| Example 74 | C | A | B |
| Example 75 | C | A | A |
| Example 76 | C | B | C |
| Example 77 | C | A | A |
| Example 78 | C | B | C |
| Example 79 | C | A | B |
| Example 80 | C | A | C |
| Example 81 | C | A | C |
| Example 82 | C | B | |
| Example 83 | C | A | B |
| Example 84 | C | A | A |
| Example 85 | B | A | B |
| Example 86 | C | A | A |
| Example 87 | C | A | B |
| Example 88 | C | A | B |
| Example 89 | C | A | B |
| Example 90 | B | A | A |
| Example 91 | C | A | B |
| Example 92 | C | A | B |
| Example 93 | C | A | B |
| Example 94 | C | A | B |
| Example 95 | C | A | B |
| Example 96 | C | A | B |
| Example 97 | C | A | B |
| Example 98 | C | A | A |
| Example 99 | B | A | B |
| Example 100 | B | A | A |
| Example 101 | C | A | A |
| Example 102 | B | A | A |
| Example 103 | C | A | B |
| Example 104 | C | A | A |
| Example 105 | B | A | B |
| Example 106 | C | A | A |
| Example 107 | C | A | A |
| Example 108 | C | B | |
| Example 109 | N.A. | C | |
| Example 110 | C | A | A |
| Example 111 | C | A | A |
| Example 112 | C | A | A |
| Example 113 | B | A | A |
| Example 114 | B | A | A |
| Example 115 | B | A | B |
| Example 116 | C | B | B |
| Example 117 | C | A | B |
| Example 118 | C | A | B |
| Example 119 | C | A | B |
| Example 120 | B | A | A |
| Example 121 | C | A | B |
| Example 122 | C | A | A |
| Example 123 | C | A | B |
| Example 124 | C | B | C |
| Example 125 | C | A | B |
| Example 126 | C | B | ND |
| Example 127 | C | A | A |
| Example 128 | C | A | A |
| Example 129 | C | C | ND |
| Example 130 | C | B | C |
| Example 131 | C | B | C |
| Example 132 | C | B | C |
| Example 133 | C | A | B |
| Example 134 | C | A | B |
| Example 135 | C | A | B |
| Example 136 | C | A | A |
| Example 137 | C | A | A |
| Example 138 | B | A | A |
| Example 139 | C | A | A |
| Example 140 | C | A | A |
| Example 141 | C | A | A |
| Example 142 | C | A | A |
| Example 143 | C | A | B |
| Example 144 | C | A | B |
| Example 145 | C | B | C |
| Example 146 | B | A | B |
| Example 147 | C | A | B |
| Example 148 | B | A | B |
| Example 149 | C | A | B |
| Example 150 | C | A | B |
| Example 151 | C | A | B |
| Example 152 | C | A | B |
| Example 153 | C | A | C |
| Example 154 | B | A | A |
| Example 155 | C | A | B |
| Example 156 | B | A | A |
| Example 157 | C | A | A |
| Example 158 | B | A | A |
| Example 159 | B | A | A |
| Example 160 | C | A | B |
| Example 161 | C | A | B |
| Example 162 | C | A | B |
| Example 163 | C | A | B |
| Example 164 | C | A | B |
| Example 165 | B | A | A |
| Example 166 | C | A | B |
| Example 167 | B | A | B |
| Example 168 | C | A | B |
| Example 169 | C | A | B |
| Example 170 | C | A | A |
| Example 171 | C | A | B |
| Example 172 | C | A | B |
| Example 173 | C | A | B |
| Example 174 | C | A | B |
| Example 175 | C | A | B |
| Example 176 | C | A | B |
| Example 177 | B | A | A |
| Example 178 | C | A | B |
| Example 179 | C | A | B |
| Example 180 | C | B | B |
| Example 181 | C | A | B |
| Example 182 | C | A | B |
| Example 183 | C | A | B |
| Example 184 | C | A | A |
| Example 185 | C | A | B |
| Example 186 | C | A | B |
| Example 187 | C | A | B |
| Example 188 | A | A | A |
| Example 189 | C | A | A |
| Example 190 | C | A | B |
| Example 191 | C | A | B |
| Example 192 | B | A | A |
| Example 193 | C | A | B |
| Example 194 | C | A | B |
| Example 195 | B | A | A |
| Example 196 | B | A | A |
| Example 197 | C | A | B |
| Example 198 | C | A | C |
| Example 199 | B | A | A |
| Example 200 | C | A | A |
| Example 201 | B | A | A |
| Example 202 | C | A | B |
| Example 203 | C | B | B |
| Example 204 | C | A | A |
| Example 205 | C | A | B |
| Example 206 | C | A | A |
| Example 207 | B | A | A |
| Example 208 | B | A | A |
| Example 209 | C | A | A |
| Example 210 | C | A | A |
| Example 211 | C | A | A |
| Example 212 | C | A | A |
| Example 213 | C | B | B |
| Example 214 | C | A | B |
| Example 215 | C | A | ND |
| Example 216 | C | A | B |
| Example 217 | C | A | ND |
| Example 218 | B | A | A |
| Example 219 | C | A | A |
| Example 220 | C | A | A |
| Example 221 | C | A | B |
| Example 222 | C | A | B |
| Example 223 | C | A | B |
| Example 224 | C | A | B |
| Example 225 | C | A | B |
| Example 226 | C | A | B |
| Example 227 | C | A | B |
| Example 228 | C | A | B |
| Example 229 | C | A | B |
| Example 230 | C | A | B |
| Example 231 | C | A | B |
| Example 232 | B | A | B |
| Example 233 | C | B | C |
| Example 234 | C | B | B |
| Example 235 | C | B | B |
| Example 236 | C | A | ND |
| Example 237 | B | A | A |
| Example 238 | C | A | ND |
| Example 239 | B | A | A |
| Example 240 | C | B | B |
| Example 241 | B | A | A |
| Example 242 | C | A | B |
| Example 243 | C | A | A |
| Example 244 | C | B | ND |
| Example 245 | B | A | A |
| Example 246 | C | B | ND |
| Example 247 | B | A | A |
| Example 248 | B | A | A |
| Example 249 | C | A | B |
| Example 250 | C | A | B |

The activity in Table 2 is categorised as “A”, “B”, and “C” based on the corresponding value according to the following rules.

| | KRAS^{WT} | KRAS^{G12D} | |
|---|---|---|---|
| | Probe | Probe | |
| | Displace- | Displace- | |
| | ment | ment | AsPC-1 |
| | Assay | Assay | pERK |
| Categor- | IC50 | IC50 | IC₅₀ |
| ised | (nM) | (nM) | (nM) |
| A | less than | less than | less than |
| | 0.3 | 0.3 | 10 |
| B | no less | no less | no less |
| | than 0.3, | than 0.3, | than 10, |
| | and less | and less | and less |
| | than 3 | than 3 | than 100 |
| C | no less | no less | no less |
| | than 3, | than 3, | than 100, |
| | and less | and less | and less |
| | than 1000 | than 1000 | than 1000 |

As demonstrated by the data in Table 2, the inventors surprisingly and unexpectedly discovered that the exemplary compounds in Table 2 modulate or inhibit the activity of KRAS G12D.

A number of references have been cited, the disclosures of which are incorporated herein by reference in their entirety.

1. A compound having Formula (I): 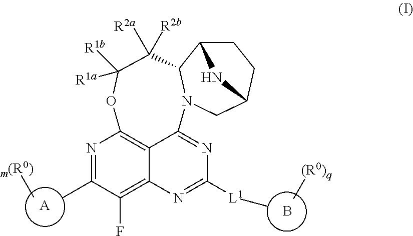
or a pharmaceutically acceptable salt, tautomer, stereoisomer, enantiomer, or atropisomer thereof,
wherein
ring A is unsubstituted or substituted aryl, or unsubstituted or substituted heteroaryl;
moiety B is unsubstituted or substituted cycloalkyl, or unsubstituted or substituted heterocyclyl;
L¹ is a direct bond, or —O—R^{a}—, wherein said R^{a} is, absent or unsubstituted or substituted C₁₋₄alkylene;
each of R⁰ is, independently, H, halogen, —CN, —OH, unsubstituted or substituted C₁₋₄alkyl, unsubstituted or substituted C₁₋₄alkoxy, unsubstituted or substituted C₁₋₄ alkenyl, unsubstituted or substituted C₃₋₅cycloalkyl, unsubstituted or substituted 3-member to 6-member heterocyclyl, or unsubstituted or substituted amino; or one or more pairs of the R⁰ groups, together with the atom(s) to which they are attached to, form unsubstituted or substituted cycloalkyl, unsubstituted or substituted alkenyl, unsubstituted or substituted heterocyclyl, unsubstituted or substituted aryl, or unsubstituted or substituted heteroaryl;
each of R^{1a}, R^{1b}, R^{2a} and R^{2b}, is, independently, H, halogen, unsubstituted or substituted C₁₋₃alkyl, or R^{1a} and R^{1b}, together form an oxo or a substituted or unsubstituted cyclopropyl;
each of m, and q is, independently, an integer from 0 to the maximum number of the substituent groups allowed on rings A, and B, respectively;
provided said compound is not the compound of Table 1.

2. The compound of claim 1, wherein the compound of formula (I) is a compound of formula (IIa):

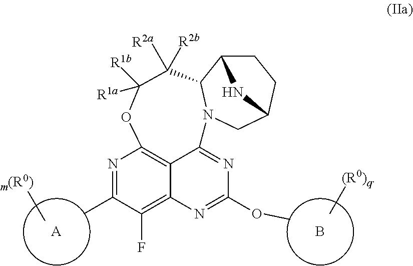

3. The compound of claim 1, wherein the compound of formula (I) is a compound of formula (IIb): 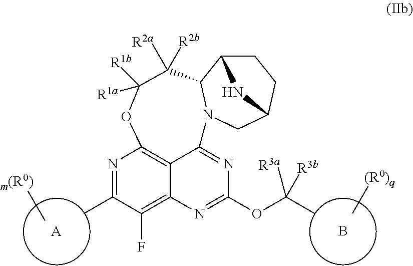
wherein
each of R^{3a} and R^{3b}, is, independently, H, F, unsubstituted or substituted C₁₋₂alkyl.

4. The compound of claim 3, wherein moiety B is 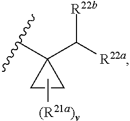
wherein
each of R^{21a} is, independently, H or halogen;
each of R^{22a} and R^{22b} is, independently, H, substituted or unsubstituted alkyl, substituted or unsubstituted amino or substituted or unsubstituted heterocyclyl, or R^{22a} and R^{22b}, together with the atom which R^{22a} and R^{22b} connect with, form a substituted or unsubstituted heterocyclyl; and
v is 0, 1, 2, 3, or 4.

5. The compound of claim 4, wherein moiety B is 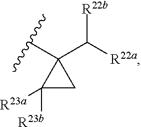
wherein
each of R^{23a} and R^{23b} is, independently, H or halogen;
each of R^{22a} and R^{22b} is, independently, H, substituted or unsubstituted alkyl, substituted or unsubstituted amino or substituted or unsubstituted heterocyclyl, or R^{22a} and R^{22b}, together with the atom which R^{22a} and R^{22b} connect with, form a substituted or unsubstituted heterocyclyl.

6. The compound of claim 5, wherein moiety B is 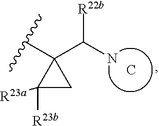
wherein ring C is substituted or unsubstituted 4- to 10-membered heterocyclyl optionally containing one or more additional heteroatom selected from N, O, or S.

7. The compound of claim 3, wherein moiety B is 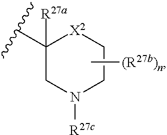
wherein
X² is —CHF—, —CF₂—, —CH(CH₃)—, N(CH₃)—, —CH(OCH₃)—, —CH(CHF₂)—, —C(═CHF)—, or —O—;
R^{27a} is methyl, ethyl, difluoromethyl, or trifluoromethyl;
R^{27b} is H, F, methyl, or methoxy;
R^{27c} is C₁₋₃alkyl, oxetanyl, tetrahydrofuranyl, or tetrahydropyranyl, wherein said C₁₋₃alkyl is optionally substituted with one or more substituents selected from methyl, methyl-d3, F or alkenyl; and
n is an integer from 0 to 4.

8. The compound of any one of claim 3, wherein moiety B is 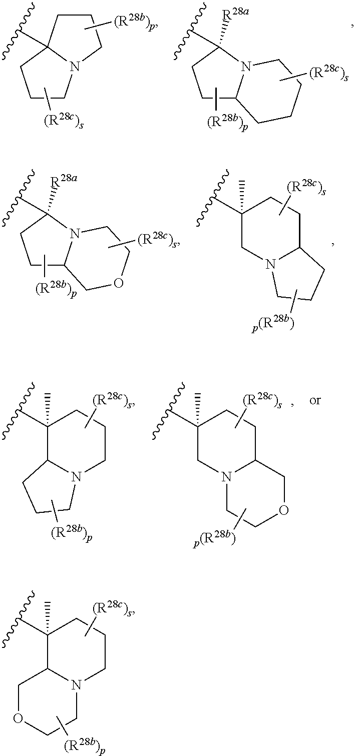
wherein
R^{28a} is methyl, methyl-d3, fluoromethyl, difluoromethyl, or trifluoromethyl;
each of R^{28b} and R^{28c} is H, halogen, substituted or unsubstituted alkyl, or one pair of R^{28b} or R^{28c} together with the atom(s) to which they are attached to, form substituted or unsubstituted alkenylene, or unsubstituted or substituted cycloalkyl; and
each of s and p is, independently, 0, 1, 2, 3, or 4;
provided 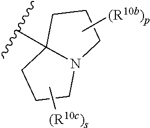
is not

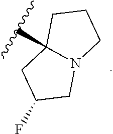 [SMILES: *[C@@]12CCCN1C[C@H](F)C2]

9. The compound of claim 3, wherein moiety B is 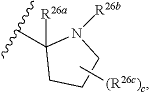
wherein
R^{26a} is H, methyl, or difluoromethyl;
R^{26b} is methyl, methyl-d3, ethyl, 2-fluoroethyl, 2-(methoxy-d3)ethyl, 1-allyl-2-methyl, 2-methoxyethyl, 1-allyl-2-methyl, cyclopropylmethyl, oxetanyl, tetrahydrofuranyl, or tetrahydro-2H-pyranyl;
each R^{26c} is, independently, H, F or methoxy, or one pair of R^{26c}, together with the atom to which they are attached form a substituted or unsubstituted cyclopropyl; and
c is 0, 1, or 2.

10. The compound of claim 6, wherein the compound is a compound of formula (IVb): 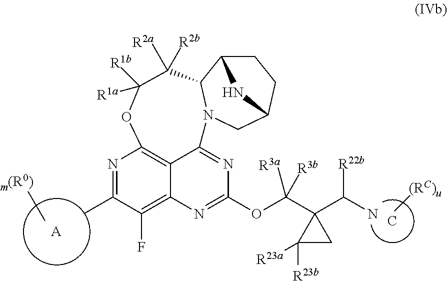
wherein
ring C is is substituted or unsubstituted 4- to 7-membered heterocyclyl optionally containing one or more additional heteroatom selected from N or O;
each of R^{c} is, independently, H, halogen, amino, —OH, —CN, unsubstituted or substituted C₁₋₄alkyl, or unsubstituted or substituted C₁₋₄alkoxy, or one pair of the R^{c} groups, together with the atom(s) to which they are attached to, form unsubstituted or substituted bridge, unsubstituted or substituted cycloalkyl, or unsubstituted or substituted heterocyclyl.
u is an integer from 0 to the maximum number of the substituent groups allowed on rings C.

11. The compound of claim 8, wherein the compound is a compound of formula (IVb): 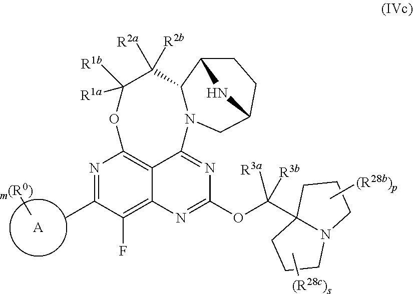
wherein
each of R^{28b} and R^{28c} is H, halogen, substituted or unsubstituted alkyl, substituted or unsubstituted alkoxy, or one pair of R^{28b} or R^{28c} together with the atom(s) to which they are attached to, form unsubstituted or substituted alkenyl, or unsubstituted or substituted cycloalkyl; and
each of s and p is, independently, 0, 1, 2, 3, or 4; and
provided 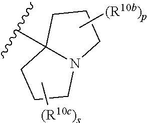
is not

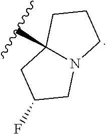 [SMILES: *[C@@]12CCCN1C[C@H](F)C2]

12. The compound of claim 1, wherein the compound of formula (I) is a compound of formula (IIc): 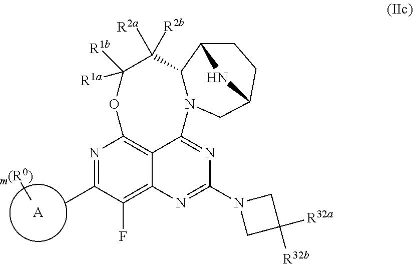
wherein
each of R^{32a} and R^{32b}, is, independently, H, unsubstituted or substituted C₁₋₄alkyl, unsubstituted or substituted amino, or unsubstituted or substituted heterocyclyl.

13. The compound, wherein the compound is one of the compounds in Table 2.

14. A pharmaceutical composition comprising a compound of claim 1, or a pharmaceutically acceptable salt, tautomer, stereoisomer, enantiomer, or atropisomer thereof, and a pharmaceutically acceptable carrier, excipient or vehicle.

15. A method for inhibiting the activity of KRAS mutant protein in a cell, comprising contacting said cell with a compound of claim 1, or a pharmaceutically acceptable salt, tautomer, stereoisomer, enantiomer, or atropisomer thereof, optionally wherein the KRAS mutant protein is KRAS G12D mutant protein.

16. A method for treatment or prevention of cancer, the method comprising administering to a subject in need thereof a compound of claim 1, or a pharmaceutically acceptable salt, tautomer, stereoisomer, enantiomer, or atropisomer thereof, optionally wherein the cancer is mediated by KRAS mutation; preferably KRAS G12D mutation.
